(19) United States
(12) Patent Application Publication
(10) Pub. No.: US 2026/0042875 A1
(43) Pub. Date: Feb. 12, 2026

(54) SULFONIUM SALT MONOMER, POLYMER, CHEMICALLY AMPLIFIED RESIST COMPOSITION, AND PATTERN FORMING PROCESS

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 19/285,660

(22) Filed: Jul. 30, 2025

(30) Foreign Application Priority Data
Aug. 8, 2024 (JP) 2024-131999

(51) Int. Cl. C08F 220/22; C08F 212/14; C08F 212/32; C08F 220/18; C08F 220/26; G03F 7/004; G03F 7/039

(52) U.S. Cl. CPC C08F 220/22; C08F 212/20; C08F 212/24; C08F 212/32; C08F 220/1806; C08F 220/26; G03F 7/0045; G03F 7/0392

(57) ABSTRACT

A sulfonium salt monomer having the formula (A). The sulfonium salt monomer can be used for a chemically amplified resist composition having a high solvent solubility, a high sensitivity, and a high contrast, and being improved in lithography properties such as EL, LWR, CDU, and DOF, particularly when processed by photolithography using high-energy radiation such as KrF excimer laser, ArF excimer laser, an electron beam (EB), or EUV.

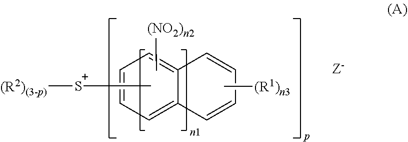

### CROSS-REFERENCE TO RELATED APPLICATION

This non-provisional application claims priority under 35 U.S.C. § 119 (a) on Patent Application No. 2024-131999 filed in Japan on Aug. 8, 2024, the entire contents of which are hereby incorporated by reference.

### TECHNICAL FIELD

The invention relates to a sulfonium salt monomer, a polymer, a chemically amplified resist composition, and a pattern forming process.

### BACKGROUND ART

To meet the demand for higher integration density and higher operating speed of LSIs, the effort to reduce the pattern rule is in rapid progress. In particular, the wide-spreading flash memory market and the demand for increased storage capacities drive forward the miniaturization technology. As the advanced miniaturization technology, manufacturing of microelectronic devices at the 65-nm node by the ArF lithography has been implemented in a mass scale. Manufacturing of 45-nm node devices by the next generation ArF immersion lithography is approaching to the verge of high-volume application. The candidates for the next generation 32-nm node include ultra-high NA lens immersion lithography using a liquid having a higher refractive index than water in combination with a high refractive index lens and a high refractive index resist film, extreme ultraviolet (EUV) lithography of 13.5 nm wavelength, and double exposure (double patterning lithography) of the ArF lithography, on which active research efforts have been made.

As the pattern feature size is reduced, approaching to the diffraction limit of light, light contrast lowers. In the case of positive resist film, a lowering of light contrast leads to reductions of resolution and focus margin of hole and trench patterns.

As the pattern feature size is reduced, the line width roughness (LWR) of line patterns and the critical dimension uniformity (CDU) of hole patterns are regarded significant. It is pointed out that these factors are affected by the segregation or agglomeration of a base polymer and acid generator and the diffusion of generated acid. Furthermore, there is a tendency that as the resist film becomes thinner, LWR becomes greater. A film thickness reduction to comply with the progress of size reduction causes a degradation of LWR, which becomes a serious problem.

The EUV lithography resist compositions must meet high sensitivity, high resolution and low LWR at the same time. As the acid diffusion distance is reduced, LWR is reduced, but sensitivity becomes lower. For example, as the post exposure bake (PEB) temperature is lowered, the outcome is a reduced LWR, but a lower sensitivity. As the amount of quencher added is increased, the outcome is a reduced LWR, but a lower sensitivity. It is necessary to overcome the tradeoff relation between sensitivity and LWR.

Patent Document 1 discloses a resist compound comprising repeat units derived from an onium salt of a polymerizable unsaturated bond-containing sulfonic acid in order to suppress acid diffusion. The so-called polymer-bound acid generator is capable of generating a polymeric sulfonic acid upon exposure and characterized by a very short distance of acid diffusion. Sensitivity may be enhanced by increasing a proportion of the acid generator. In the case of addition type acid generators, as the amount of acid generator added is increased, a higher sensitivity is achievable, but the acid diffusion distance is also increased. Since the acid diffusion is non-uniform, increased acid diffusion leads to degraded LWR and CDU. With respect to a balance of sensitivity, LWR and CDU, the polymeric acid generator has a high capability.

It is known that iodine atoms are highly absorptive to EUV of wavelength 13.5 nm, and thus generate secondary electrons upon light exposure. This phenomenon is attractive in the EUV lithography. Patent Document 2 describes a photoacid generator containing an anion having iodine introduced therein, and Patent Document 3 describes a polymerizable group-containing photoacid generator containing an anion having iodine introduced therein. As a result, although the lithography properties are improved to some extent, iodine atoms are not so high in organic solvent solubility, and there is concern for precipitation in the solvent.

Patent Documents 4 and 5 describe photoacid generators in which a pentafluorosulfanyl group (—SF₅ group) or a trifluoromethoxy group (—OCF₃ group) is introduced into a cation. As a result, although lithography properties has been improved to some extent, there is still room for improvement, and development of a resist material effective for finer pattern formation is desired.

### CITATION LIST

Patent Document 1: JP 4425776
Patent Document 2: JP 6720926
Patent Document 3: JP 6973274
Patent Document 4: WO 2023/223624
Patent Document 5: JP-A 2022-059112

### SUMMARY OF THE INVENTION

In a chemically amplified resist composition using an acid as a catalyst, it is desired to develop a resist composition having a higher sensitivity and improved lithography properties such as exposure tolerance (EL), LWR, CDU, and depth of focus (DOF).

An object of the invention is to provide a sulfonium salt monomer which is used for a chemically amplified resist composition having a high solvent solubility, a high sensitivity, and a high contrast, and being improved in lithography properties such as EL, LWR, CDU, and DOF, particularly when processed by photolithography using high-energy radiation such as KrF excimer laser, ArF excimer laser, an electron beam (EB), or EUV, a polymer comprising repeat units derived from the sulfonium salt monomer, a chemically amplified resist composition comprising the polymer, and a pattern forming process using the chemically amplified resist composition.

The inventors have found that by using a polymer comprising repeat units derived from a sulfonium salt monomer of a sulfonium cation having a nitro group and a fluorosulfonate anion having an aromatic vinyl structure and an iodine atom as polymer-bonded acid generators, a chemically amplified resist composition having a high sensitivity, improved lithography properties such as EL, LWR, CDU, and DOF, a high contrast, and high resolution is obtained.

That is, the invention provides the following sulfonium salt monomer, polymer, chemically amplified resist composition, and pattern forming process.
1. A sulfonium salt monomer having the formula (A):

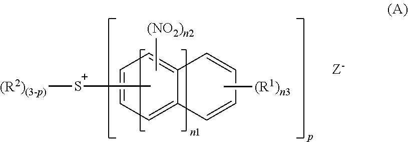

wherein p is 1, 2, or 3, n1 is 0 or 1, n2 is 1 or 2, n3 is 0, 1, 2, or 3, meeting 1≤n2+n3≤5 in case of n1=0 and 1≤n2+n3≤7 in case of n1=1,
   R¹ is halogen, cyano, hydroxy, carboxy, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom, and when n3 is 2 or 3, a plurality of R¹ may be identical or different and two R¹ may bond together to form a ring with the carbon atoms to which they are attached,
   R² is halogen or a C₁-C₃₀ hydrocarbyl group which may contain a heteroatom, when p is 1, two R² may be identical or different, and any two of three substituents bonded to S⁺ may bond together to form a ring with the sulfur atoms to which they are attached, and
   Z⁻ is a fluoroalkanesulfonate anion having an aromatic vinyl structure and an iodine atom.
2. The sulfonium salt monomer of 1 having the formula (A1):

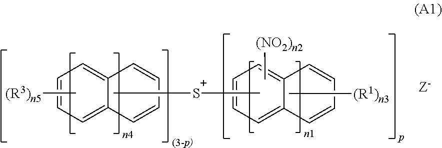

wherein p, n1 to n3, R¹, and Z⁻ are as defined above,
   n4 is 0 or 1, n5 is 0, 1, 2, 3, 4, or 5, and
   R³ is halogen, cyano, hydroxy, carboxy, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom, and when n5 is 2, 3, 4, or 5, a plurality of R³ may be identical or different and two R³ may bond together to form a ring with the carbon atoms to which they are attached.
3. The sulfonium salt monomer of 1 or 2 wherein Z⁻ is an anion having the formula (Z):

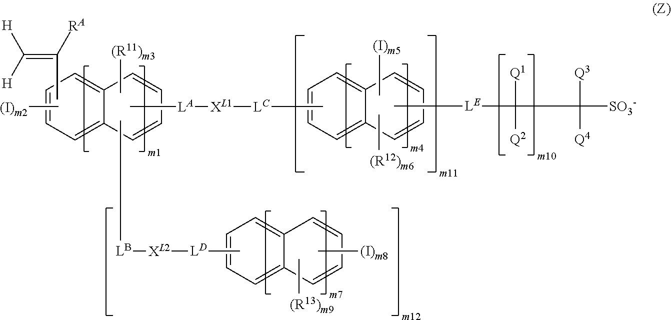

wherein m1 is 0 or 1, m2 is 0, 1, 2, 3, or 4, m3 is 0, 1, 2, or 3, m4 is 0 or 1, m5 is 0, 1, 2, 3, or 4, m6 is 0, 1, 2, or 3, m7 is 0 or 1, m8 is 1, 2, 3, or 4, m9 is 0, 1, 2, or 3, m10 is 0, 1, 2, 3, or 4, m11 is 0 or 1, m12 is 0 or 1, meeting 0≤m2+m3+m12≤4 in case of m1=0 and 0≤m2+m3+m12≤6 in case of m1=1, 0≤m5+m6≤4 in case of m4-0 and 0≤m5+m6≤6 in case of m4=1, 0≤m8+m9≤5 in case of m7=0 and 0≤m8+m9≤7 in case of m7=1, and also meeting 1≤m2+m5+m8≤4,
   R^{A} is hydrogen, fluorine, methyl or trifluoromethyl,
   R¹¹, R¹², and R¹³ are each independently halogen exclusive of iodine, nitro, cyano, hydroxy, carboxy, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom, when m3 is 2 or 3, a plurality of R¹¹ may be identical or different and two R¹¹ may bond together to form a ring with the carbon atoms to which they are attached, when m6 is 2 or 3, a plurality of R¹² may be identical or different and two R¹² may bond together to form a ring with the carbon atoms to which they are attached, and when m9 is 2 or 3, a plurality of R¹³ may be identical or different and two R¹³ may bond together to form a ring with the carbon atoms to which they are attached,
   L^{A}, L^{B}, L^{C}, L^{D}, and L^{E} are each independently a single bond, ether bond, ester bond, sulfonate ester bond, amide bond, sulfonamide bond, carbonate bond or carbamate bond,
   X^{L1} and X^{L2} are each independently a single bond or a C₁-C₄₀ hydrocarbylene group which may contain a heteroatom,
   Q¹ and Q² are each independently hydrogen, fluorine, or a C₁-C₆ fluorinated saturated hydrocarbyl group, and
   Q³ and Q⁴ are each independently fluorine or a C₁-C₆ fluorinated saturated hydrocarbyl group,
   excluding that m11 and m12 are 0 at the same time, and excluding that L^{A}, L^{B}, L^{C}, L^{D}, X^{L1} and X^{L2} are a single bond at the same time.
4. A monomer photoacid generator comprising the sulfonium salt monomer of any one of 1 to 3.
5. A polymer comprising repeat units derived from the monomer photoacid generator of 4.
6. The polymer of 5, further comprising at least one selected from repeat units having the formula (a1), repeat units represented by the formula (a2), and repeat units represented by the formula (a3):

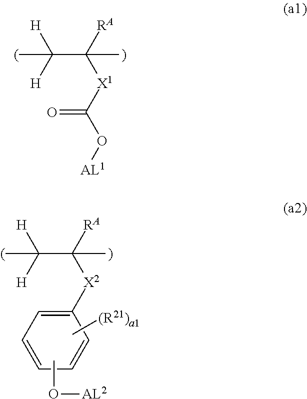

wherein R^{A} is each independently hydrogen, fluorine, methyl or trifluoromethyl,
   X¹ is a single bond, phenylene group, naphthylene group, or *—C(═O)—O—X¹¹—, the phenylene or naphthylene group may be substituted by a hydroxy moiety, nitro moiety, cyano moiety, optionally fluorinated C₁-C₁₀ saturated hydrocarbyl moiety, optionally fluorinated C₁-C₁₀ saturated hydrocarbyloxy moiety, or halogen, X¹¹ is a C₁-C₁₀ saturated hydrocarbylene group which may contain a hydroxy moiety, ether bond, ester bond or lactone ring, or phenylene or naphthylene group,
   X² is a single bond or *—C(═O)—O—,
   the asterisk (*) designates a point of attachment to the carbon atom in the backbone,
   R²¹ is halogen, cyano, hydroxy, nitro, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R²¹ may be identical or different when a1 is 2, 3, or 4,
AL¹ and AL² are each independently an acid labile group, and
   a1 is 0, 1, 2, 3, or 4;

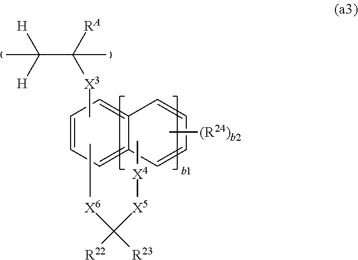

wherein b1 is 0 or 1, b2 is 0, 1, 2, or 3 in case of b1=0, and is 0, 1, 2, 3, 4, or 5 in case of b1=1,
R^{A} is hydrogen, fluorine, methyl or trifluoromethyl,
X³ is a single bond, *—C(═O)—O—, or *—C(═O)—NH—, the asterisk (*) designates a point of attachment to the carbon atom in the backbone,
X⁴ is a single bond, a C₁-C₄ aliphatic hydrocarbylene group, carbonyl group, sulfonyl group, or a group obtained by combining the foregoing,
X⁵ and X⁶ are each independently oxygen or sulfur, X⁴ and X⁶ are bonded to vicinal carbon atoms on the aromatic ring,
R²² and R²³ are each independently hydrogen or a C₁-C₂₀ hydrocarbyl group which may contain a heteroatom, R²² and R²³ may bond together to form a ring with the carbon atoms to which they are attached, and
R²⁴ is halogen, hydroxy, cyano, nitro, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{24A})(R^{24B}), R^{24A} and R^{24B} are each independently hydrogen or a C₁-C₆ hydrocarbyl group, and when b2 is 2 or more, a plurality of R²⁴ may be identical or different and a plurality of R²⁴ may bond together to form a ring with the carbon atoms on the aromatic ring to which they are attached.
7. The polymer of 5 or 6, further comprising at least one selected from repeat units having the formula (b1) and repeat units having the formula (b2):

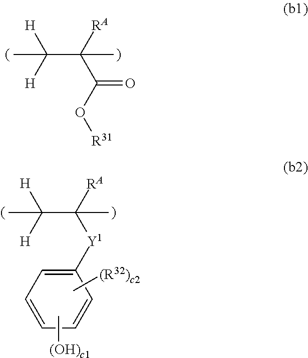

wherein R^{A} is each independently hydrogen, fluorine, methyl or trifluoromethyl,
   Y¹ is a single bond or *—C(═O)—O—, the asterisk (*) designates a point of attachment to the carbon atom in the backbone,
   R³¹ is hydrogen or a C₁-C₂₀ group containing at least one structure selected from among hydroxy other than phenolic hydroxy, cyano, carbonyl, carboxy, ether bond, ester bond, sulfonate ester bond, carbonate bond, lactone ring, sultone ring and carboxylic anhydride (—C(═O)—O—C(═O)—),
   R³² is halogen, carboxy, nitro, cyano, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R³² may be identical or different when c2 is 2, 3, or 4, and
   c1 is 1, 2, 3, or 4 and c2 is 0, 1, 2, 3, or 4, meeting 1≤c1+c2≤5.
8. A chemically amplified resist composition comprising (A) a base polymer comprising the polymer of any one of 5 to 7.
9. The chemically amplified resist composition of 8, further comprising (B) an organic solvent.
10. The chemically amplified resist composition of 8 or 9, further comprising (C) a quencher.
11. The chemically amplified resist composition of any one of 8 to 10, further comprising (D) a photoacid generator.
12. The chemically amplified resist composition of any one of 8 to 11, further comprising (E) a surfactant.
13. A pattern forming process comprising the steps of applying the chemically amplified resist composition of any one of 8 to 12 onto a substrate to form a resist film thereon, exposing the resist film to high-energy radiation, and developing the exposed resist film in a developer.
14. The pattern forming process of 13 wherein the high-energy radiation is KrF excimer laser, ArF excimer laser, EB, or EUV of wavelength 3 to 15 nm.

### Advantageous Effects of the Invention

When pattern is formed using a chemically amplified resist composition comprising a polymer comprising repeat units functioning as a photoacid generator derived from the sulfonium salt monomer of the invention, it is possible to form a resist pattern having a high contrast, a good sensitivity, and improved lithography properties such as EL, LWR, CDU, and DOF.

### DESCRIPTION OF THE PREFERRED EMBODIMENT

Hereinafter, the invention is described in detail. In the following description, an asymmetric carbon may exist and an enantiomer or a diastereomer may exist depending on the structure represented by the chemical formula. In that case, these isomers are represented by one formula as a representative. These isomers may be used alone or in admixture of two or more.

### Sulfonium Salt Monomer

The sulfonium salt monomer of the invention has the formula (A).

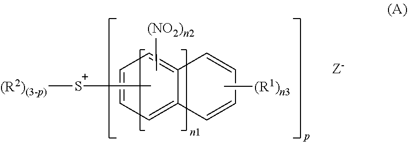

In the formula (A), p is 1, 2, or 3.

In the formula (A), n1 is 0 or 1. The relevant structure is a benzene ring in case of n1=0, and a naphthalene ring in case of n1=1. The benzene ring corresponding to n1=0 is preferred from the aspect of solvent solubility. n2 is 1 or 2. n2 is preferably 1 from the aspect of availability of reactants. n3 is 0, 1, 2, or 3. n3 is preferably 0, 1 or 2 from the aspect of availability of reactants. These subscripts meet 1≤n2+n3≤5 in case of n1=0 and 1≤n2+n3≤7 in case of n1=1.

In the formula (A), R¹ is halogen, cyano, hydroxy, carboxy, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom. Examples of the halogen include fluorine, chlorine, bromine, and iodine. The hydrocarbyl group and hydrocarbyl moiety in the hydrocarbyloxy and hydrocarbylthio groups may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, 1-propenyl, 2-propenyl, butenyl, and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl, and 2-phenylethyl; and combinations thereof. Of these, aryl groups are preferred. In the hydrocarbyl group, some or all of the hydrogen atoms may be substituted by a moiety containing a heteroatom such as oxygen, sulfur, nitrogen or halogen, and some constituent —CH²— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety. A plurality of R¹ may be identical or different when n3 is 2 or 3. Also, when n3 is 2 or 3, a plurality of R¹ may bond together to form a ring with the carbon atoms to which they are attached. The ring is preferably a 5- to 8-membered ring.

In the formula (A), R² is halogen or a C₁-C₃₀ hydrocarbyl group which may contain a heteroatom. When p is 1, two R² may be identical or different.

Examples of the halogen represented by R² include fluorine, chlorine, bromine, and iodine.

The hydrocarbyl group represented by R² may be saturated or unsaturated and straight, branched or cyclic. Examples thereof include C₁-C₃₀ alkyl groups such as methyl, ethyl, n-propyl, isopropyl, n-butyl, isobutyl, sec-butyl, and tert-butyl; C₃-C₃₀ cyclic saturated hydrocarbyl groups such as cyclopropyl, cyclopentyl, cyclohexyl, cylopropylmethyl, 4-methylcyclohexyl, cyclohexylmethyl, norbornyl, and adamantyl; C₂-C₃₀ alkenyl groups such as vinyl, 1-propenyl, 2-propenyl, butenyl, and hexenyl; C₃-C₃₀ cyclic unsaturated hydrocarbyl groups such as cyclohexenyl; C₆-C₃₀ aryl groups such as phenyl, naphthyl, and thienyl; C₇-C₃₀ aralkyl groups such as benzyl, 1-phenylethyl, and 2-phenylethyl; and combinations thereof. Of these, aryl groups are preferred. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, nitro moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

Any two of three substituents bonded to S⁺ may bond together to form a ring with the sulfur atoms to which they are attached. Examples of the structure of the ring include structures having the formulae:

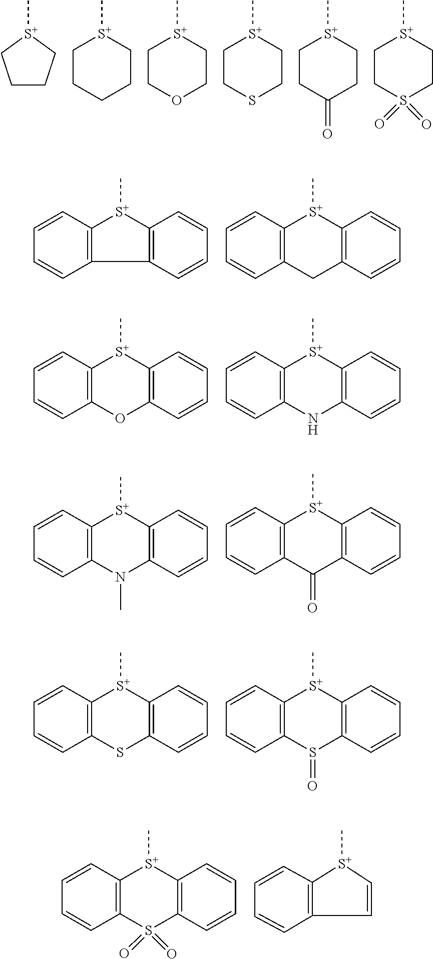

wherein the broken line is a valence bond.

As the sulfonium salt monomer having the formula (A), a sulfonium salt monomer having the formula (A1) is preferred.

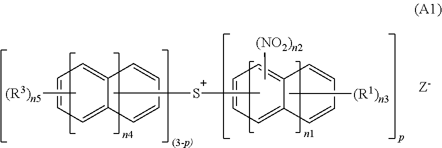

Herein p, n1 to n3, and R¹ are as defined above, and Z-will be described later.

In the formula (A1), n4 is 0 or 1. The relevant structure is a benzene ring in case of n4=0, and a naphthalene ring in case of n4=1. The benzene ring corresponding to n4-0 is preferred from the aspect of solvent solubility. n5 is 0, 1, 2, 3, 4, or 5. n5 is preferably 0, 1 or 2 from the aspect of availability of reactants.

In the formula (A1), R³ is halogen, cyano, hydroxy, carboxy, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom. The hydrocarbyl group and hydrocarbyl moiety in the hydrocarbyloxy and hydrocarbylthio groups may be saturated or unsaturated and straight, branched or cyclic. Examples thereof include those exemplified above for the hydrocarbyl group represented by R¹, but not limited thereto. When n5 is 2, 3, 4, or 5, a plurality of R³ may be identical or different and two R³ may bond together to form a ring with the carbon atoms on the aromatic ring to which they are attached.

Examples of the sulfonium salt monomer cation having the formula (A) include those shown below, but not limited thereto.

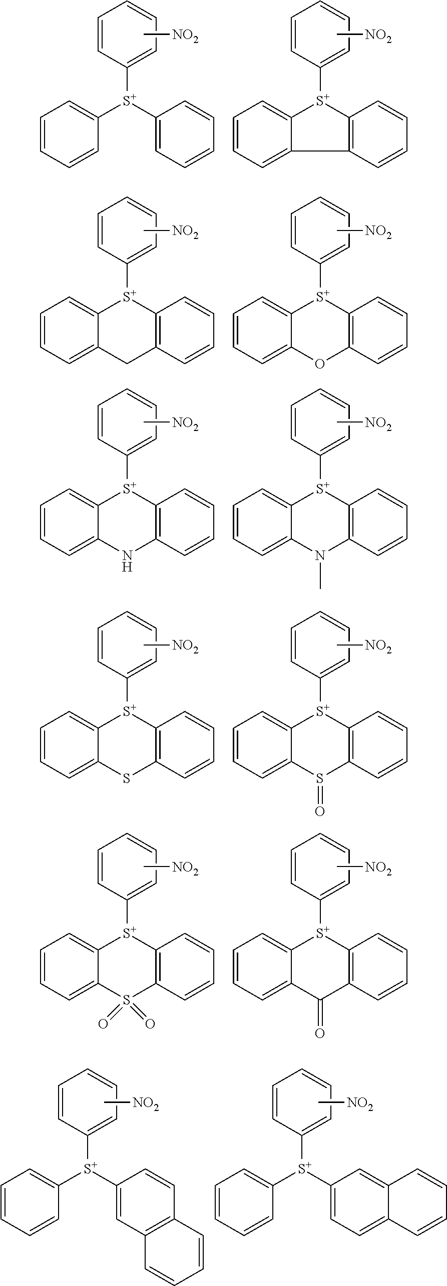

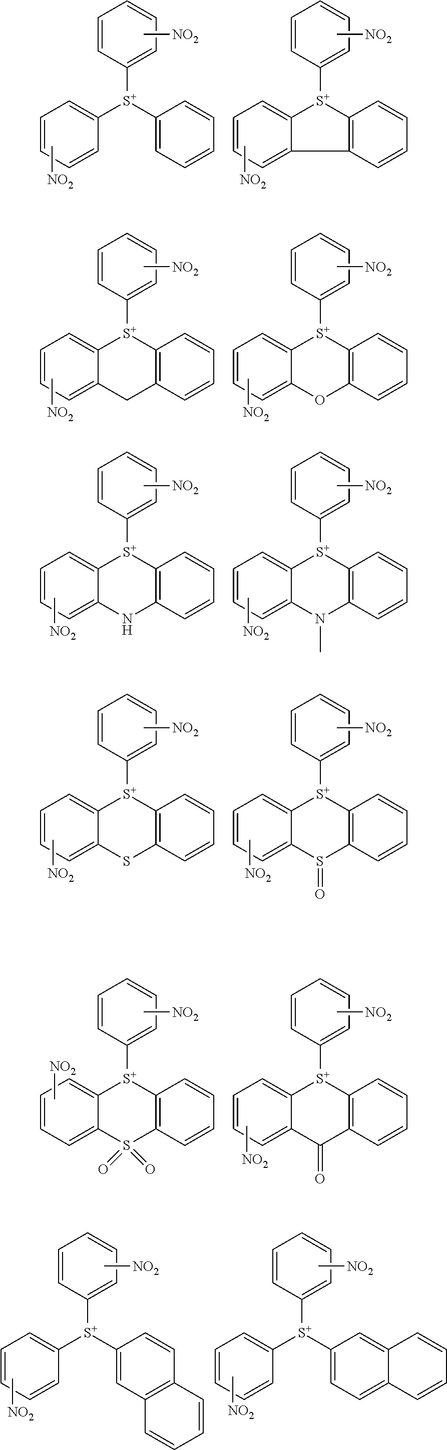

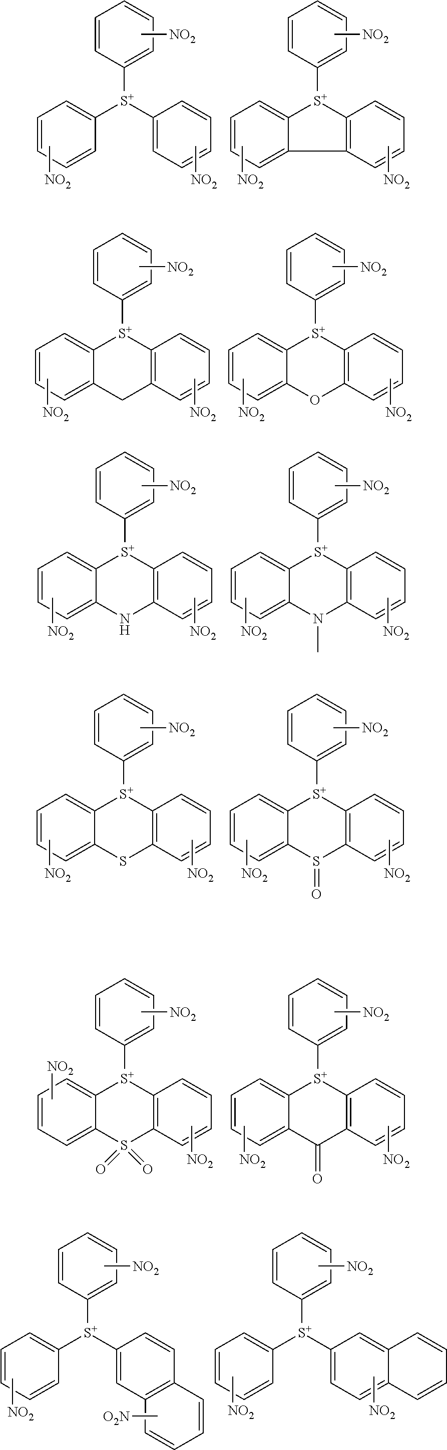

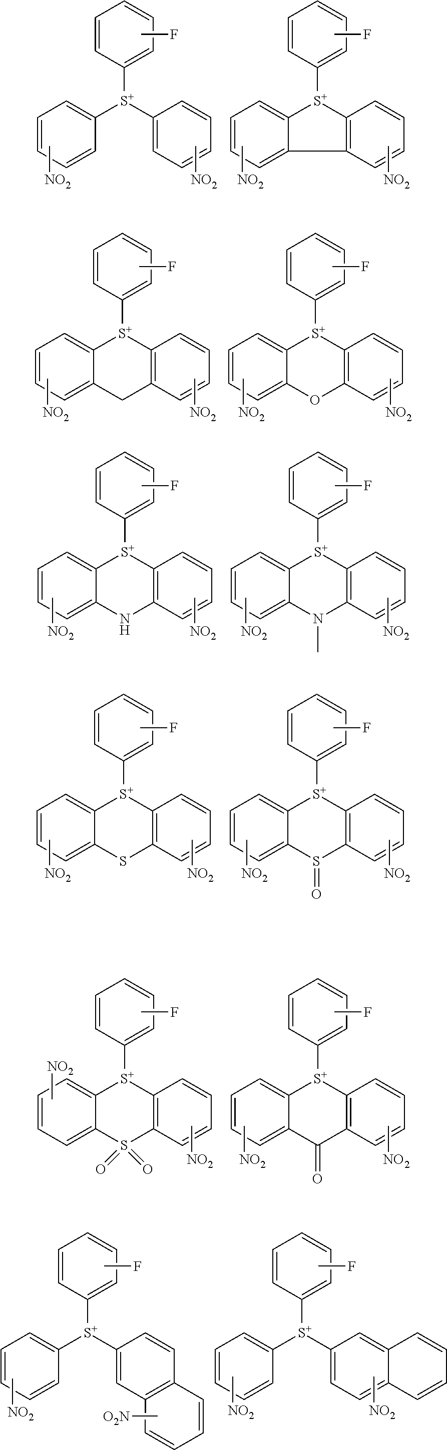

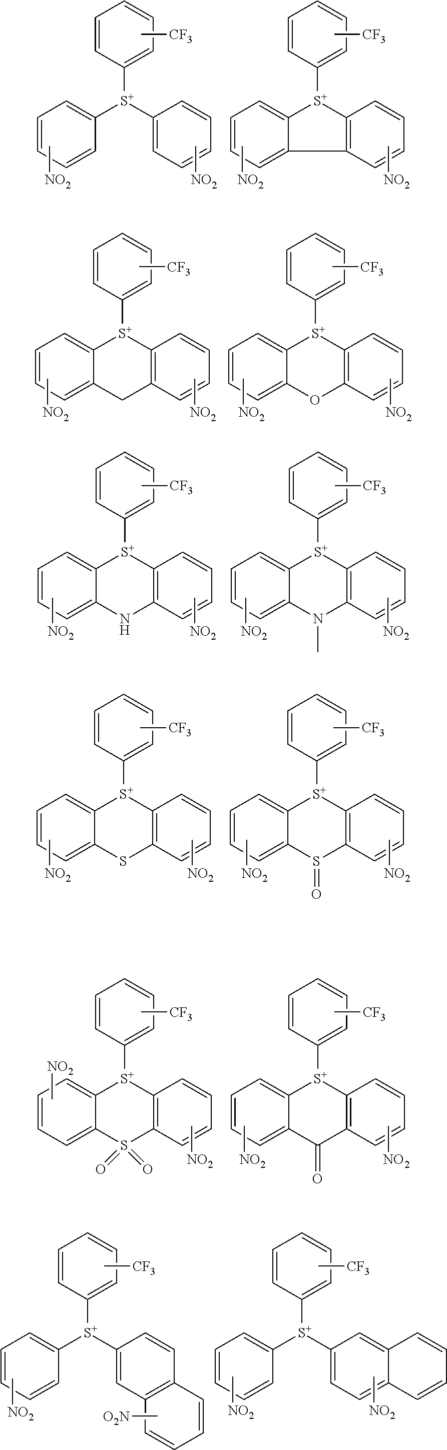

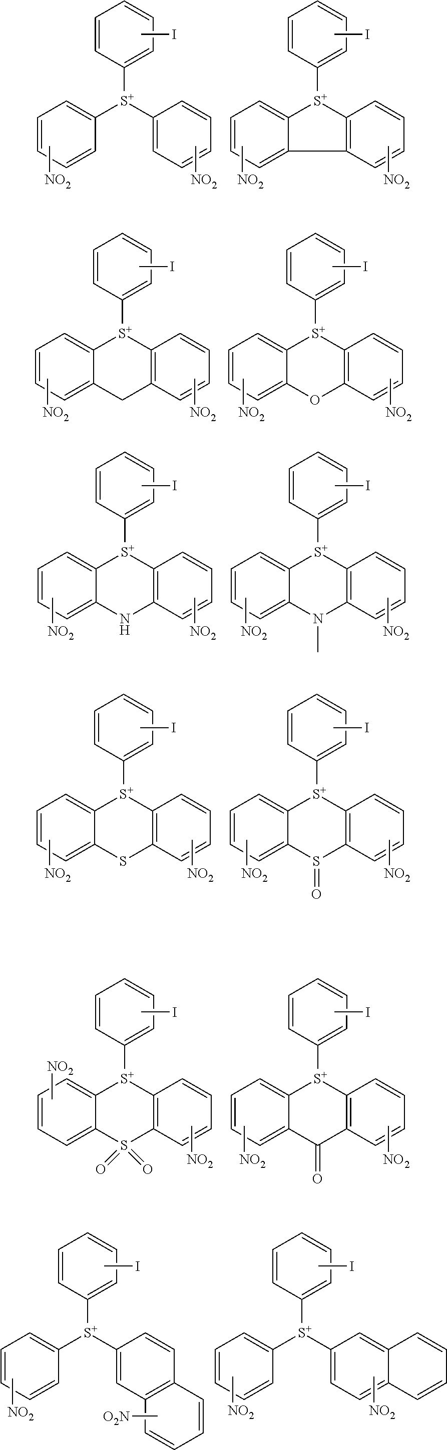

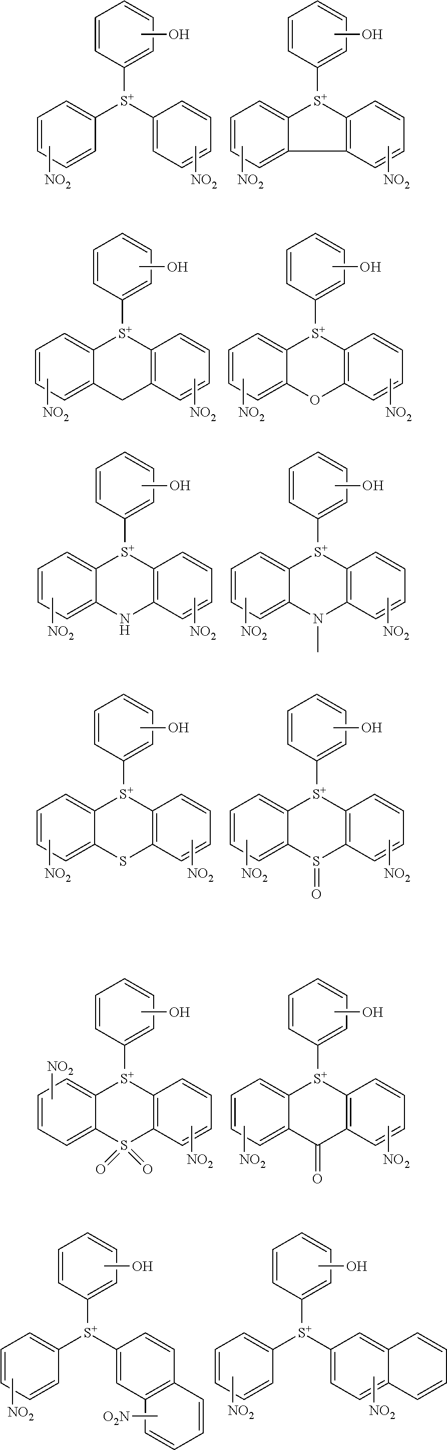

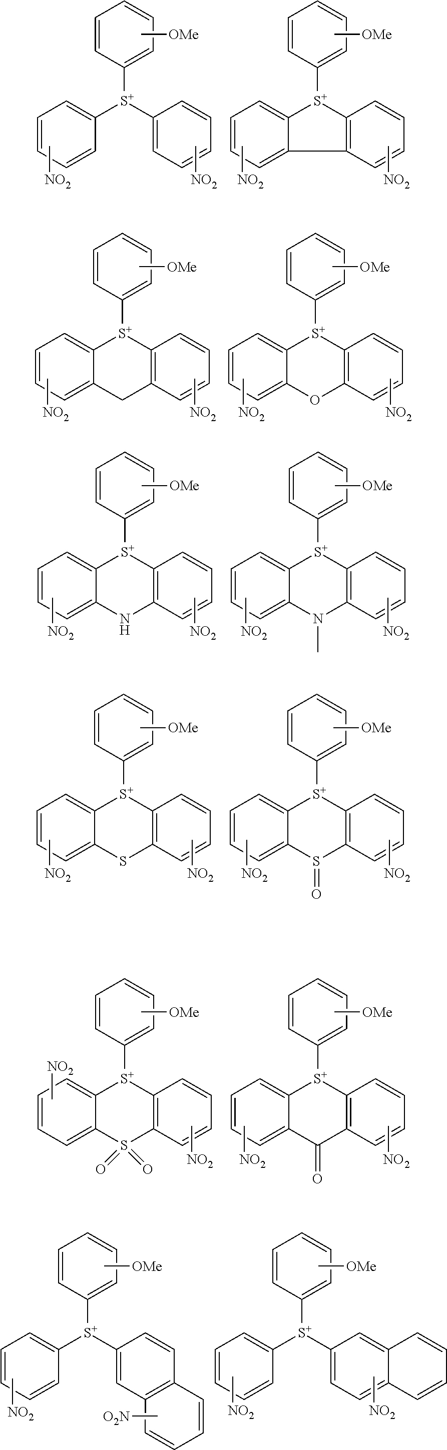

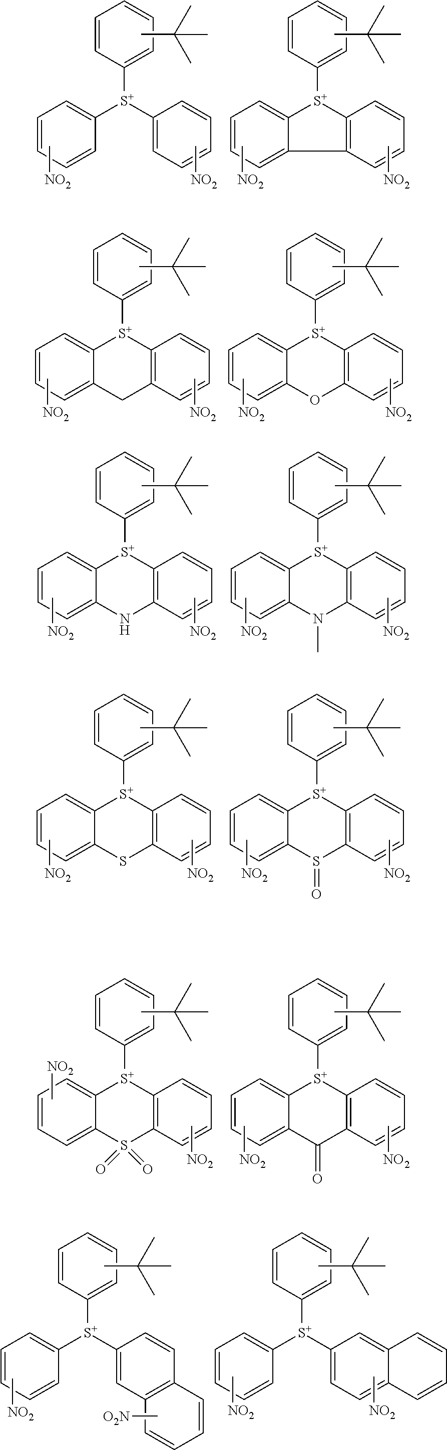

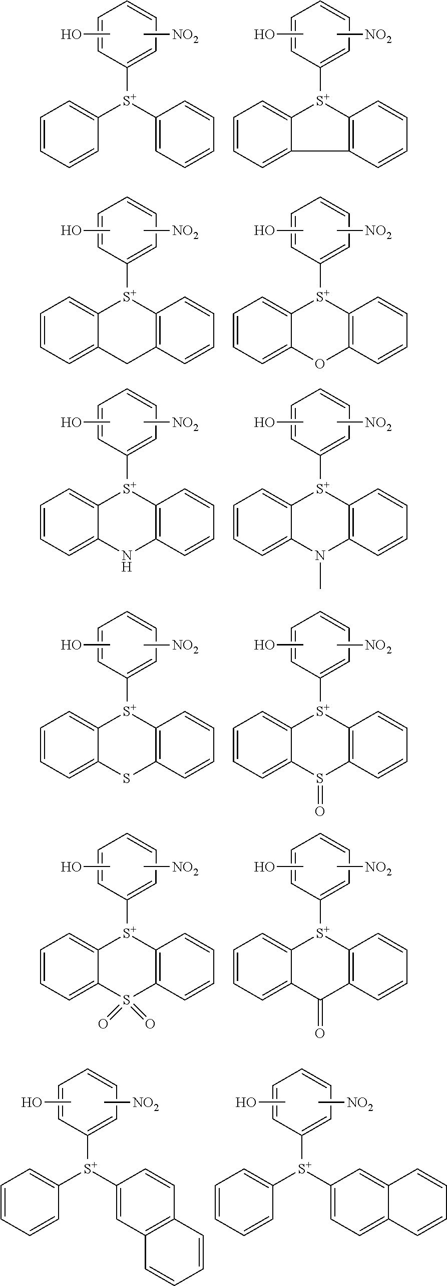

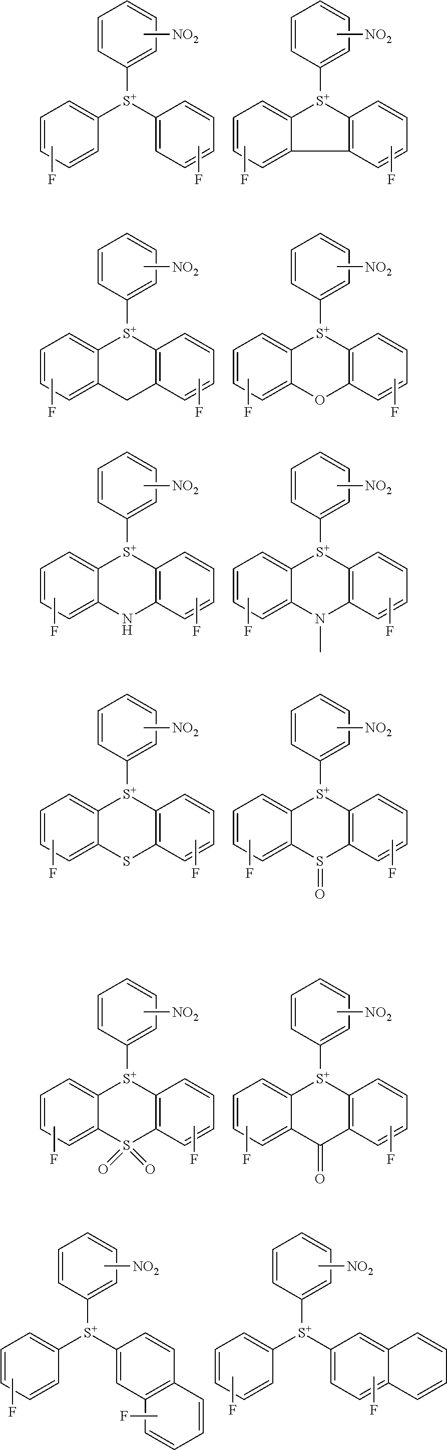

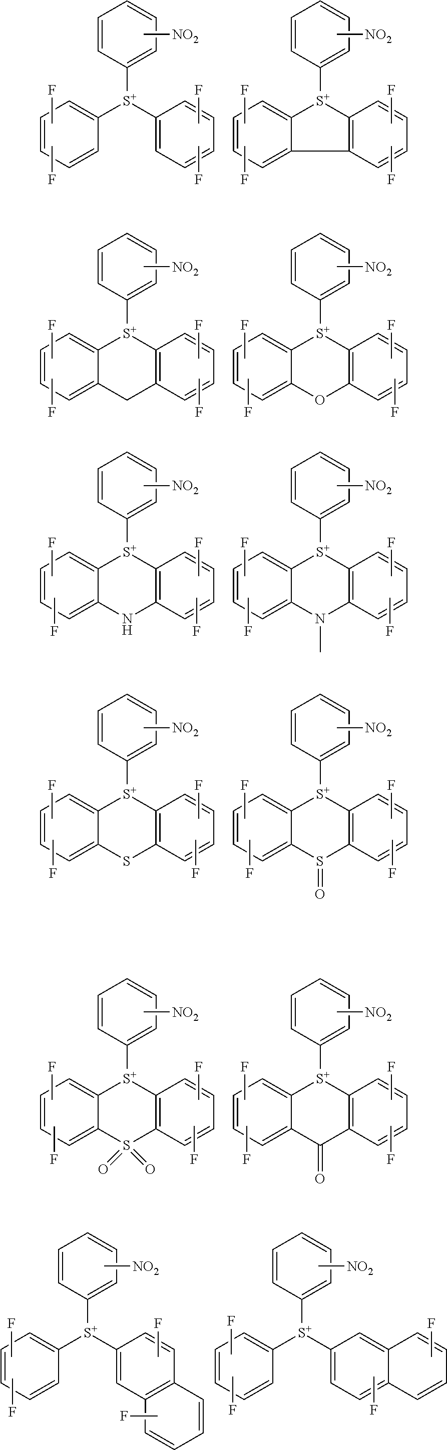

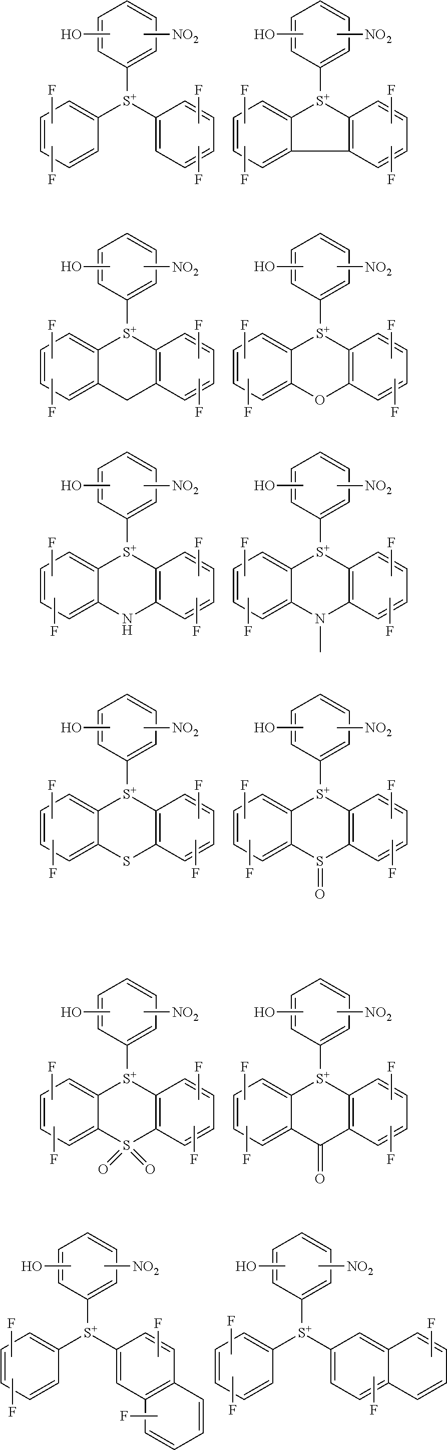

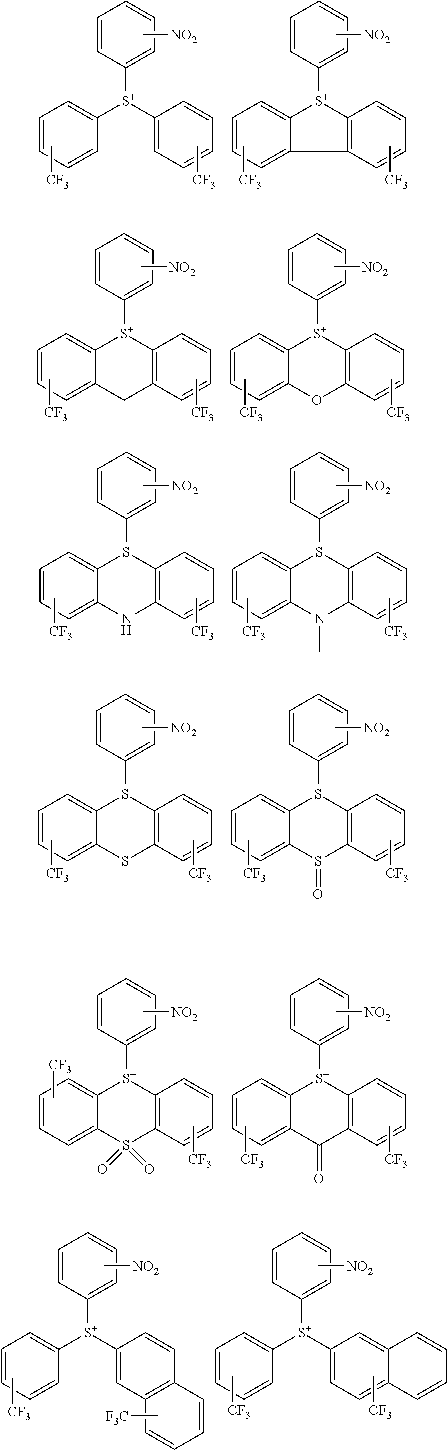

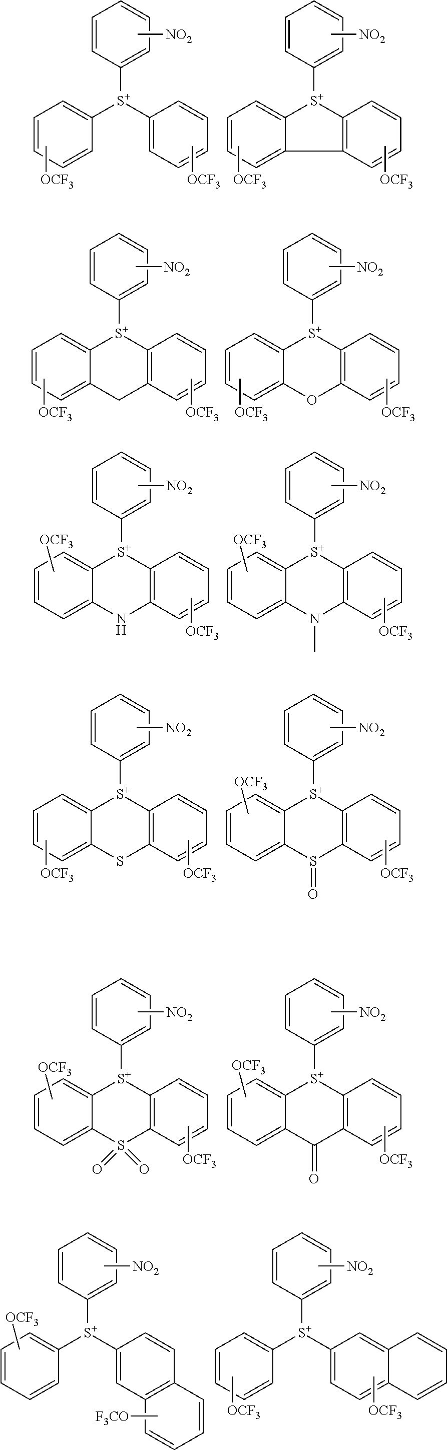

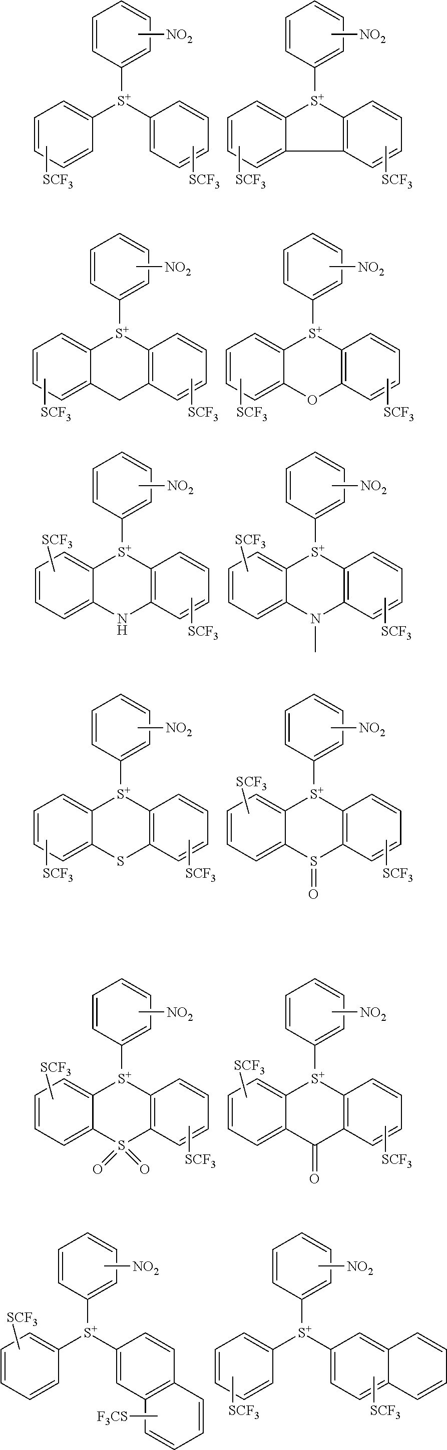

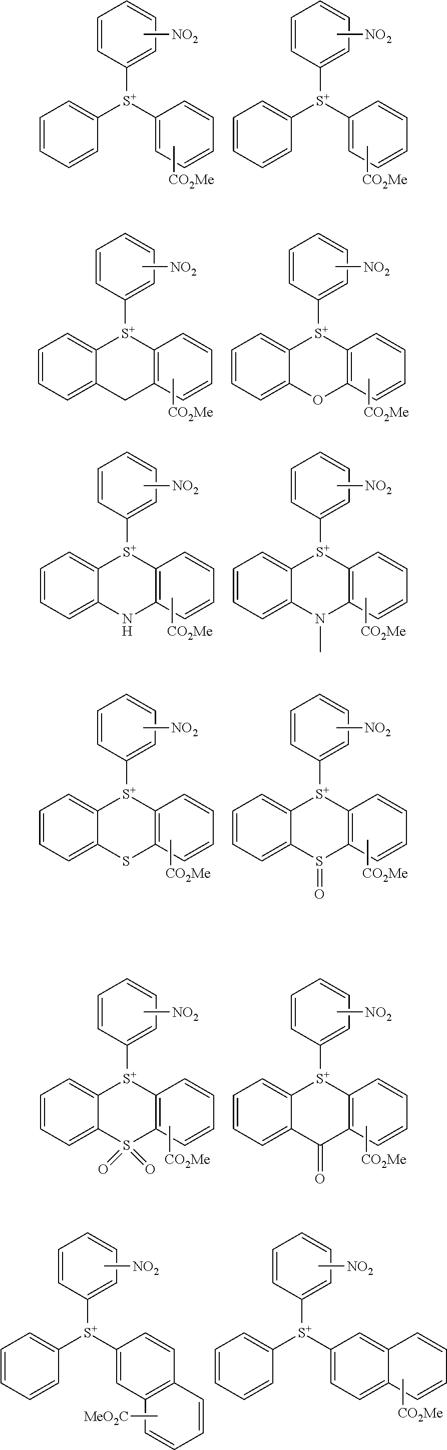

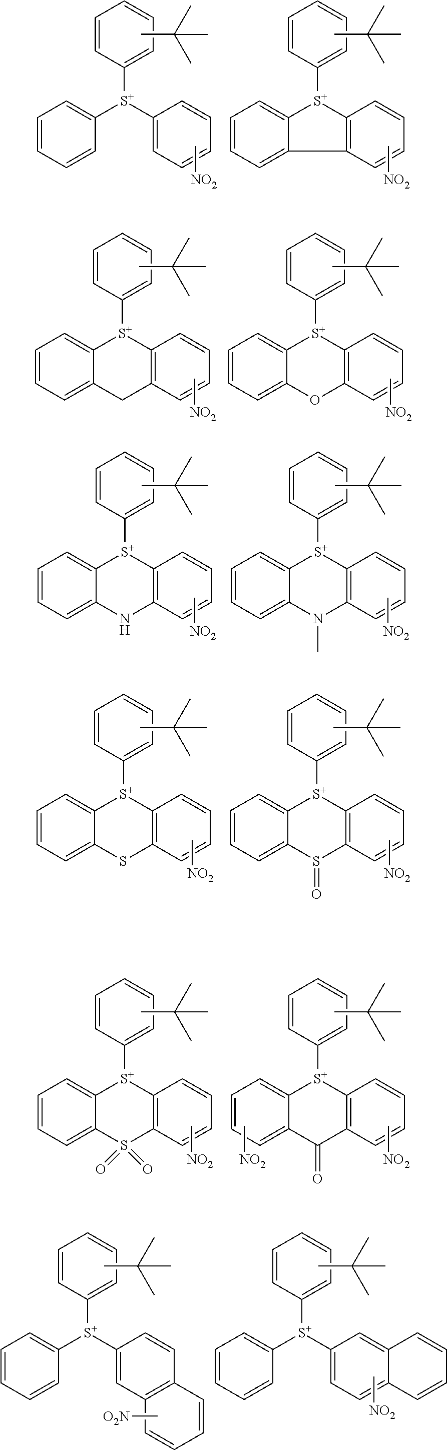

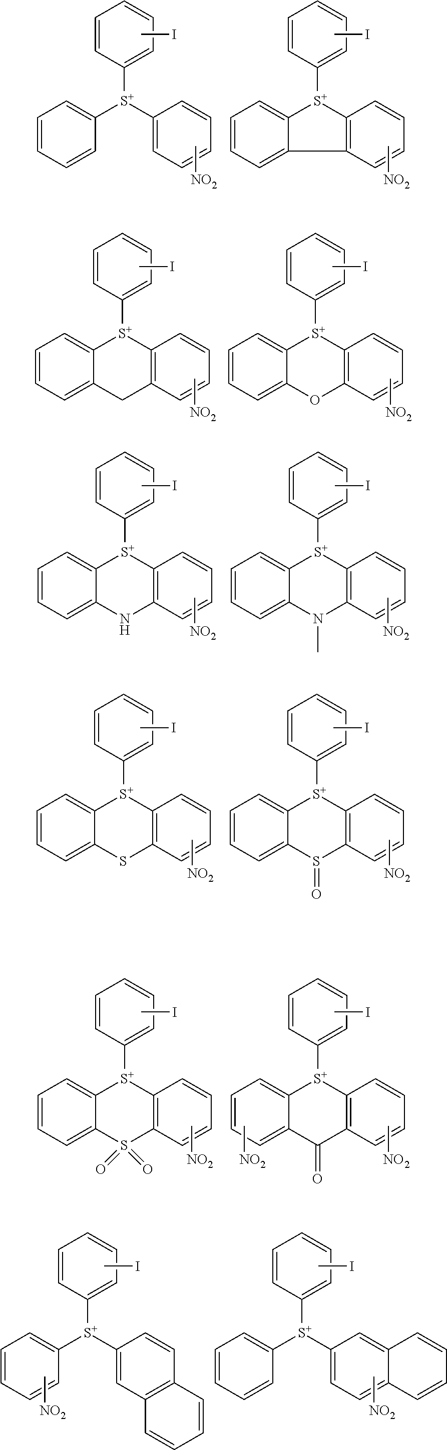

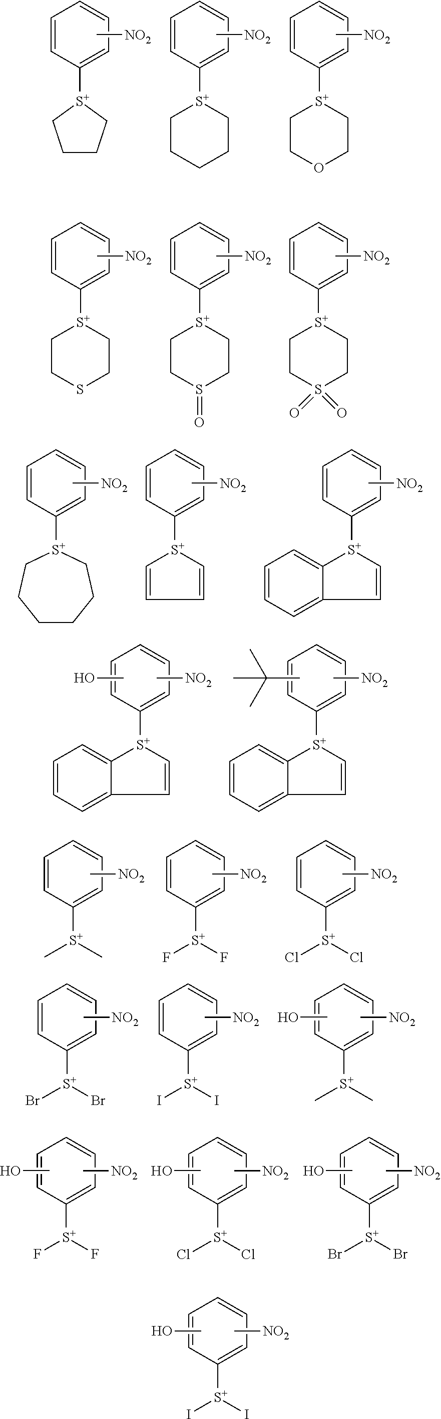

In the formula (A), Z⁻ is a fluoroalkanesulfonate anion having an aromatic vinyl structure and an iodine atom. As the fluoroalkanesulfonate anion, a fluoroalkanesulfonate anion having the formula (Z) is preferred.

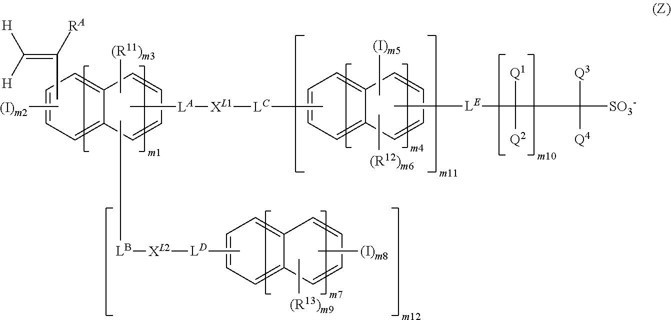

In the formula (Z), m1 is 0 or 1. The relevant structure is a benzene ring in case of m1=0, and a naphthalene ring in case of m1=1. The benzene ring corresponding to m1=0 is preferred from the aspect of solvent solubility. m2 is 0, 1, 2, 3, or 4. m2 is preferably 0, 1, 2, or 3, more preferably 0, 1, or 2, and most preferably 0 or 1 from the aspect of availability of reactants. m3 is 0, 1, 2, or 3.

In the formula (Z), m4 is 0 or 1. The relevant structure is a benzene ring in case of m4=0, and a naphthalene ring in case of m4=1. The benzene ring corresponding to m4=0 is preferred from the aspect of solvent solubility. m5 is 0, 1, 2, 3, or 4. m5 is preferably 0, 1, 2, or 3, more preferably 0, 1, or 2 from the aspect of availability of reactants. m6 is 0, 1, 2, or 3.

In the formula (Z), m7 is 0 or 1. The relevant structure is a benzene ring in case of m7=0, and a naphthalene ring in case of m7=1. The benzene ring corresponding to m7=0 is preferred from the aspect of solvent solubility. m8 is 1, 2, 3, or 4. m8 is preferably 1, 2, or 3, more preferably 1, or 2 from the aspect of availability of reactants. m9 is 0, 1, 2, or 3.

In the formula (Z), m10 is 0, 1, 2, 3, or 4, but is preferably 0, 1, 2, or 3, more preferably 1, 2, or 3, most preferably 1. m11 is 0 or 1. m12 is 0 or 1.

It is noted that 0≤m2+m3+m12≤4 in case of m1=0 and 0≤m2+m3+m12≤6 in case of m1=1; 0≤m5+m6≤4 in case of m4-0 and 0≤m5+m6≤6 in case of m4=1; and 0≤m8+m9≤5 in case of m7-0 and 0≤m8+m9≤7 in case of m7=1. An anion containing more iodine atoms is more absorptive to EUV, but loses solvent solubility so that it may precipitate in a resist composition, 1≤m2+m5+m8≤4 is preferred.

In the formula (Z), R^{A} is hydrogen, fluorine, methyl or trifluoromethyl. R^{A} is preferably hydrogen or methyl, more preferably hydrogen.

In the formula (Z), R¹¹ is halogen exclusive of iodine, nitro, cyano, hydroxy, carboxy, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom. The halogen exclusive of iodine is preferably fluorine, chlorine, or bromine, more preferably fluorine. The hydrocarbyl group and hydrocarbyl moiety in the hydrocarbyloxy and hydrocarbylthio groups may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, 1-propenyl, 2-propenyl, butenyl, and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl, and 2-phenylethyl; and combinations thereof. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety. A plurality of R¹¹ may be identical or different when m3 is 2 or 3.

When m3 is 2 or 3, two R¹¹ may bond together to form a ring with the carbon atoms to which they are attached. Examples of the ring thus formed include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

In the formula (Z), R¹² is halogen exclusive of iodine, nitro, cyano, hydroxy, carboxy, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom. Examples of the halogen exclusive of iodine include fluorine, chlorine, and bromine. The hydrocarbyl group and hydrocarbyl moiety in the hydrocarbyloxy and hydrocarbylthio groups may be saturated or unsaturated and straight, branched or cyclic. Examples thereof include those exemplified above for the hydrocarbyl group represented by R¹¹, but not limited thereto. A plurality of R¹² may be identical or different when m6 is 2 or 3.

When m6 is 2 or 3, two R¹² may bond together to form a ring with the carbon atoms to which they are attached. The ring is preferably a 5- to 8-membered ring.

In the formula (Z), R¹³ is halogen exclusive of iodine, nitro, cyano, hydroxy, carboxy, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom. Examples of the halogen exclusive of iodine include fluorine, chlorine, and bromine. The hydrocarbyl group and hydrocarbyl moiety in the hydrocarbyloxy and hydrocarbylthio groups may be saturated or unsaturated and straight, branched or cyclic. Examples thereof include those exemplified above for the hydrocarbyl group represented by R¹¹, but not limited thereto. A plurality of R¹³ may be identical or different when m9 is 2 or 3.

When m9 is 2 or 3, two R¹³ may bond together to form a ring with the carbon atoms to which they are attached. The ring is preferably a 5- to 8-membered ring.

In the formula (Z), L^{A}, L^{B}, L^{C}, L^{D}, and L^{E} are each independently a single bond, ether bond, ester bond, sulfonate ester bond, amide bond, sulfonamide bond, carbonate bond or carbamate bond. Of these, L^{A} is preferably a single bond, ether bond, ester bond or sulfonate ester bond, more preferably an ester bond or sulfonate ester bond. L^{B} is preferably a single bond, ether bond, ester bond, amide bond, sulfonamide bond or sulfonate ester bond, more preferably an ester bond or sulfonate ester bond. L^{C} is preferably a single bond, ether bond, ester bond, amide bond or sulfonate ester bond, more preferably a single bond, ether bond or ester bond. L^{D} is preferably a single bond, ether bond, ester bond, amide bond or sulfonate ester bond, more preferably a single bond, ether bond or ester bond. L^{E} is preferably a single bond, ether bond, ester bond or sulfonate ester bond, more preferably a single bond, ether bond or ester bond.

When m12 is 1, L^{A} and L^{B} are preferably bonded to vicinal carbon atoms on the aromatic ring. At this time, since the substituent containing the fluorosulfonate anion structure and the substituent containing an aromatic ring substituted with iodine are present at more spatially close positions, higher sensitivity is expected.

In the formula (Z), X^{L1} and X^{L2} are each independently a single bond or a C₁-C₄₀ hydrocarbylene group which may contain a heteroatom. The hydrocarbylene group may be straight, branched or cyclic, and examples thereof include alkanediyl, cyclic saturated hydrocarbylene and arylene groups. Examples of the heteroatom include oxygen, nitrogen and sulfur atoms.

Examples of the C₁-C₄₀ hydrocarbylene group which may contain a heteroatom represented by X^{L1} and X^{L2} include those shown below, but not limited thereto. In the formula, the asterisk (*) is a point of attachment to L^{A} and L^{C} or L^{B} and L^{D}, respectively.

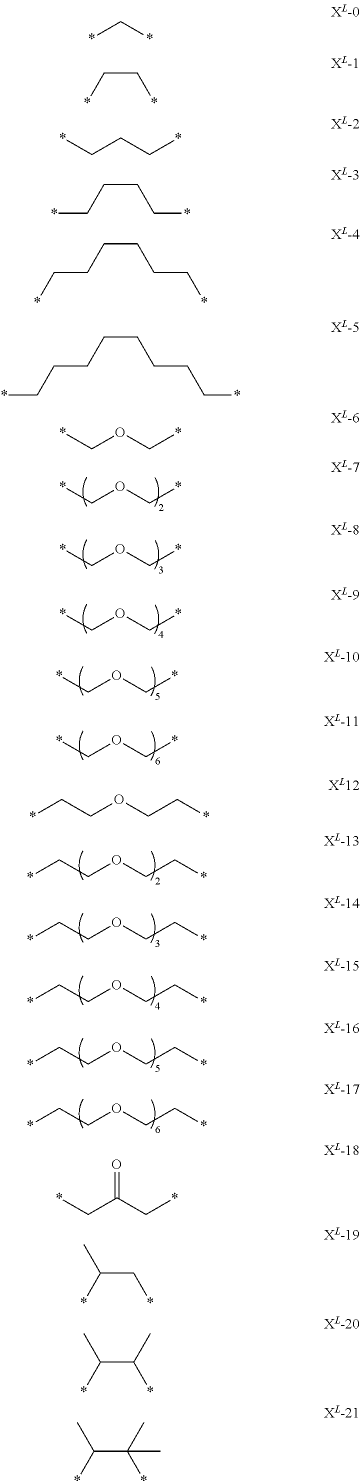

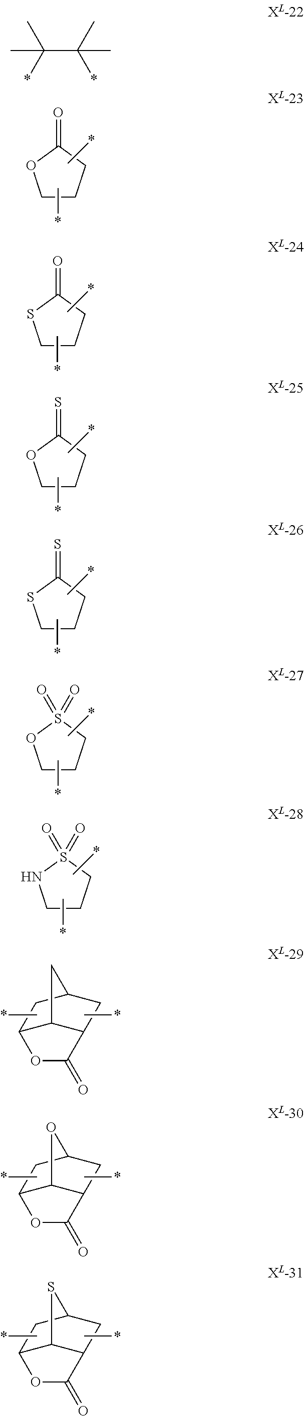

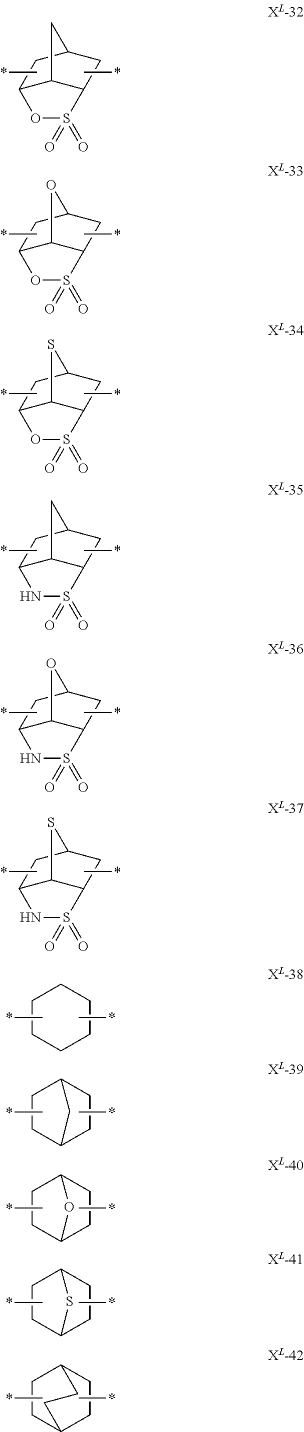

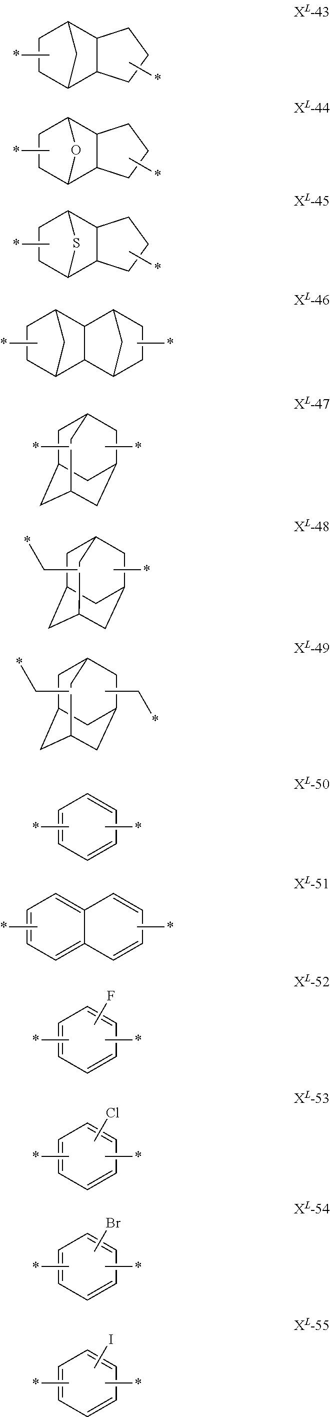

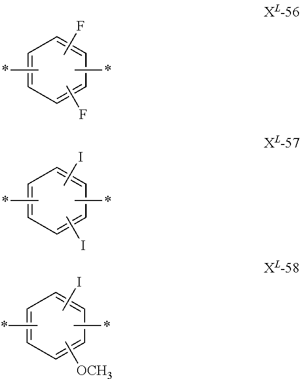

Of these, XL-0 to XL-22, XL-29 to XL-34, and XL-47 to XL-58 are preferred.

In the formula (Z), it is excluded that m11 and m12 are 0 at the same time, and excluded that L^{A}, L^{B}, L^{C}, L^{D} X^{L1} and X^{L2} are a single bond at the same time.

In the formula (Z), Q¹ and Q² are each independently hydrogen, fluorine, or a C₁-C₆ fluorinated saturated hydrocarbyl group. The C₁-C₆ fluorinated saturated hydrocarbyl group is preferably a trifluoromethyl group.

In the formula (Z), Q³ and Q⁴ are each independently fluorine or a C₁-C₆ fluorinated saturated hydrocarbyl group. The C₁-C₆ fluorinated saturated hydrocarbyl group is preferably a trifluoromethyl group. Q³ and Q⁴ are more preferably fluorine.

Examples of the partial structure of —[C(Q¹) (Q²)]ₙ₆—C(Q³) (Q⁴)—SO₃⁻; in the formula (Z) are preferably those shown below, but are not limited thereto. In the formula, the asterisk (*) designates a point of attachment to L^{E}.

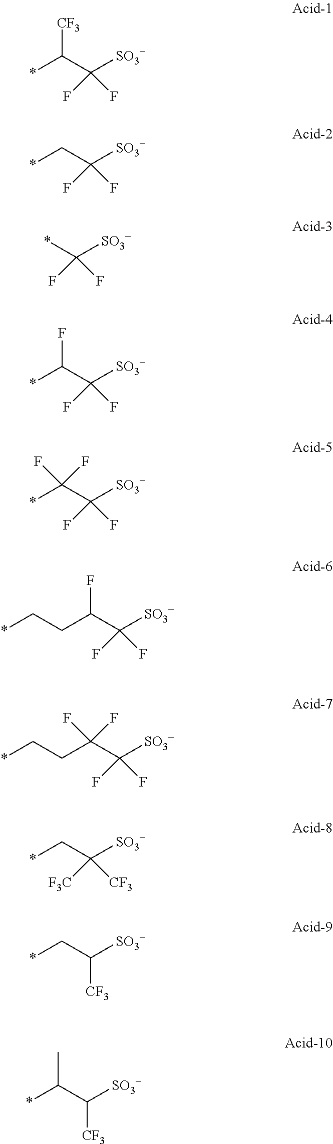

Of these, Acid-1 to Acid-7 are preferred, and Acid-1 to Acid-3, Acid-6, and Acid-7 are more preferred.

Examples of the sulfonium salt monomer anion having the formula (A) include those shown below, but not limited thereto. In the formula, R^{A} and Q¹ are as described above, and Me is methyl. The bond positions of substituents on the aromatic ring are interchangeable.

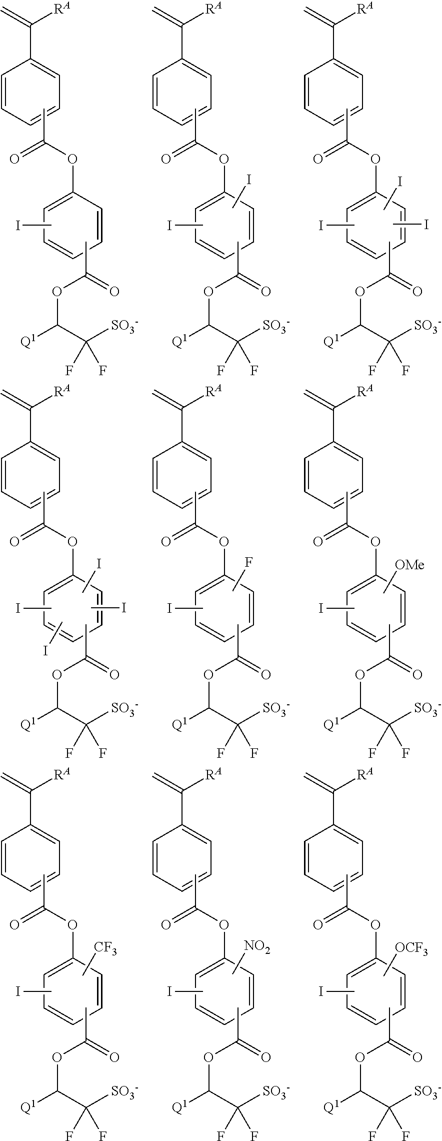

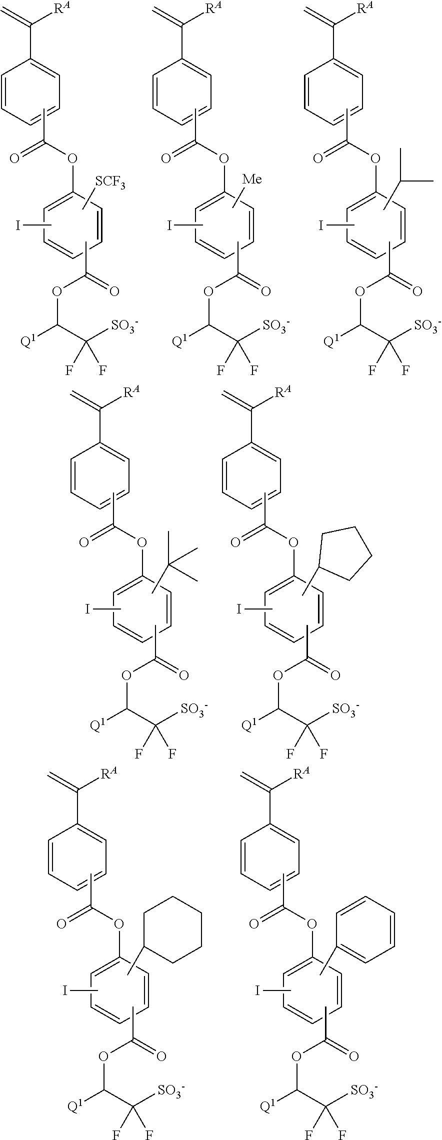

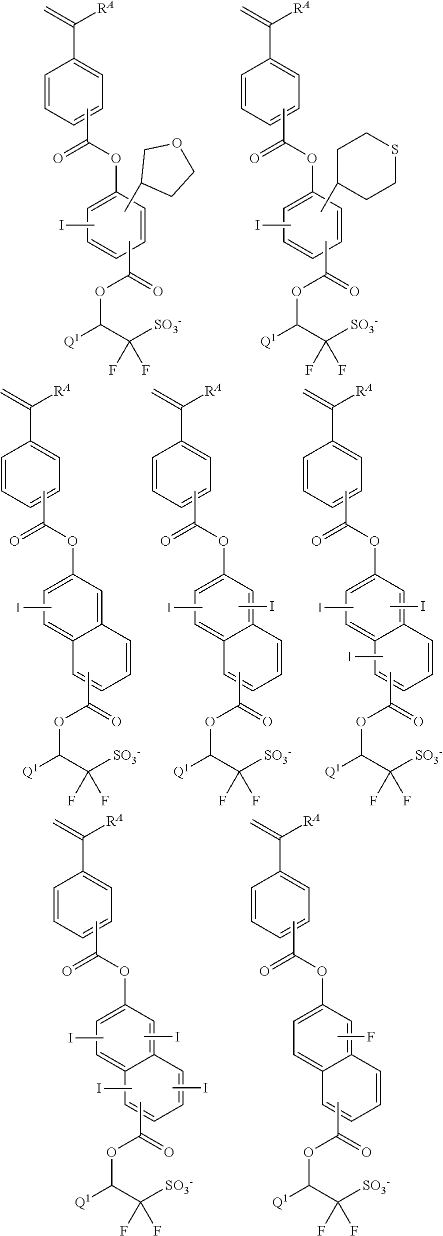

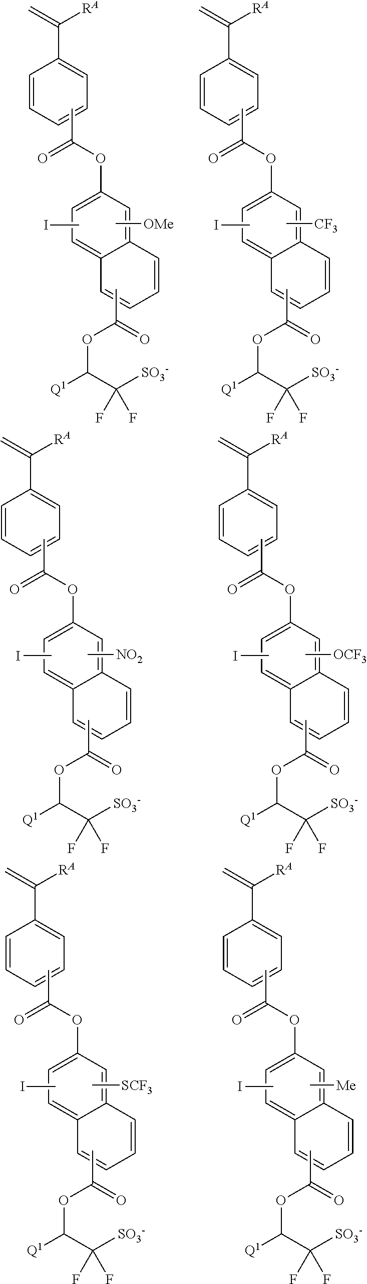

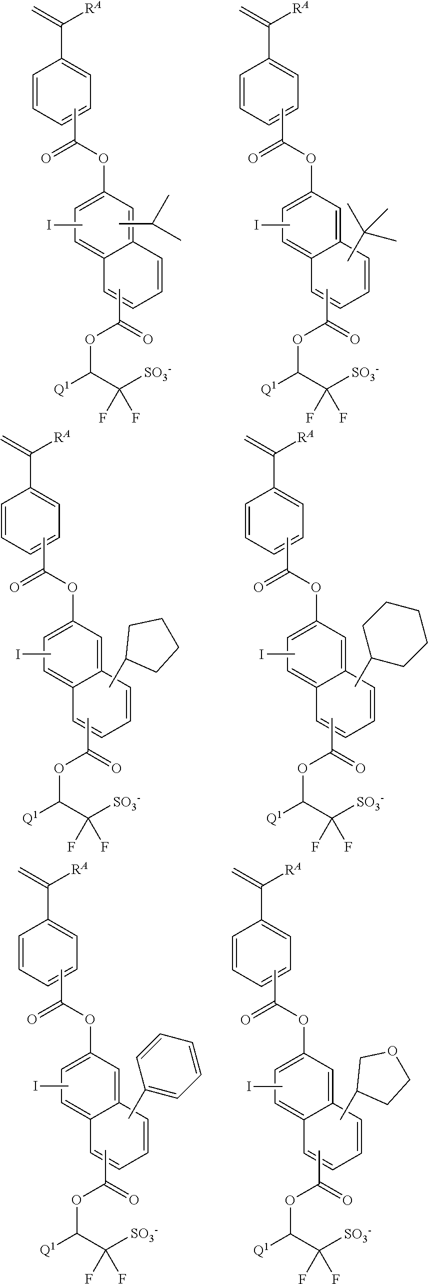

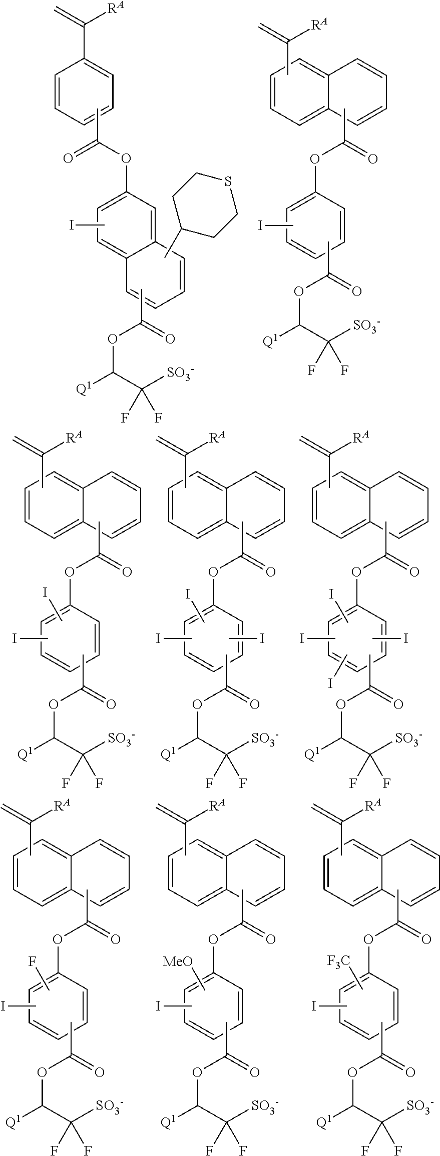

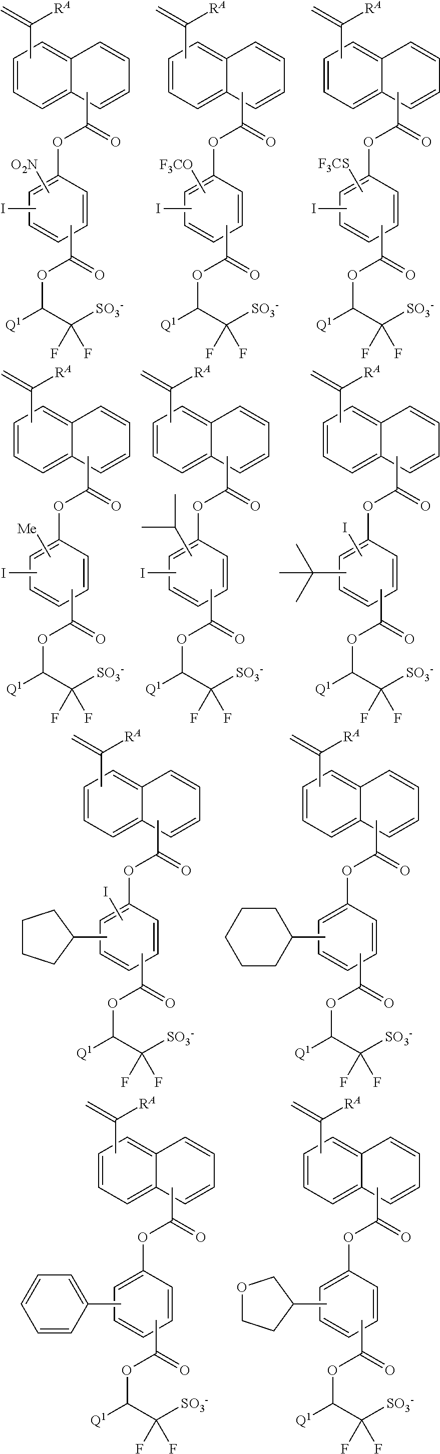

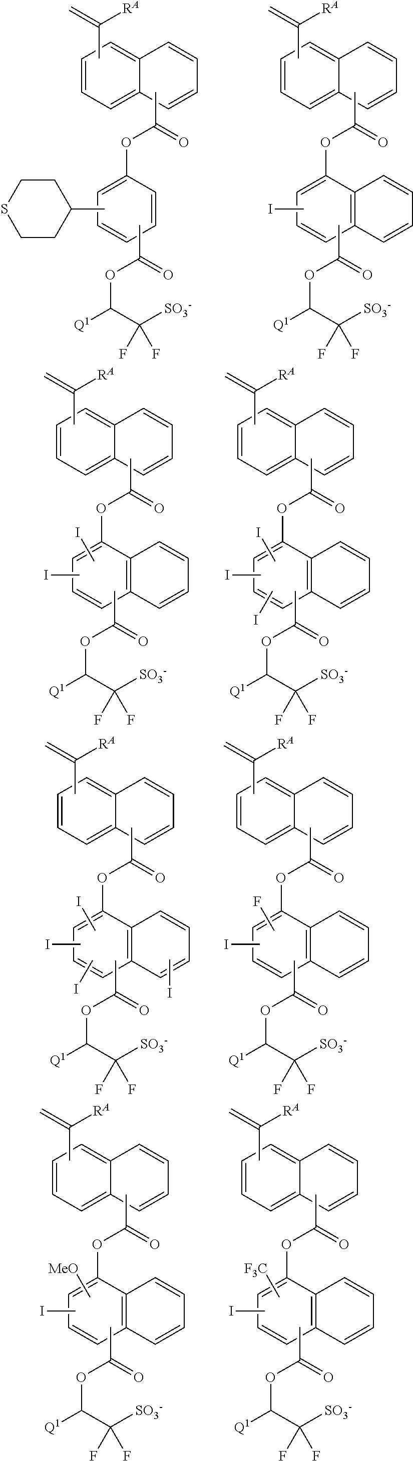

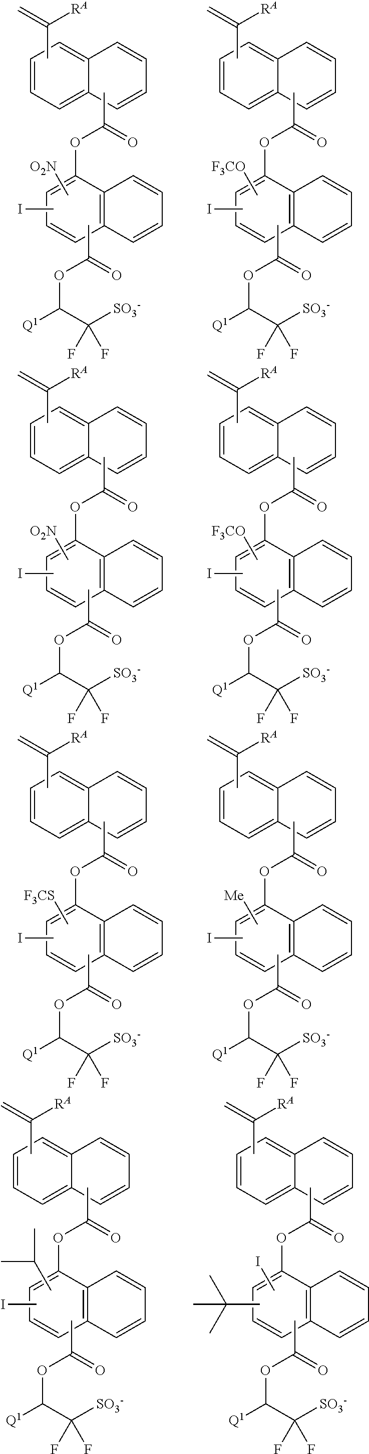

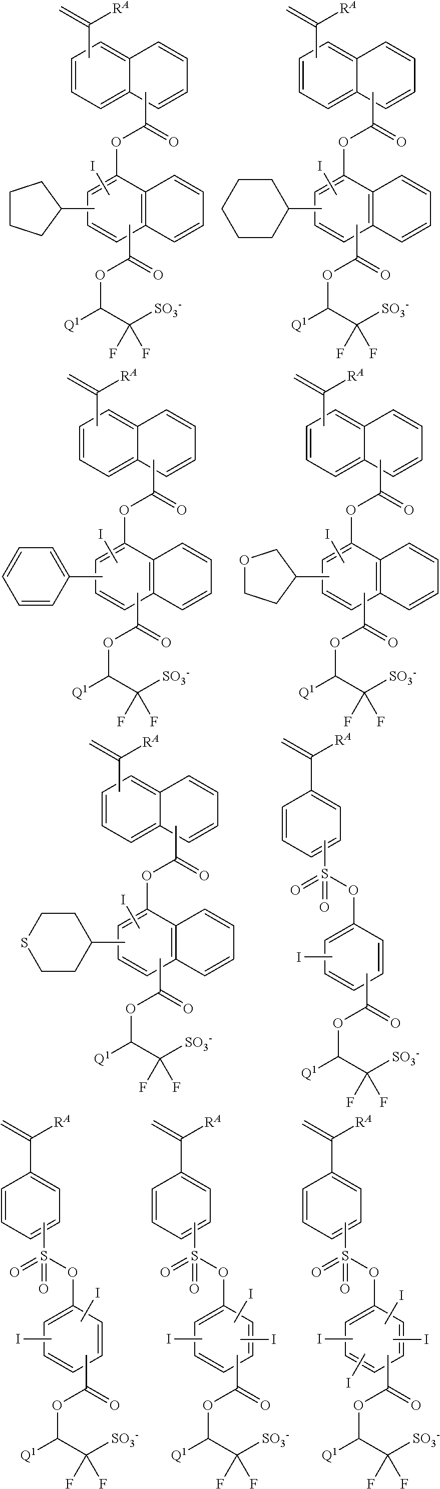

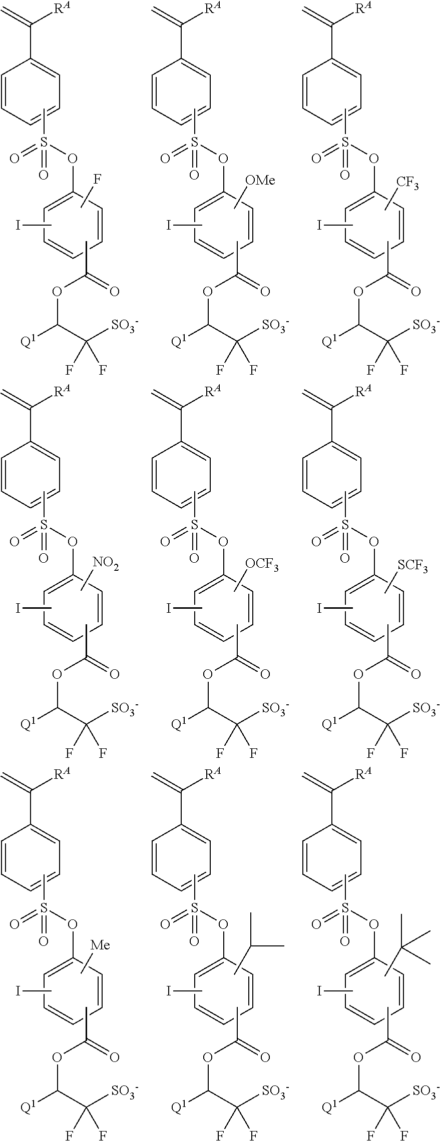

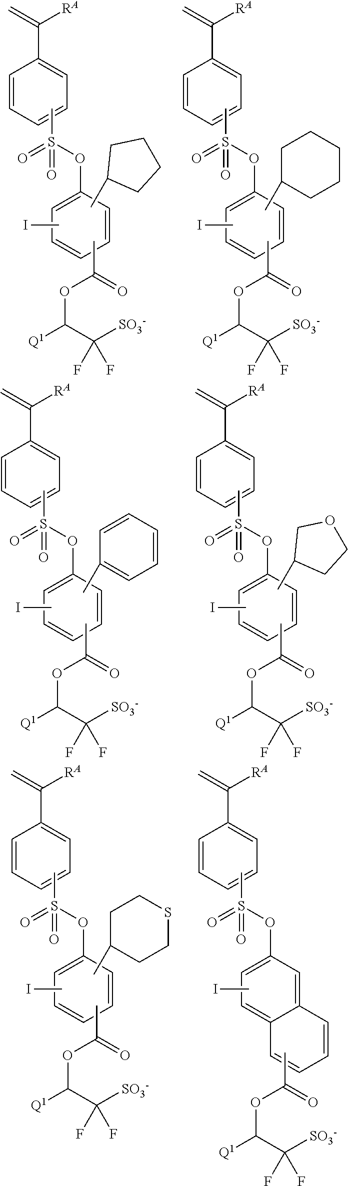

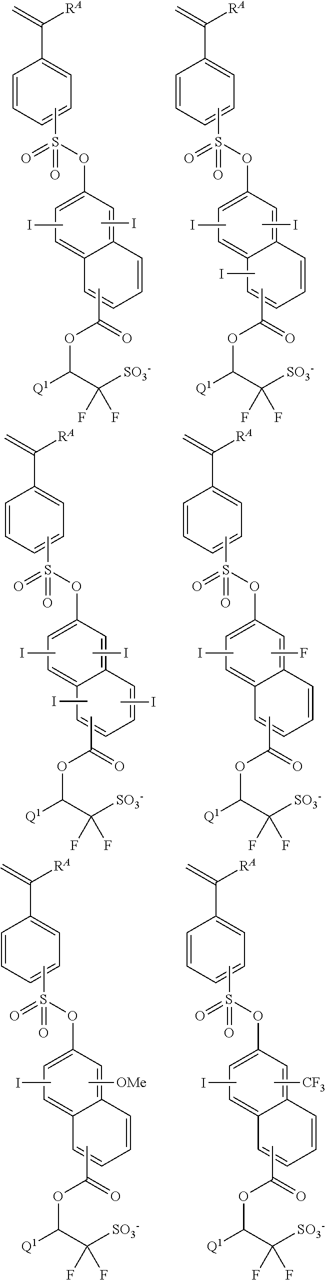

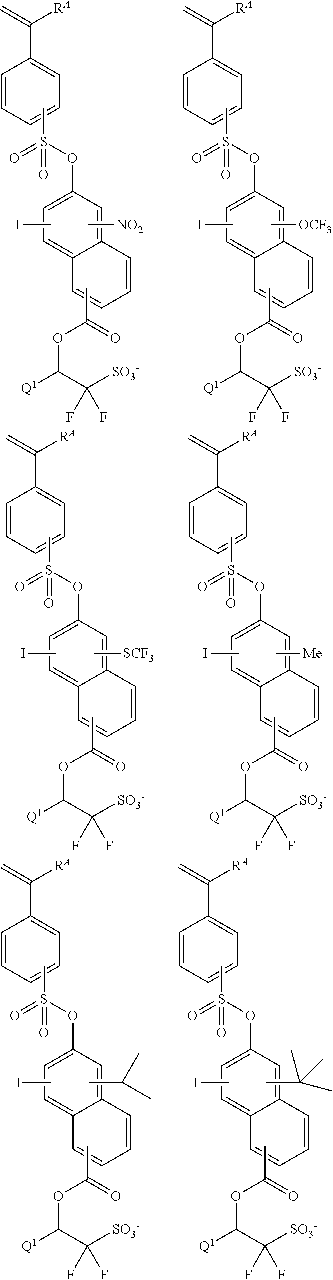

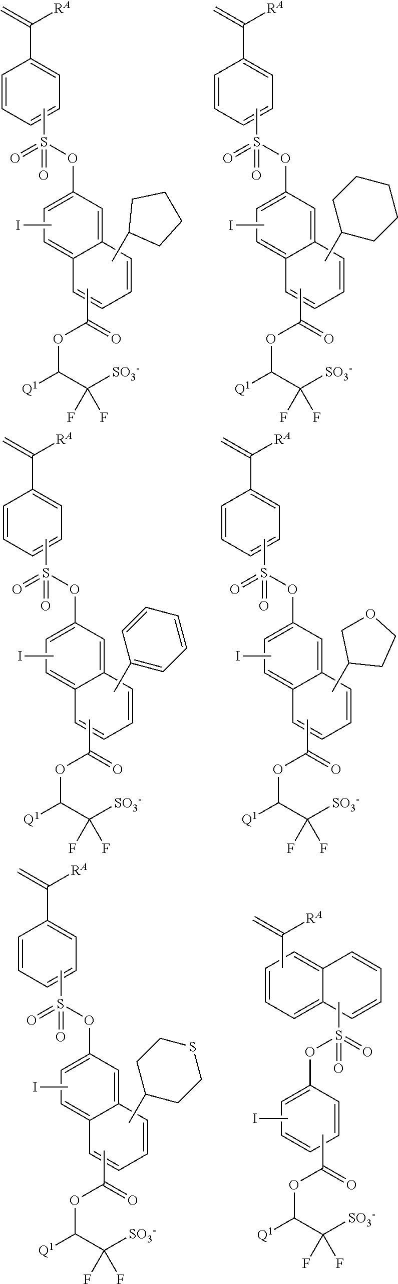

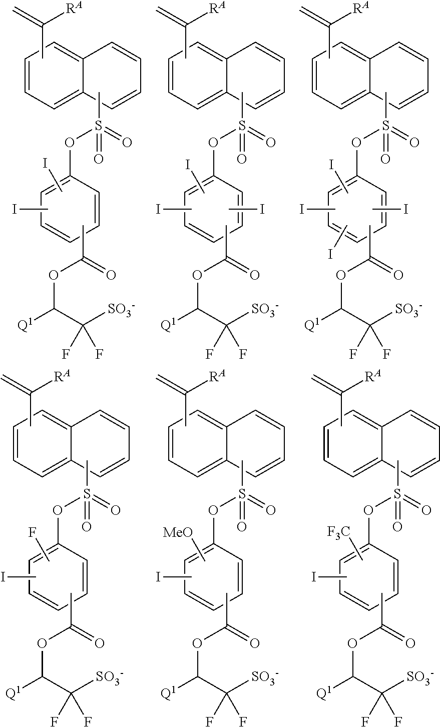

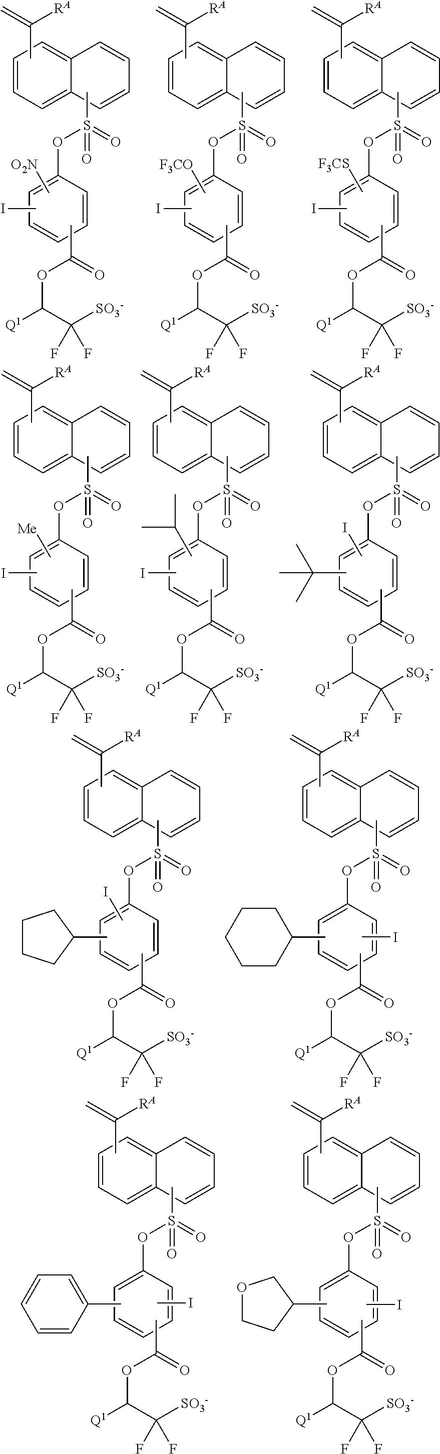

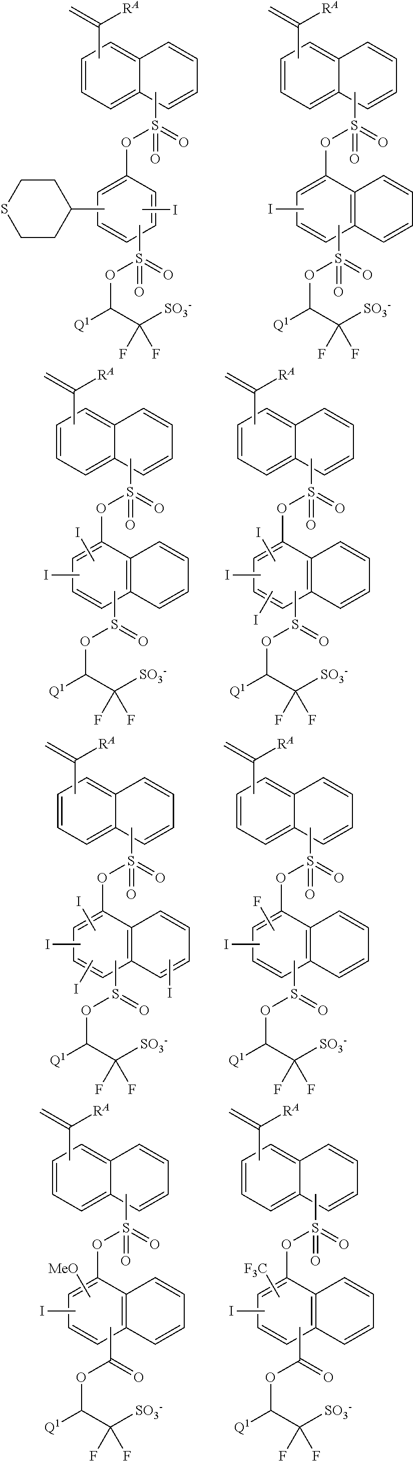

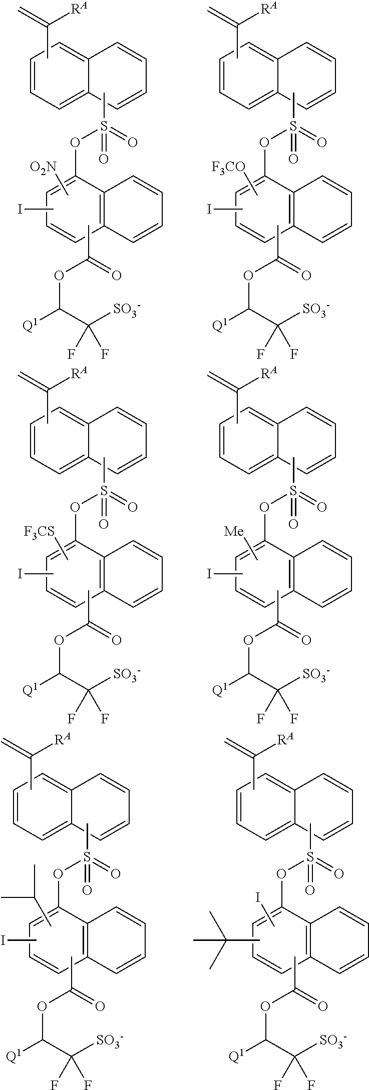

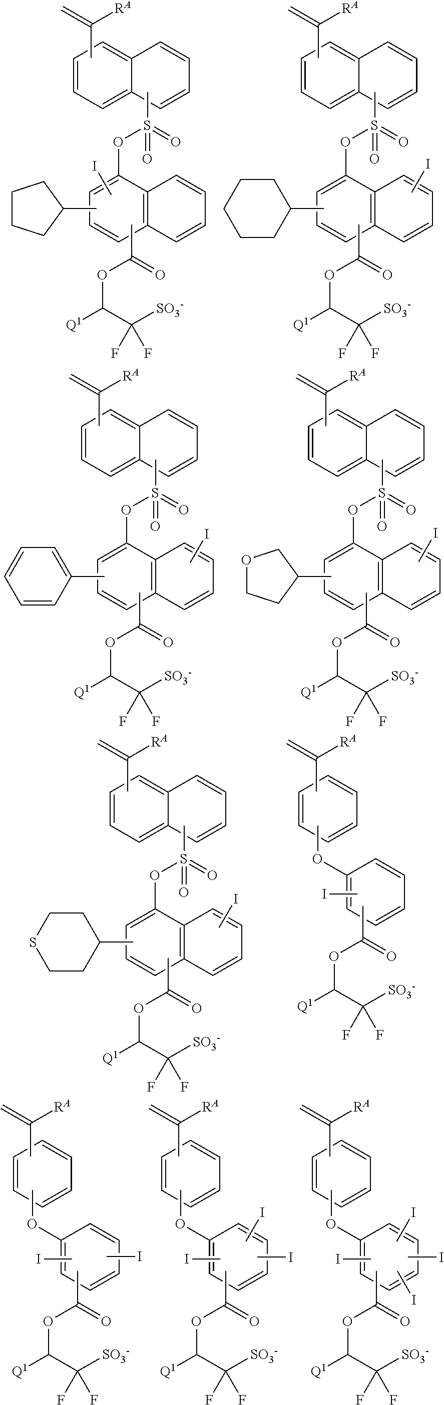

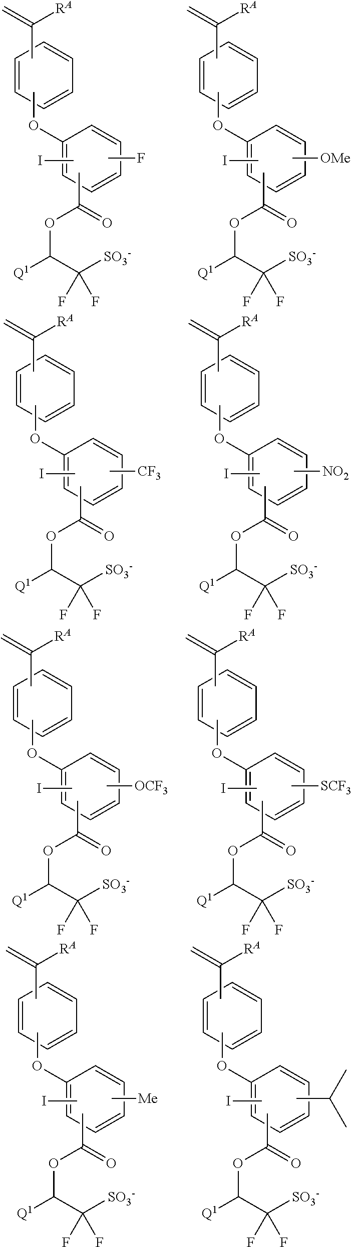

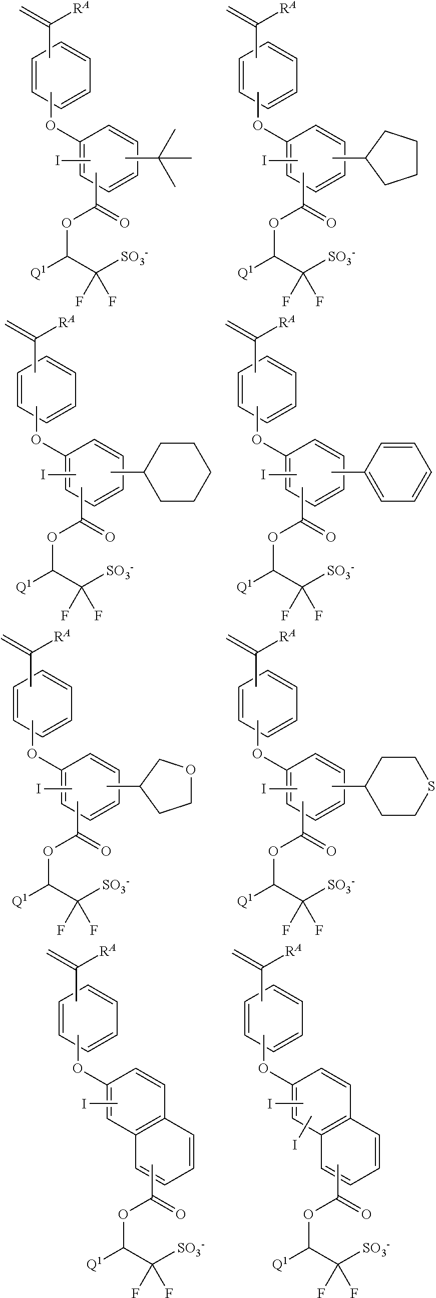

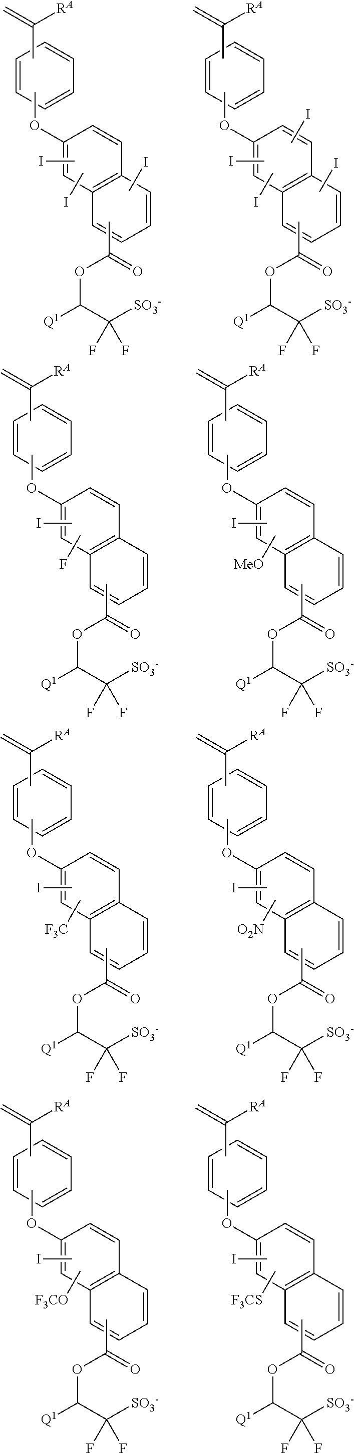

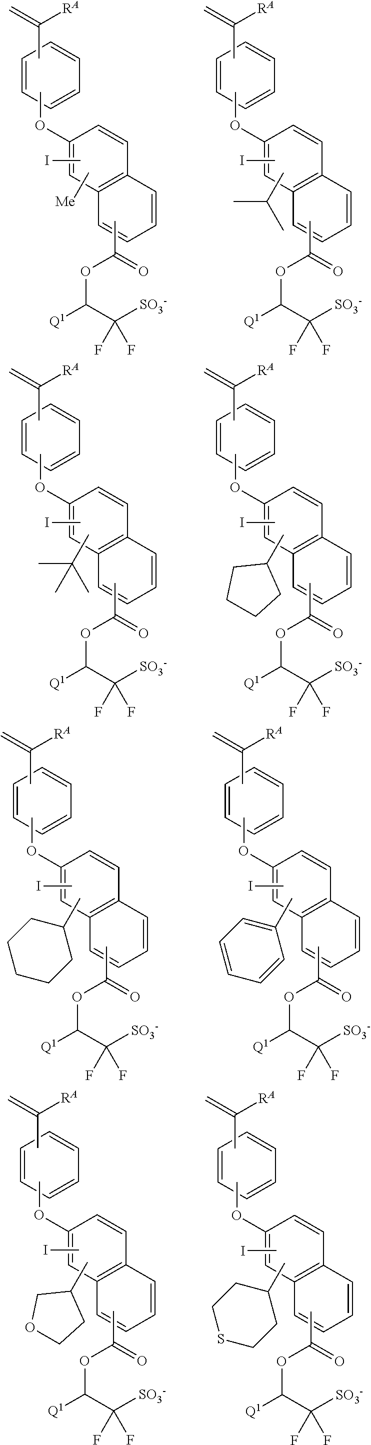

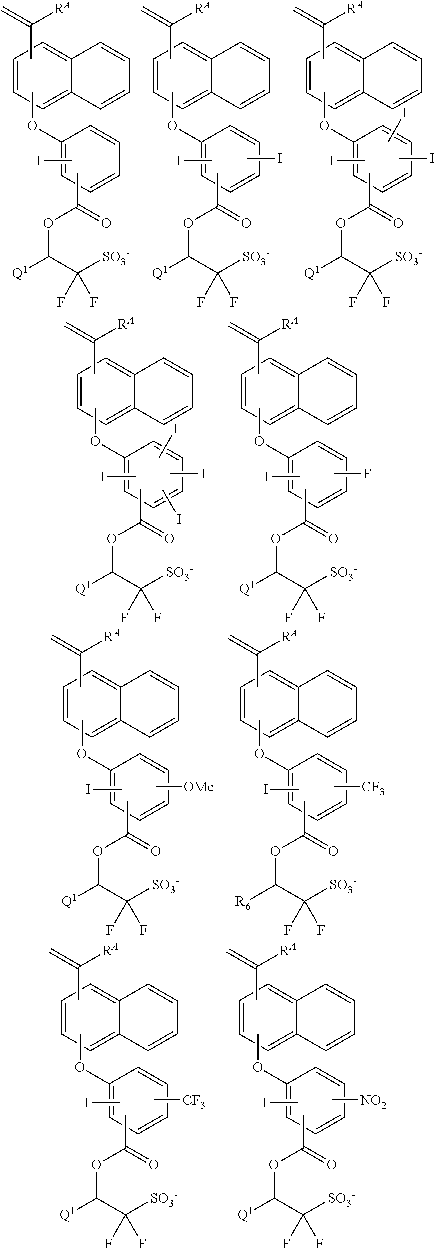

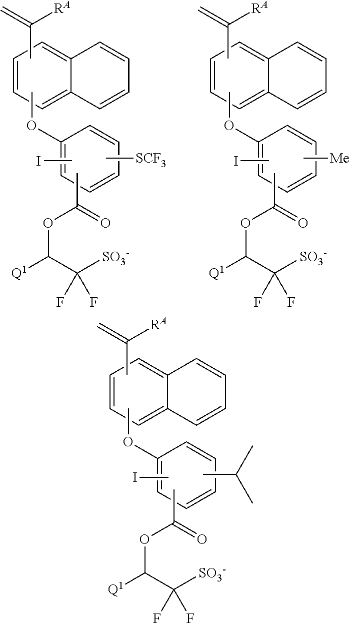

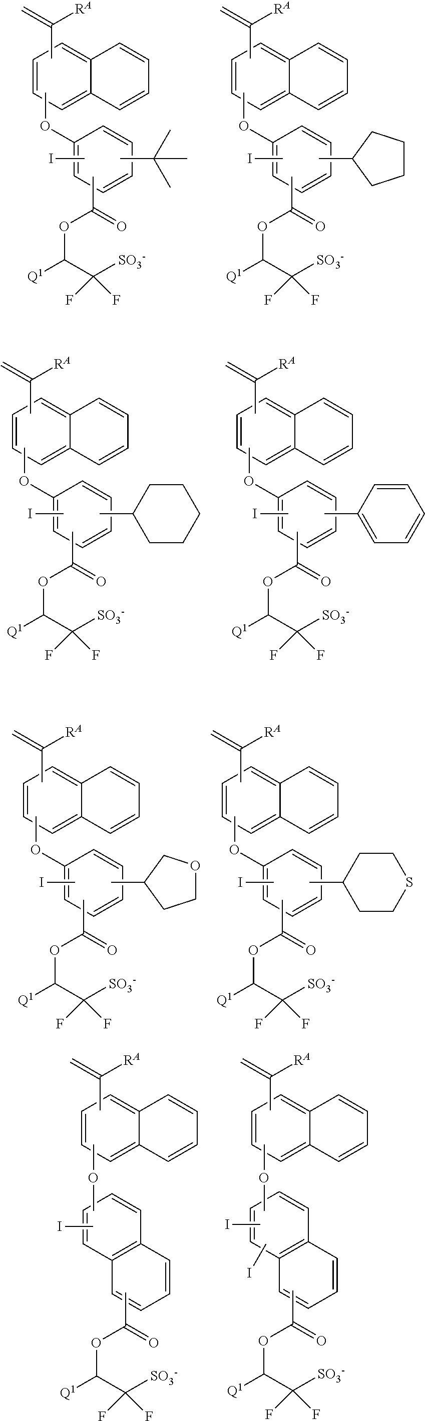

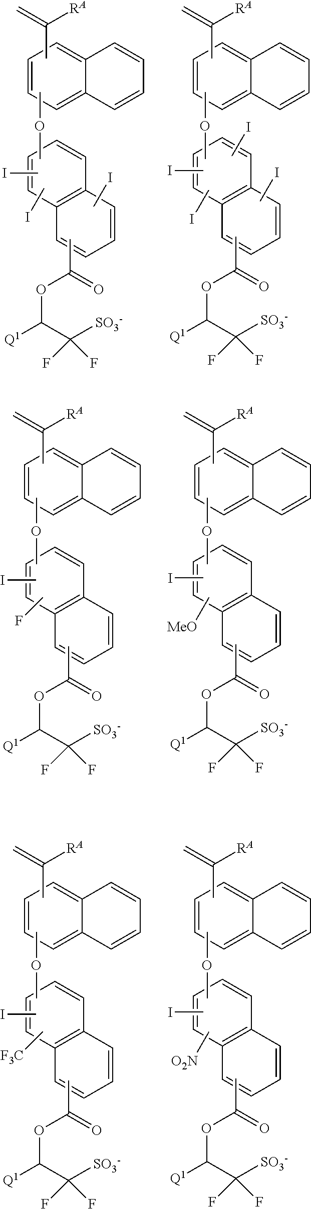

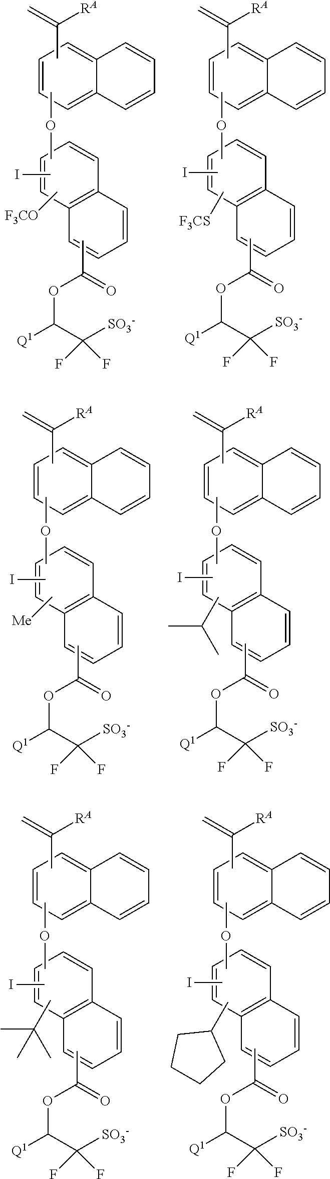

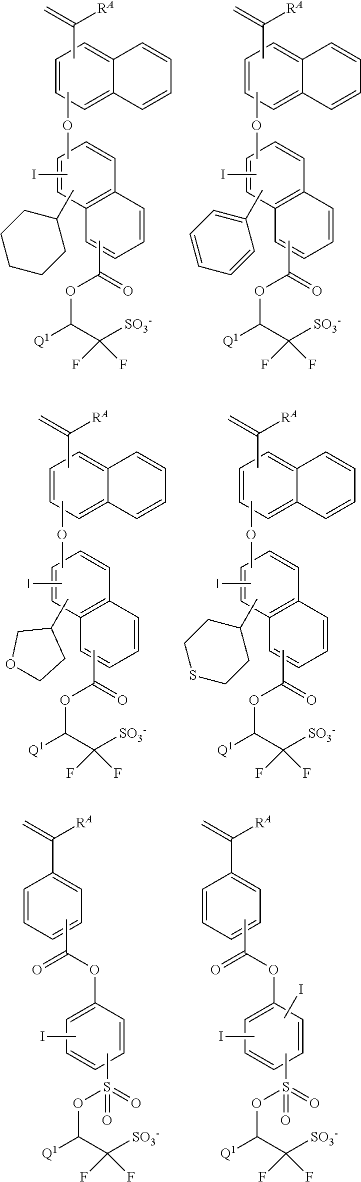

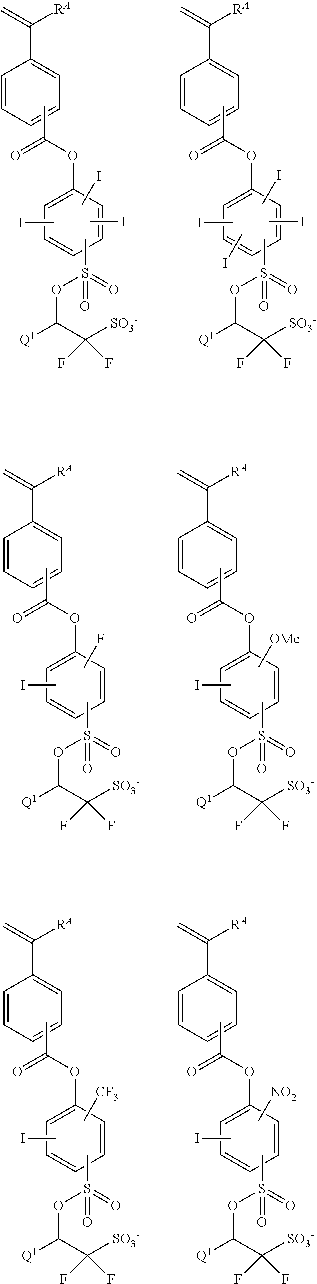

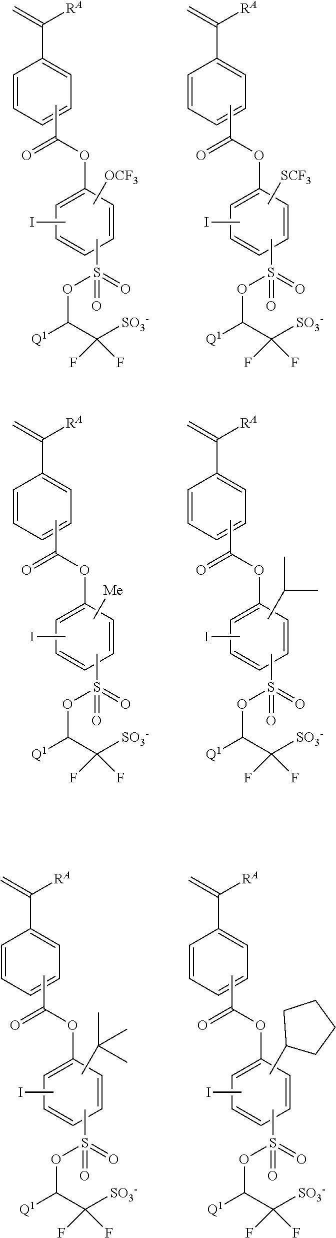

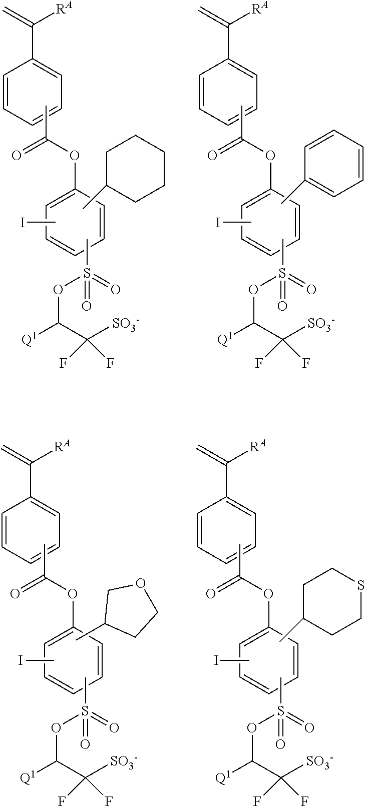

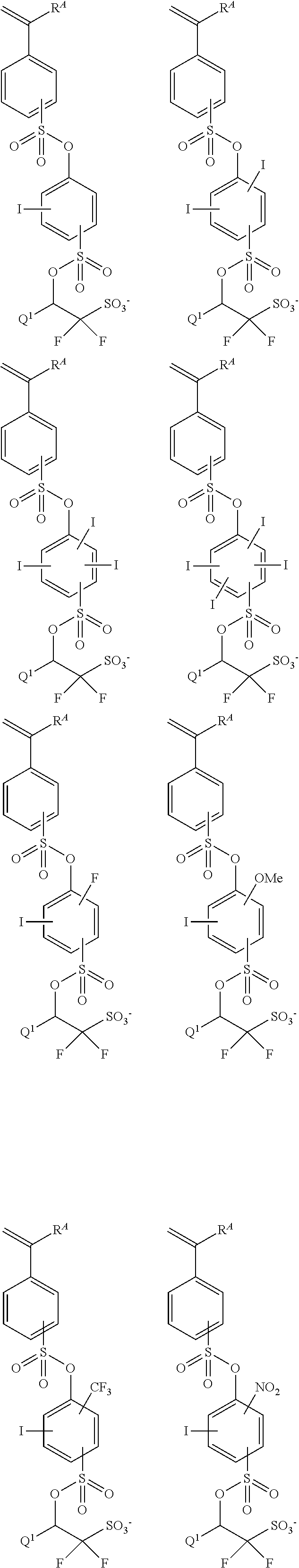

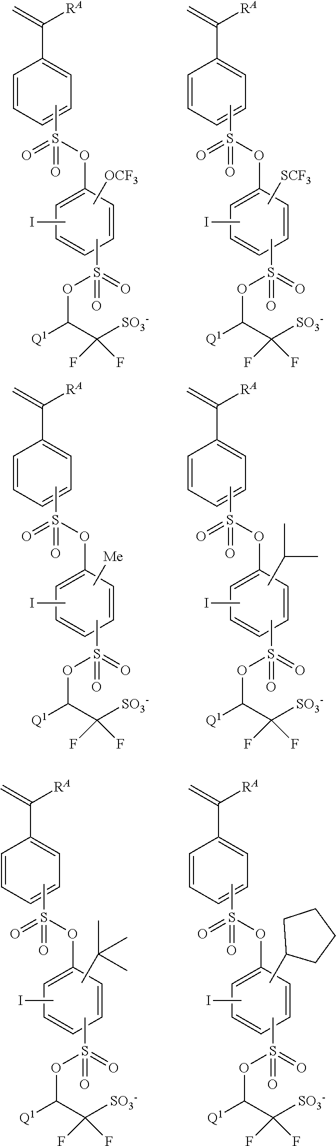

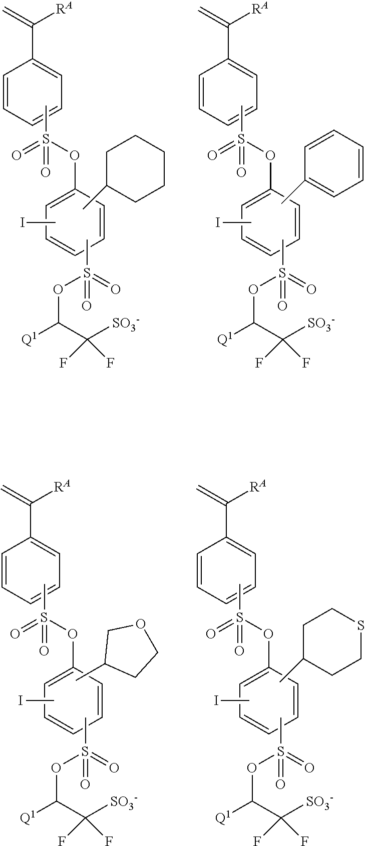

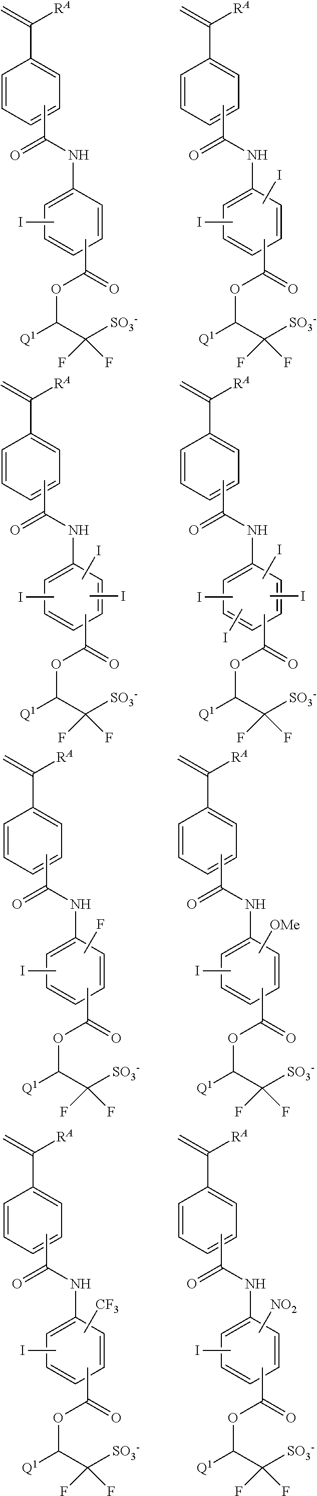

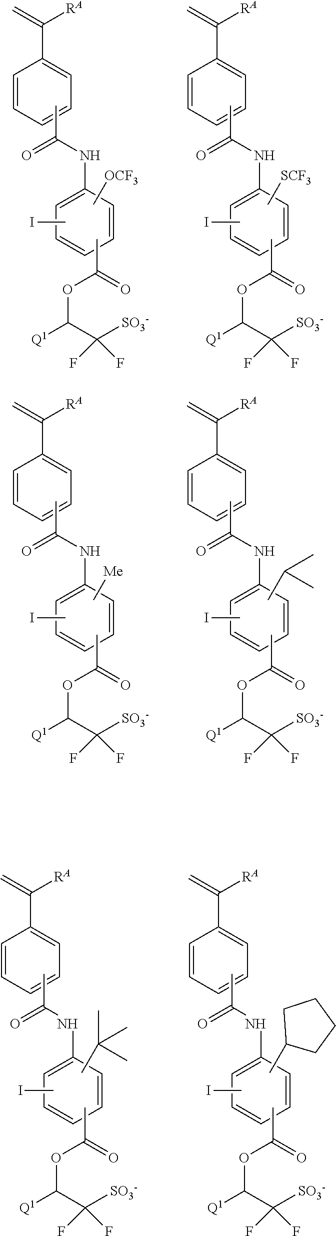

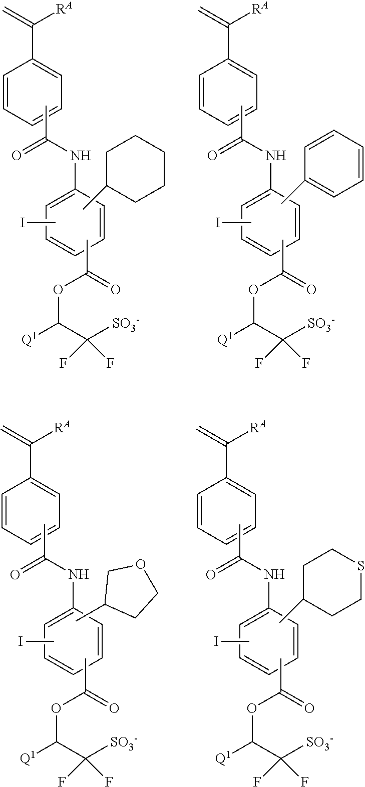

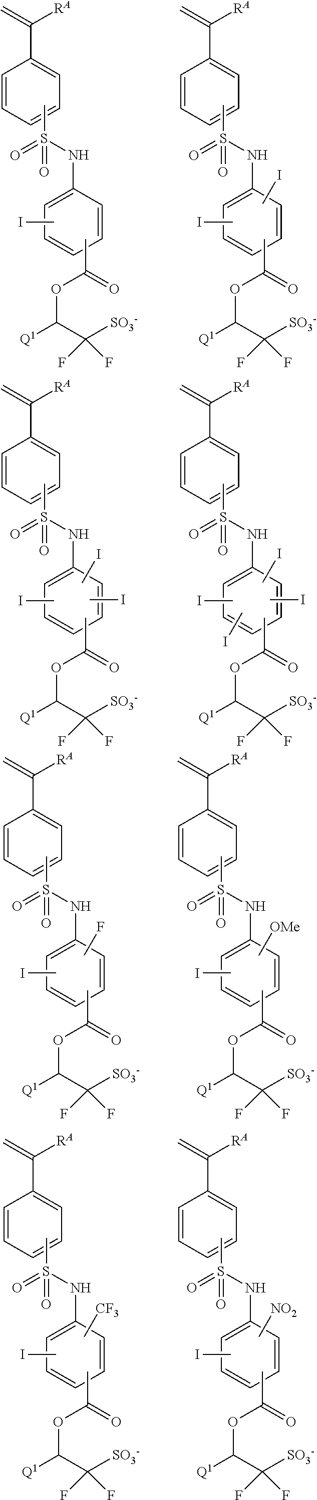

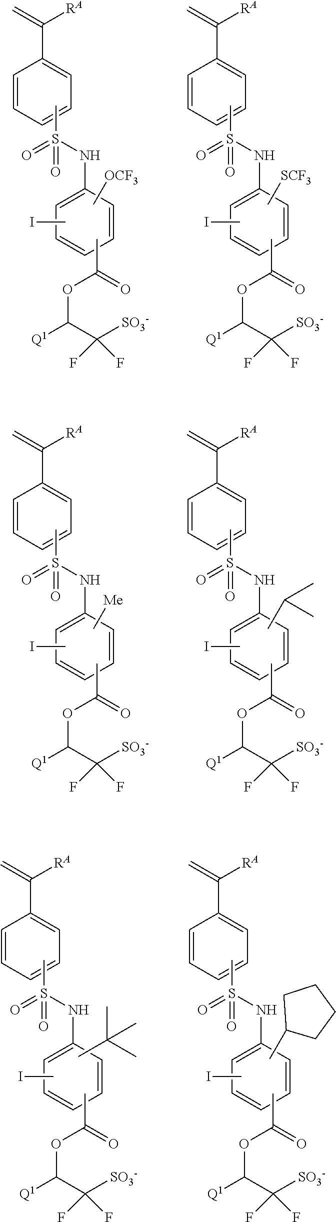

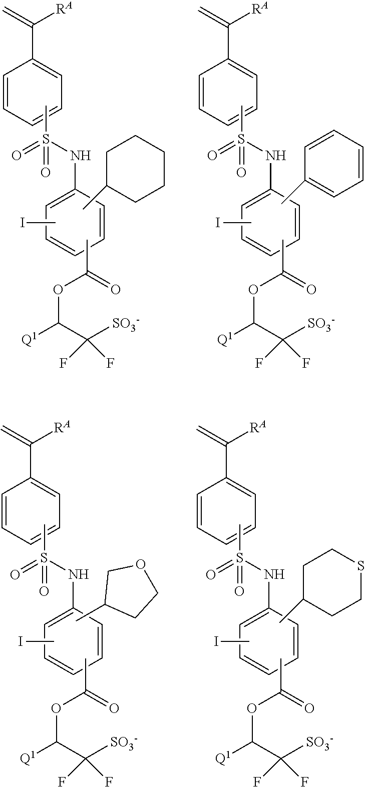

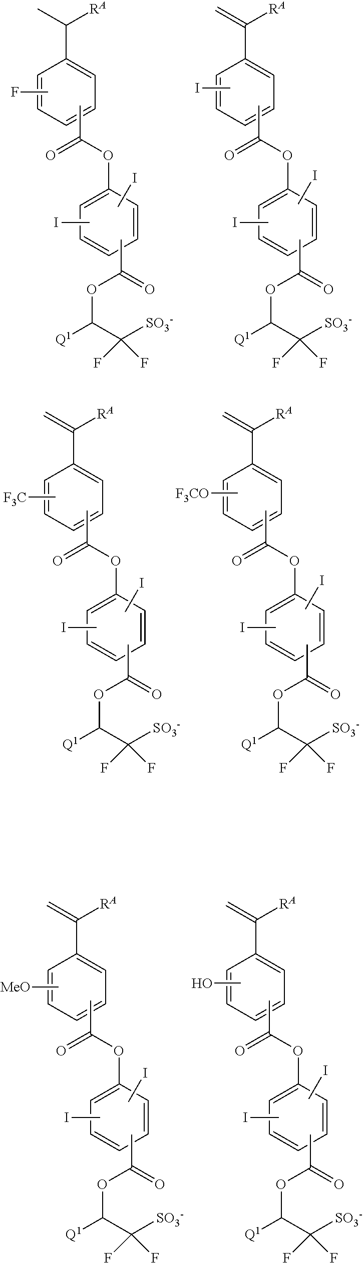

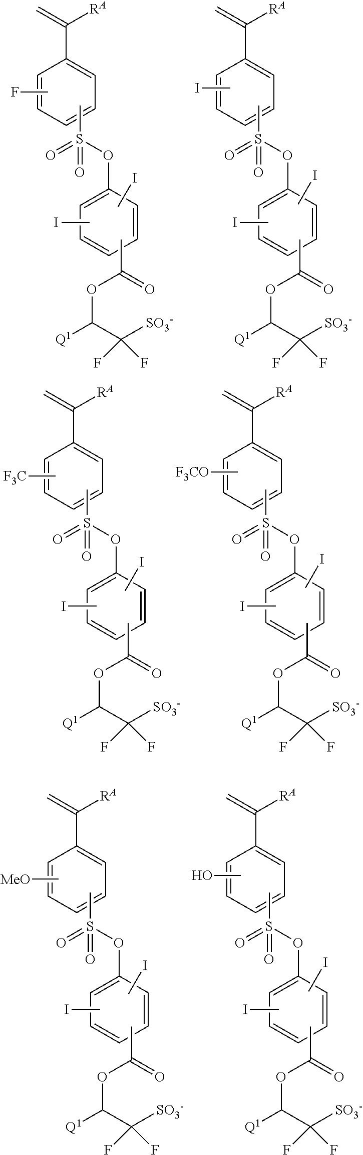

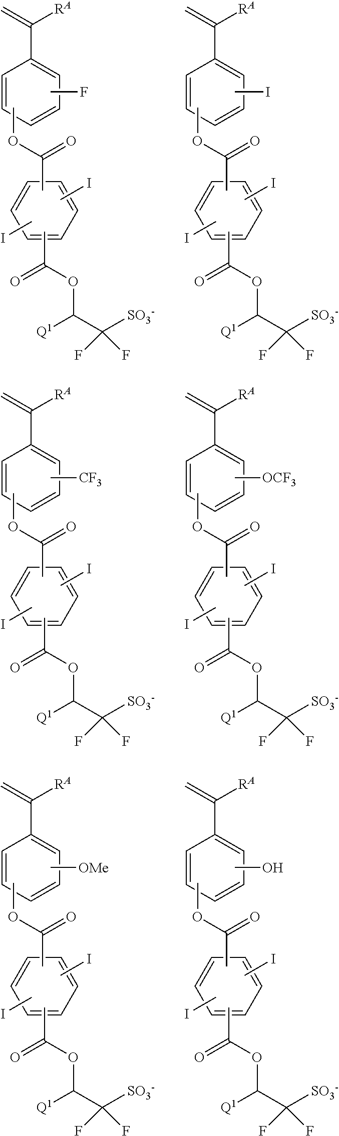

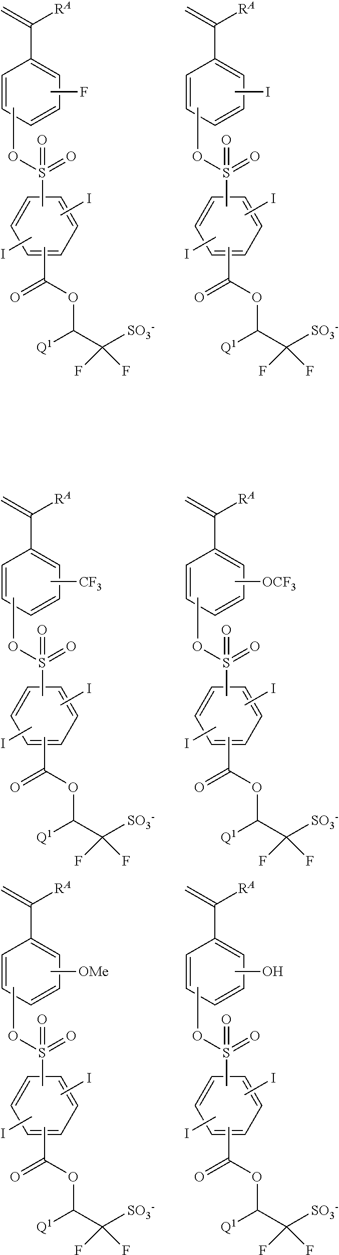

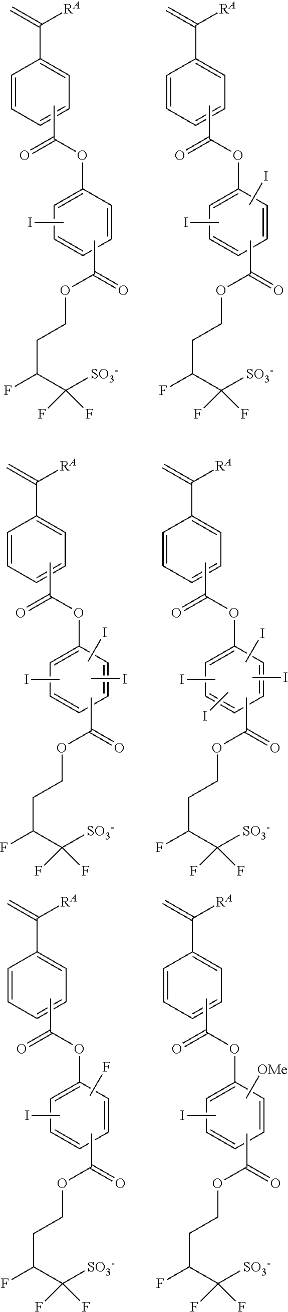

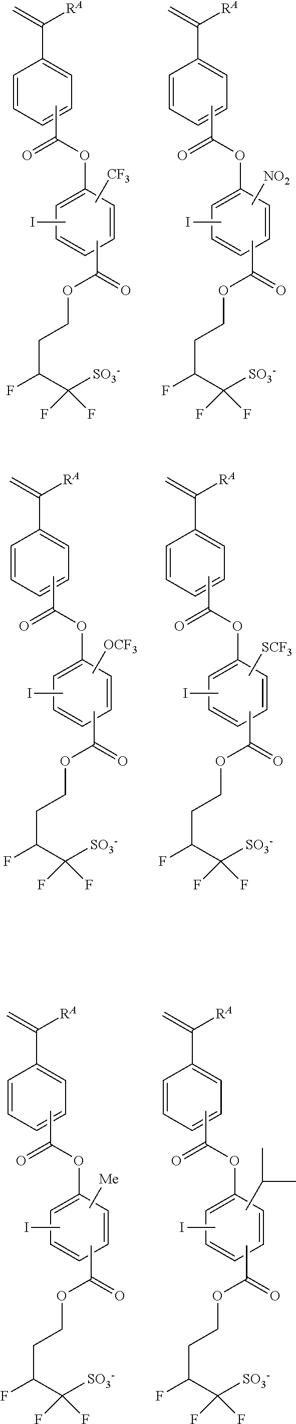

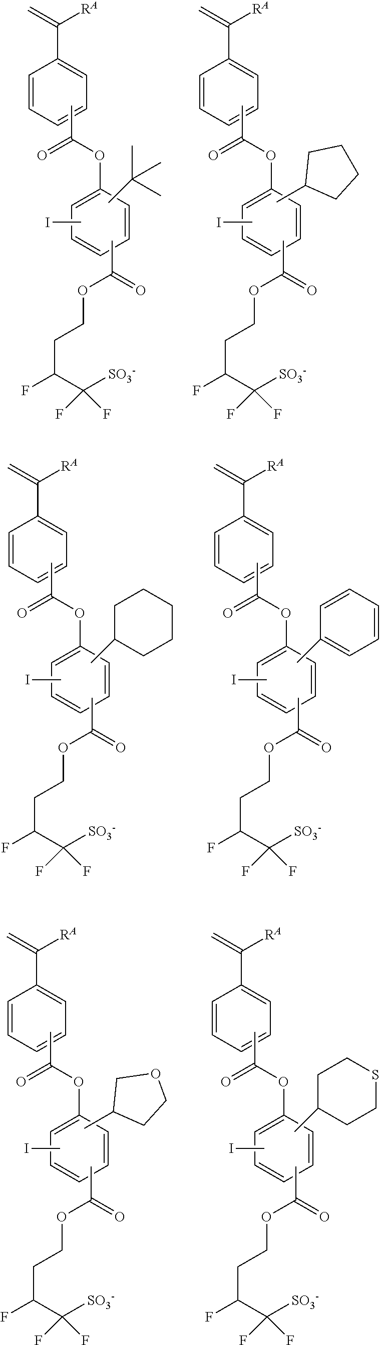

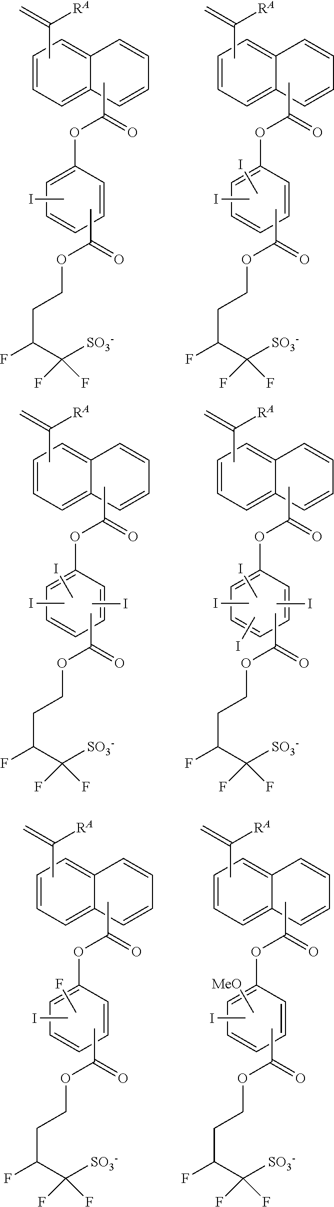

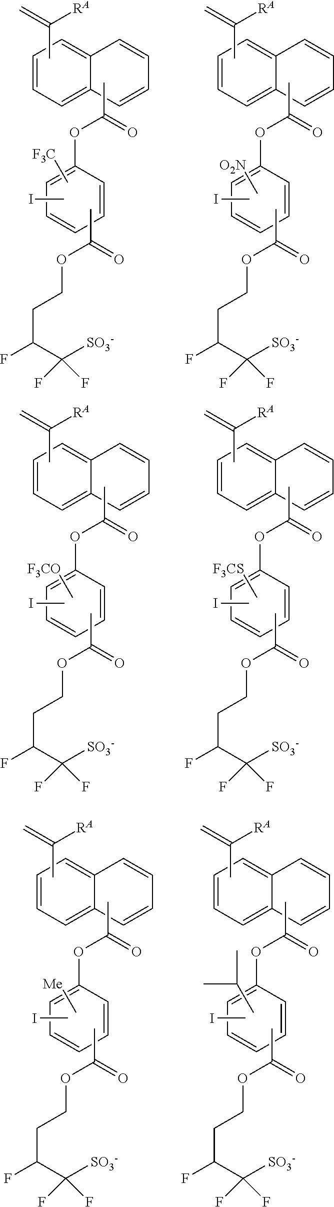

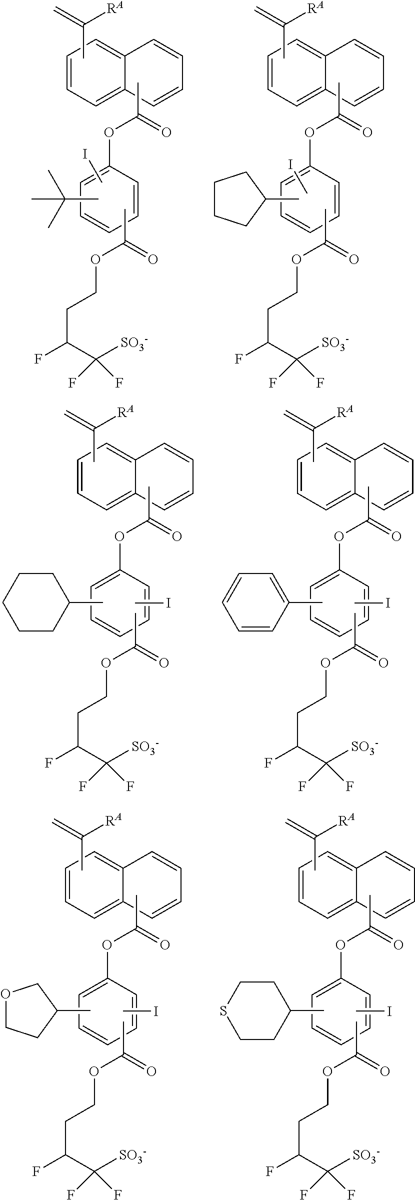

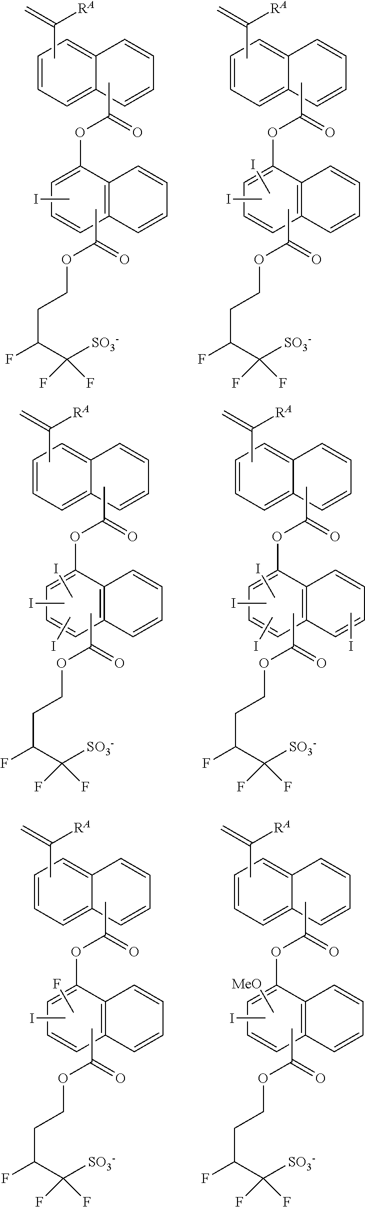

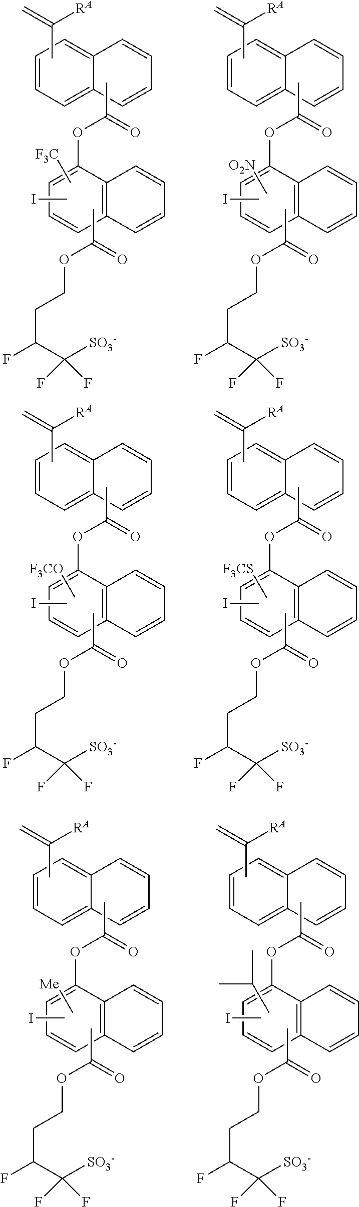

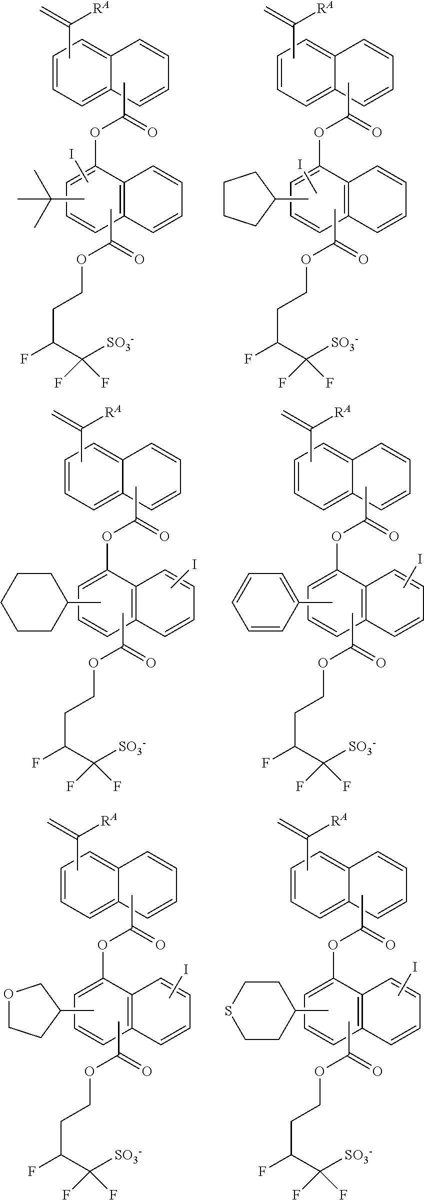

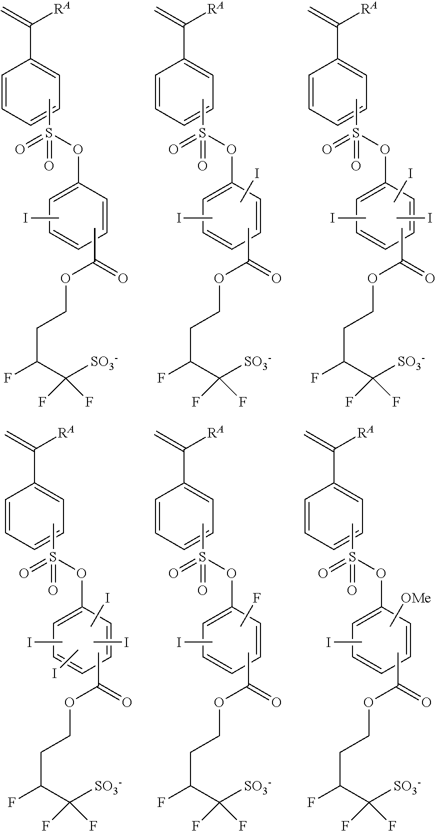

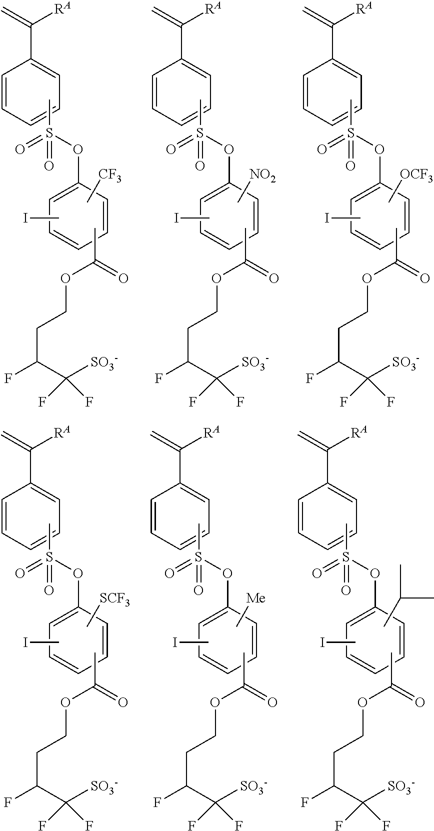

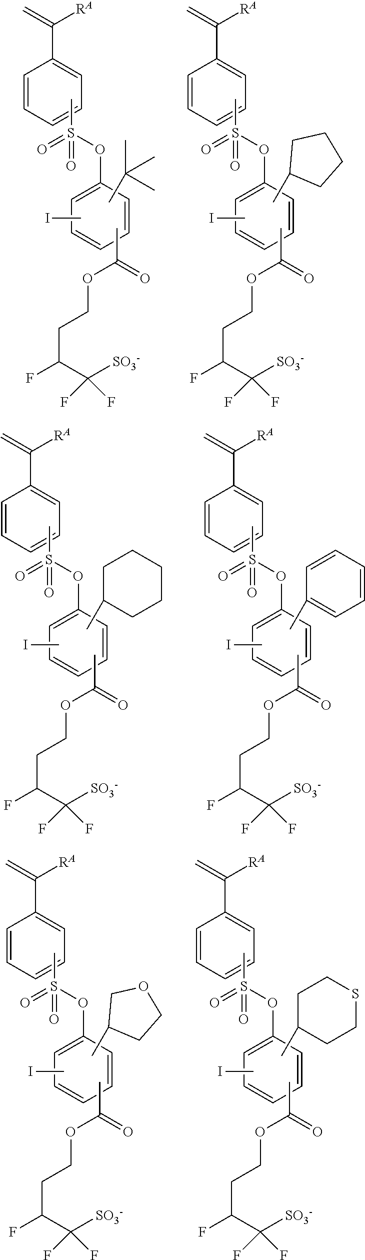

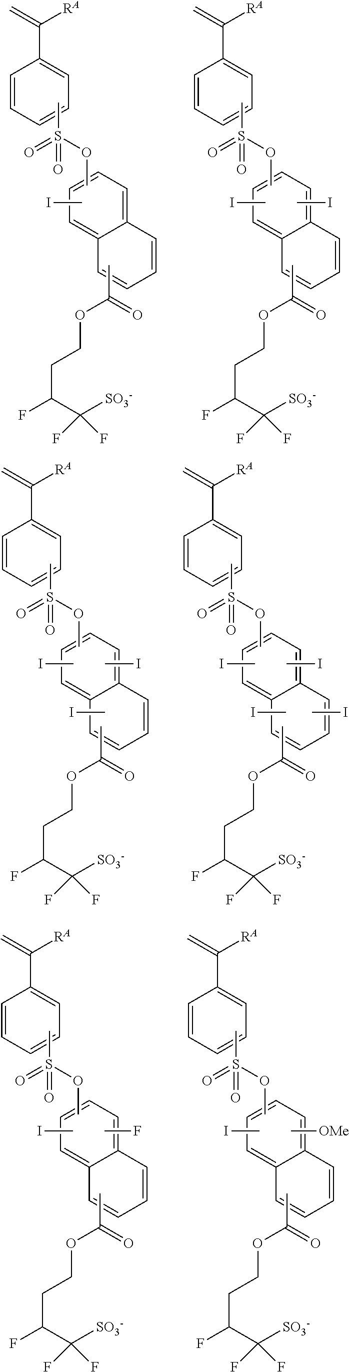

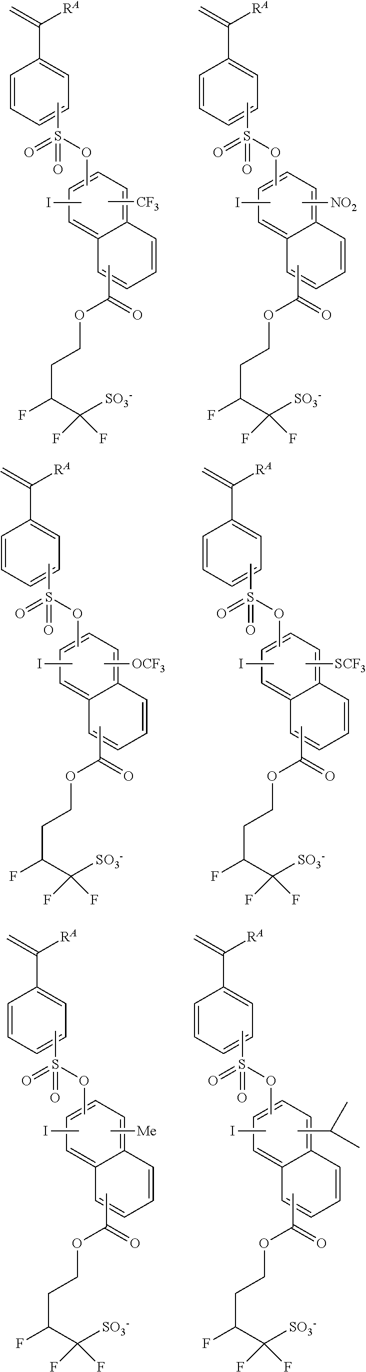

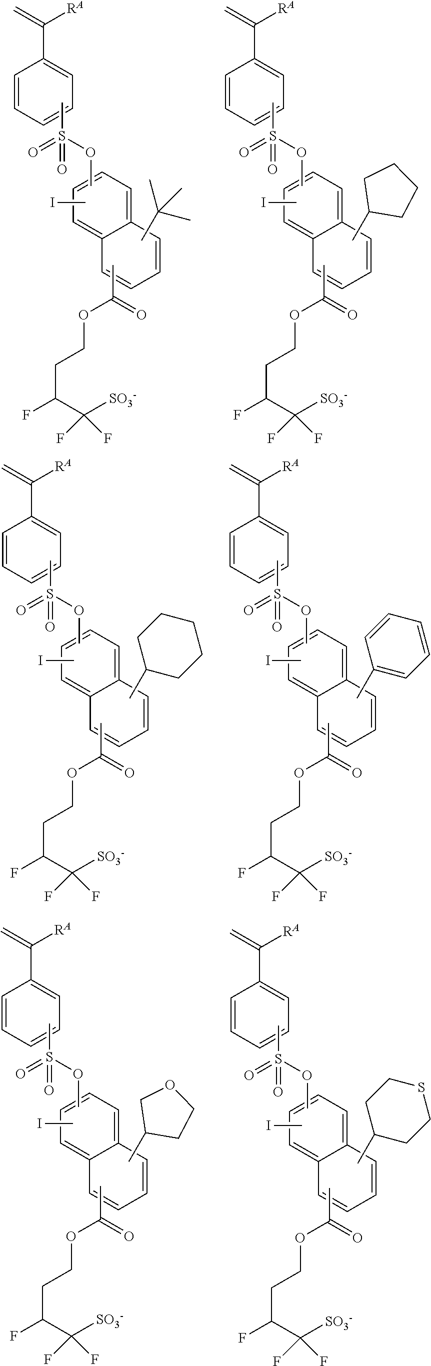

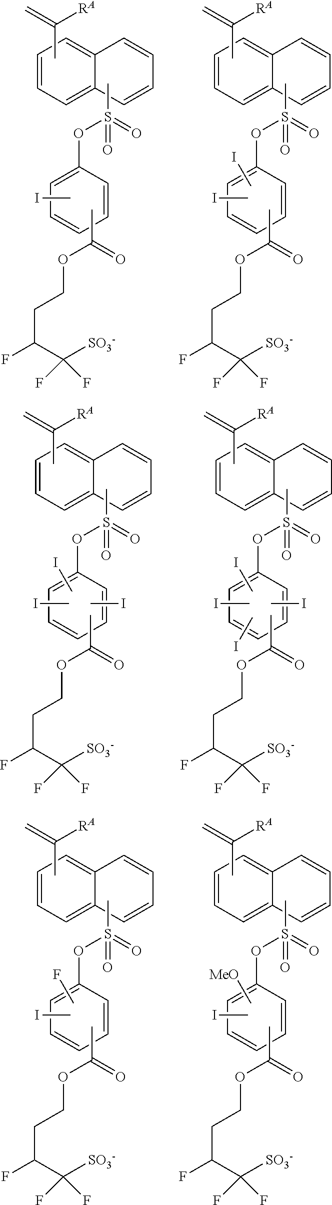

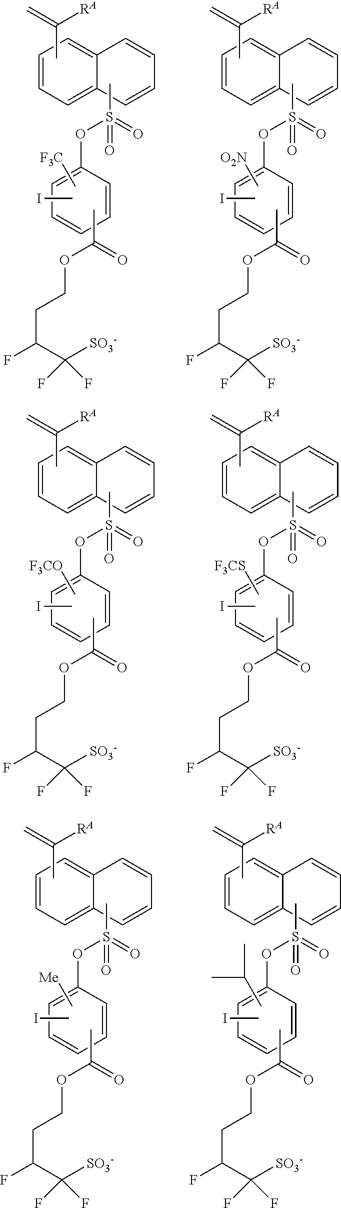

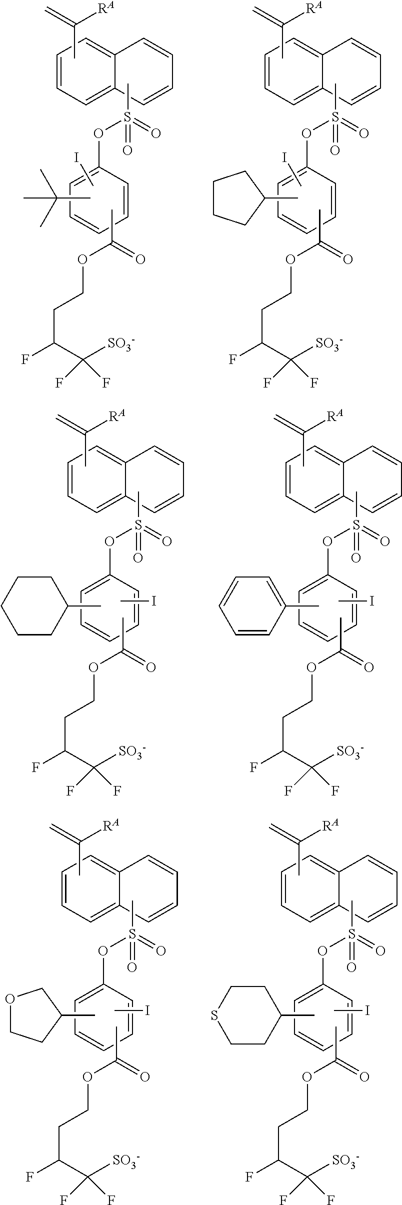

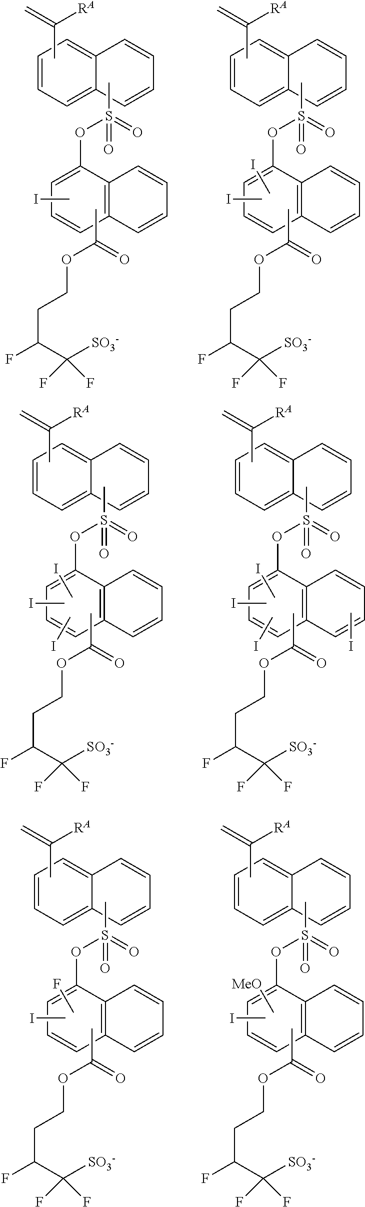

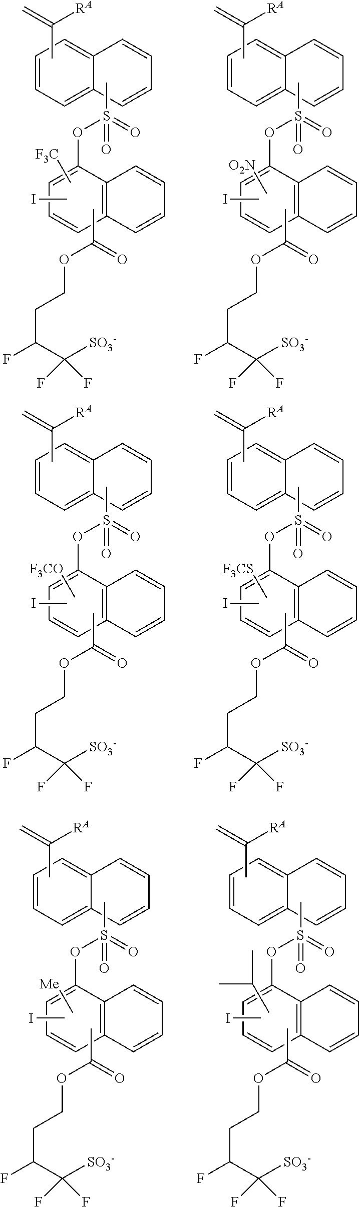

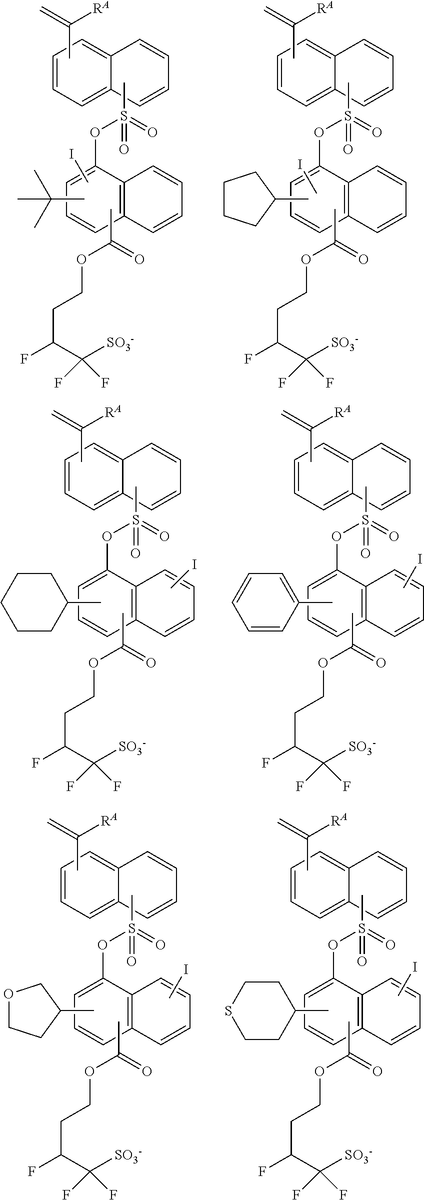

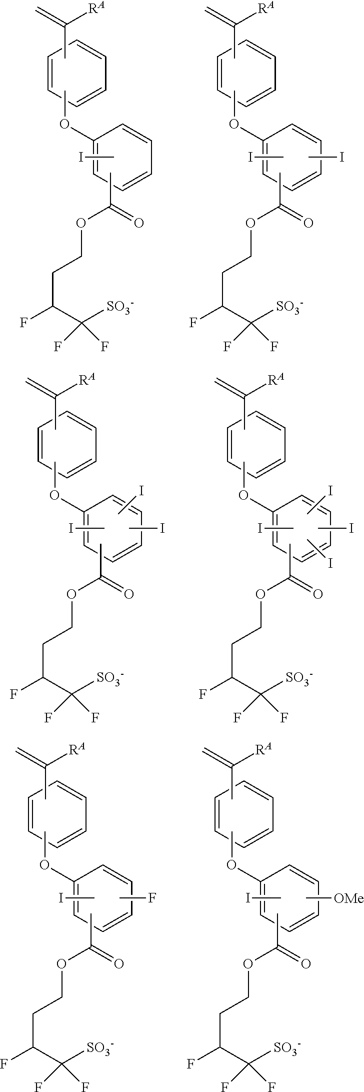

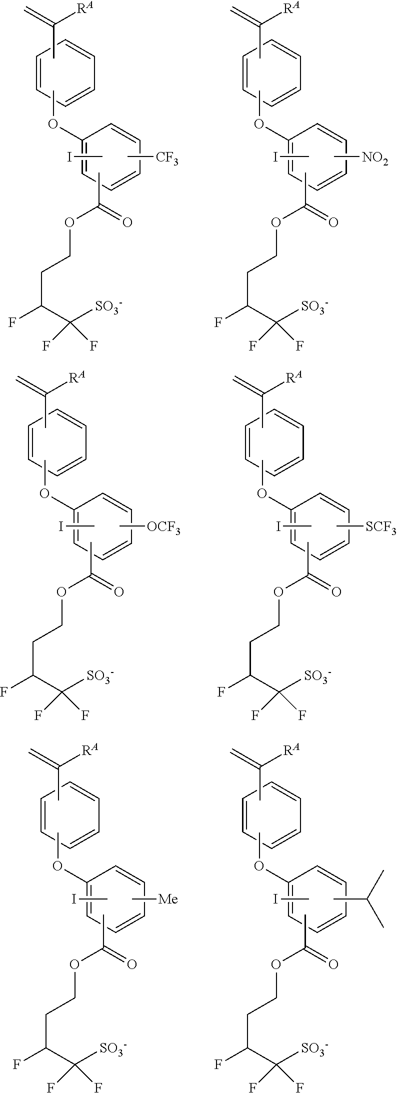

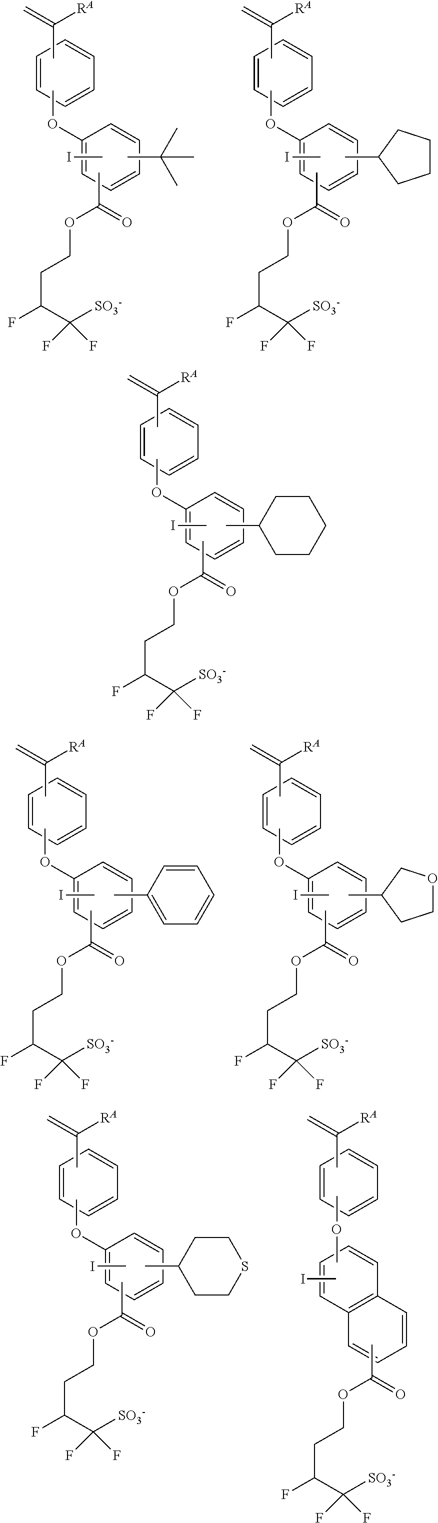

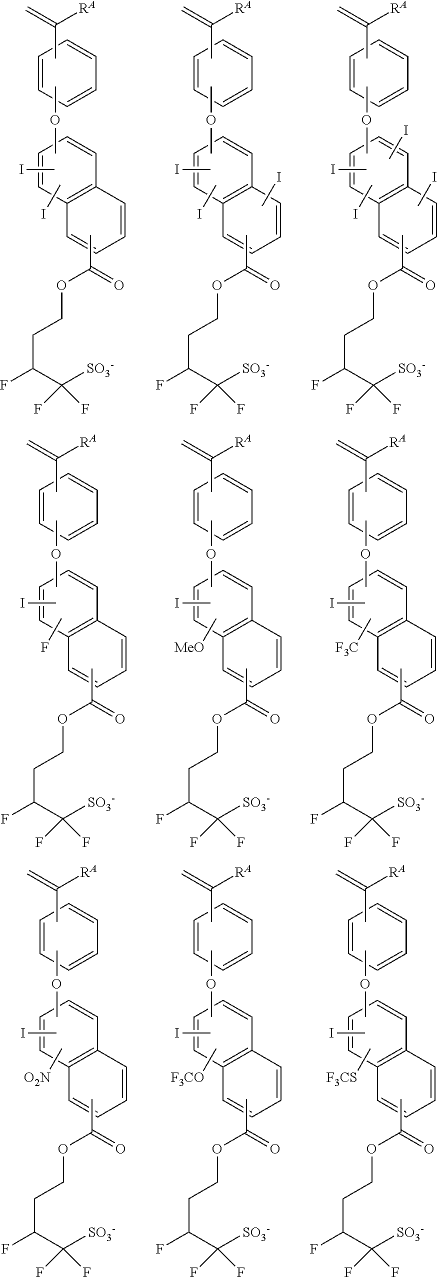

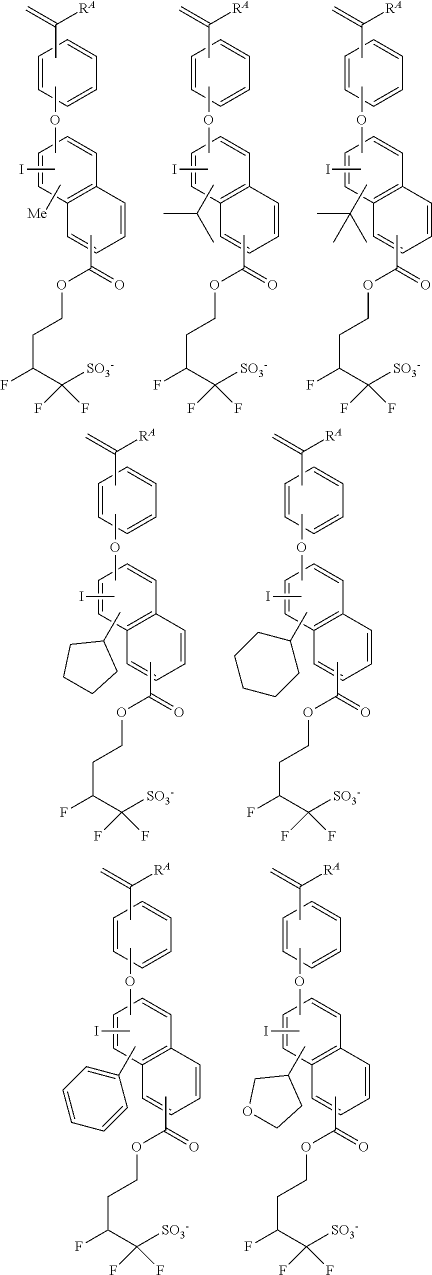

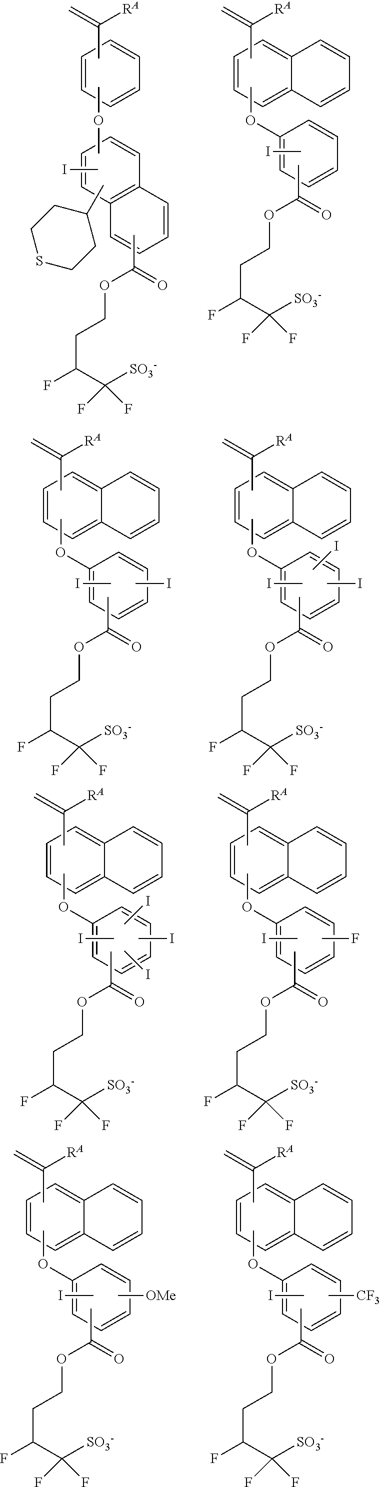

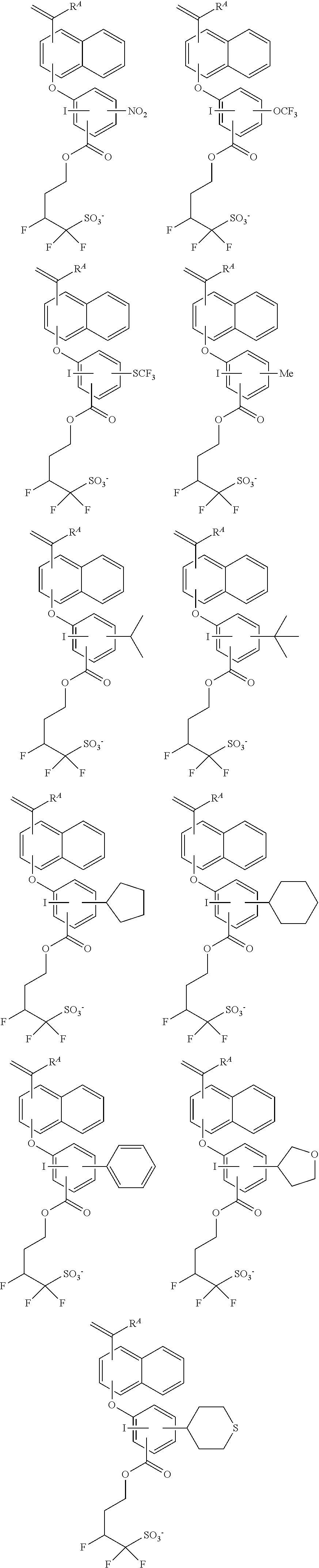

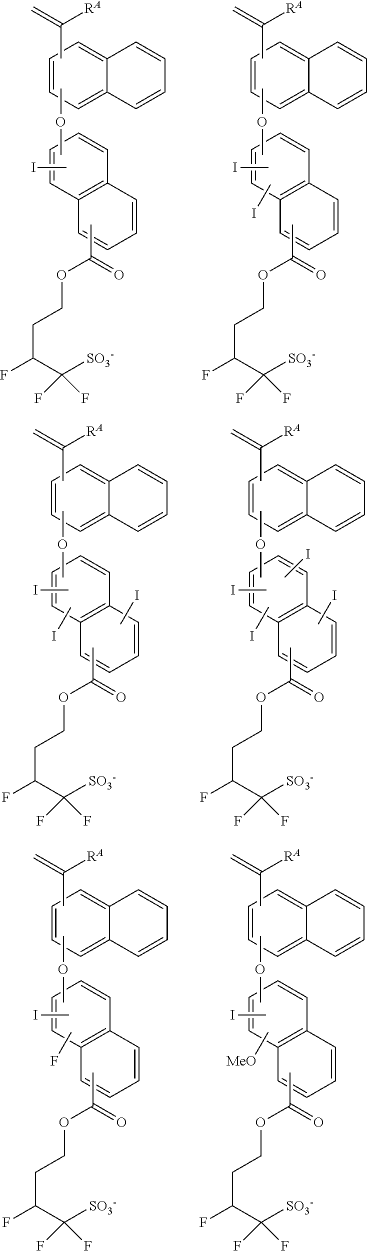

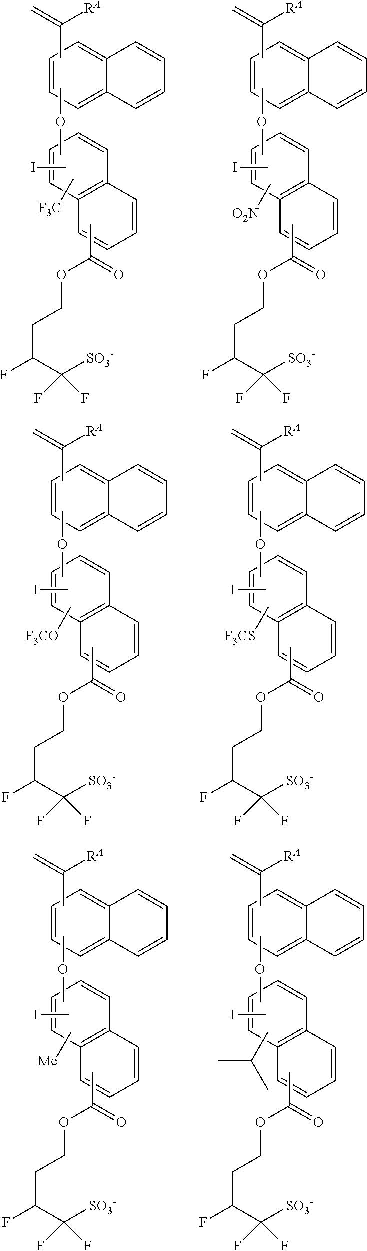

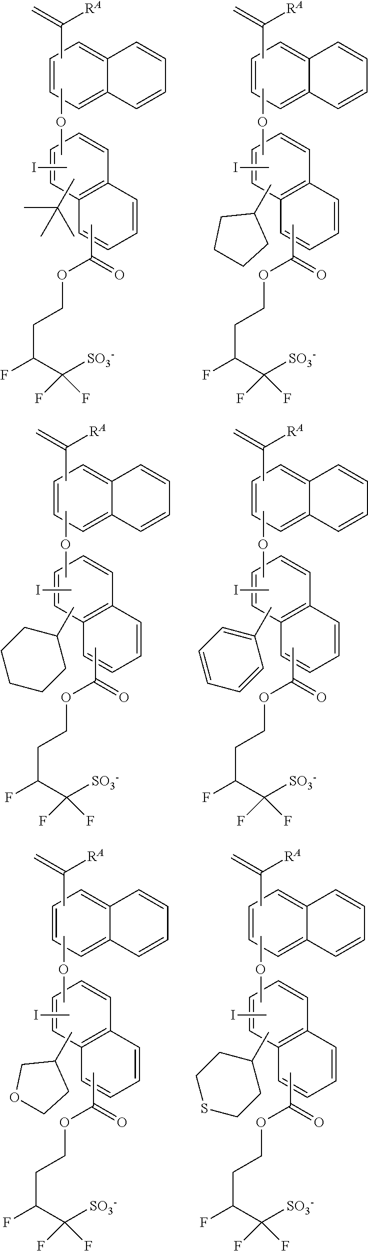

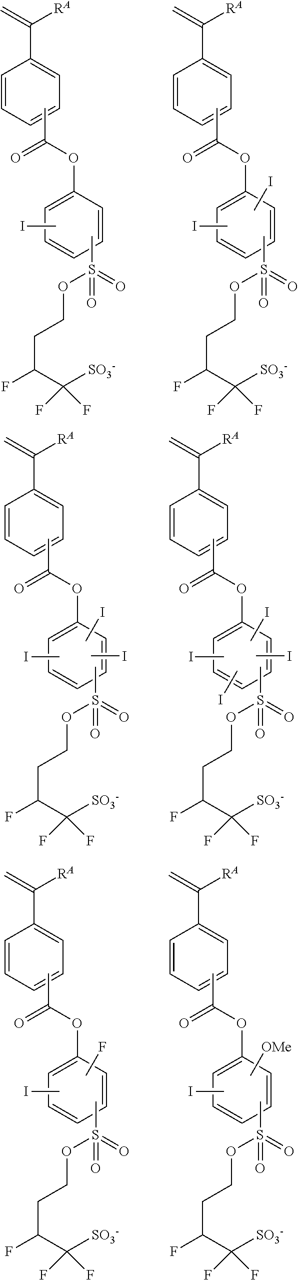

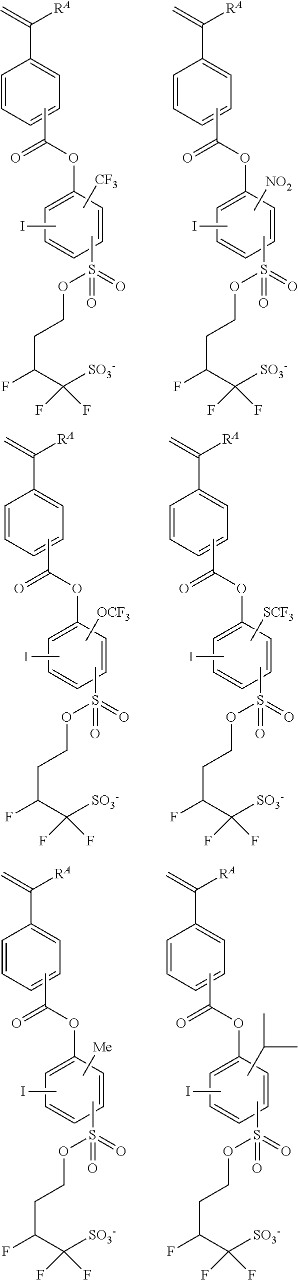

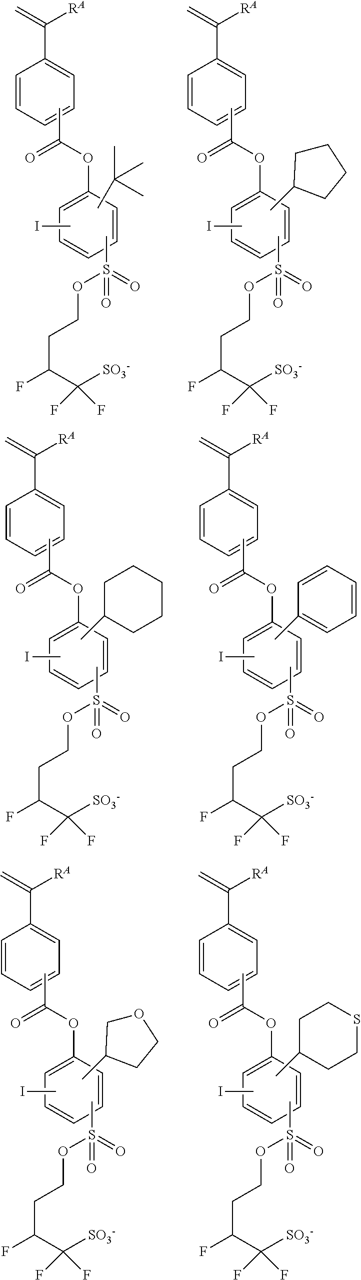

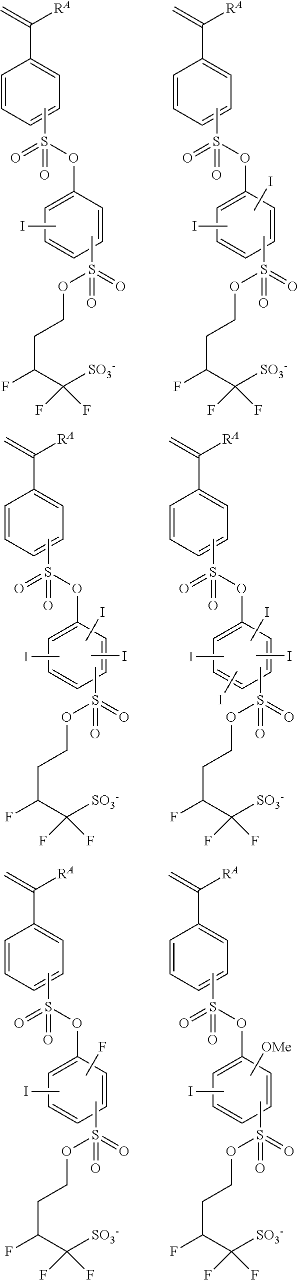

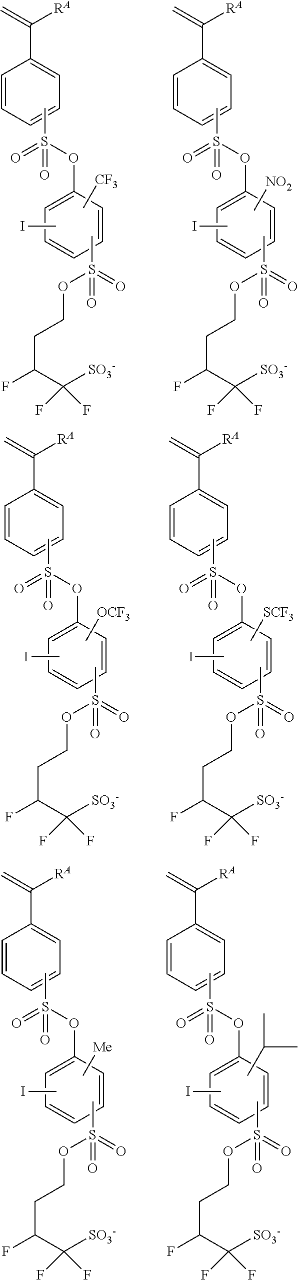

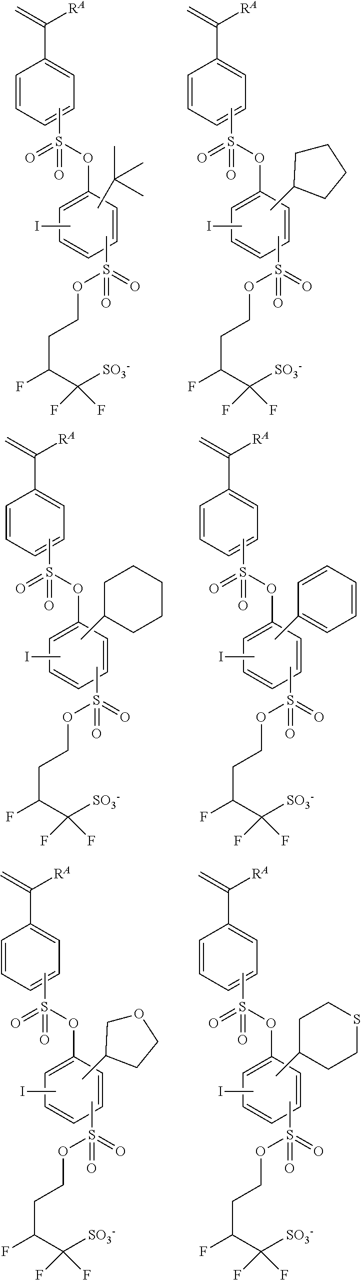

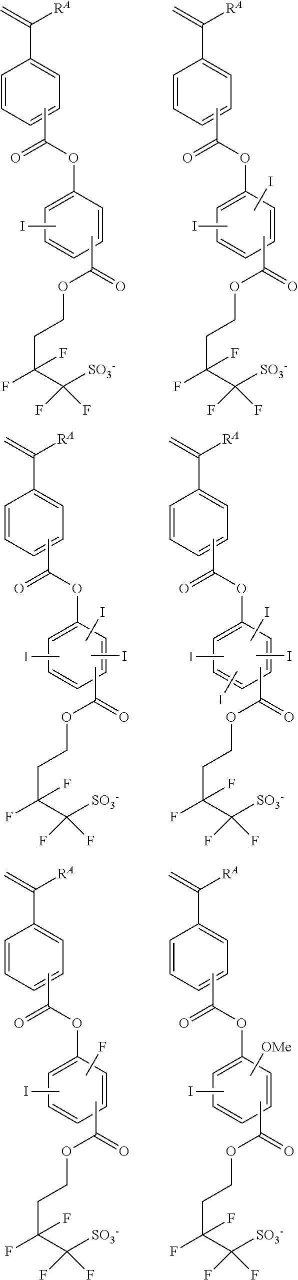

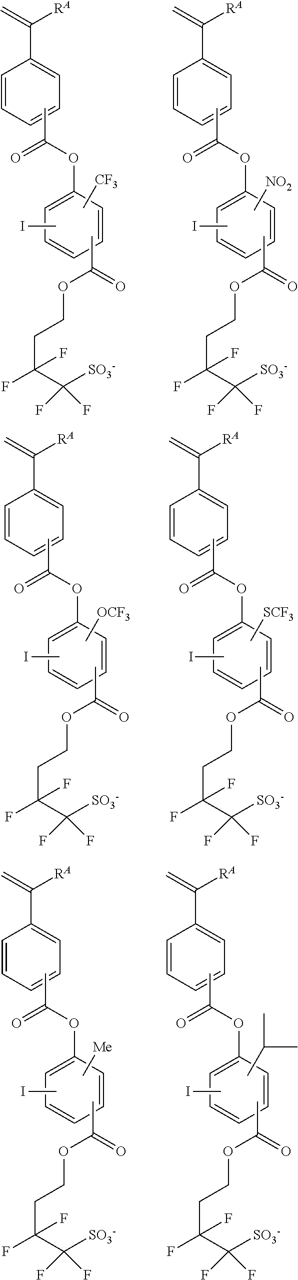

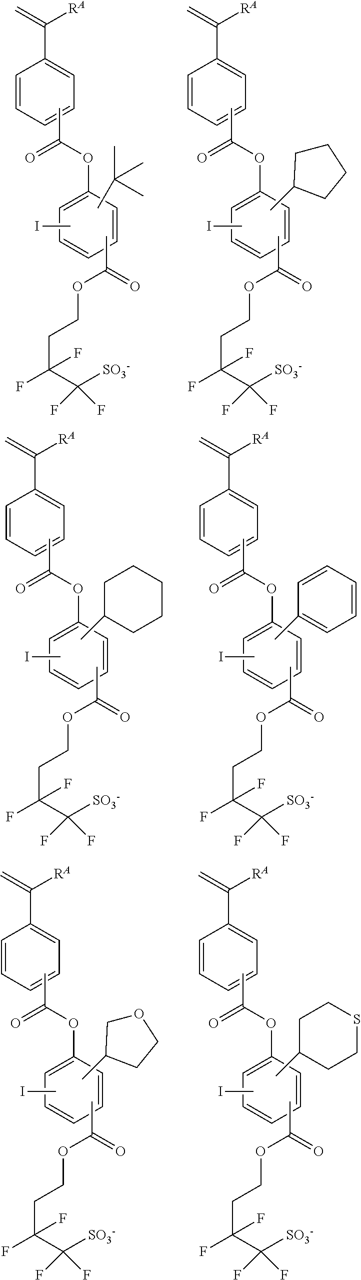

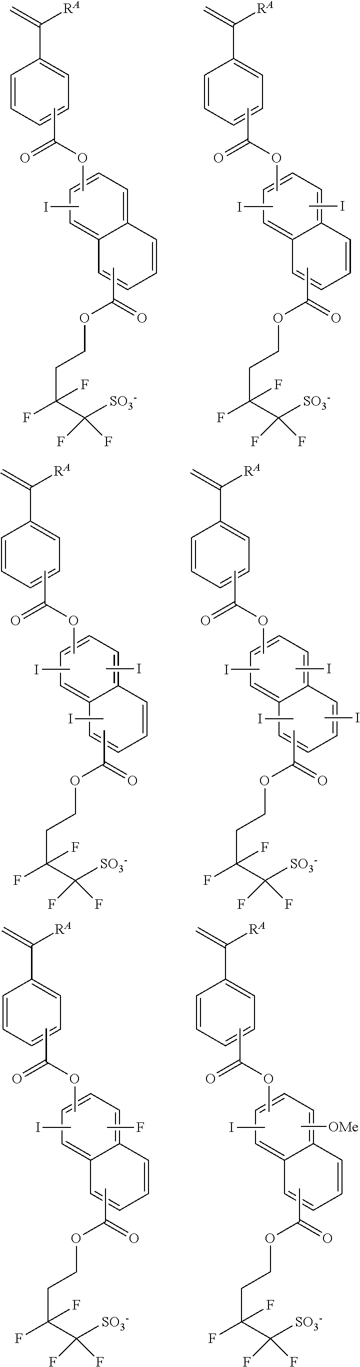

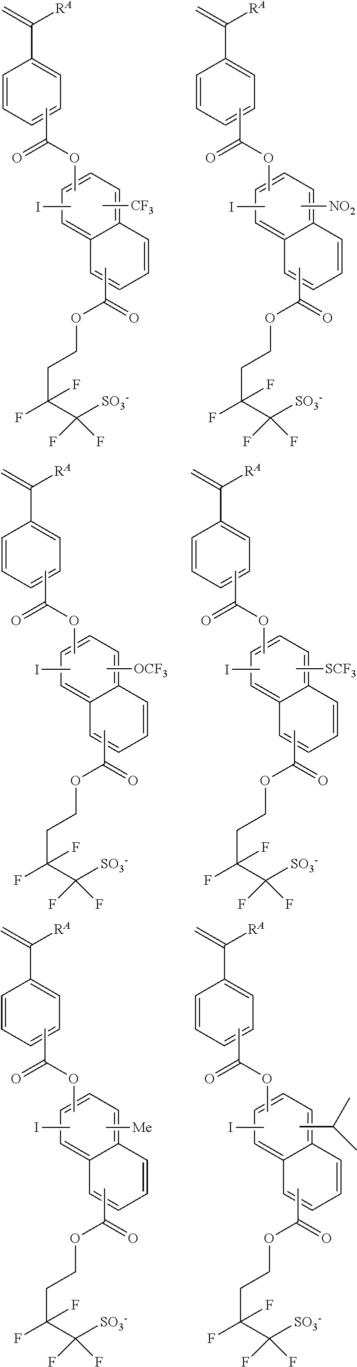

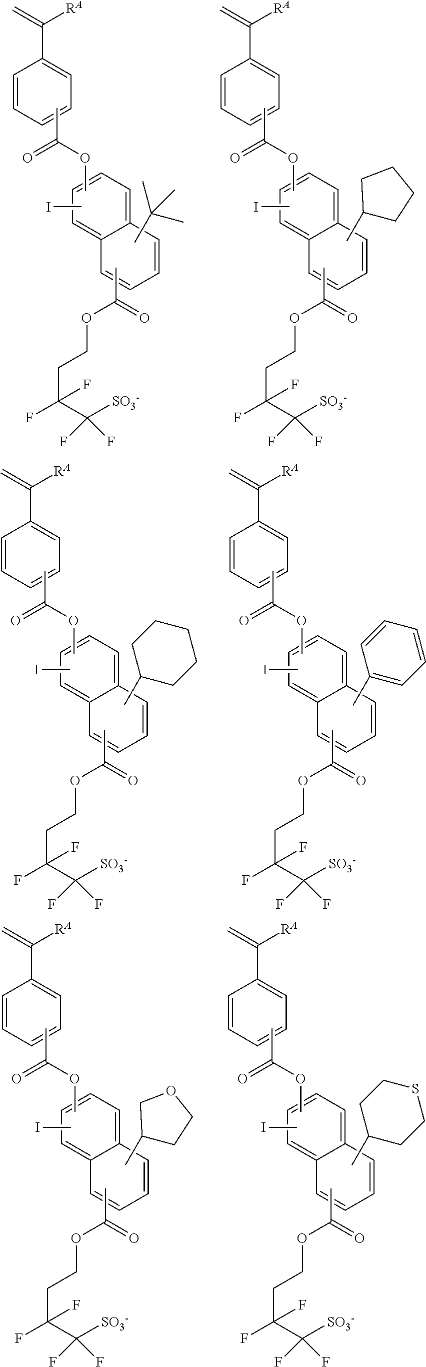

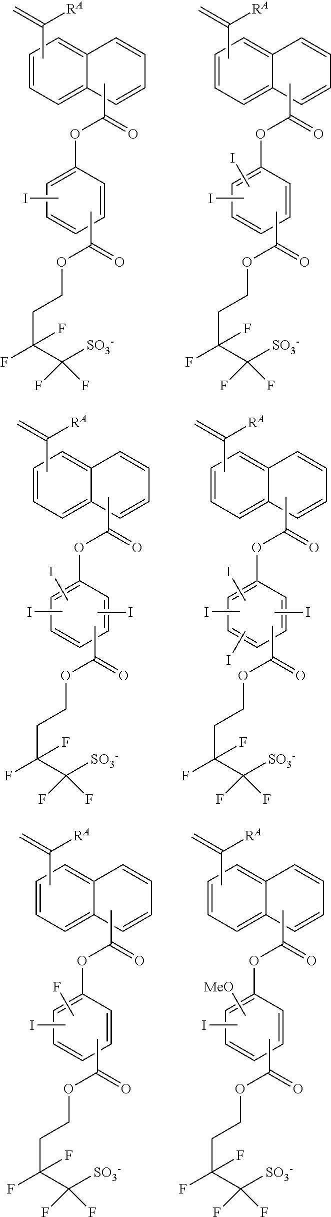

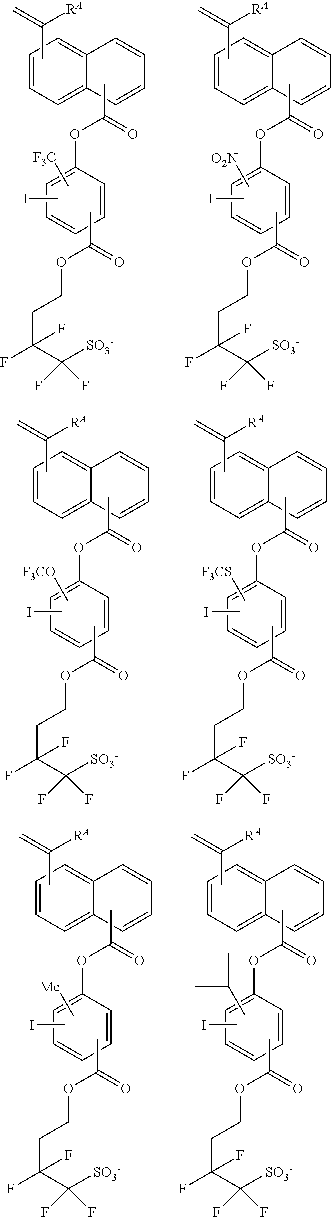

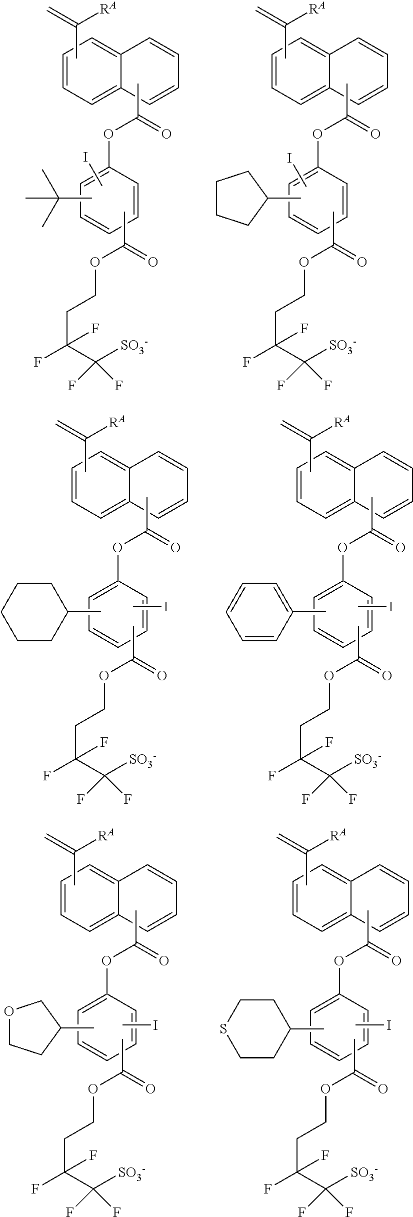

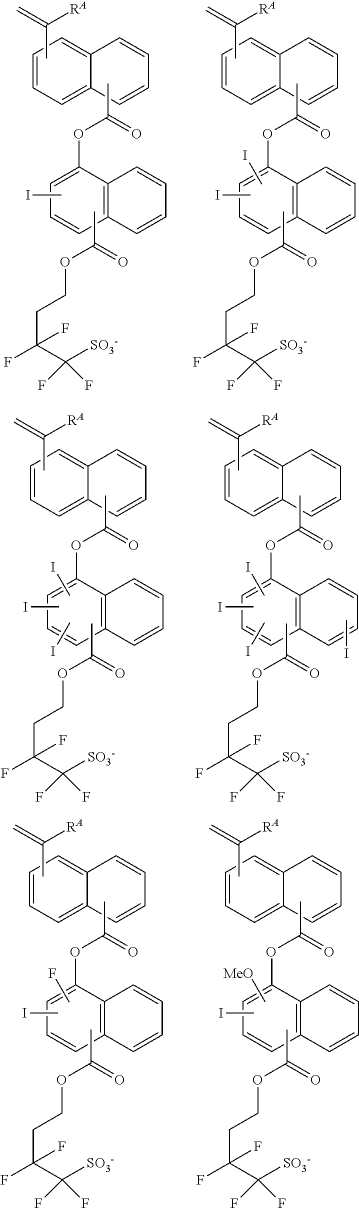

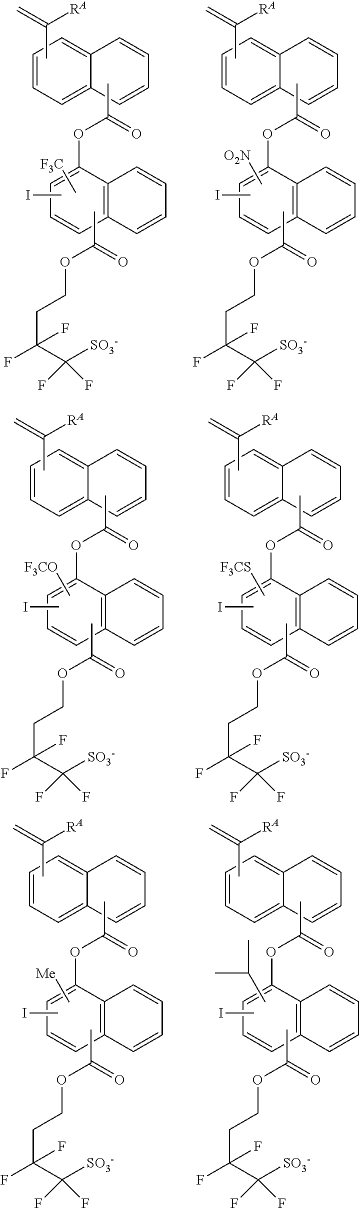

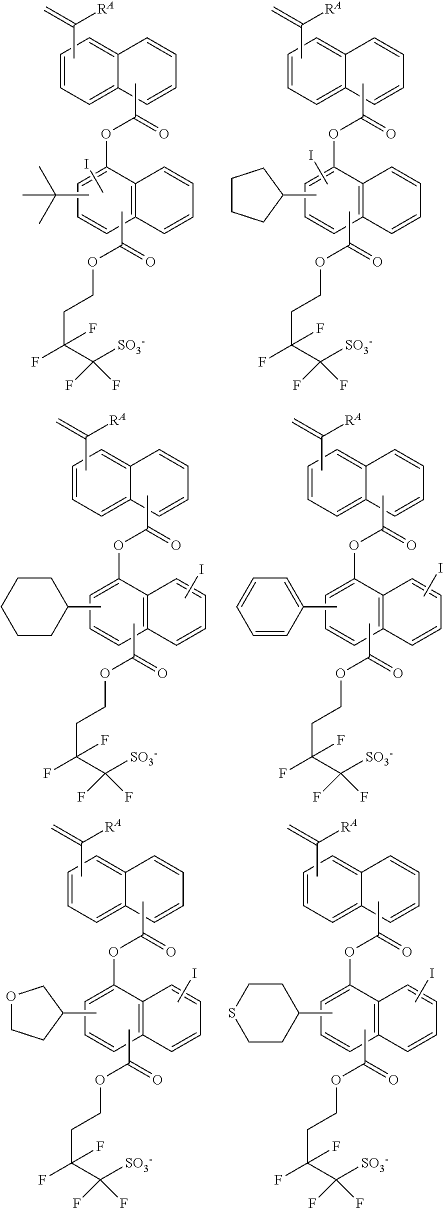

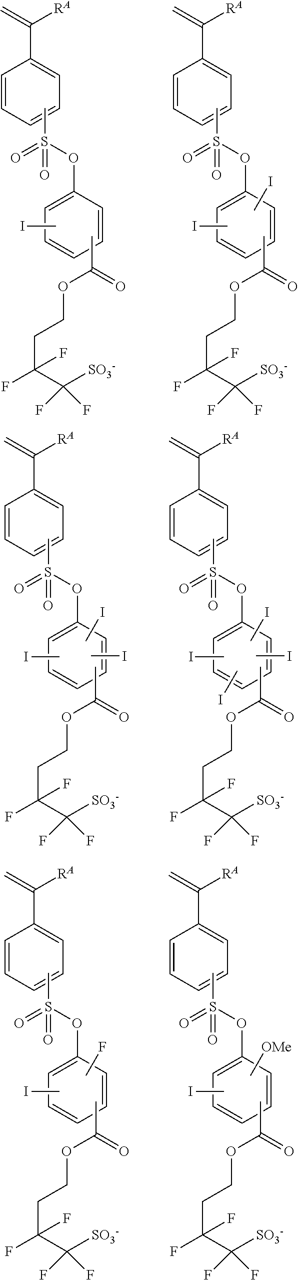

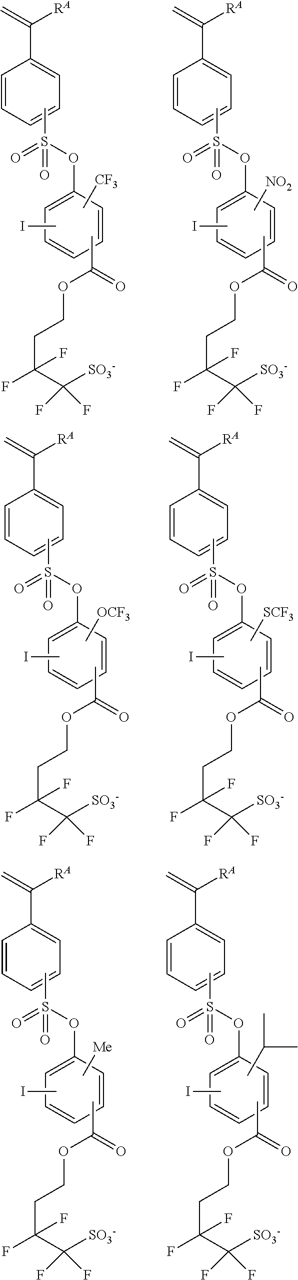

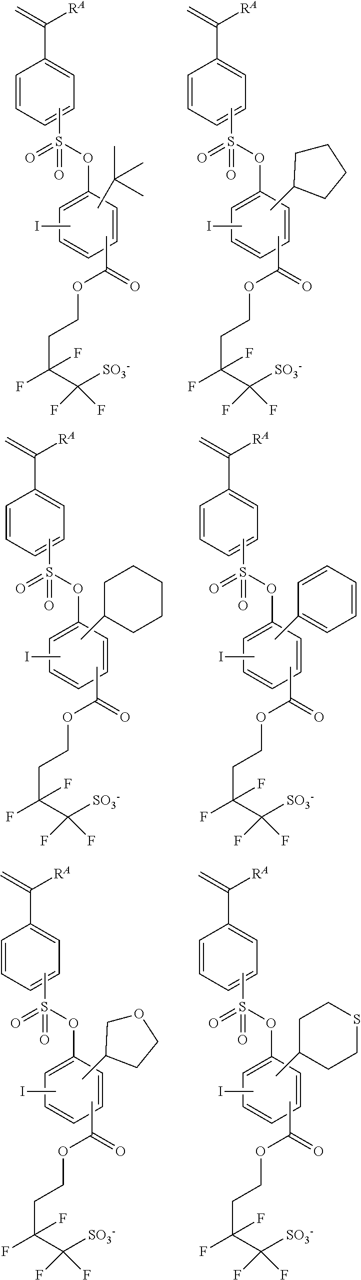

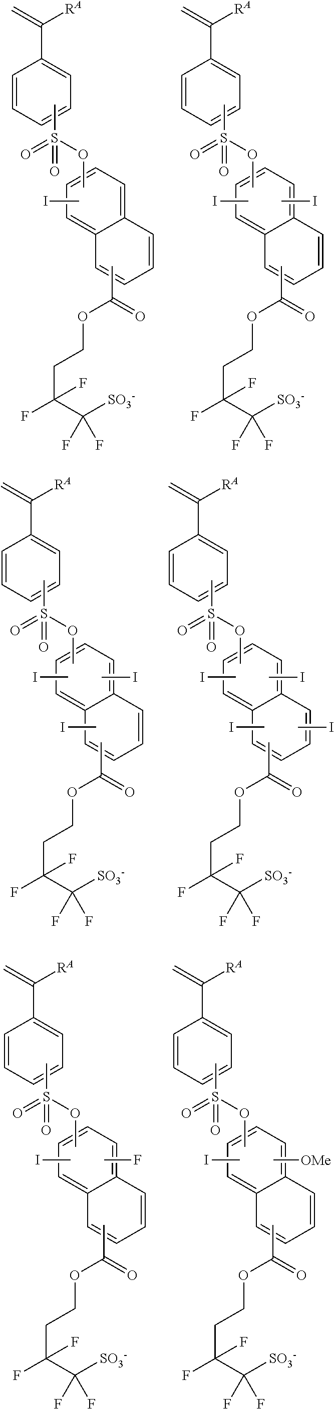

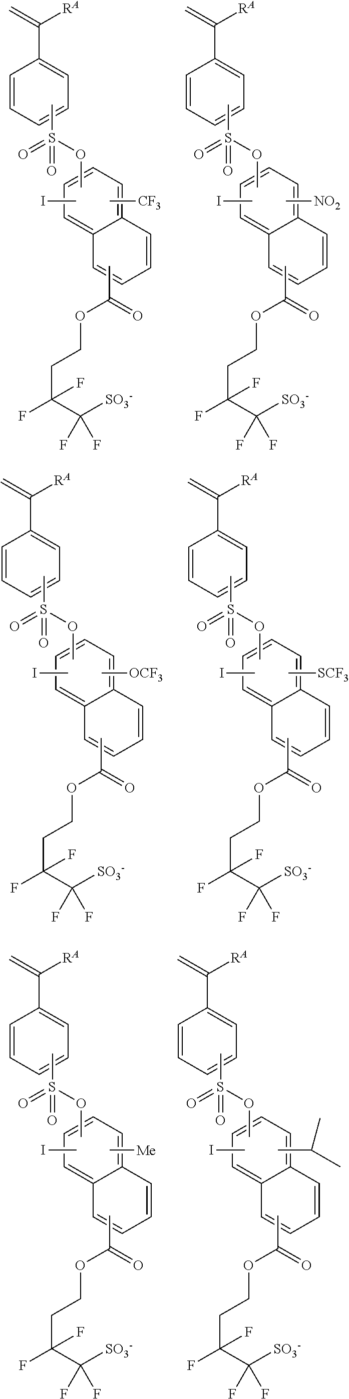

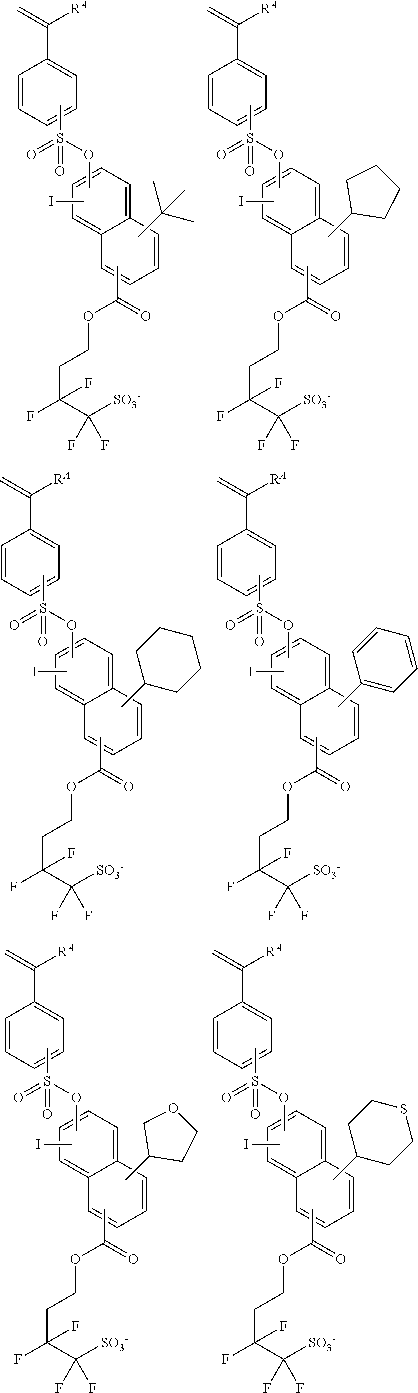

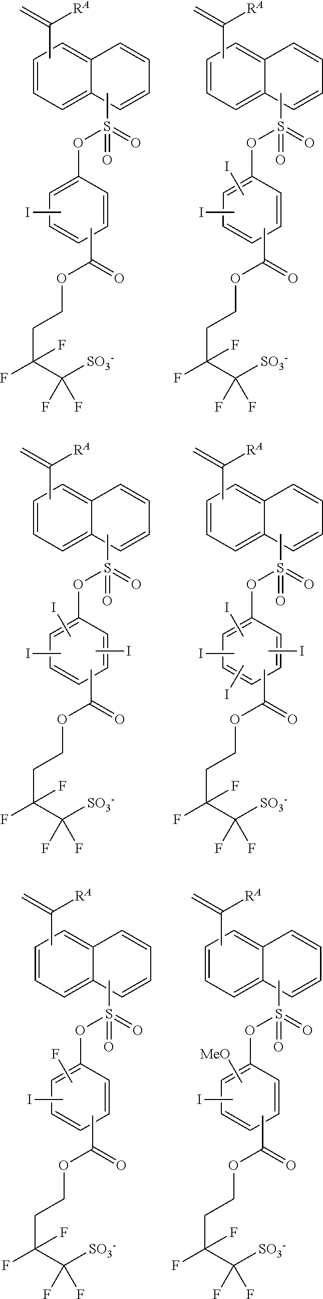

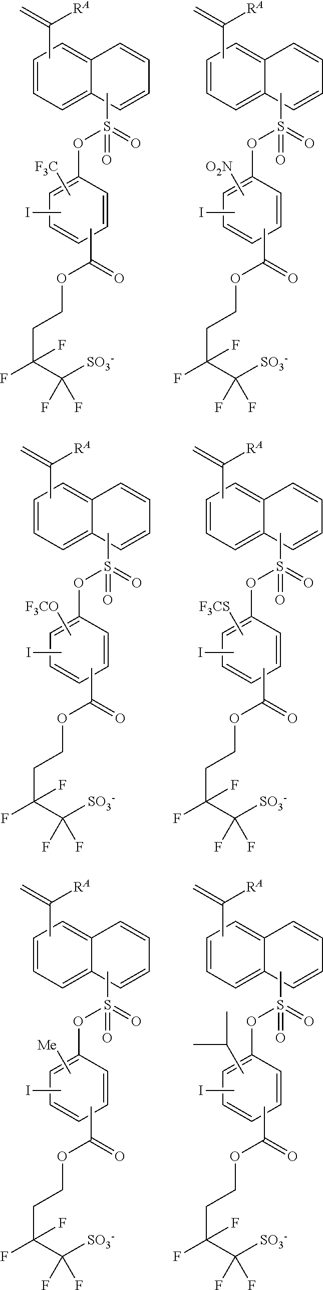

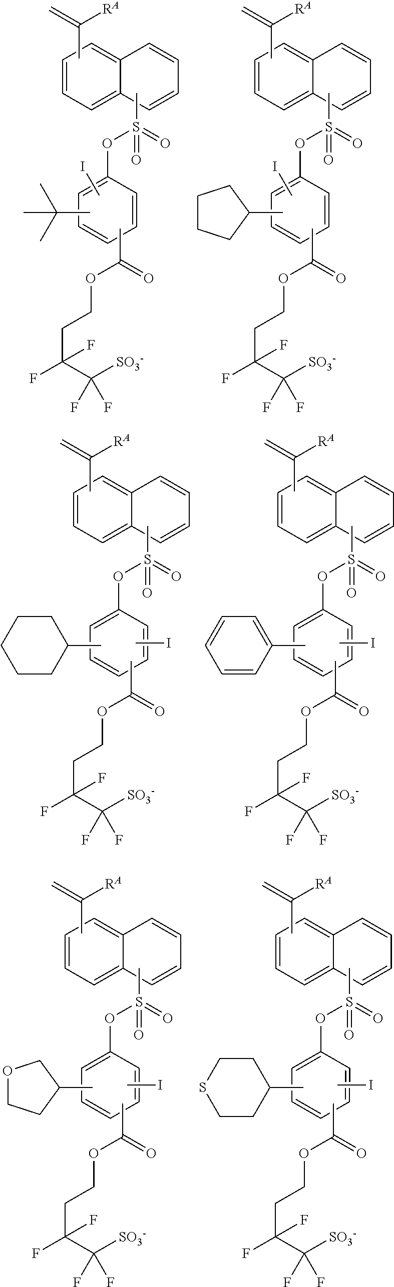

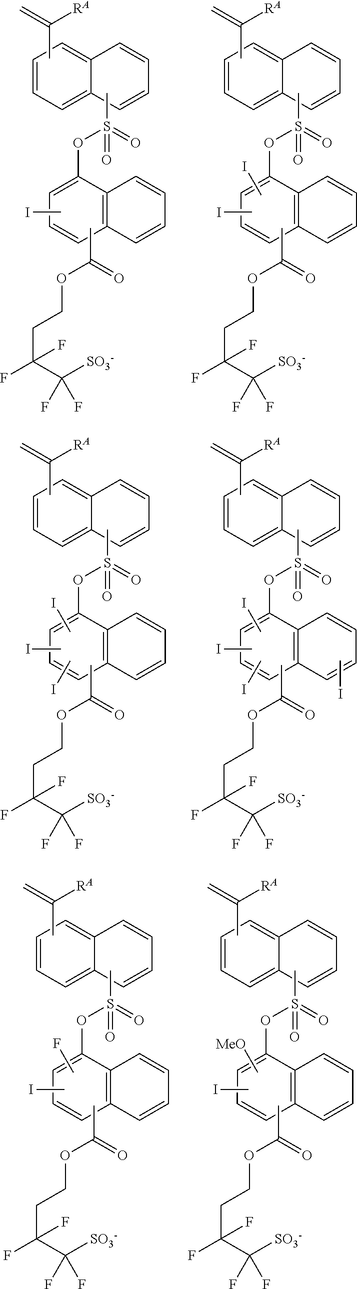

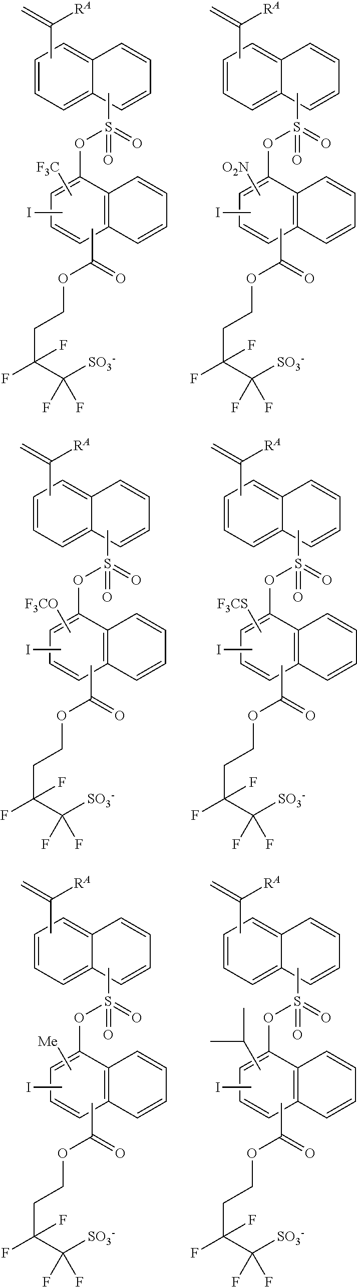

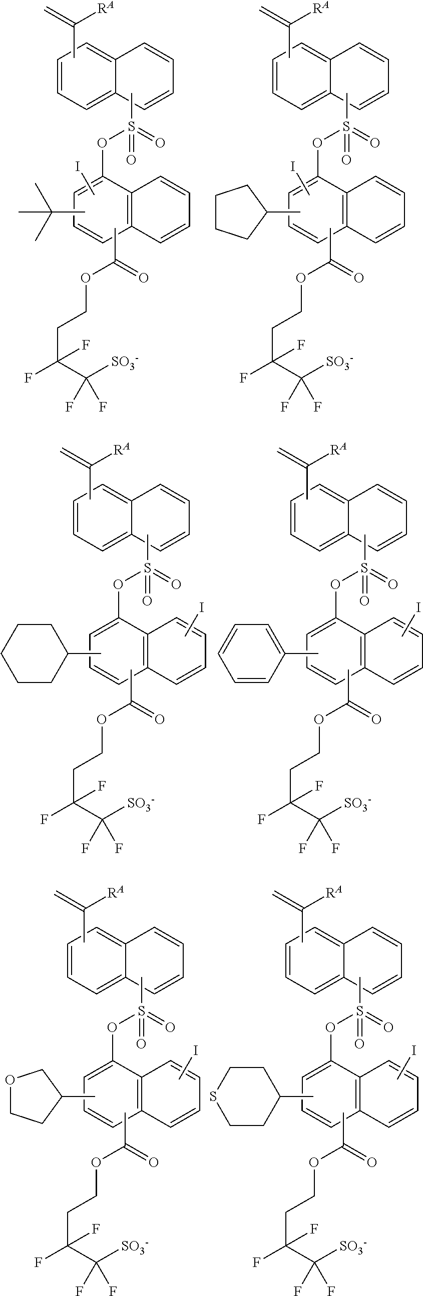

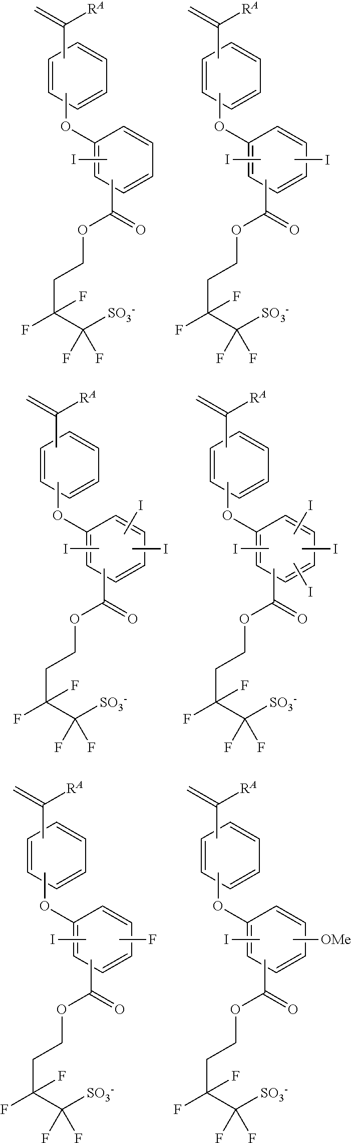

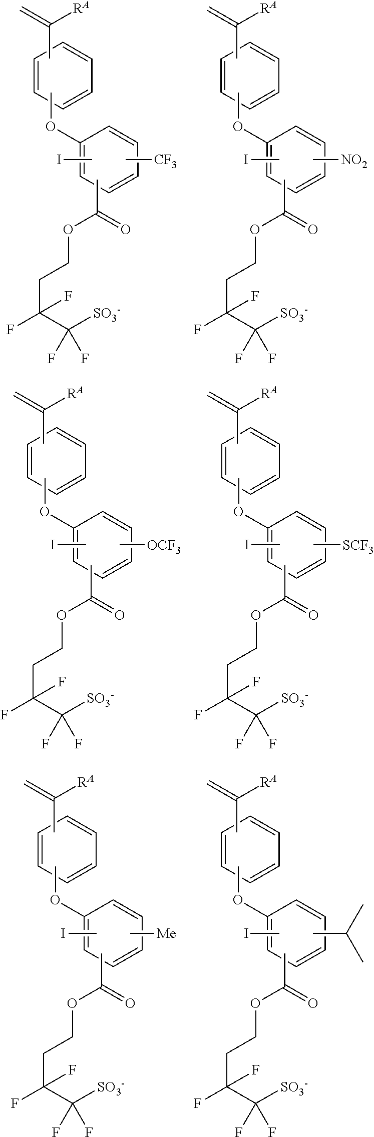

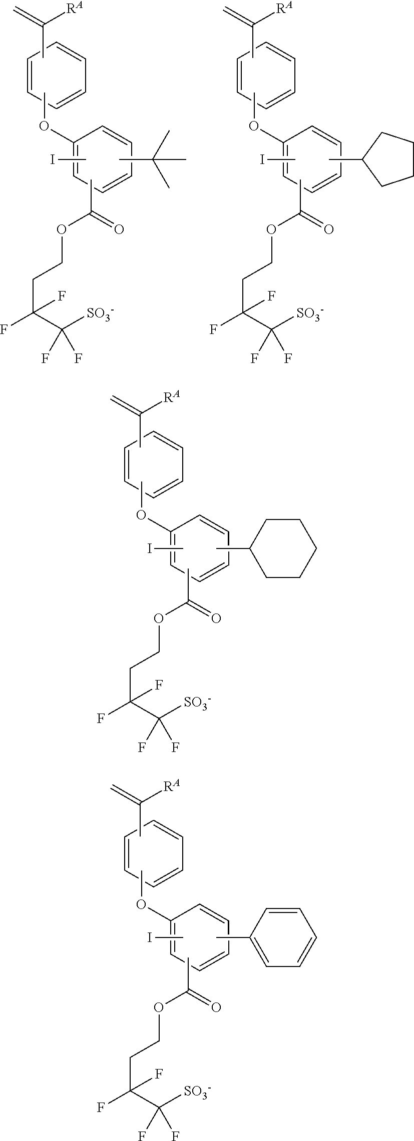

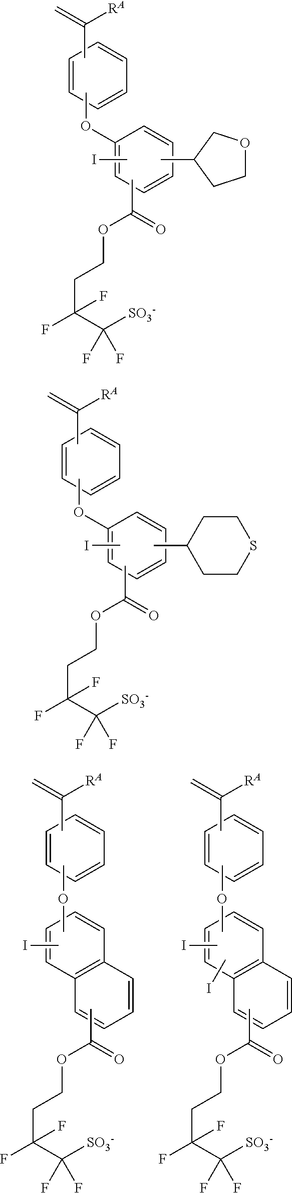

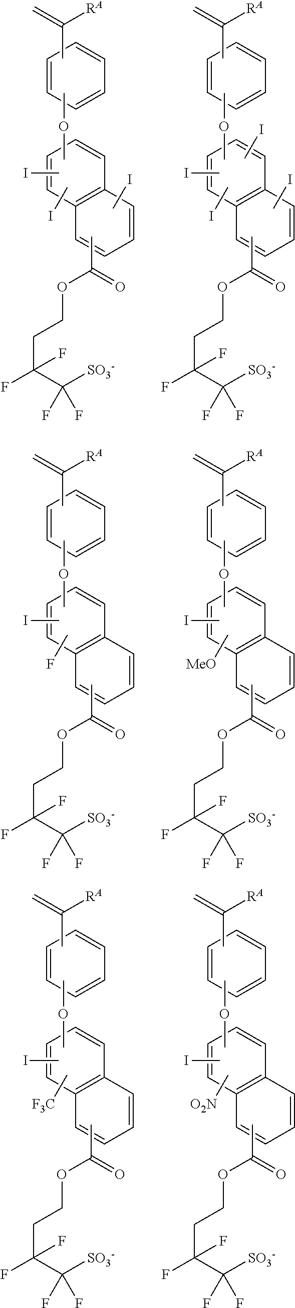

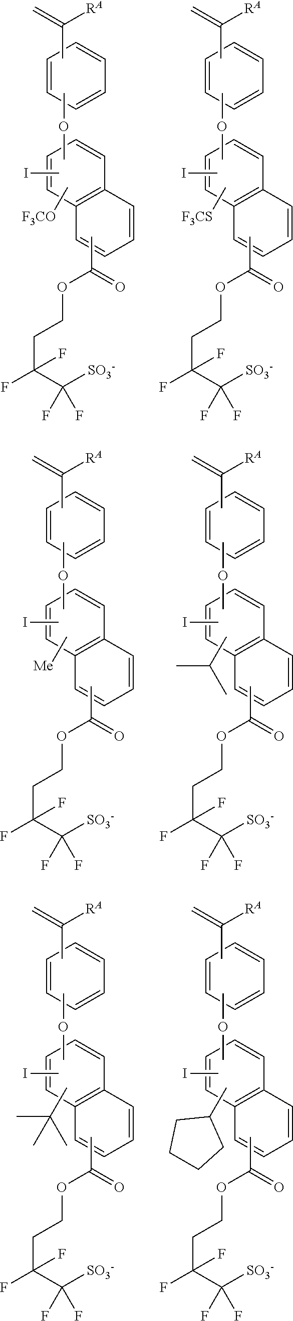

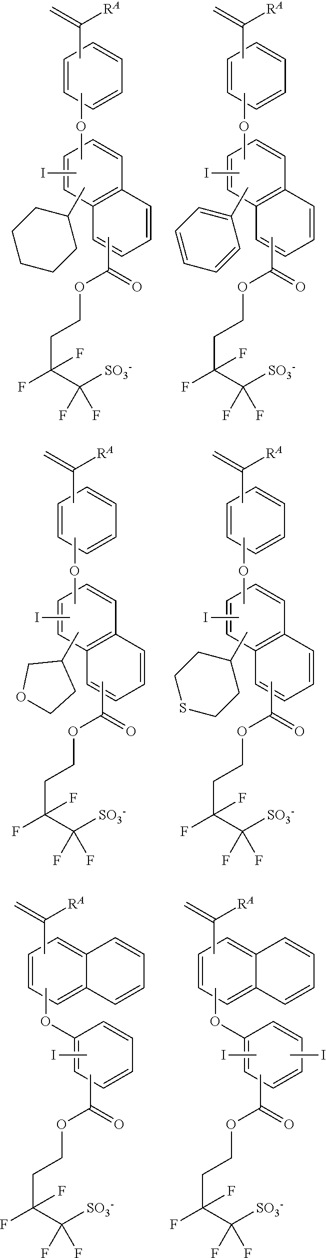

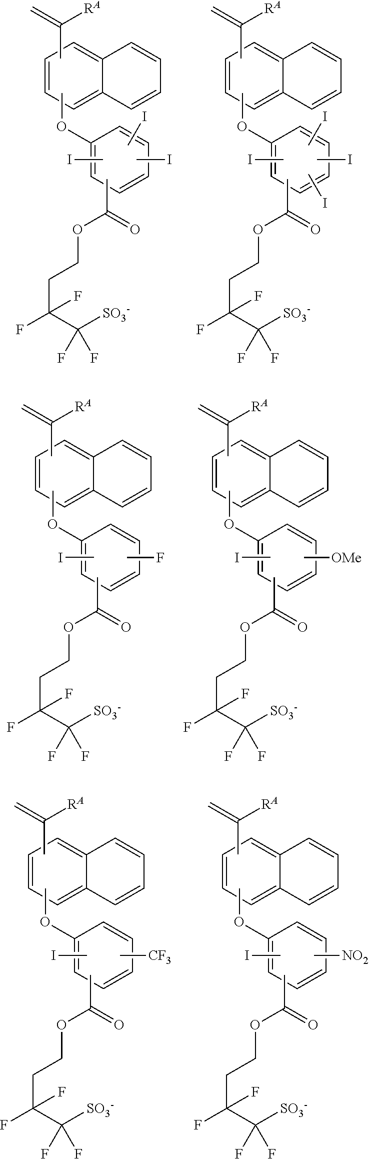

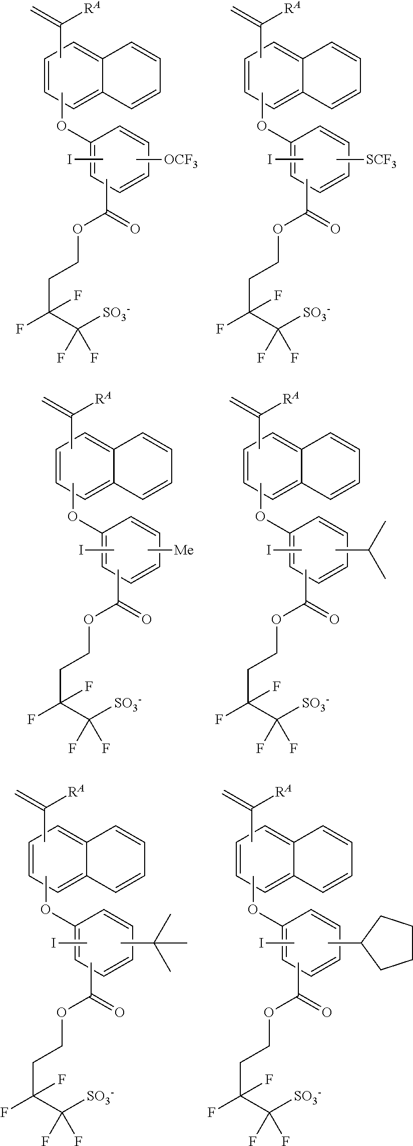

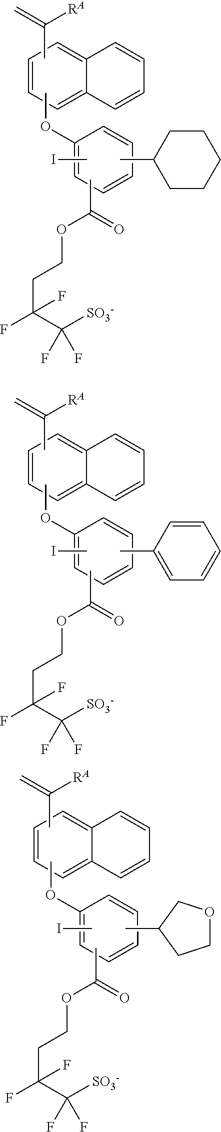

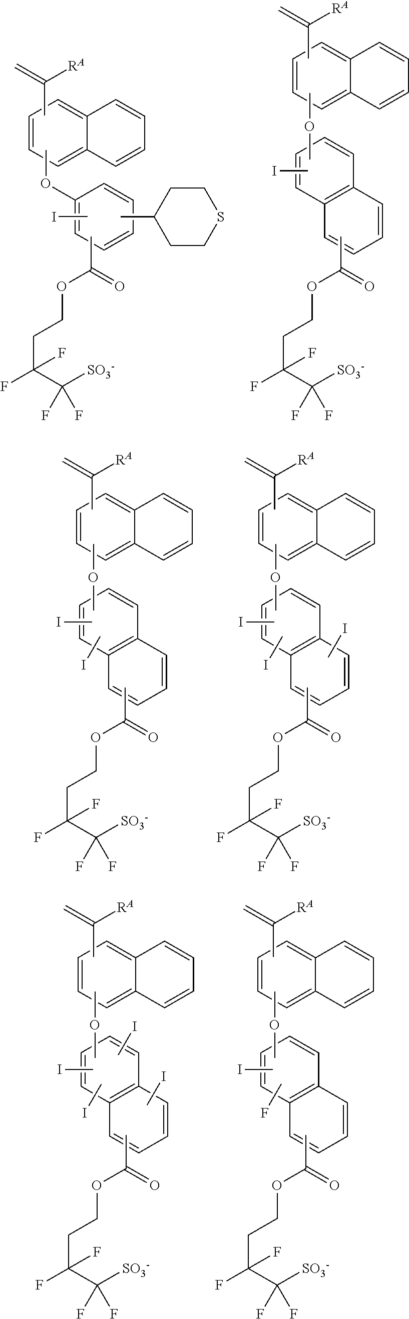

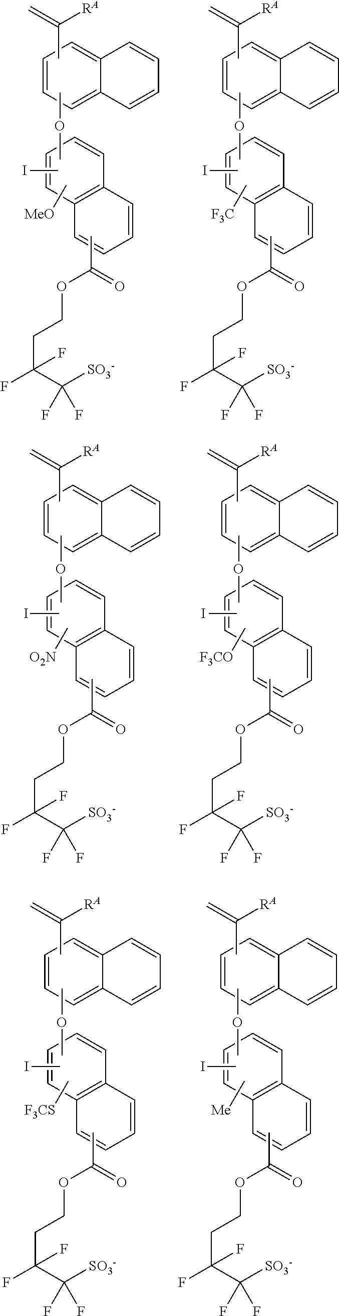

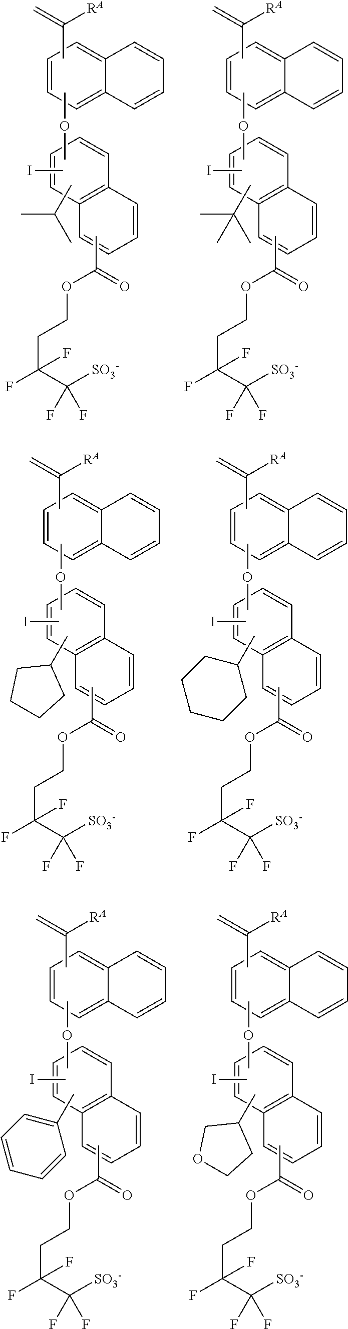

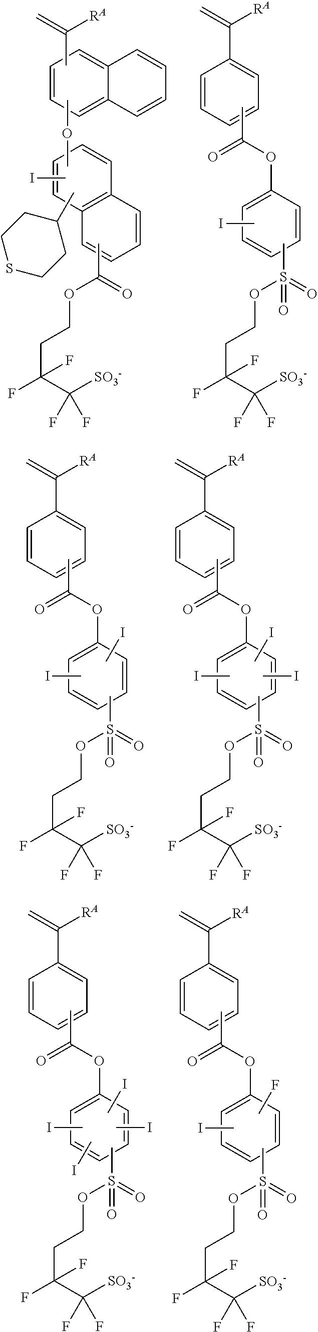

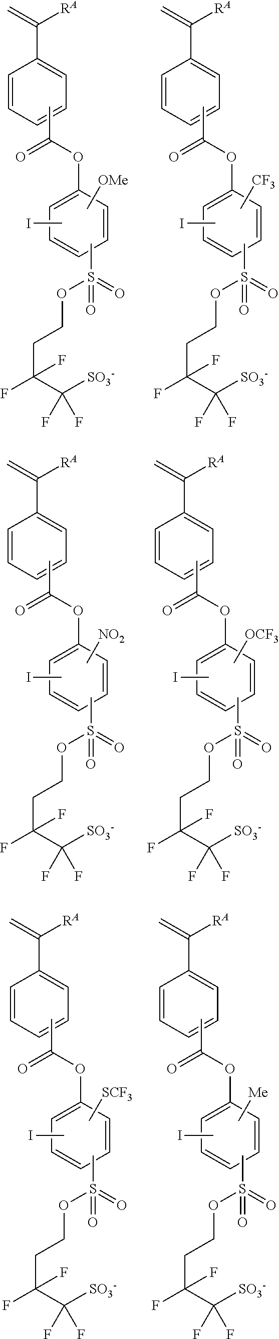

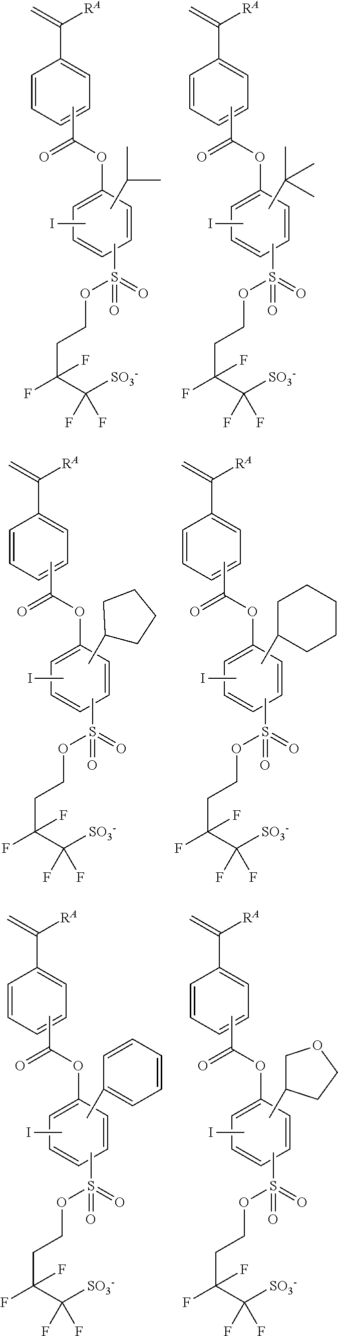

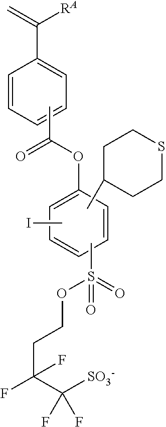

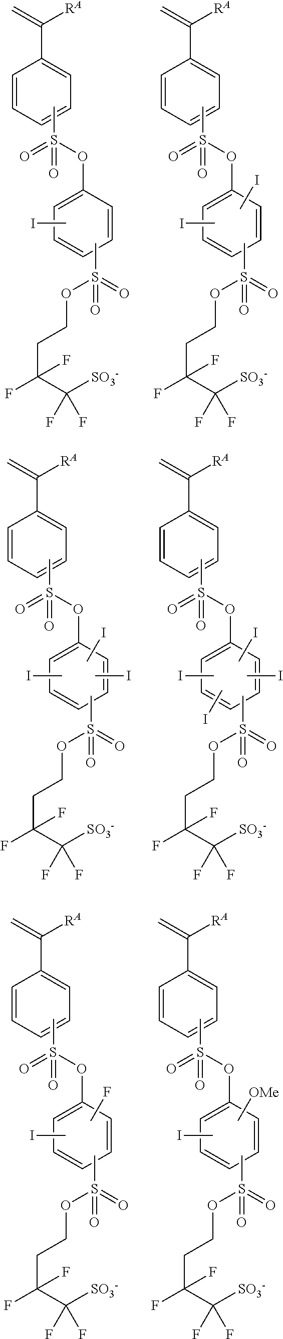

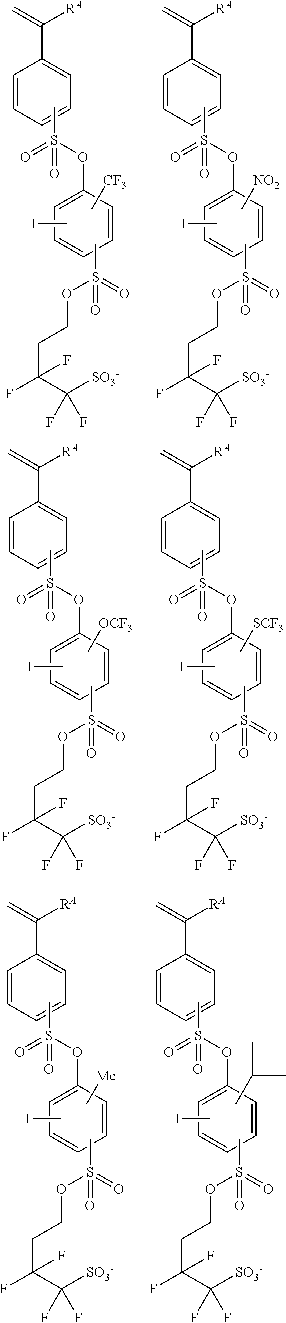

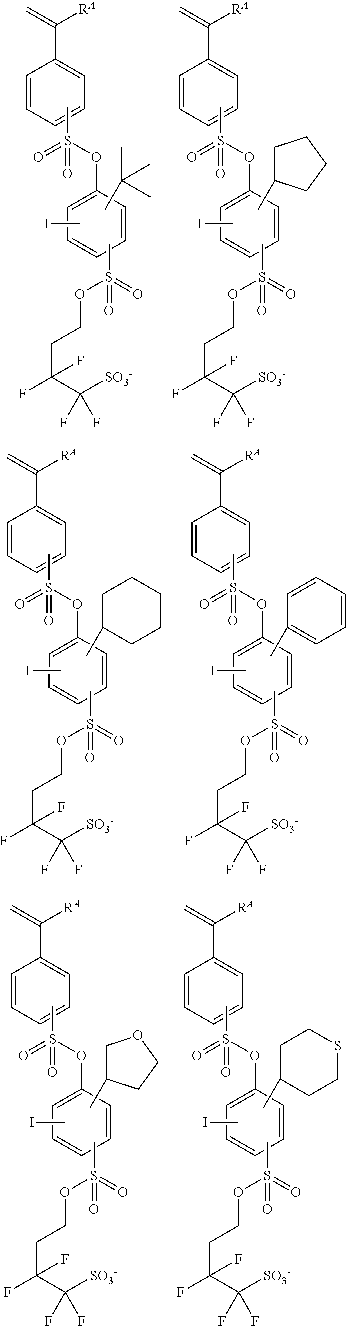

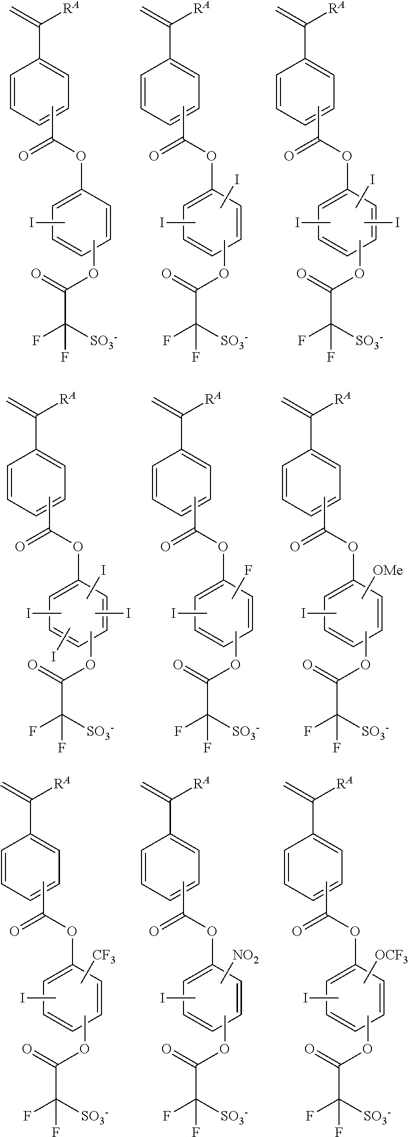

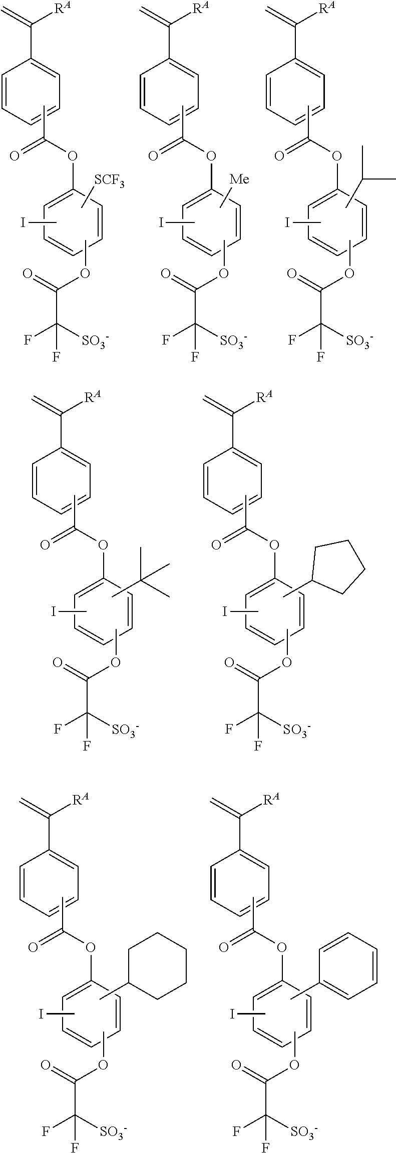

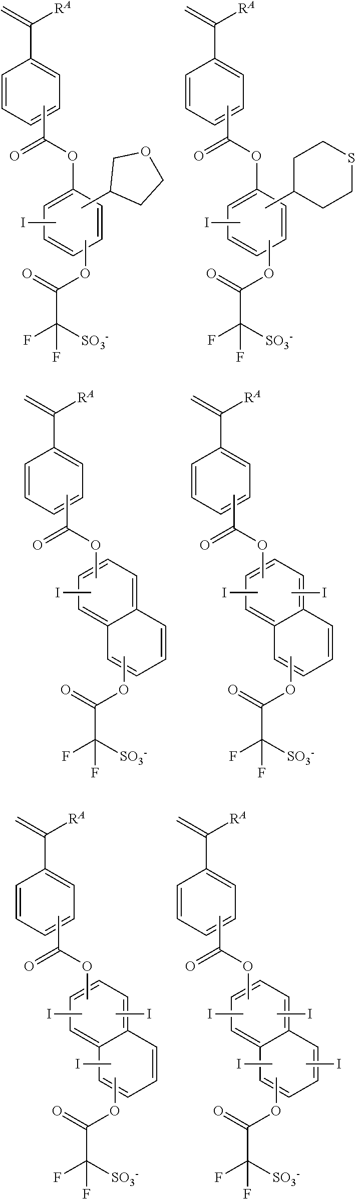

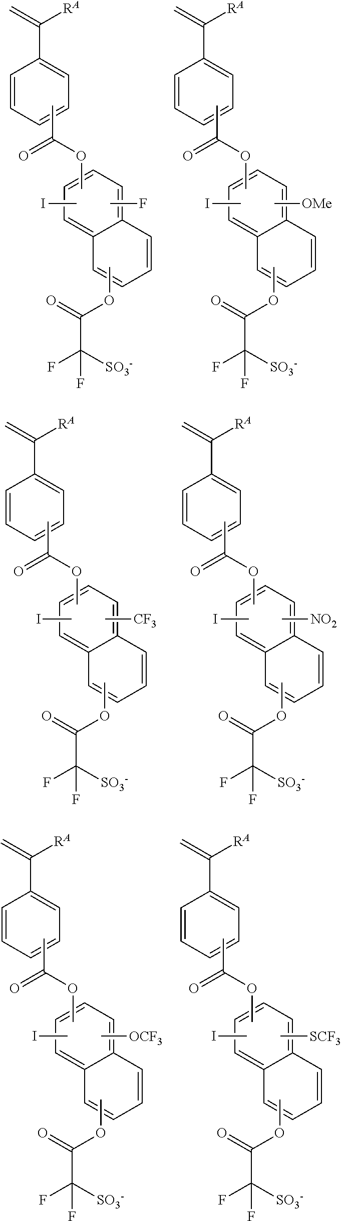

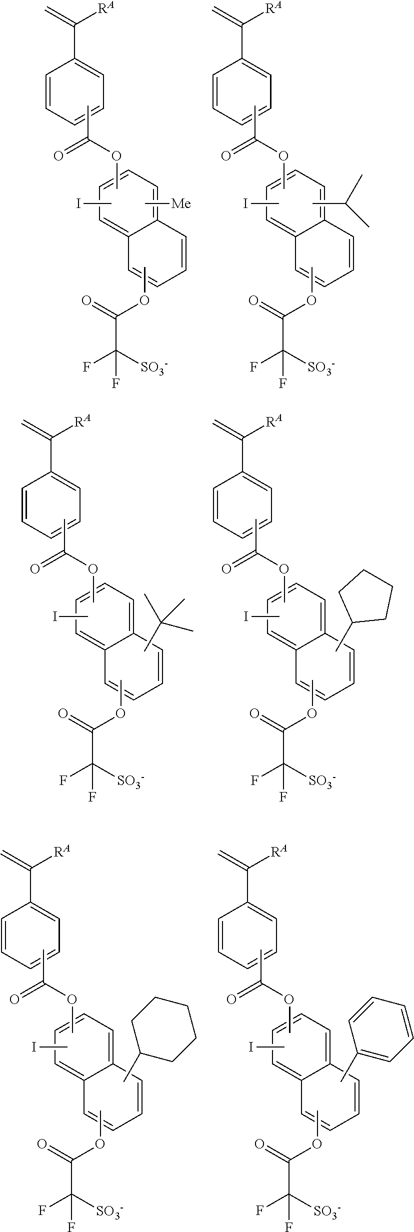

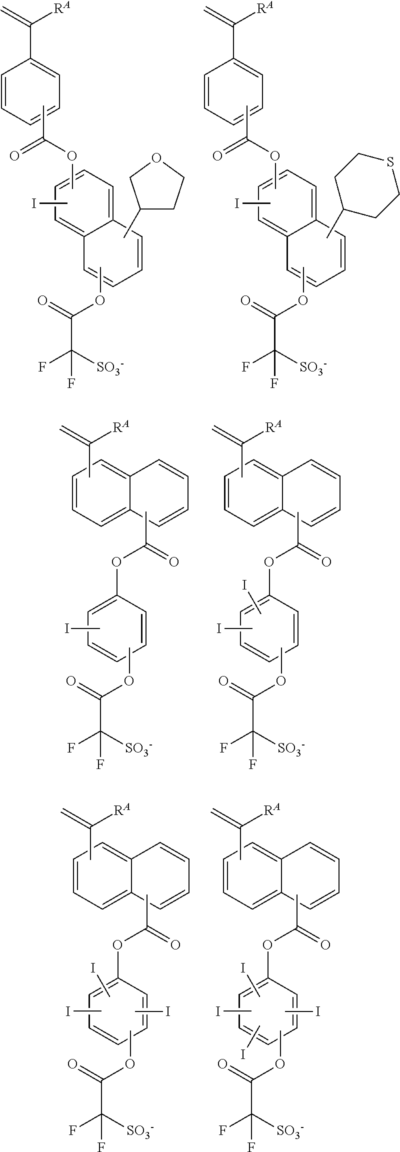

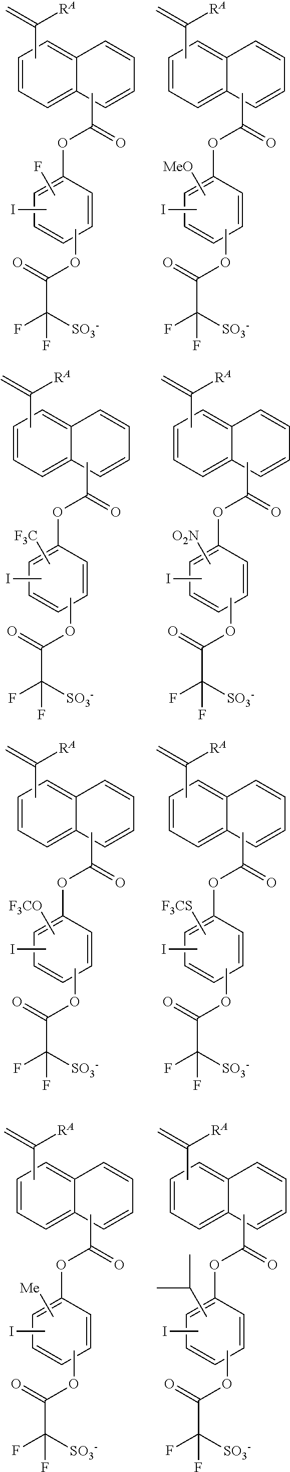

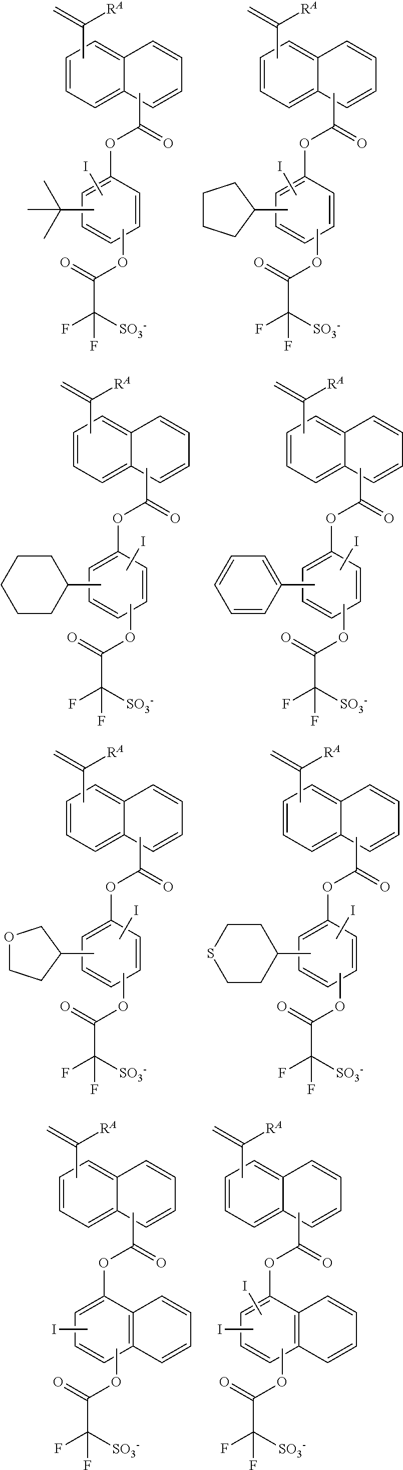

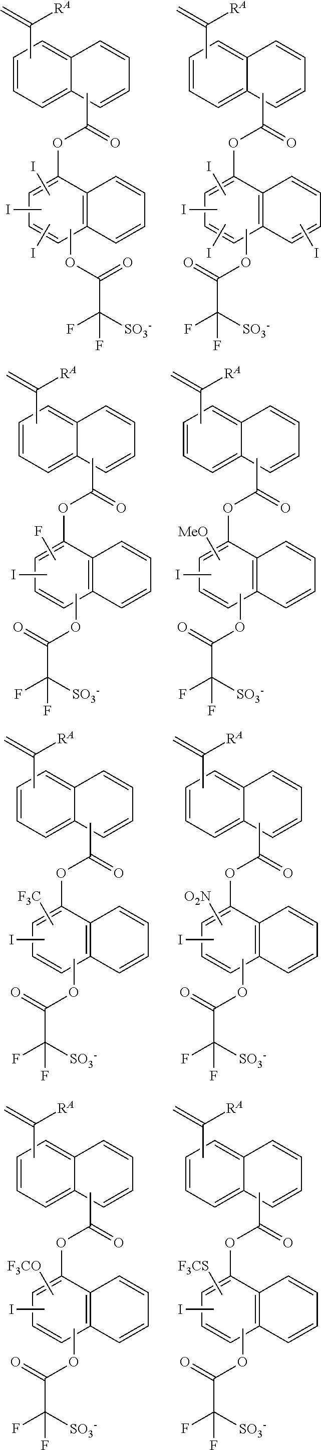

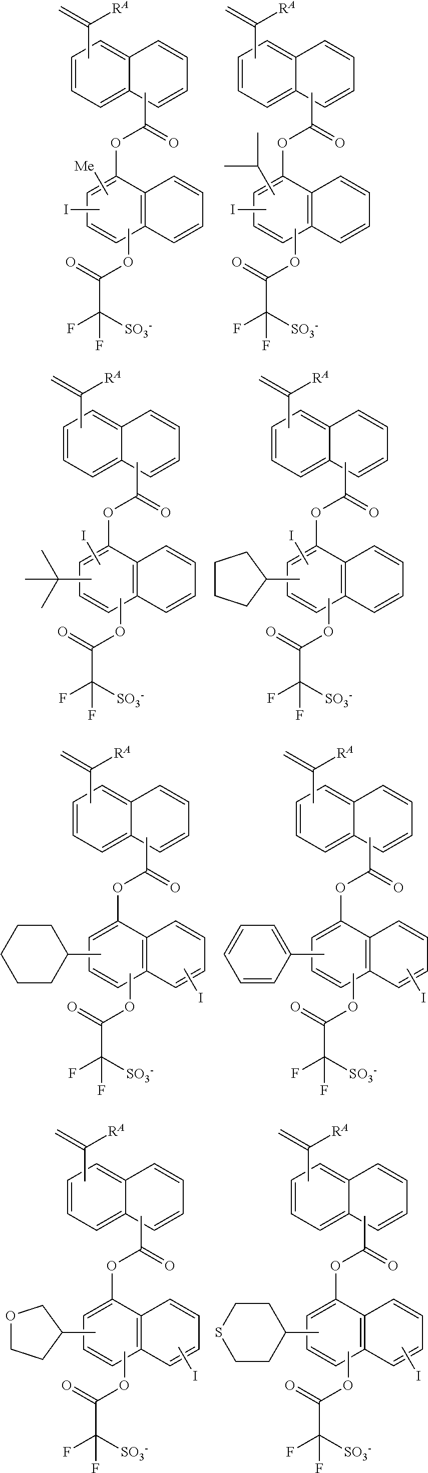

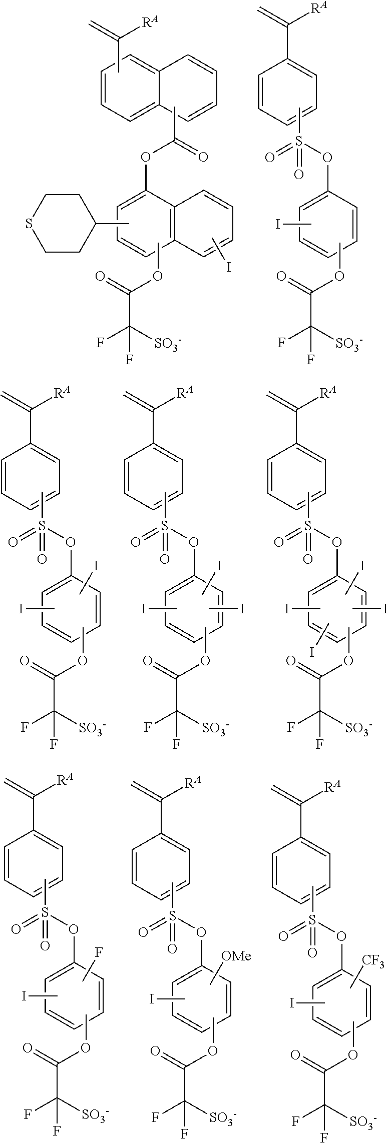

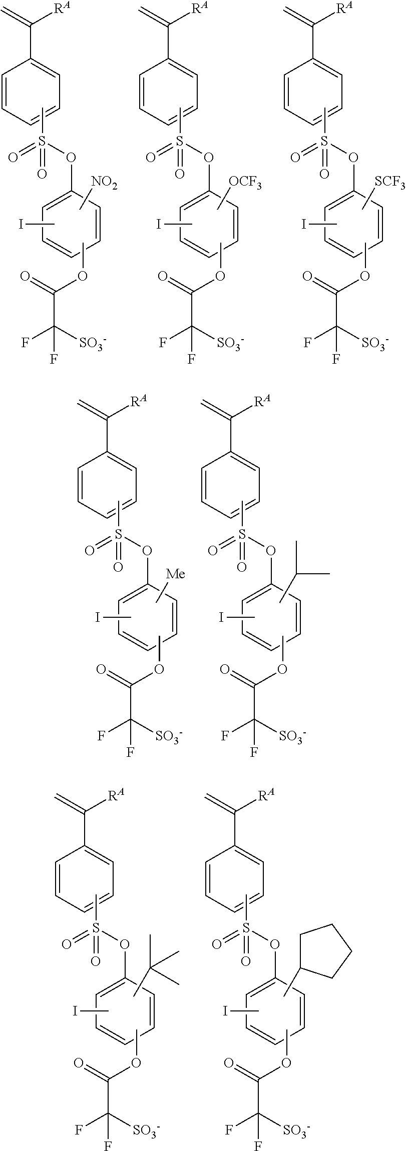

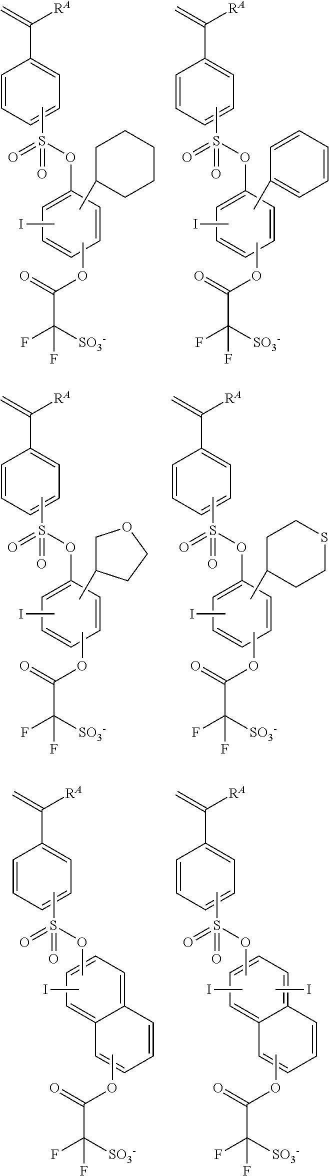

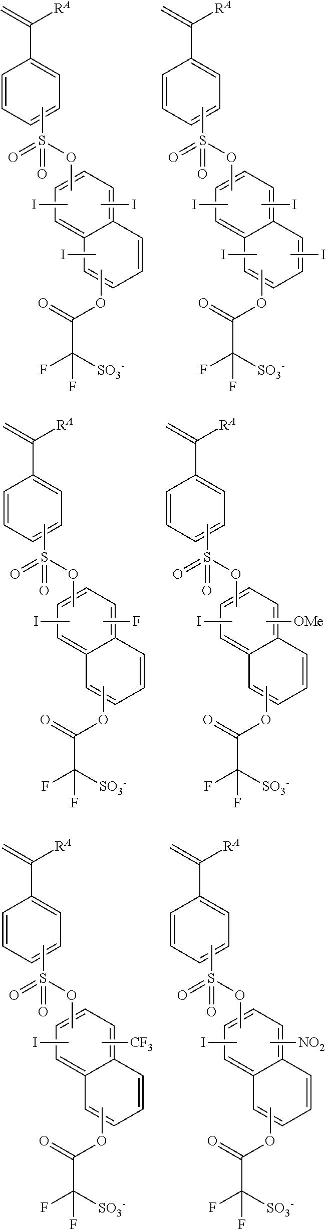

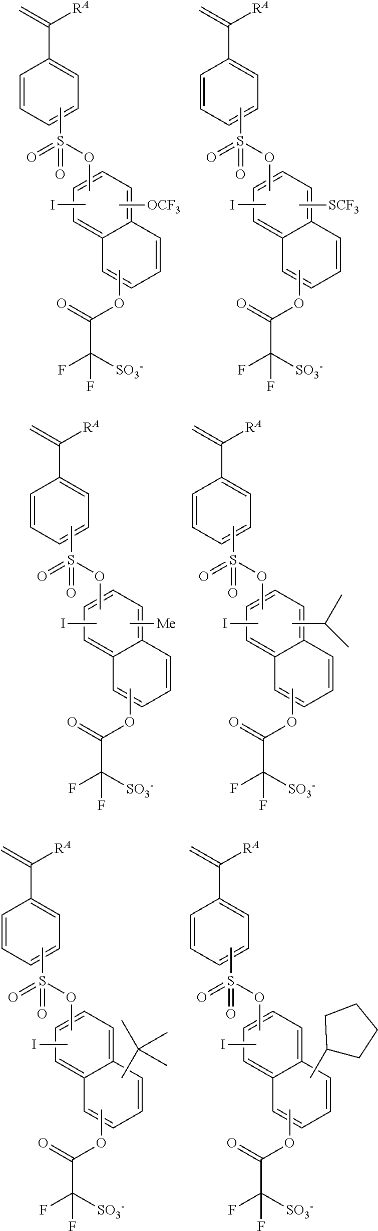

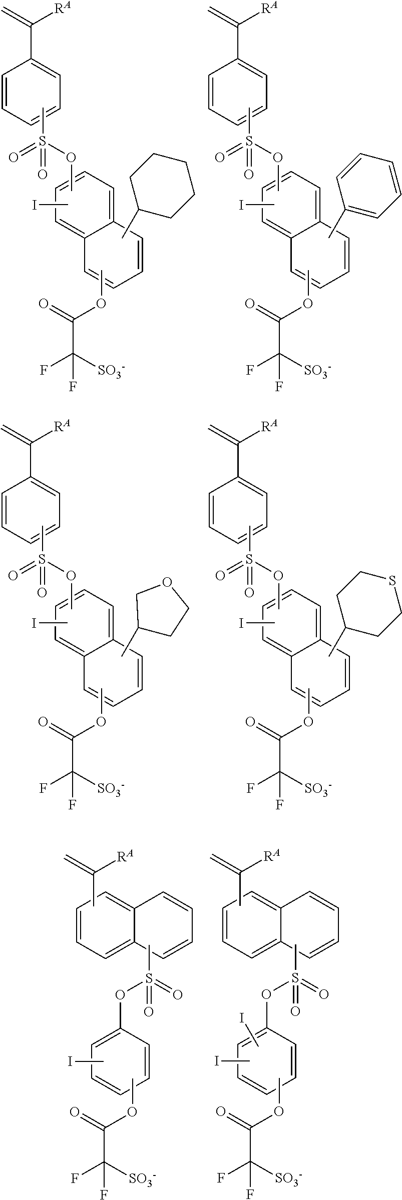

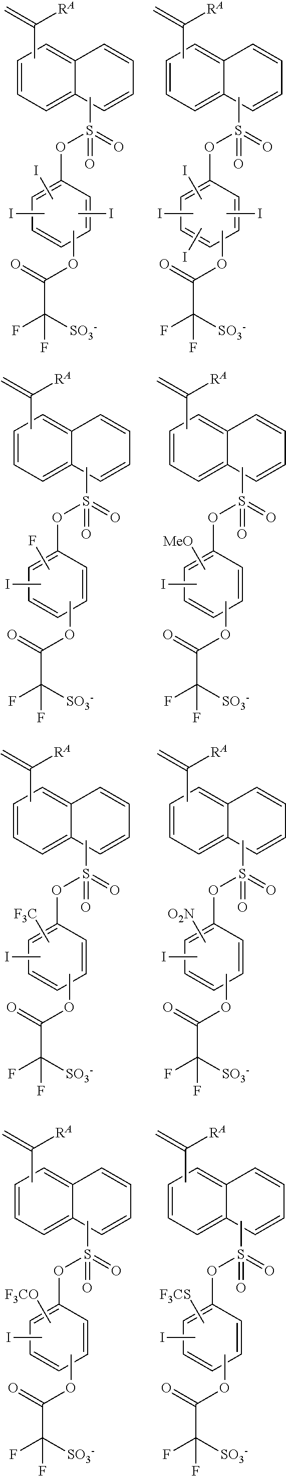

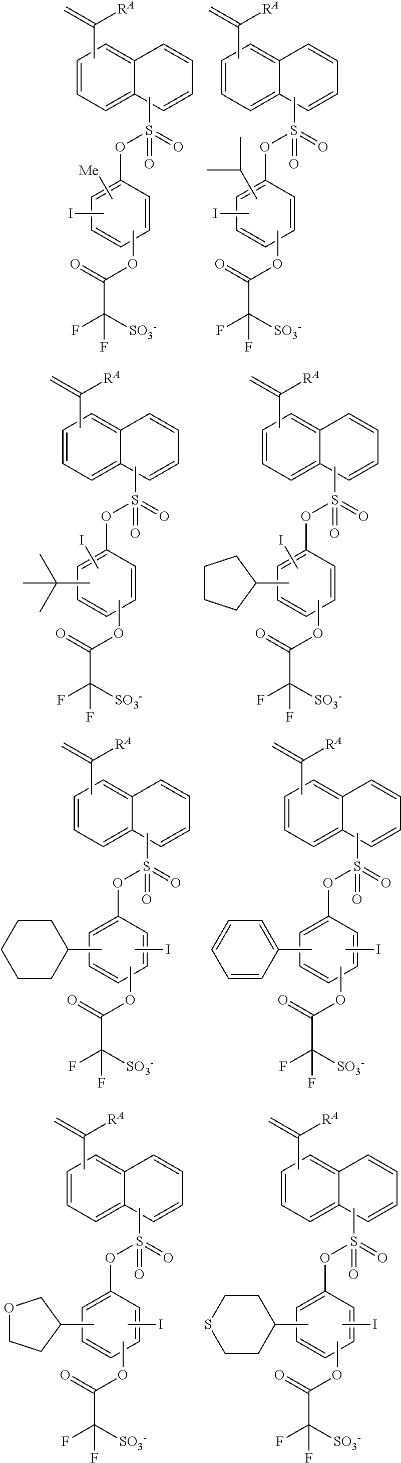

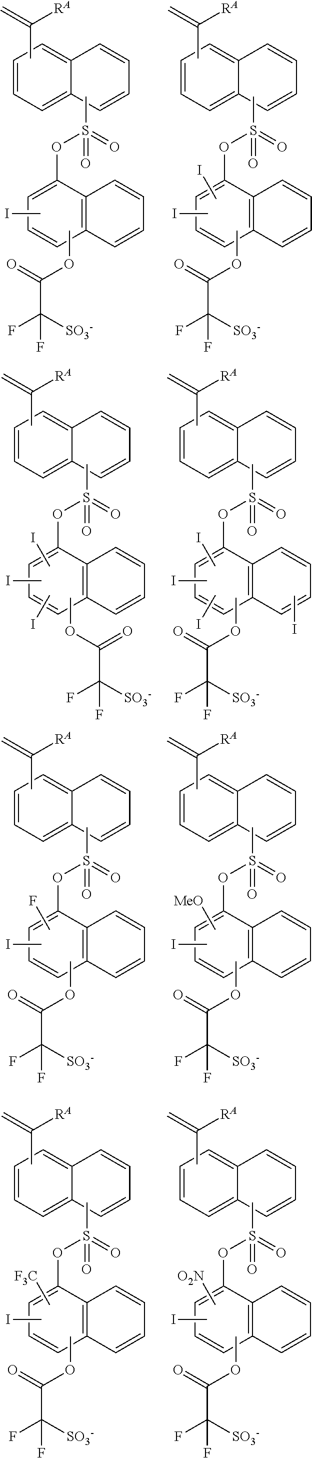

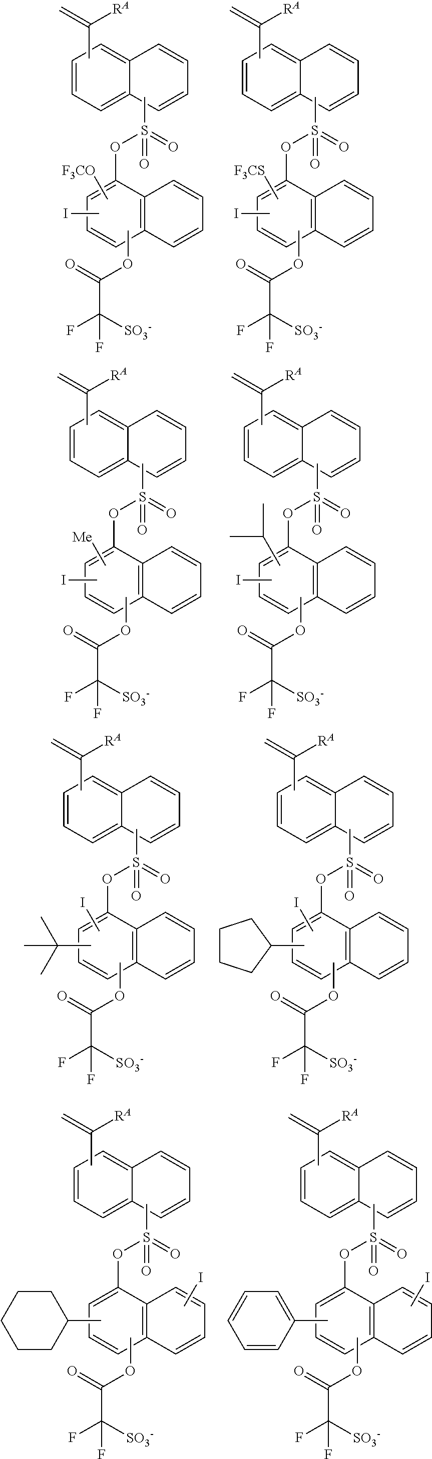

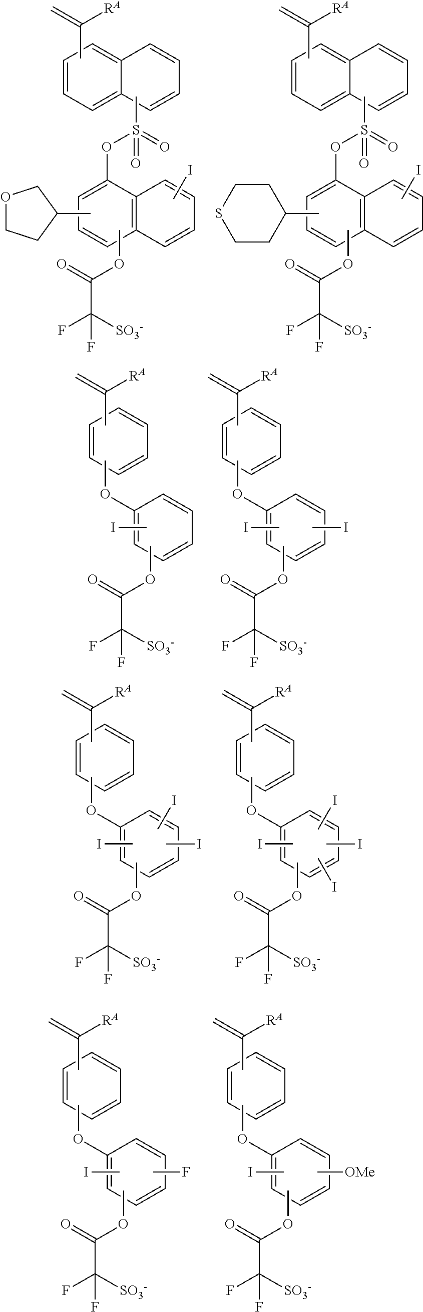

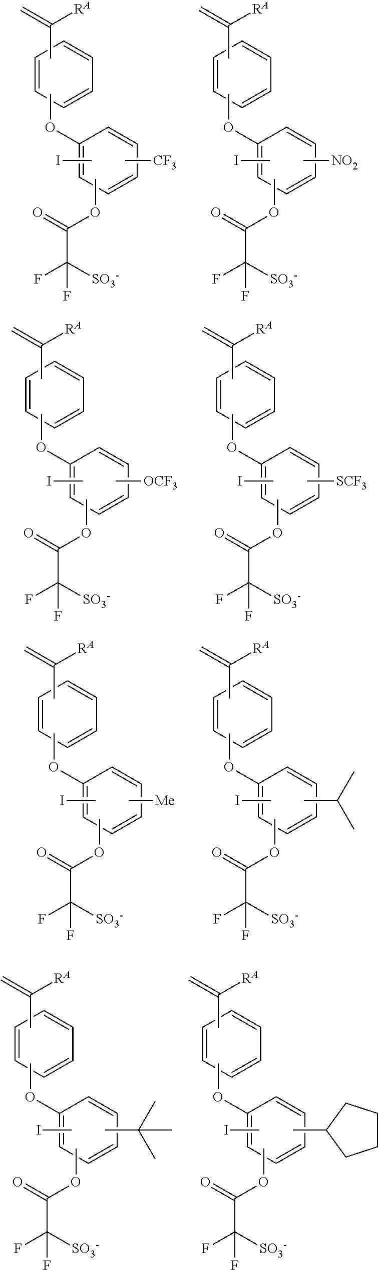

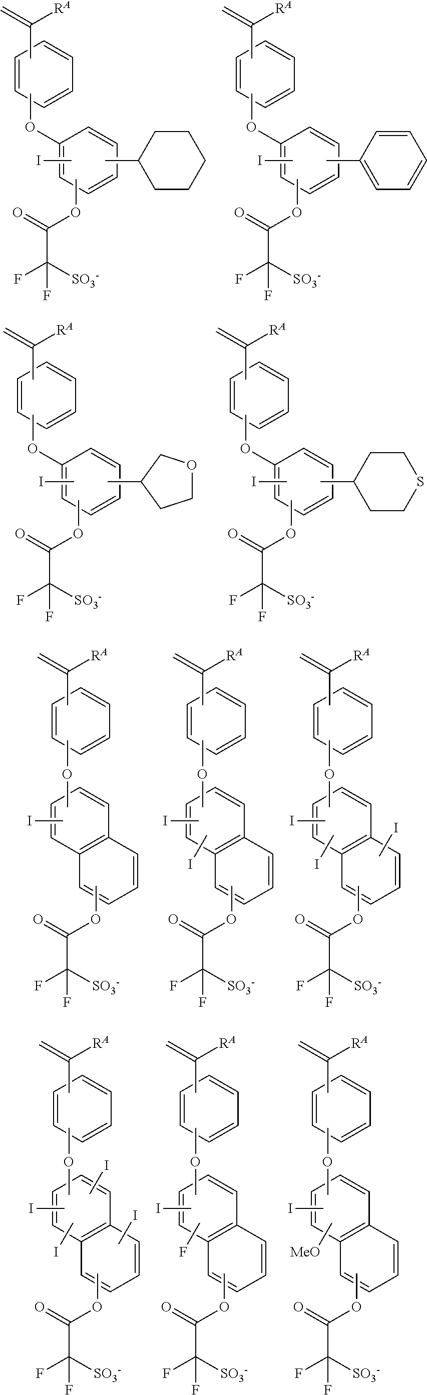

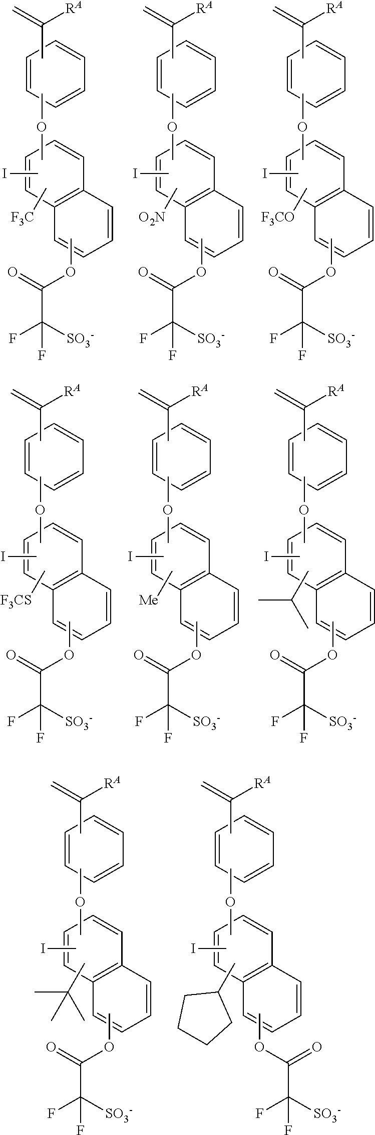

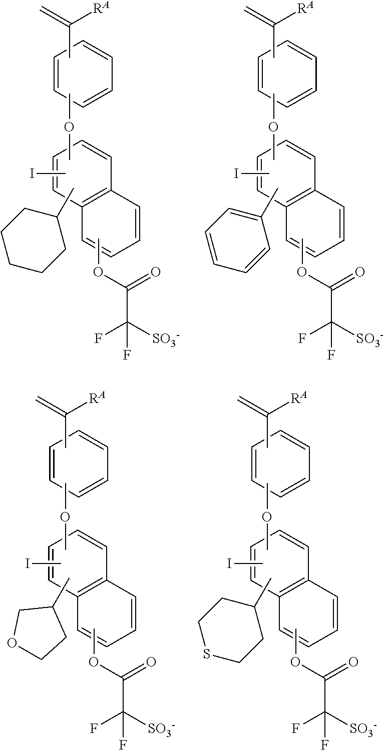

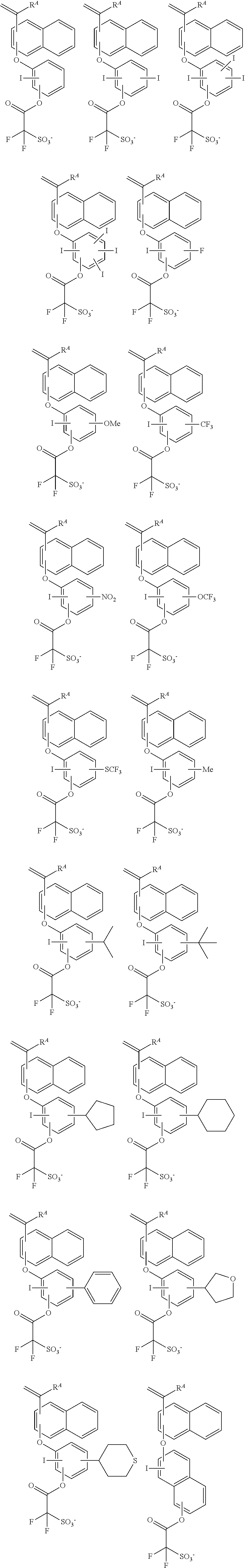

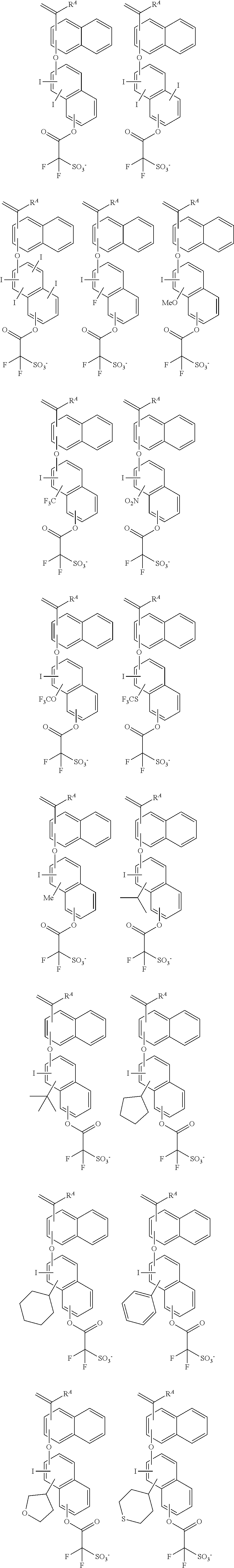

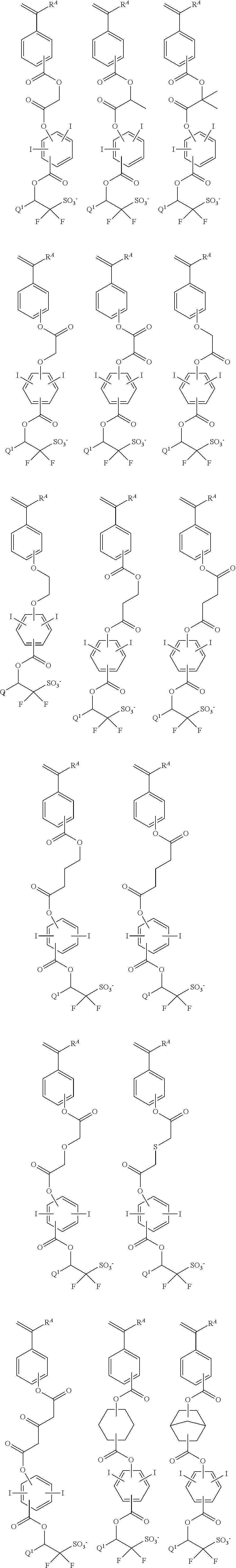

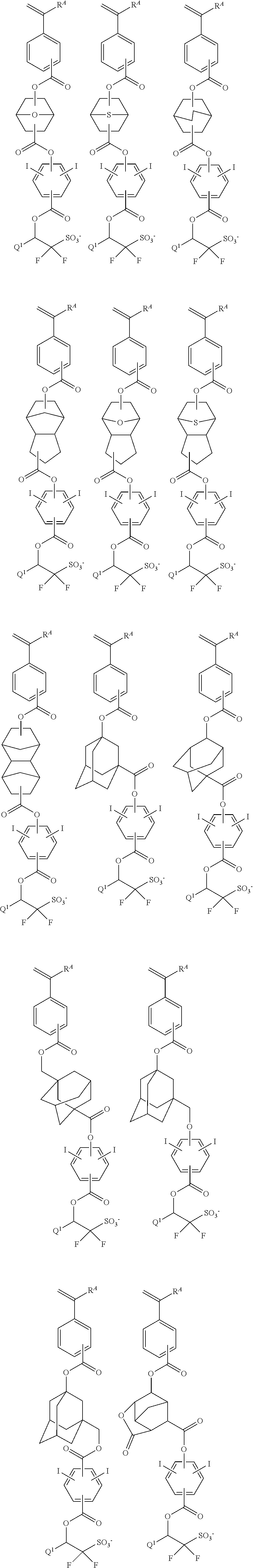

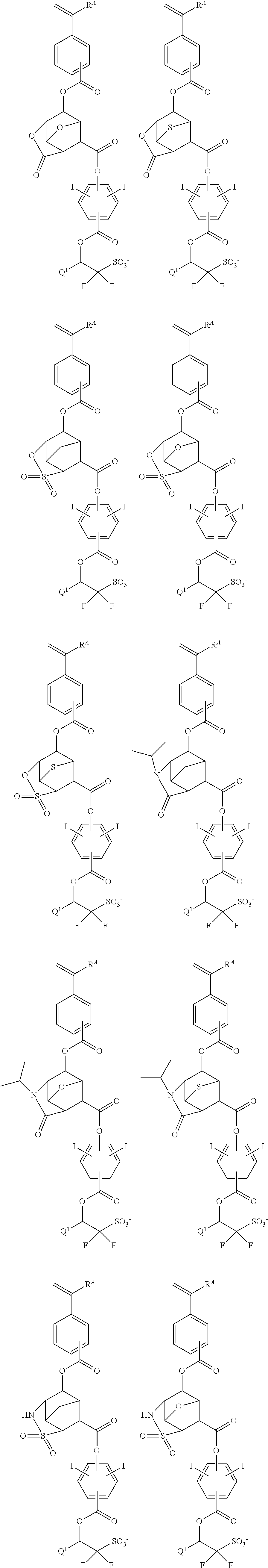

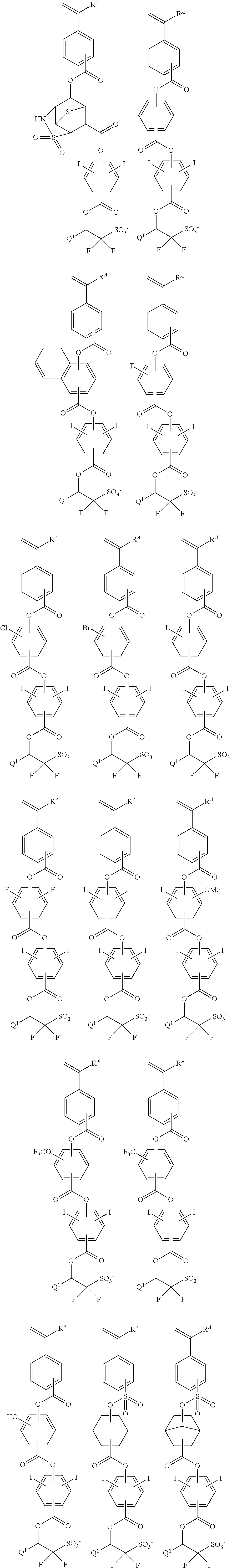

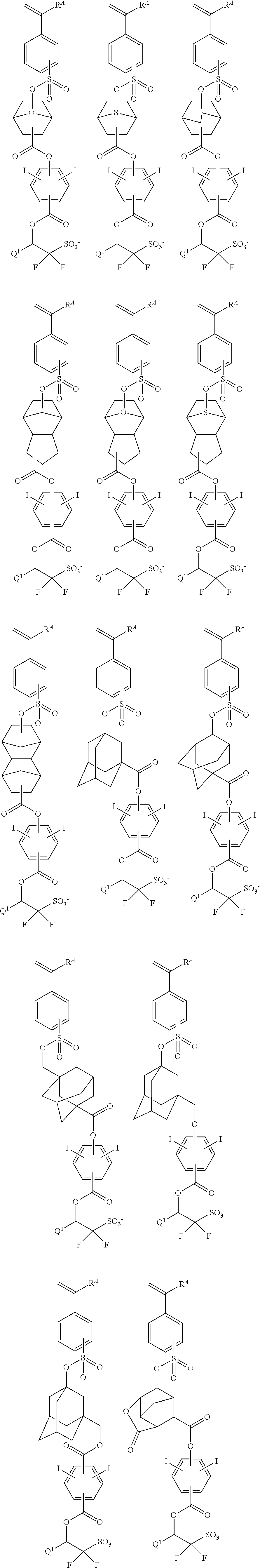

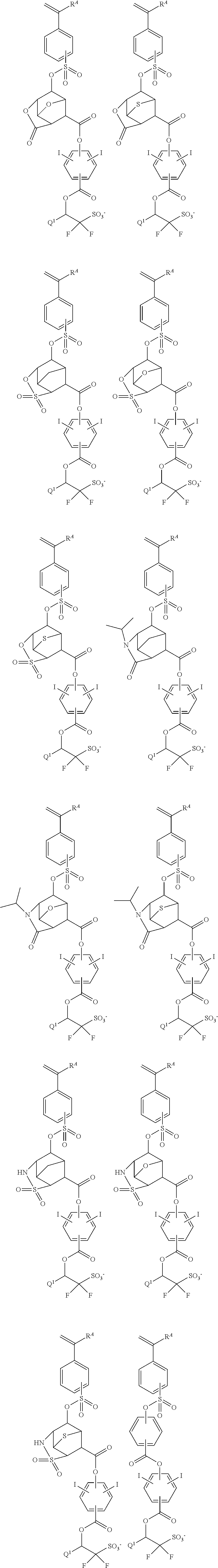

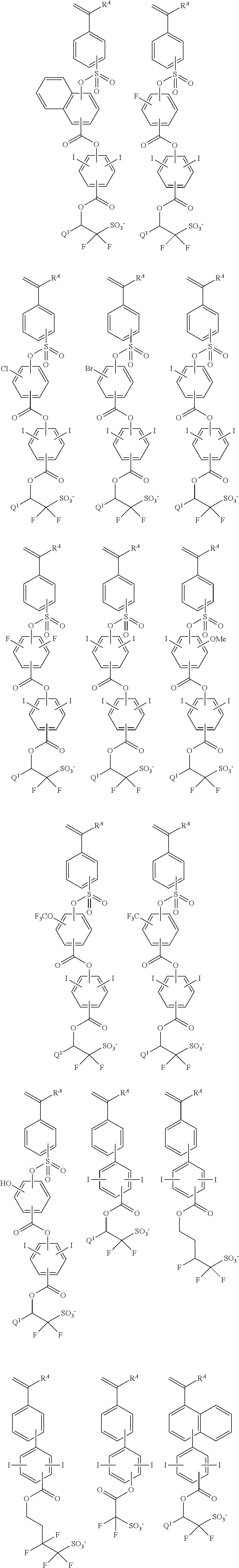

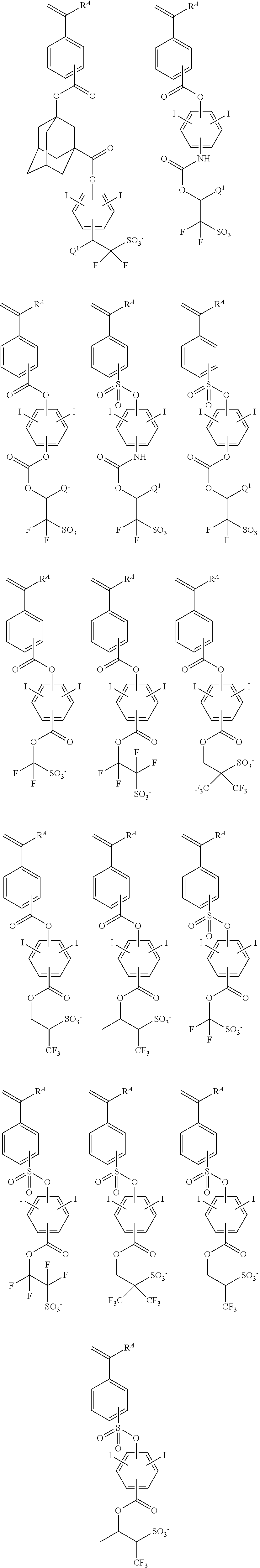

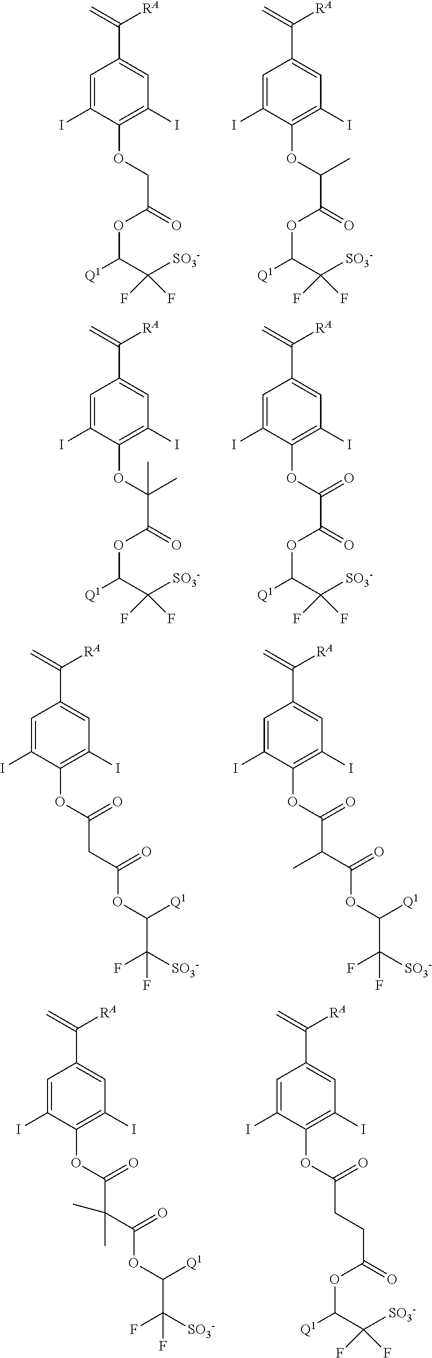

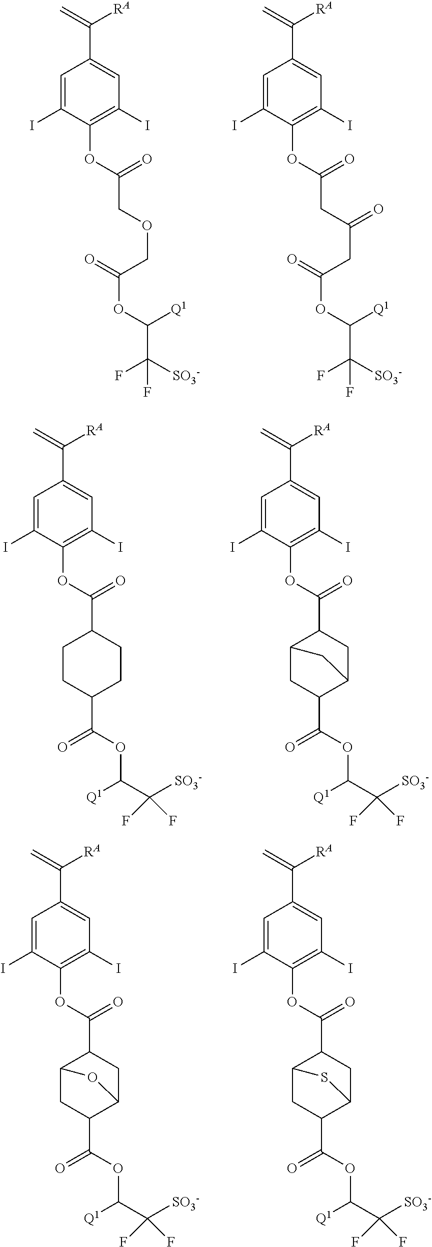

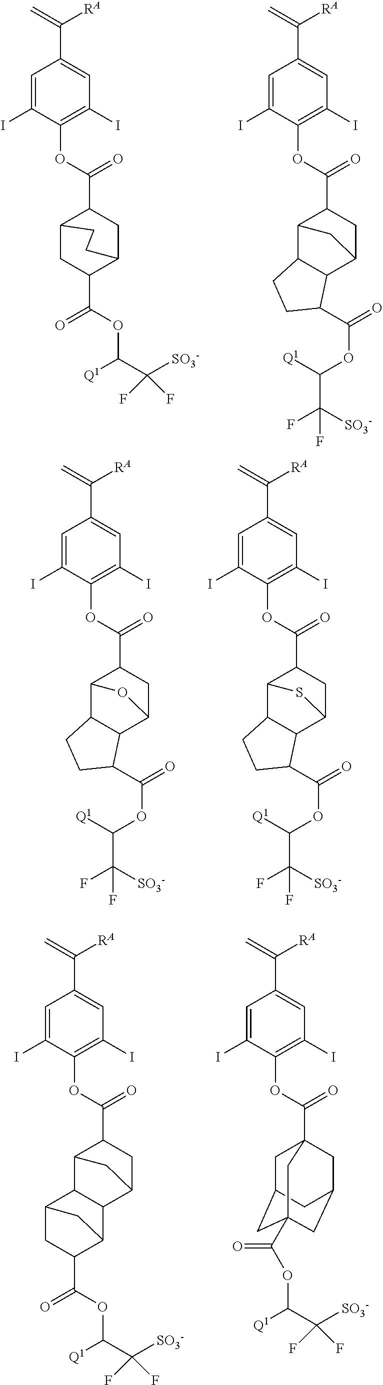

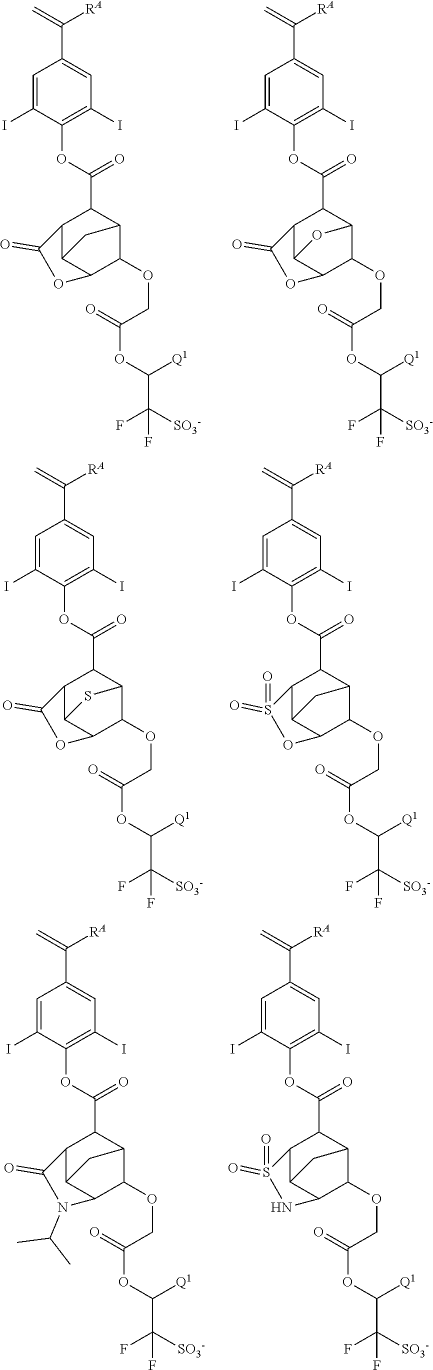

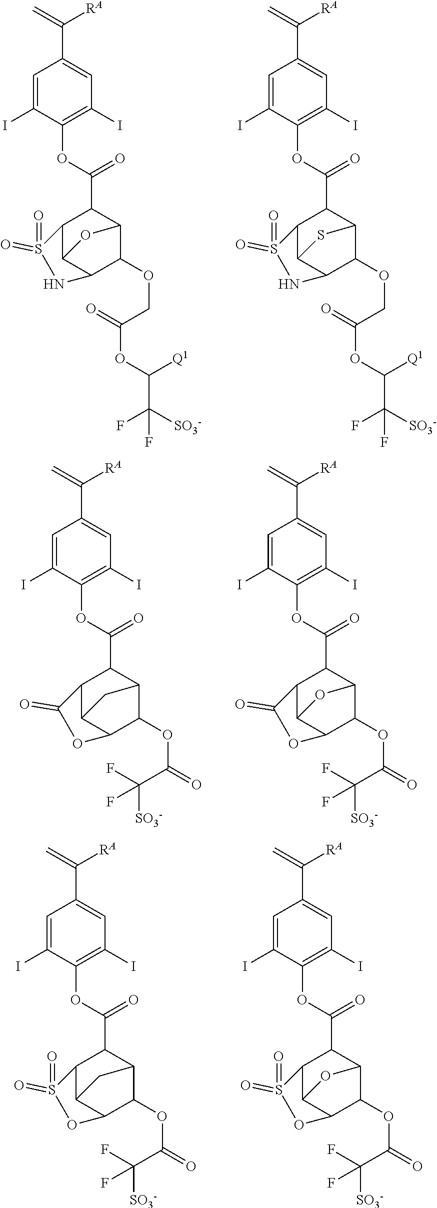

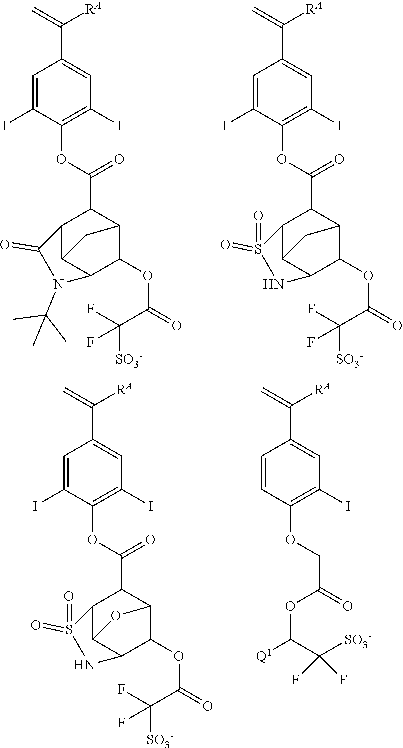

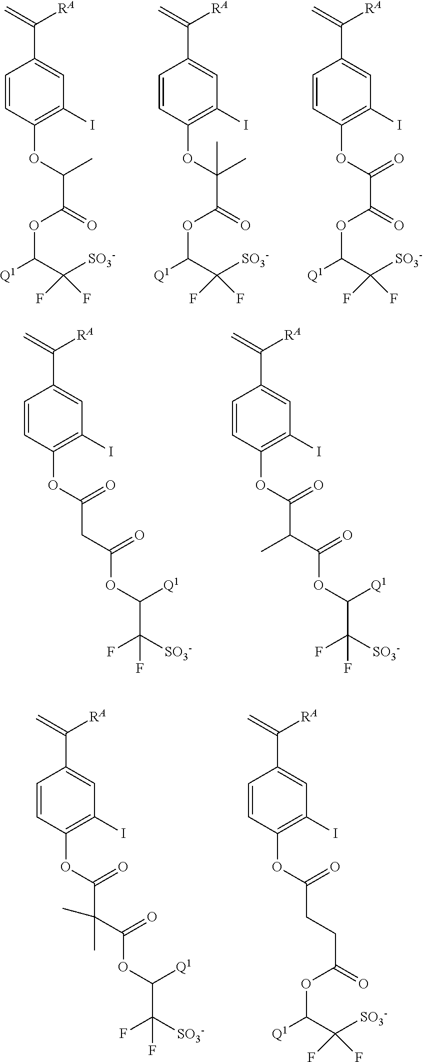

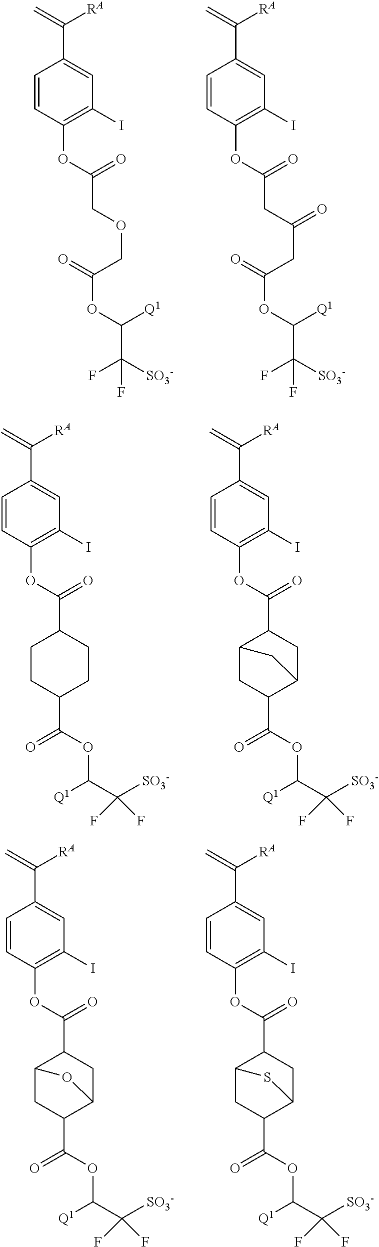

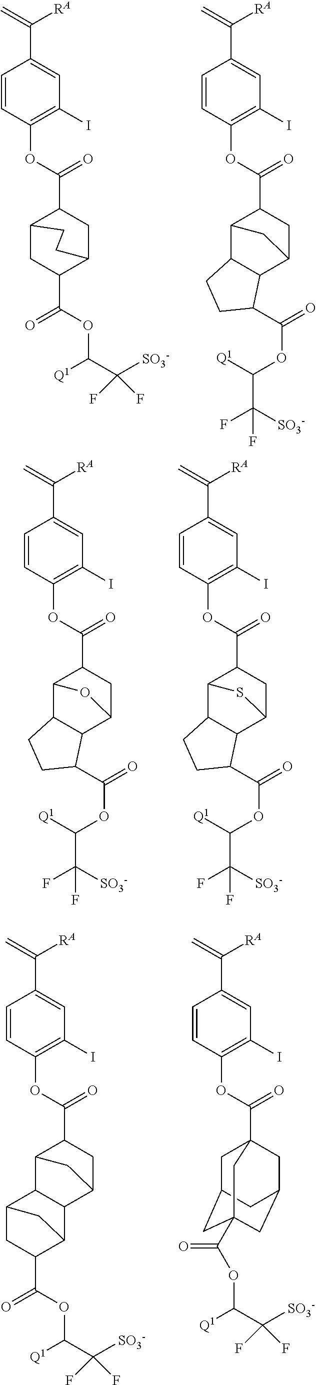

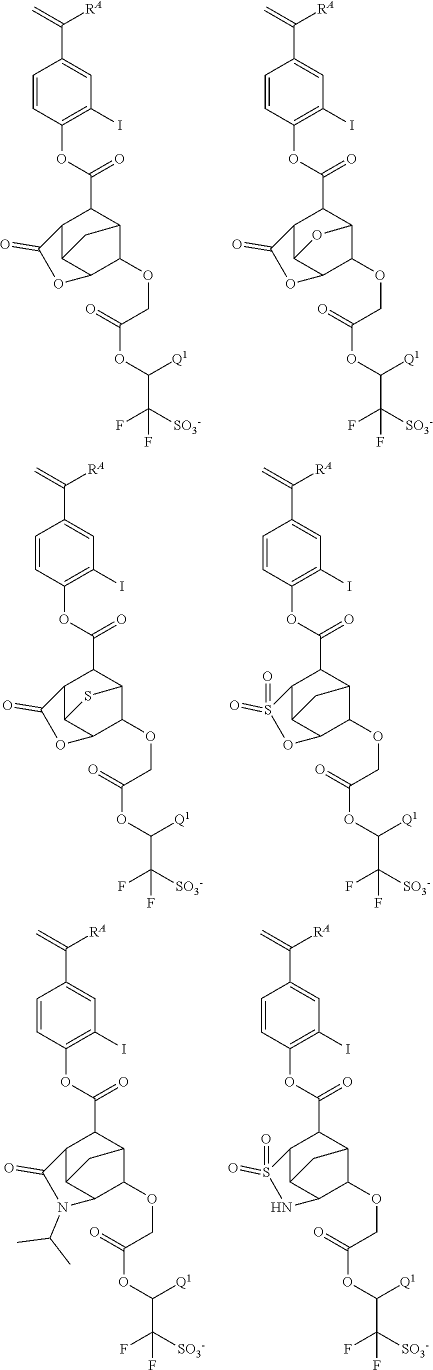

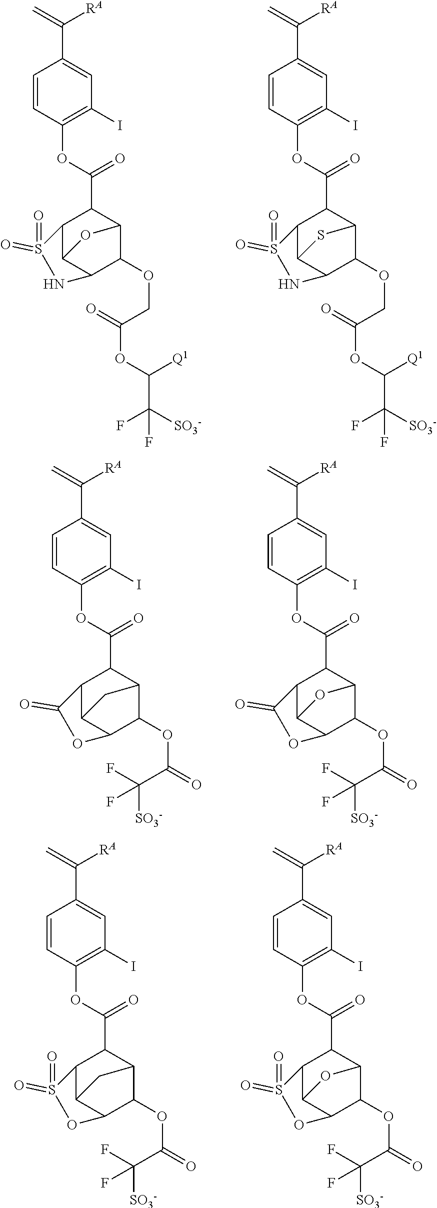

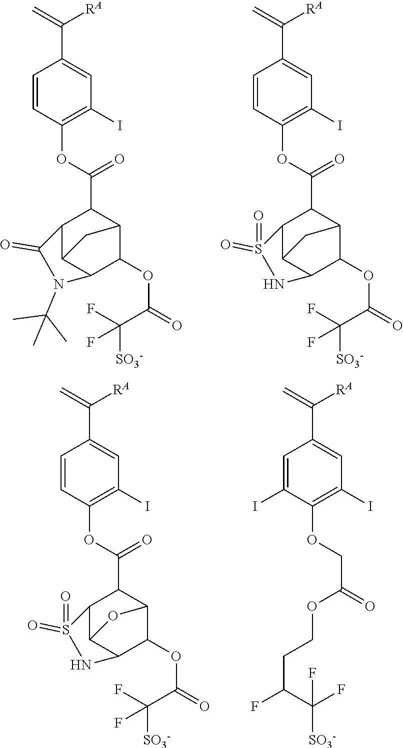

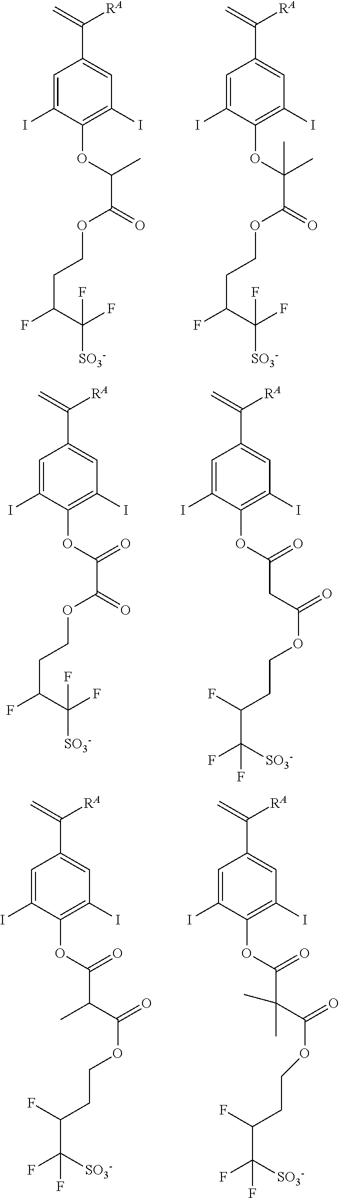

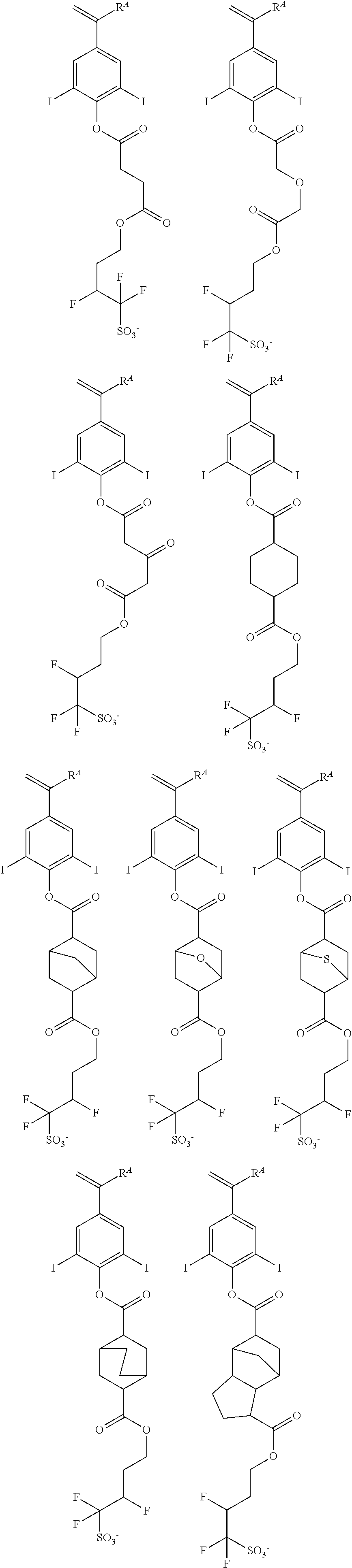

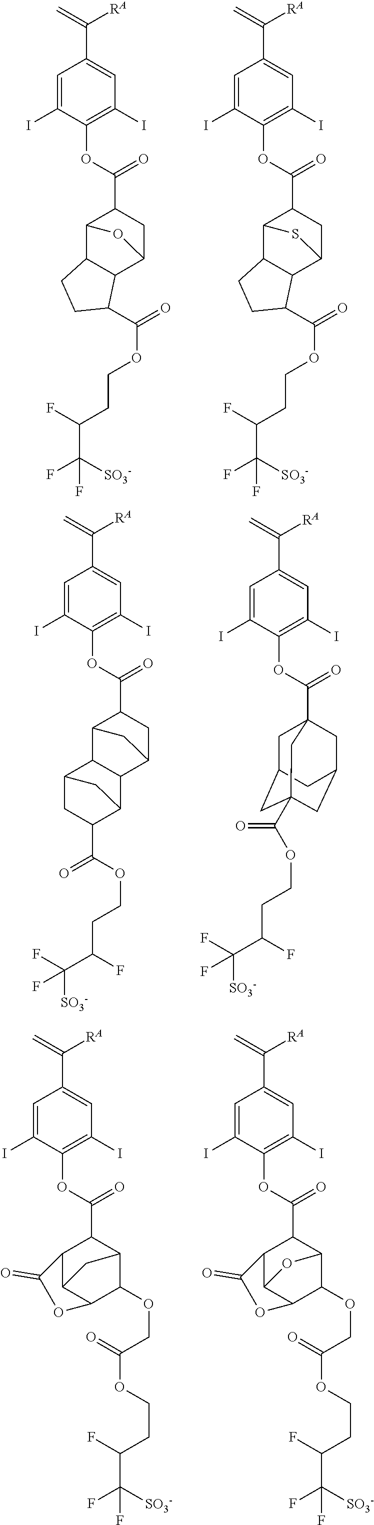

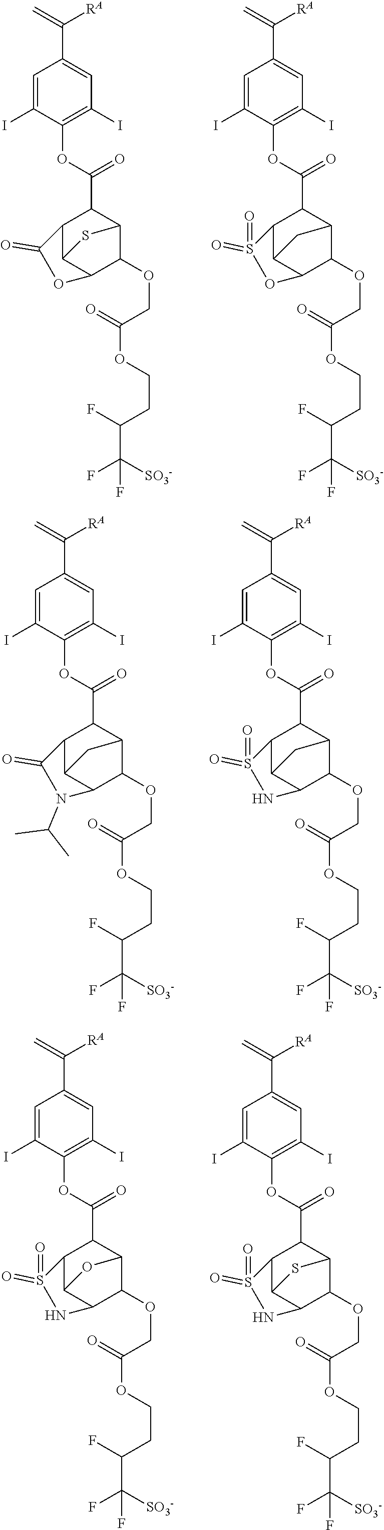

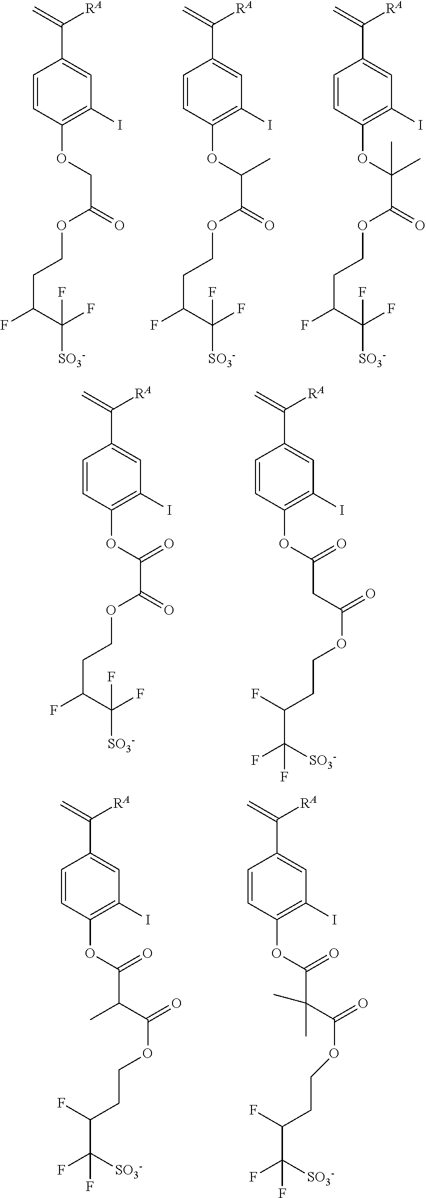

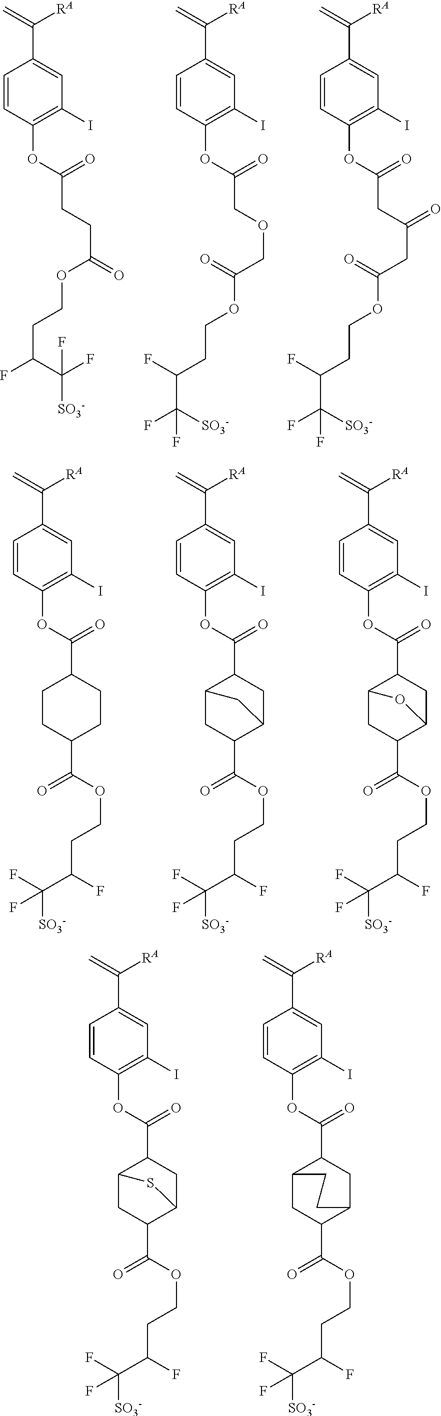

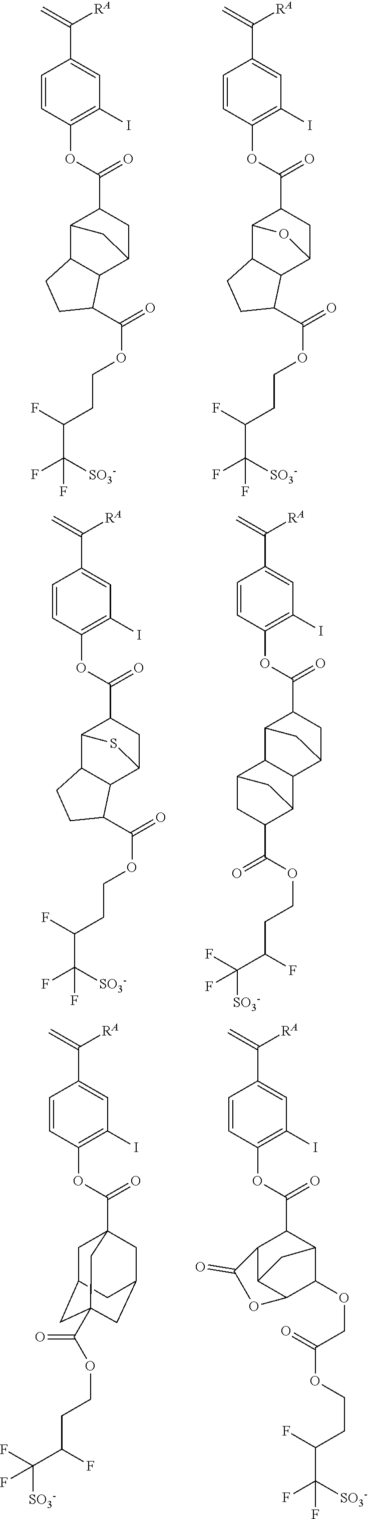

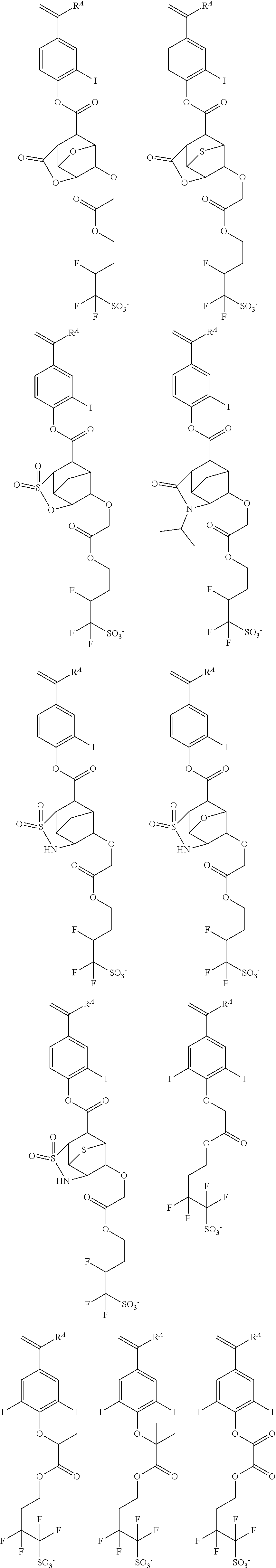

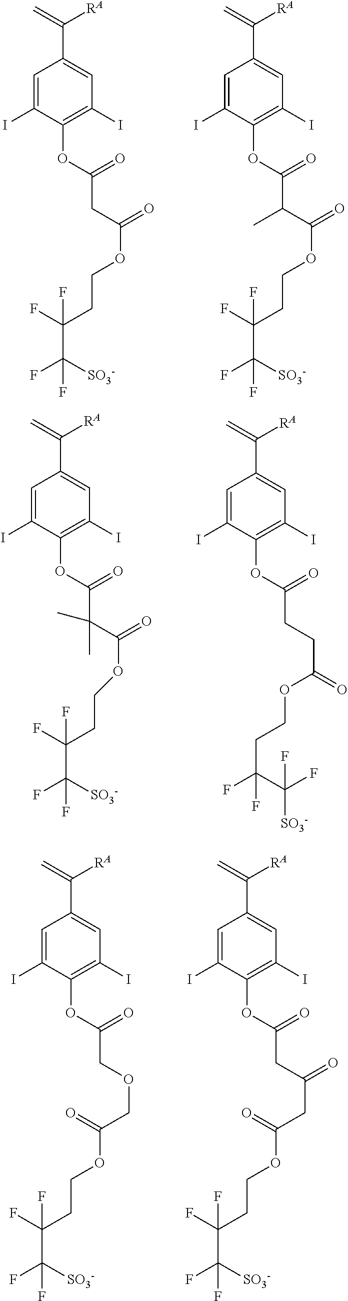

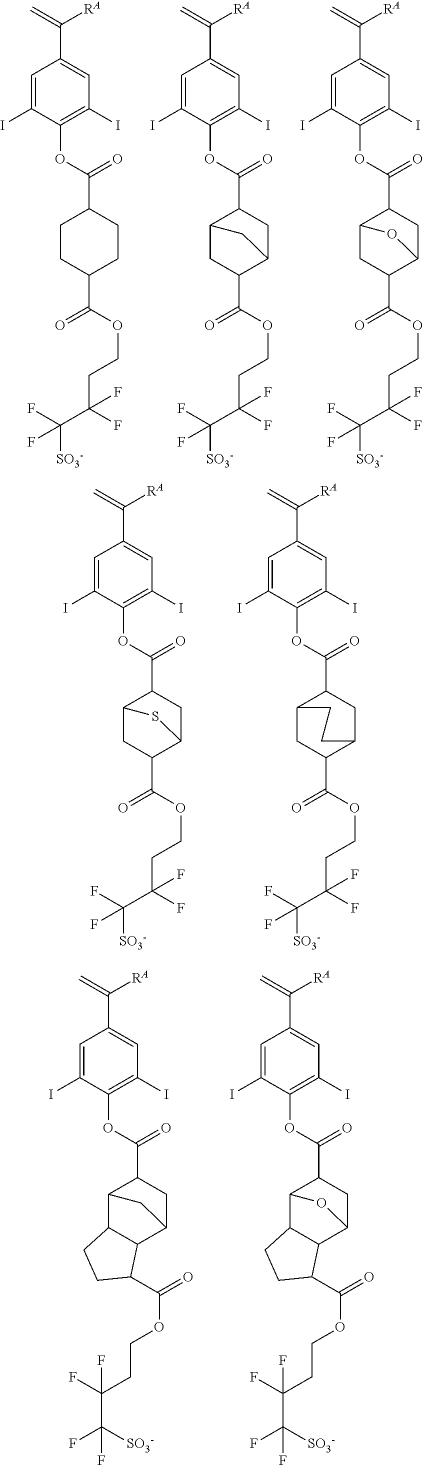

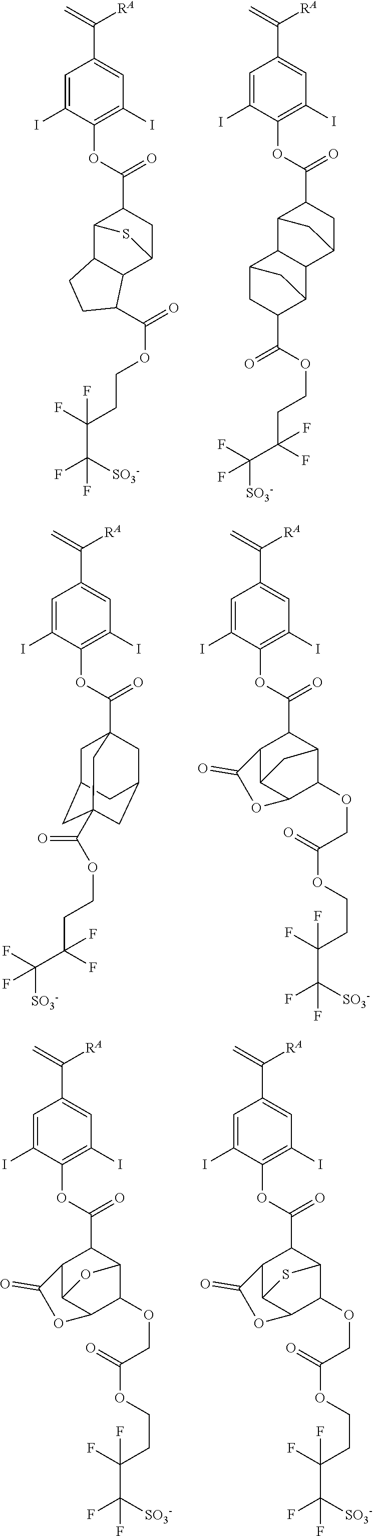

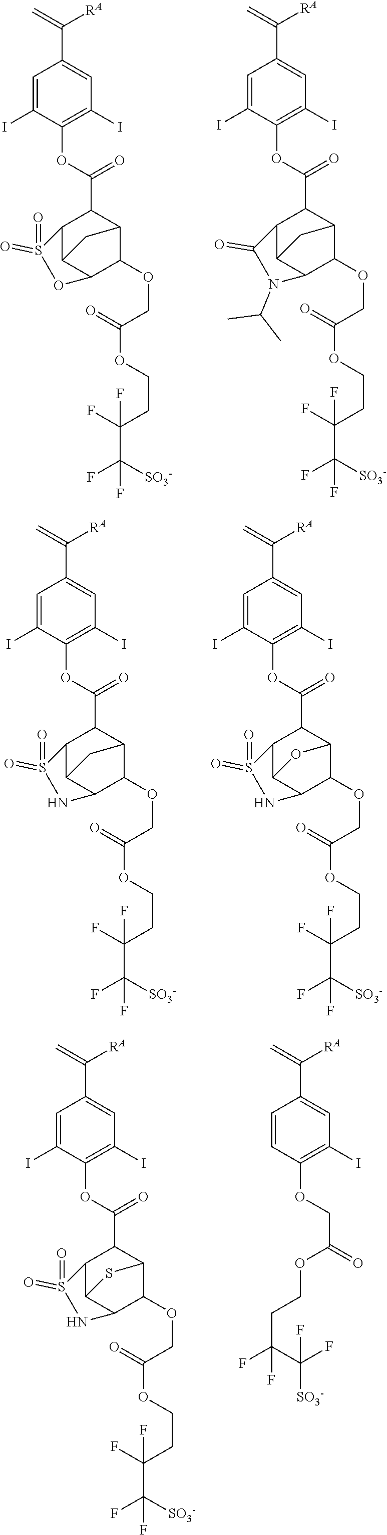

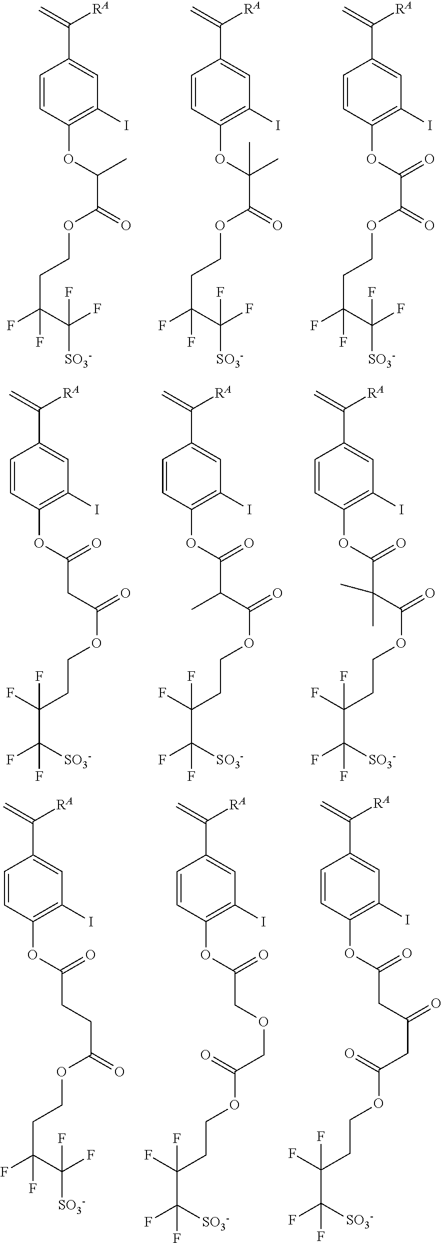

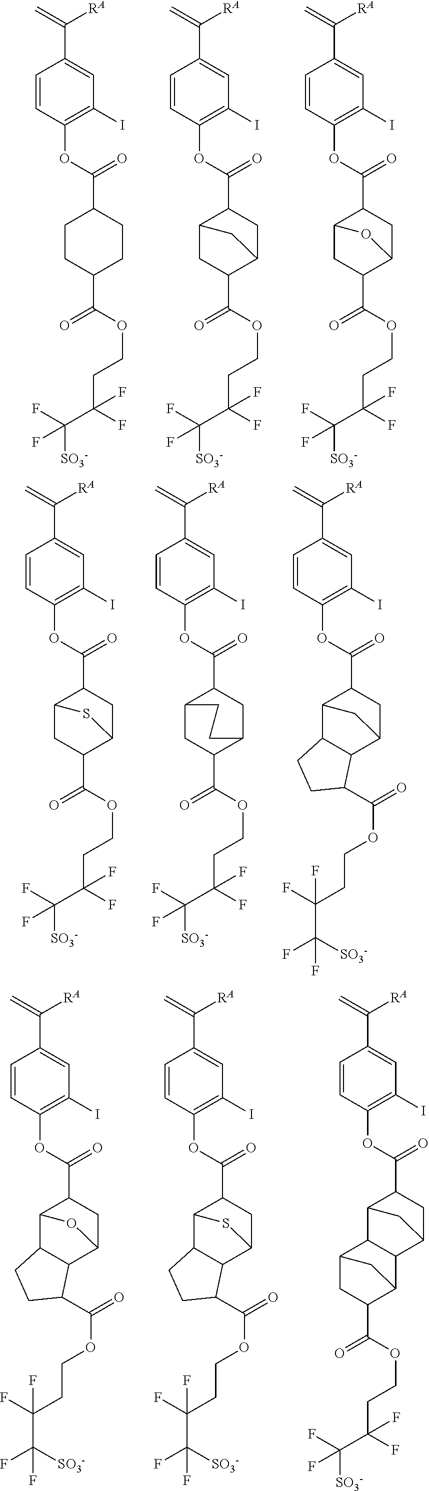

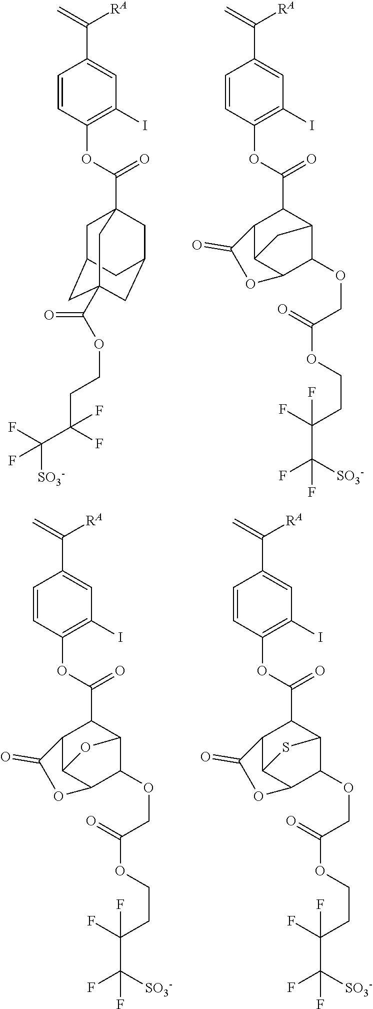

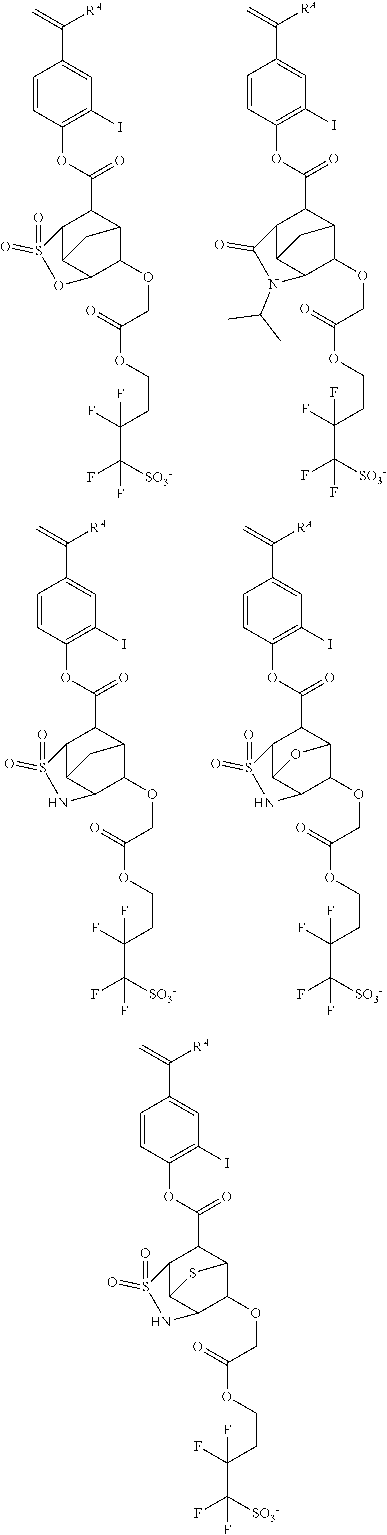

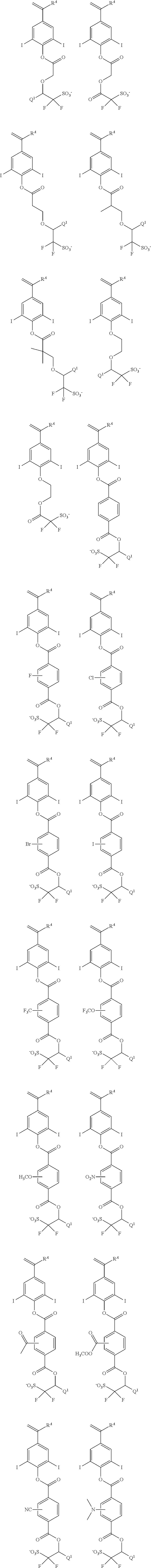

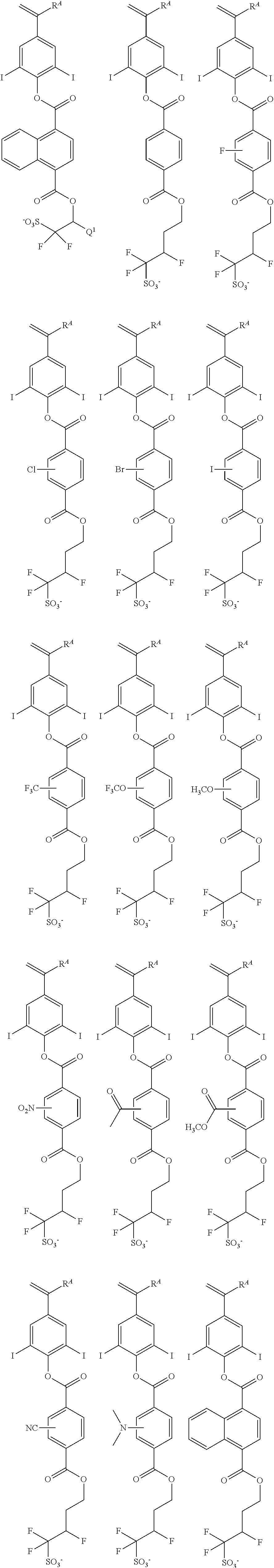

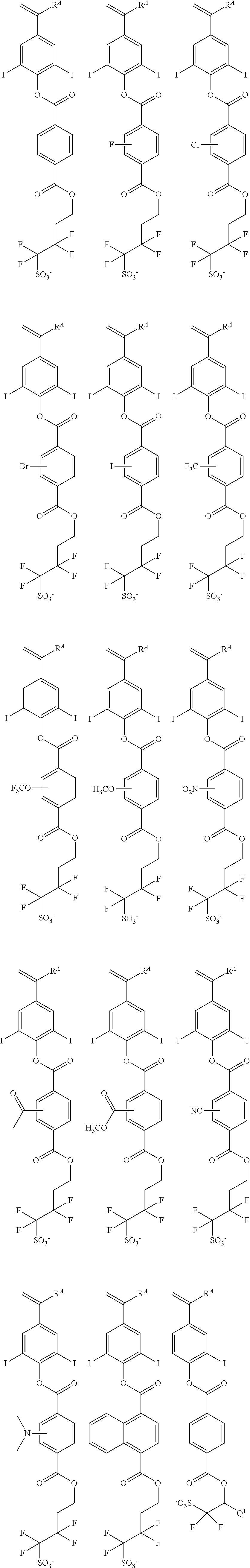

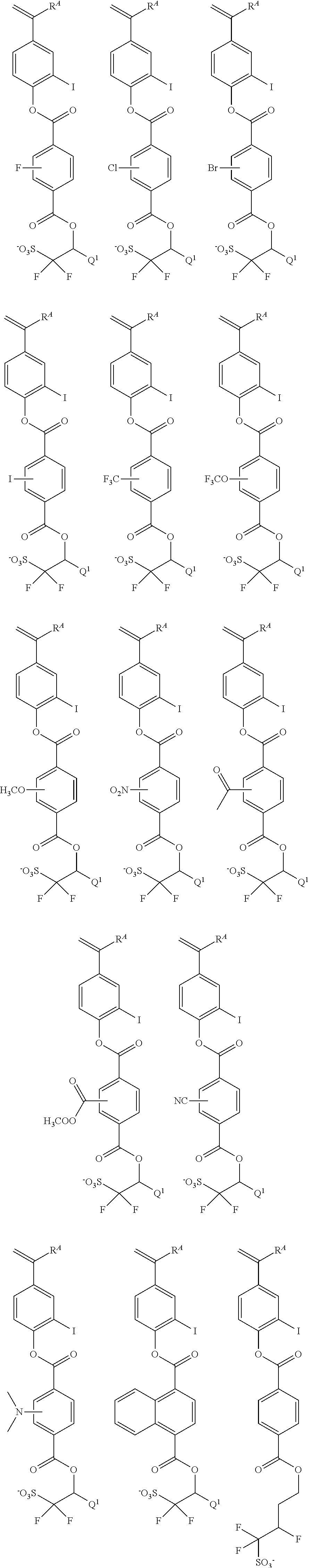

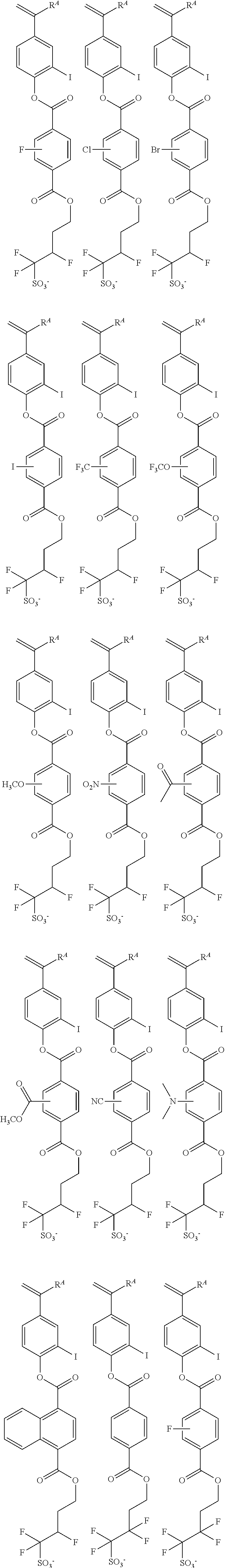

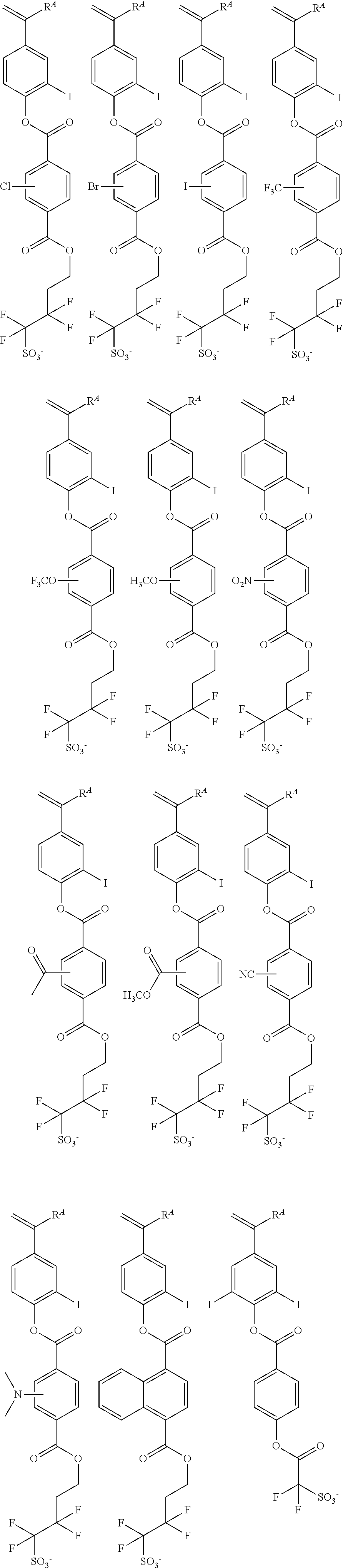

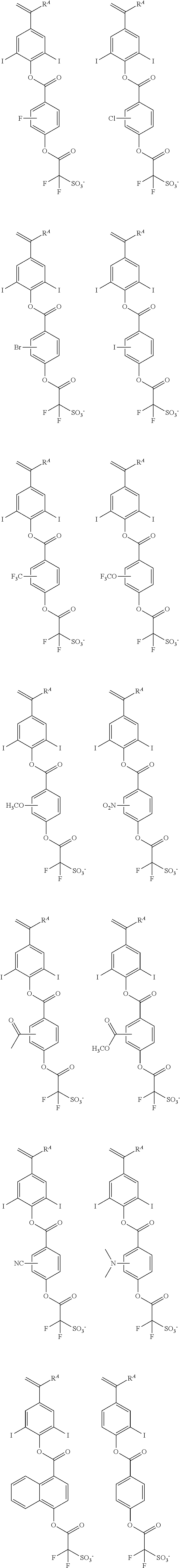

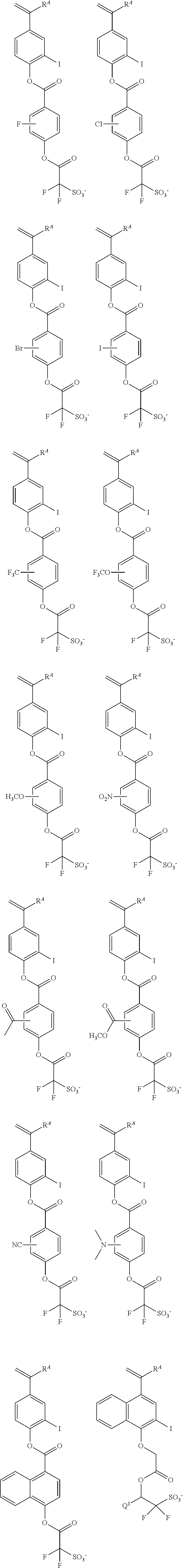

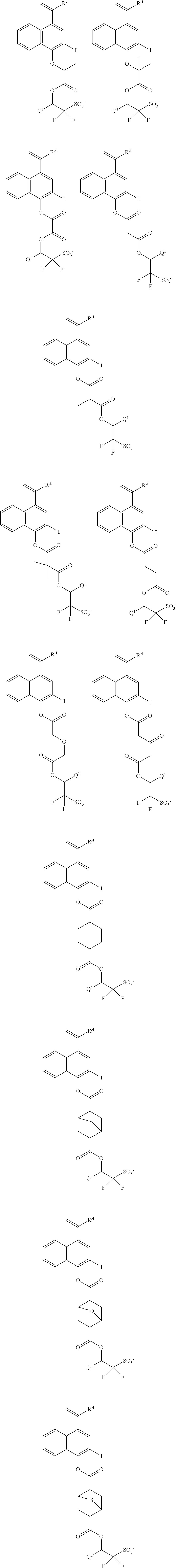

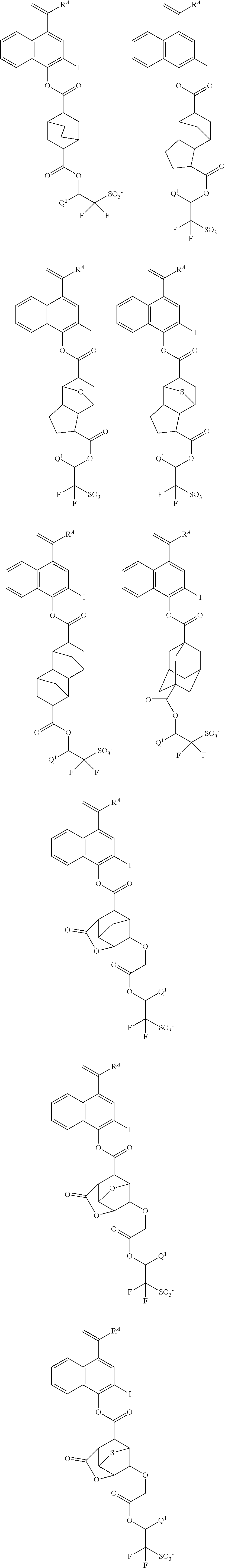

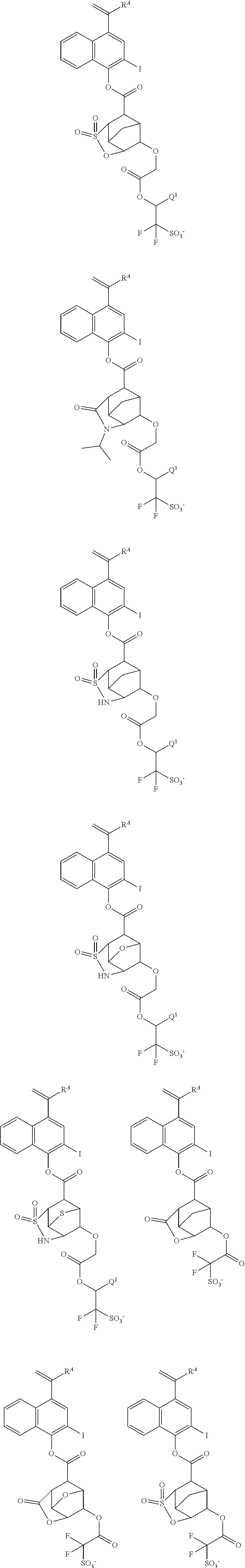

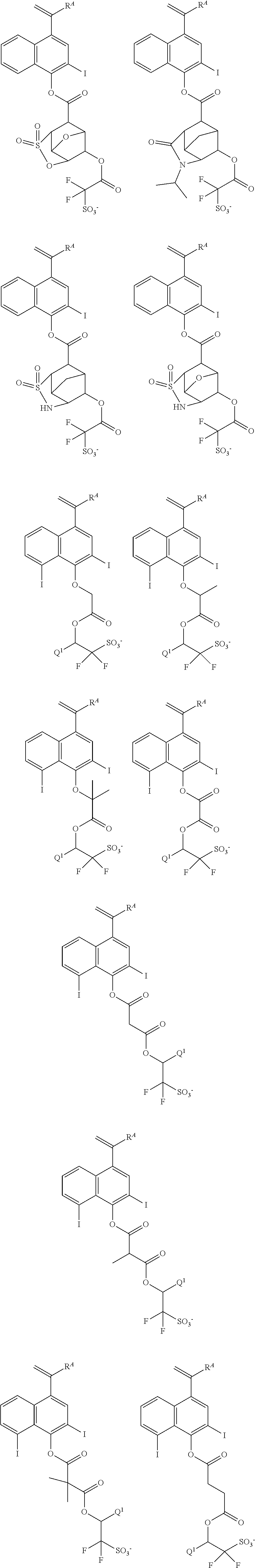

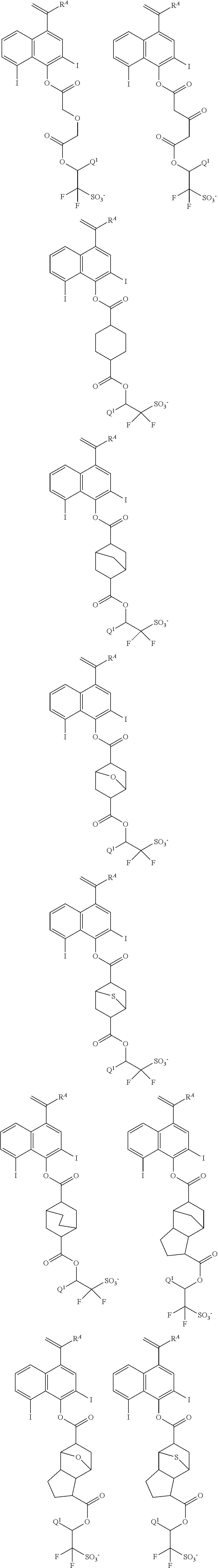

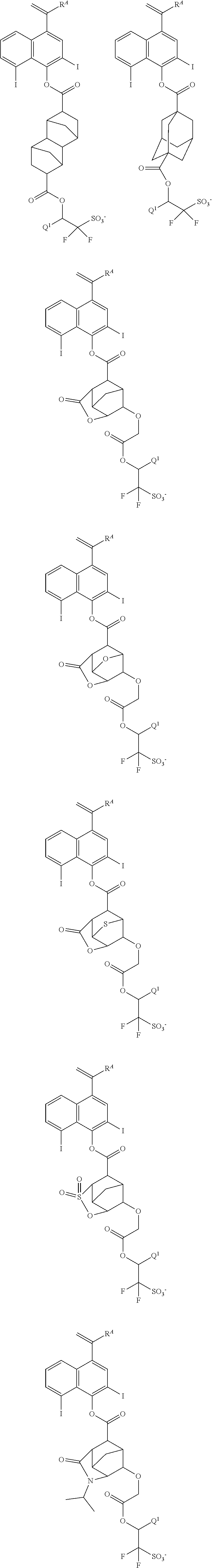

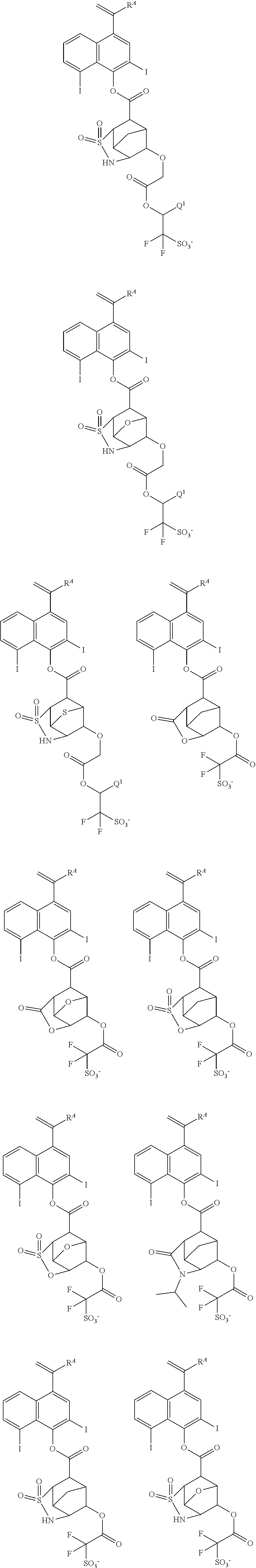

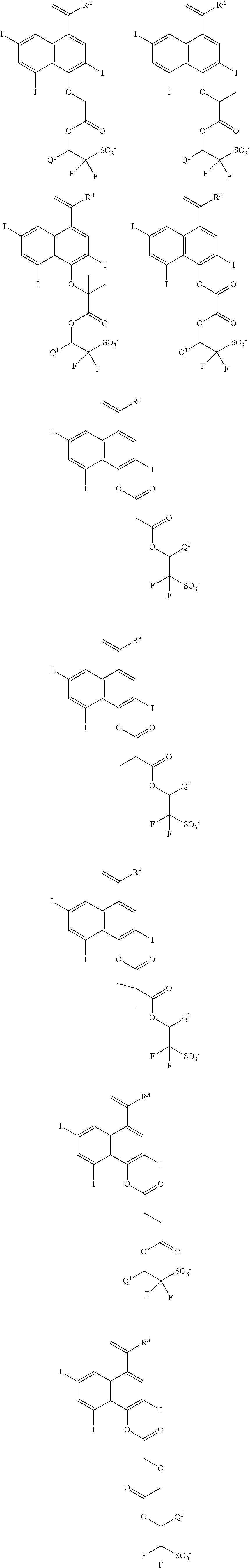

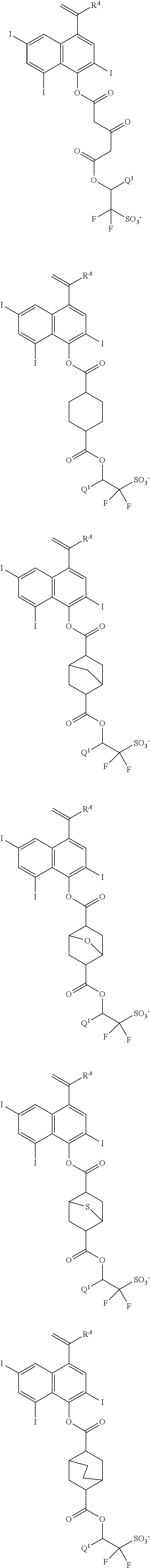

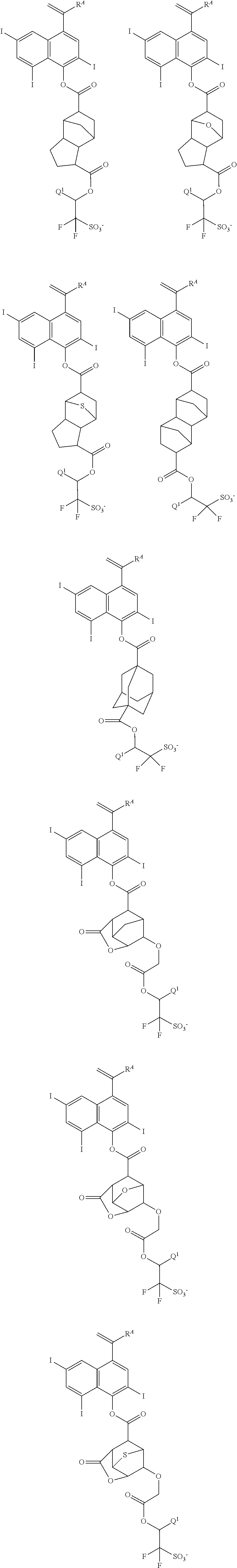

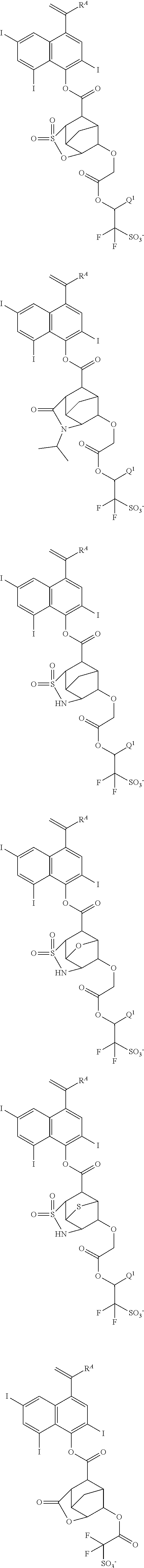

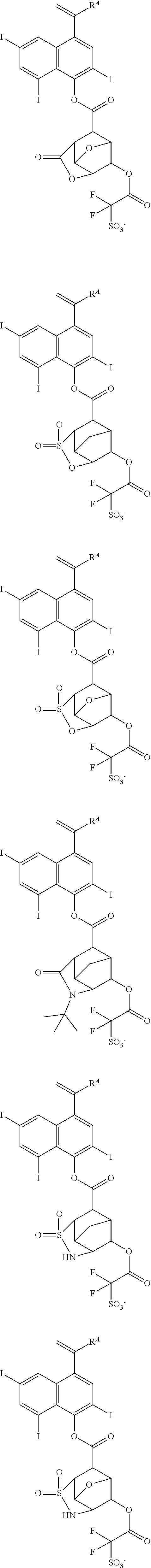

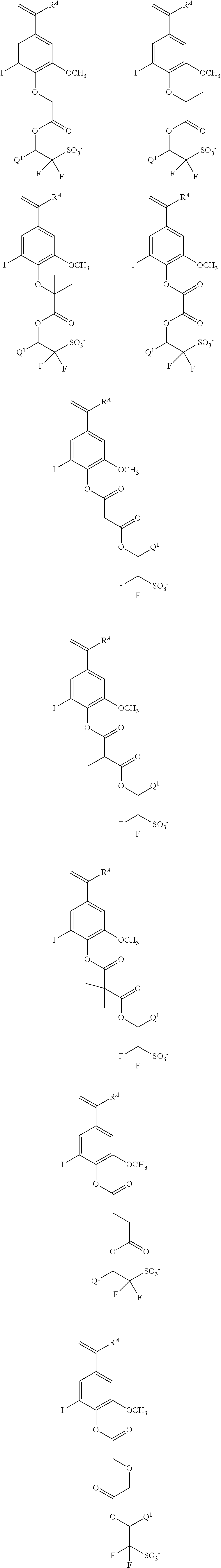

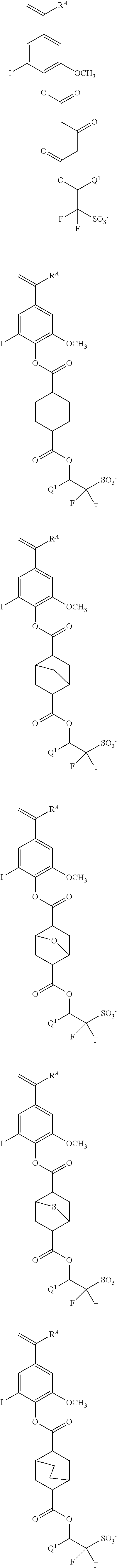

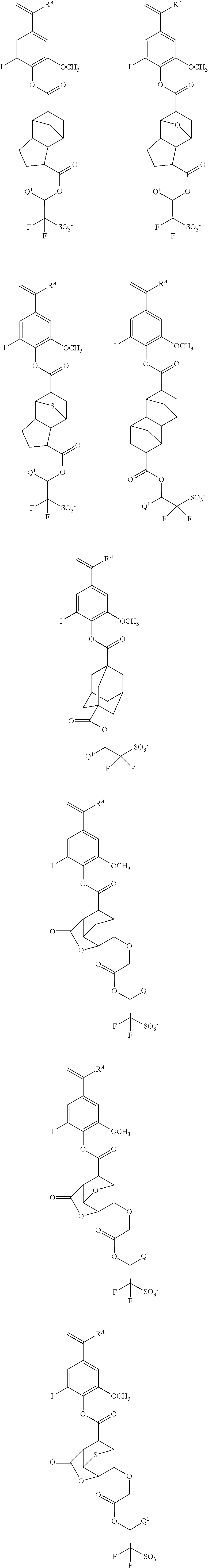

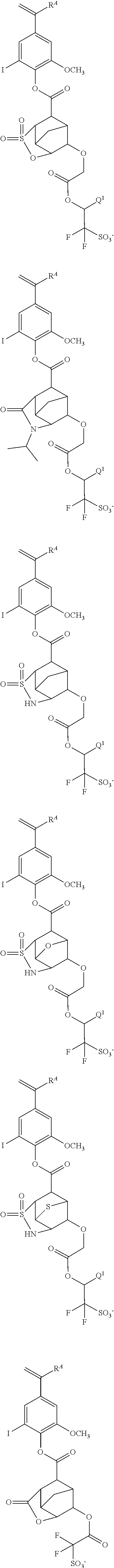

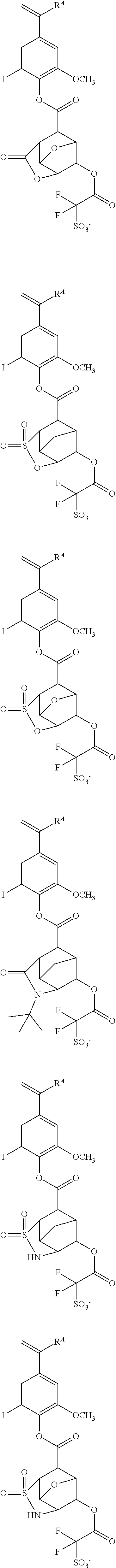

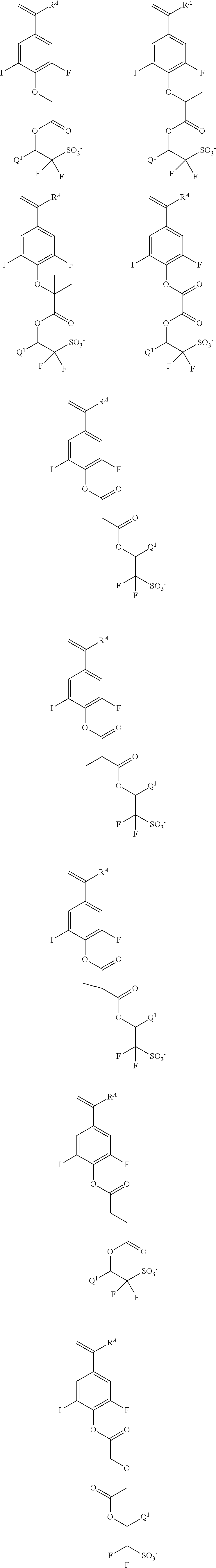

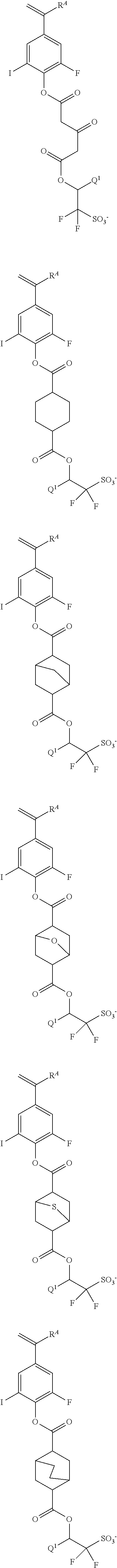

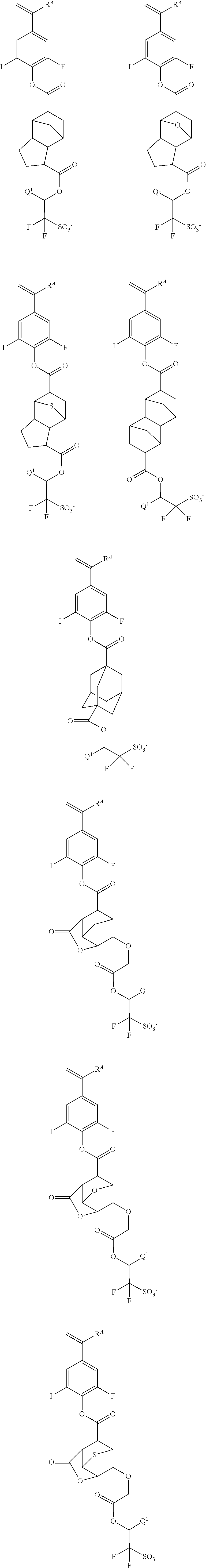

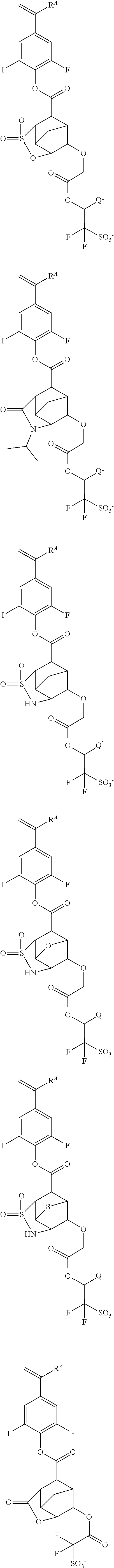

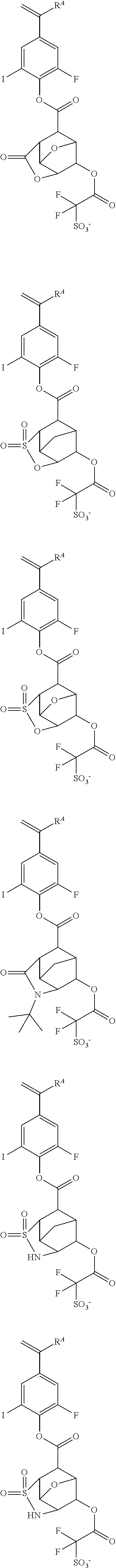

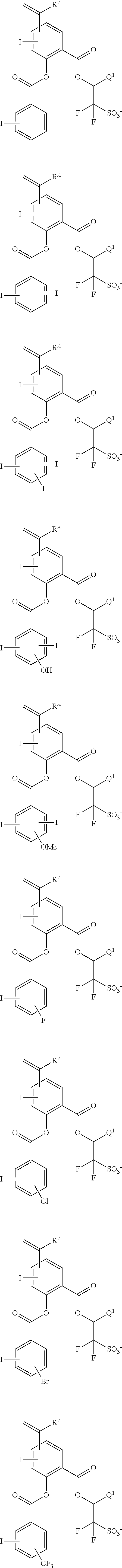

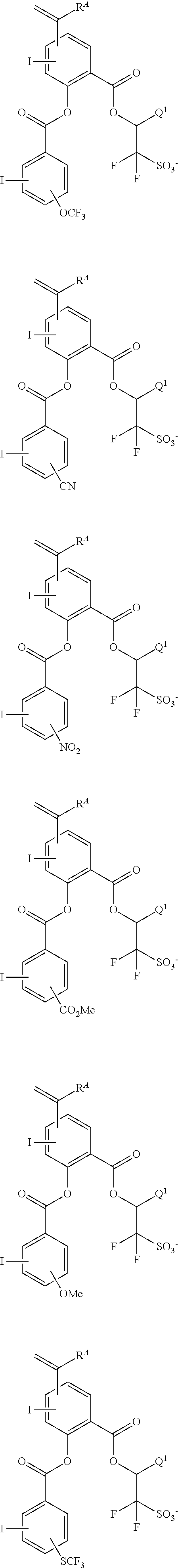

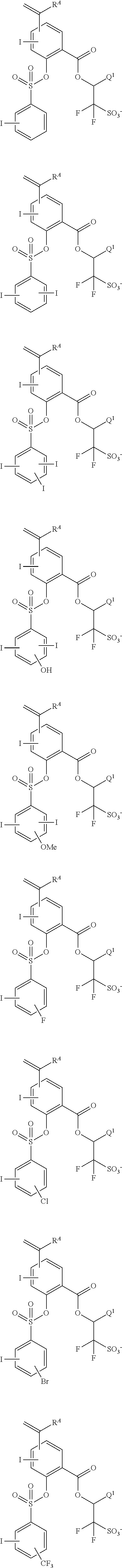

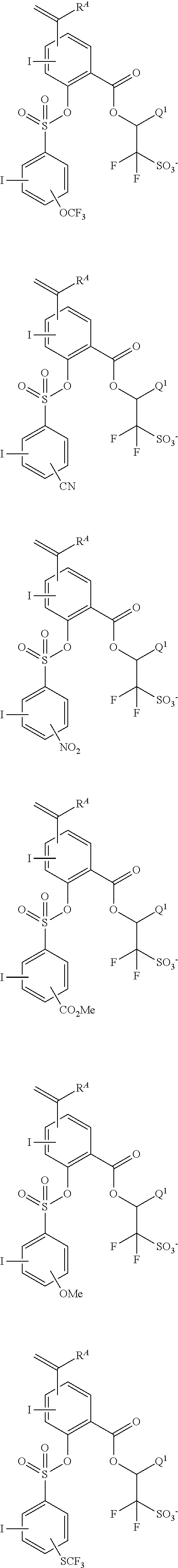

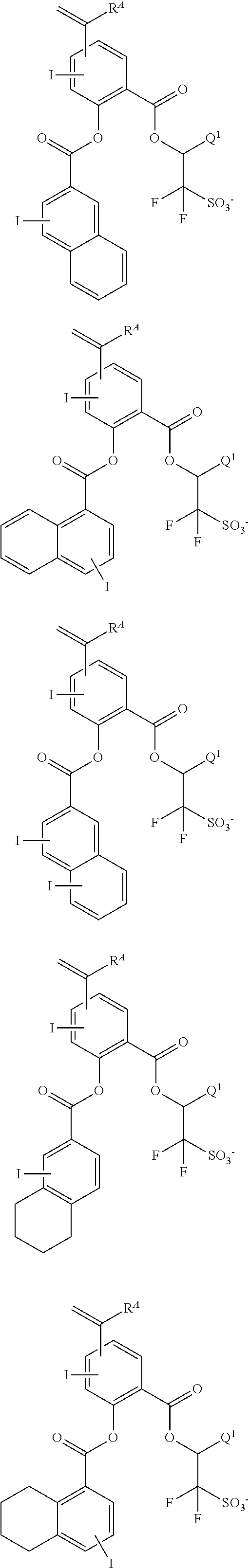

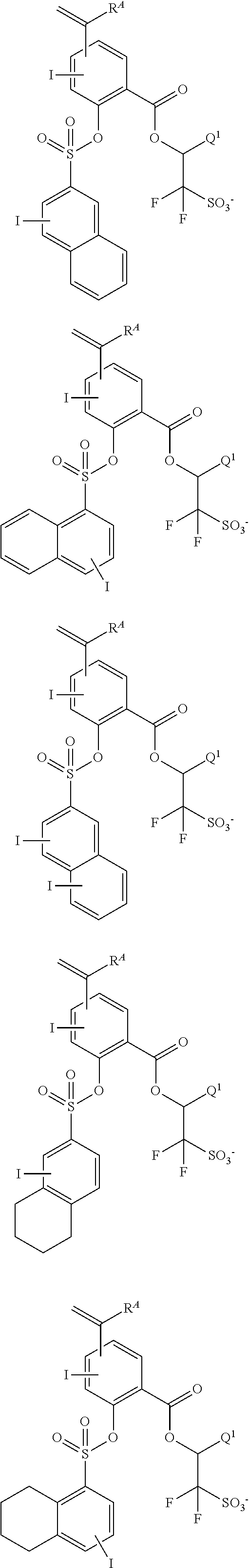

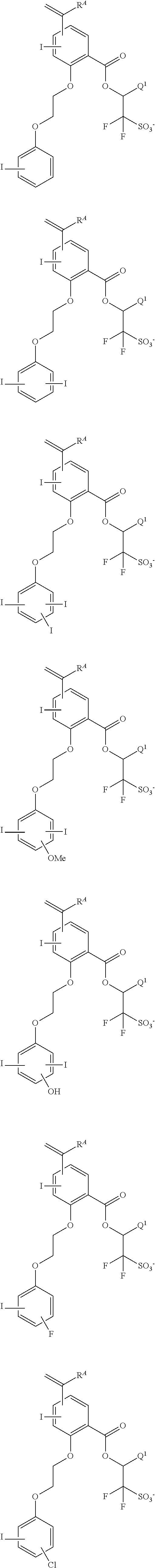

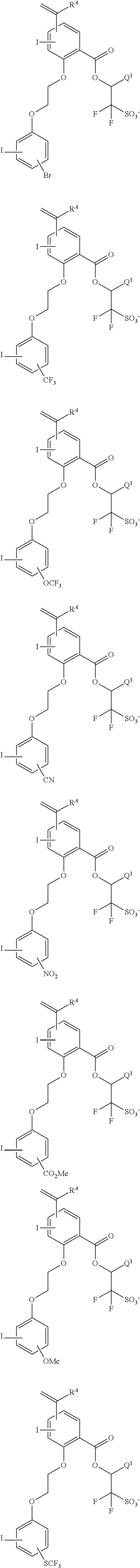

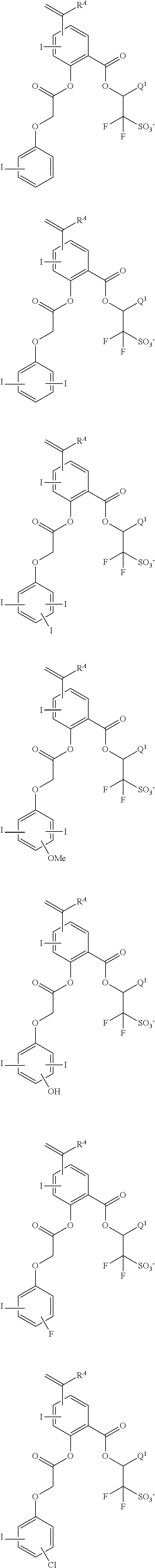

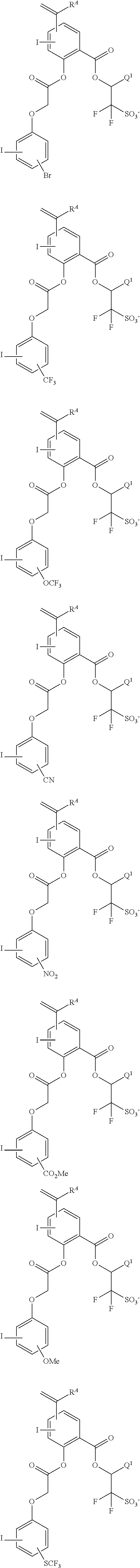

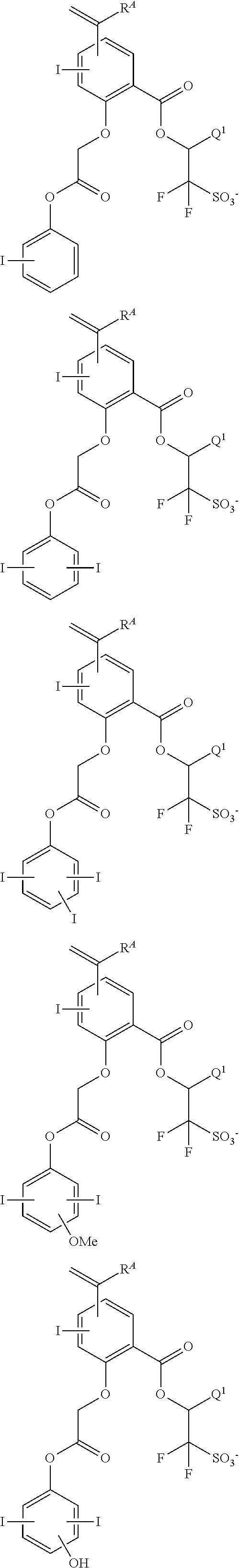

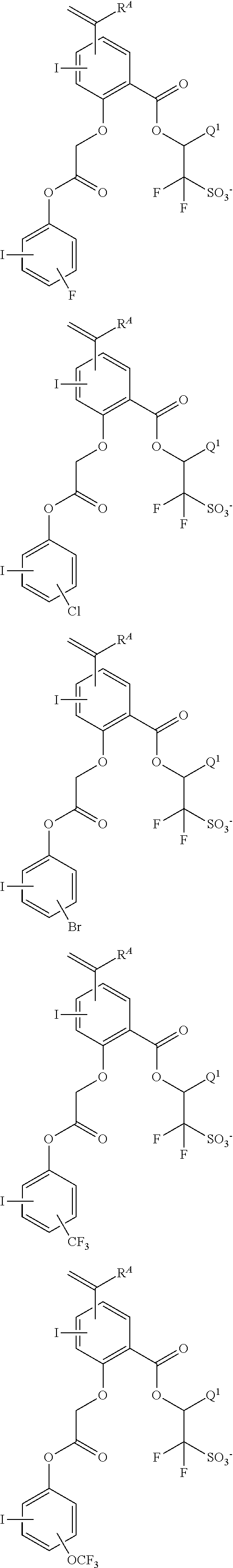

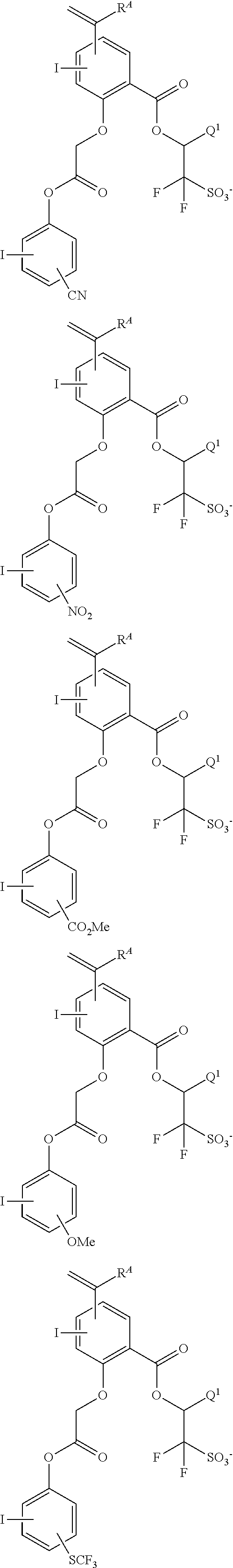

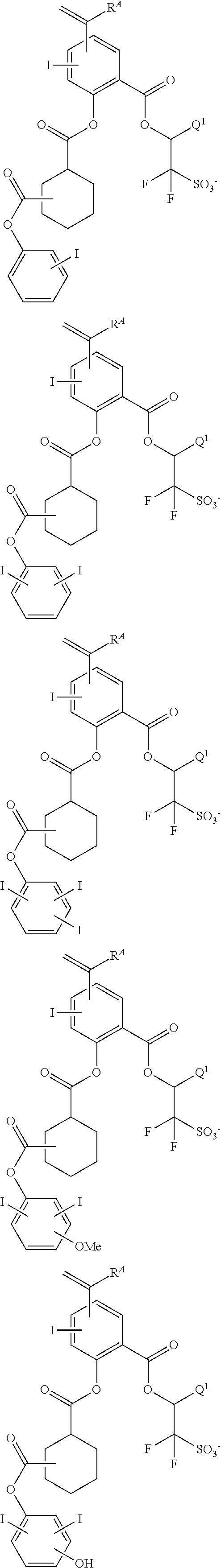

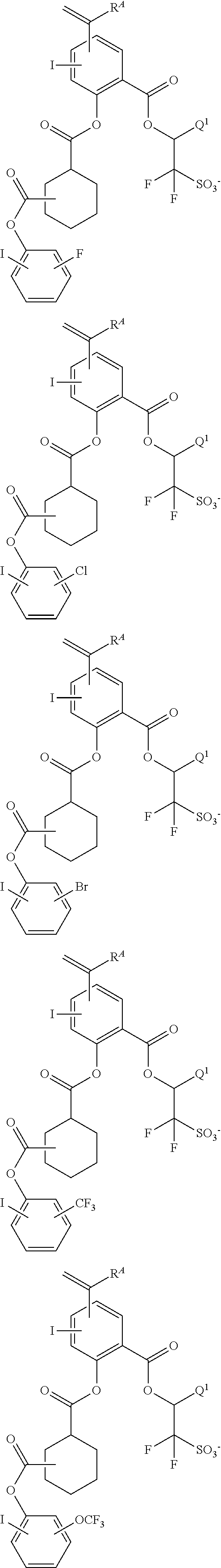

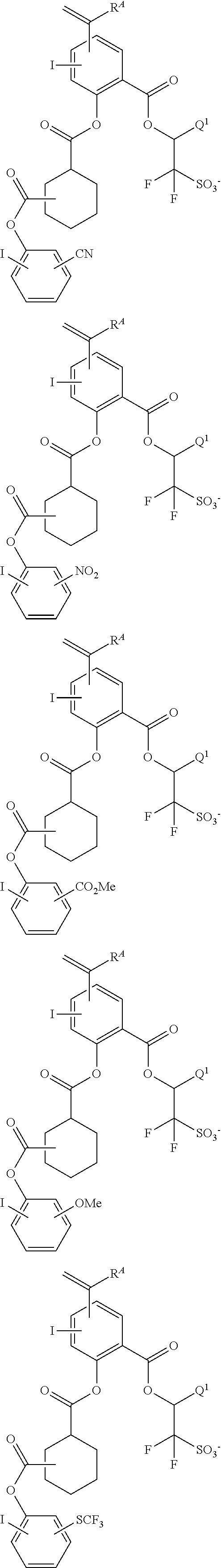

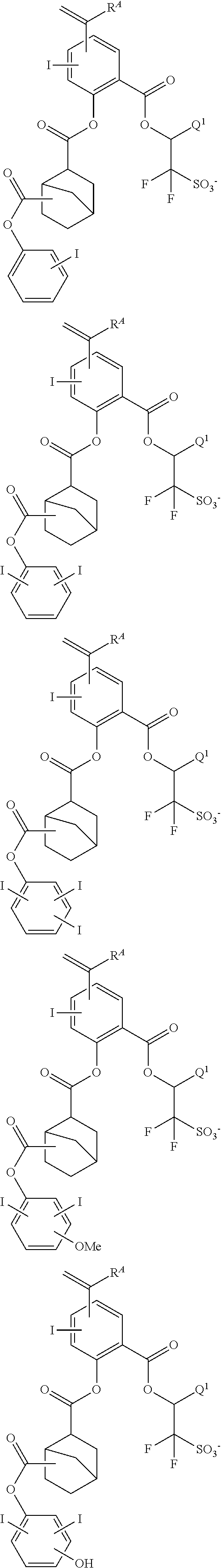

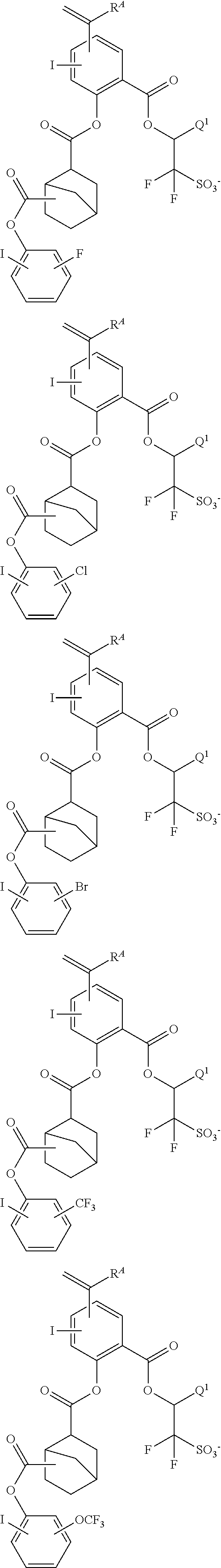

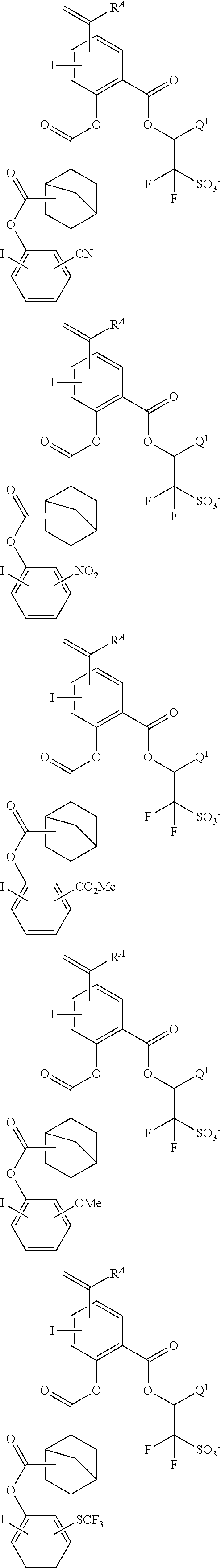

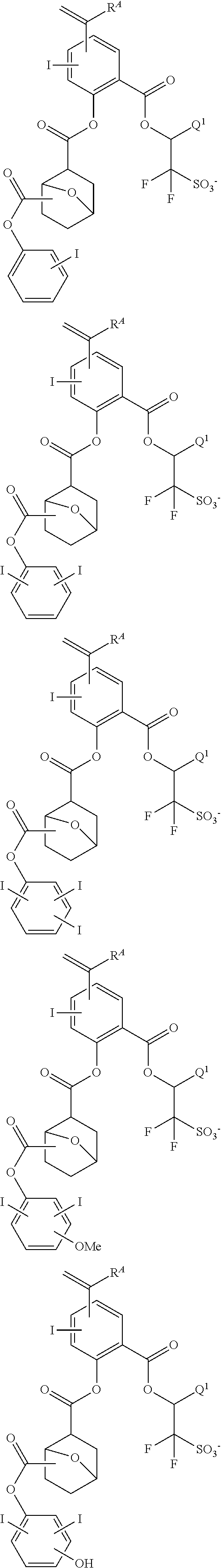

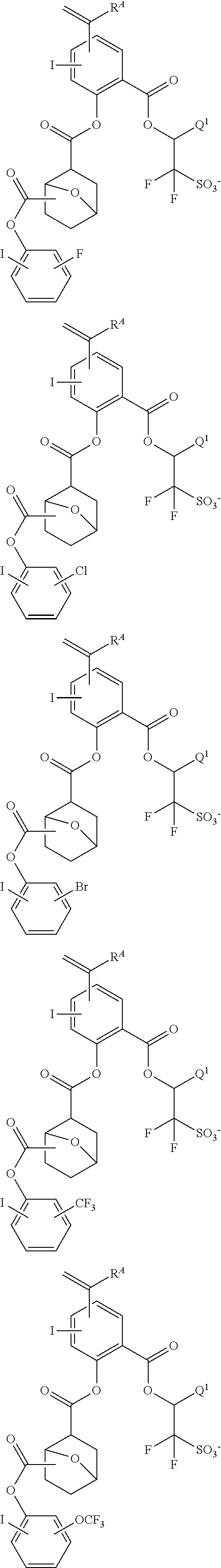

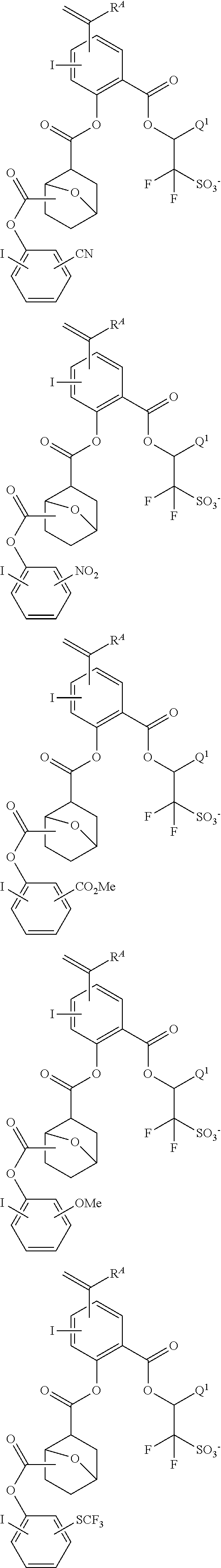

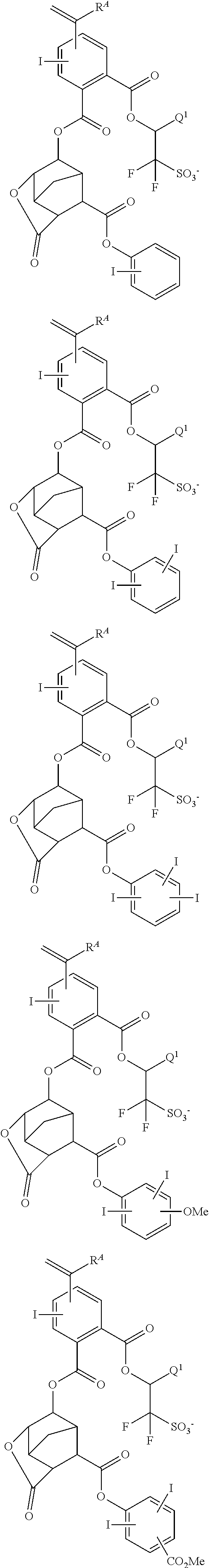

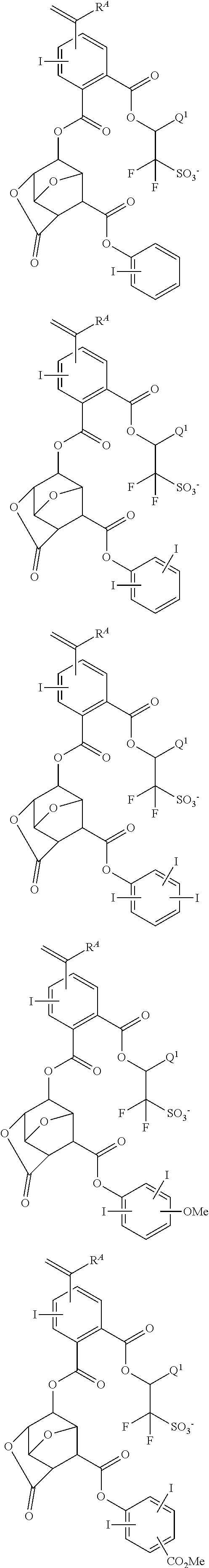

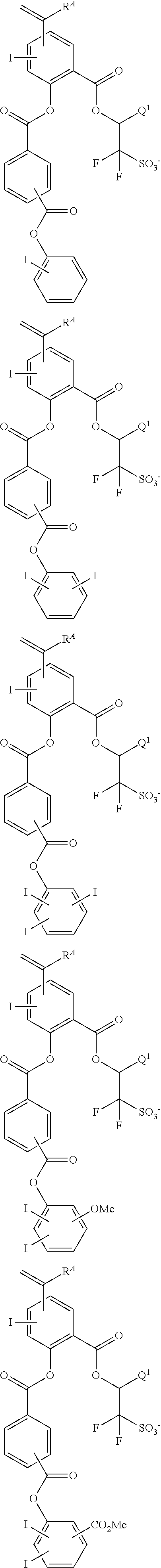

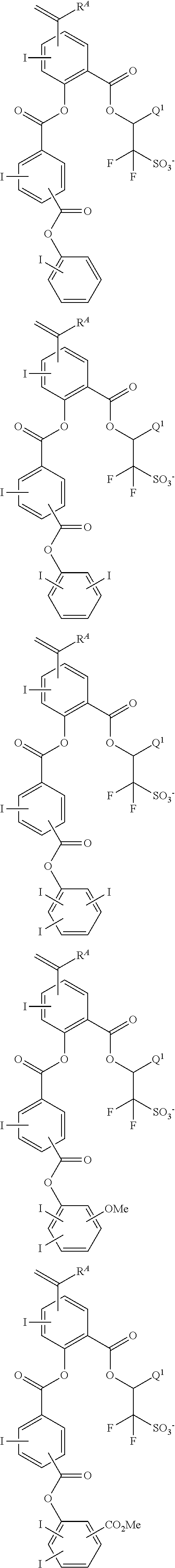

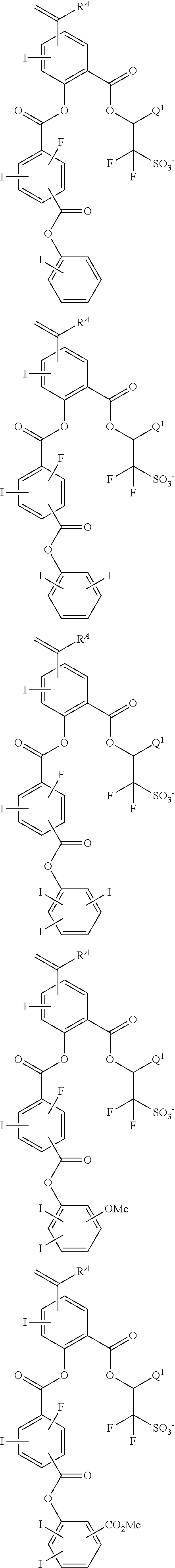

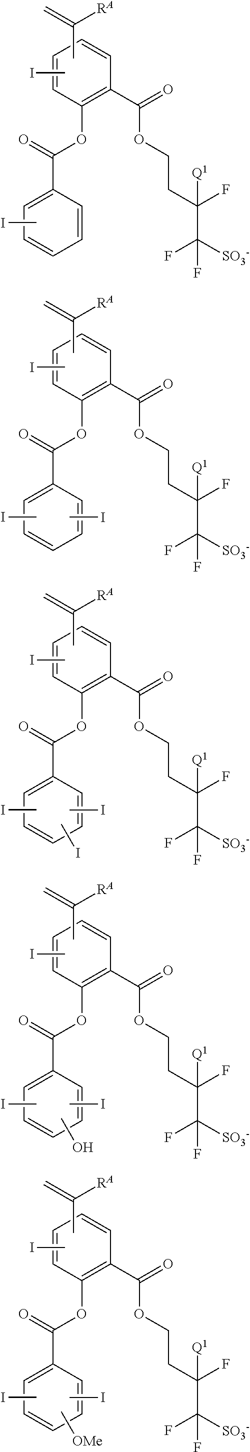

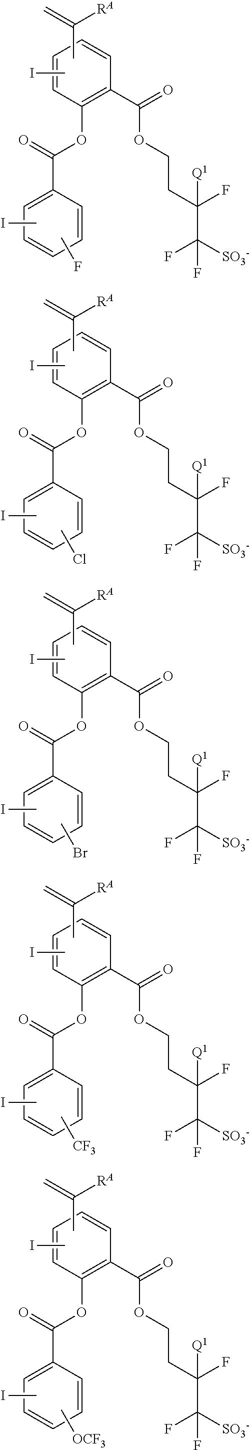

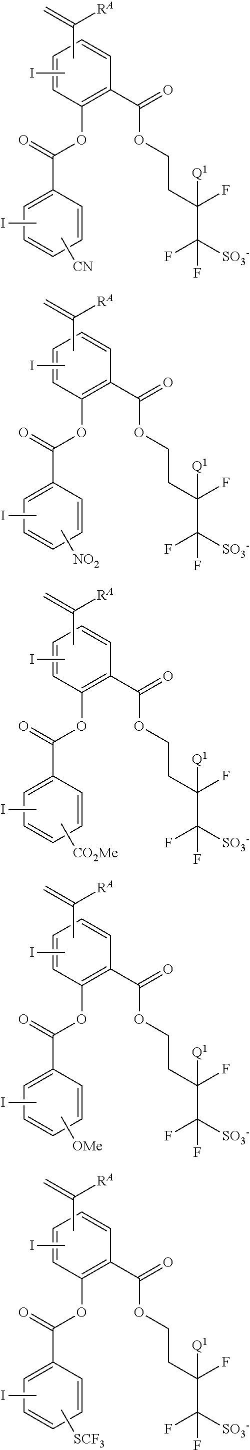

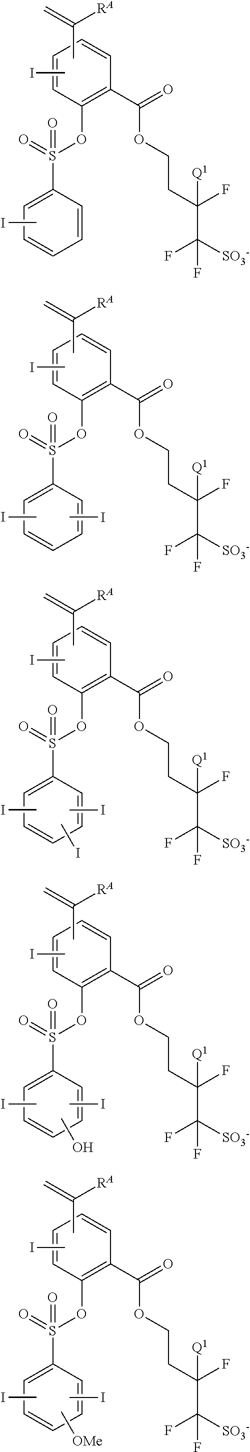

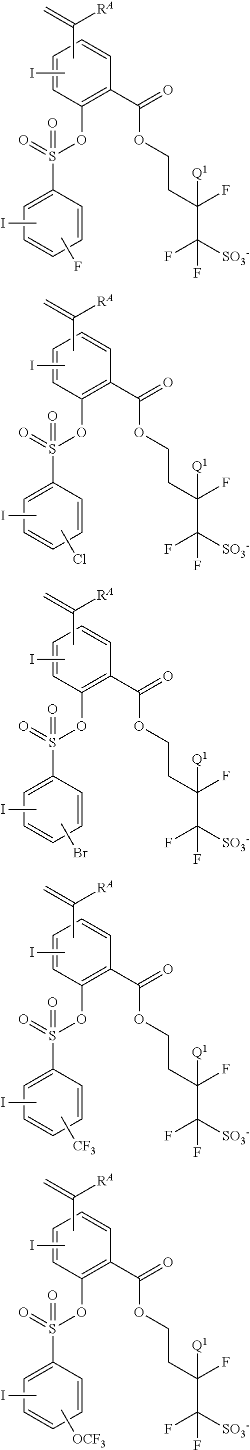

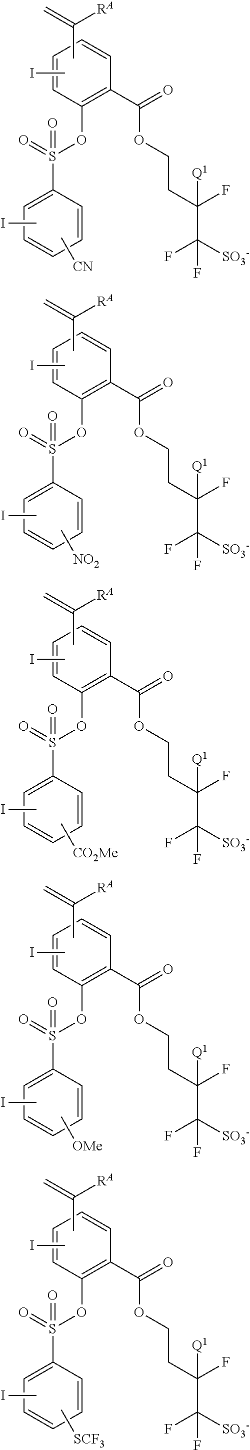

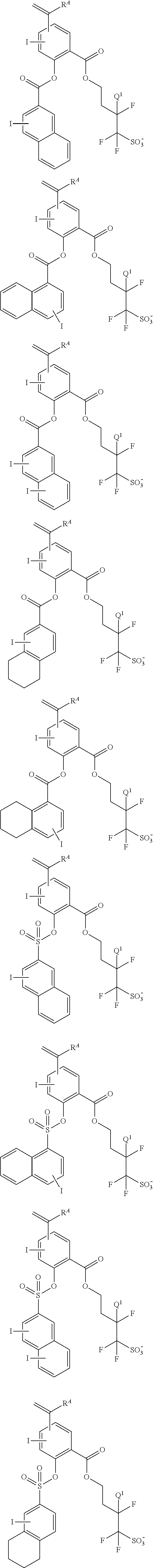

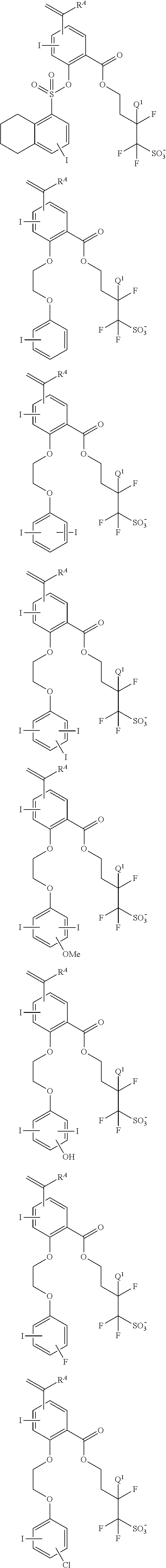

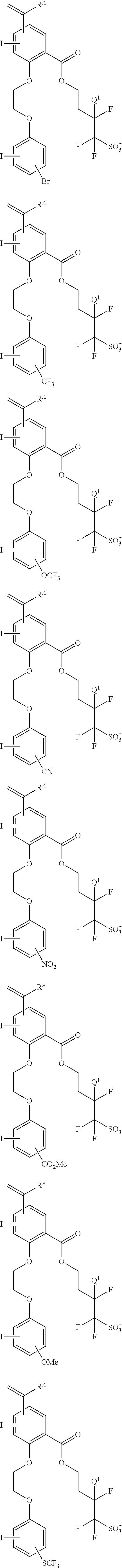

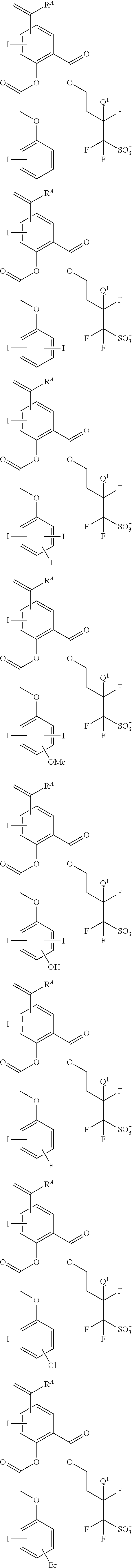

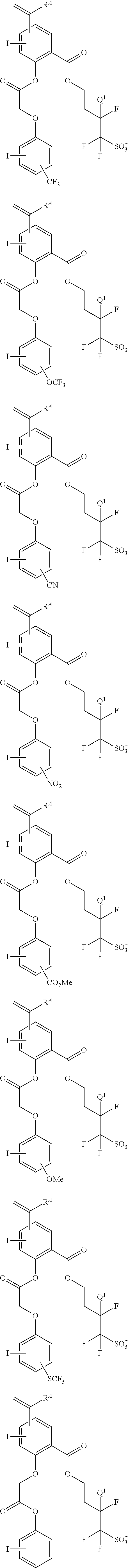

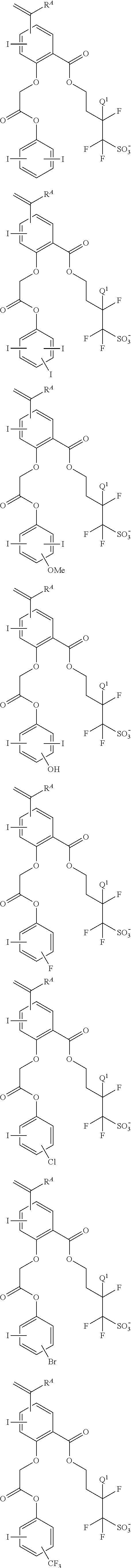

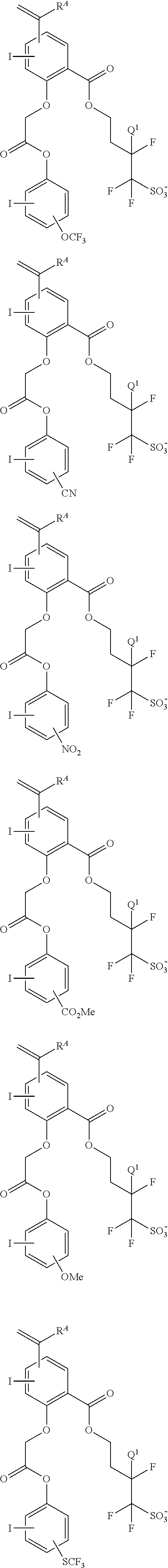

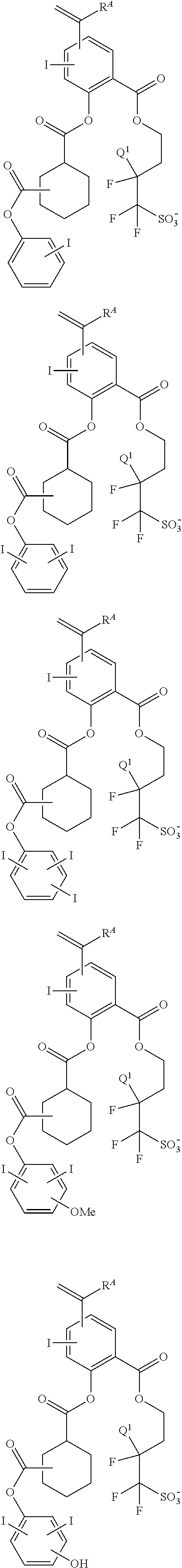

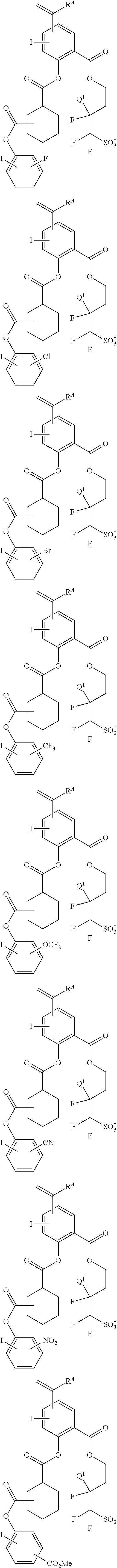

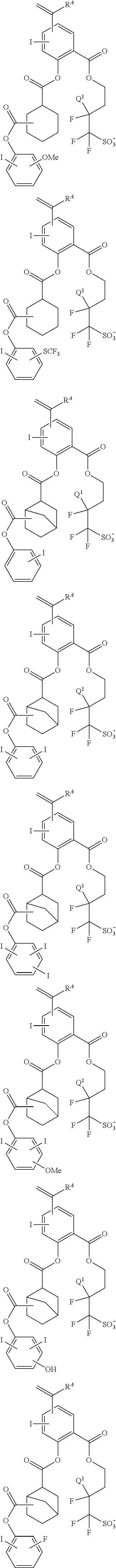

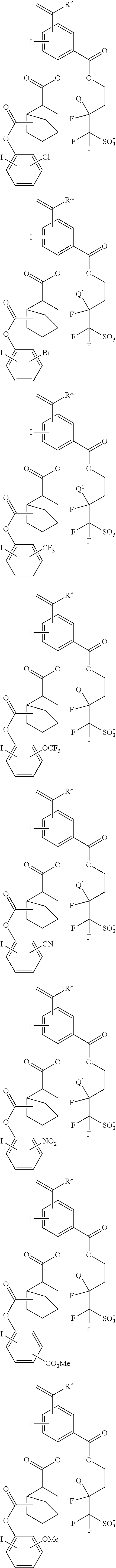

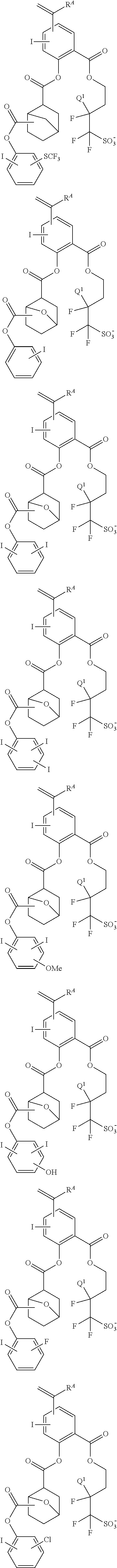

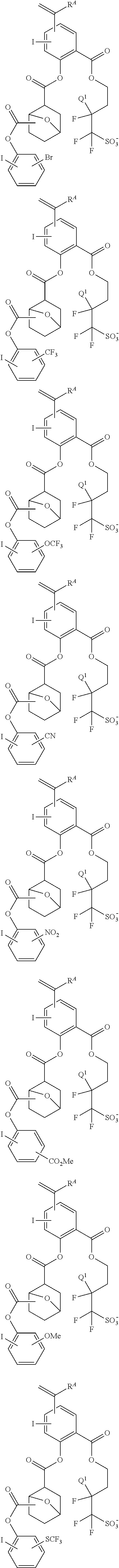

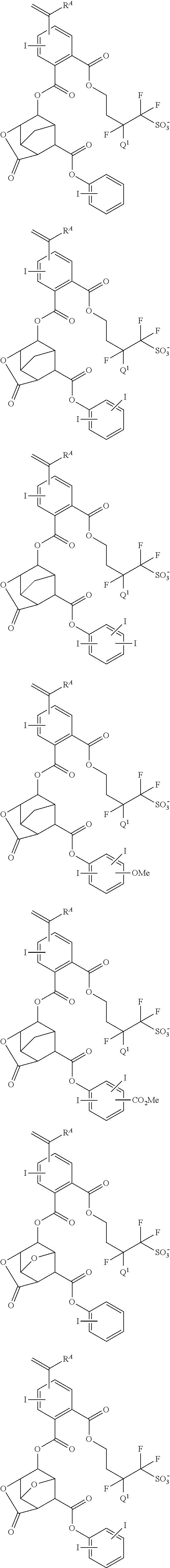

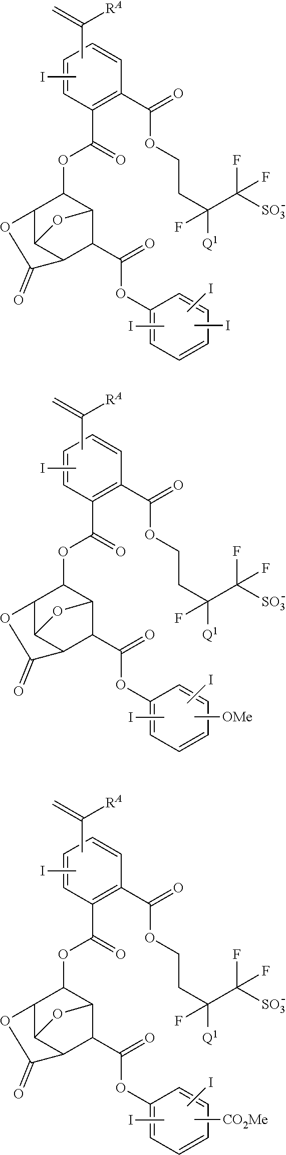

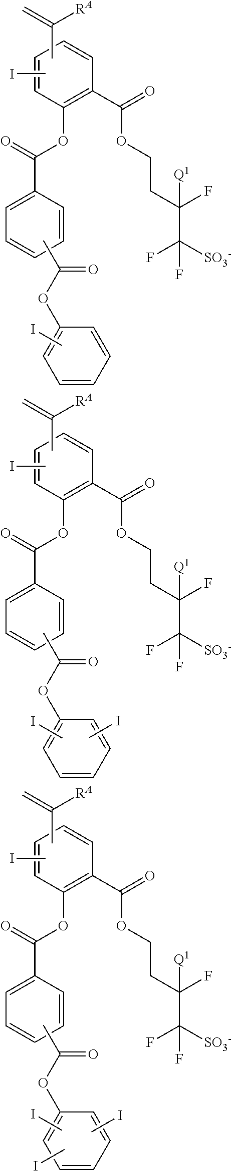

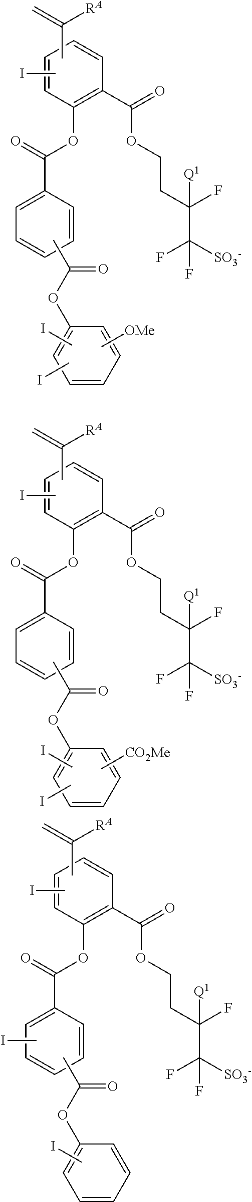

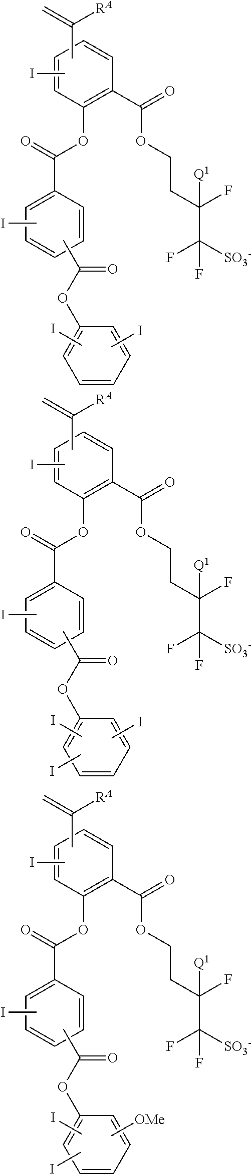

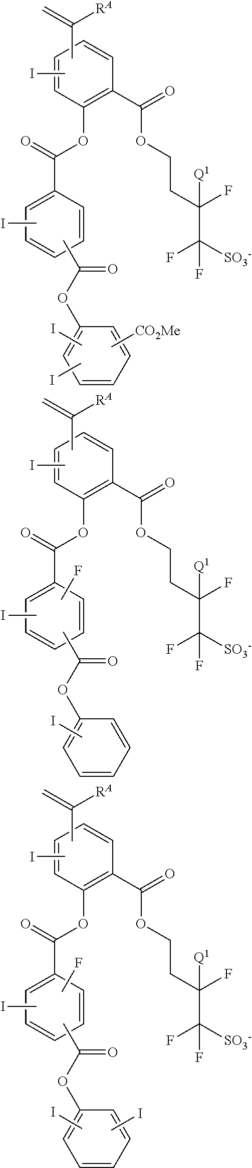

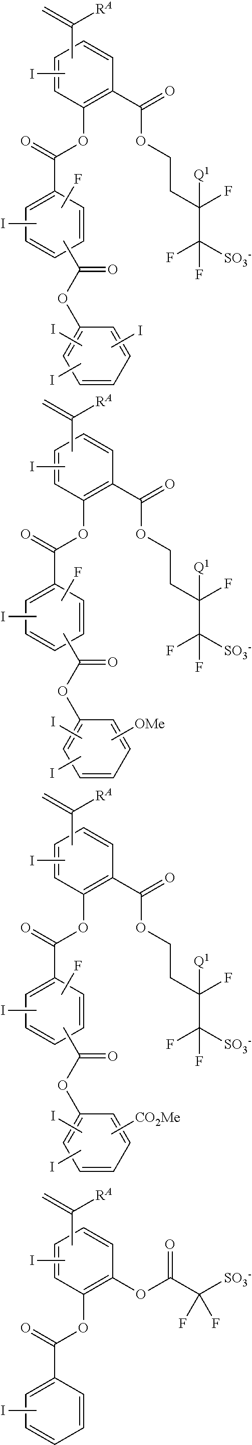

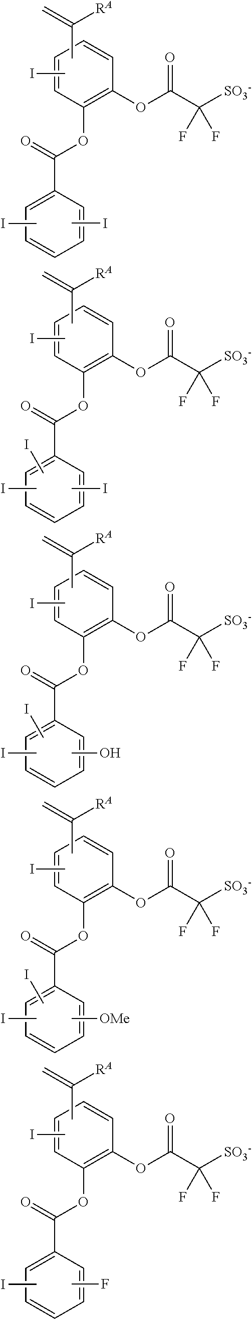

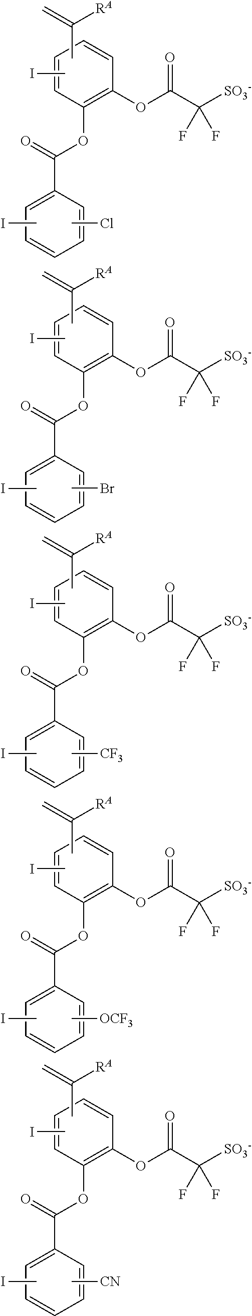

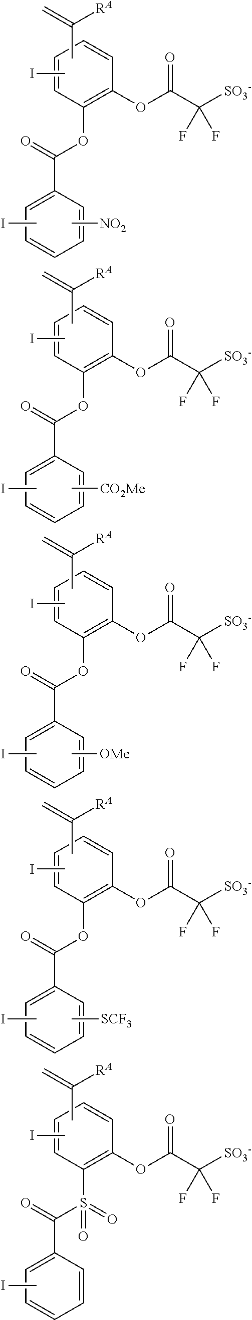

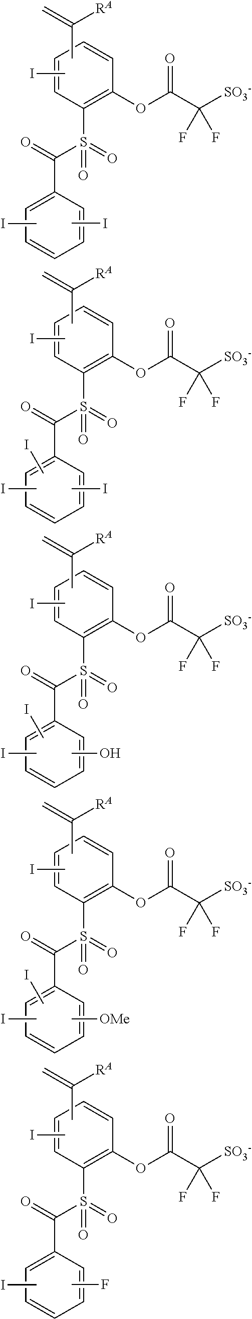

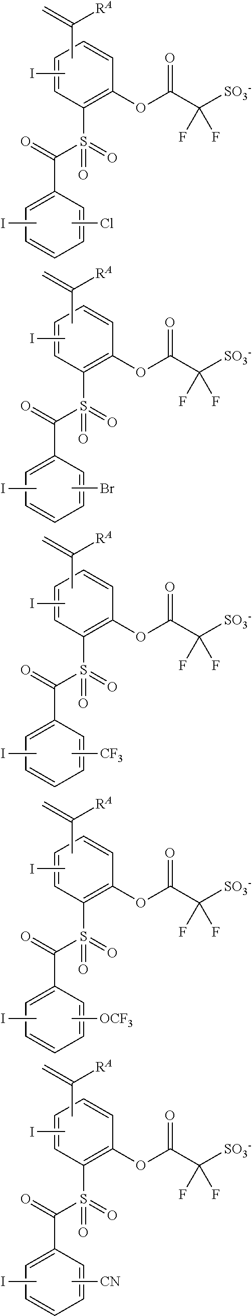

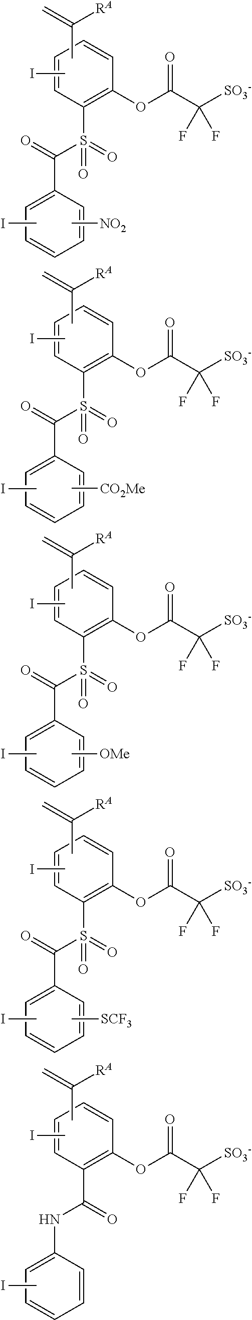

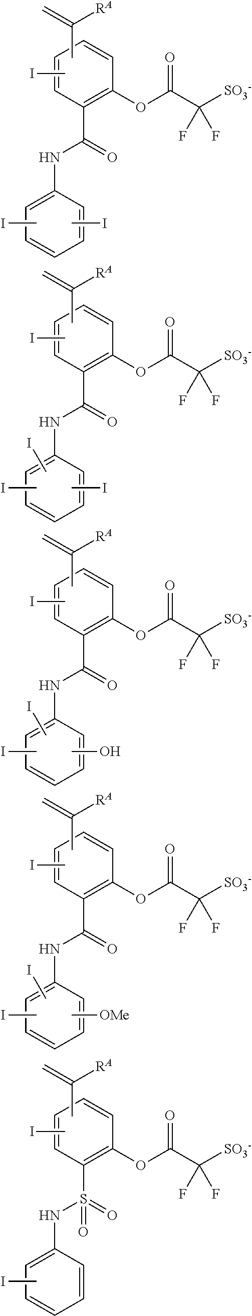

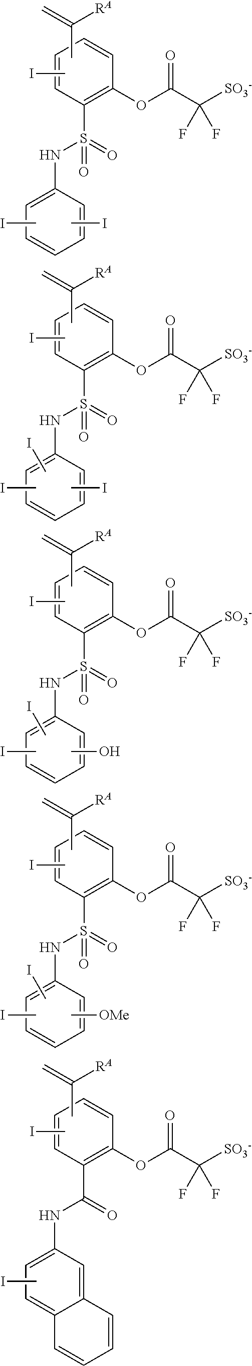

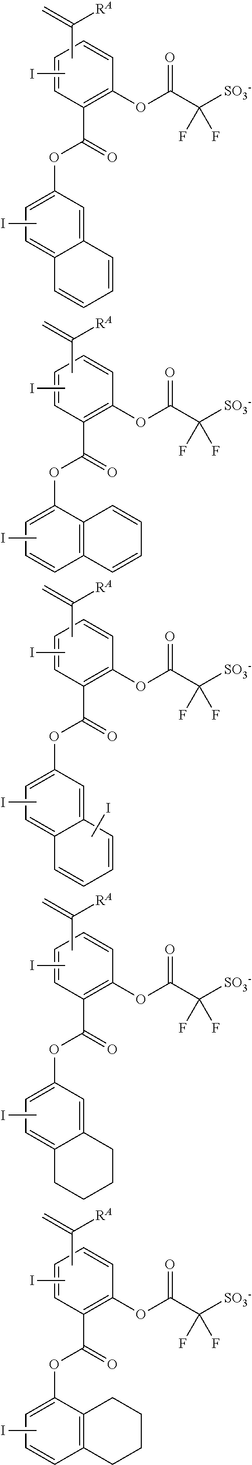

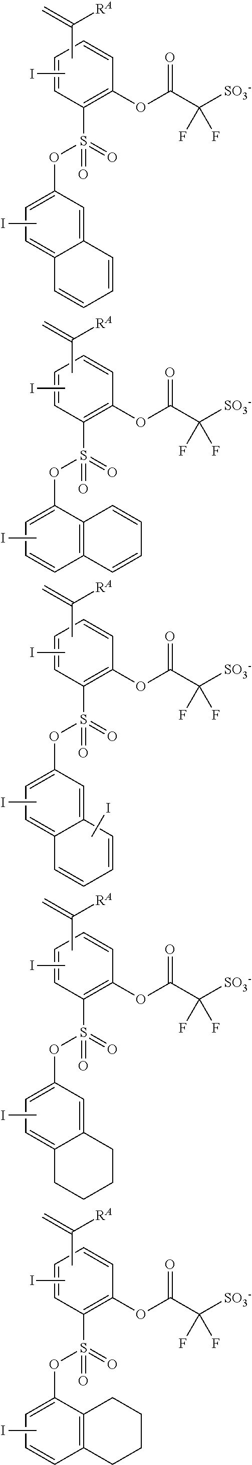

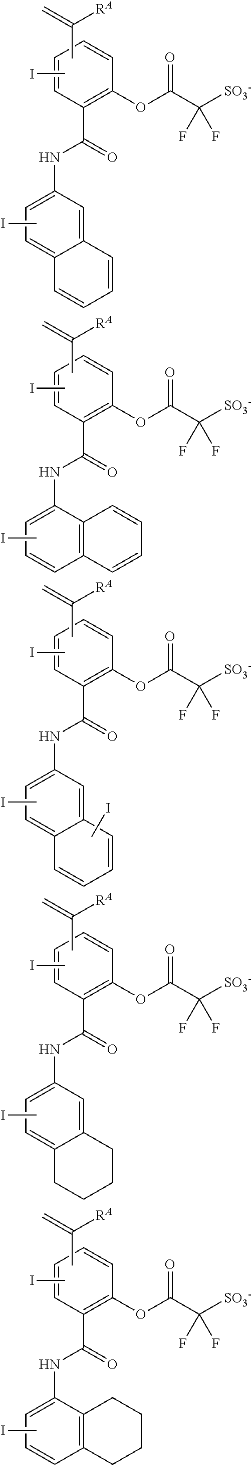

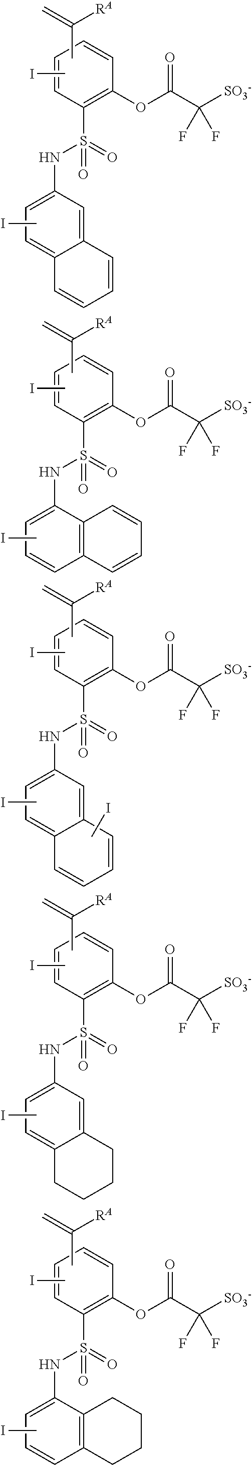

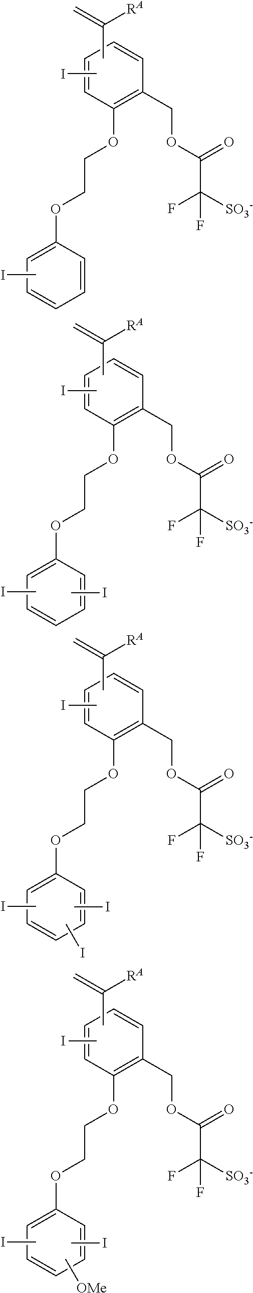

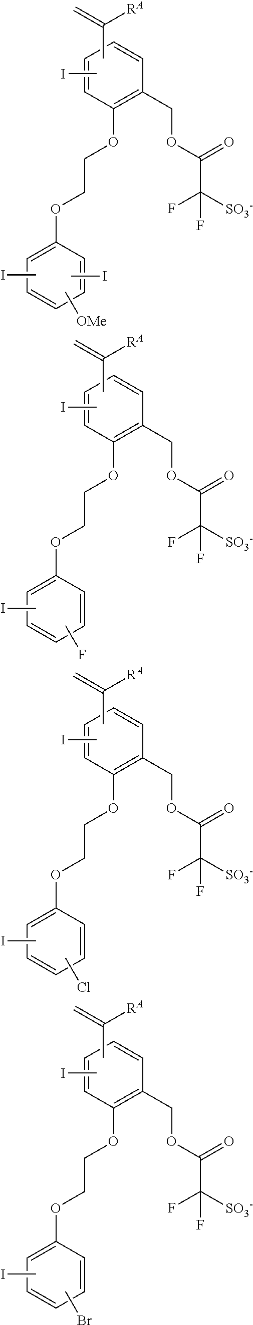

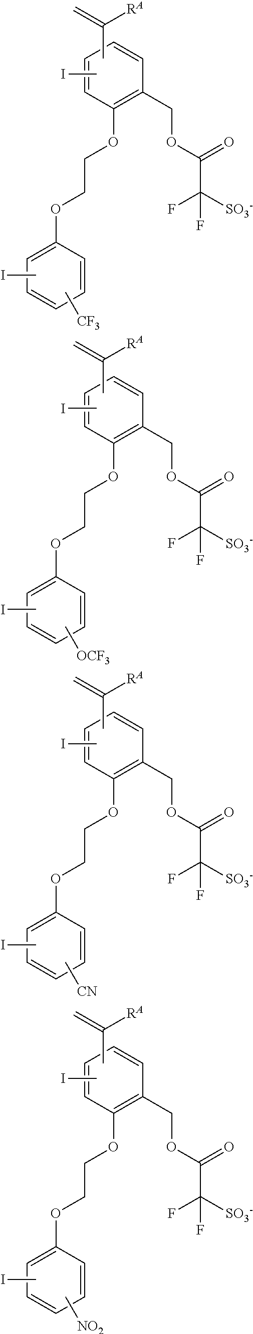

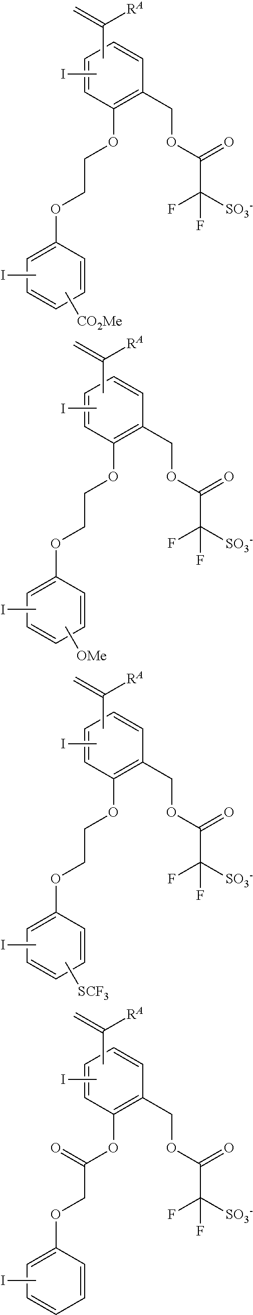

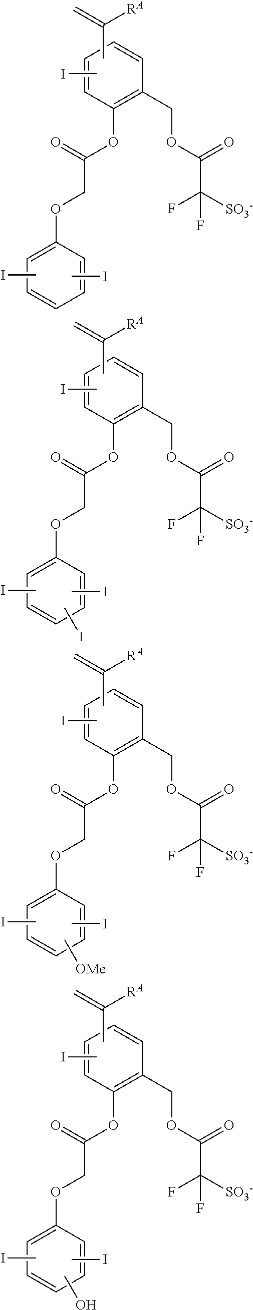

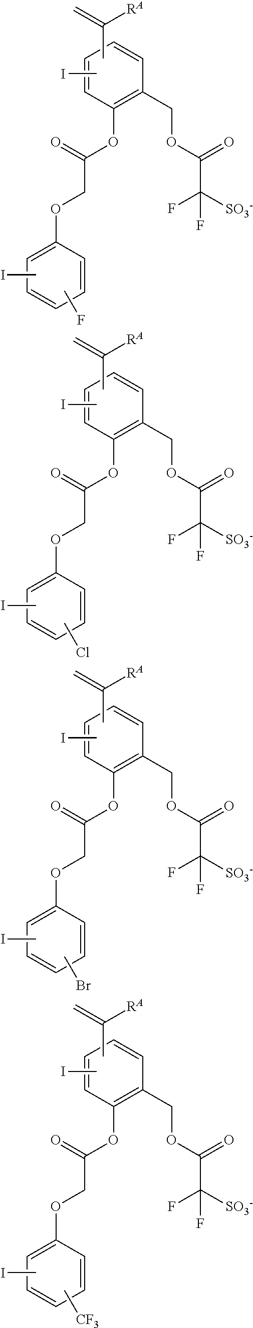

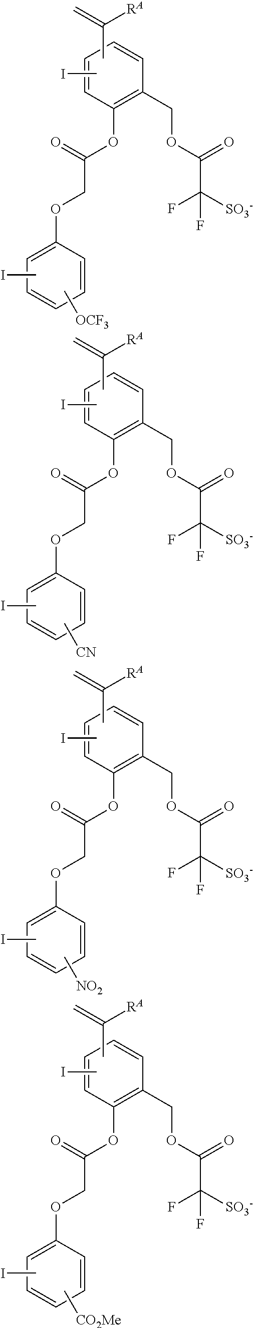

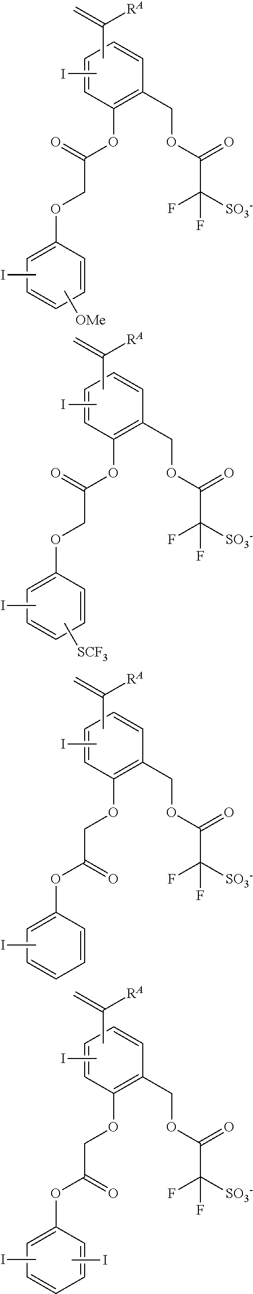

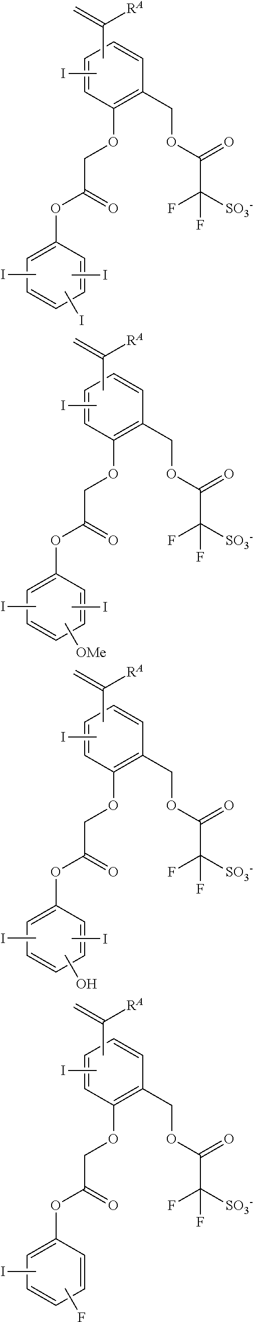

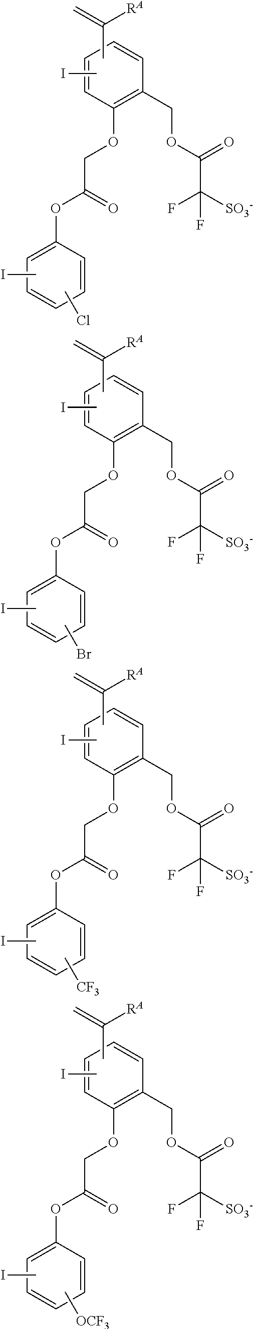

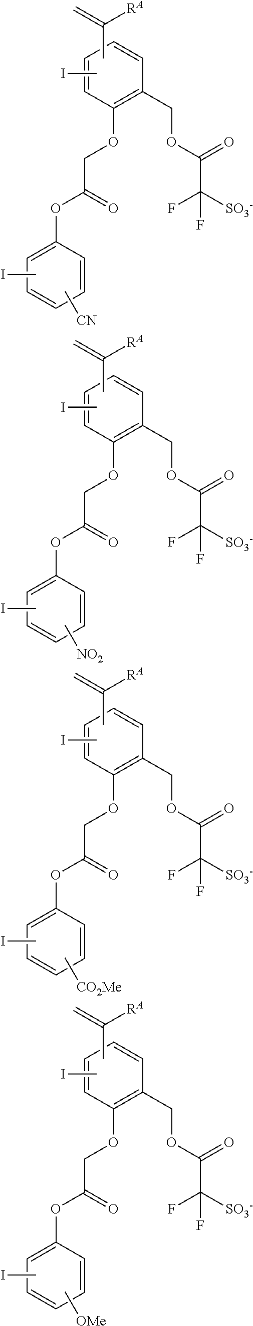

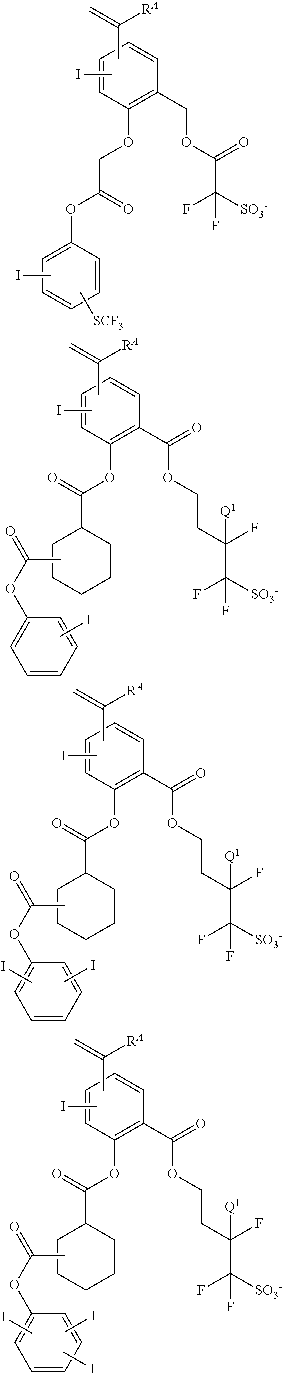

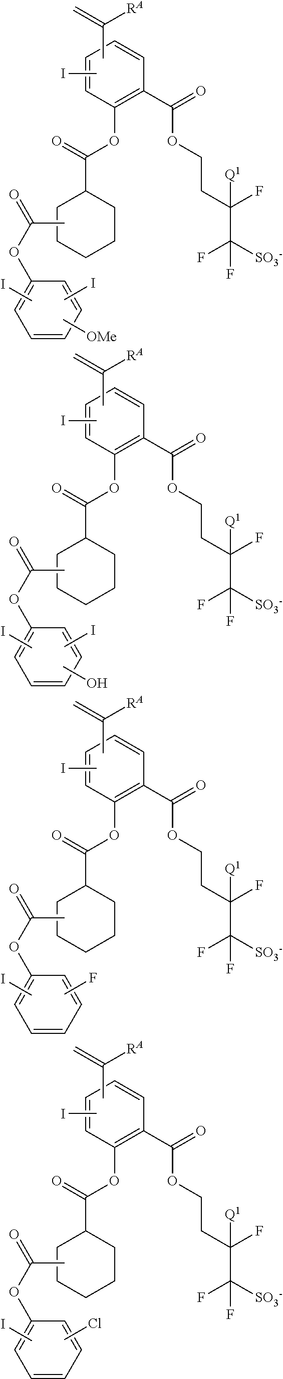

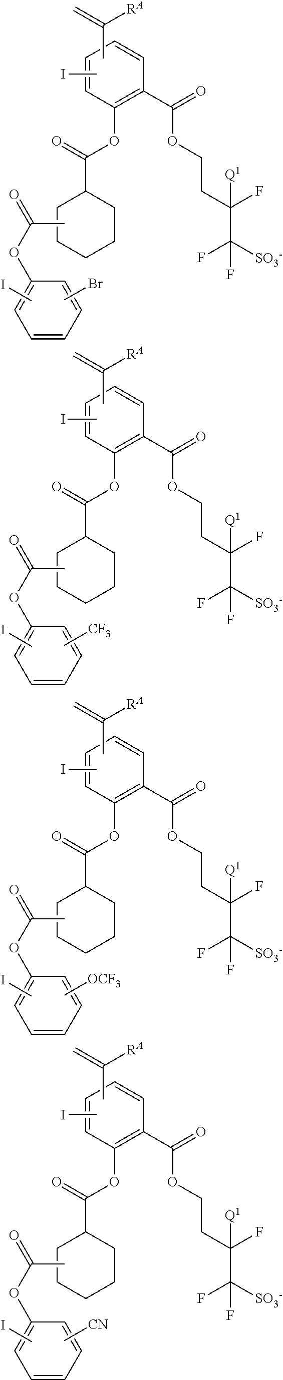

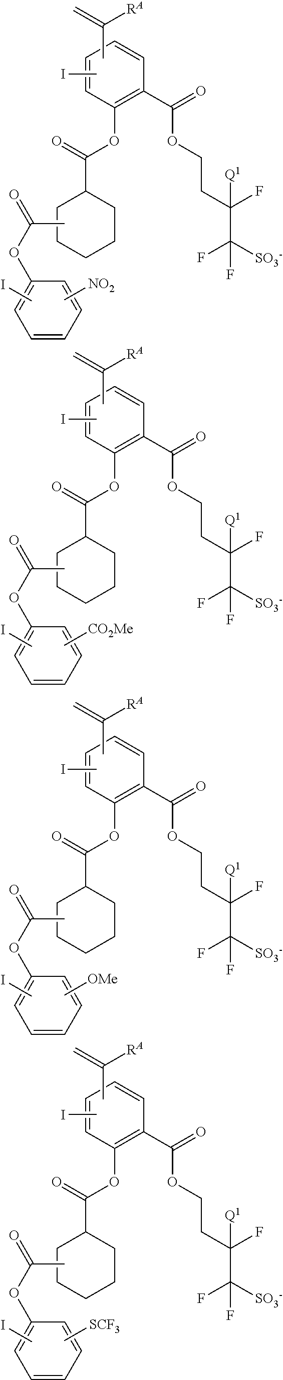

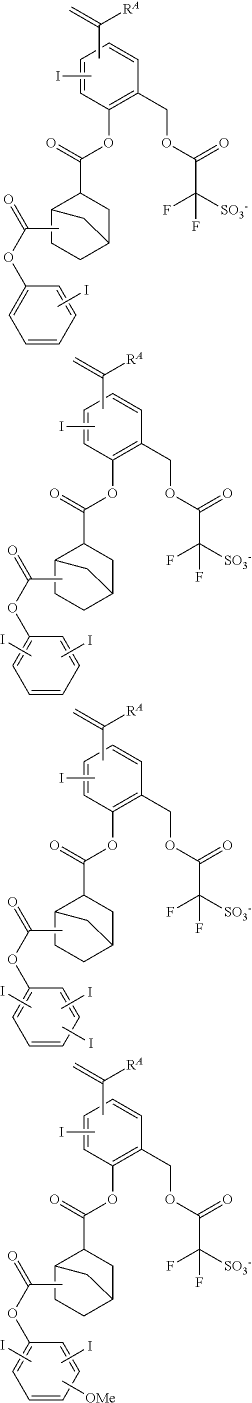

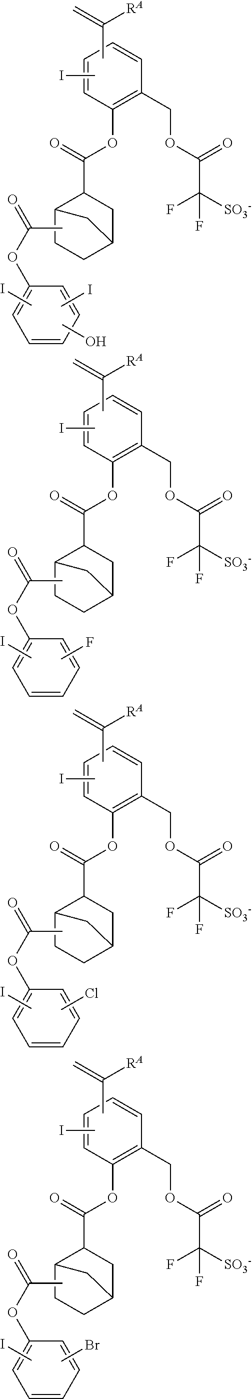

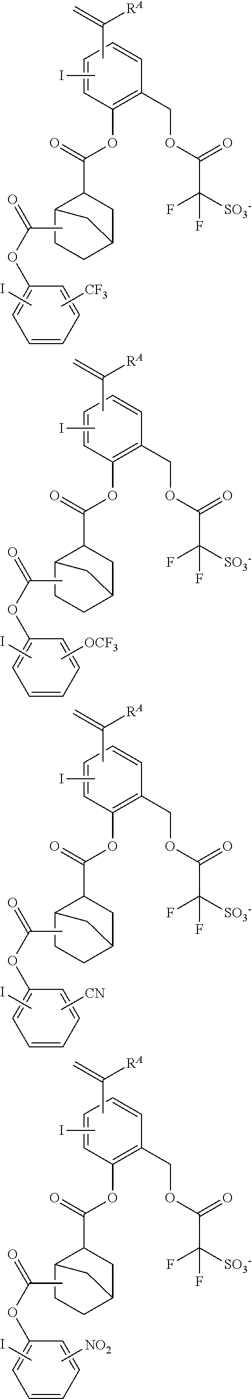

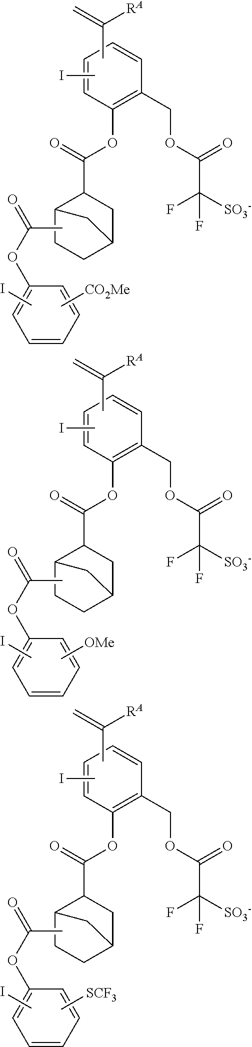

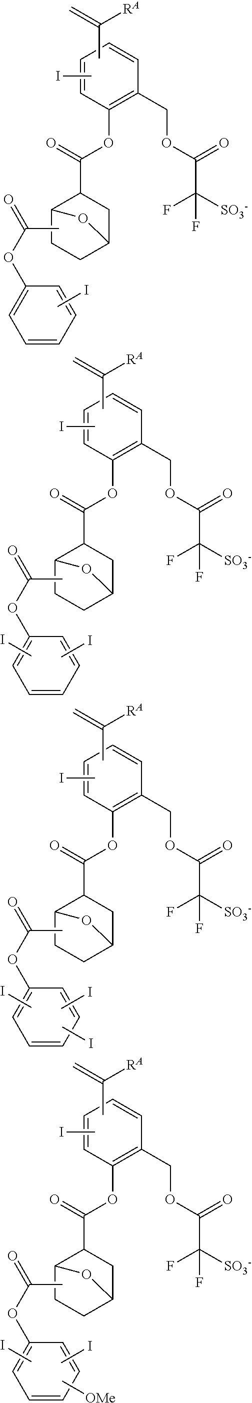

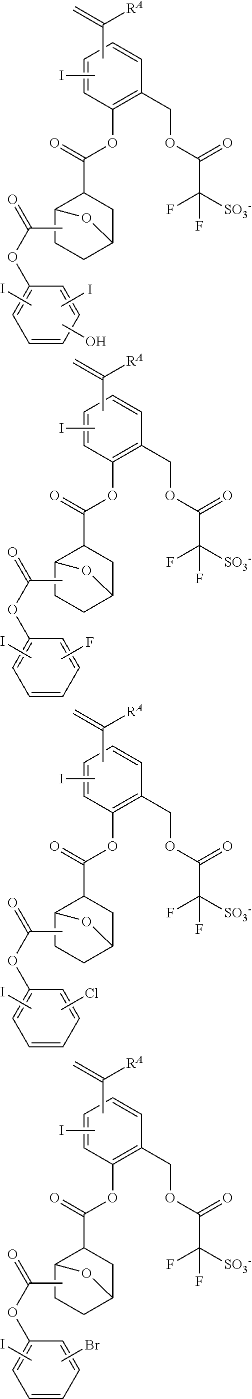

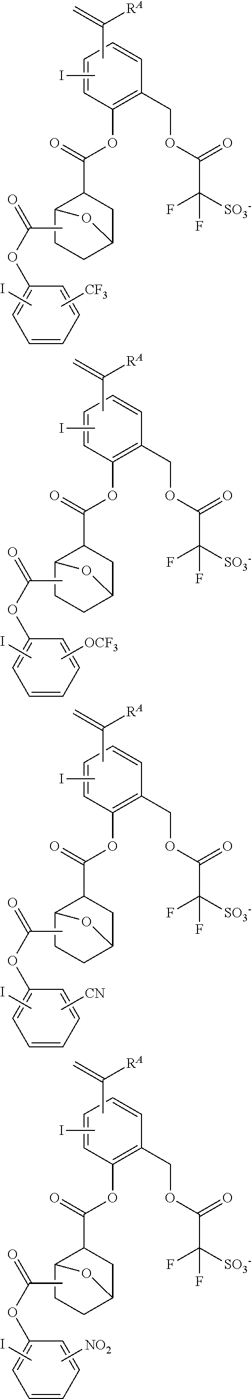

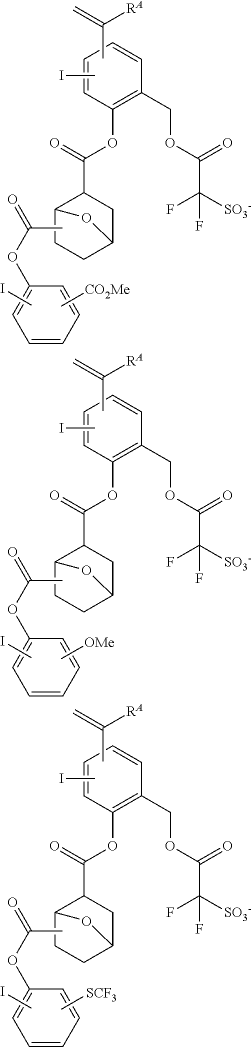

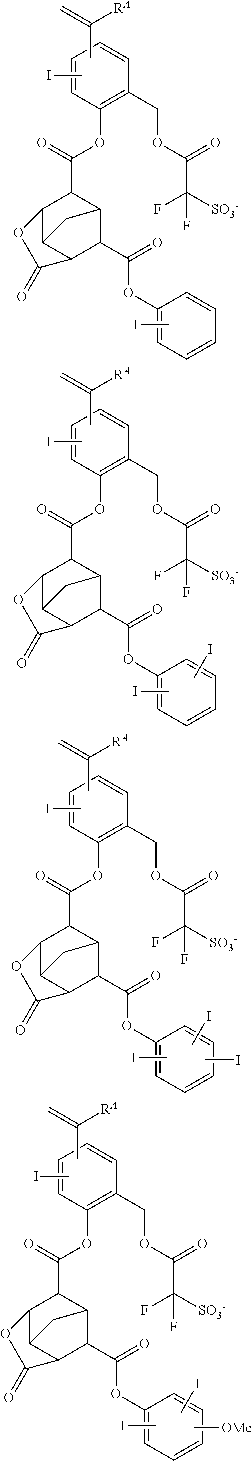

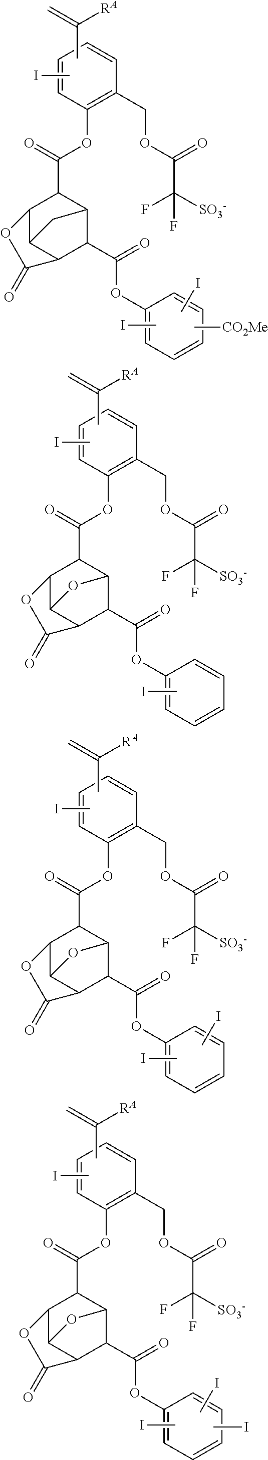

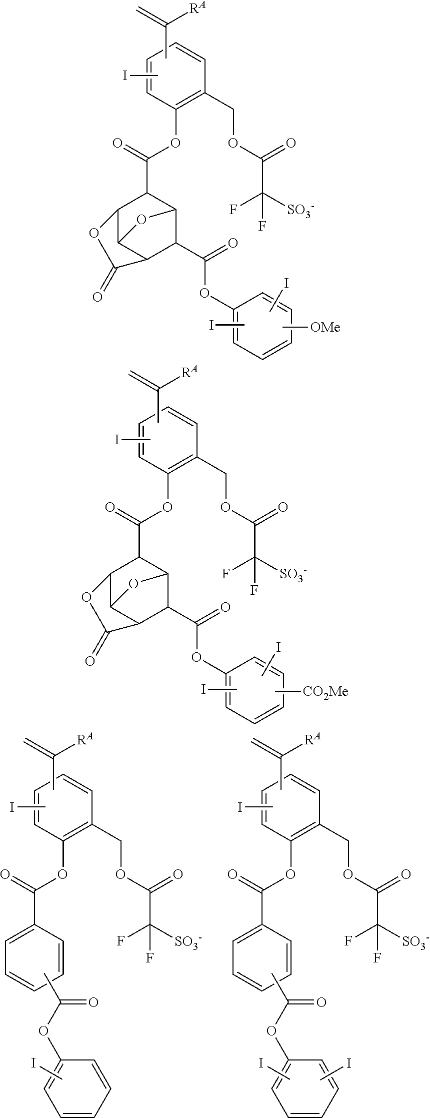

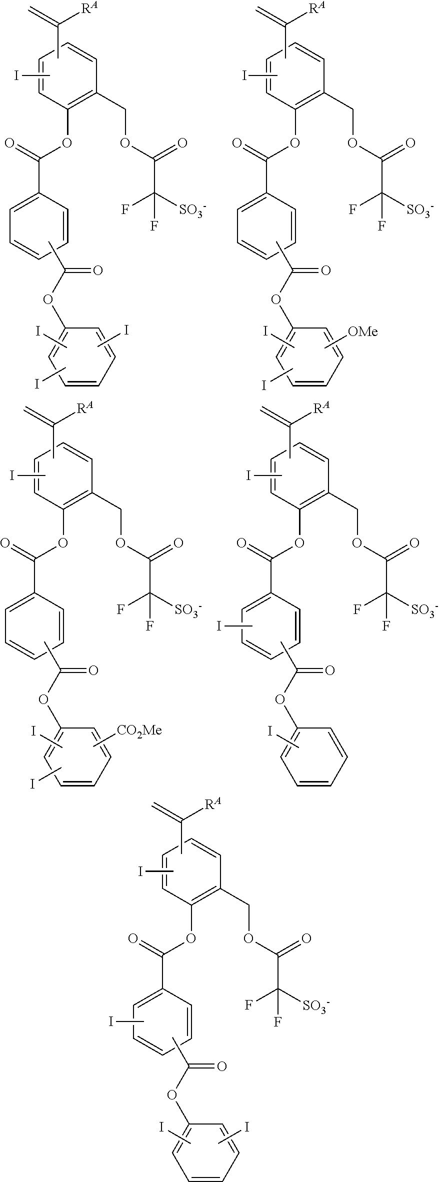

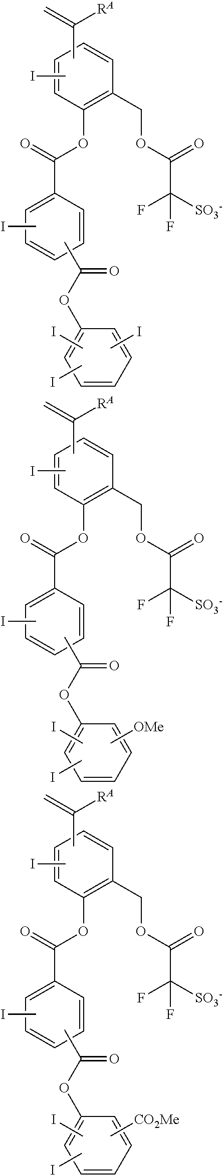

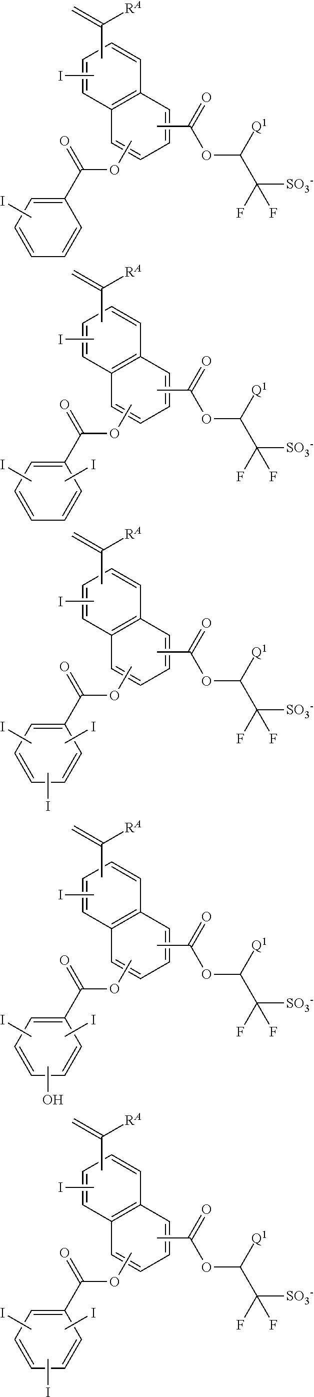

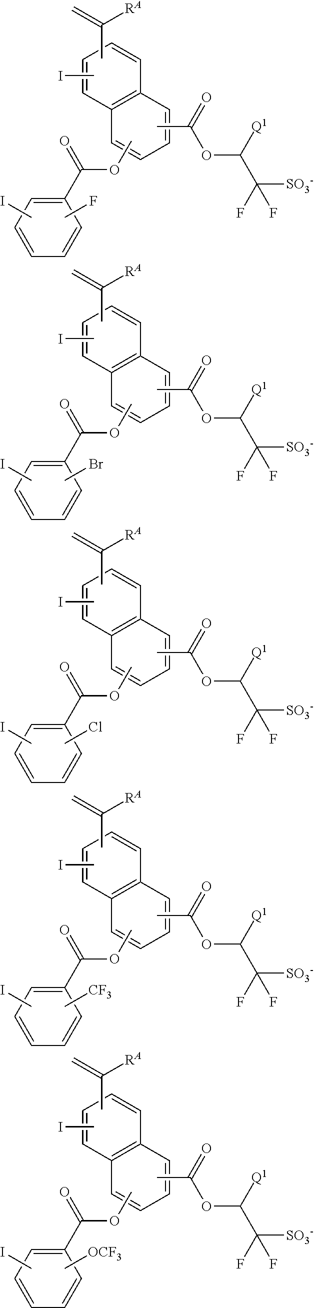

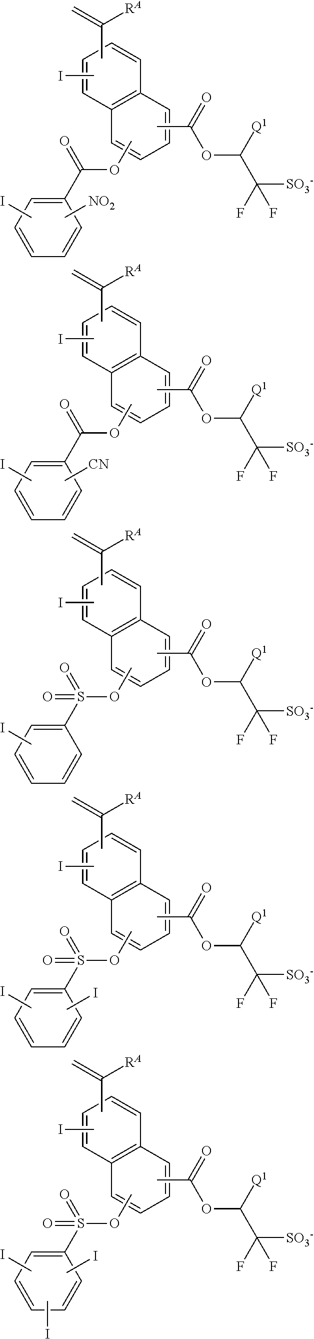

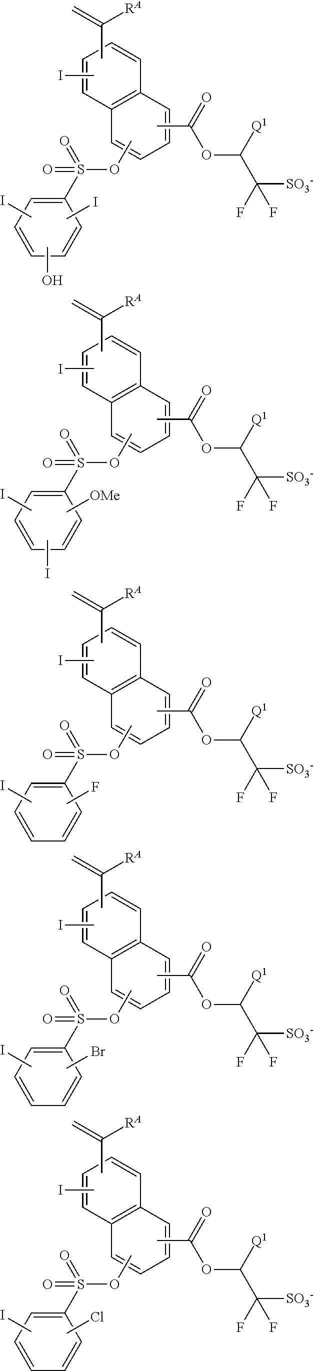

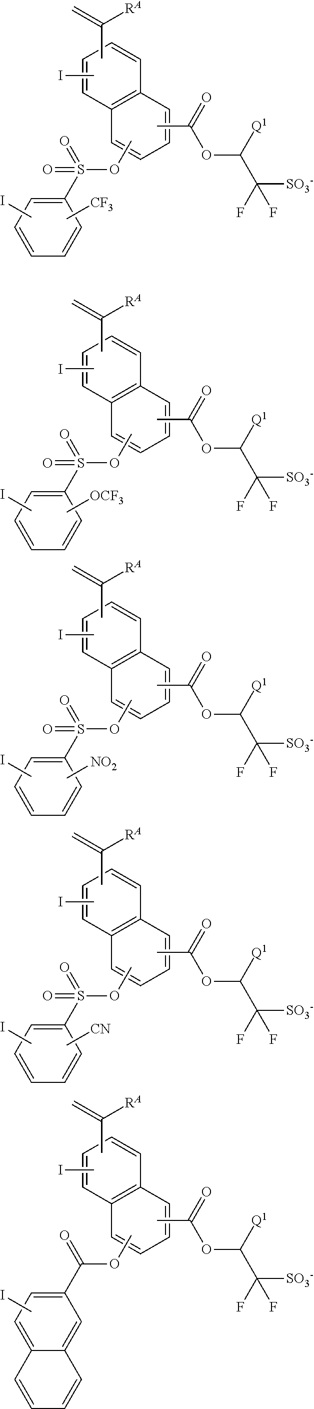

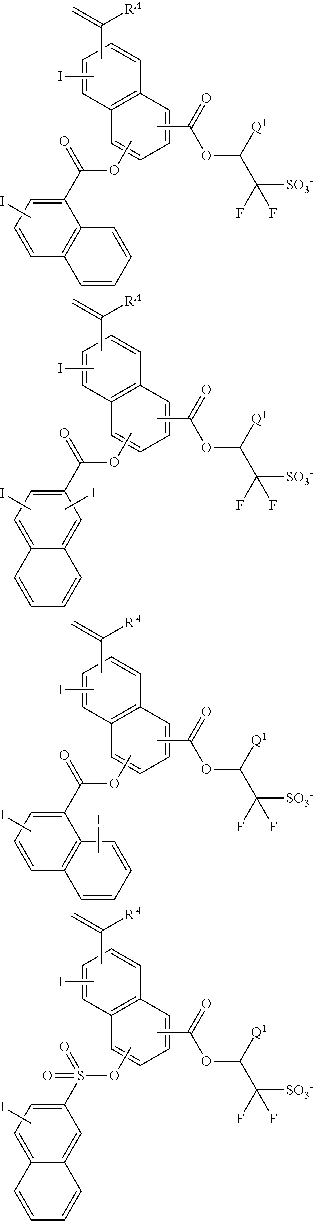

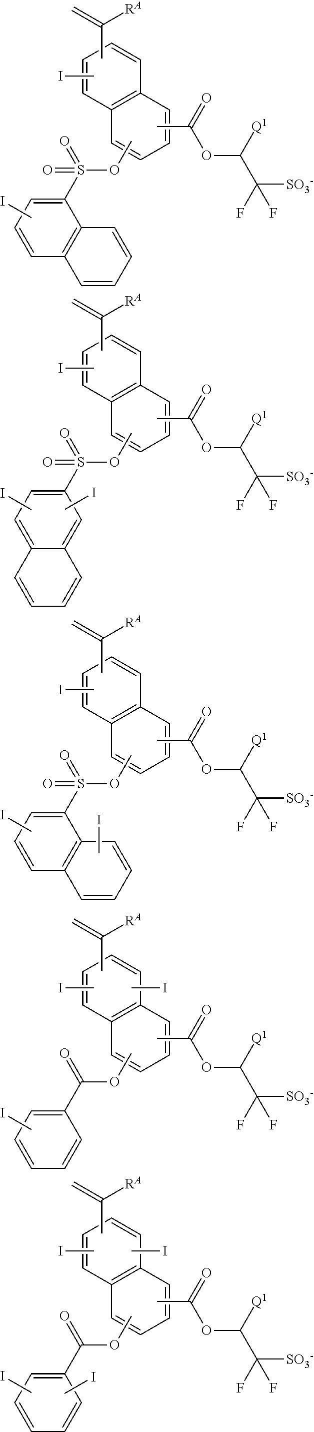

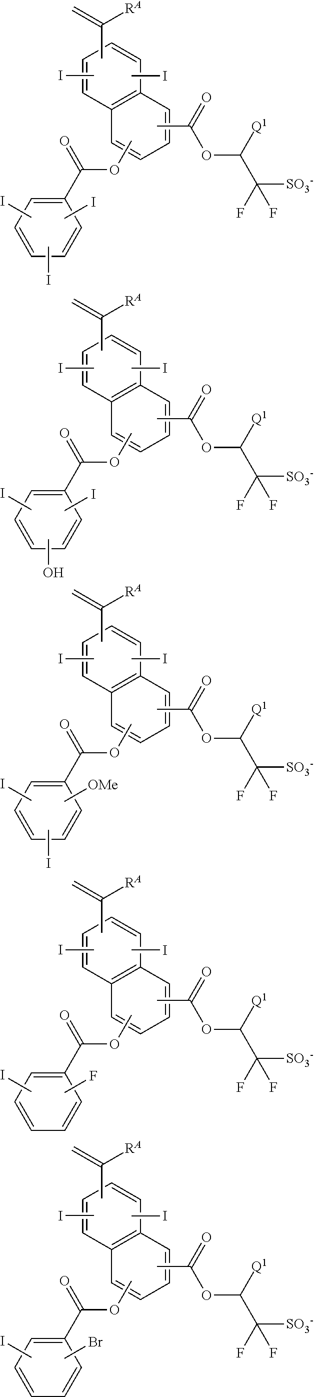

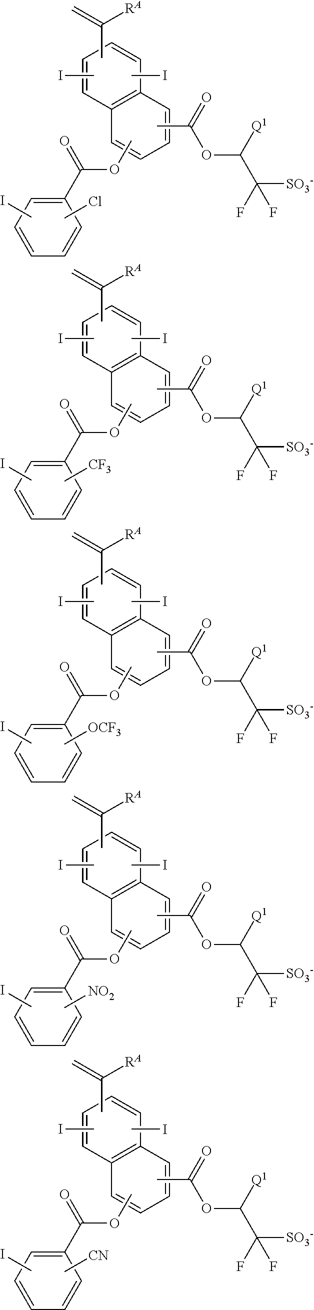

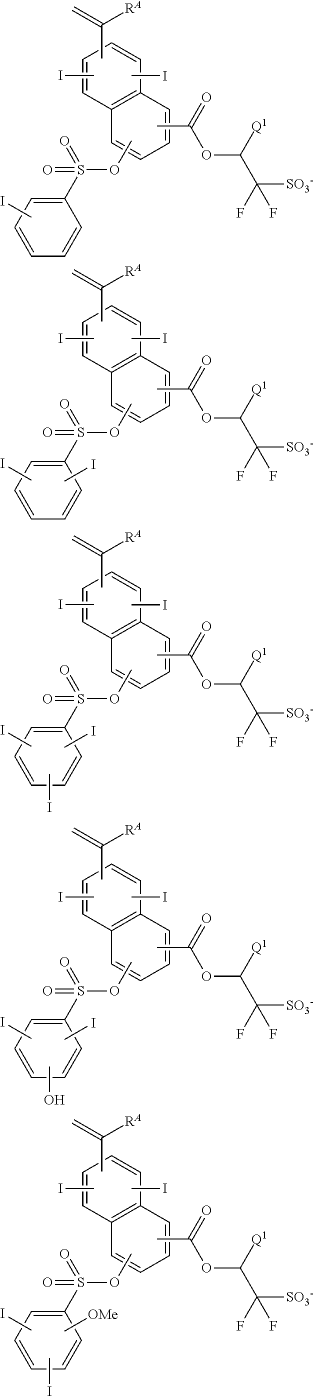

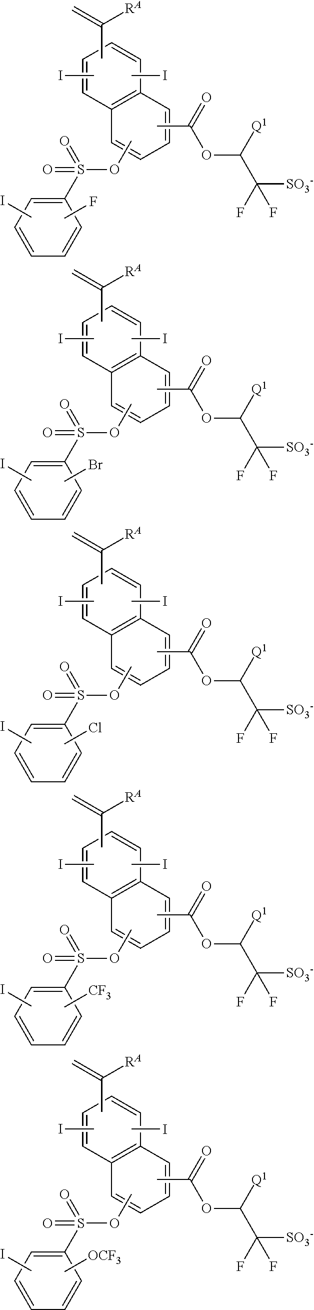

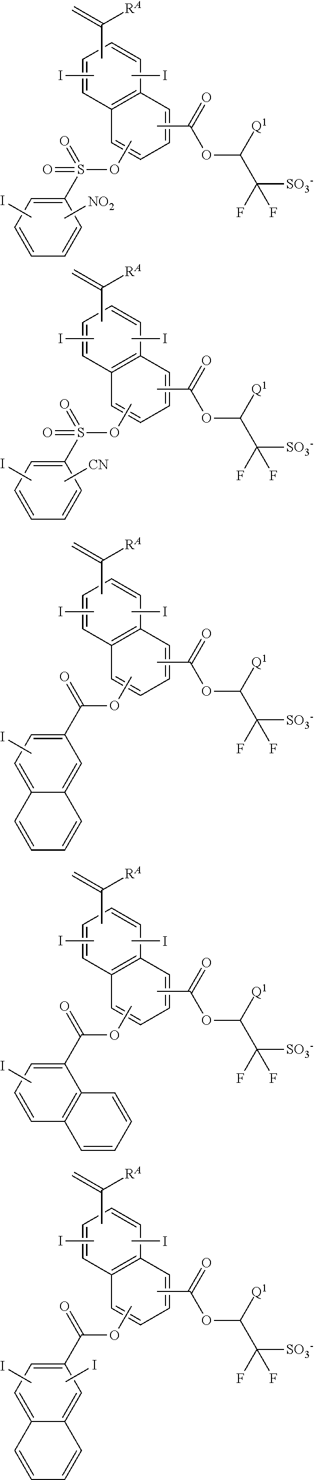

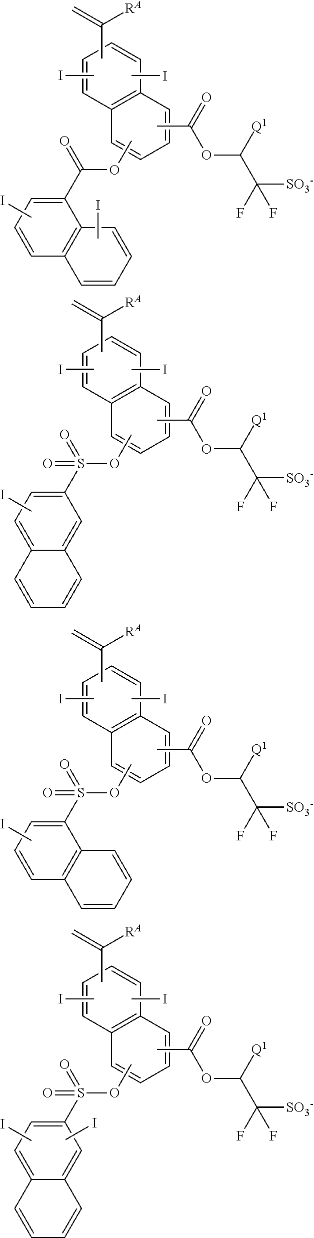

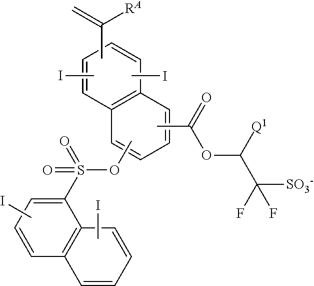

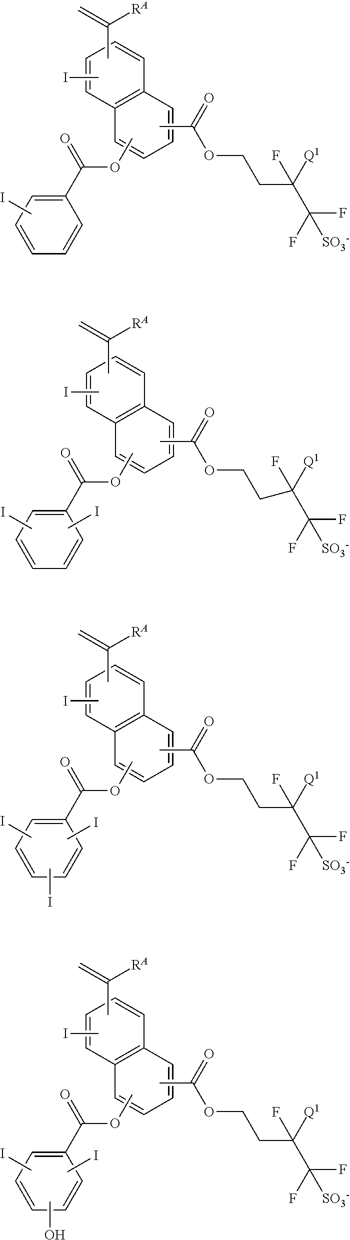

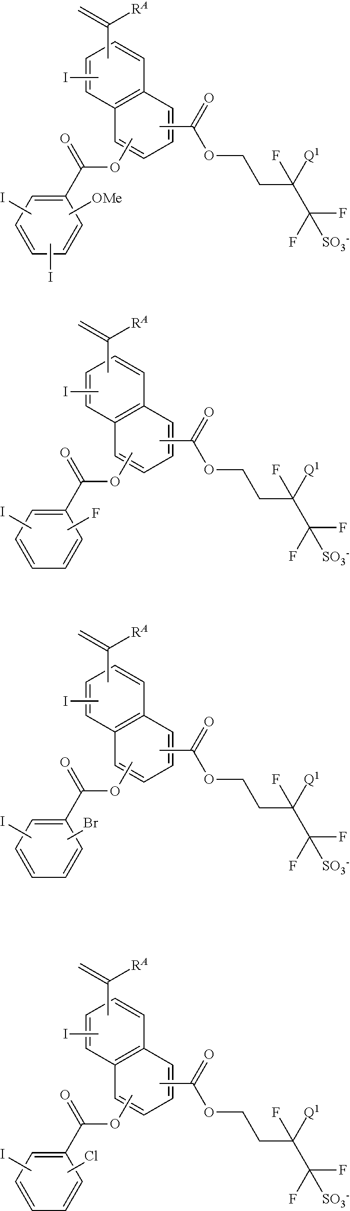

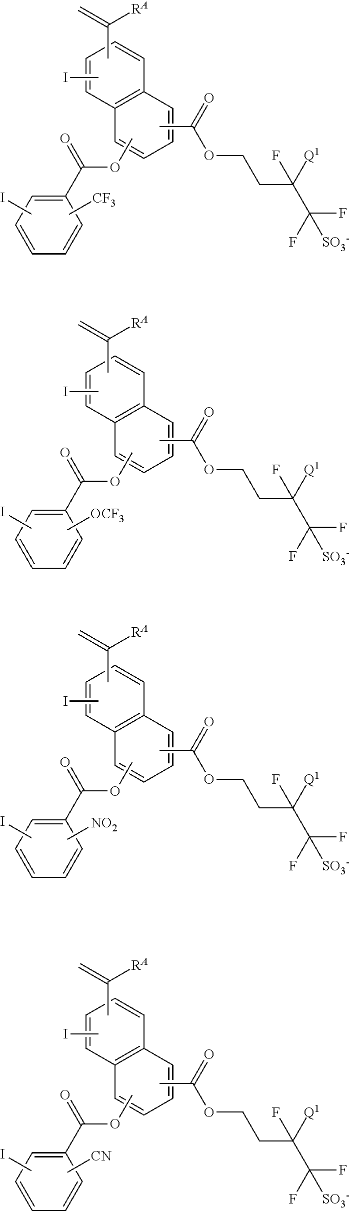

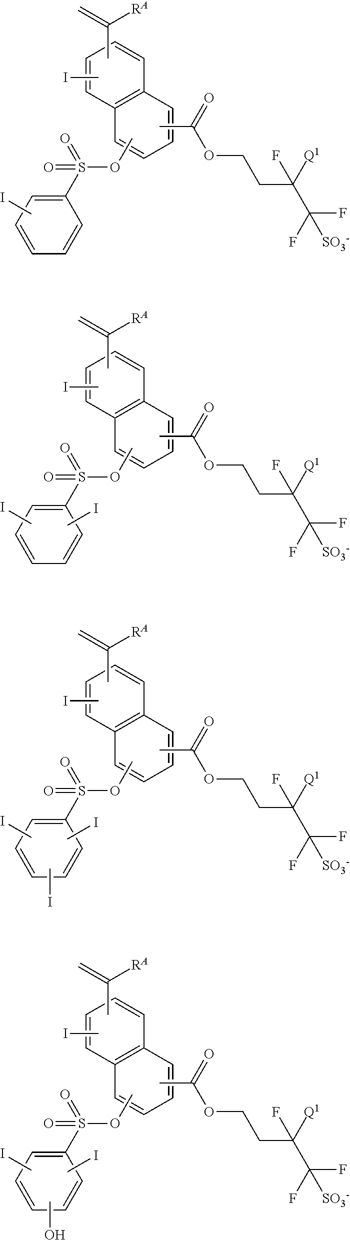

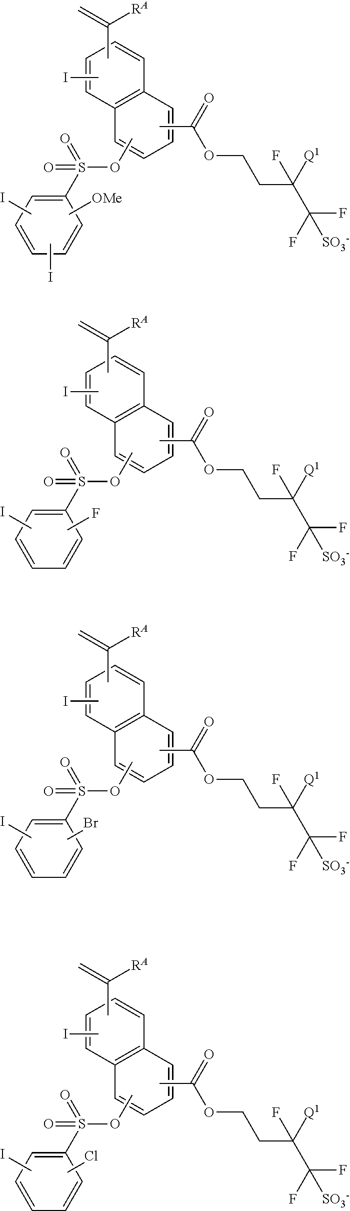

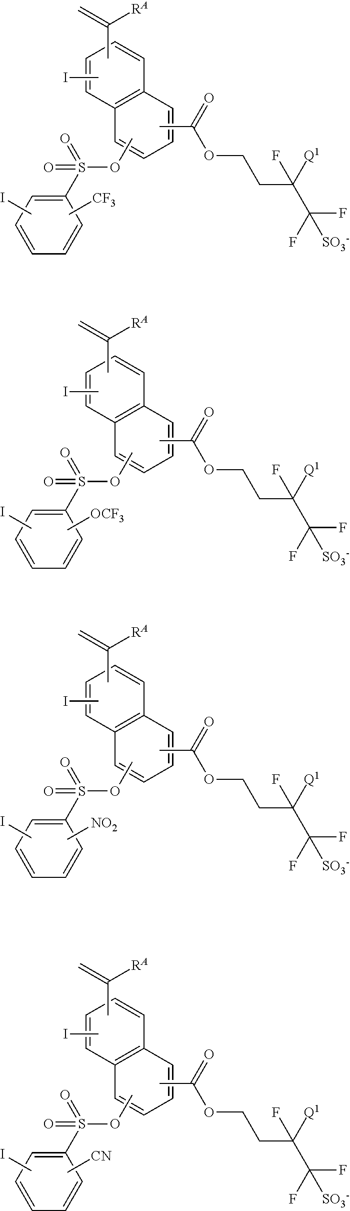

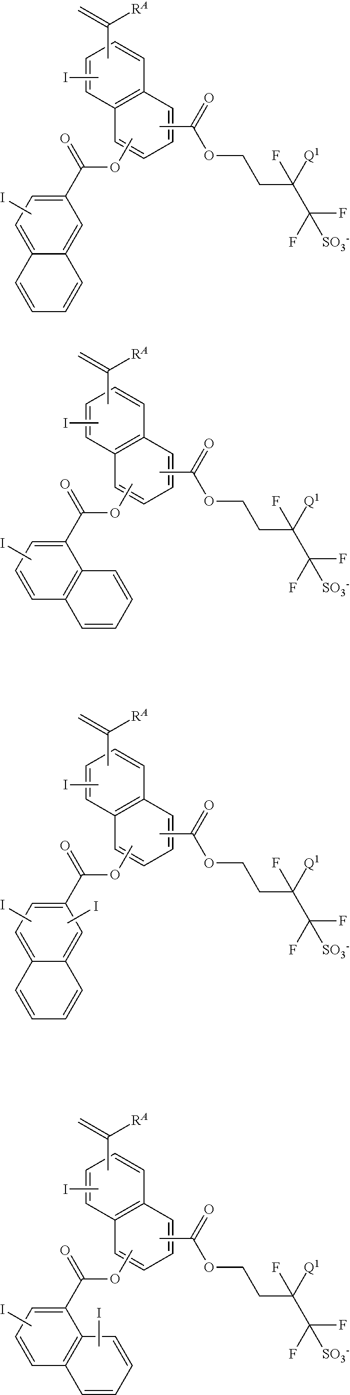

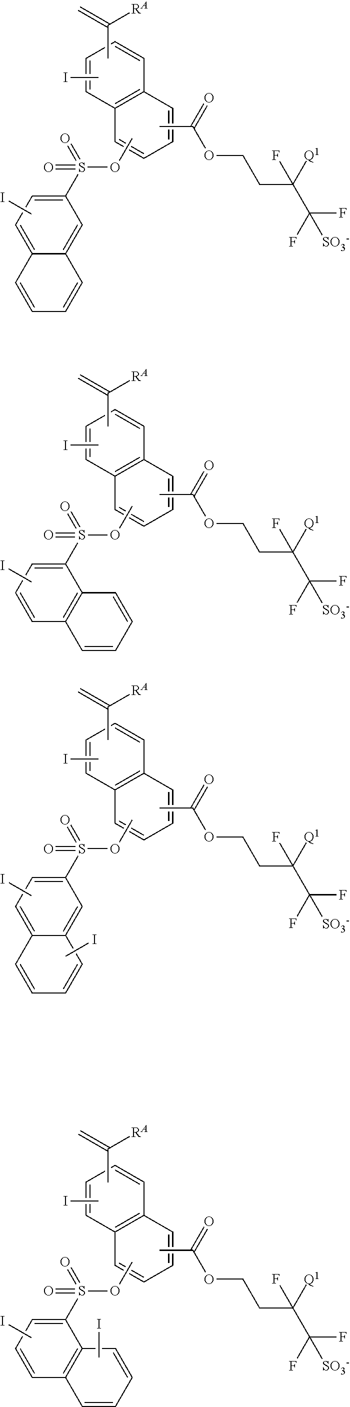

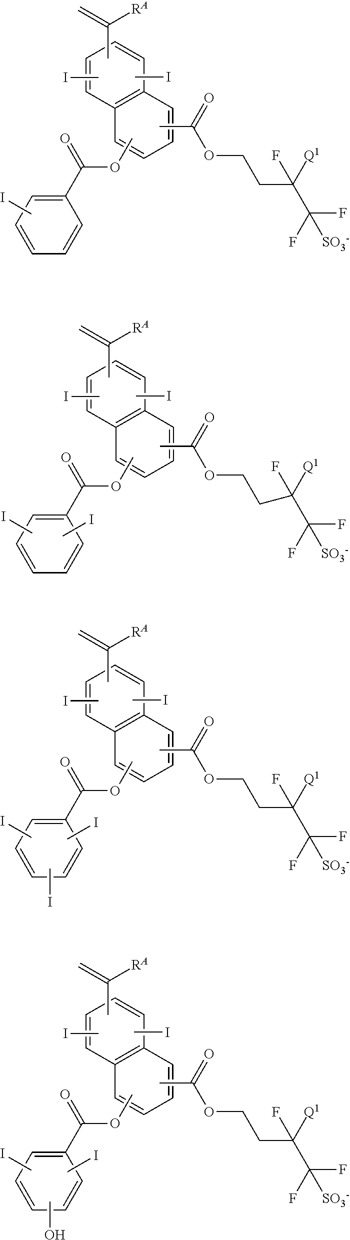

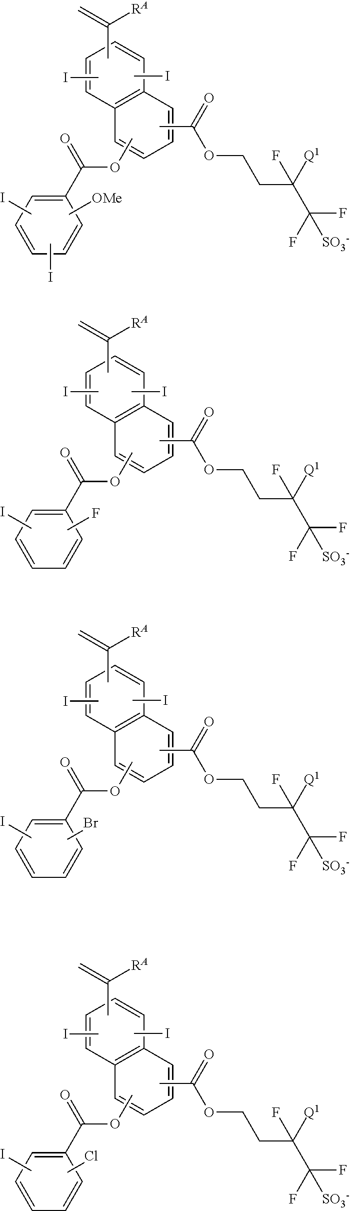

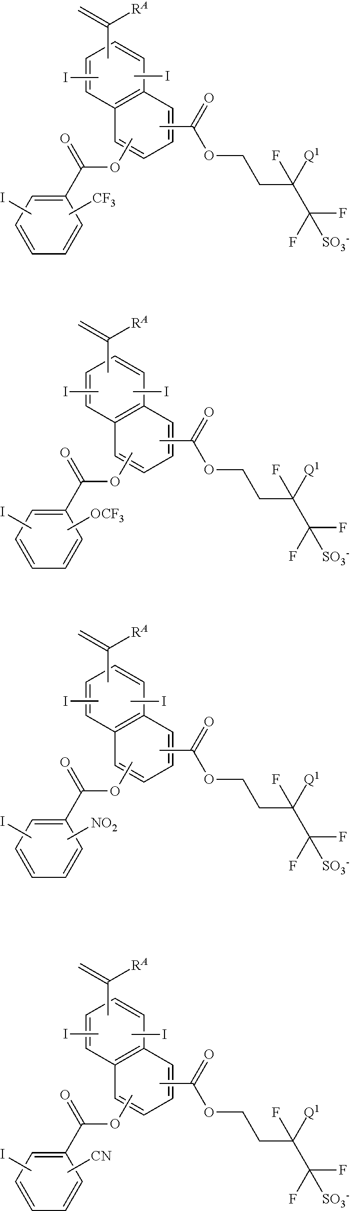

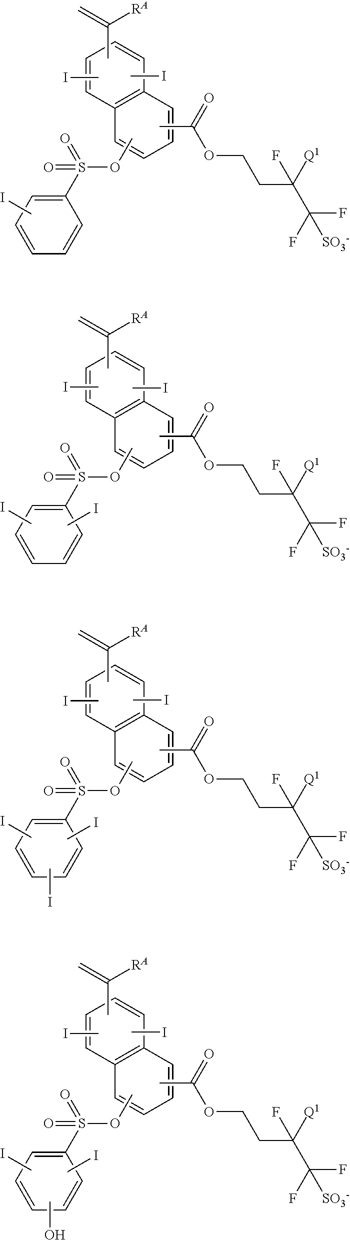

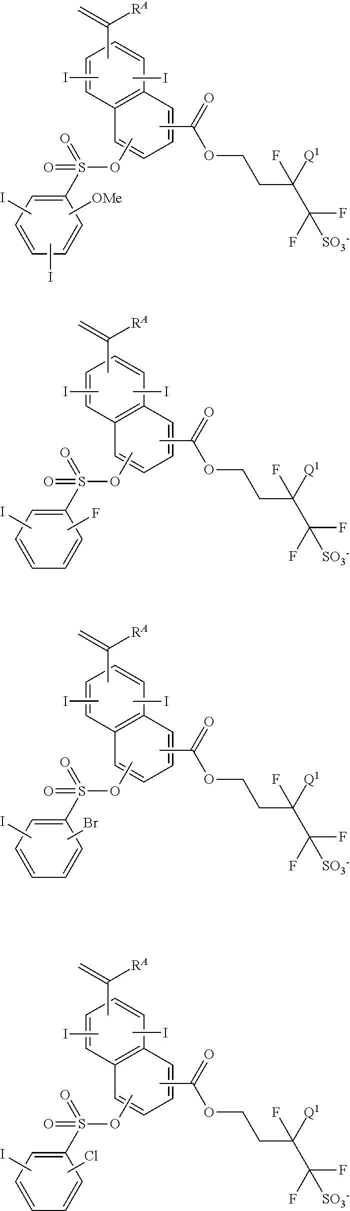

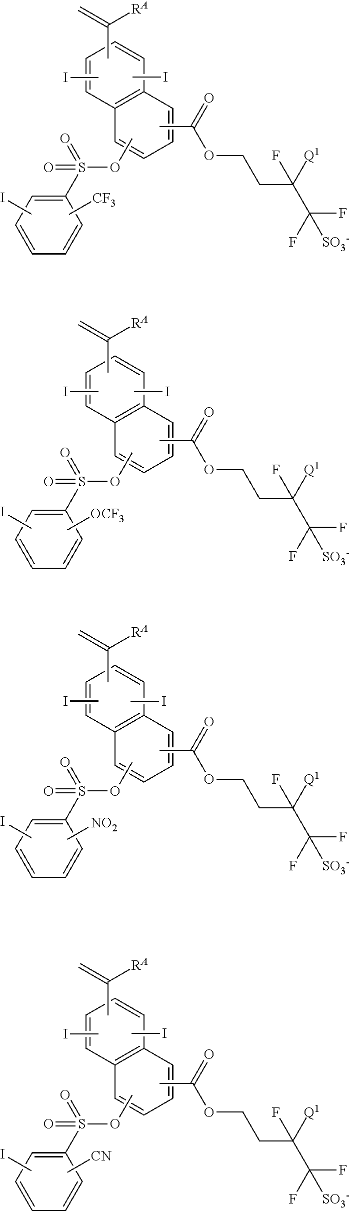

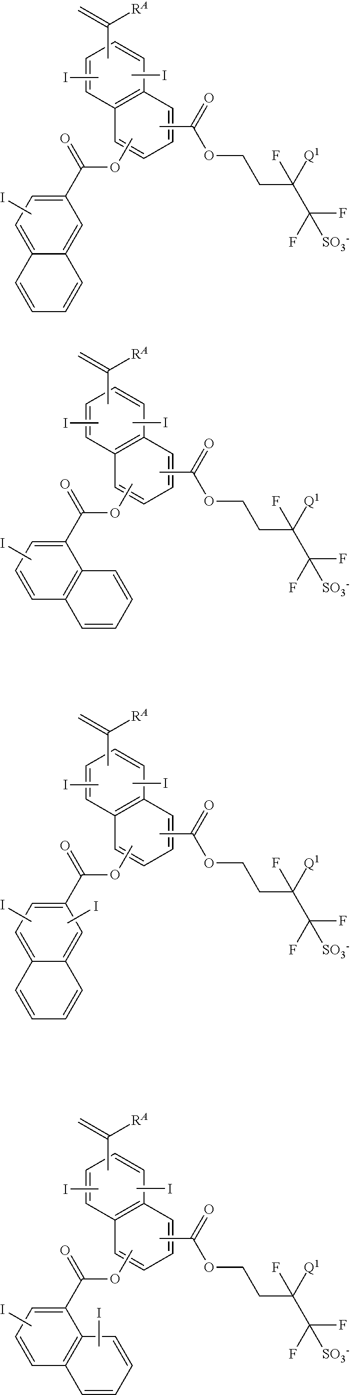

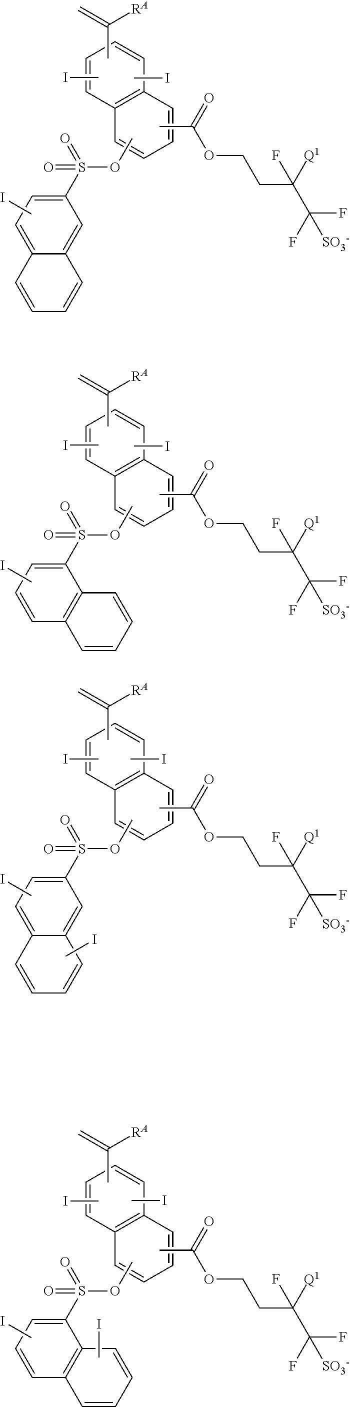

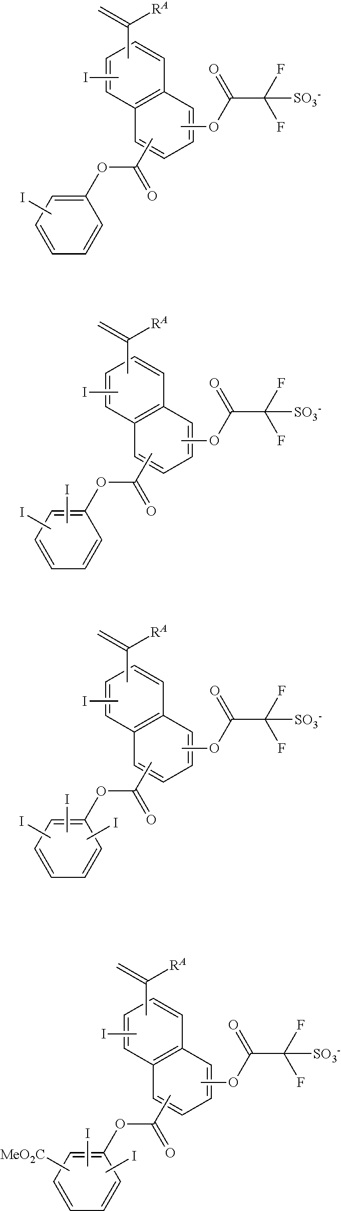

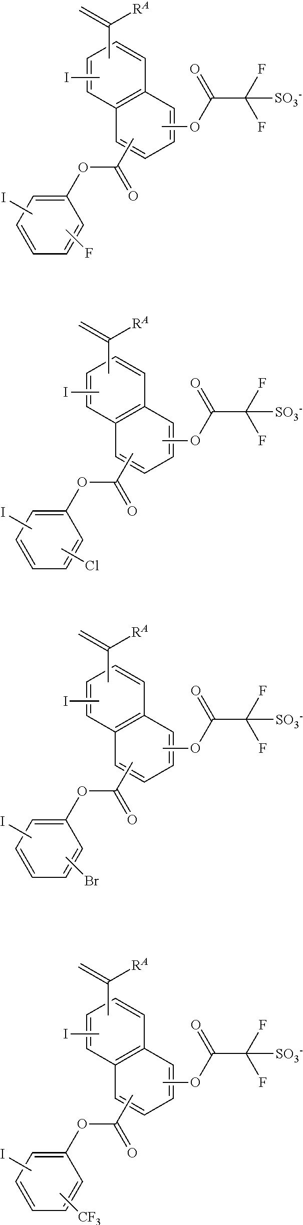

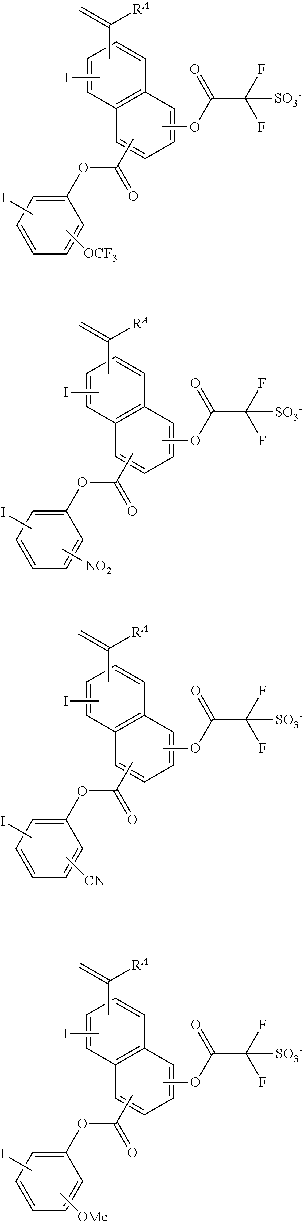

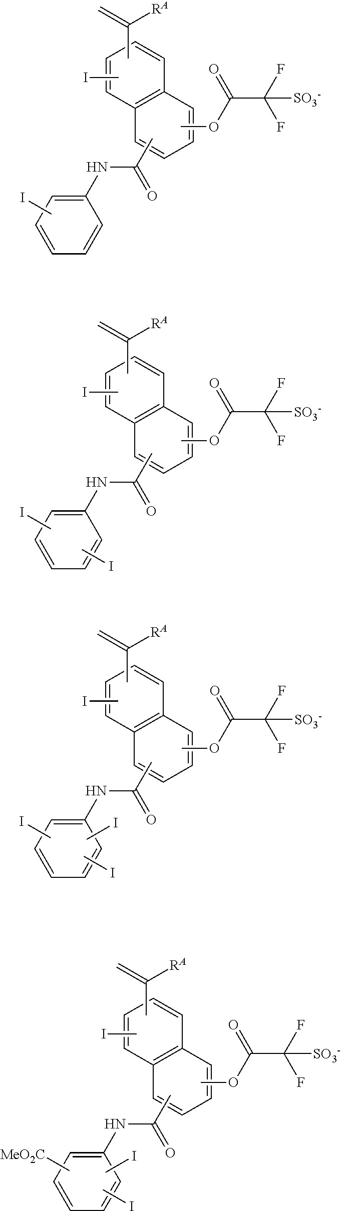

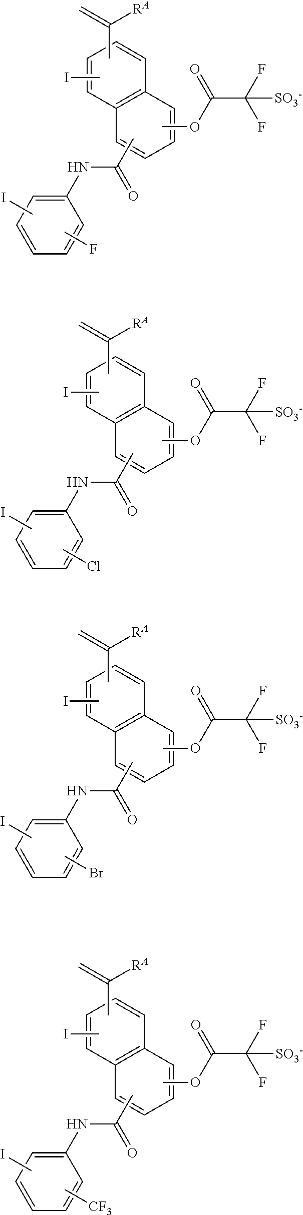

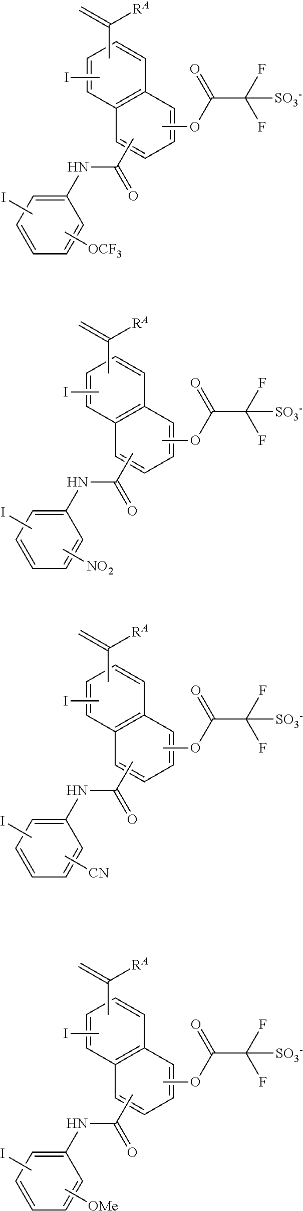

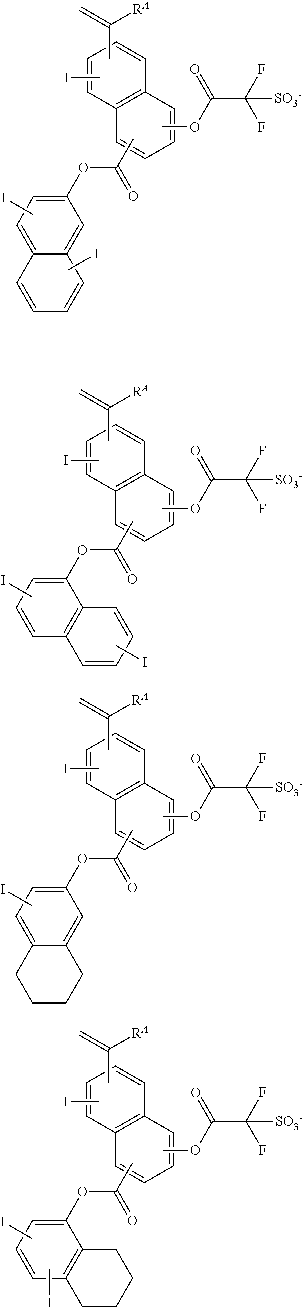

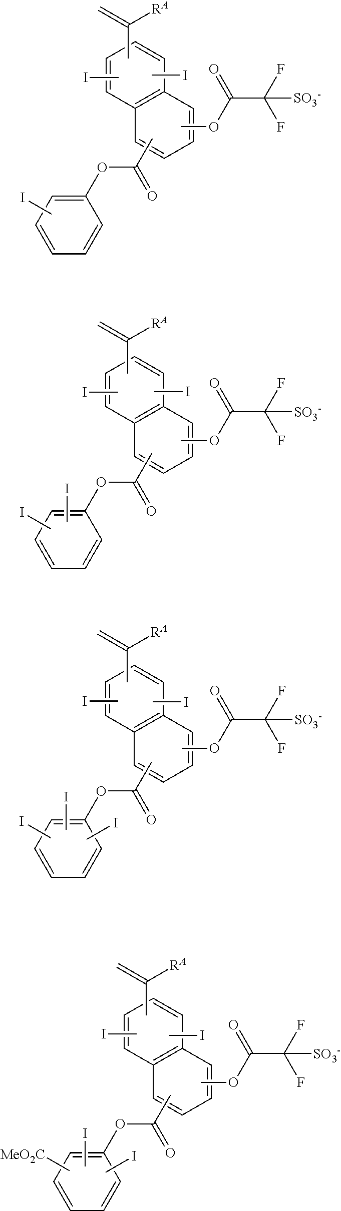

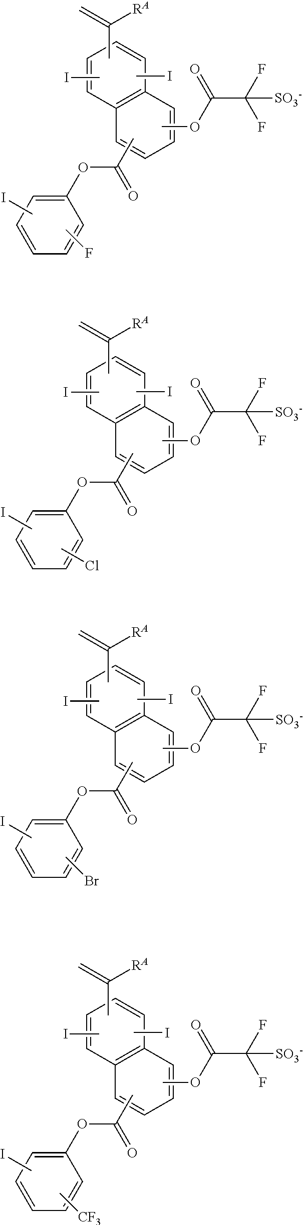

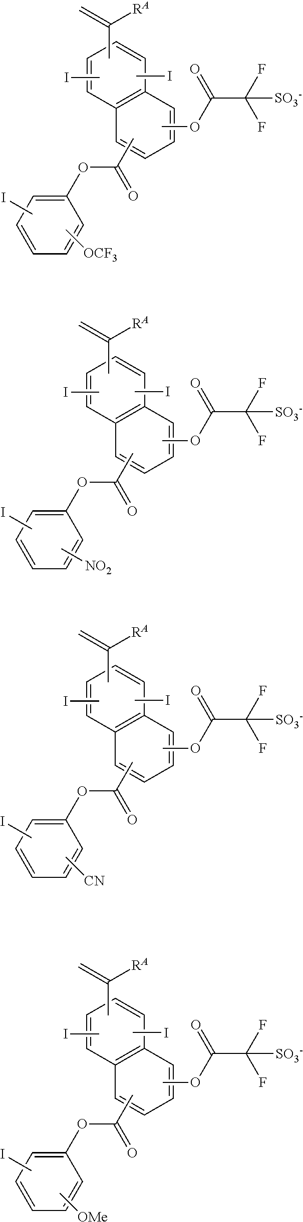

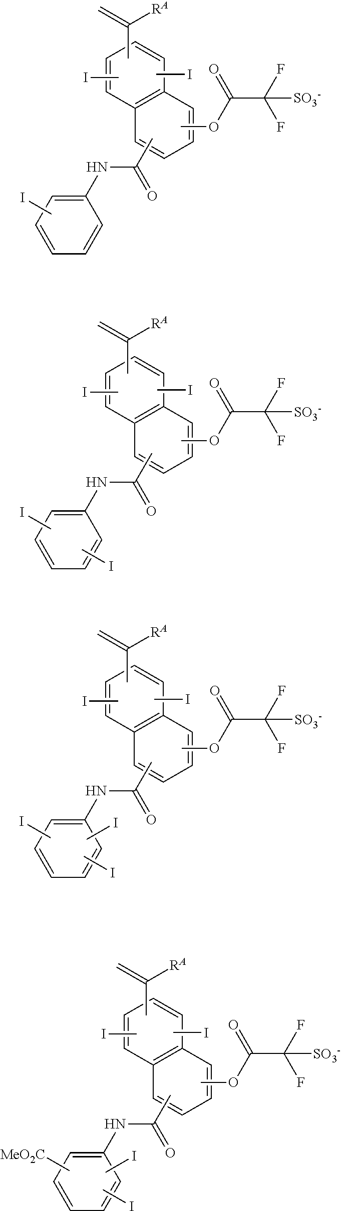

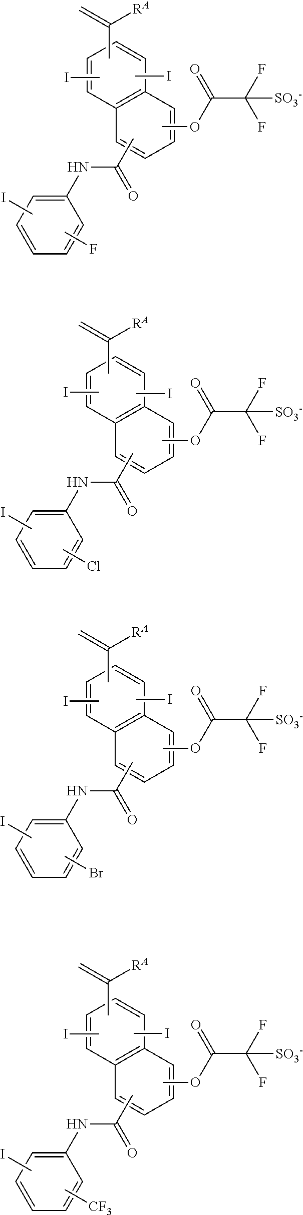

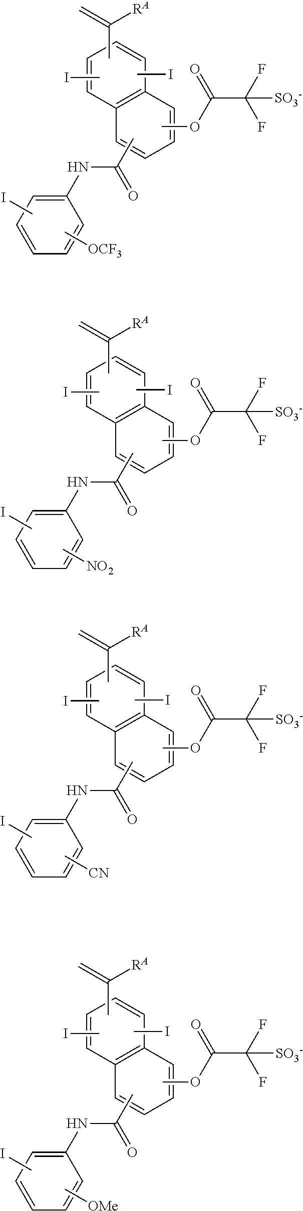

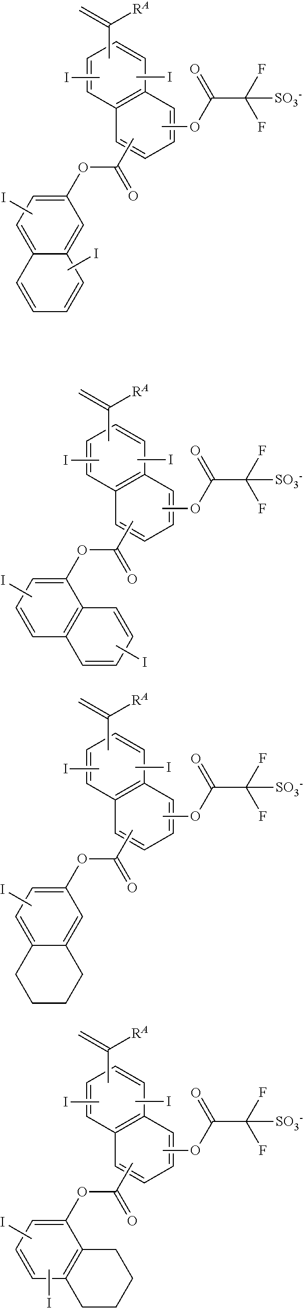

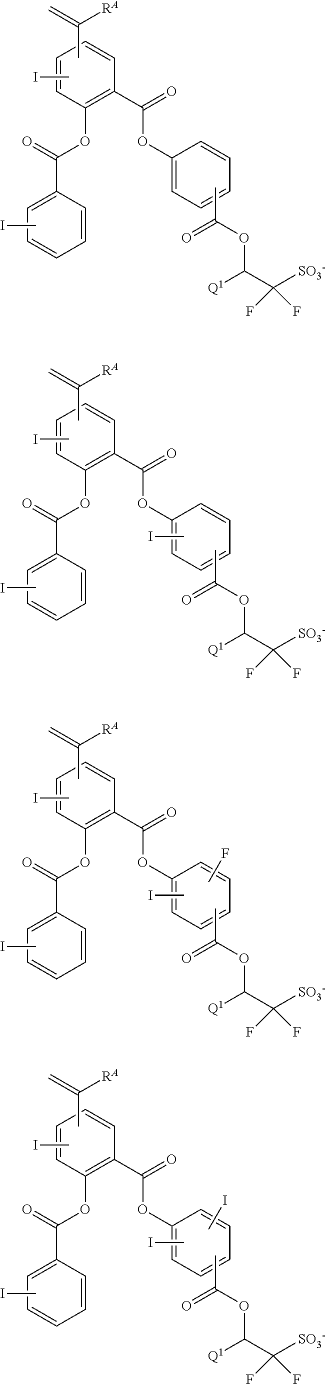

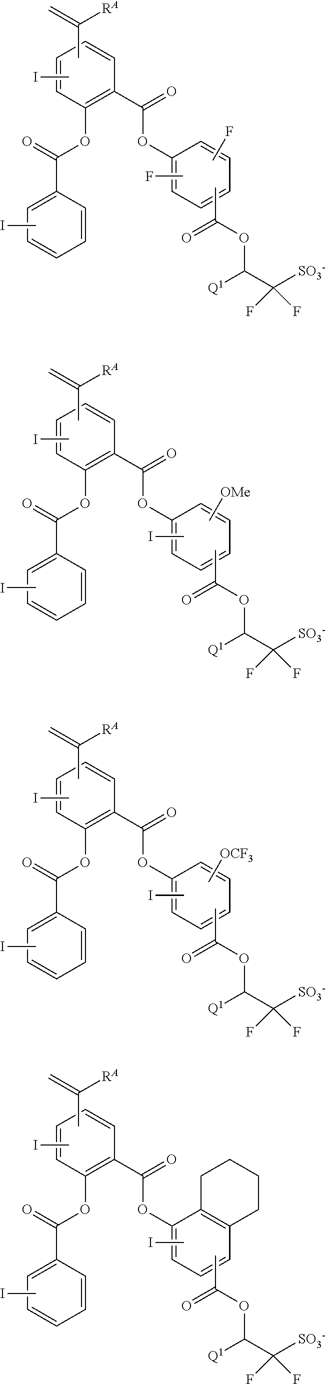

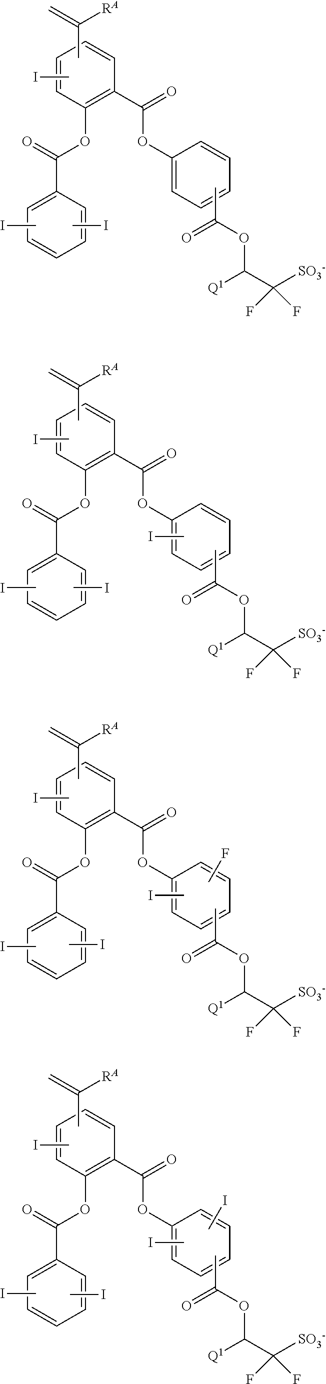

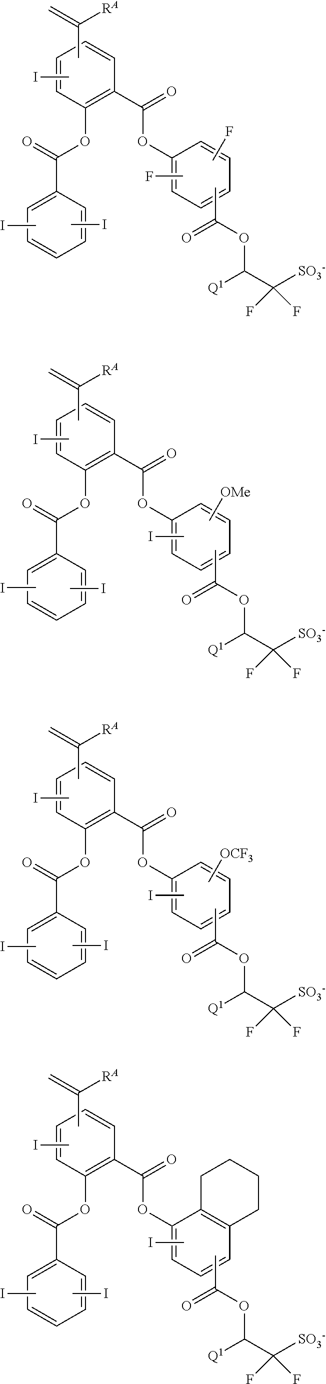

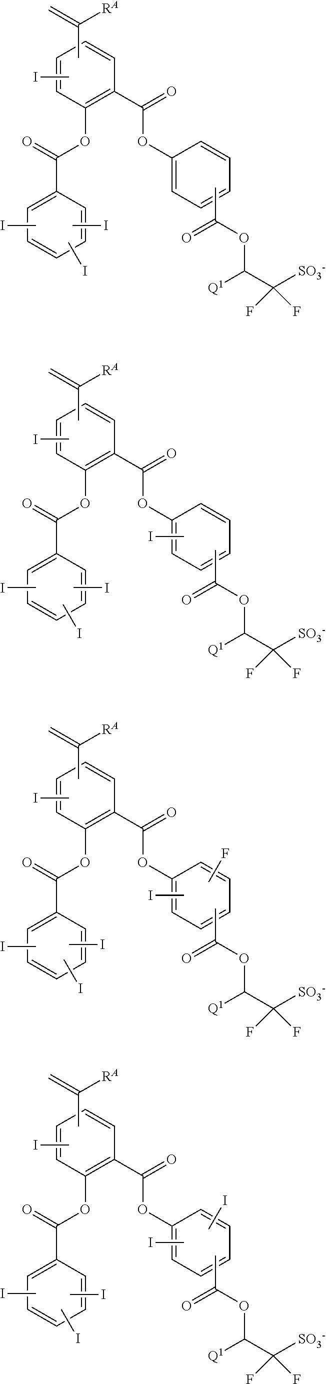

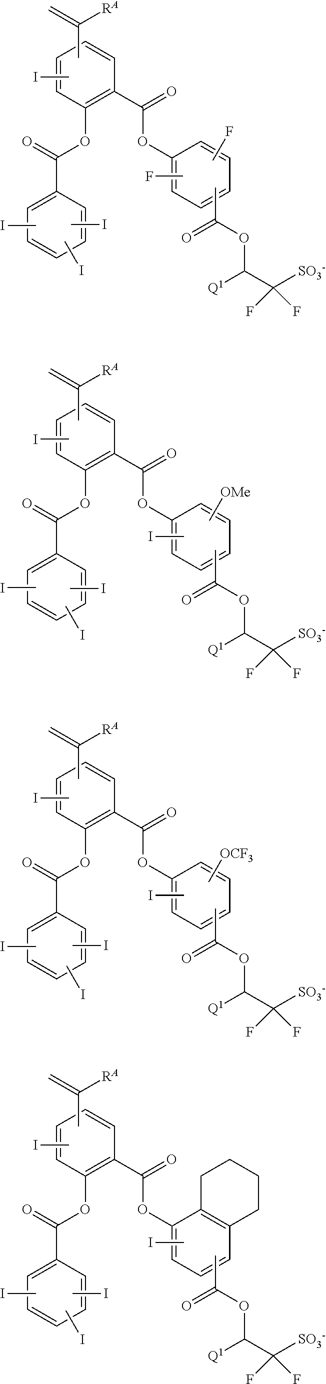

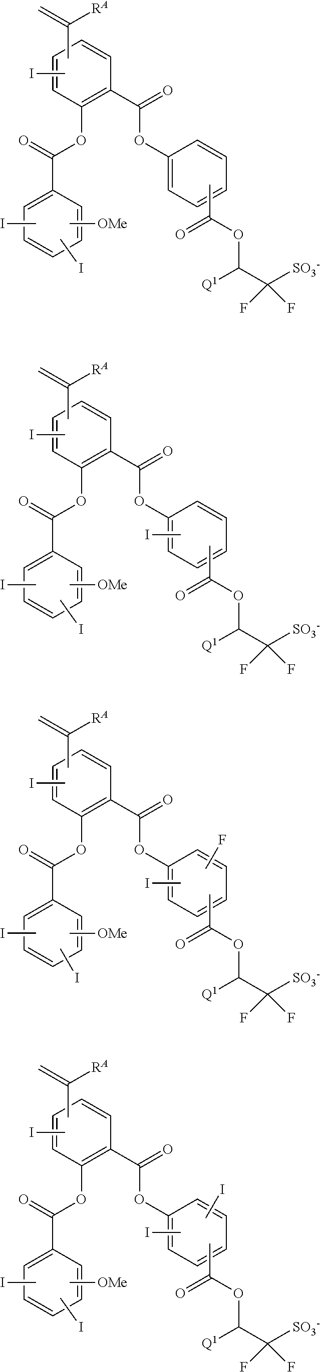

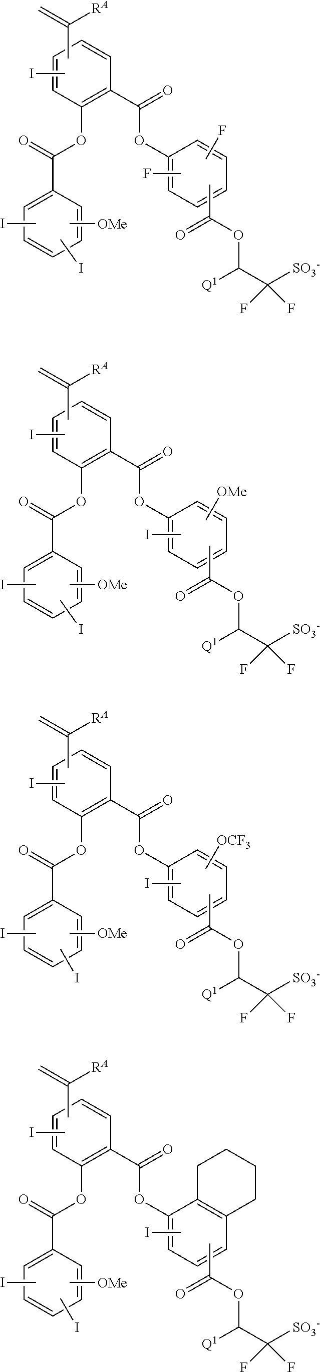

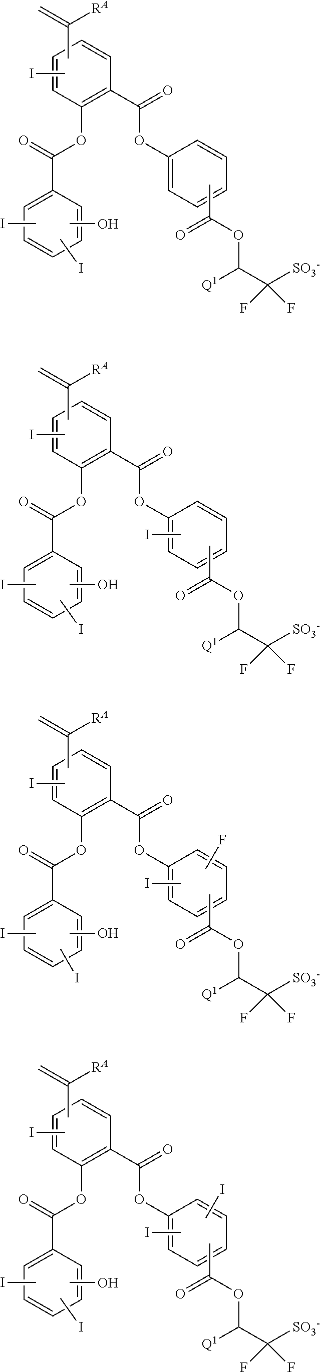

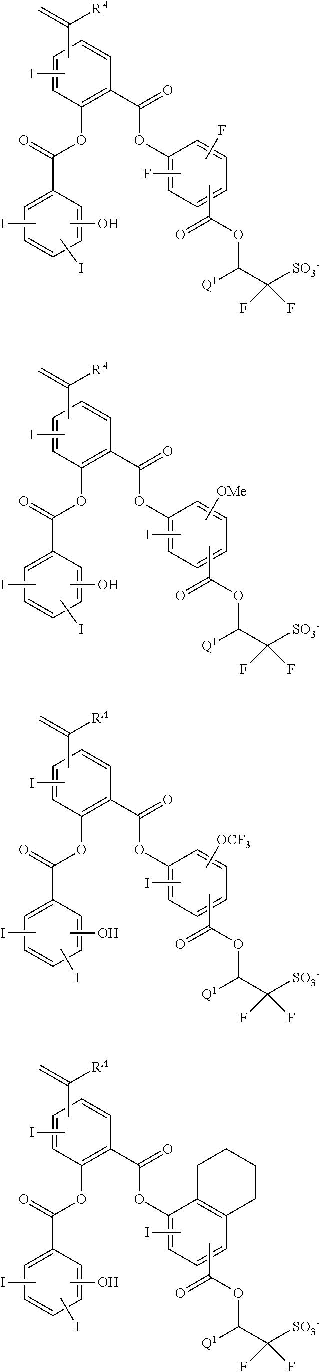

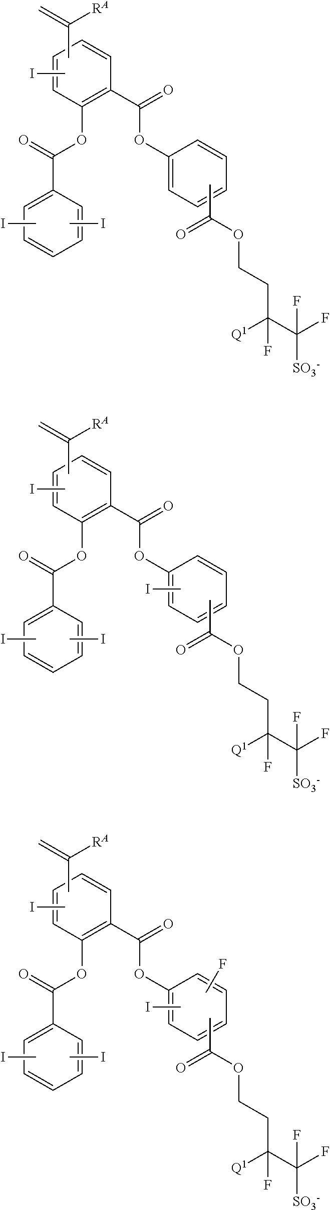

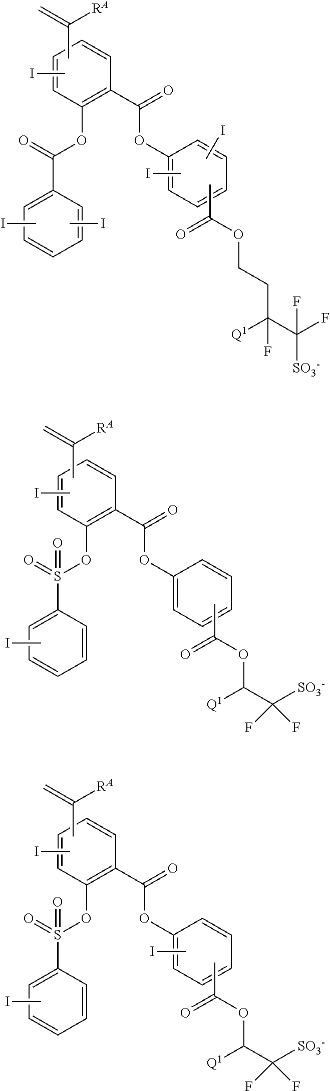

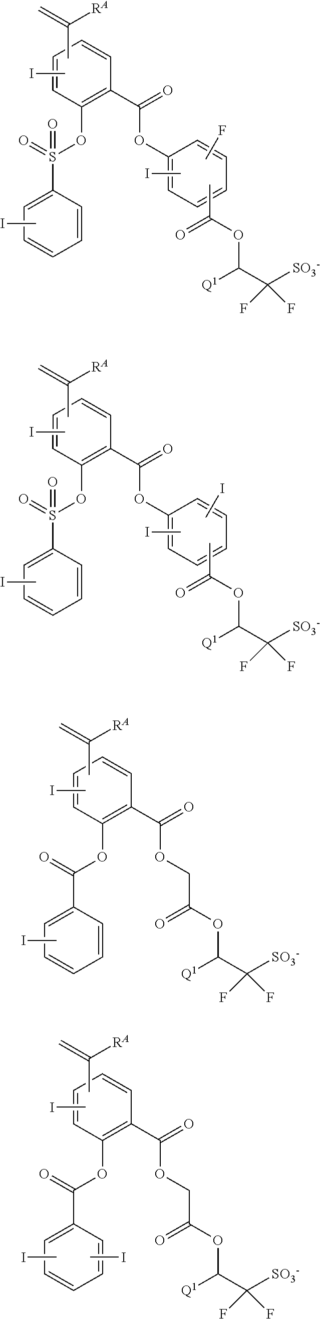

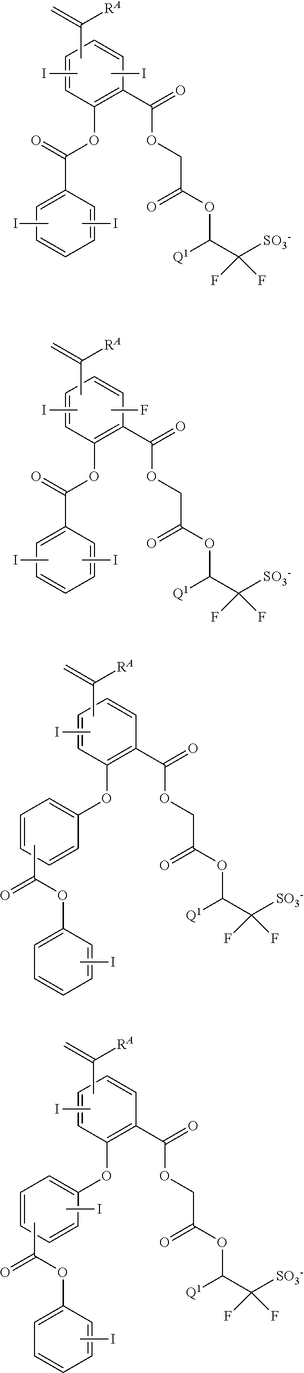

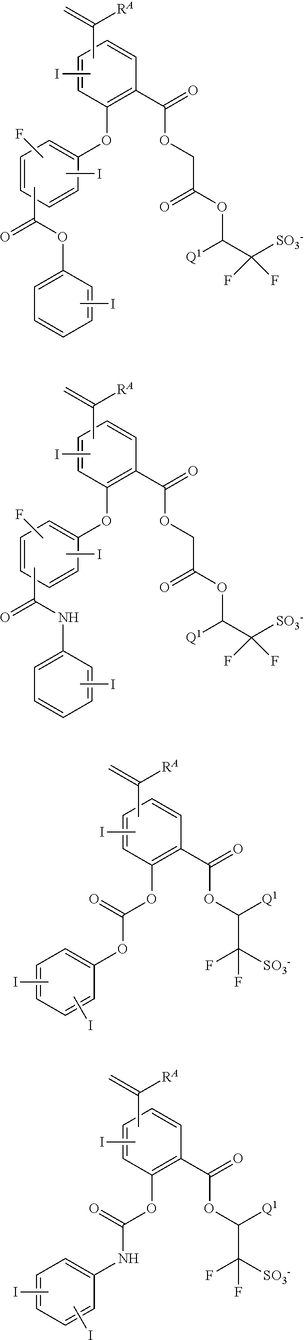

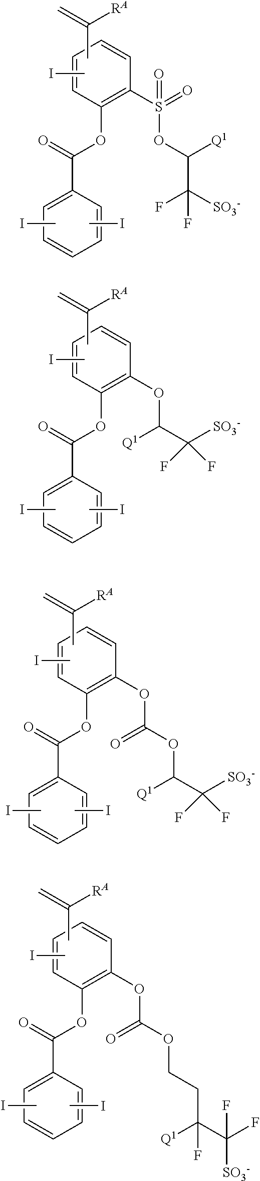

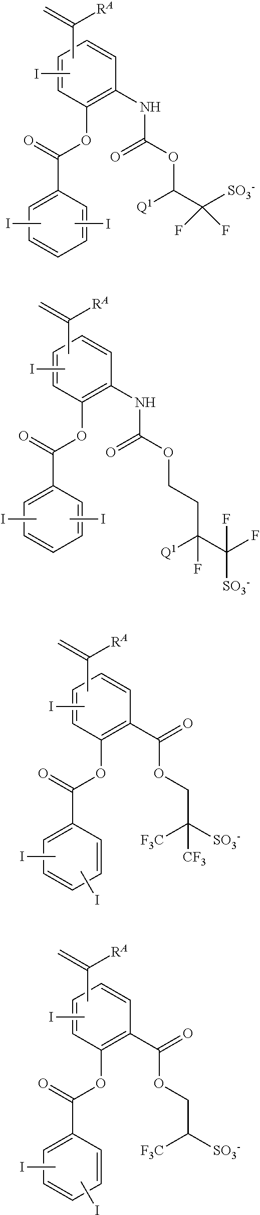

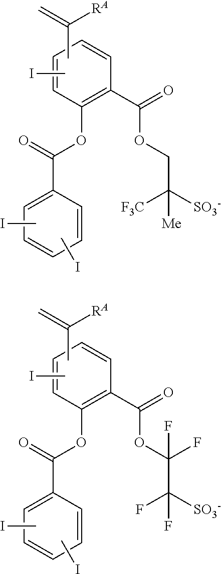

Exemplary structures of the sulfonium salt monomer of the invention include arbitrary combinations of anions with cations, both as exemplified above.

The sulfonium salt monomer of the invention can be synthesized by a known method. For example, first, a sulfonium salt containing the sulfonium cation is synthesized by the synthesis method described in ARKIVOC (Gainesville, FL, United States) (2022), (7), 7-18. Next, by subjecting the synthesized sulfonium salt and the corresponding anion to a salt exchange reaction, the sulfonium salt can be converted into an intended sulfonium salt. The salt exchange with the corresponding anion can be easily performed by a known method, and reference may be made to JP-A 2007-145797, for example.

The above production method is merely an example, and the method for producing the sulfonium salt of the invention is not limited thereto.

Examples of the structural characteristics of the sulfonium salt monomer of the invention include a nitro group bonded to an aromatic ring of a sulfonium cation together with an aromatic vinyl structure as a polymerizable group and a fluorosulfonate anion structure having an iodine atom. Iodine which is outstandingly absorptive to EUV of wavelength 13.5 nm generates secondary electrons during EUV exposure. Since the sulfonium salt monomer of the invention has a polymerizable group in an anionic part, the polymer of the invention obtained using the sulfonium salt monomer becomes an anion-bound acid generator bonded to the polymer backbone in the anionic side. That is, since an acid bonded to the backbone of the polymer is generated, diffusion of the generated acid can be suppressed. In particular, a polymerizable group having a styrene or vinylnaphthalene structure is more rigid than a polymerizable group such as methacrylate and improves the glass transition temperature (Tg) of the polymer. It is considered that the aromatic rings in the polymer or between the polymers interact with each other (x-T stacking effect), so that the polymers are regularly arranged, and resistance to pattern collapse is developed against a developer even upon formation of small-size patterns. In an etching step after small-size pattern formation, excellent etching resistance is developed by having an aromatic ring directly connected to the backbone. On the other hand, a nitro group substituted on the aromatic ring of a sulfonium cation is known as a strong electron-withdrawing group. Thus, it is considered that the energy level of the LUMO in frontier molecular orbital theory is lowered. Therefore, the secondary electrons generated from the iodine atom in the anion are easily received, the decomposition of the cation is promoted, and an acid is efficiently generated. In addition, since the nitro group has a resonance structure in which the nitrogen atom is positively charged and one oxygen atom is negatively charged, the nitro group interacts with a proton of the generated acid at a negative charge on the oxygen atom, and a function as an acid diffusion inhibiting group can also be expected. By being charged as described above, an affinity with an alkaline developer is also high, and the residue after cationic decomposition is efficiently removed with respect to the alkaline developer, so that the risk of development defects in the exposed region is also reduced. The sensitivity is increased by these synergistic effects, and it is possible to prevent a reduction of resolution due to blur by acid diffusion and to improve the LWR and CDU. Thus, the polymers of the invention are suitable in particular as materials for chemically amplified positive resist compositions.

### Polymer

The polymer of the invention comprise repeat units derived from a sulfonium salt monomer having the formula (A), which are also referred to as repeat units (A).

The polymer may comprise repeat units having the formula (a1), which are also referred to as repeat units (a1) or repeat units having the formula (a2), which are also referred to as repeat units (a2).

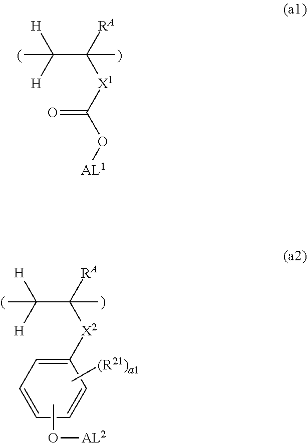

In the formulae (a1) and (a2), R^{A} is each independently hydrogen, fluorine, methyl or trifluoromethyl.

In the formula (a1), X¹ is a single bond, phenylene group, naphthylene group, or *—C(═O)—O—X¹¹—. The phenylene or naphthylene group may be substituted by a hydroxy moiety, nitro moiety, cyano moiety, optionally fluorinated C₁-C₁₀ saturated hydrocarbyl moiety, optionally fluorinated C₁-C₁₀ saturated hydrocarbyloxy moiety, or halogen. X¹¹ is a C₁-C₁₀ saturated hydrocarbylene group which may contain a hydroxy moiety, ether bond, ester bond or lactone ring, or phenylene or naphthylene group. The asterisk (*) designates a point of attachment to the carbon atom in the backbone.

In the formula (a2), X² is a single bond or *—C(—O)—O—. The asterisk (*) designates a point of attachment to the carbon atom in the backbone. R²¹ is halogen, cyano, hydroxy, nitro, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. a1 is 0, 1, 2, 3, or 4, preferably 0 or 1. A plurality of R²¹ may be identical or different when a1 is 2, 3, or 4.

In the formulae (a1) and (a2), AL¹ and AL² are each independently an acid labile group. Examples of the acid labile group include those described in JP-A 2013-080033 and JP-A 2013-083821.

Typical examples of the acid labile groups include groups having the formulae (AL-1) to (AL-3):

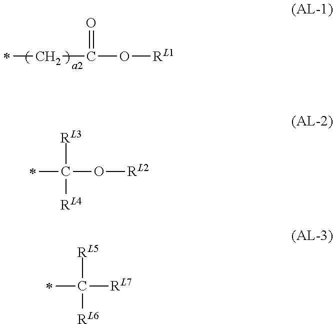

wherein the asterisk (*) is a valence bond.

In the formulae (AL-1) and (AL-2), R^{L1} and R¹² are each independently a C₁-C₄₀ hydrocarbyl group which may contain a heteroatom such as oxygen, sulfur, nitrogen or fluorine. The hydrocarbyl group may be saturated or unsaturated and straight, branched or cyclic. The hydrocarbyl group is preferably a C₁-C₂₀ hydrocarbyl group.

In the formula (AL-1), a2 is an integer of 0 to 10, preferably 1, 2, 3, 4, or 5.

In the formula (AL-2), R^{L3} and R^{L4} are each independently hydrogen or a C₁-C₂₀ hydrocarbyl group which may contain a heteroatom such as oxygen, sulfur, nitrogen or fluorine. The hydrocarbyl group may be saturated or unsaturated and straight, branched or cyclic. The hydrocarbyl group is preferably a C₁-C₂₀ hydrocarbyl group. Any two of R^{L2}, R^{L3}, and R^{L4} may bond together to form a C₃-C₂₀ ring with the carbon atom or carbon and oxygen atoms to which they are attached. The ring is preferably a C₄-C₁₆ ring, particularly preferably an alicyclic ring.

In the formula (AL-3), R¹⁵, R¹⁶, and R¹⁷ are each independently a C₁-C₂₀ hydrocarbyl group which may contain a heteroatom such as oxygen, sulfur, nitrogen or fluorine. The hydrocarbyl group may be saturated or unsaturated and straight, branched or cyclic. The hydrocarbyl group is preferably a C₁-C₂₀ hydrocarbyl group. Any two of R^{L5}, R^{L6}, and R^{L7} may bond together to form a C₃-C₂₀ ring with the carbon atom to which they are attached. The ring is preferably a C₄-C₁₆ ring, particularly preferably an alicyclic ring.

Examples of the repeat units (a1) include those shown below, but not limited thereto. In the formulae, R^{A} and AL¹ are as defined above.

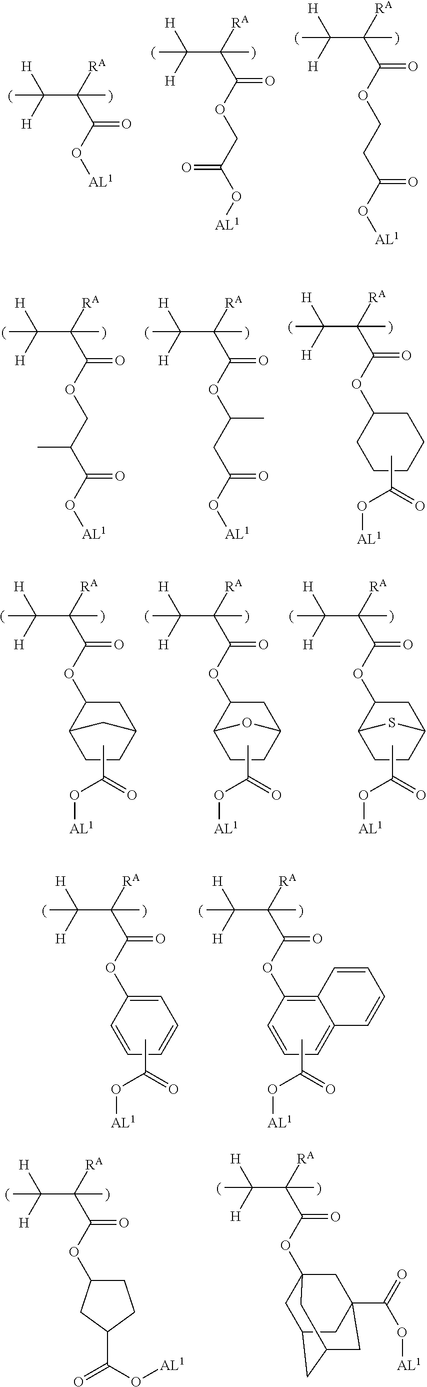

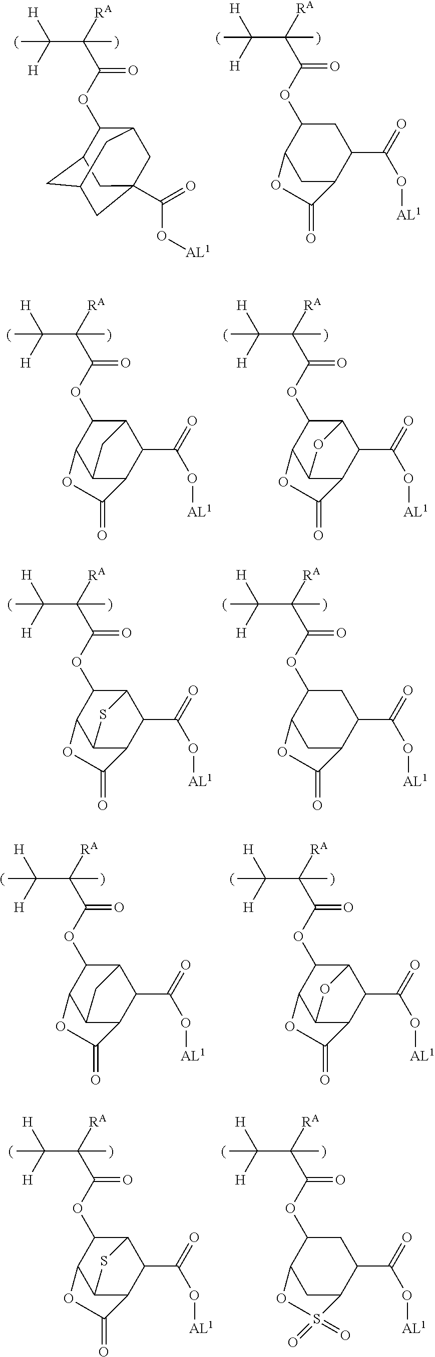

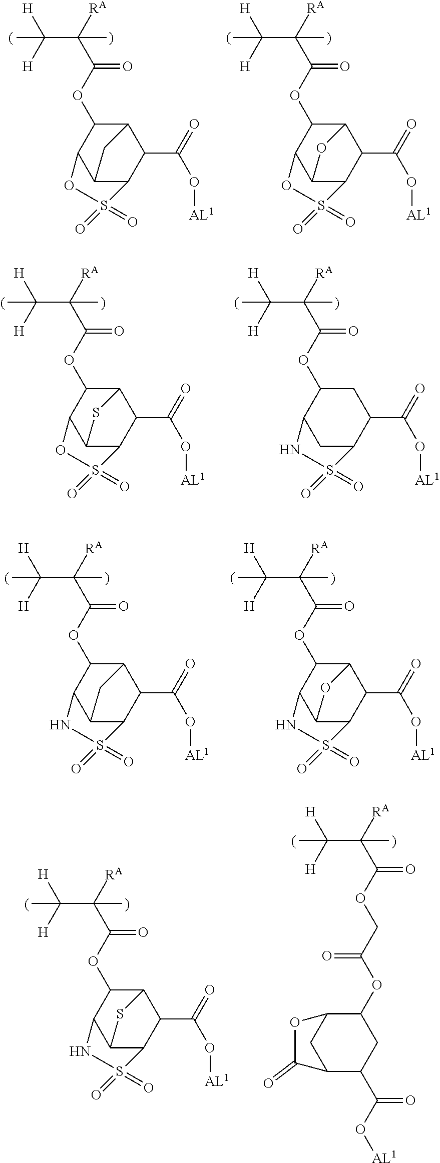

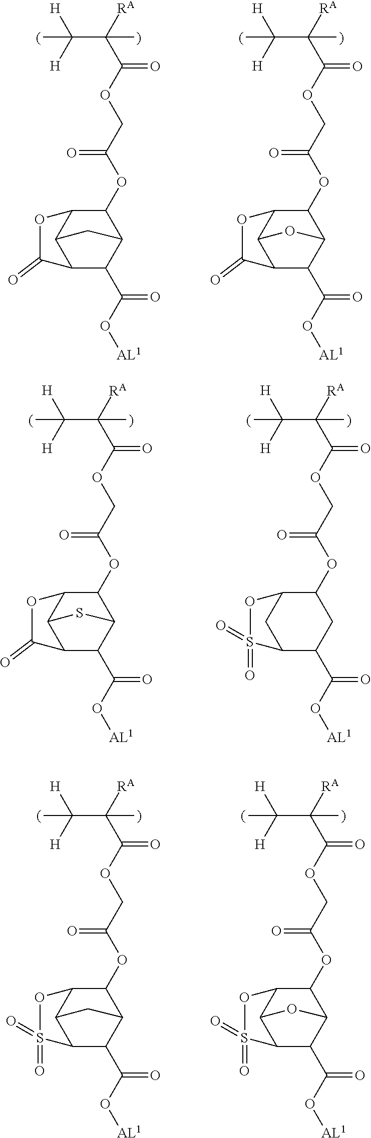

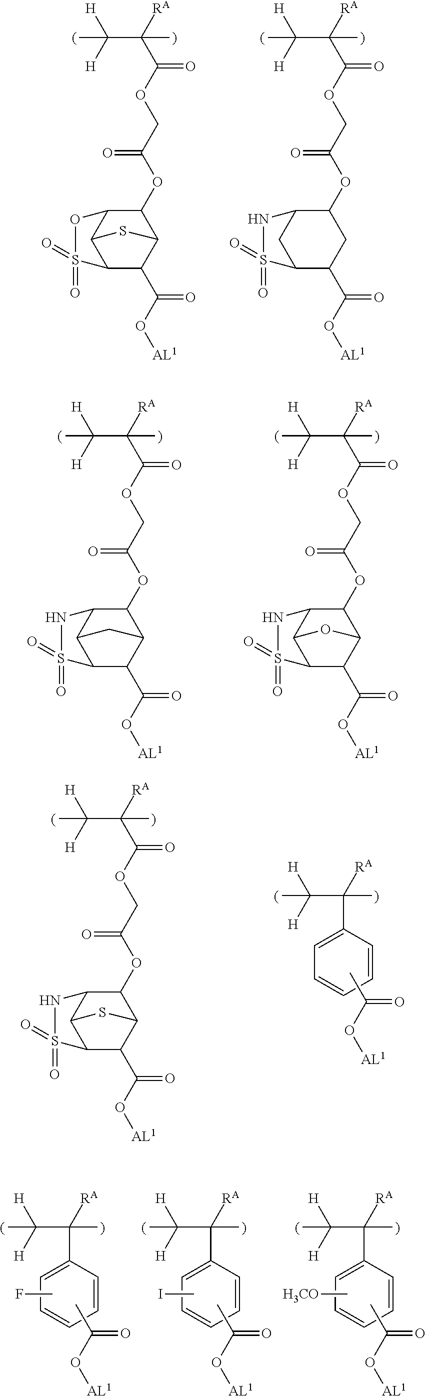

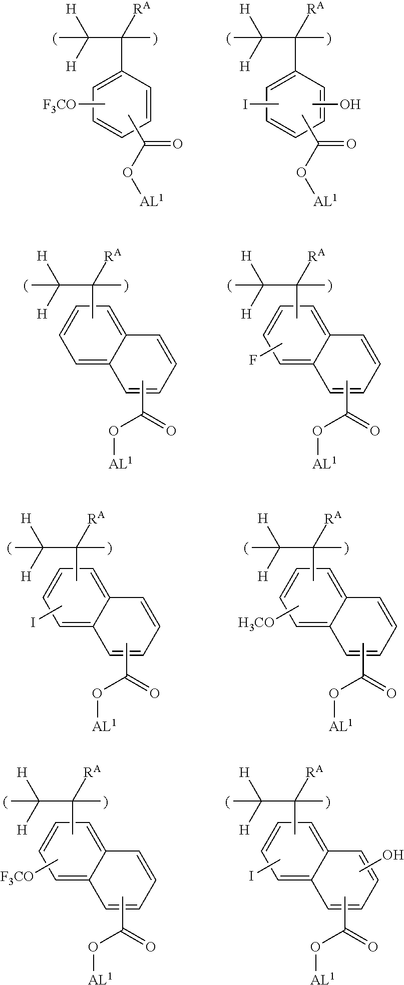

Examples of the repeat units (a2) include those shown below, but not limited thereto. In the formulae, R^{A} and AL² are as defined above.

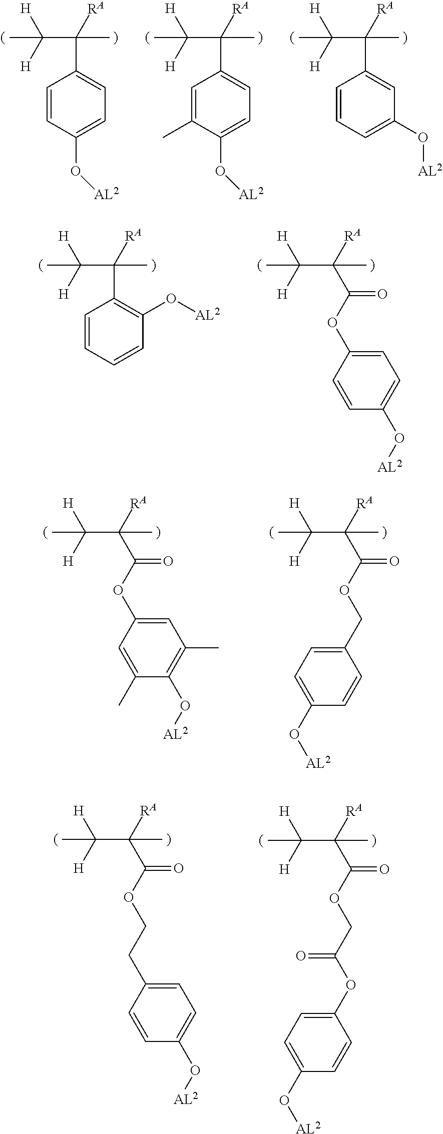

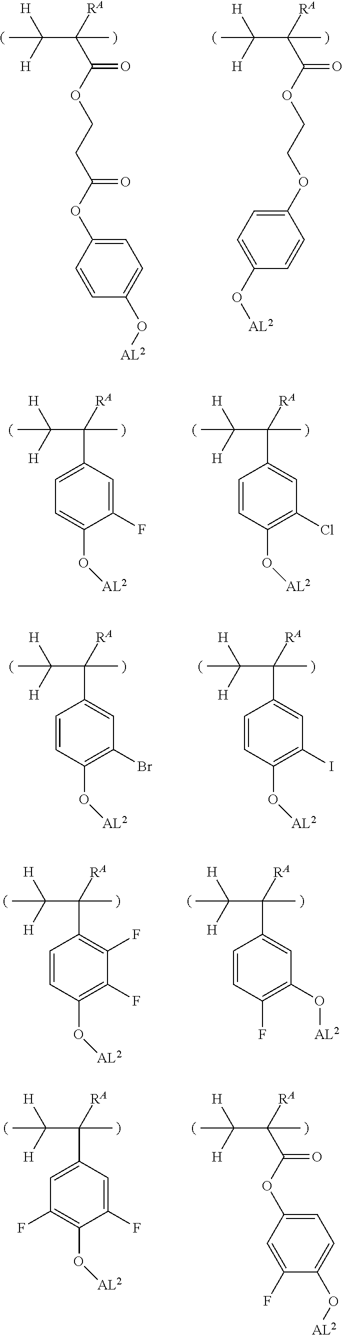

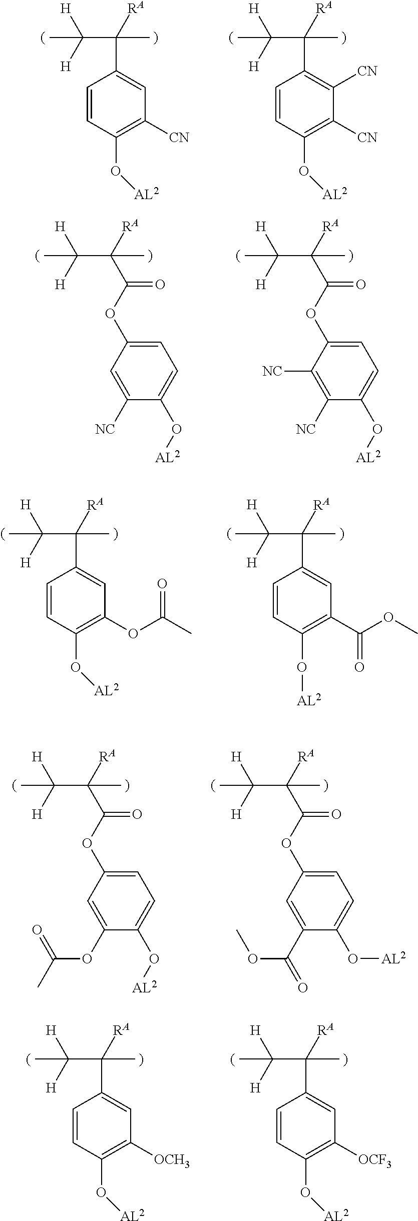

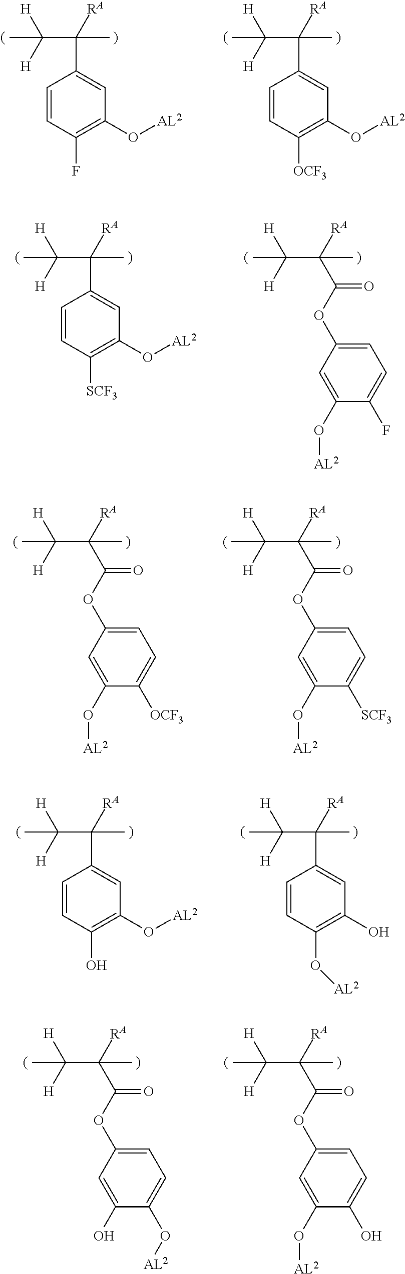

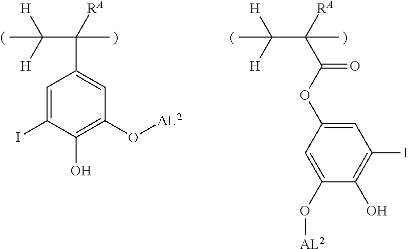

The polymer may comprise repeat units having the formula (a3), which are also referred to as repeat units (a3).

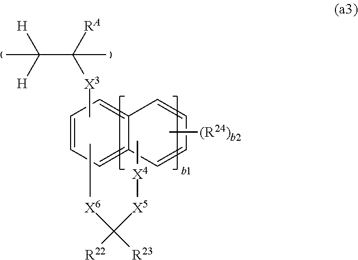

In the formula (a3), b1 is 0 or 1. The relevant structure is a benzene ring in case of b1=0, and a naphthalene ring in case of b1=1. The benzene ring corresponding to b1=0 is preferred from the aspect of solvent solubility. b2 is 0, 1, 2, or 3 in case of b1=0, and is 0, 1, 2, 3, 4, or 5 in case of b1=1. b2 is preferably 0, 1, 2, or 3, more preferably 0, 1, or 2 from the aspect of availability of reactants.

In the formula (a3), R^{A} is hydrogen, fluorine, methyl or trifluoromethyl. Among them, R^{A} is preferably hydrogen or methyl, more preferably hydrogen.

In the formula (a3), X³ is a single bond, *—C(═O)—O—, or *—C(═O)—NH—. The asterisk (*) designates a point of attachment to the carbon atom in the backbone. Among them, X³ is preferably a single bond or *—C(═O)—O—, more preferably a single bond.

In the formula (a3), X⁴ is a single bond, a C₁-C₄ aliphatic hydrocarbylene group, carbonyl group, sulfonyl group, or a group obtained by combining the foregoing. Of these, X⁴ is preferably a single bond, carbonyl group or sulfonyl group from the aspect of availability of reactants, more preferably a single bond or a carbonyl group from the aspect of a polar group created after reaction.

In the formula (a3), X⁵ and X⁶ are each independently oxygen or sulfur. Notably, X⁴ and X⁶ are bonded to vicinal carbon atoms on the aromatic ring. X⁵ and X⁶ may be identical or different. Both X⁵ and X⁶ are preferably oxygen from the aspect of reactivity.

In the formula (a3), R²² and R²³ are each independently hydrogen or a C₁-C₂₀ hydrocarbyl group which may contain a heteroatom. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, 1-propenyl, 2-propenyl, butenyl, and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl, and 2-phenylethyl; and combinations thereof. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety.

Also, R²² and R²³ may bond together to form a ring with the carbon atoms to which they are attached. Examples of the ring thus formed include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

In the formula (a3), R²⁴ is halogen, hydroxy, cyano, nitro, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{24A})(R^{24B}). R^{24A} and R^{24B} are each independently hydrogen or a C₁-C₆ hydrocarbyl group. The halogen atom is preferably fluorine, chlorine, bromine or iodine, more preferably fluorine or iodine. The hydrocarbyl group and hydrocarbyl moiety in the hydrocarbyloxy, hydrocarbyloxycarbonyl and hydrocarbylthio groups may be saturated or unsaturated and straight, branched or cyclic. Examples thereof include those exemplified above for the hydrocarbyl groups represented by R²² and R²³. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety. A plurality of R²⁴ may be identical or different when b2 is 2 or more.

Also, when b2 is 2 or more, a plurality of R²⁴ may bond together to form a ring with the carbon atom of the aromatic ring to which they are attached. Examples of the ring thus formed include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

Examples of the repeat units (a3) include those shown below, but not limited thereto. In the formulae. R^{A} is as defined above, and Me is methyl. The bond positions of substituents on the aromatic ring are interchangeable.

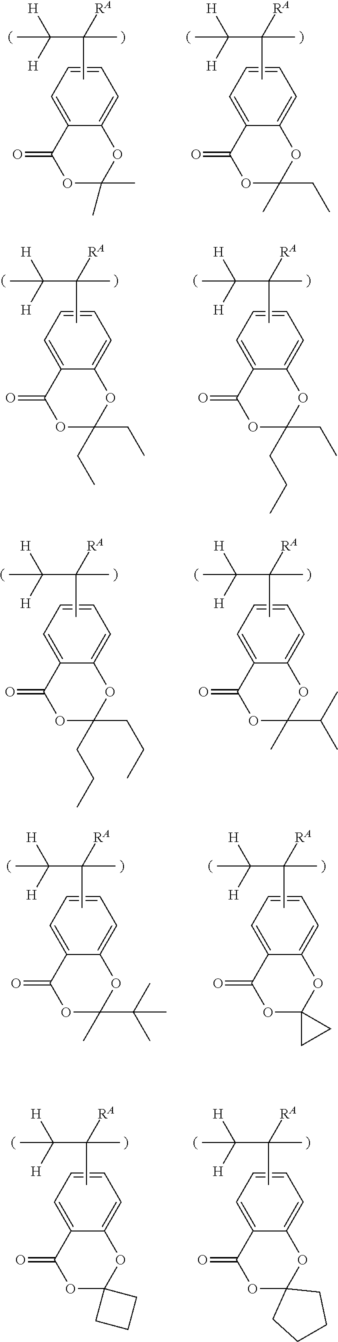

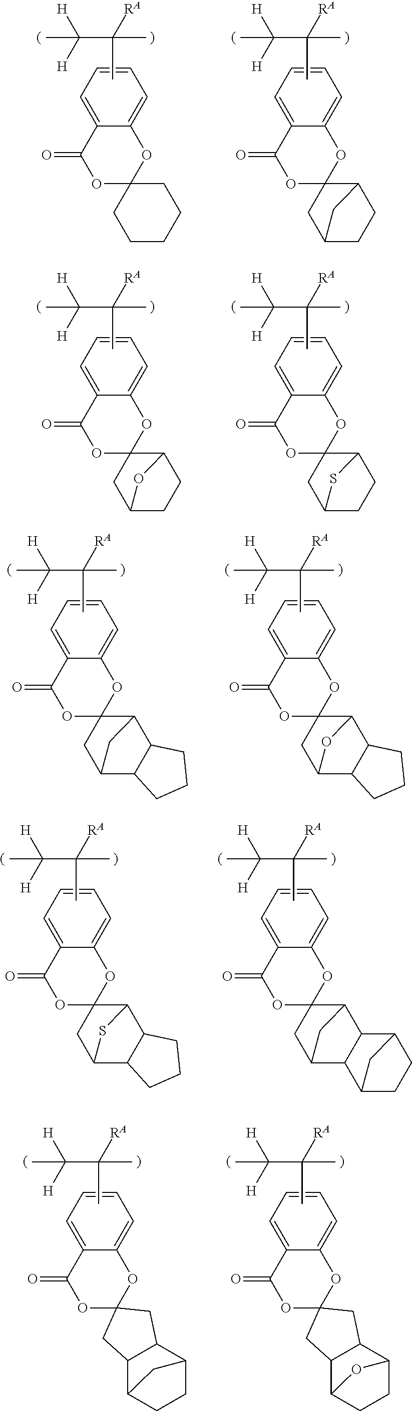

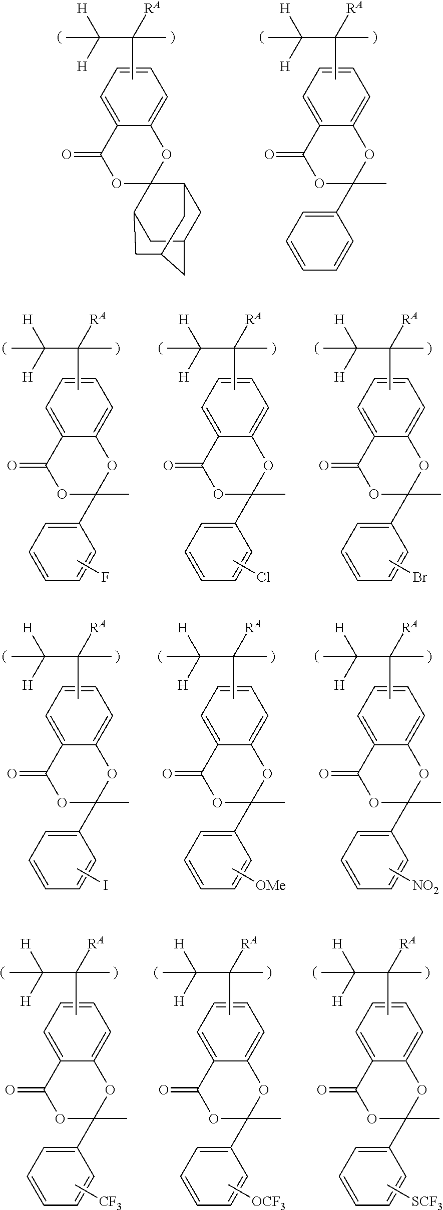

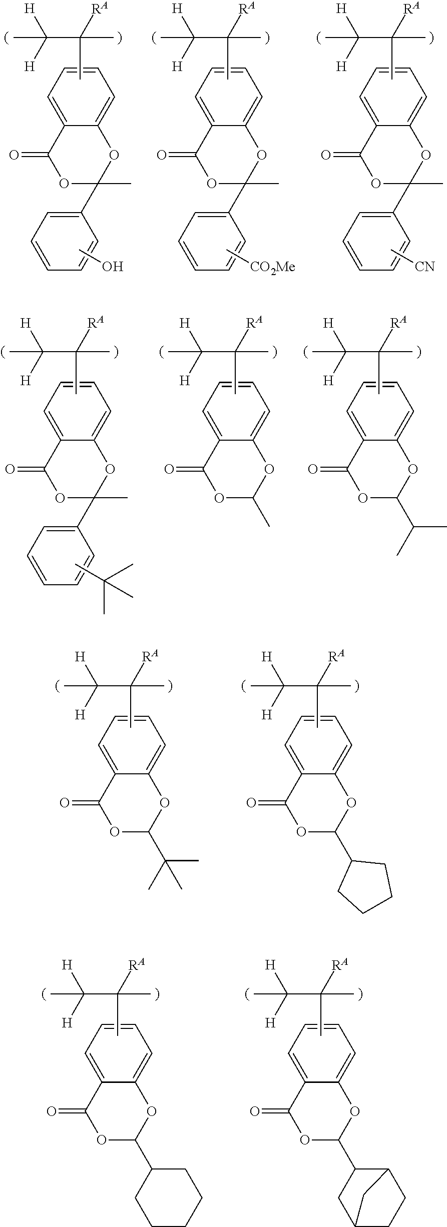

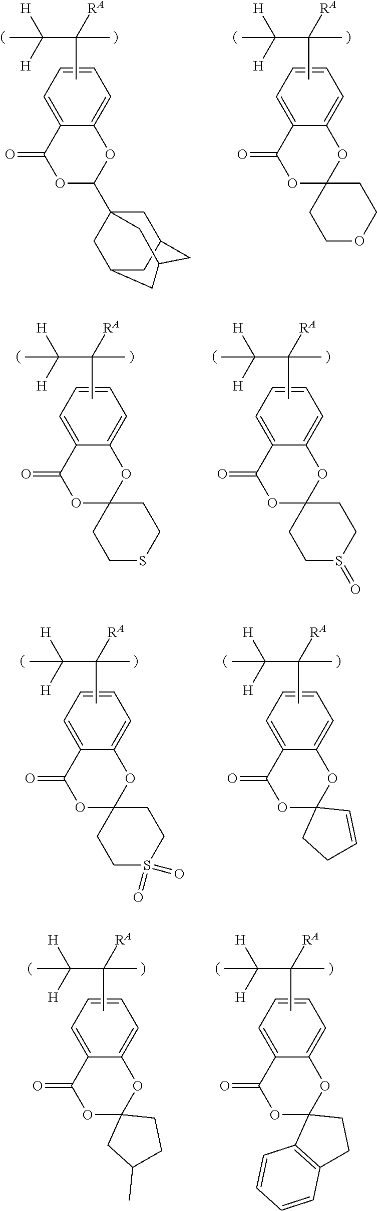

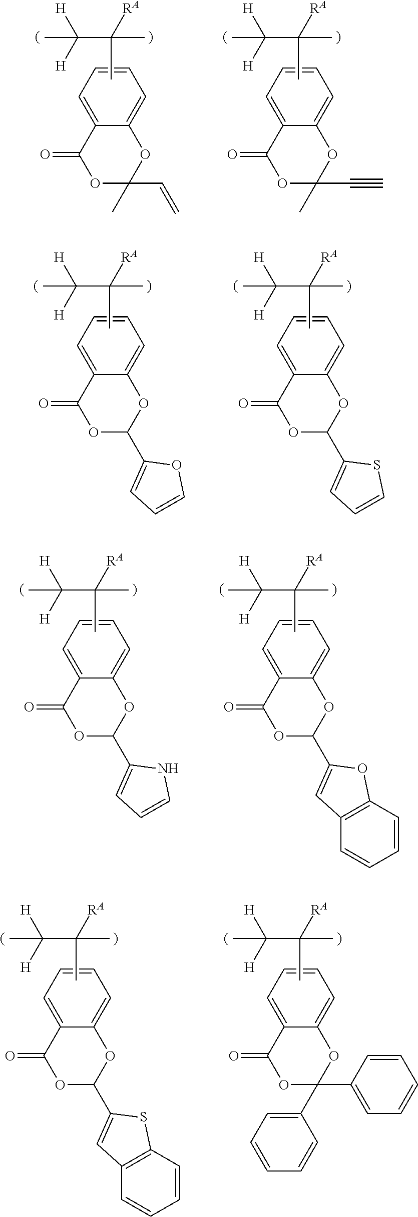

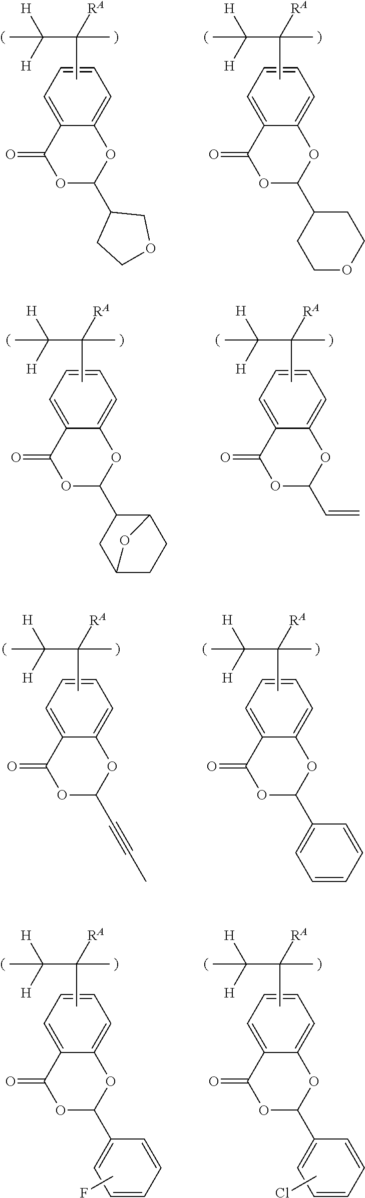

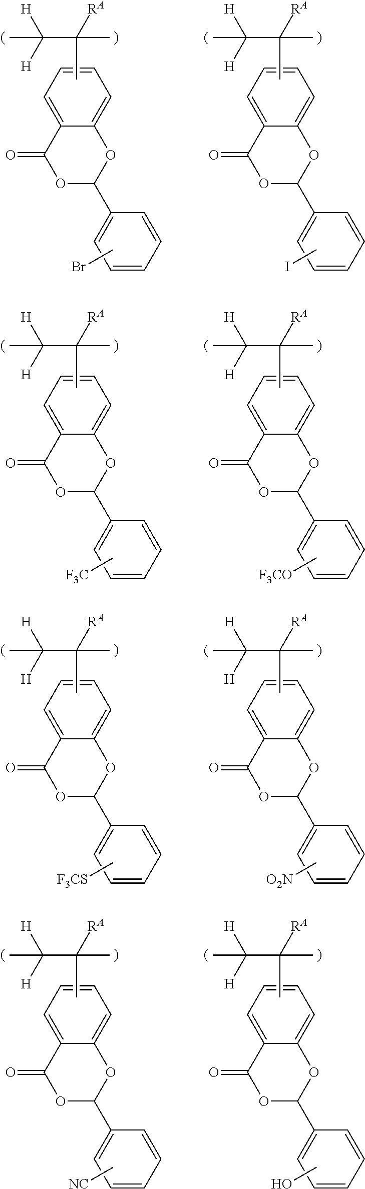

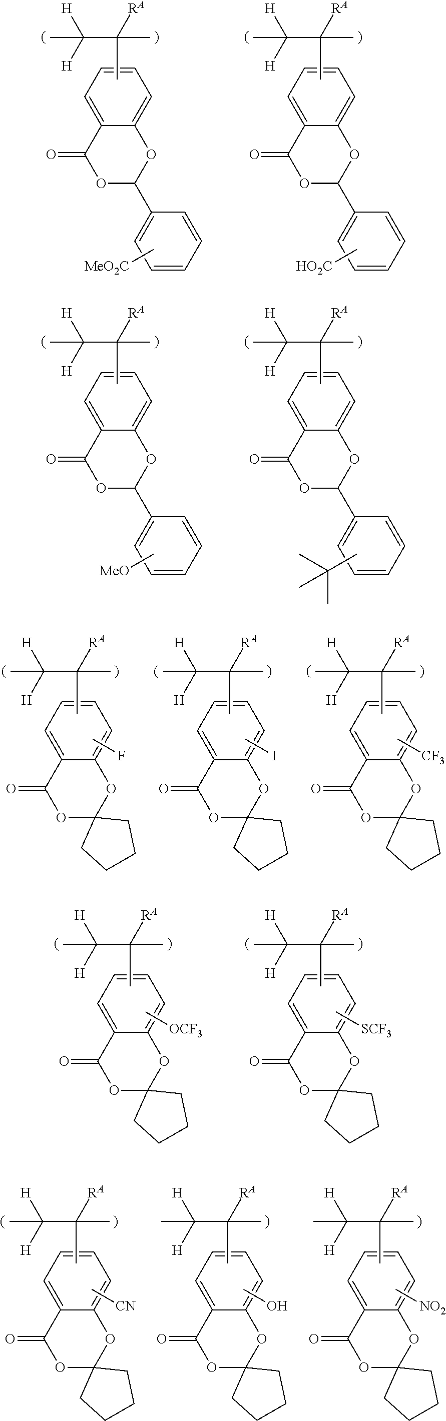

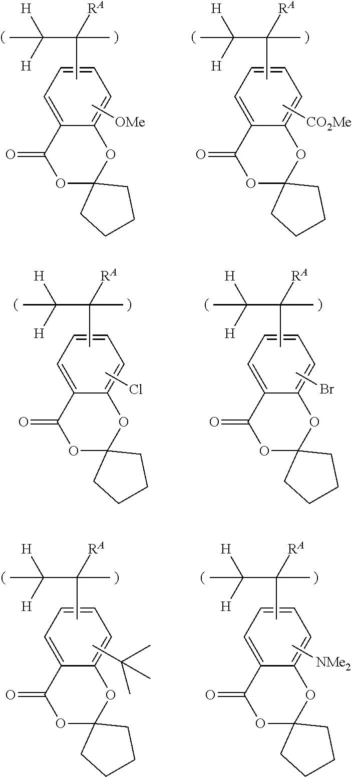

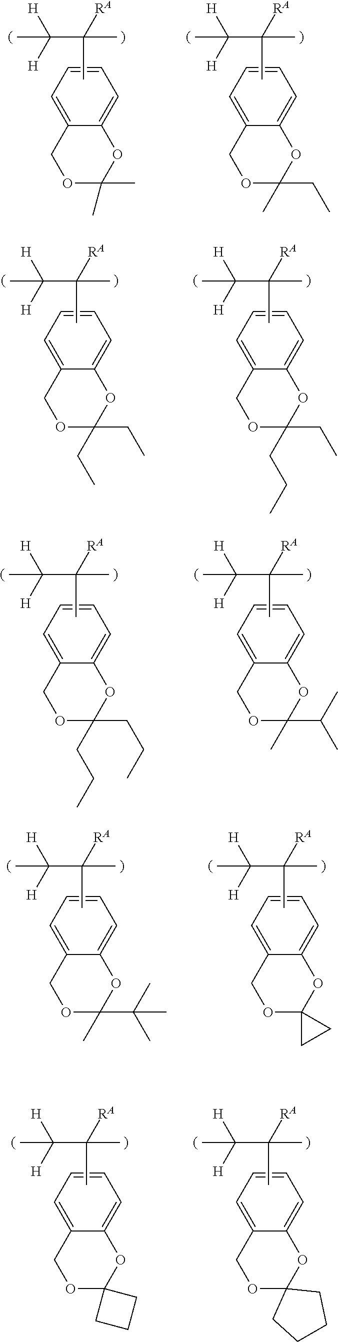

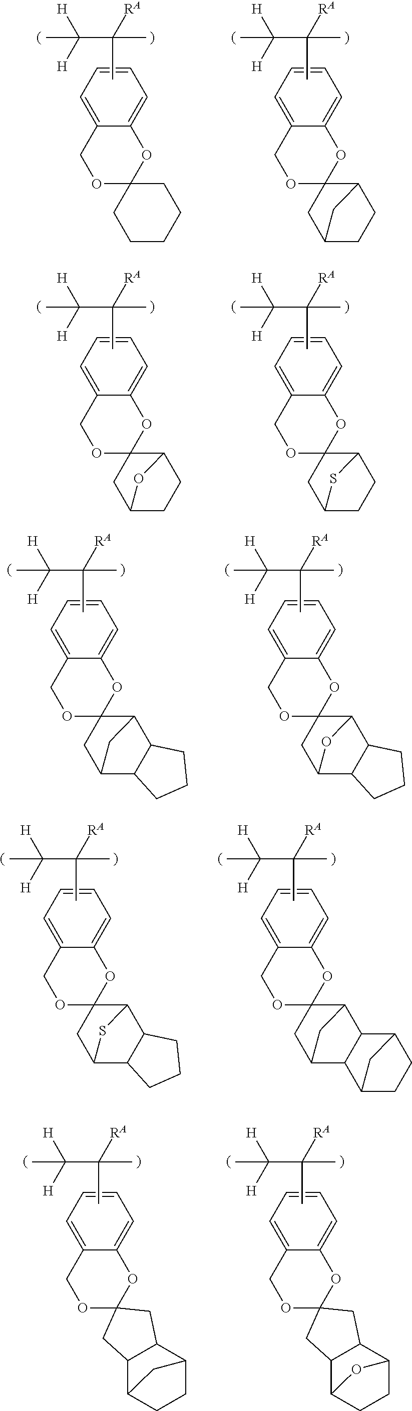

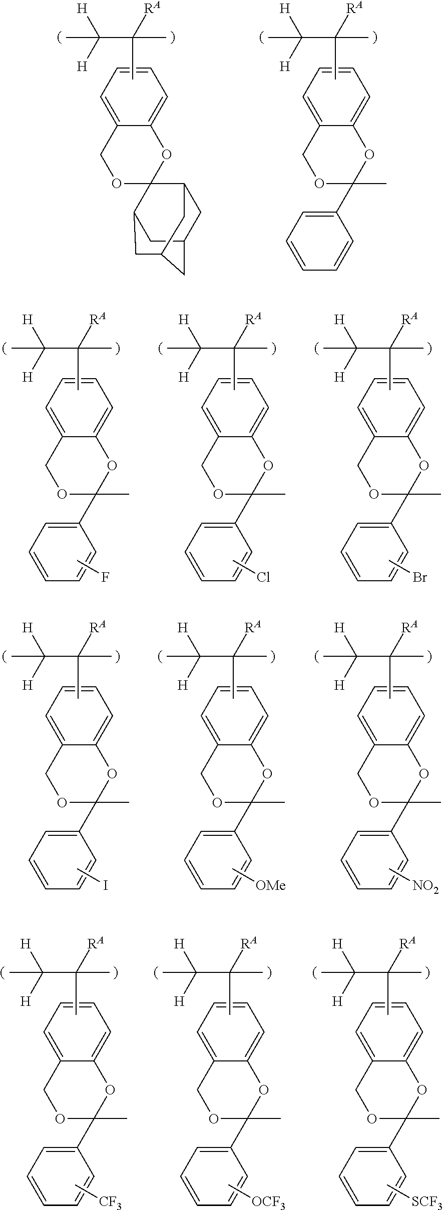

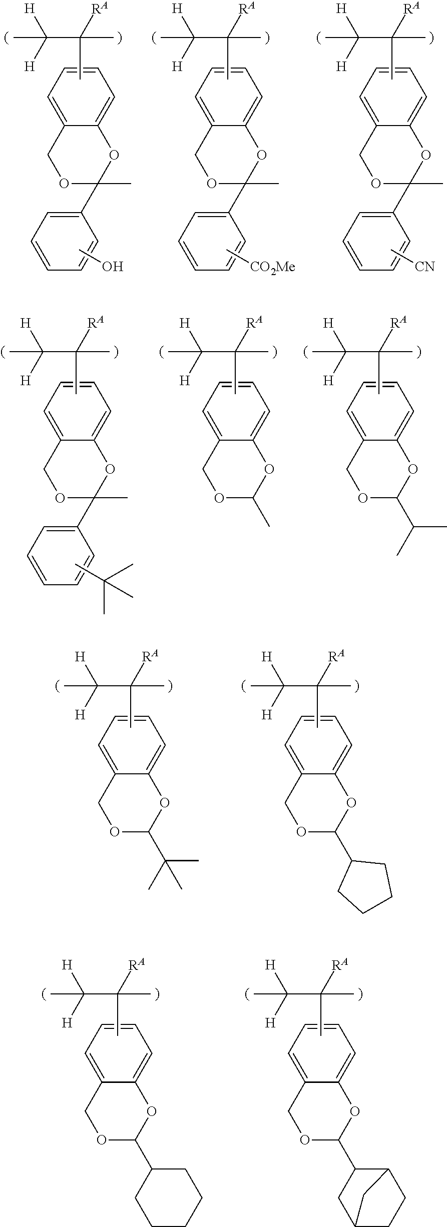

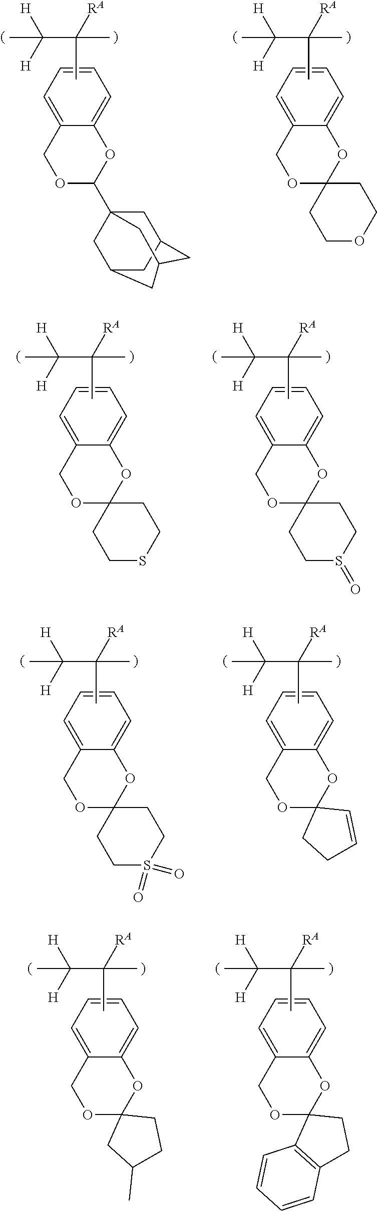

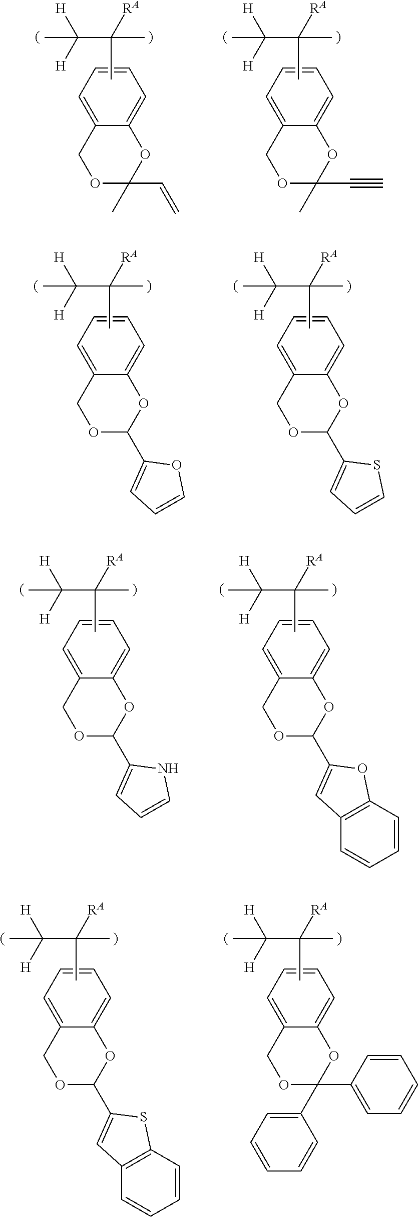

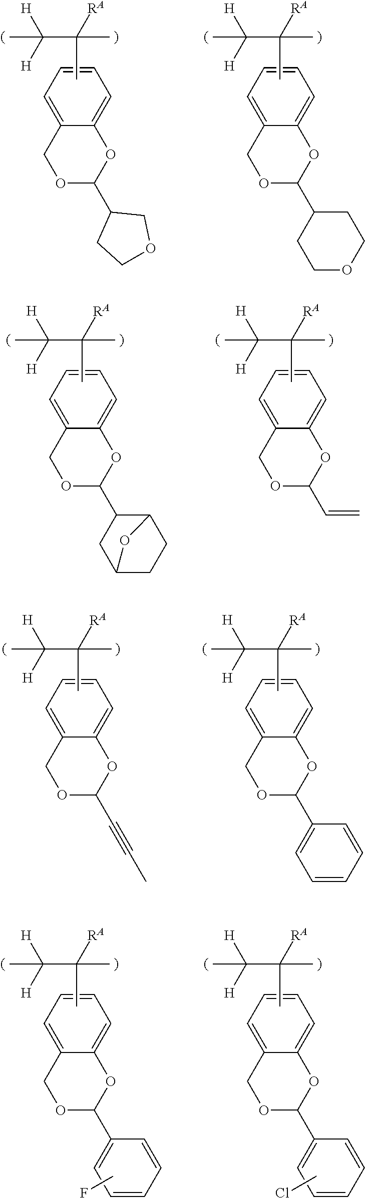

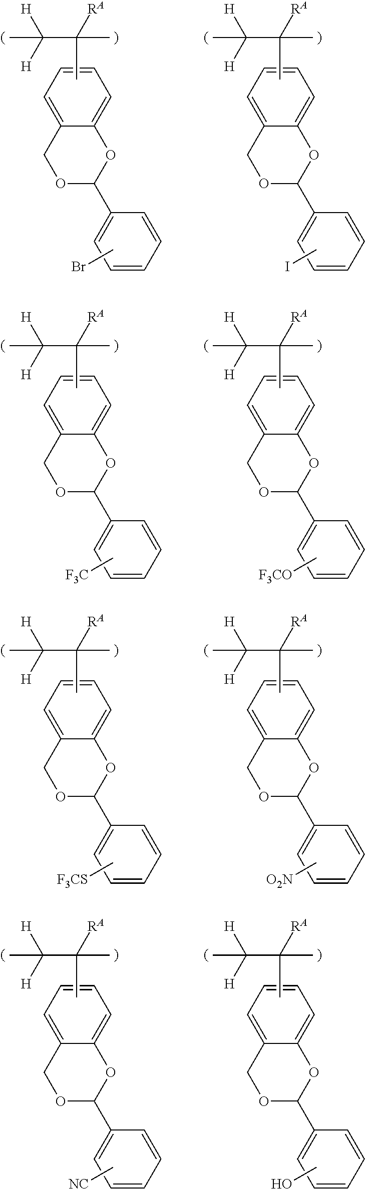

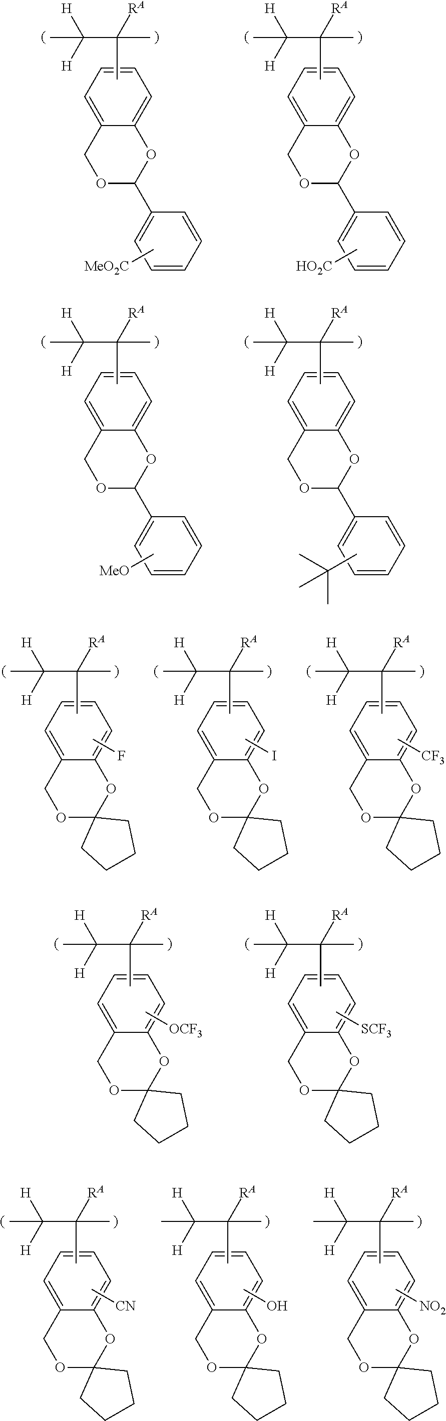

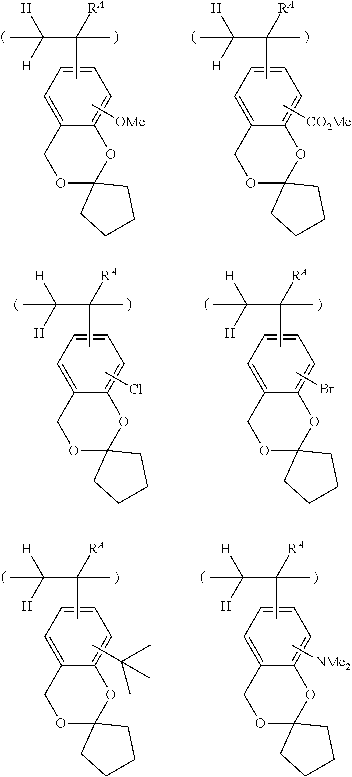

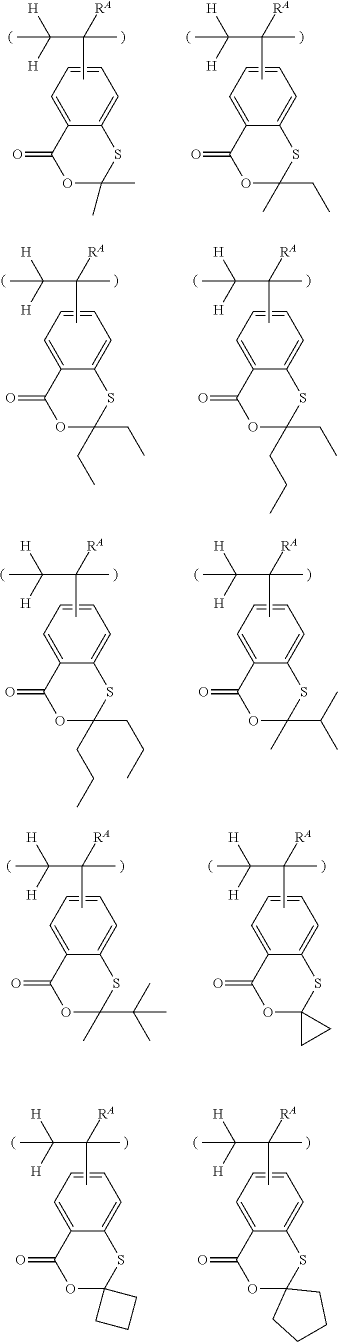

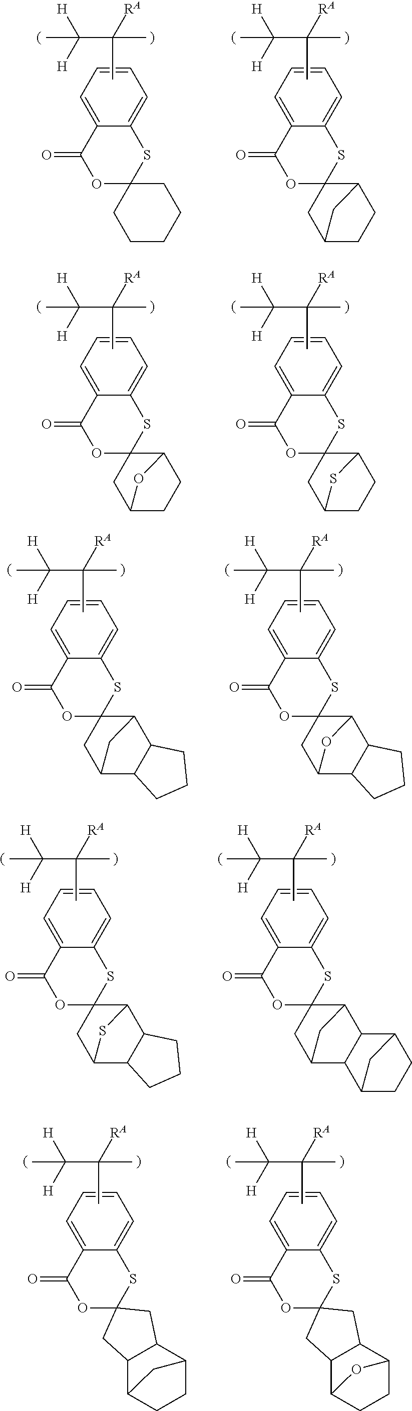

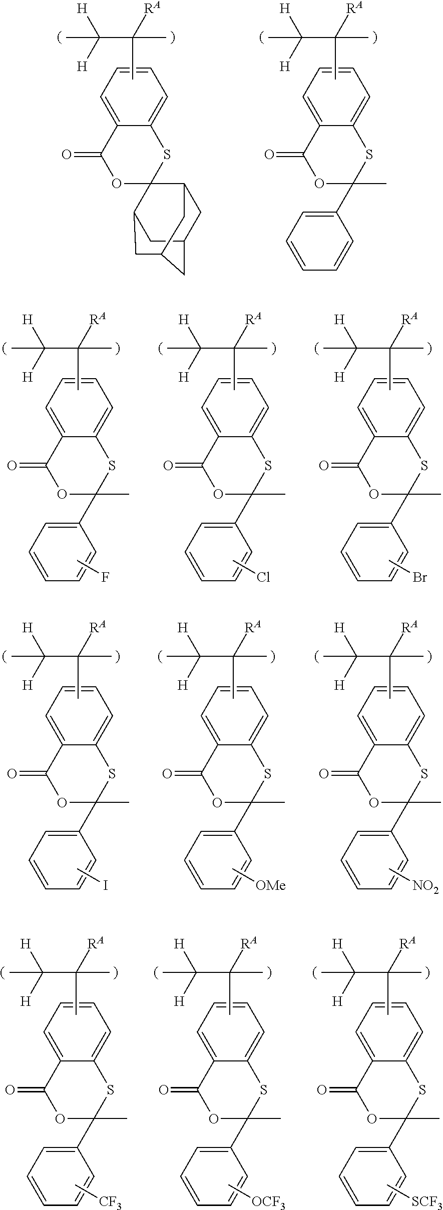

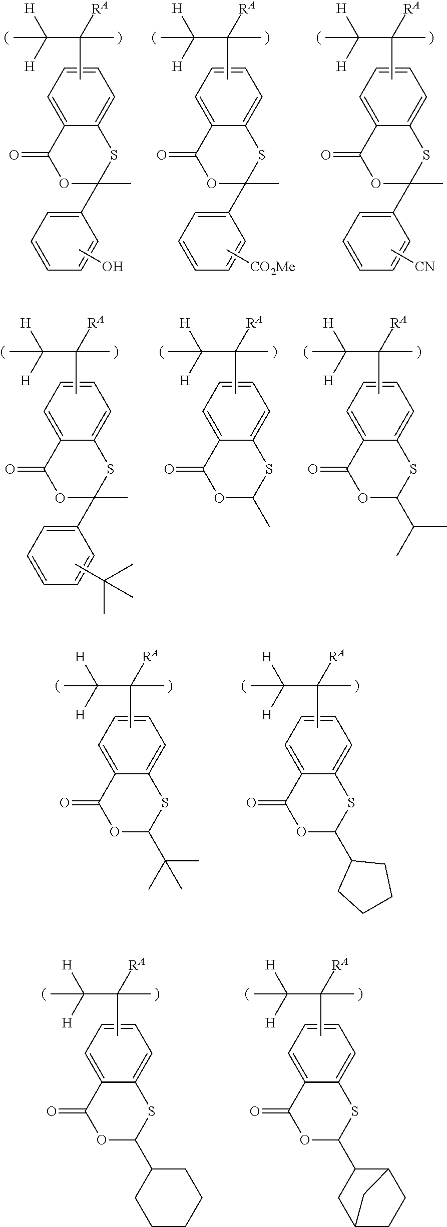

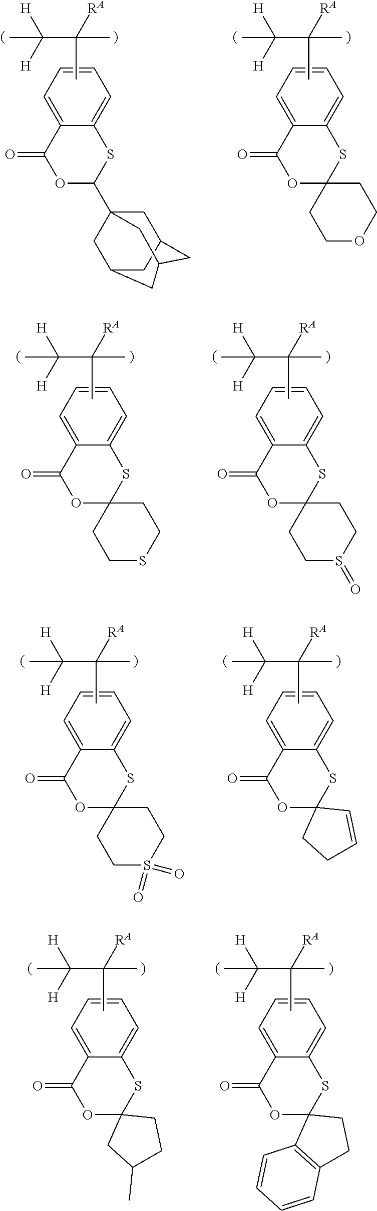

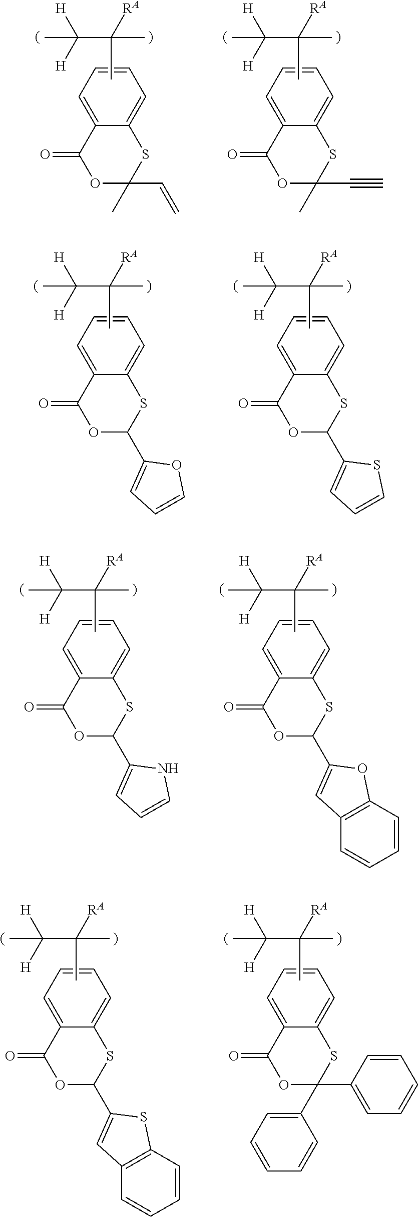

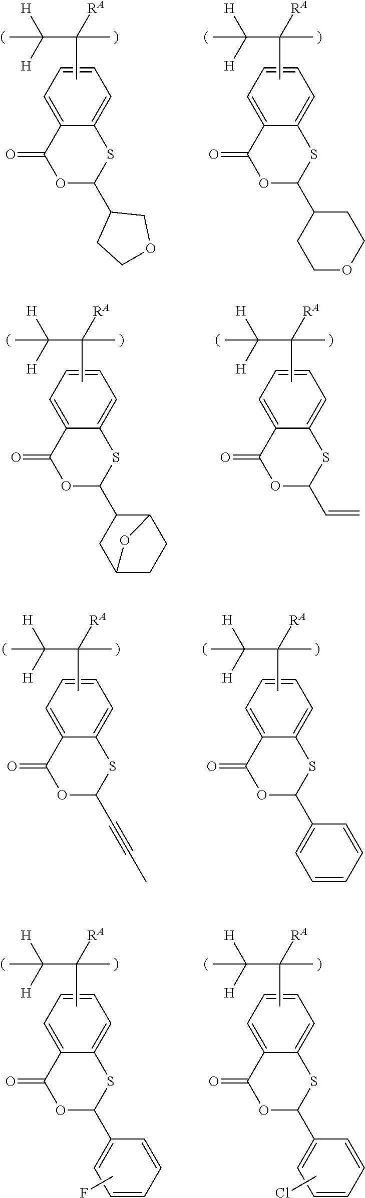

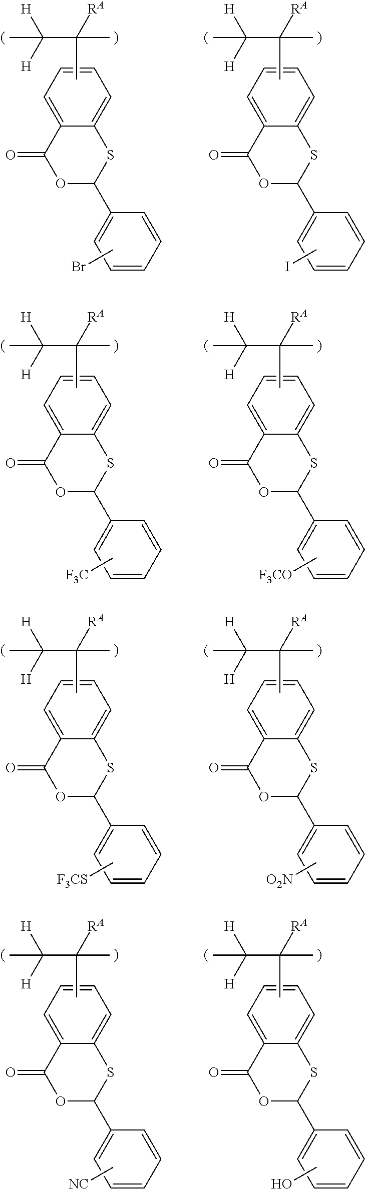

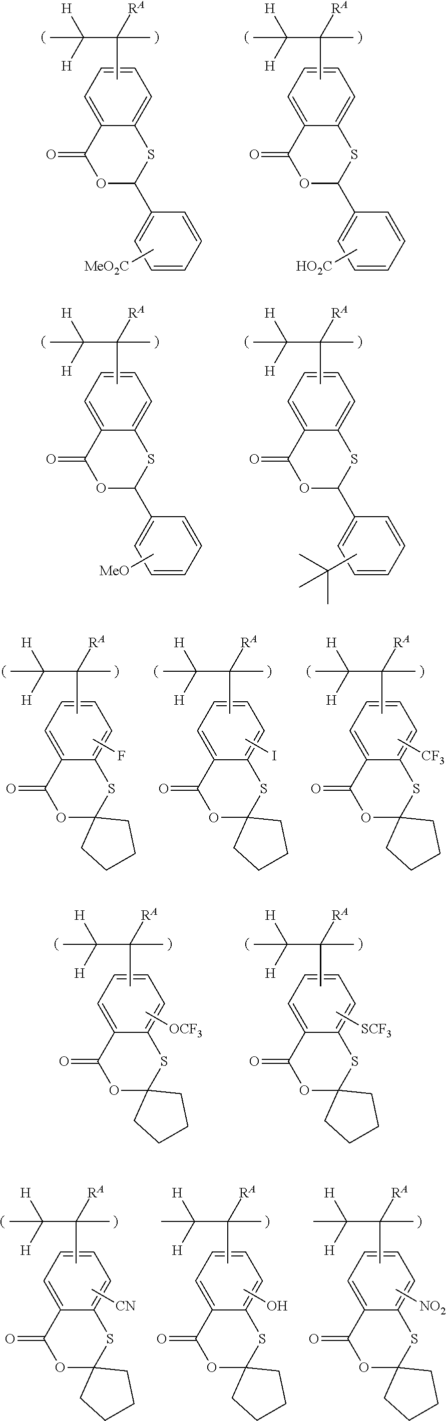

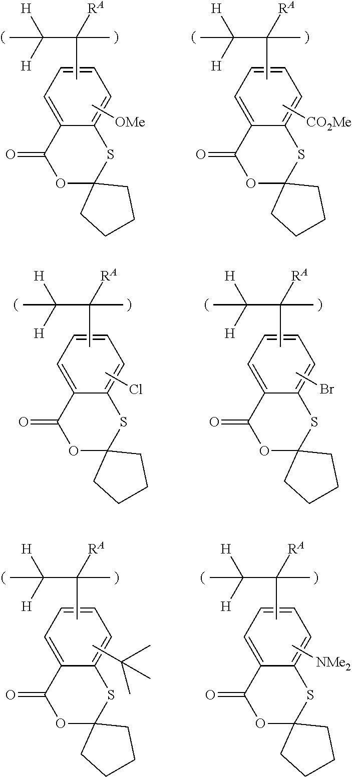

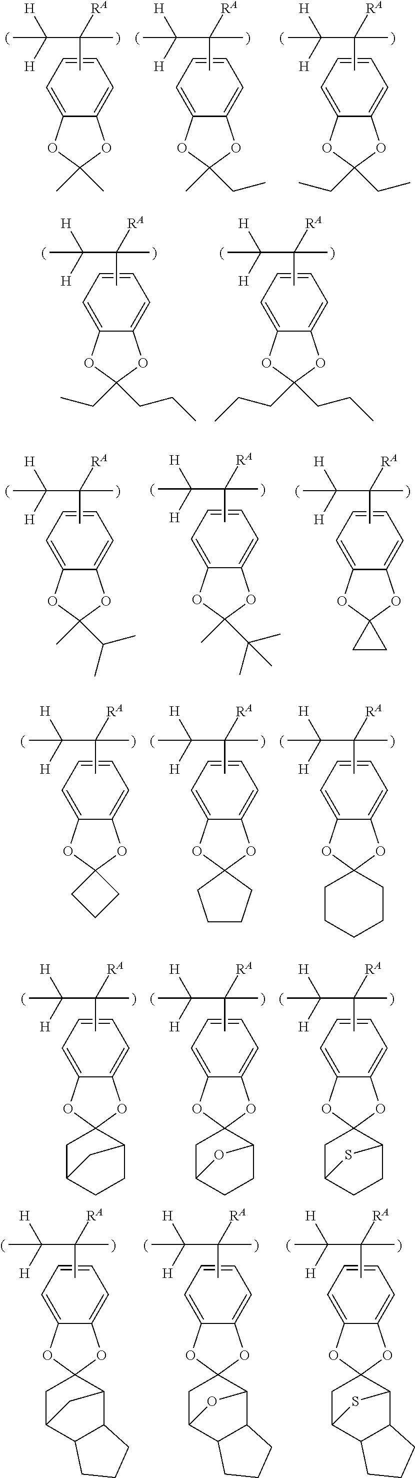

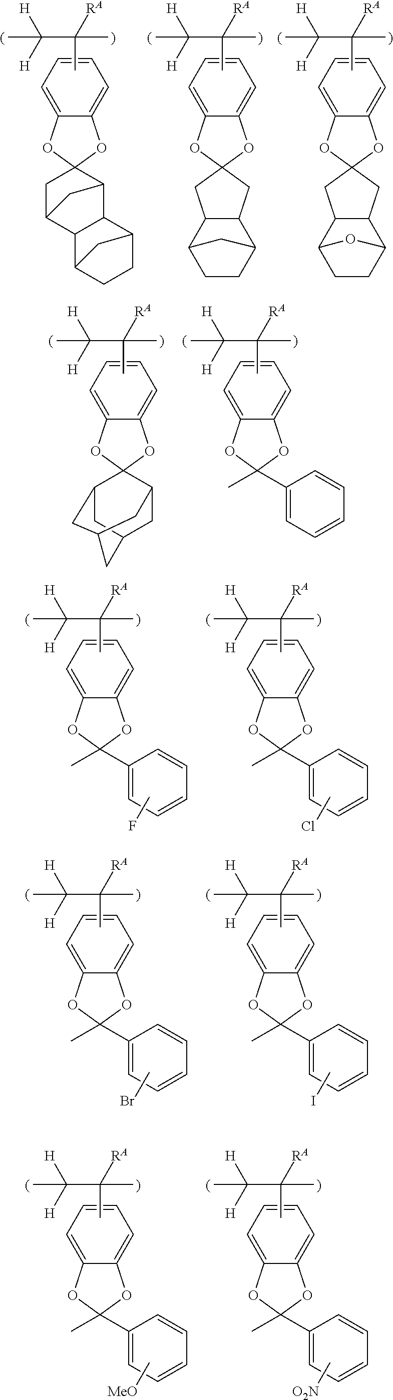

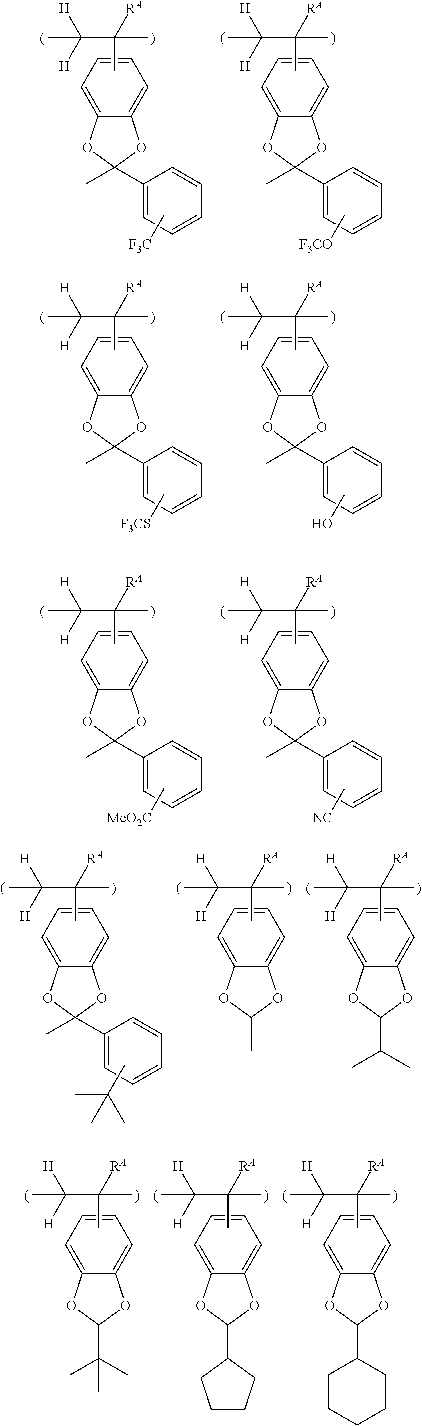

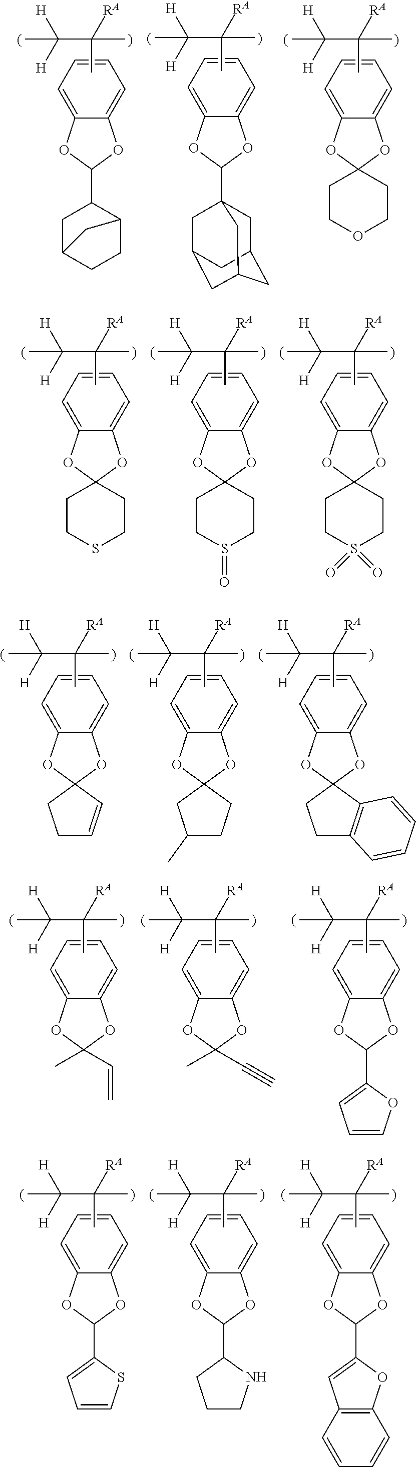

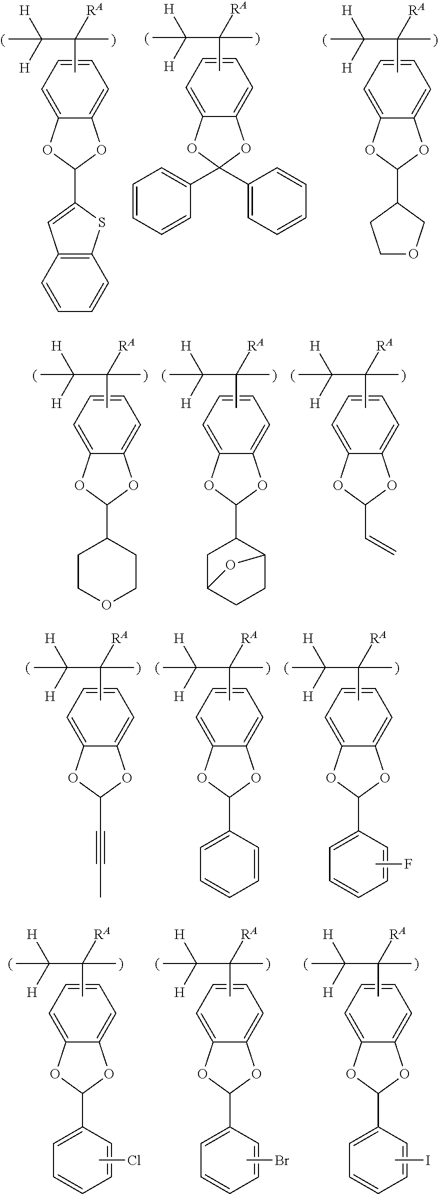

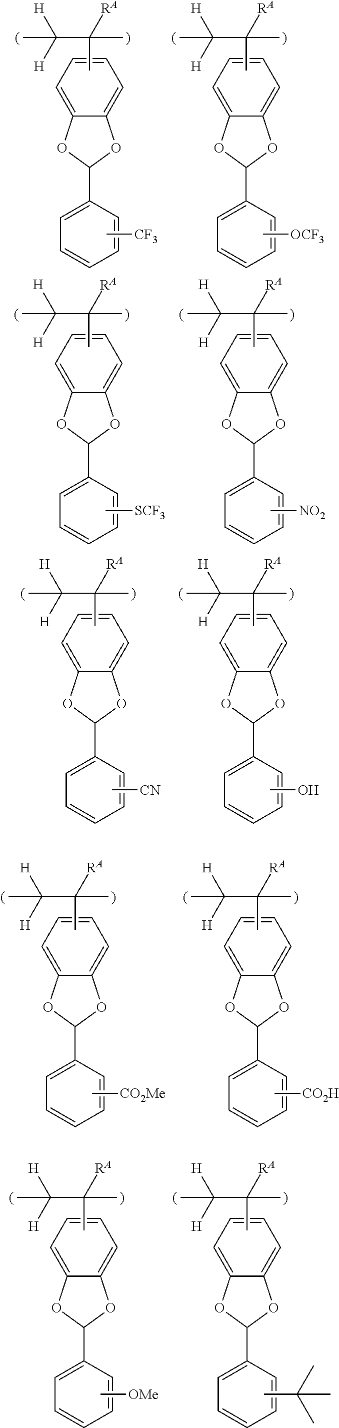

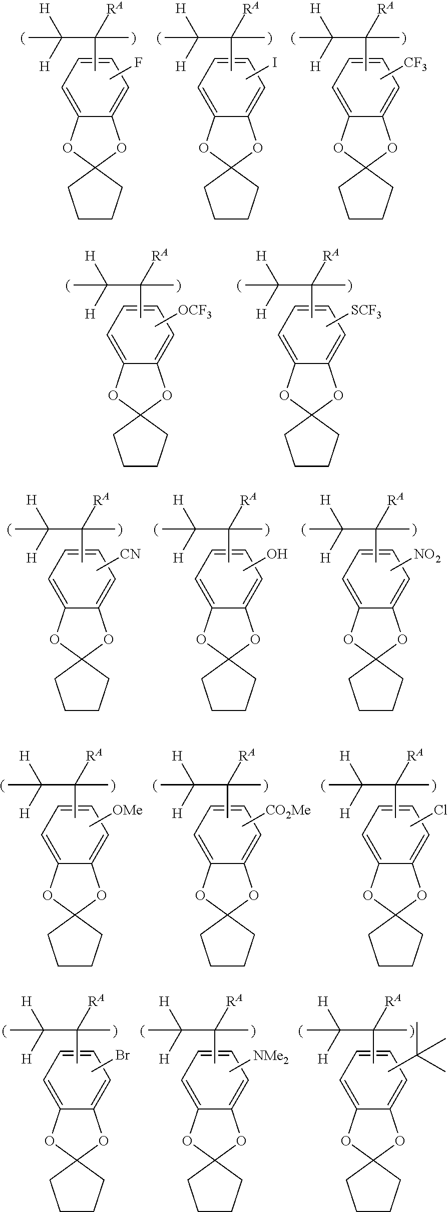

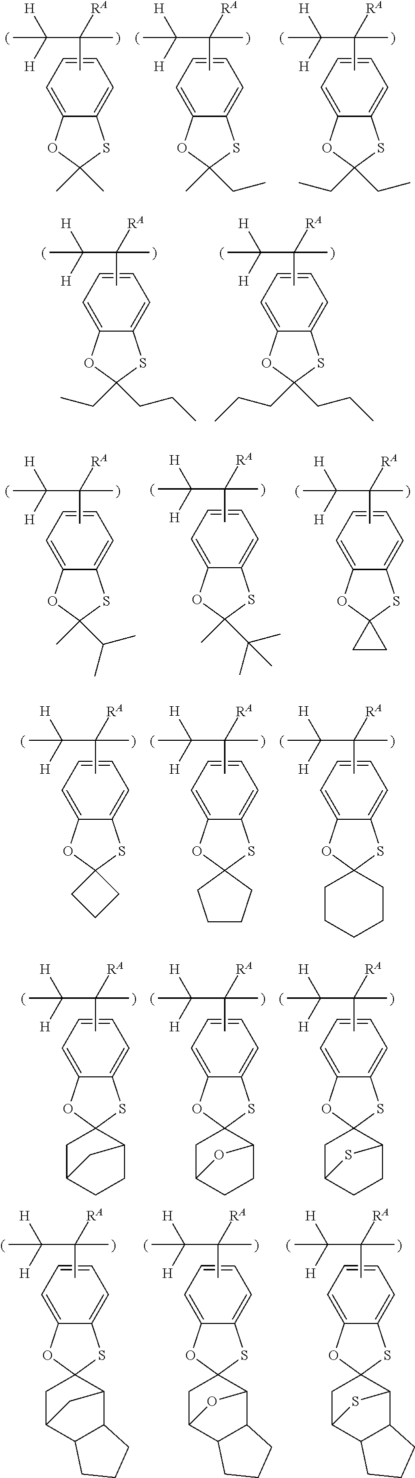

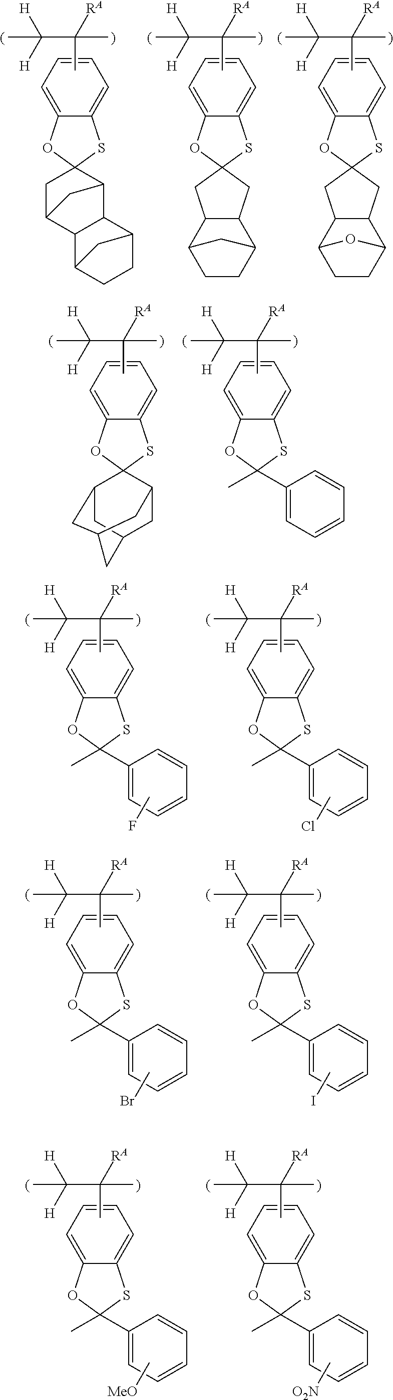

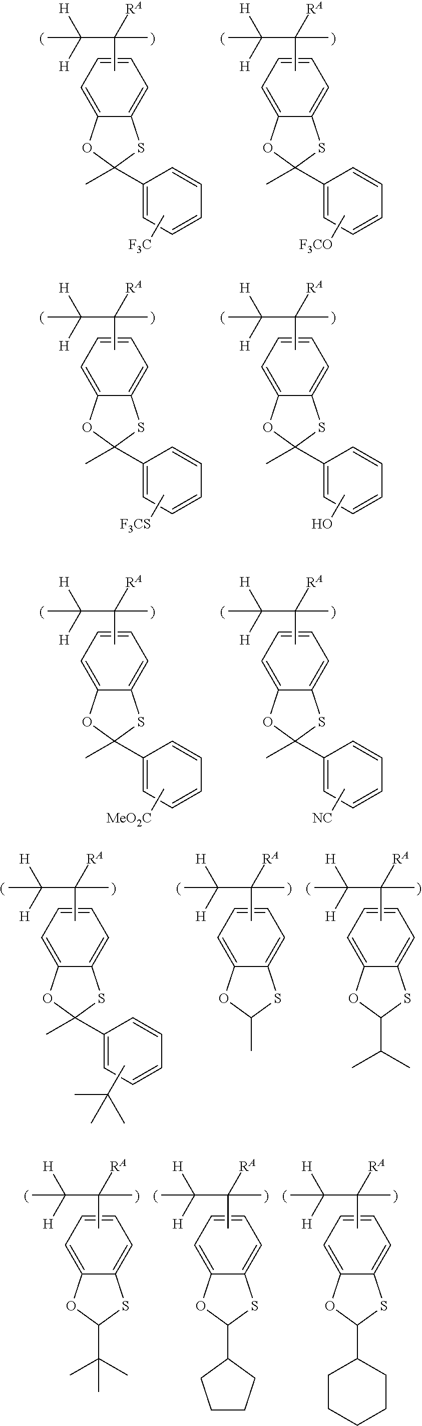

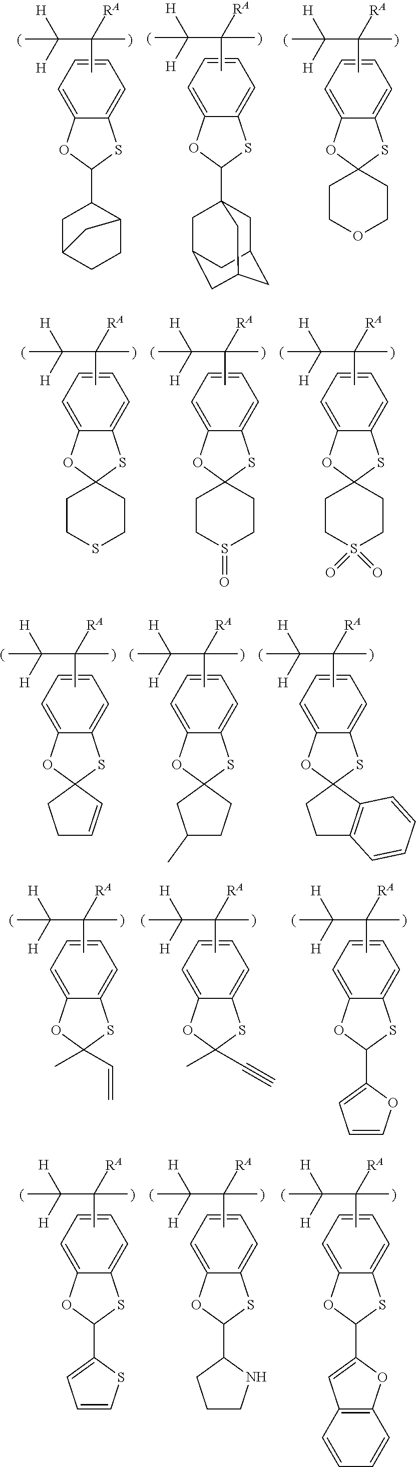

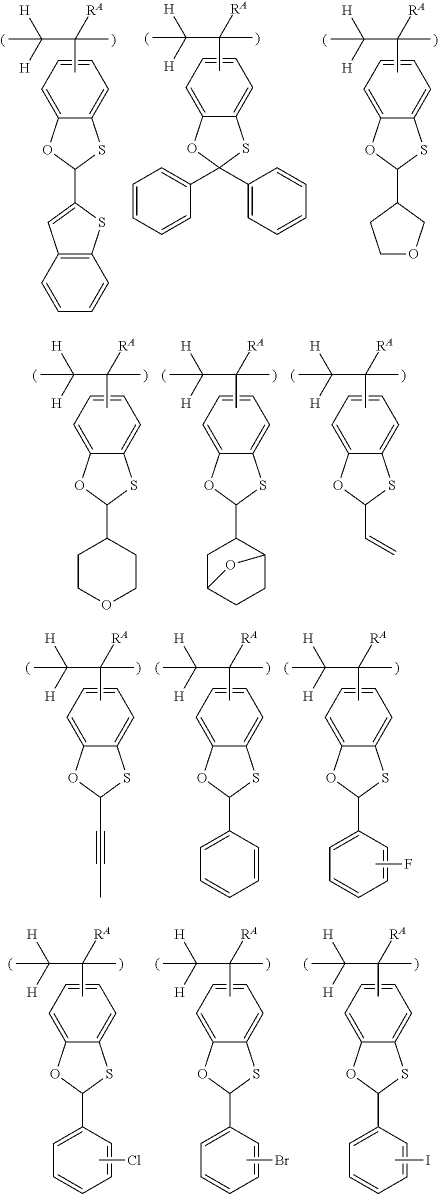

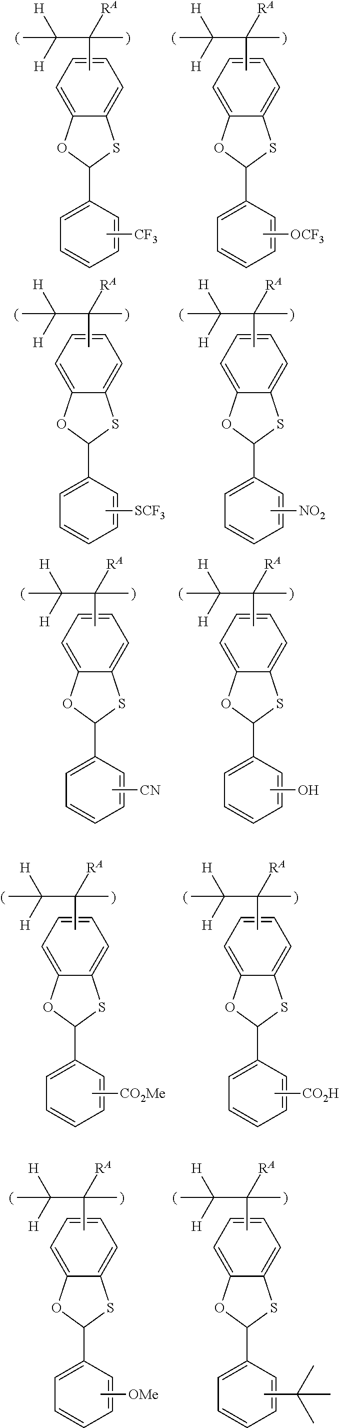

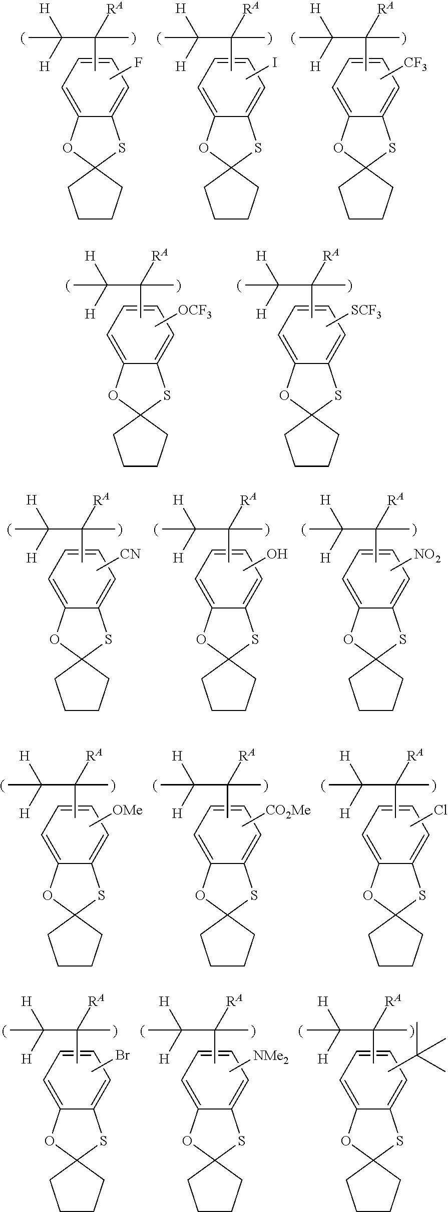

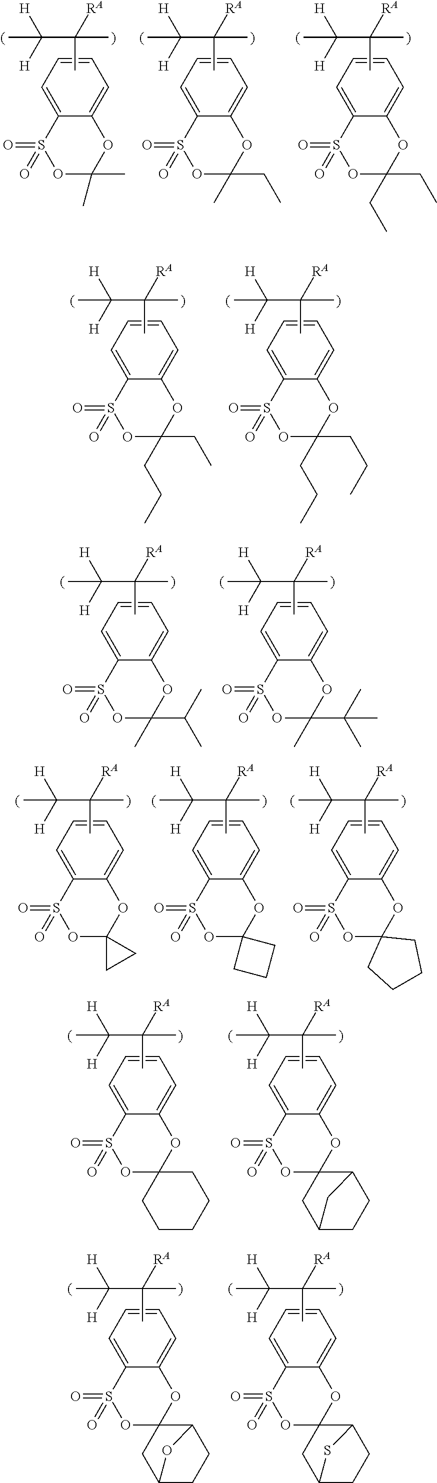

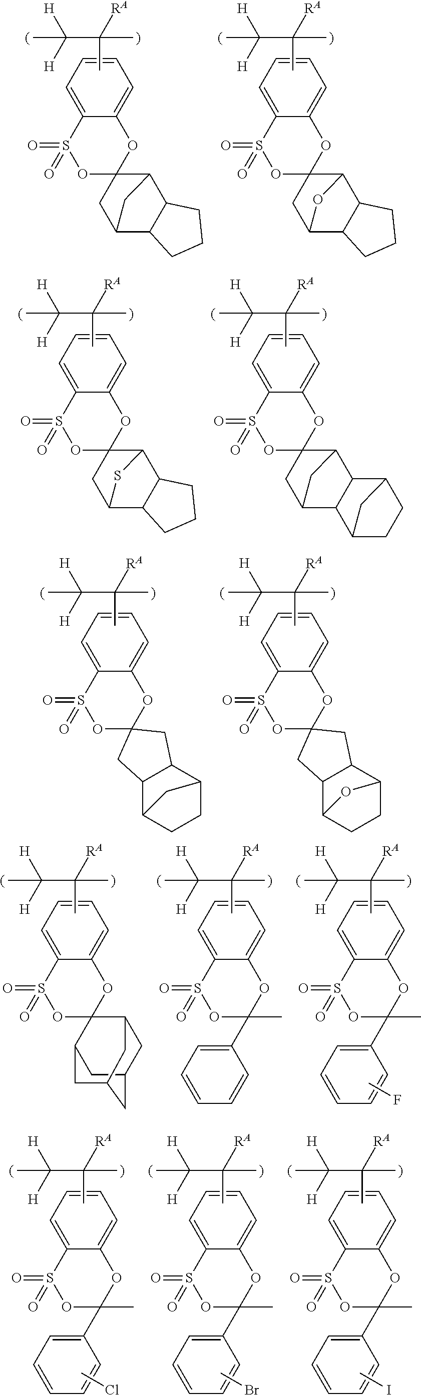

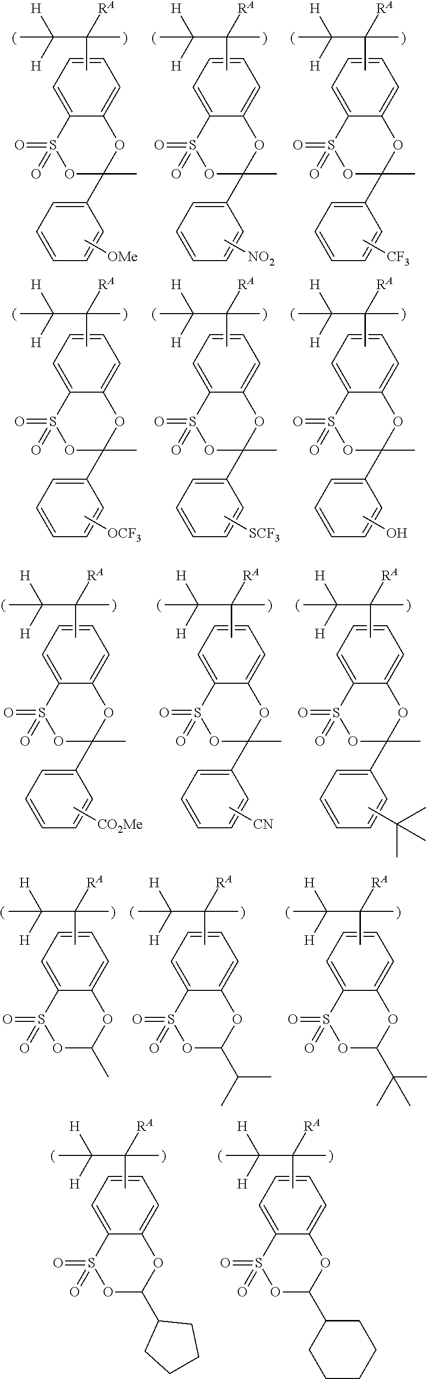

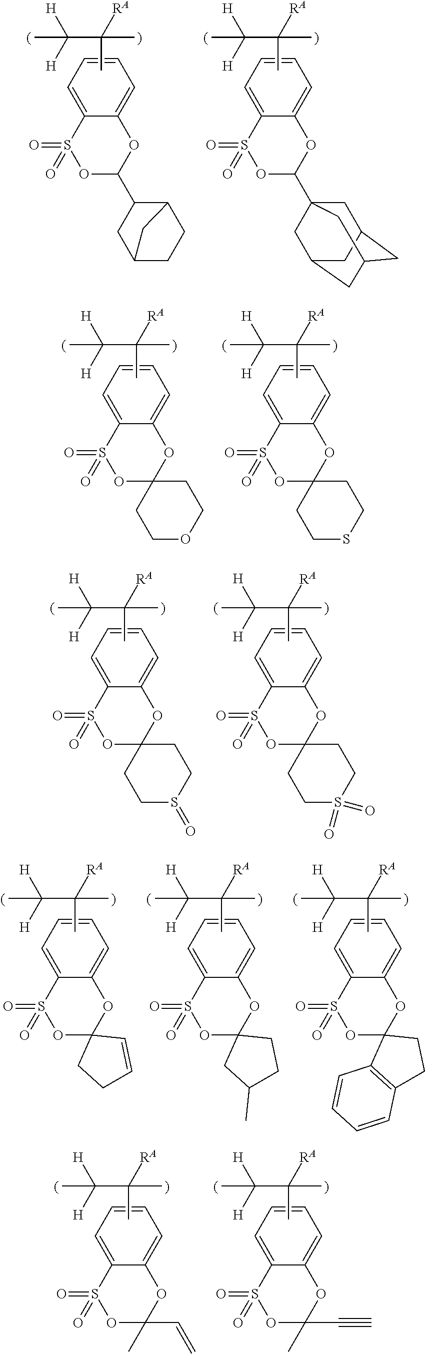

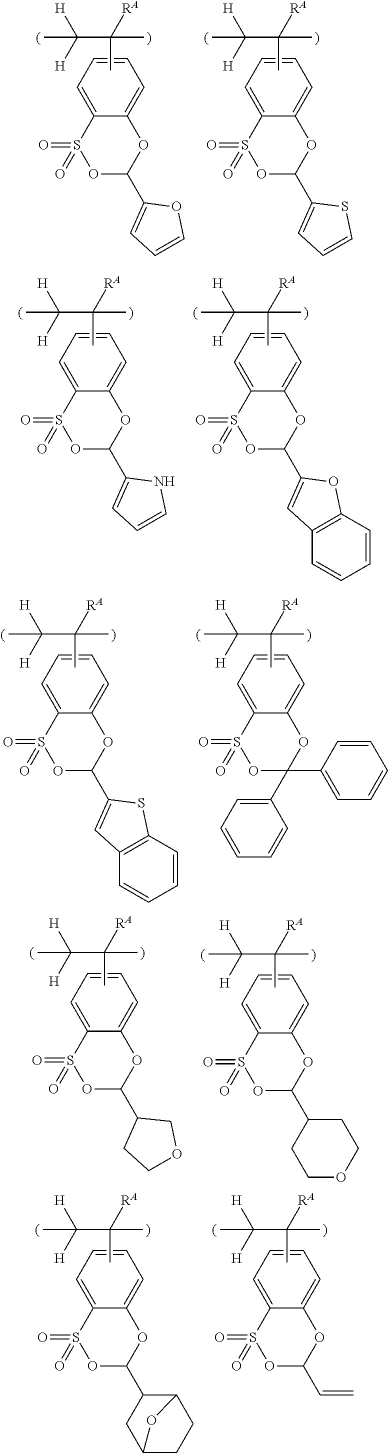

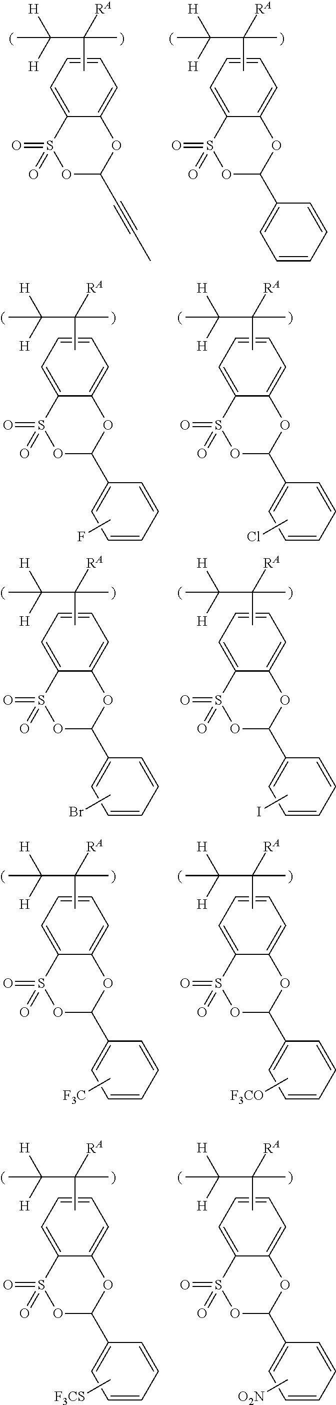

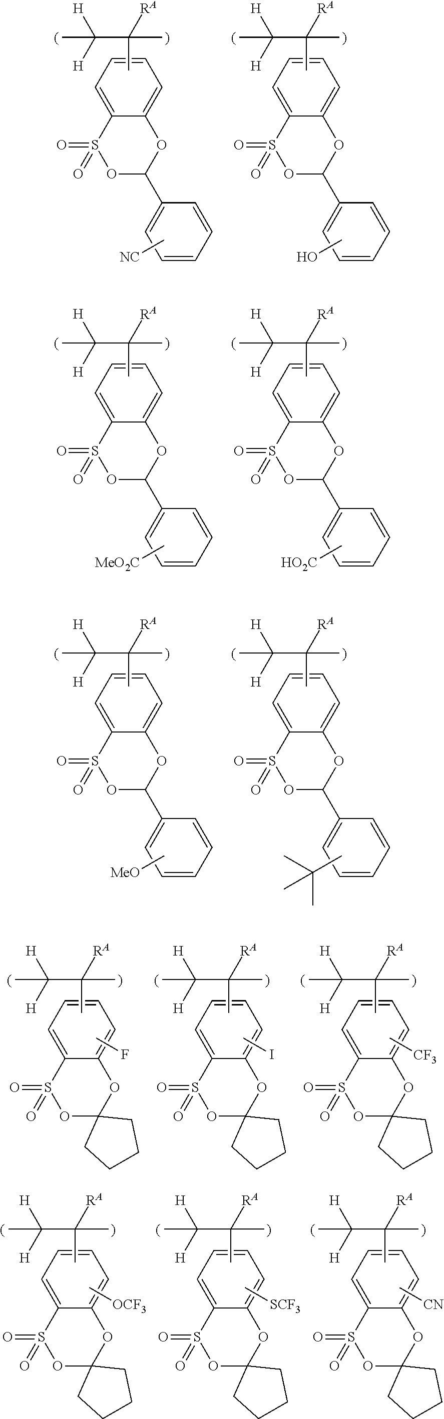

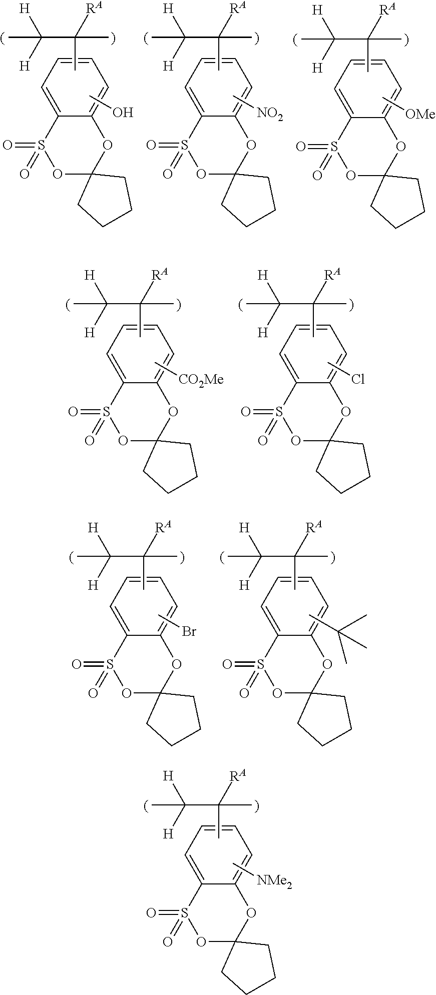

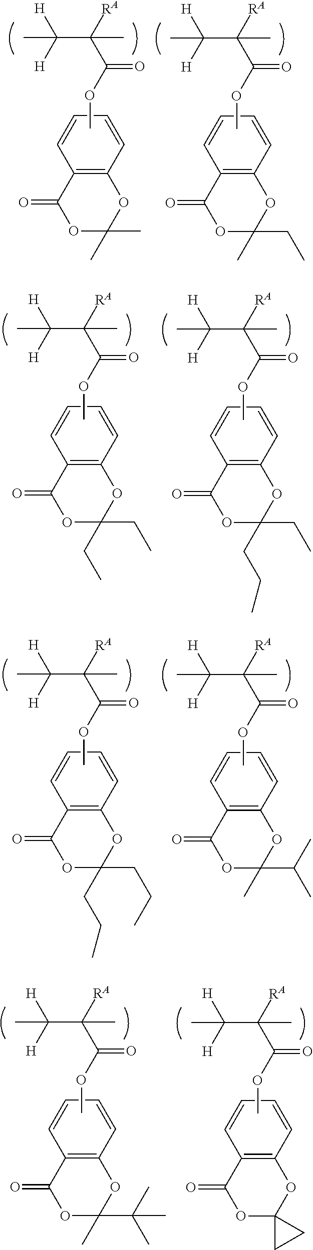

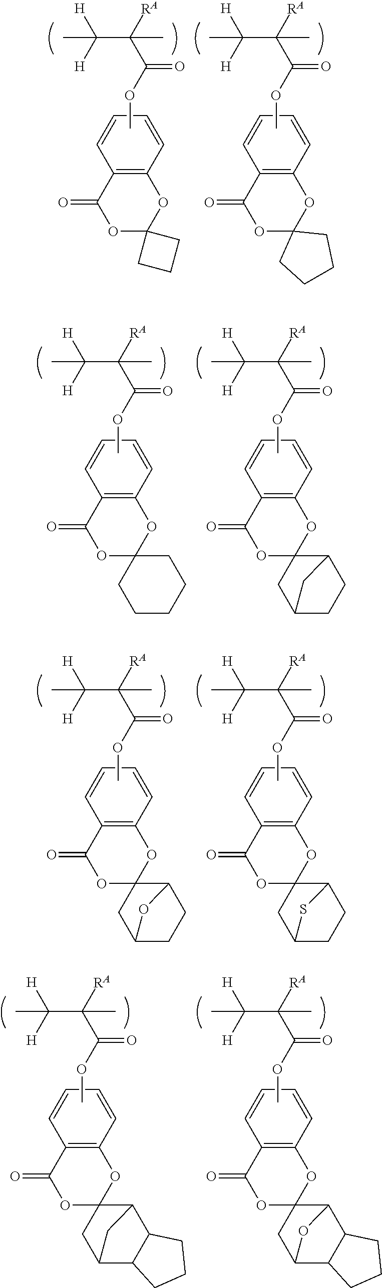

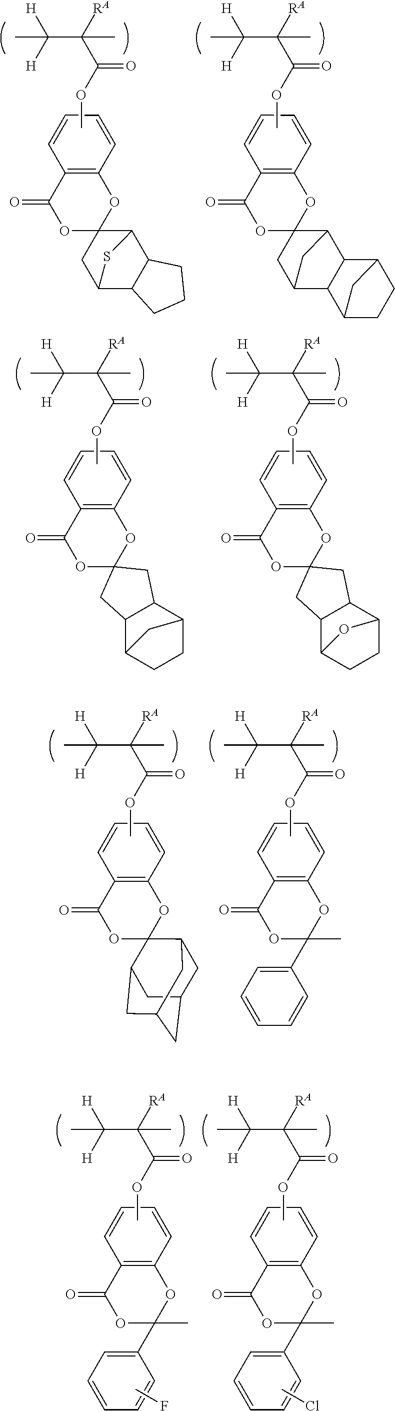

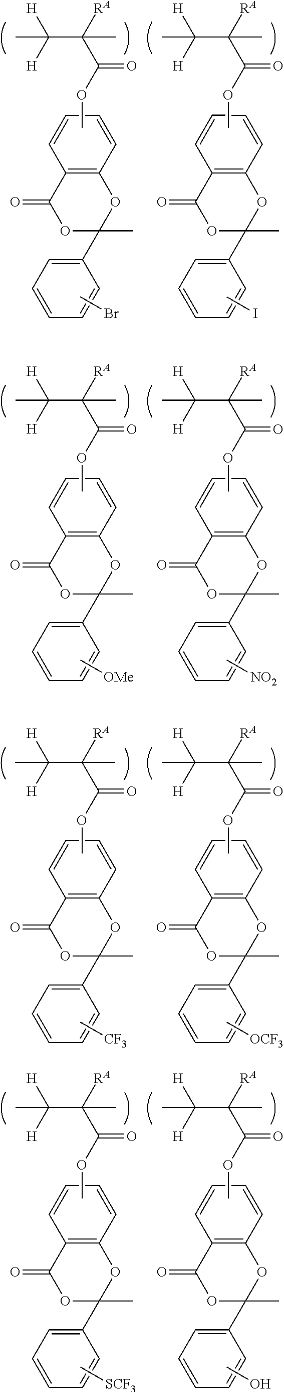

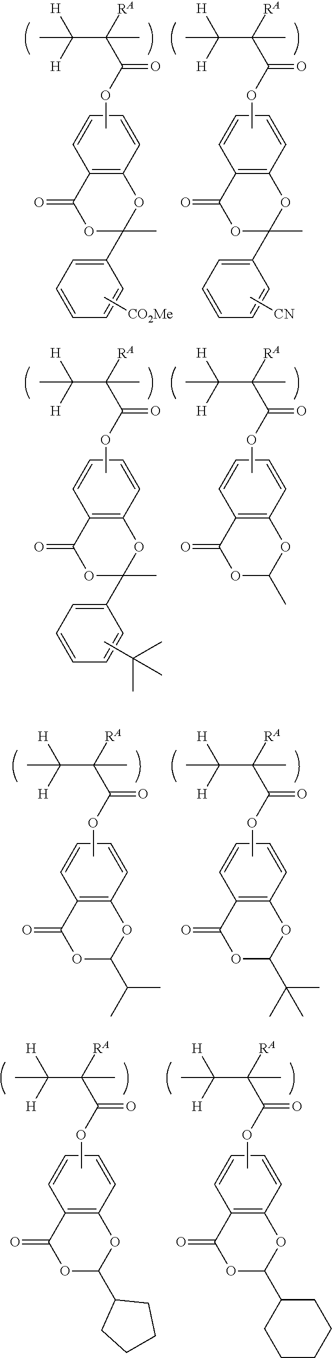

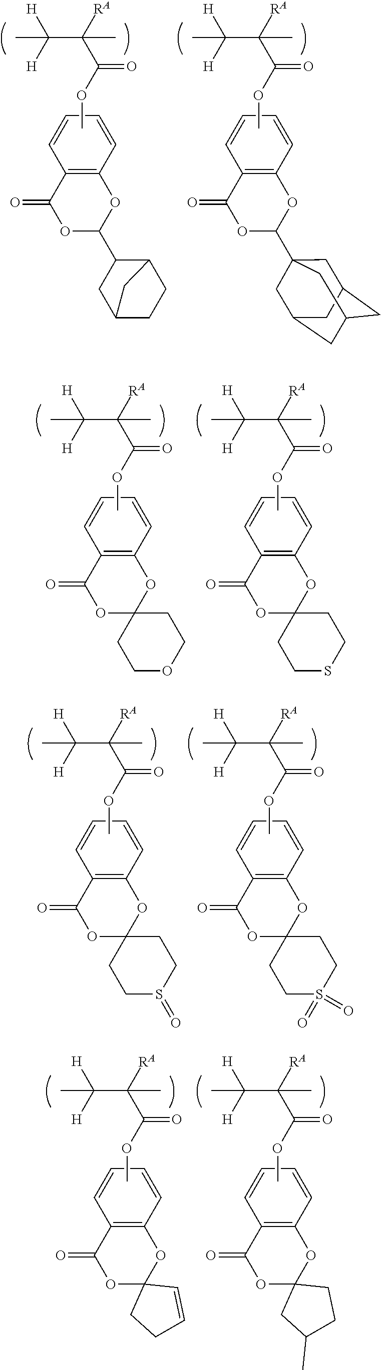

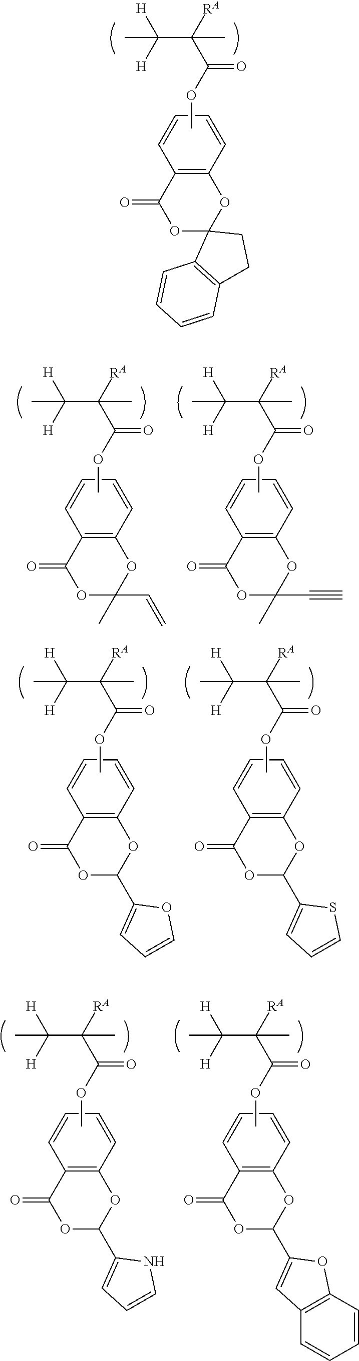

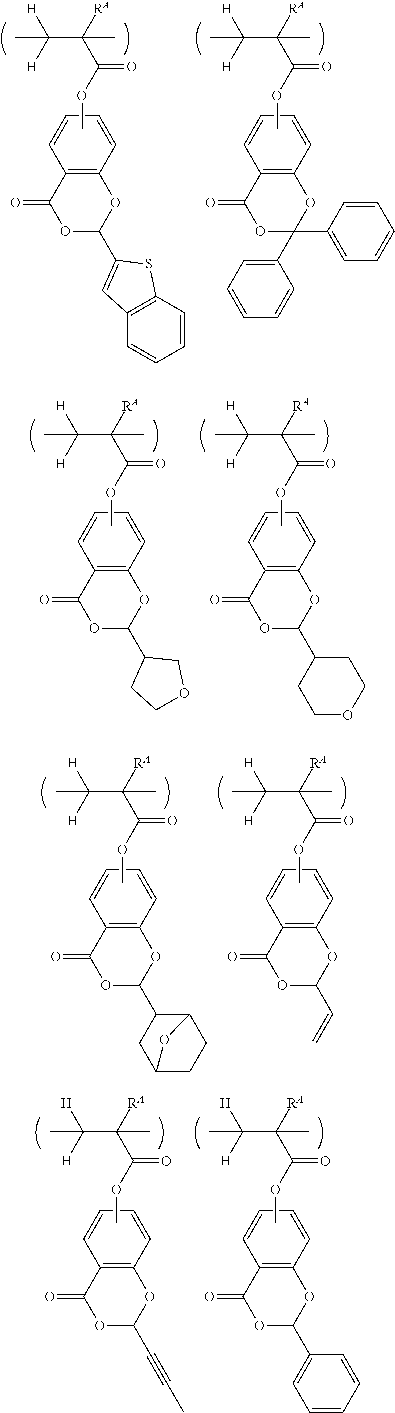

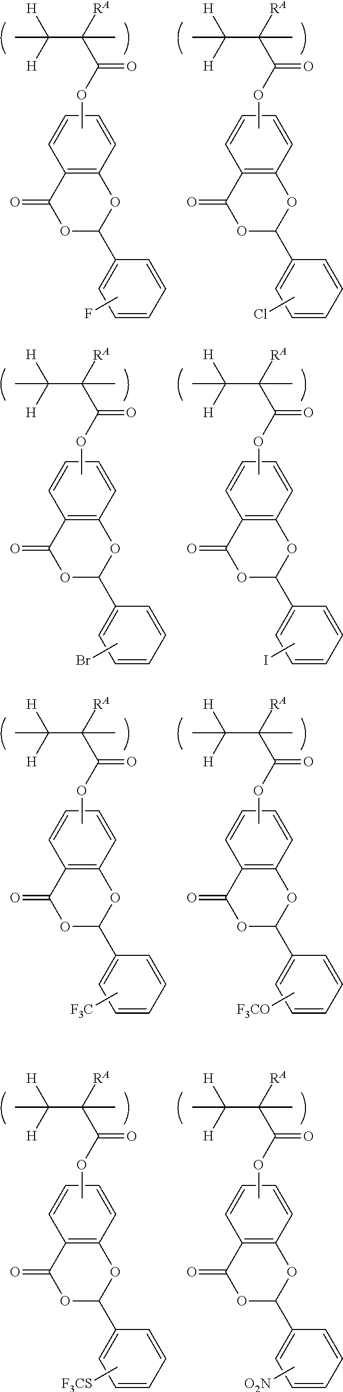

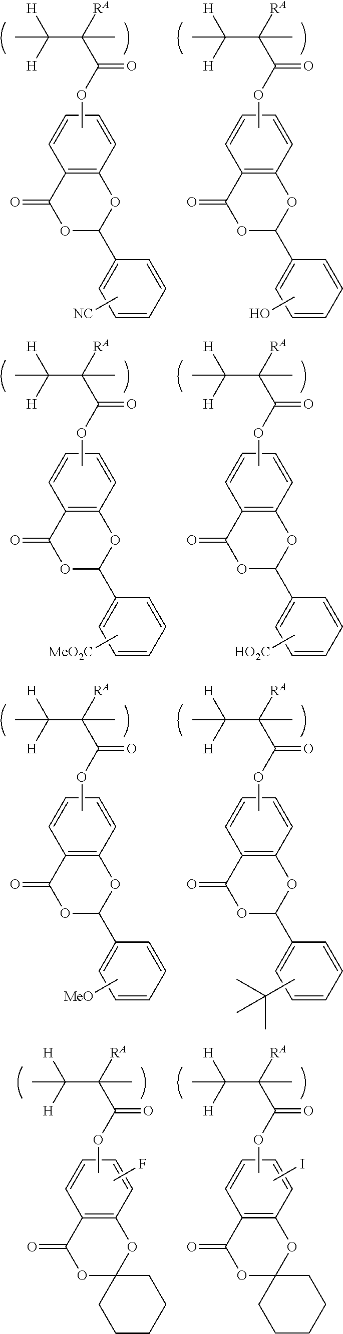

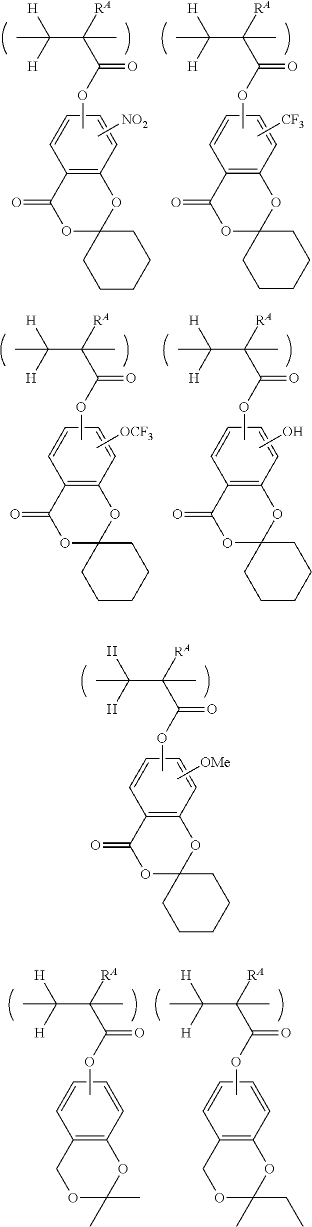

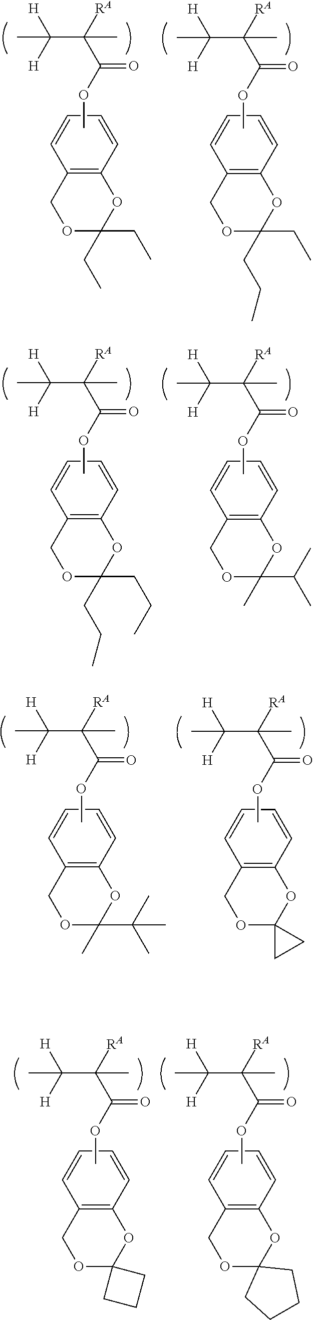

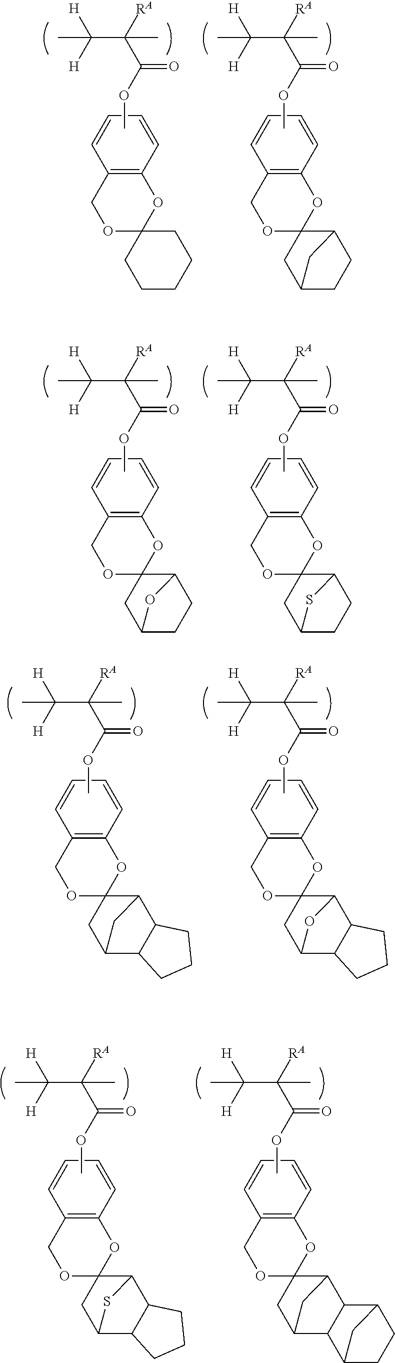

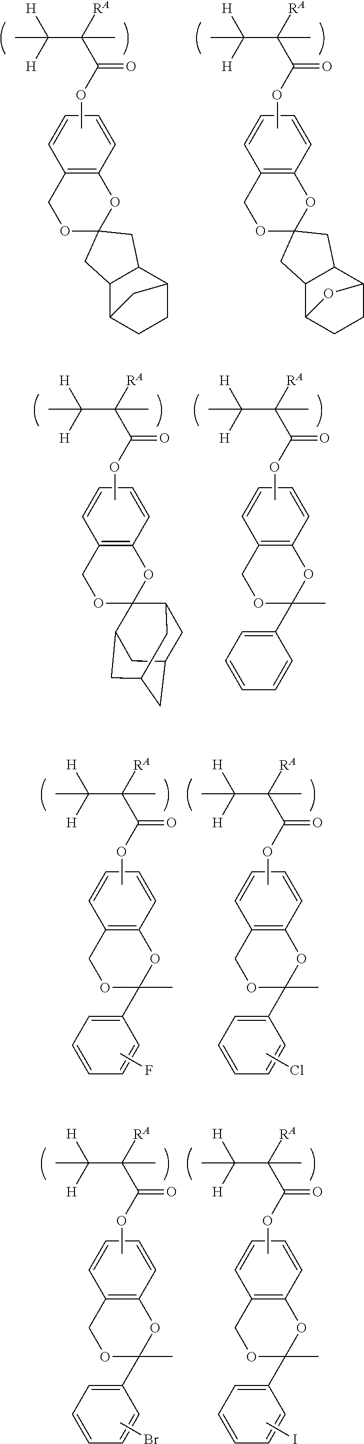

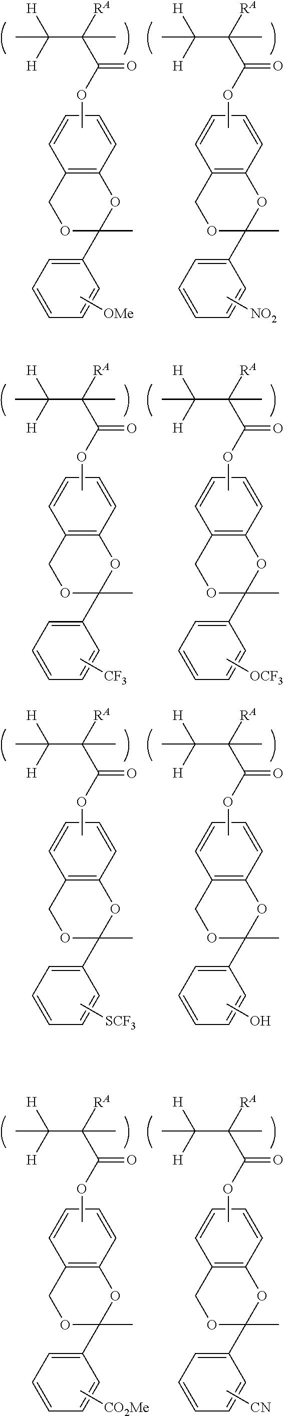

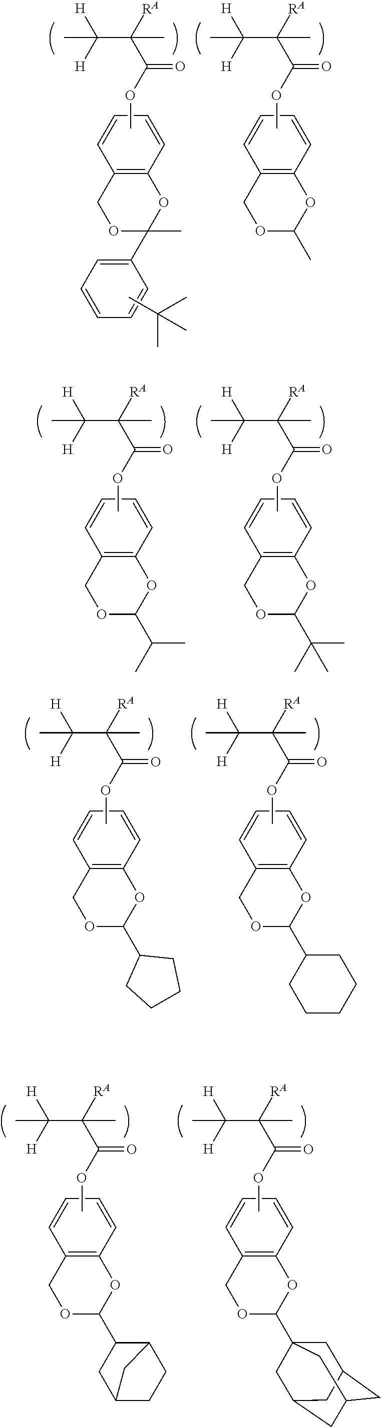

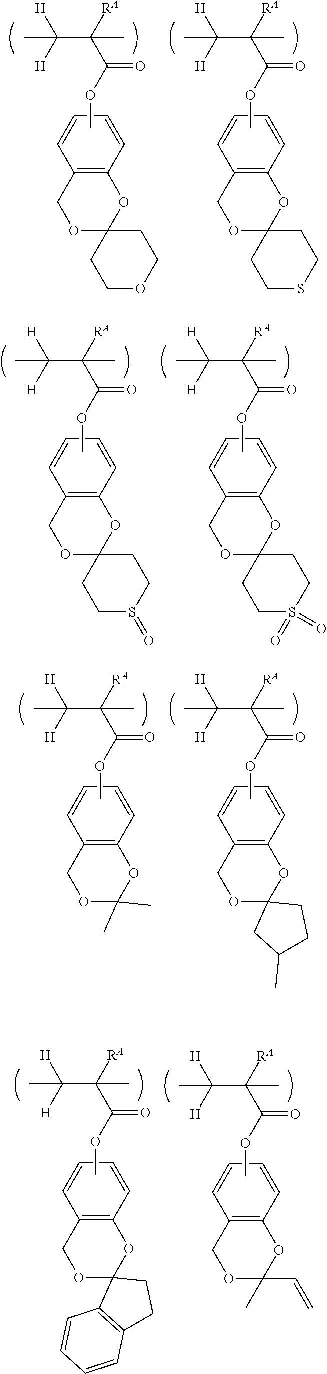

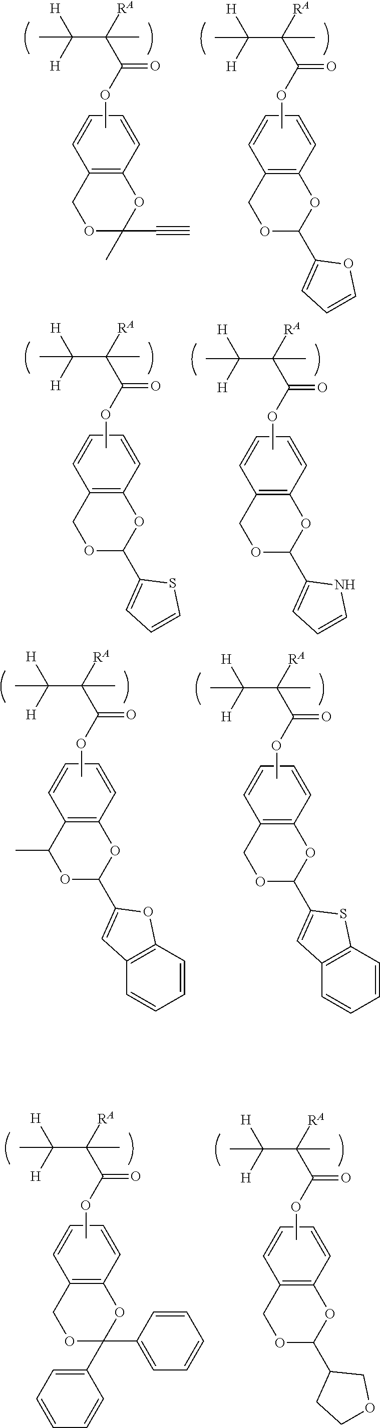

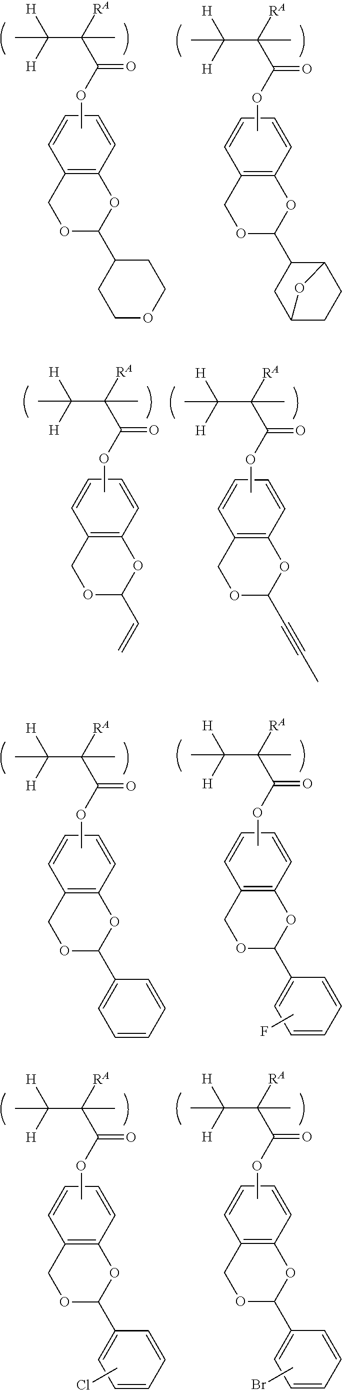

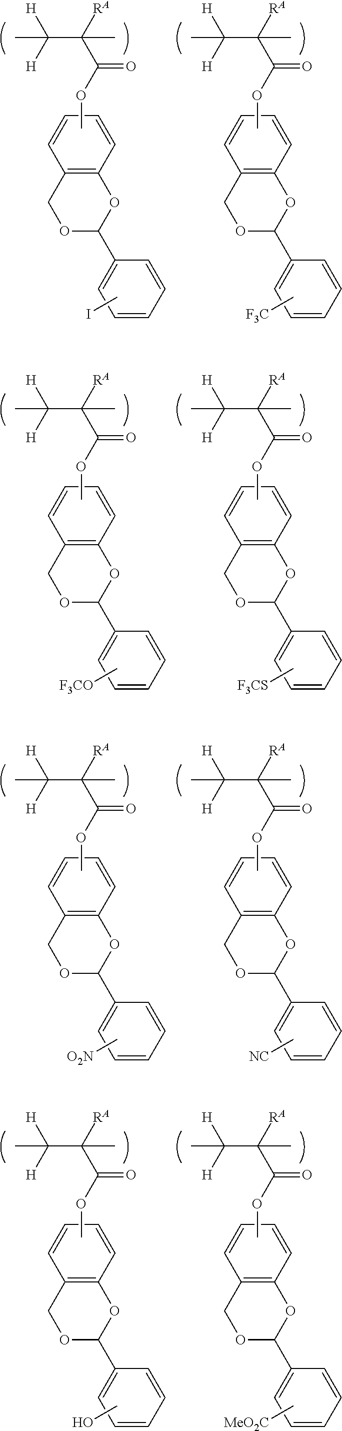

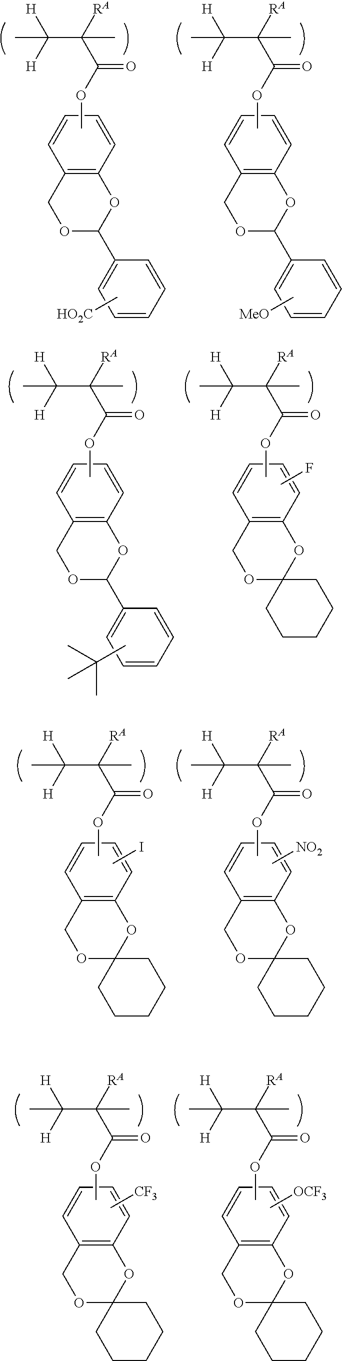

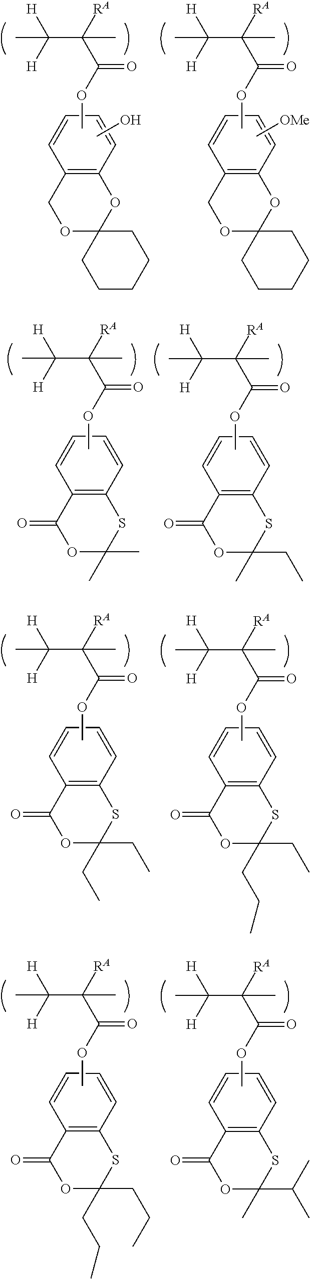

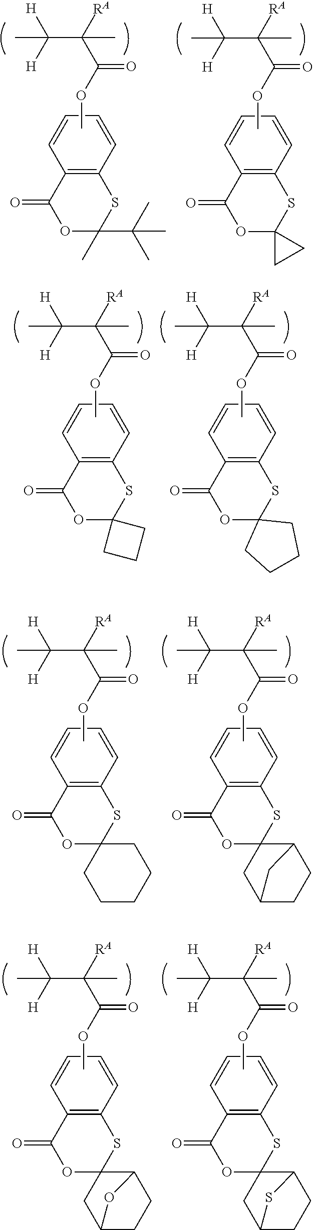

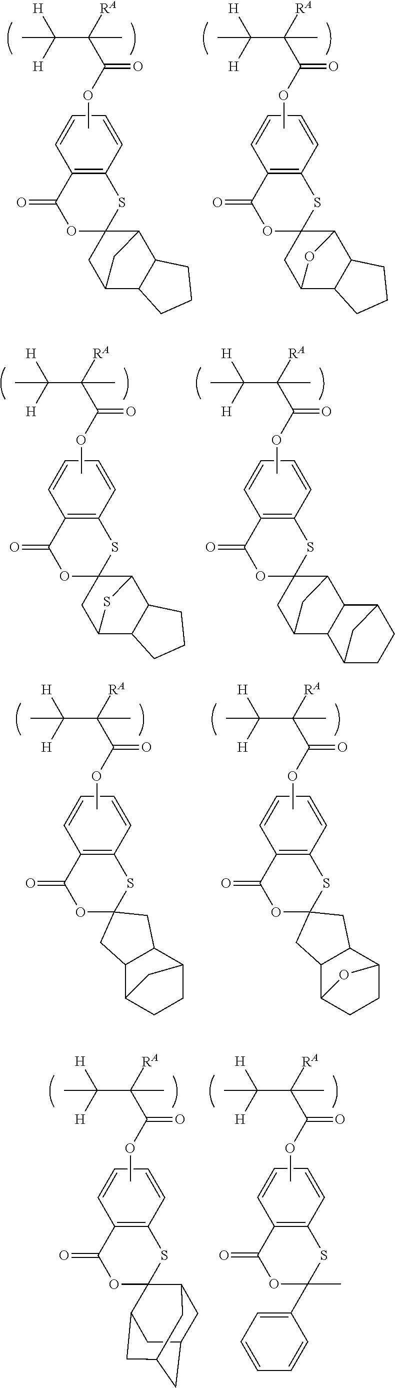

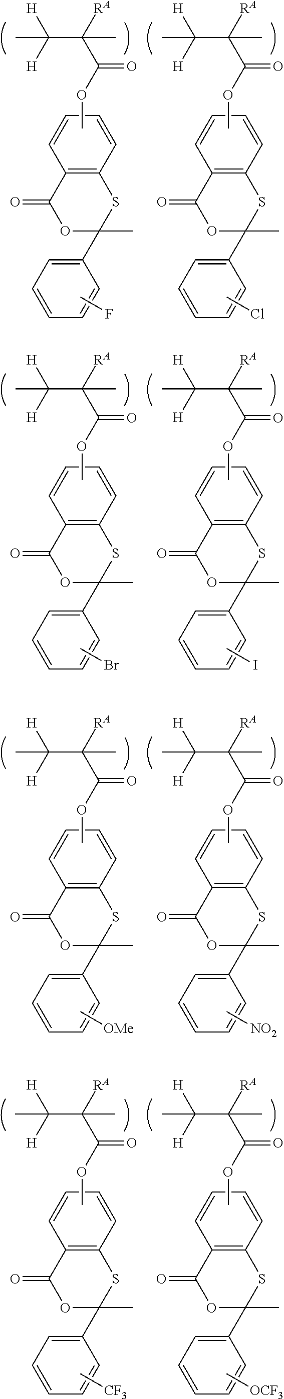

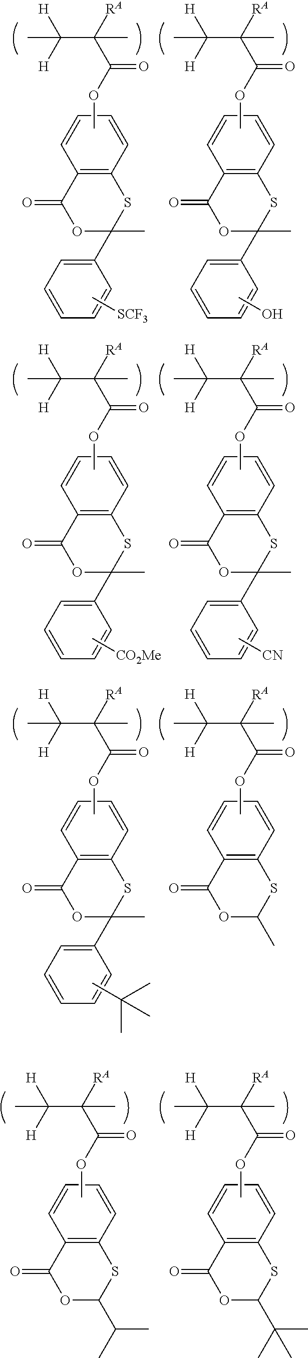

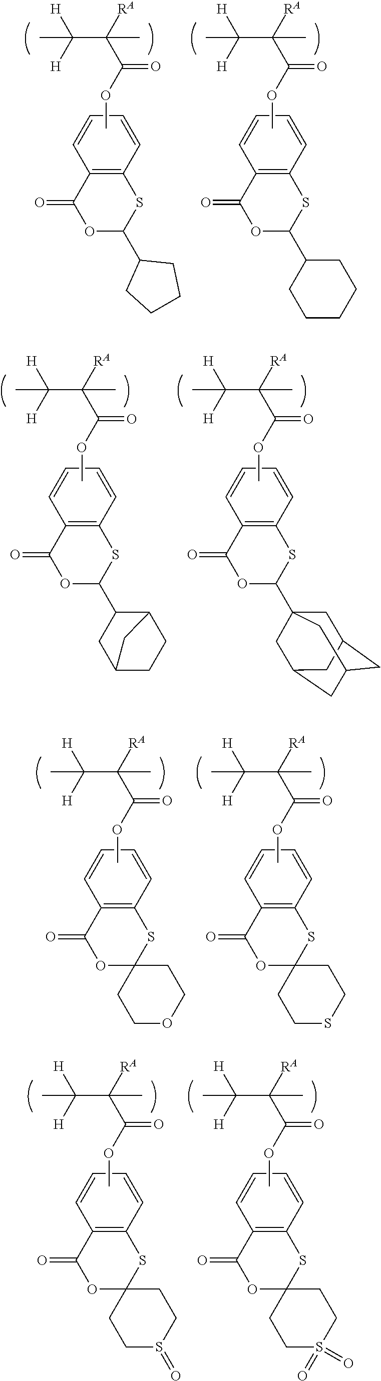

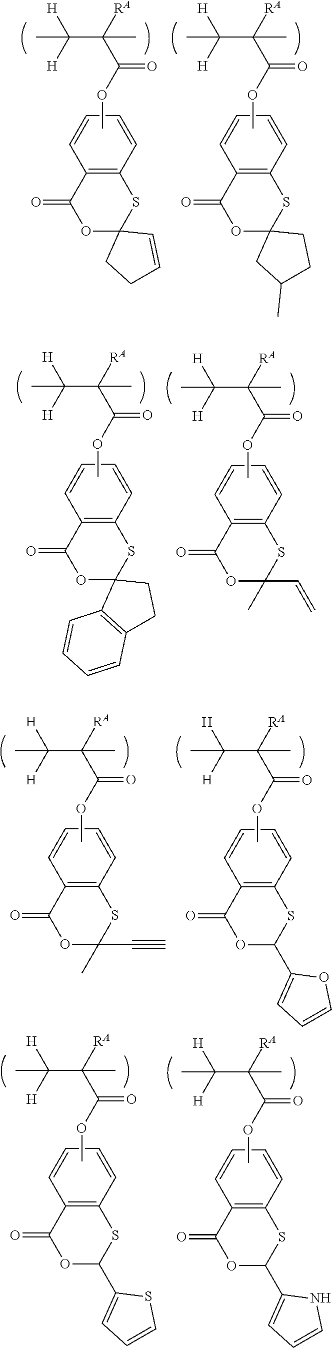

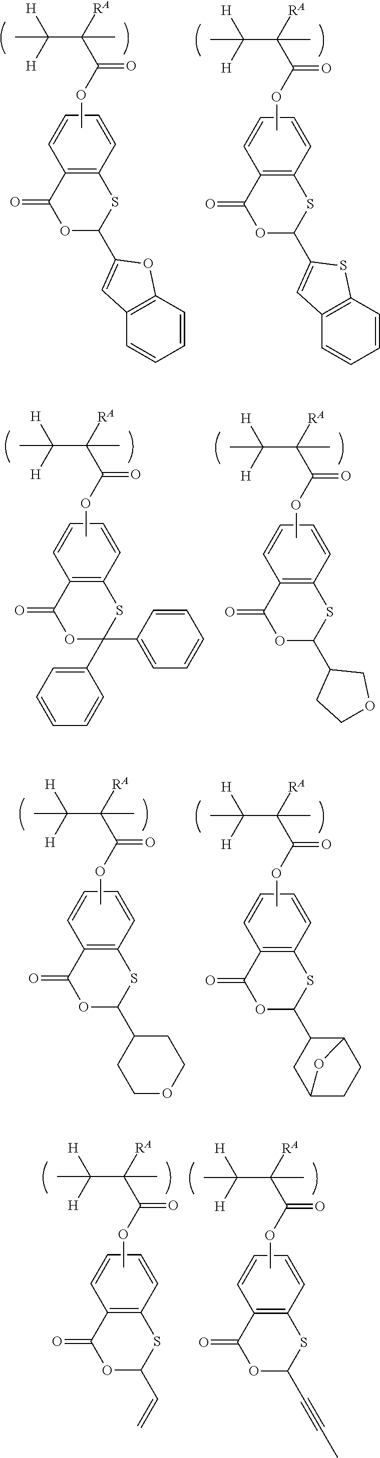

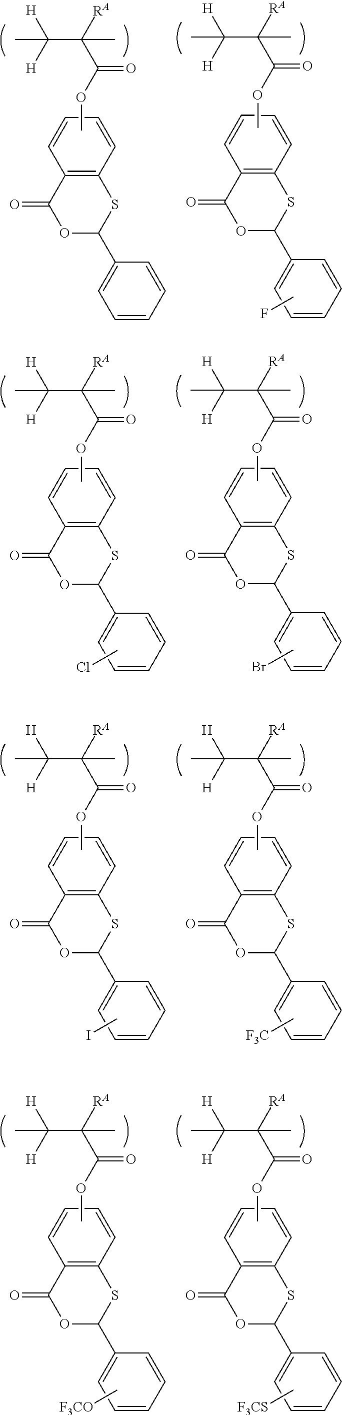

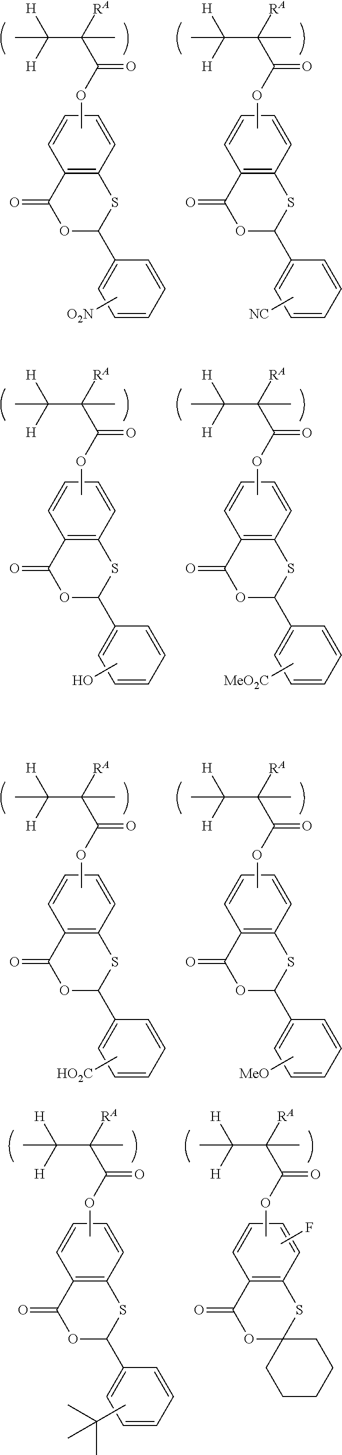

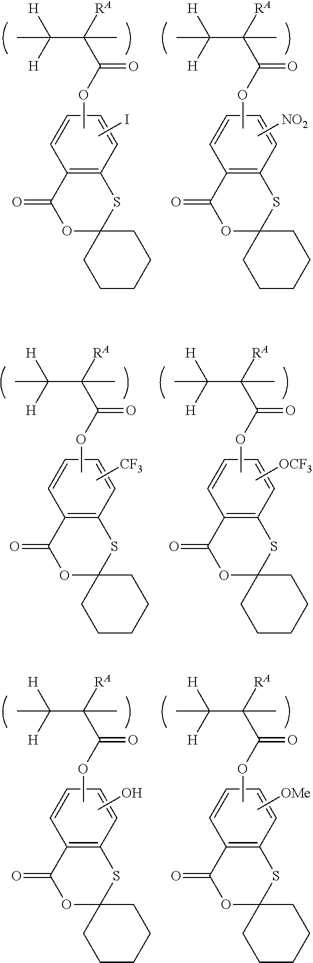

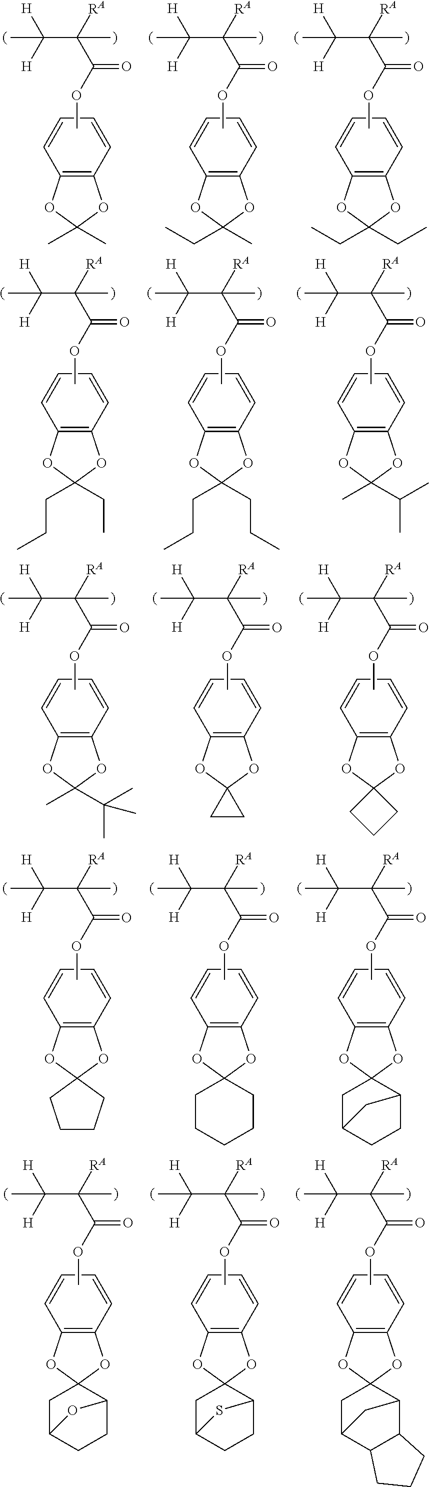

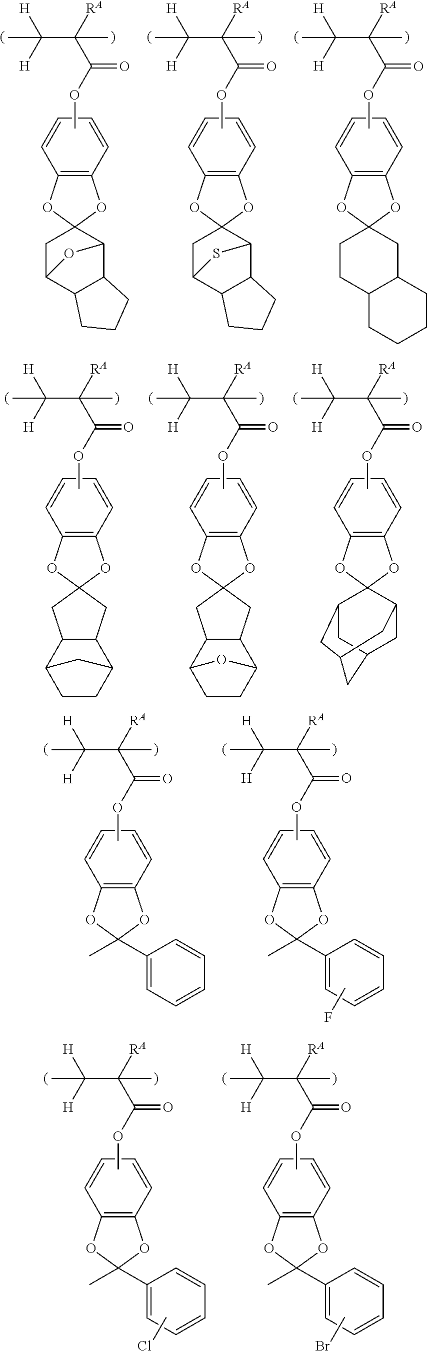

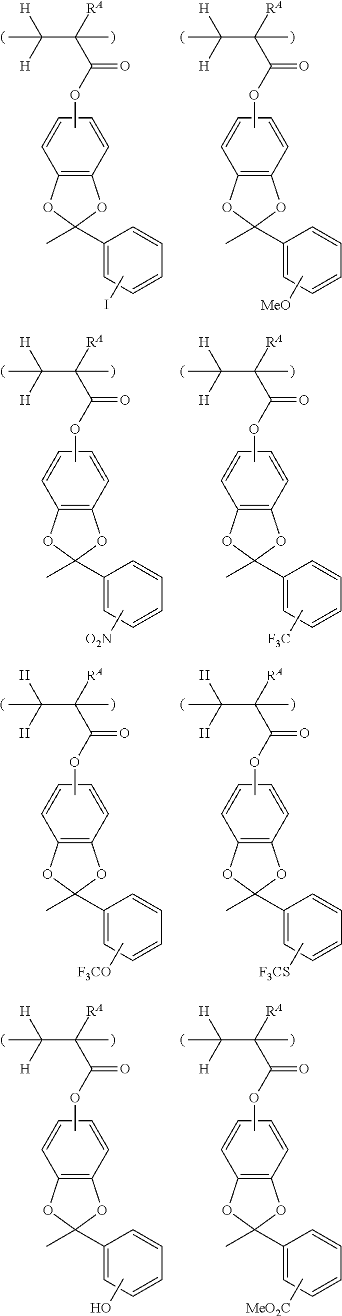

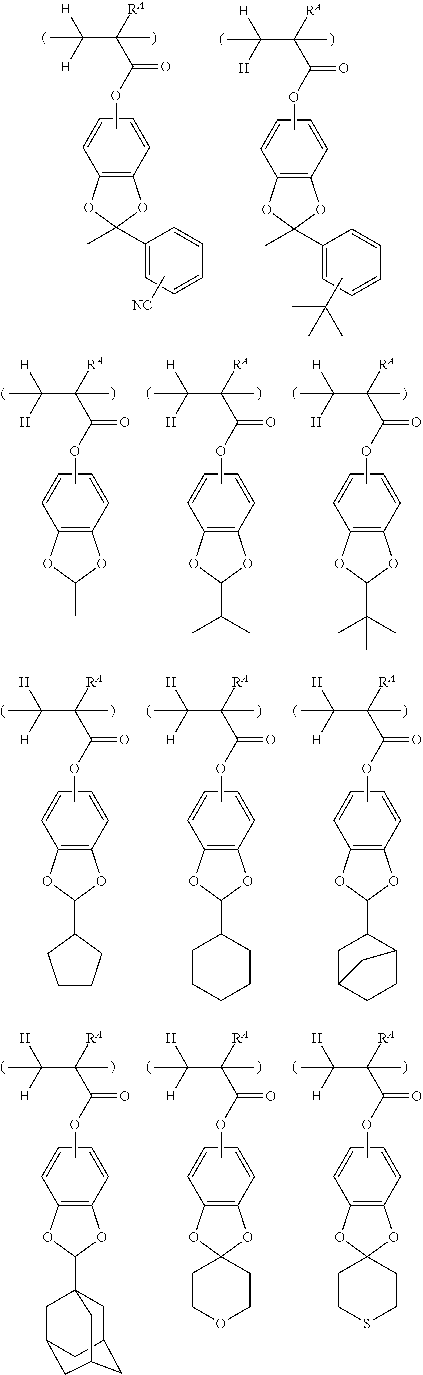

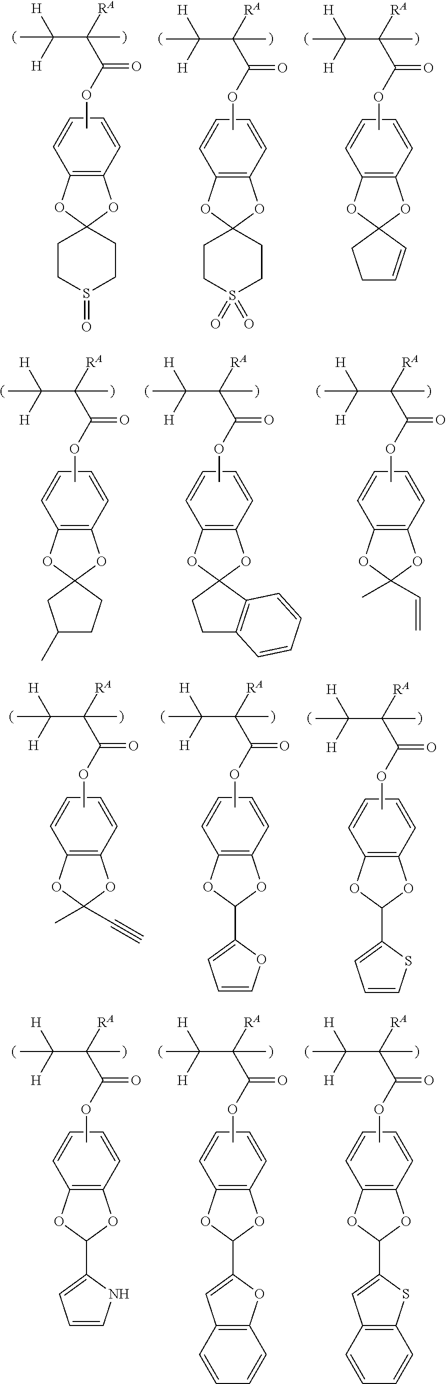

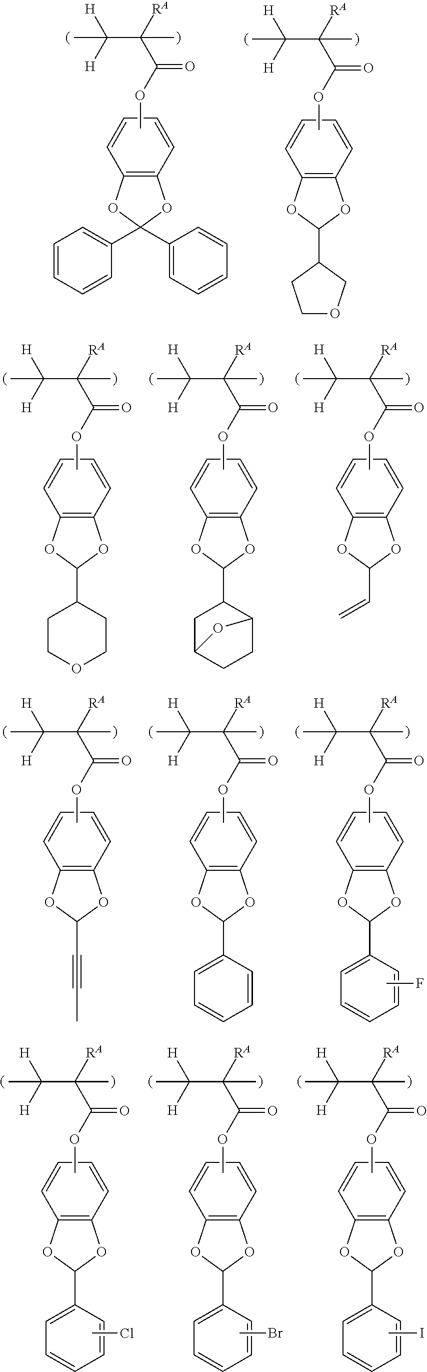

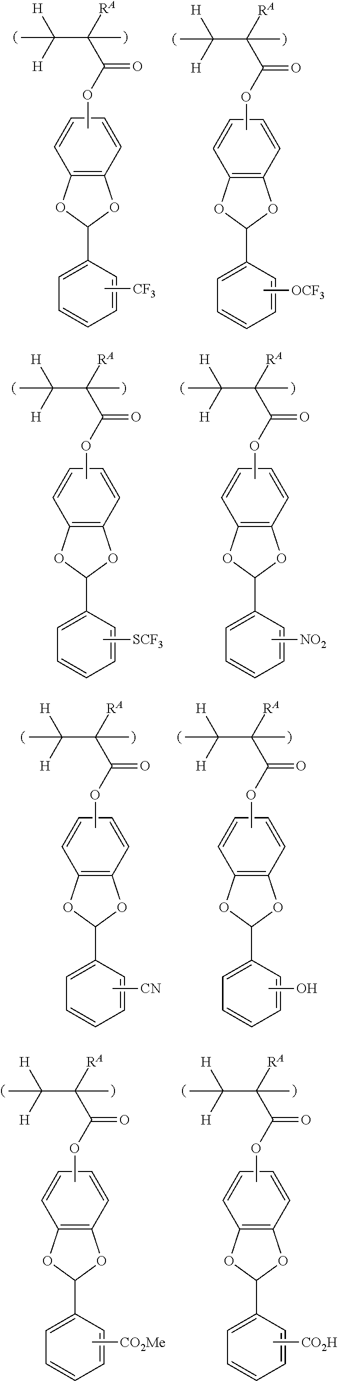

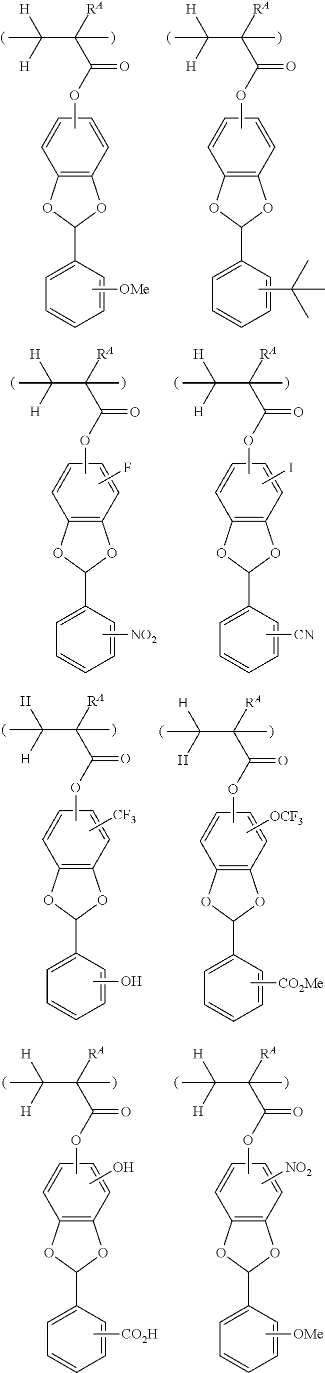

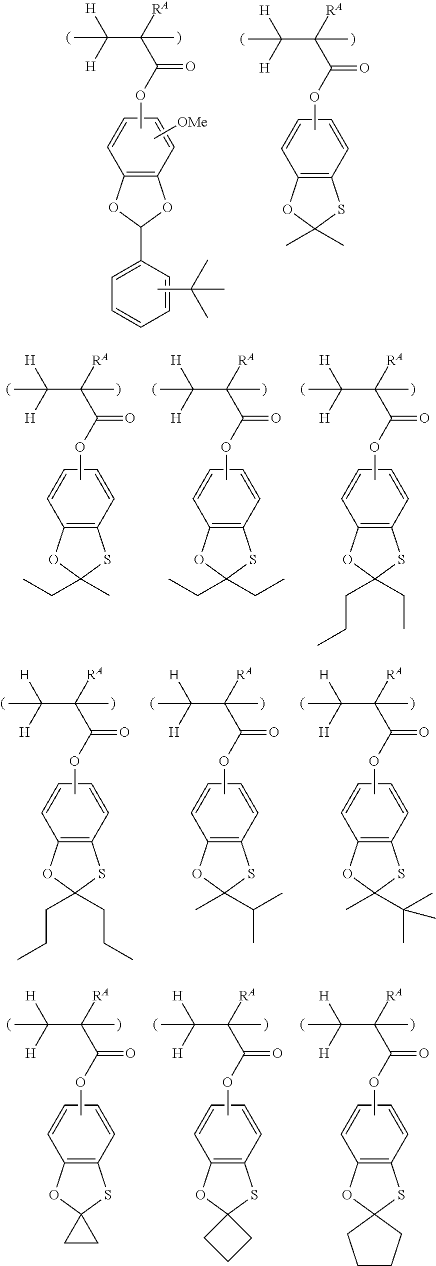

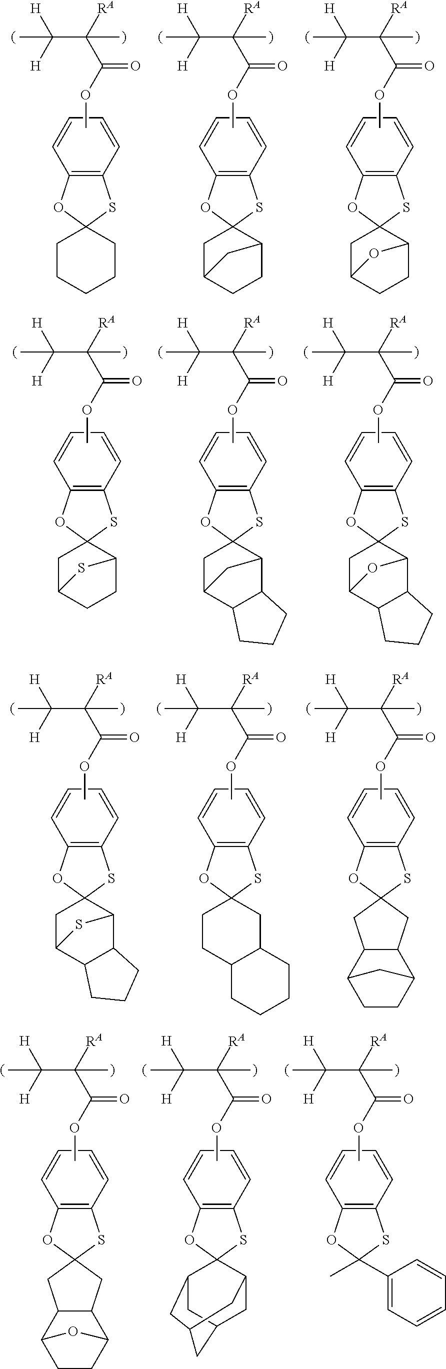

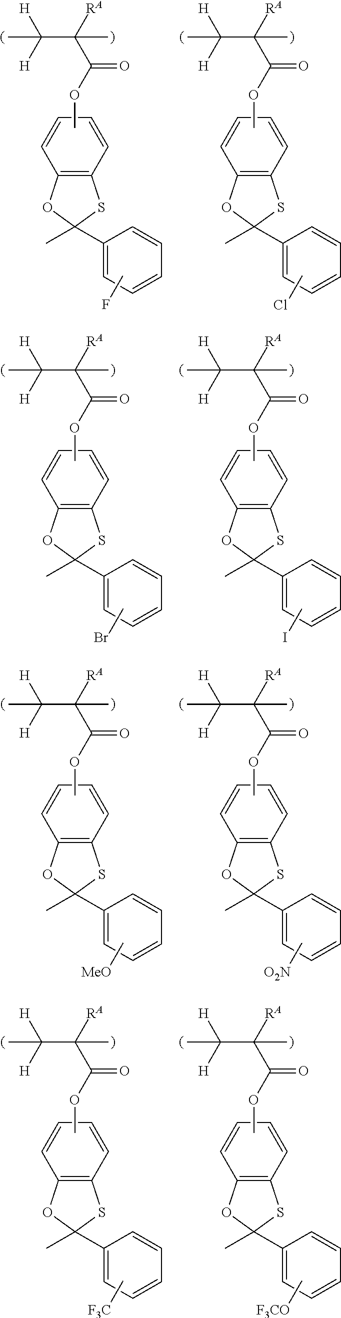

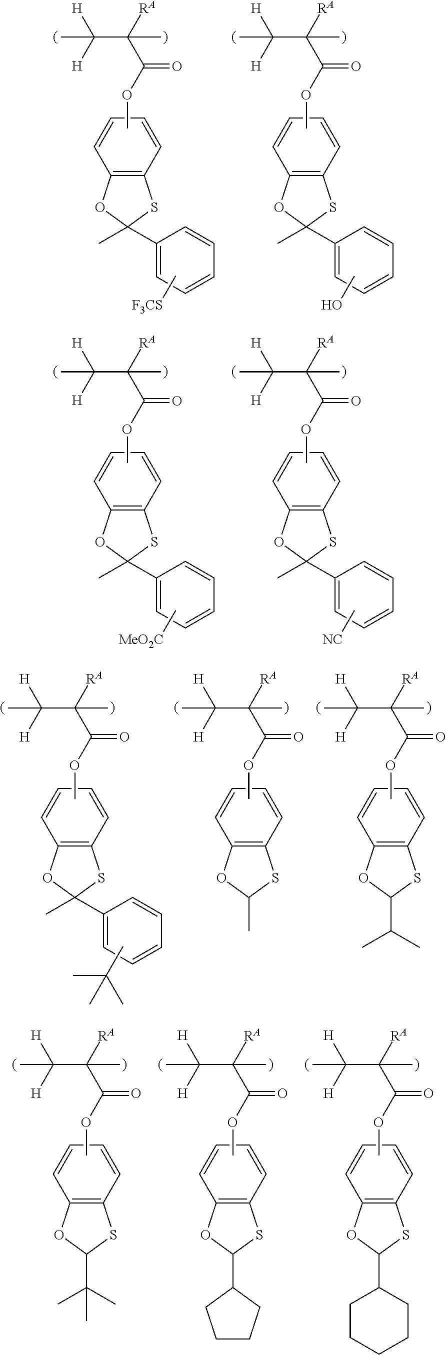

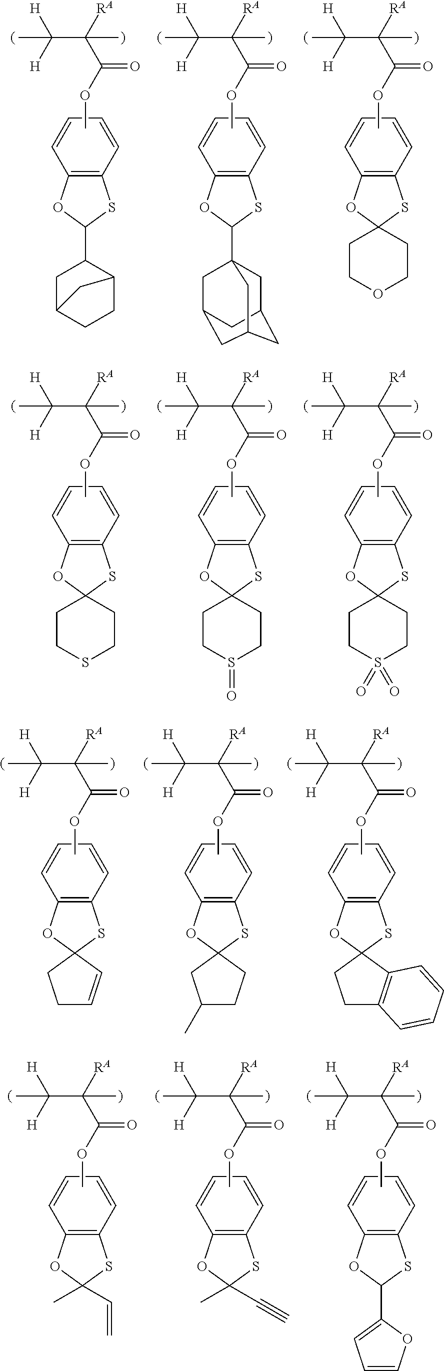

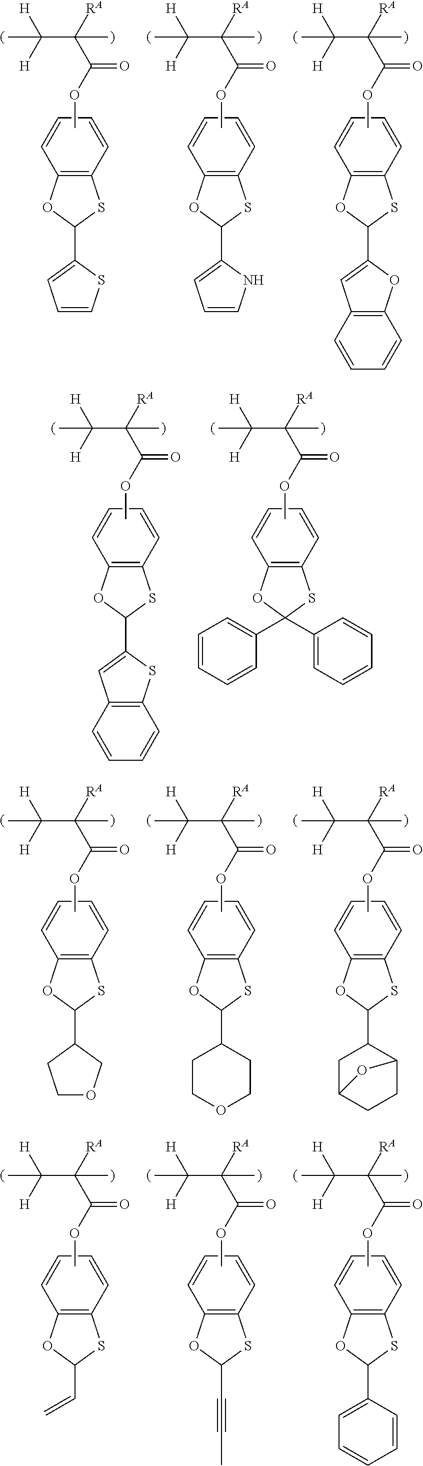

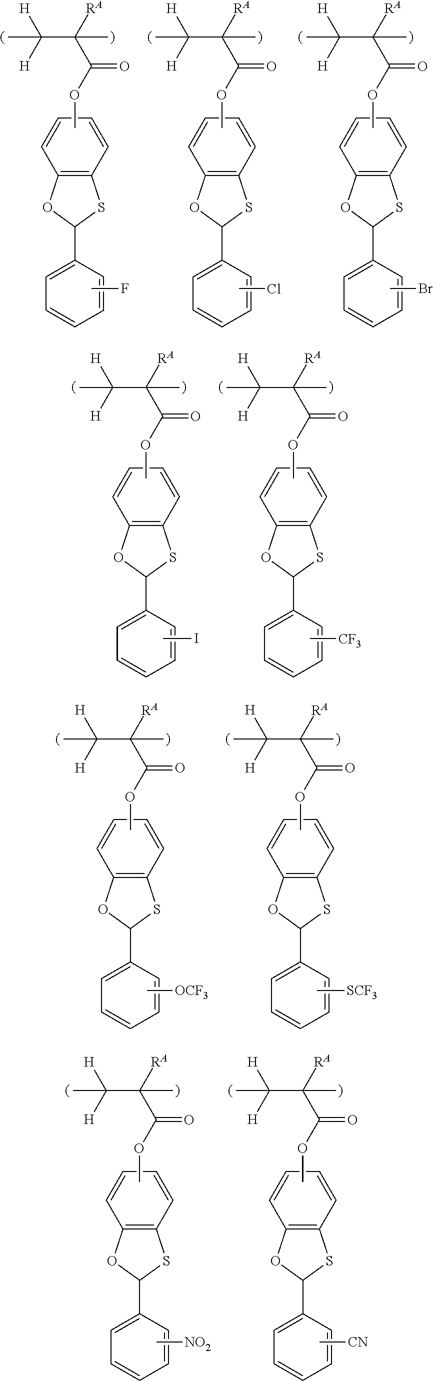

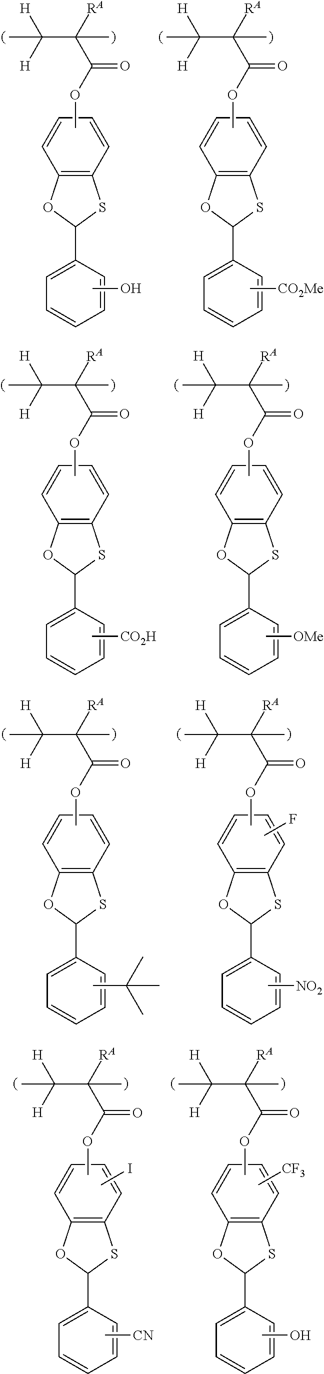

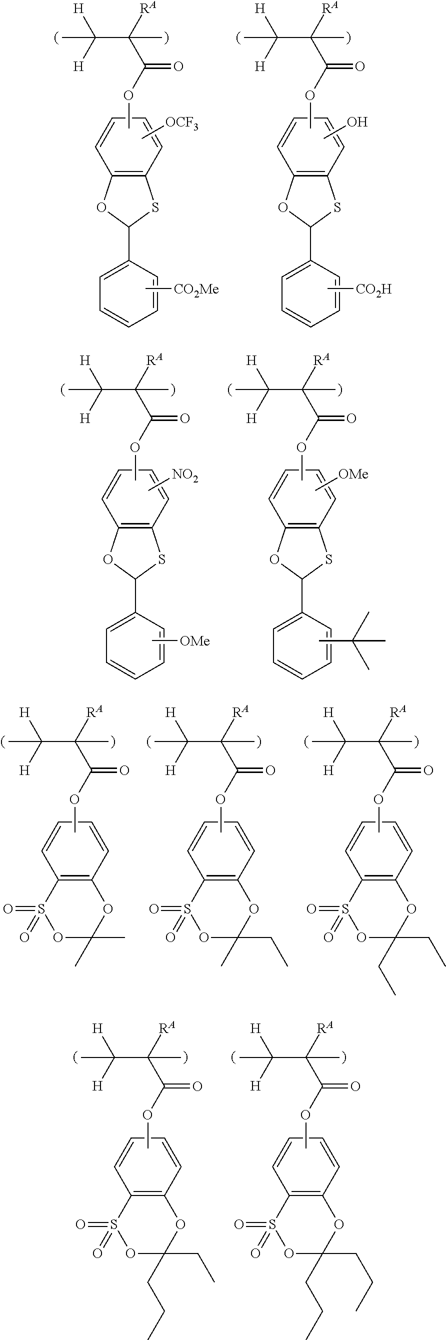

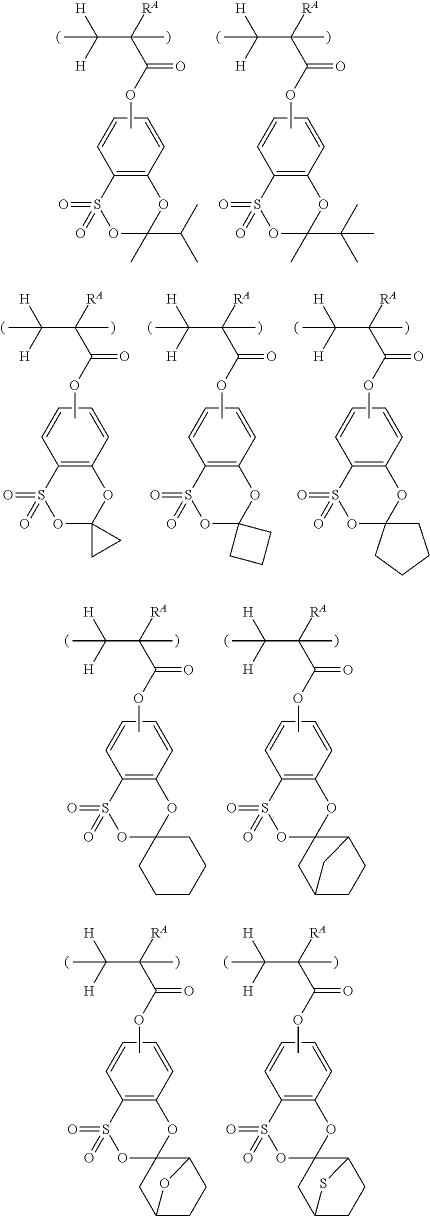

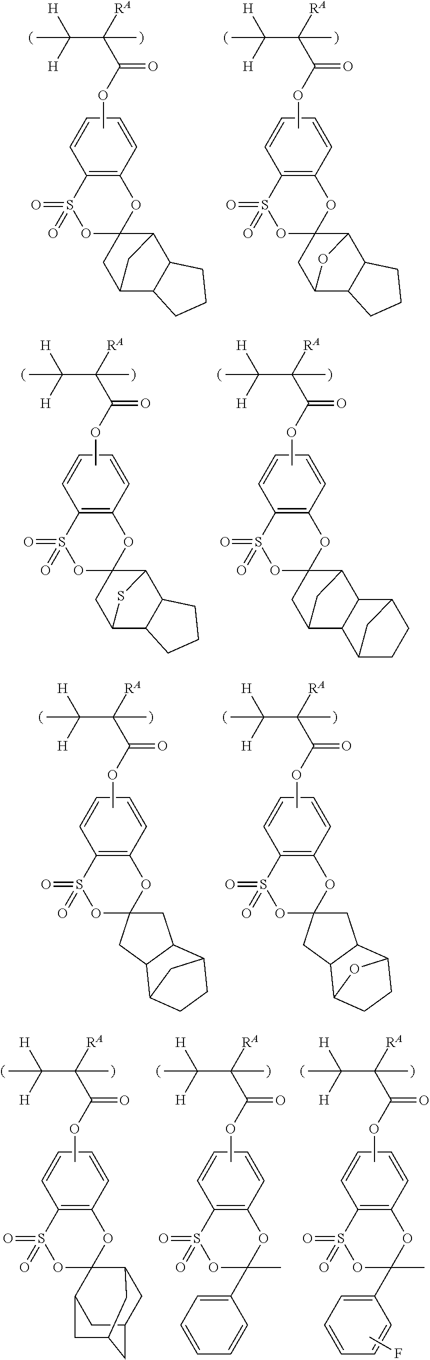

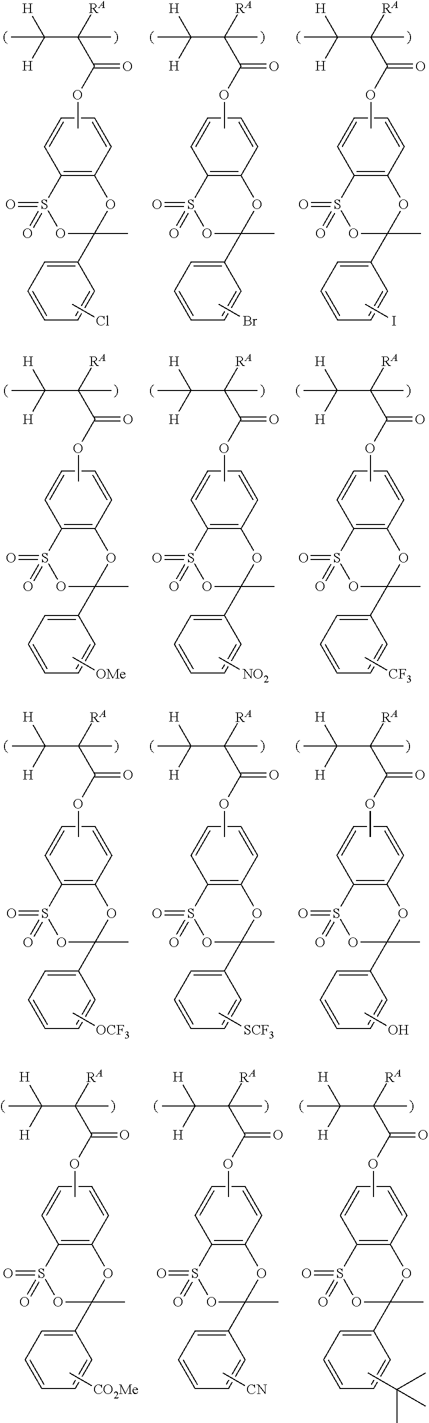

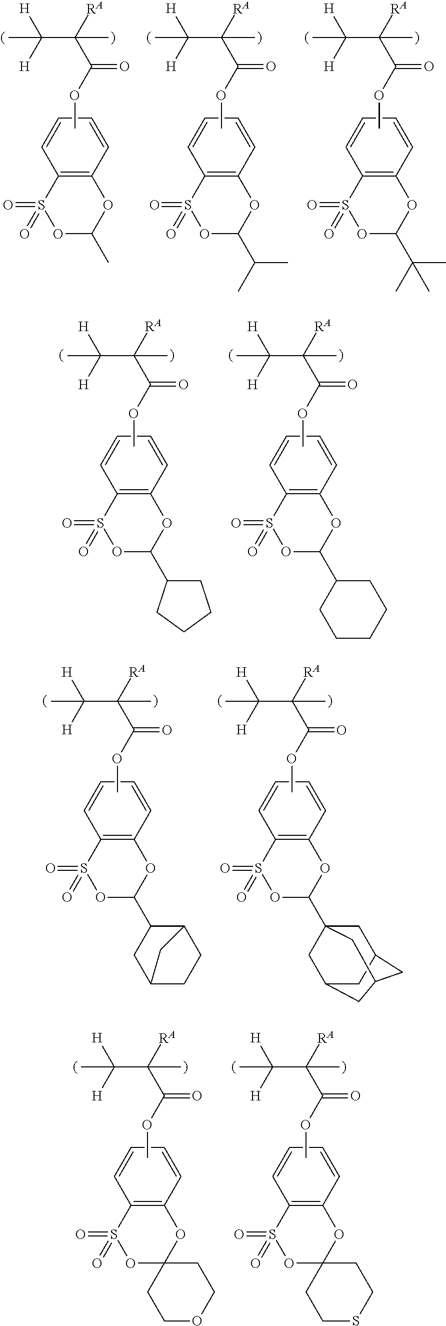

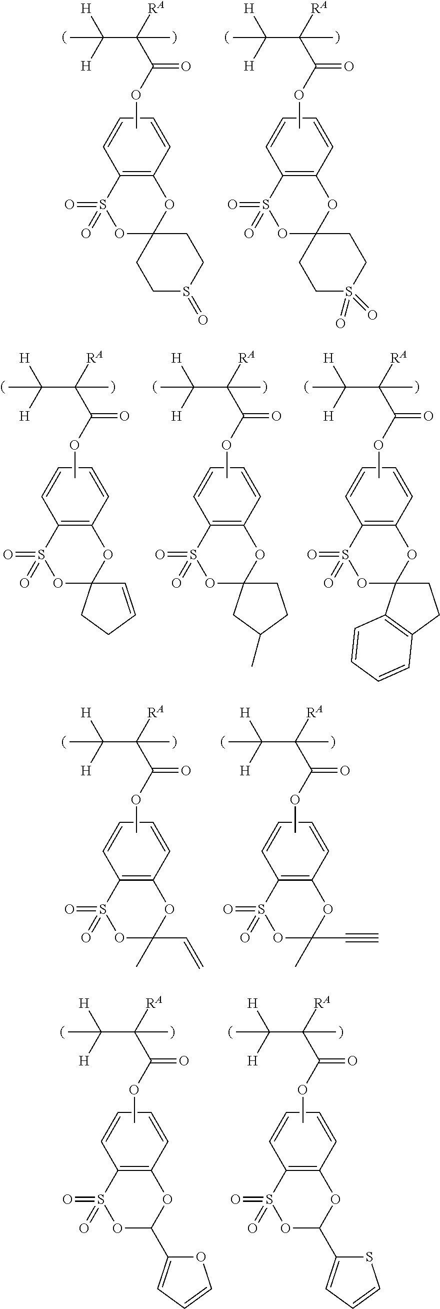

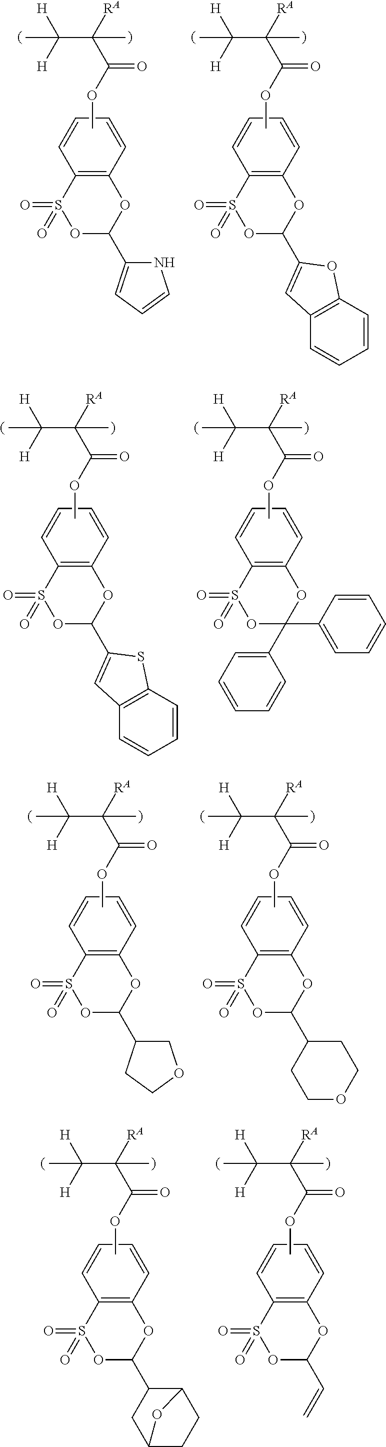

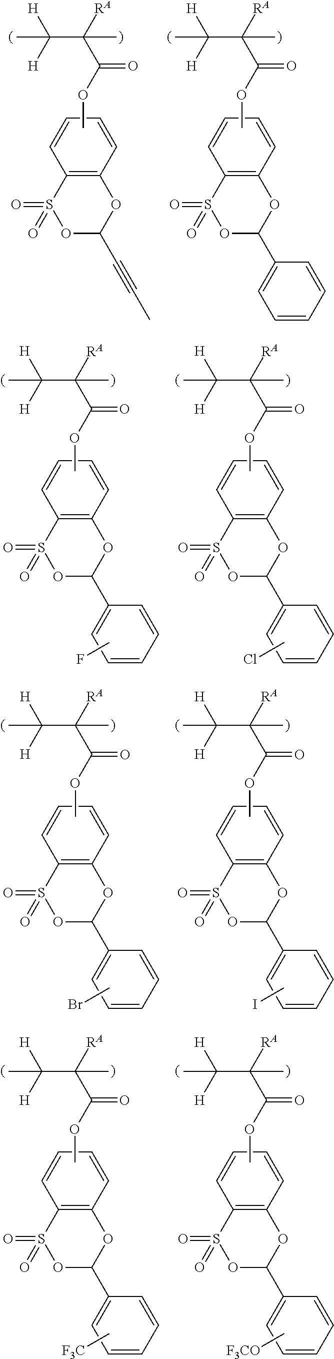

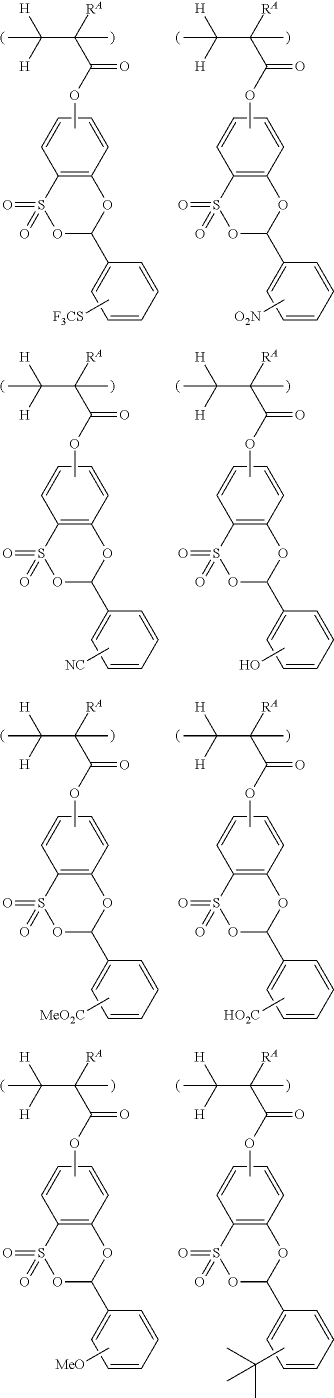

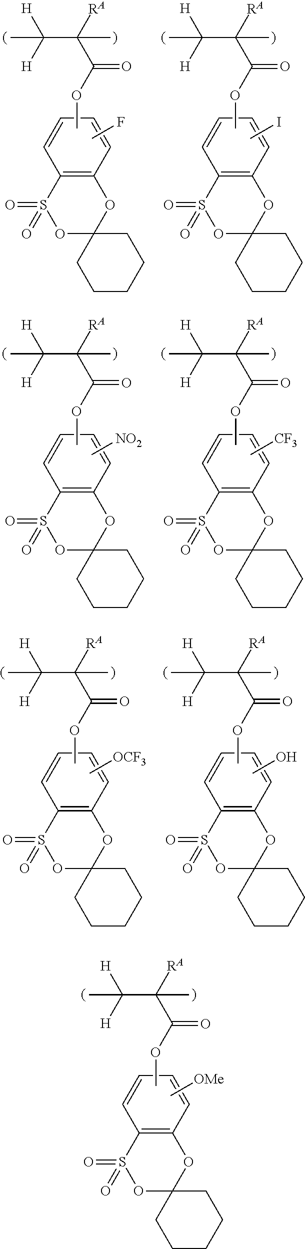

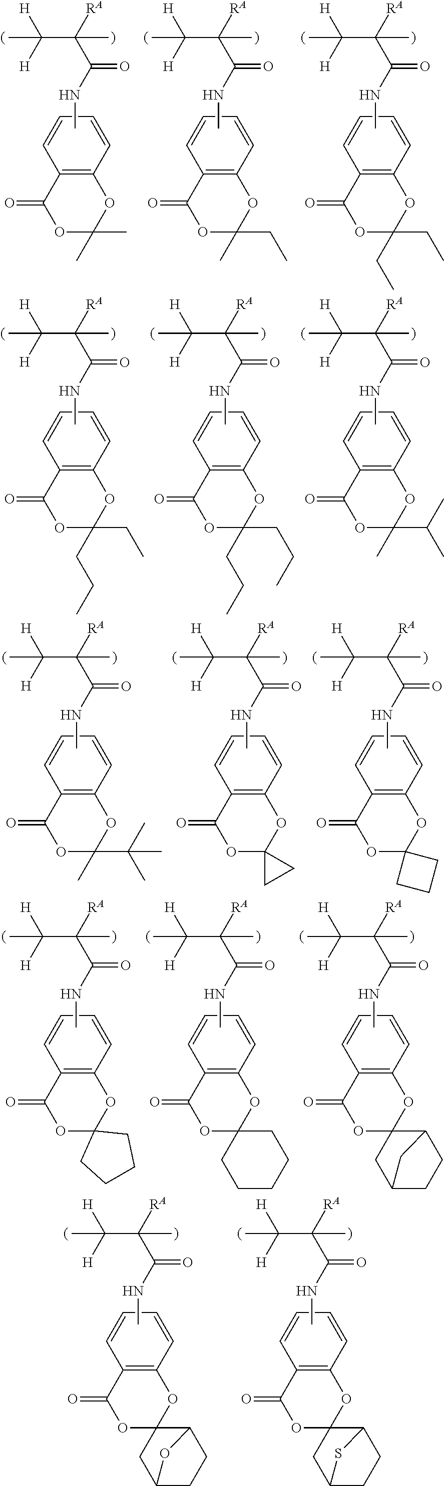

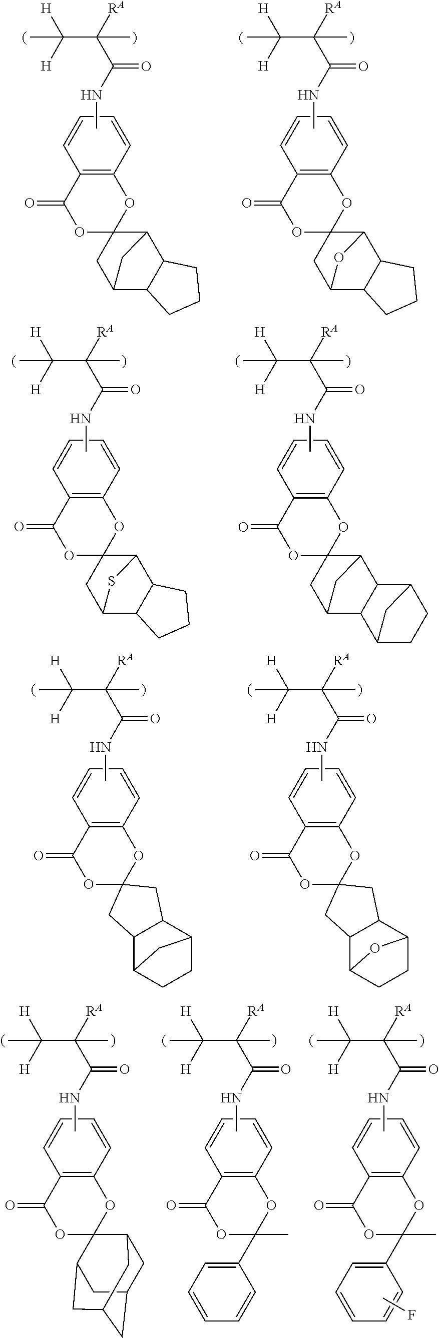

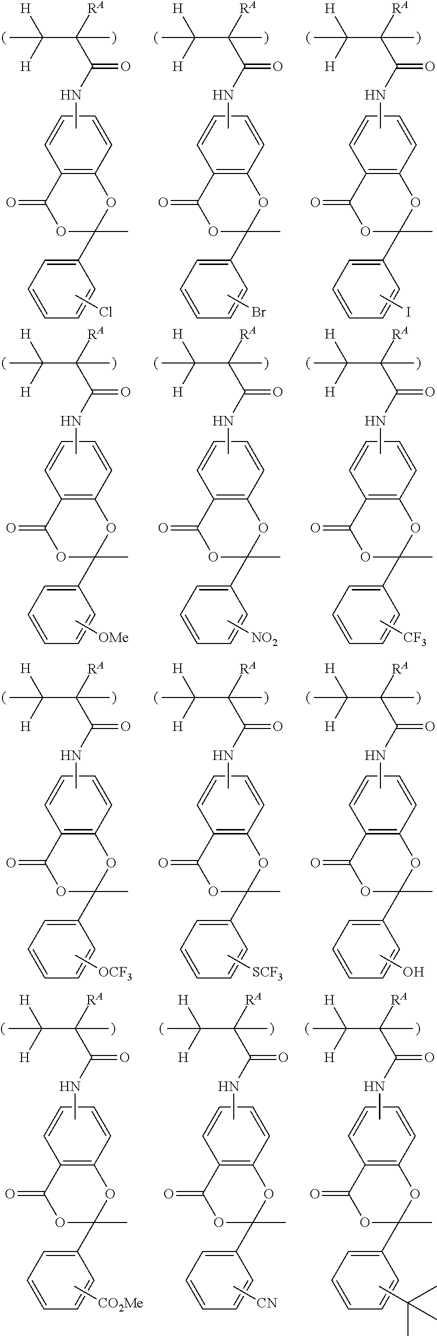

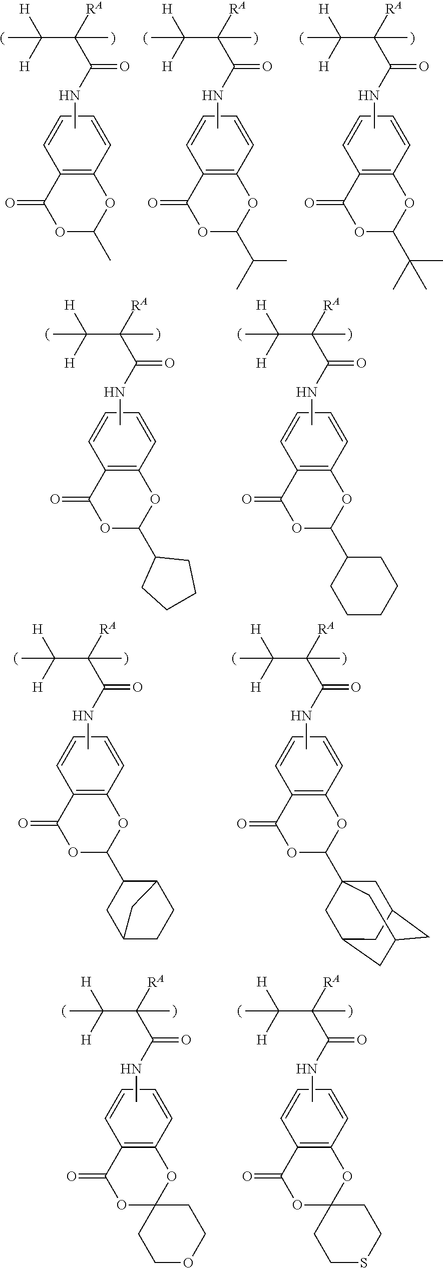

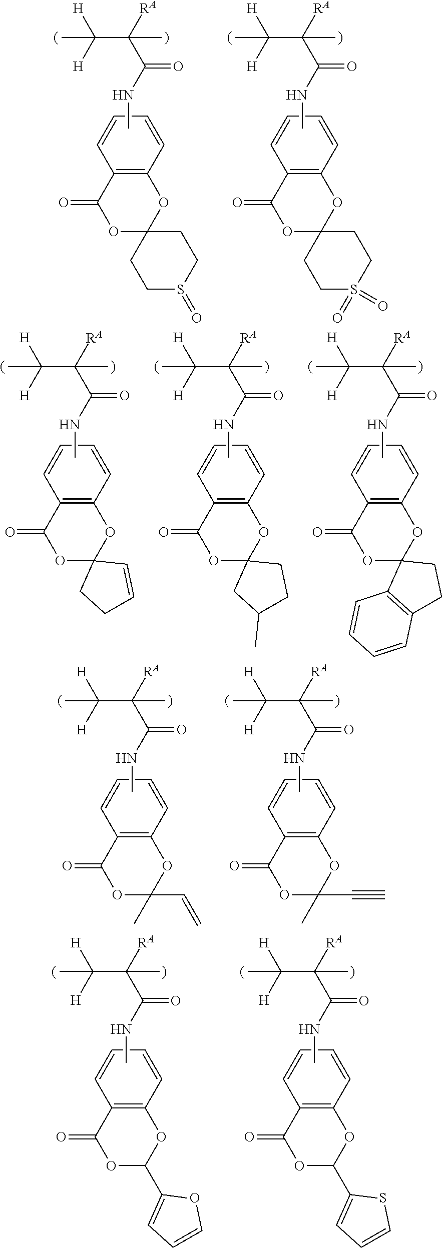

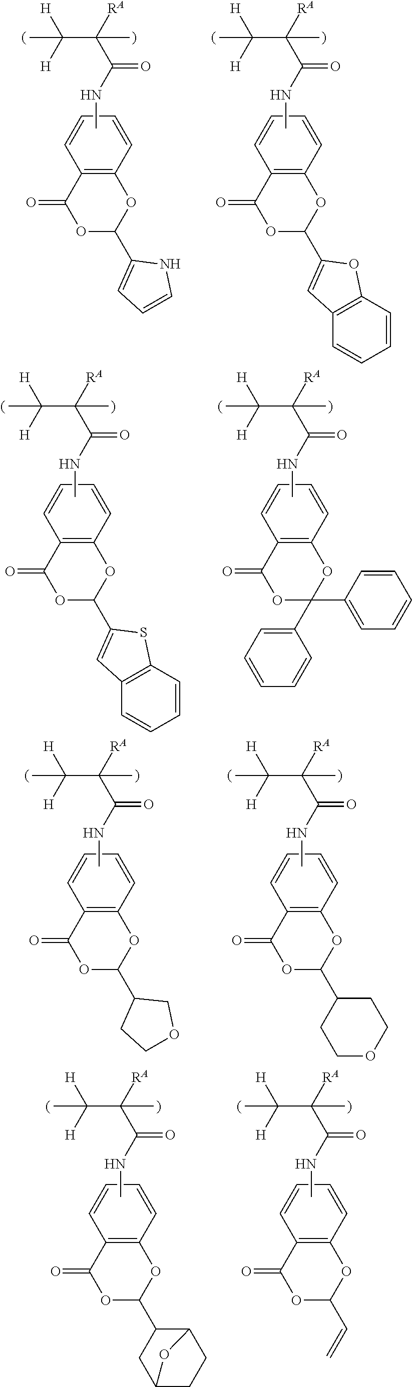

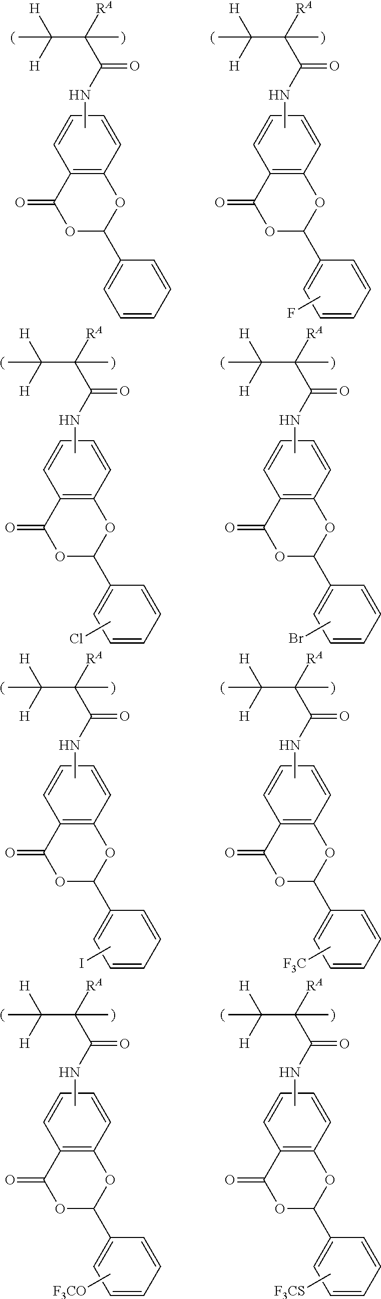

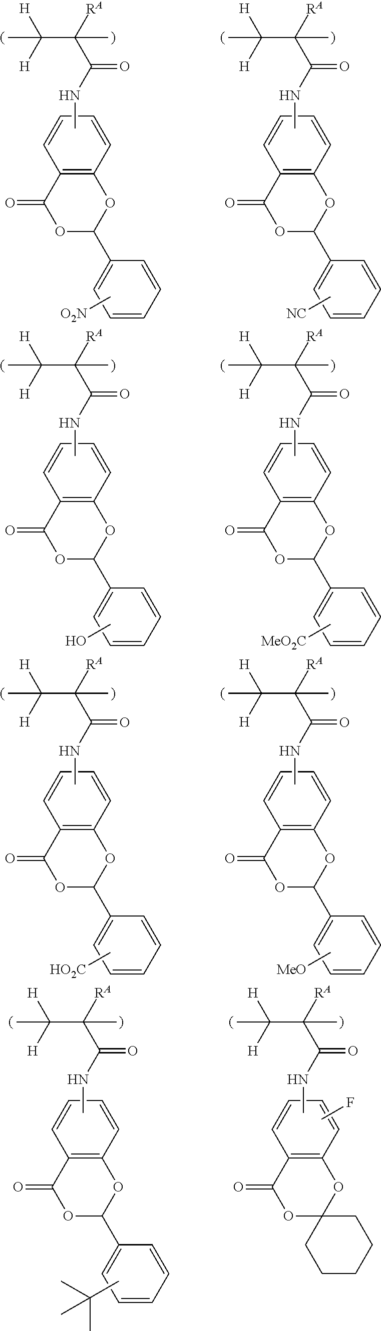

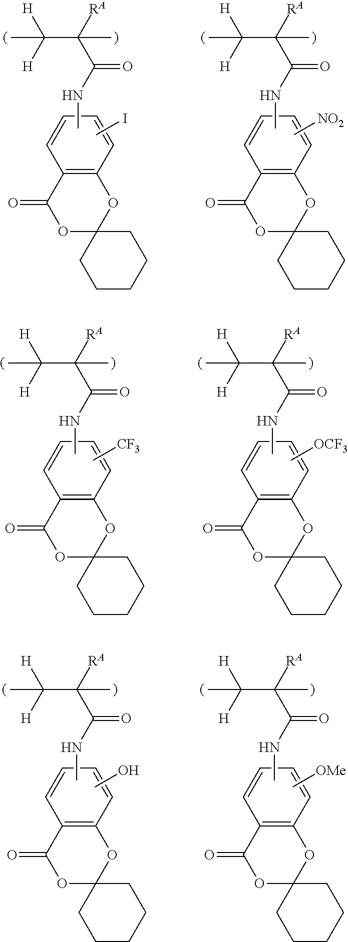

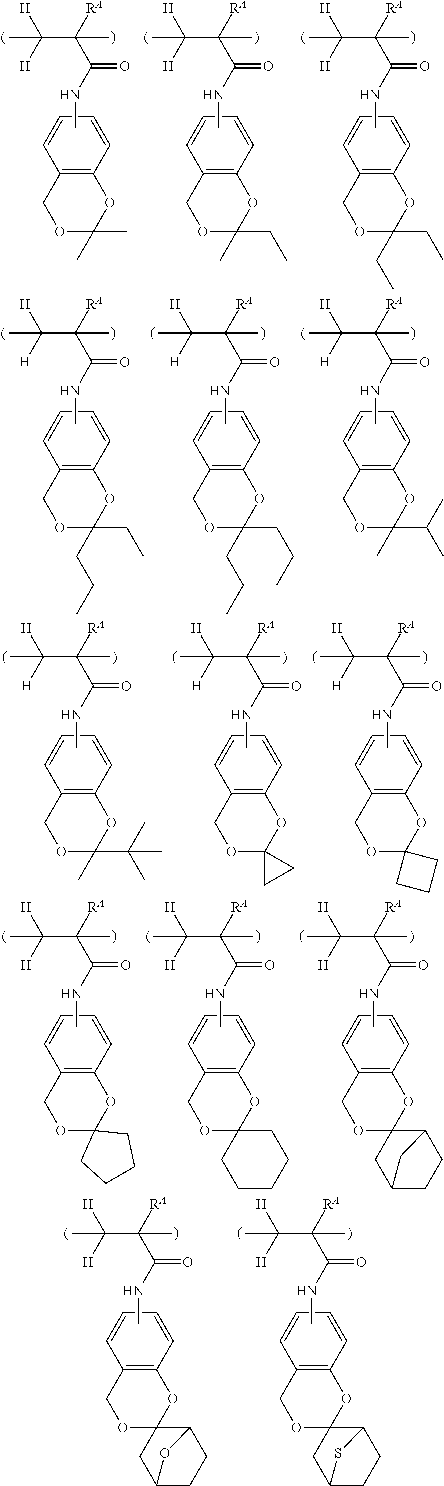

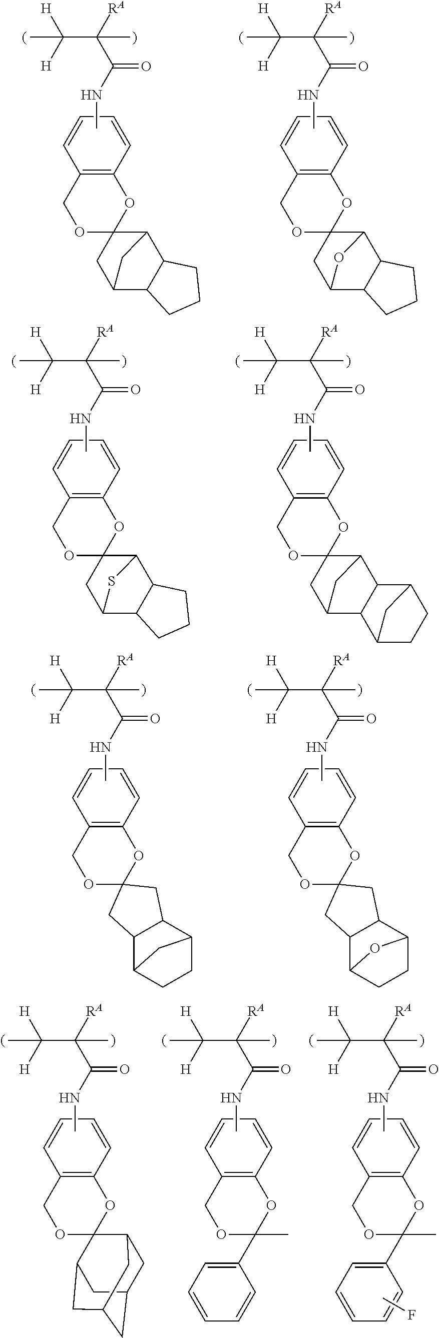

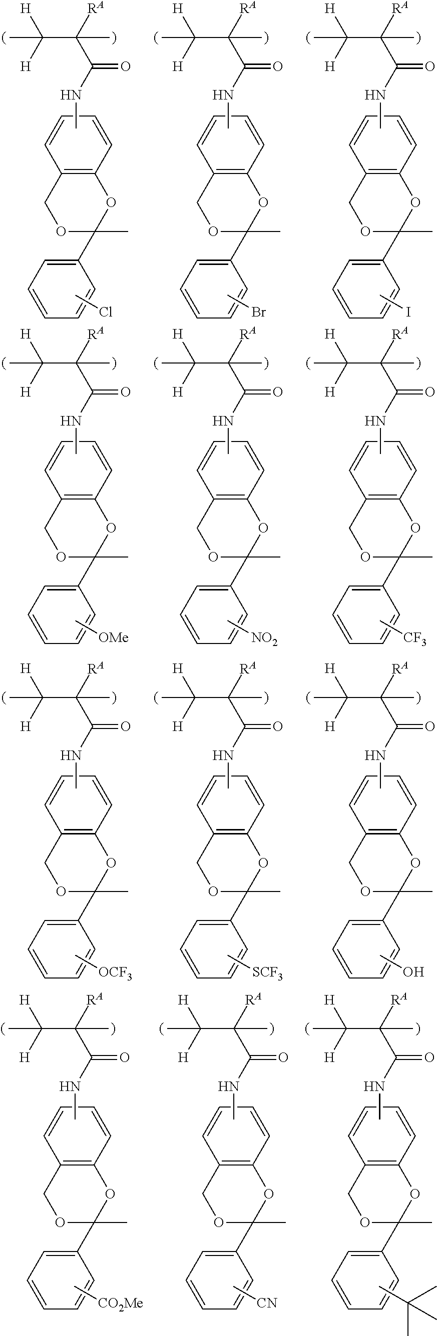

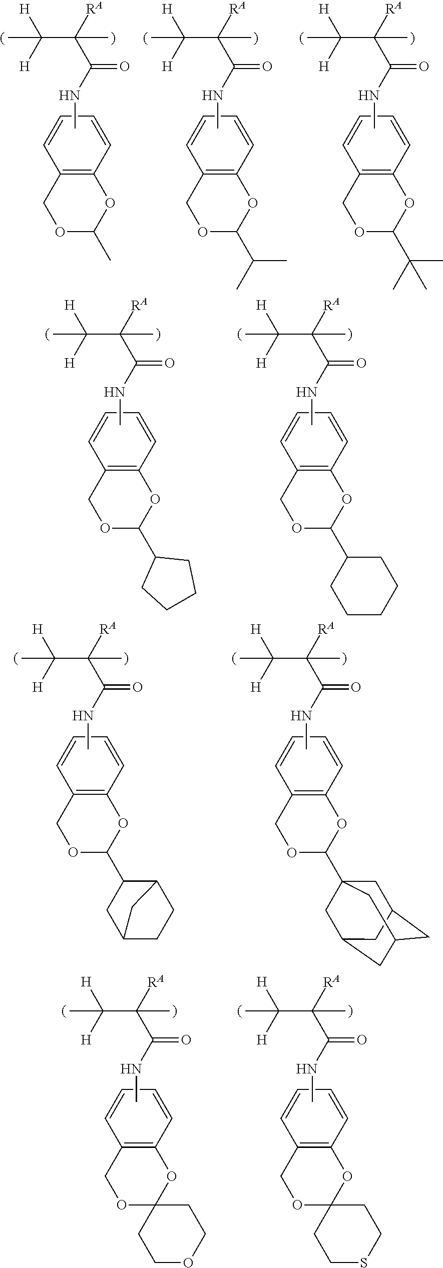

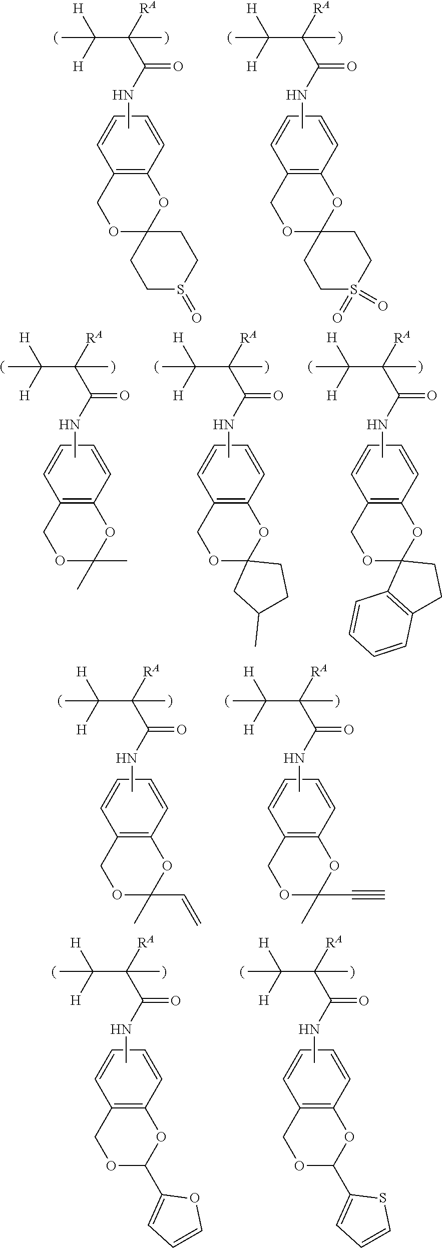

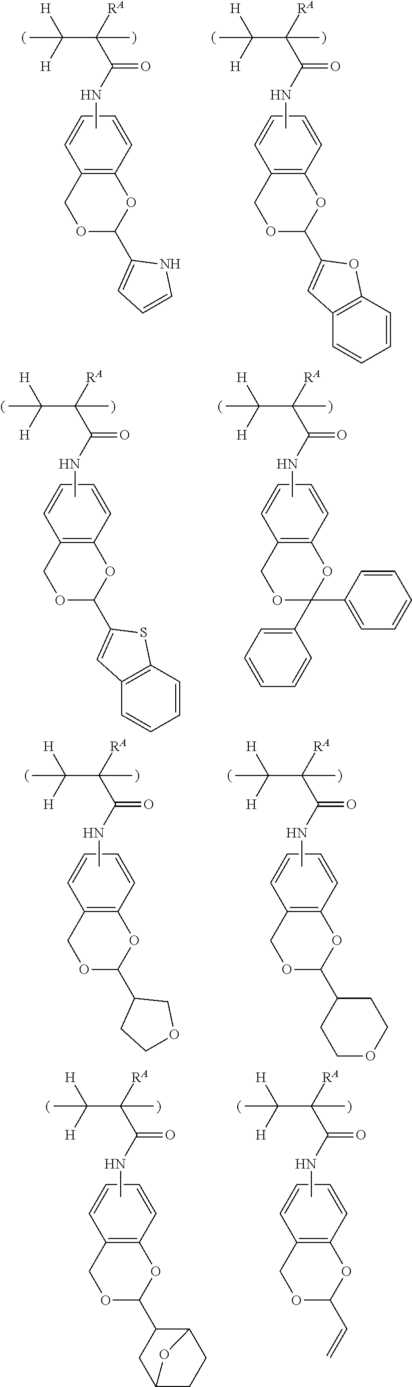

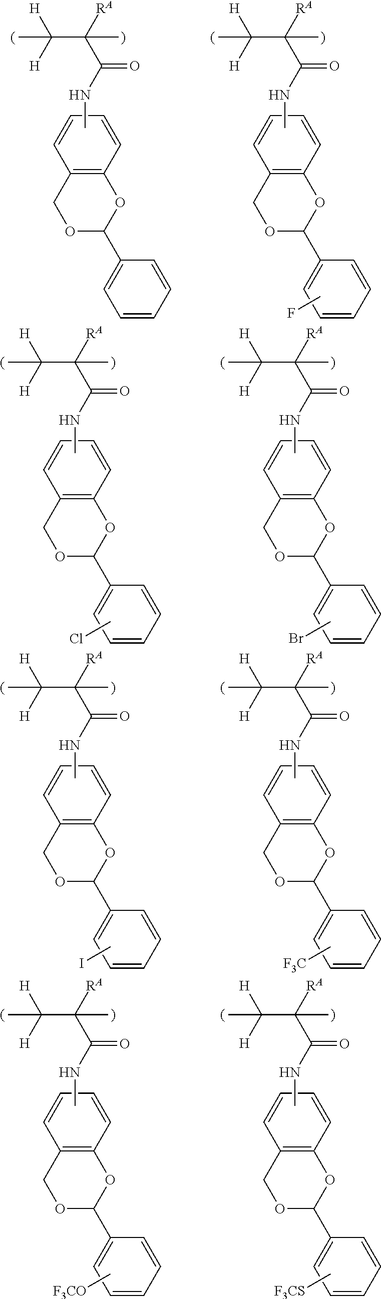

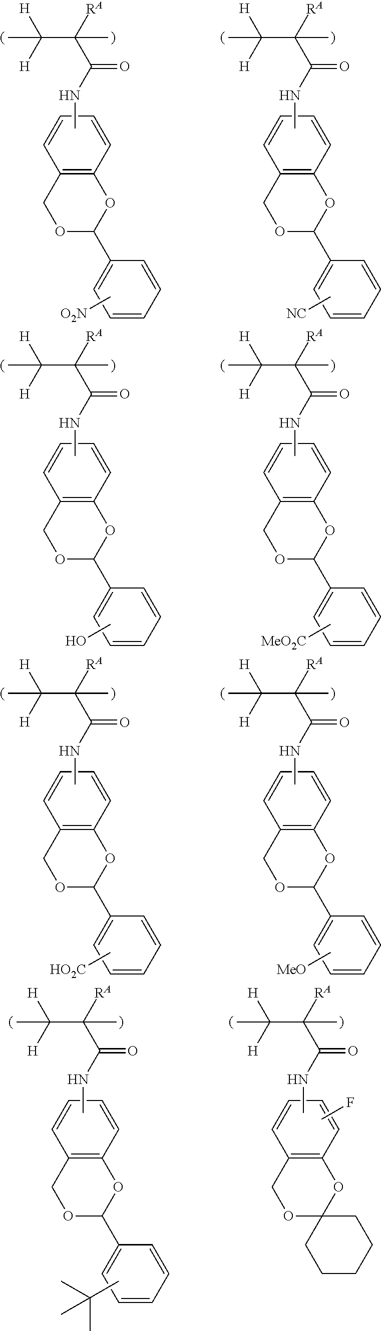

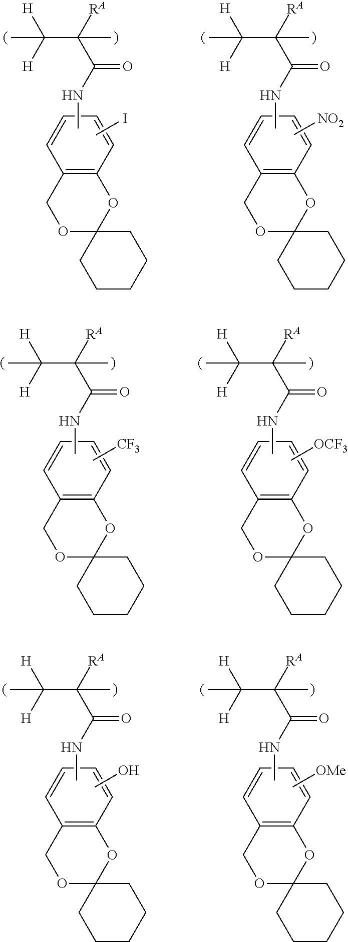

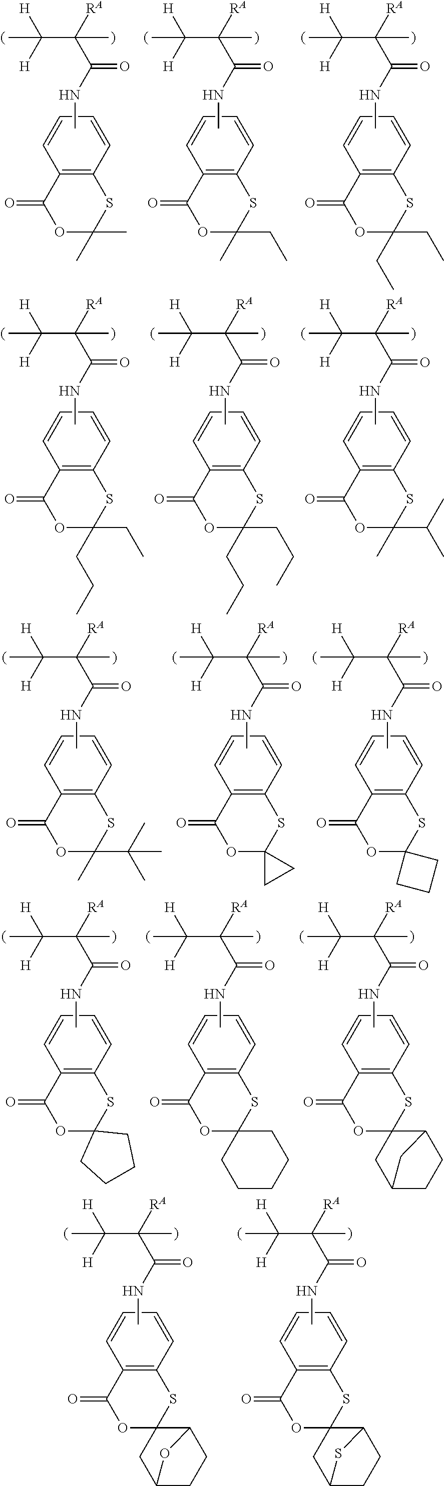

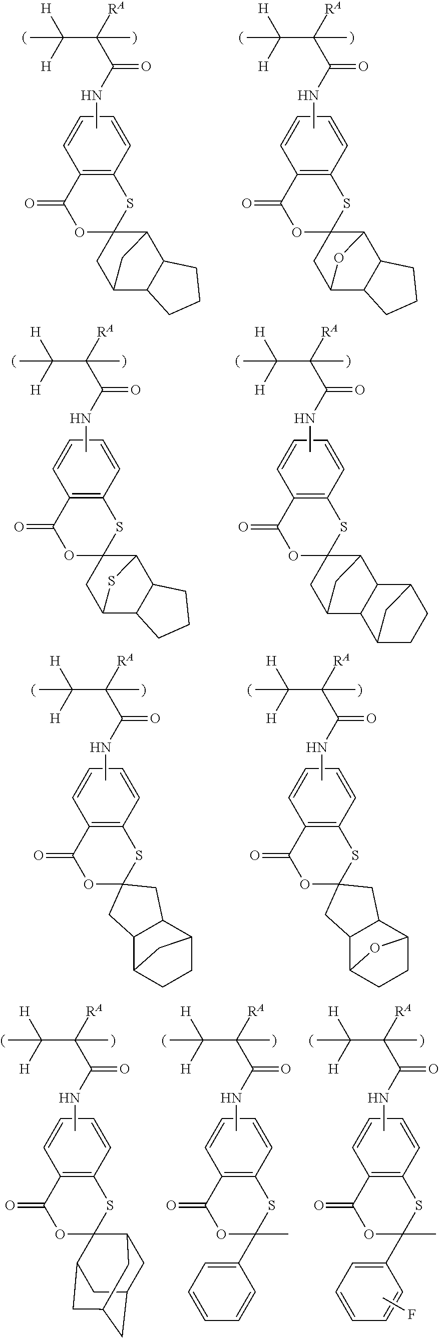

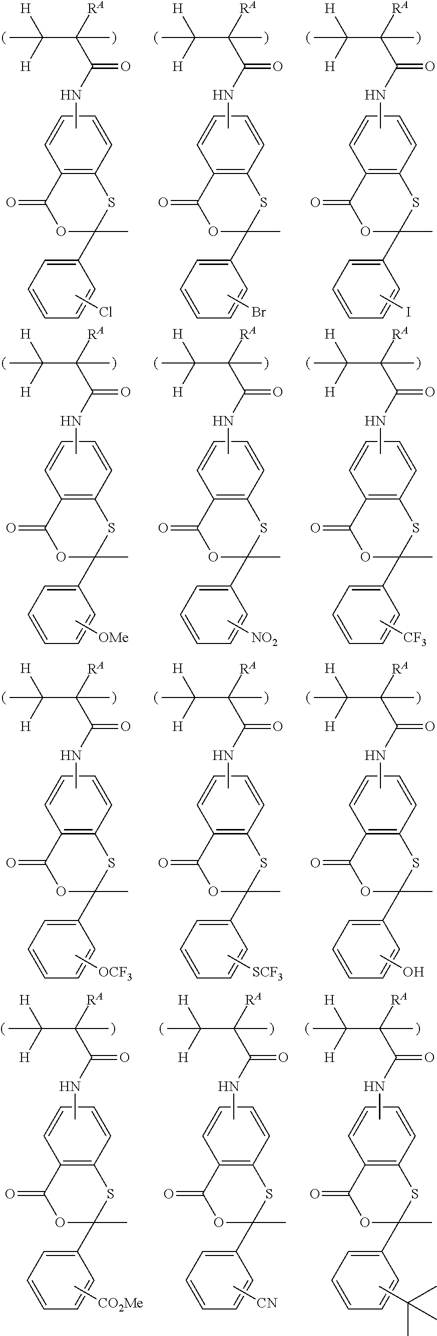

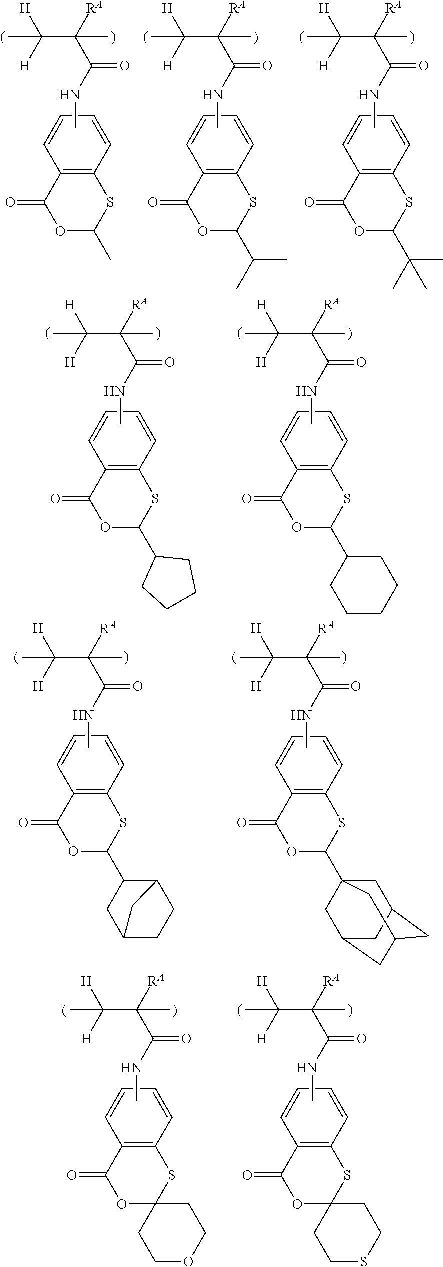

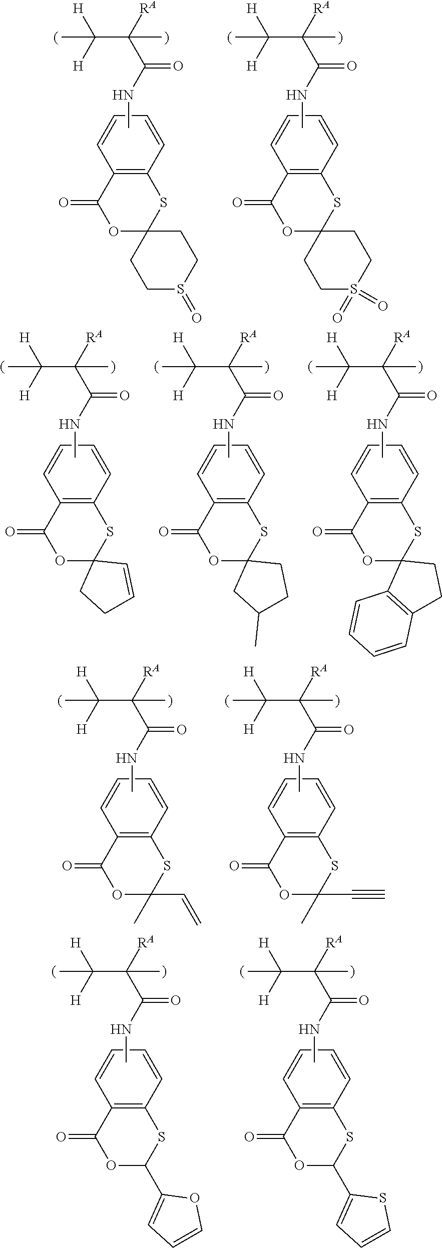

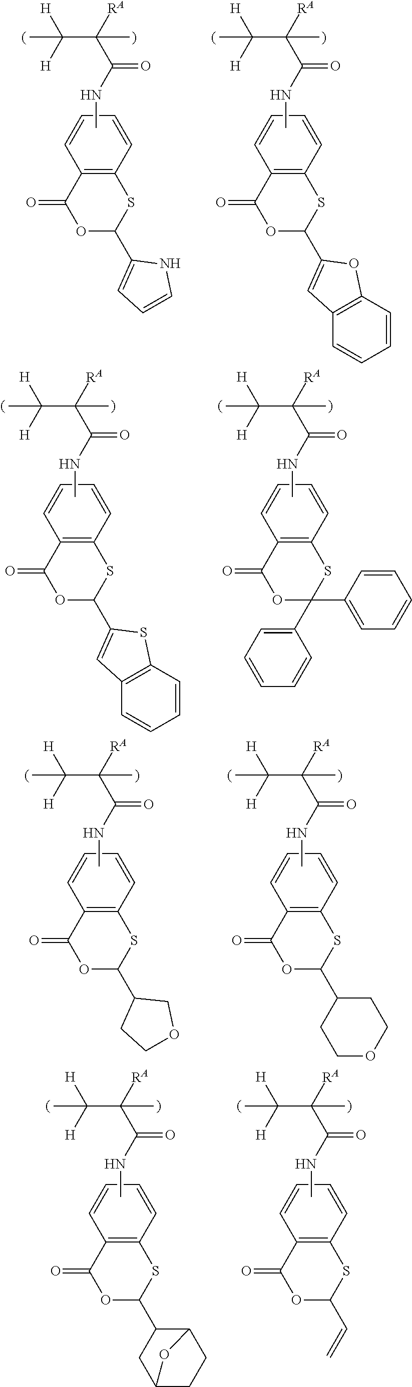

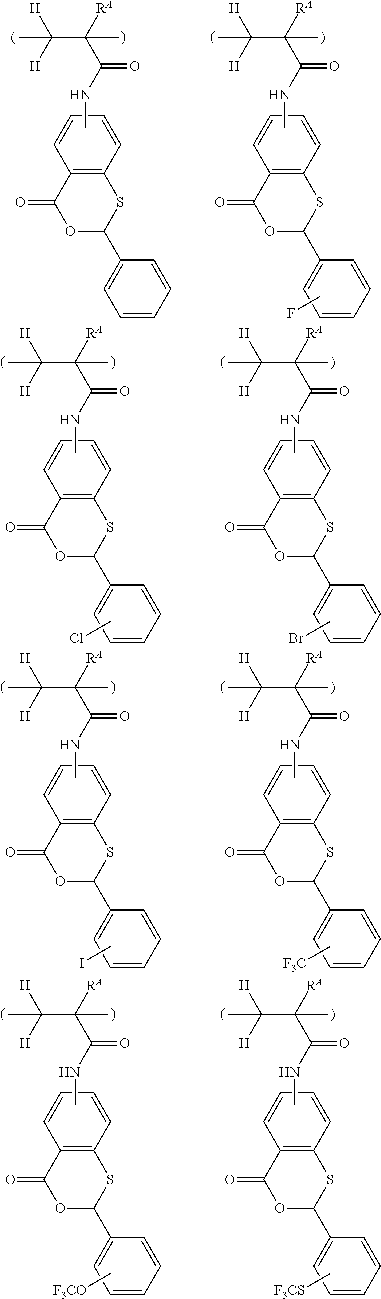

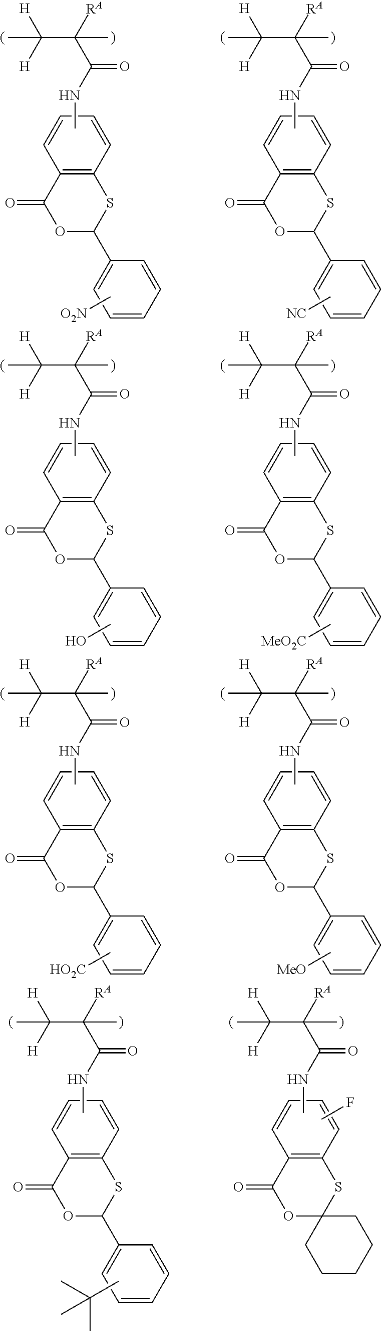

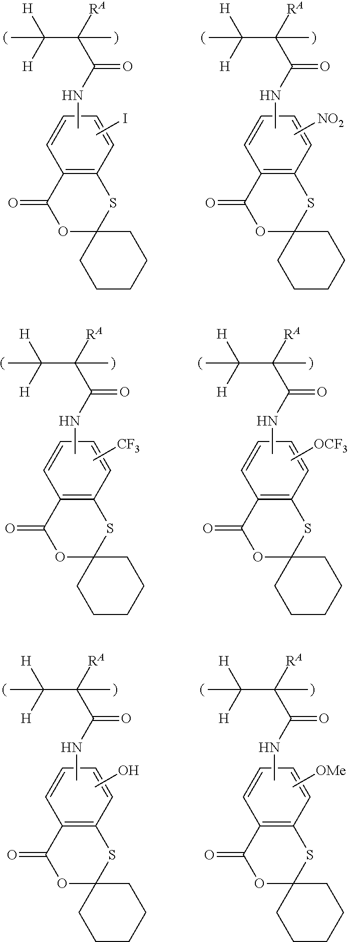

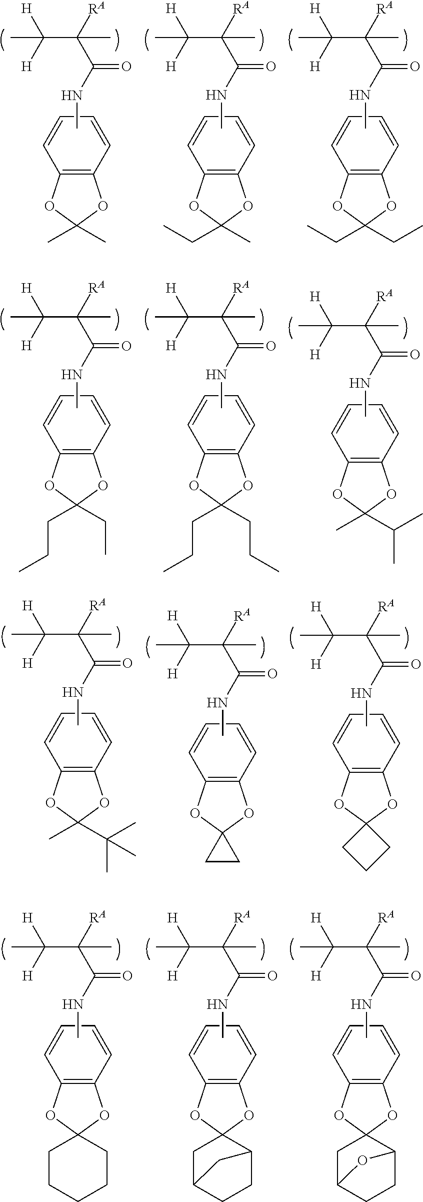

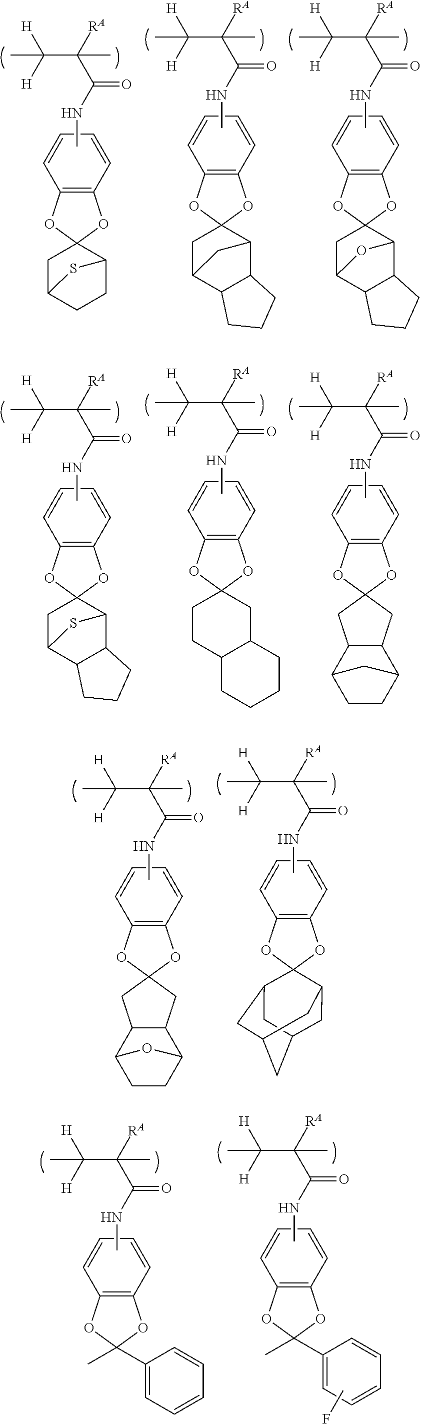

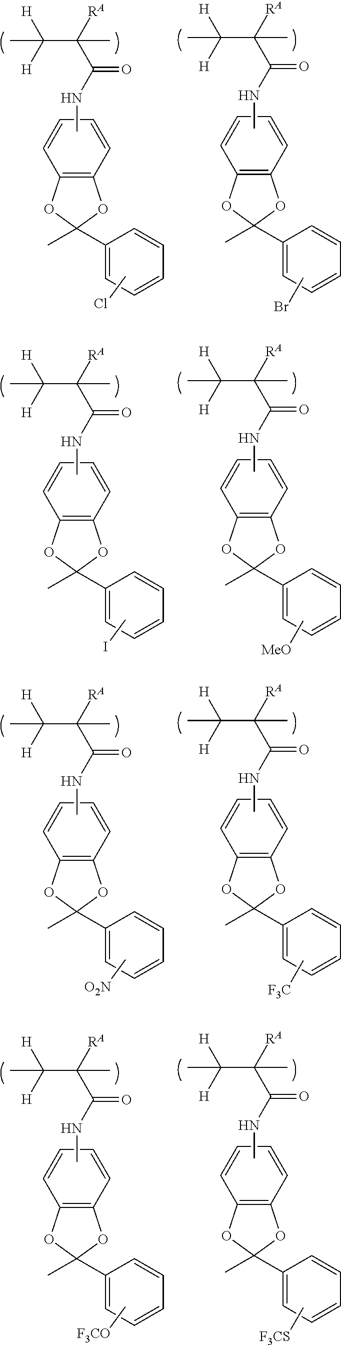

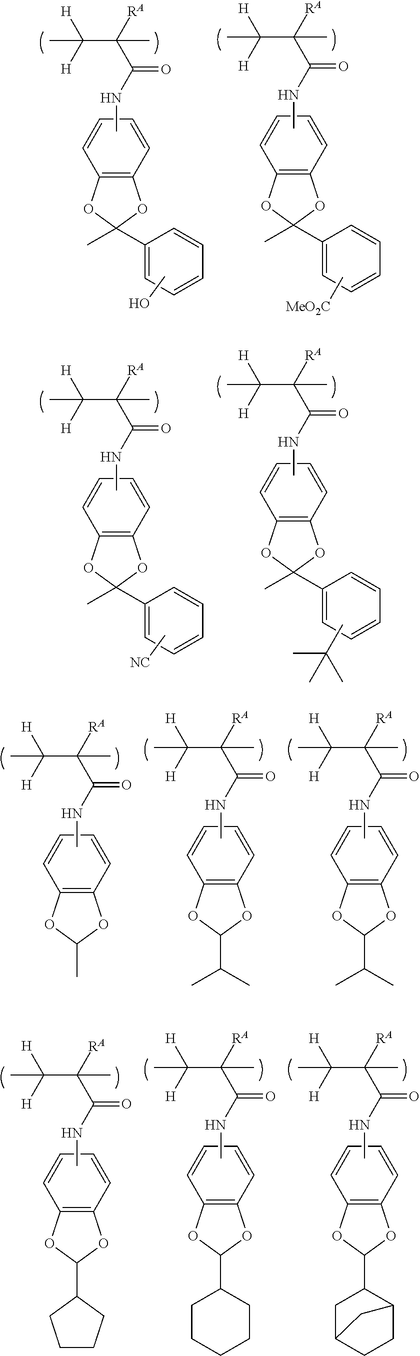

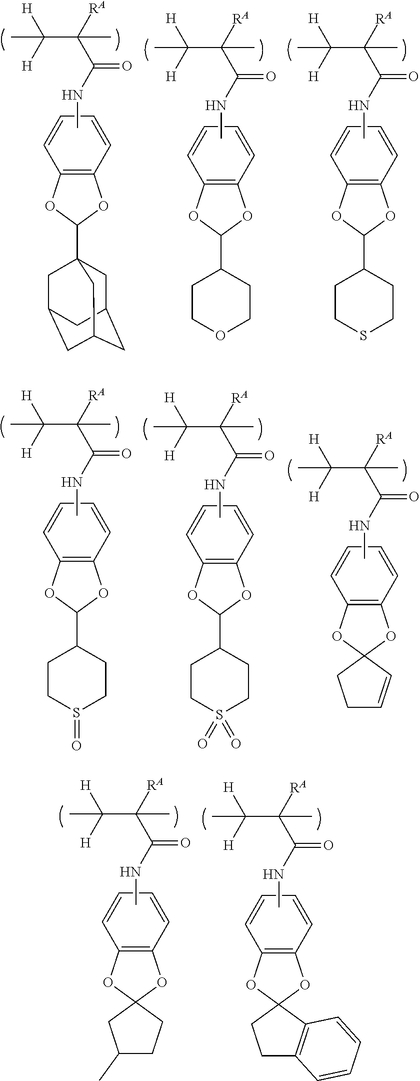

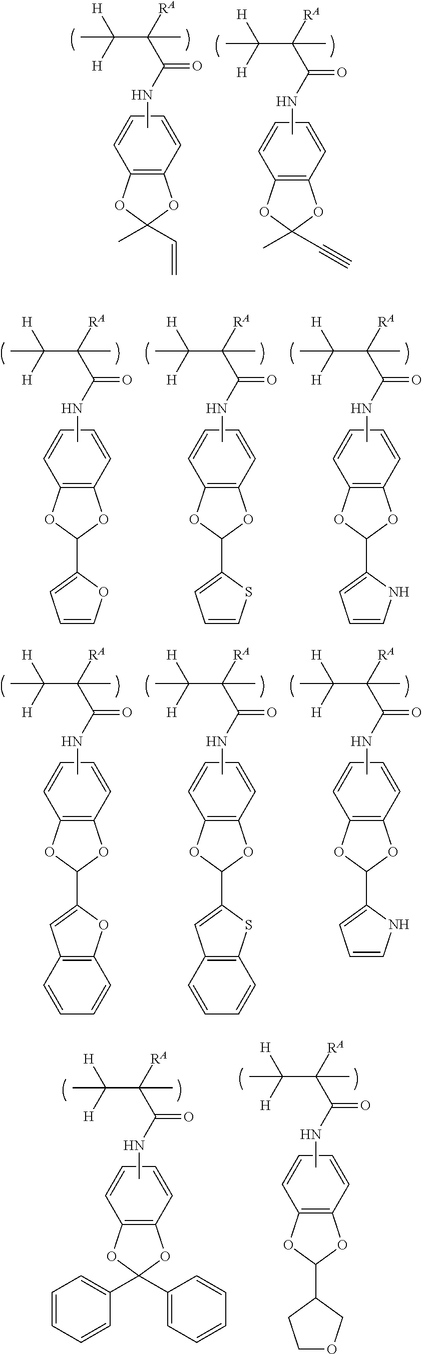

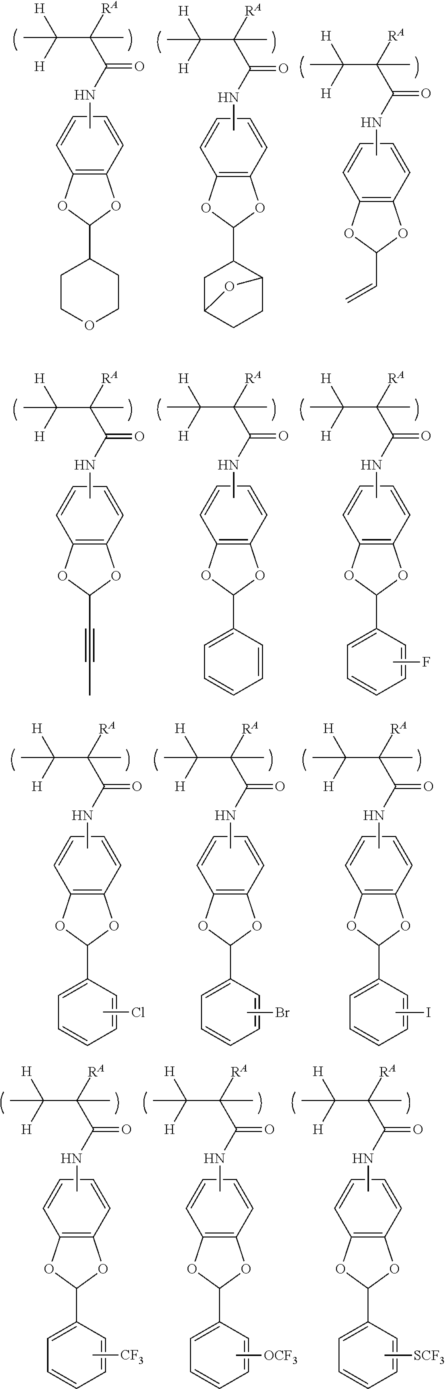

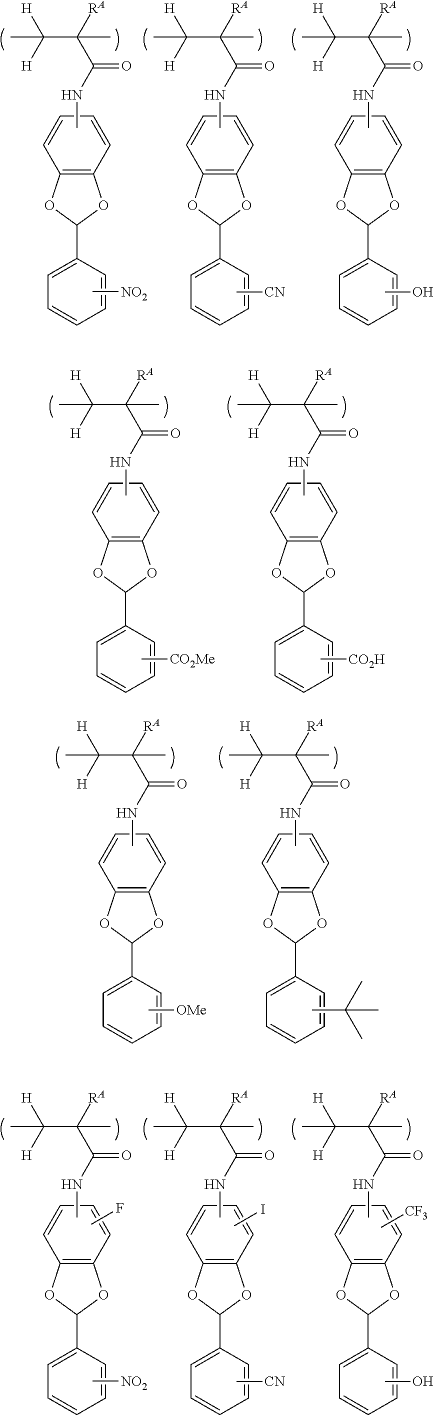

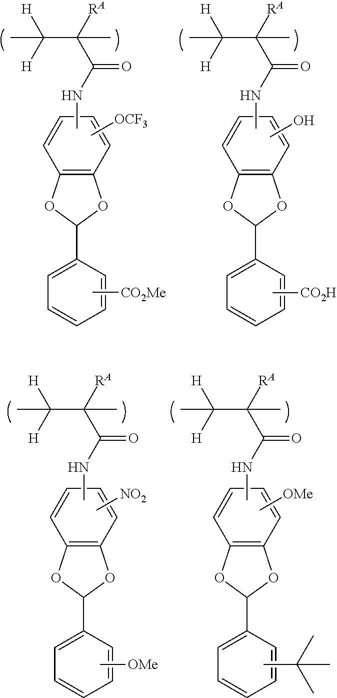

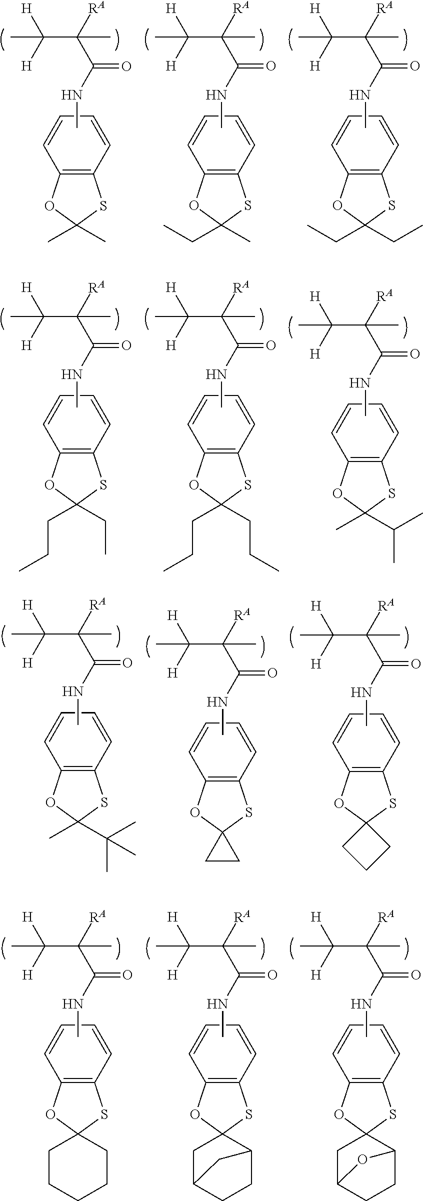

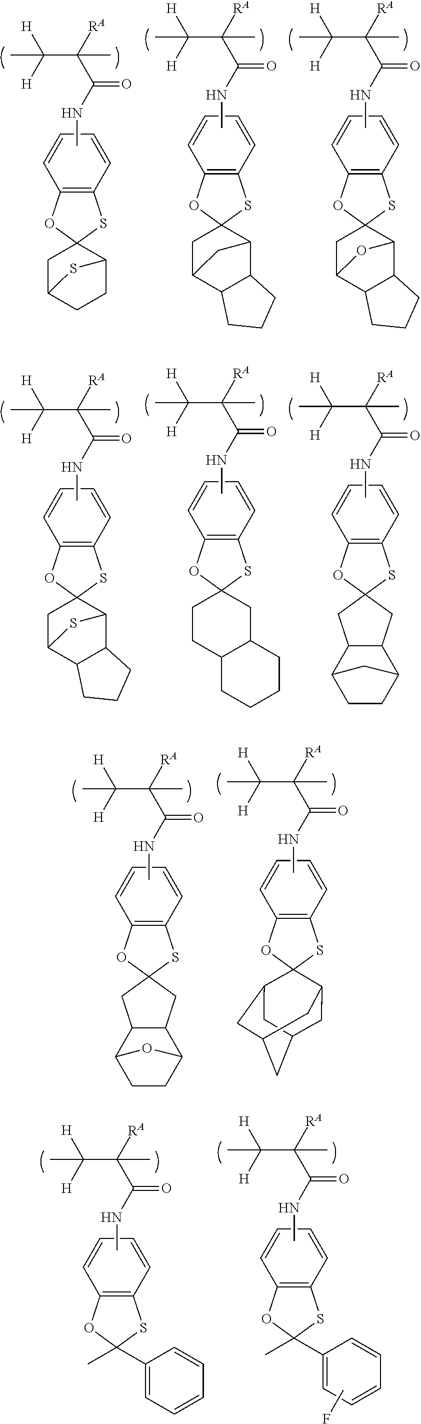

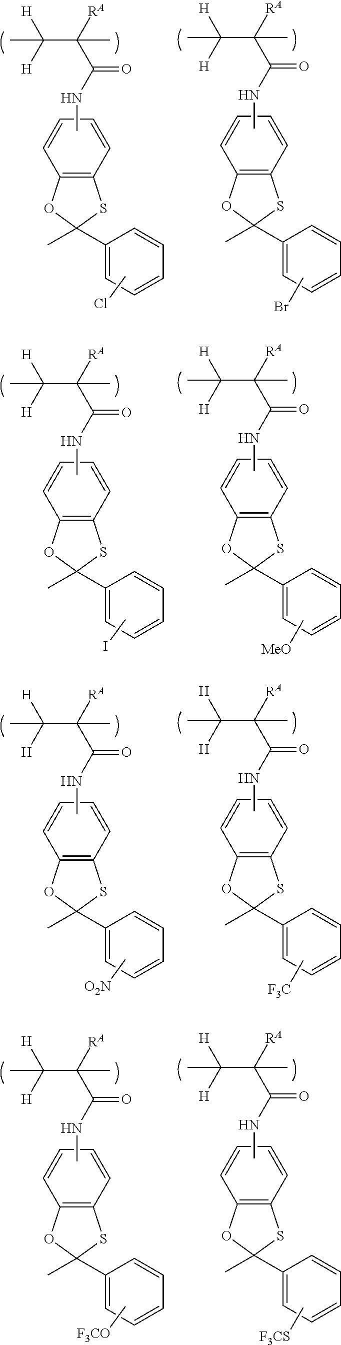

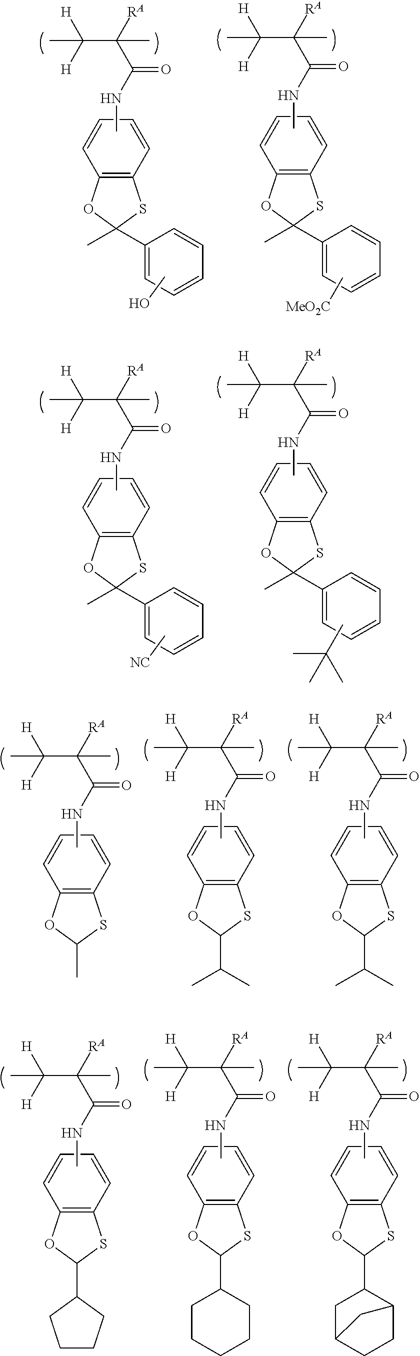

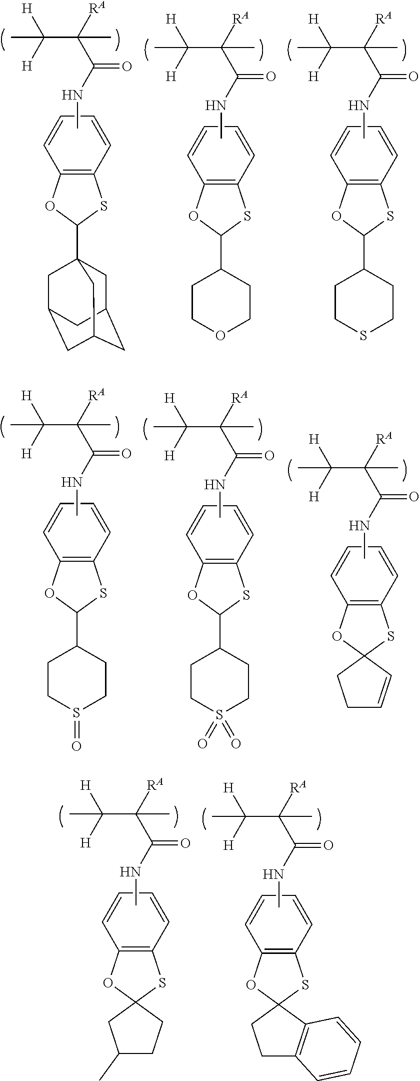

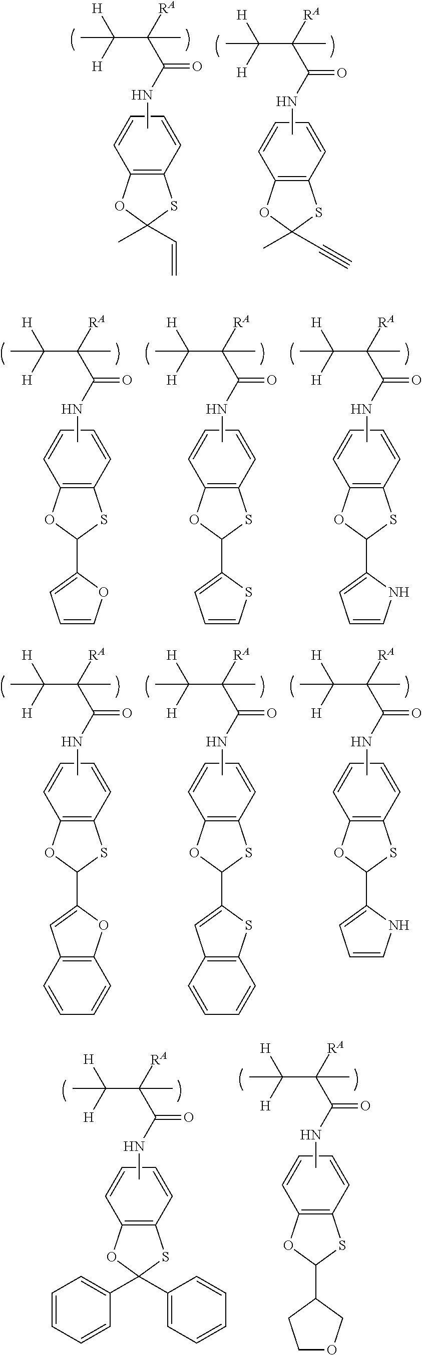

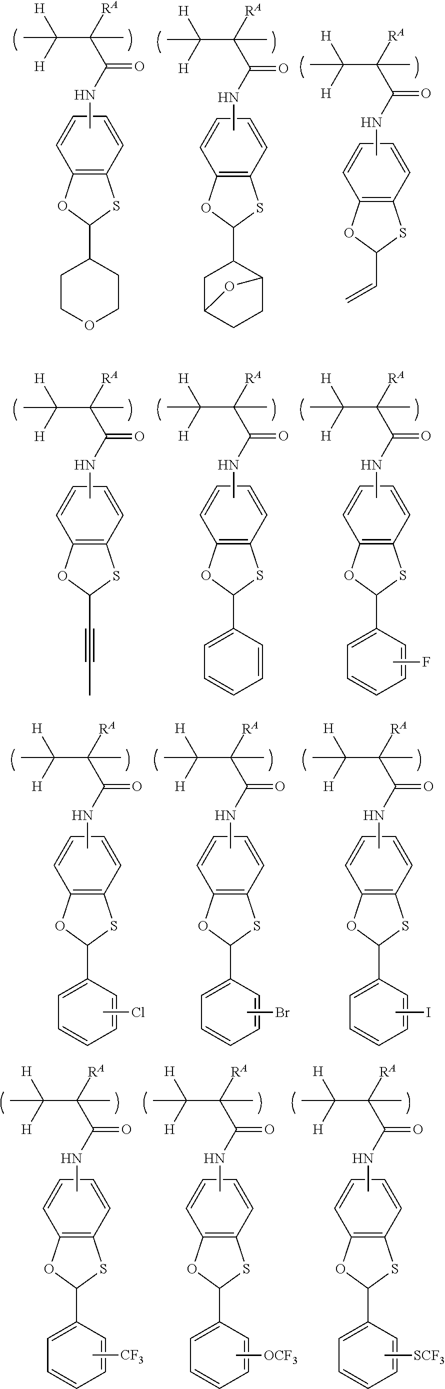

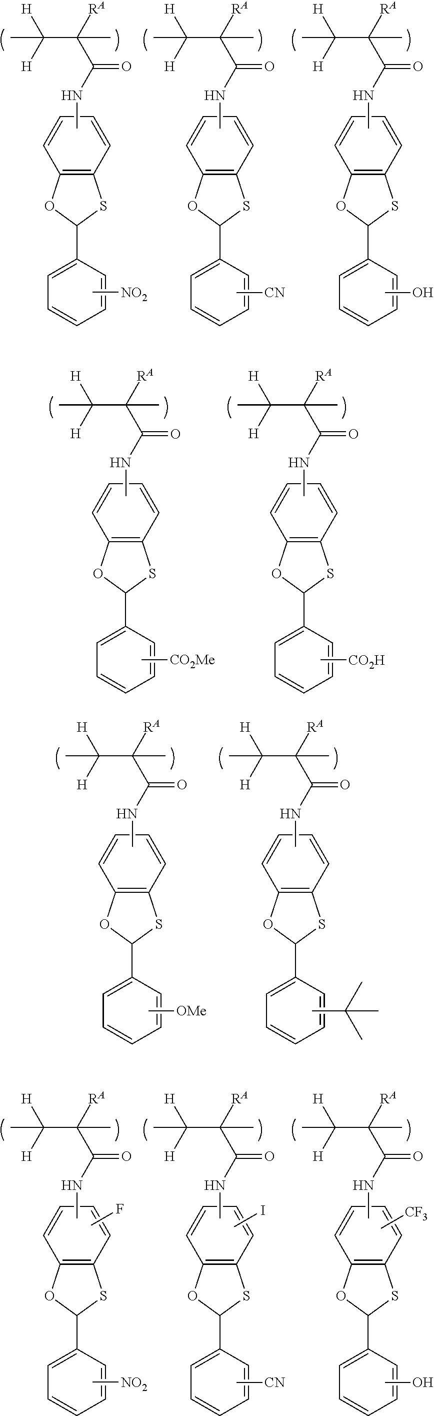

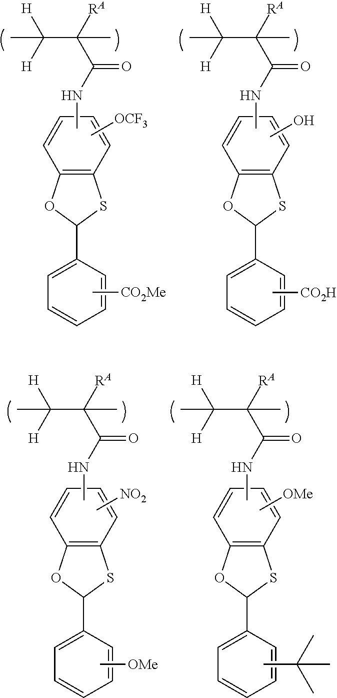

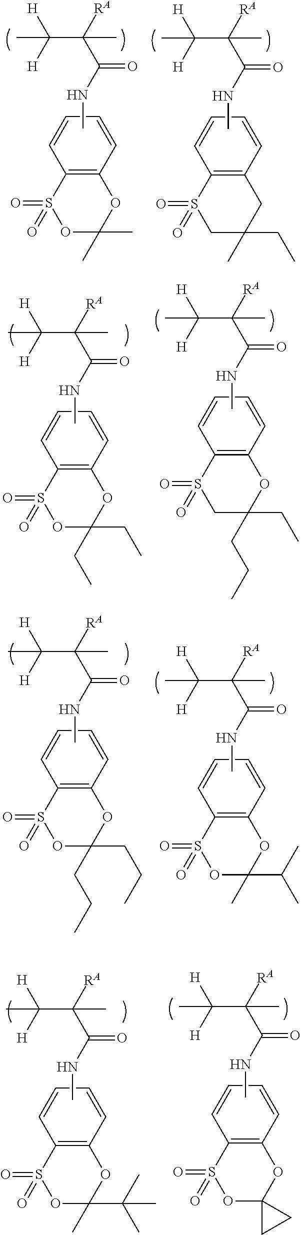

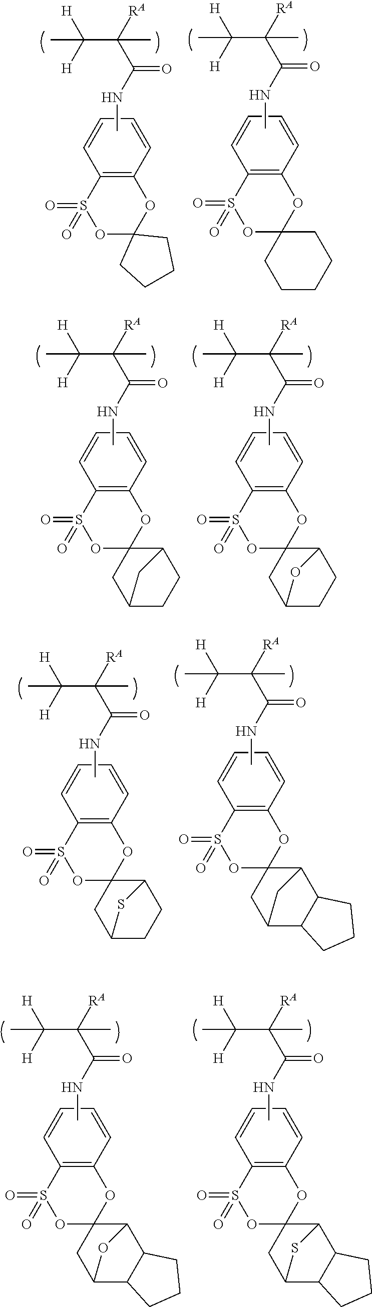

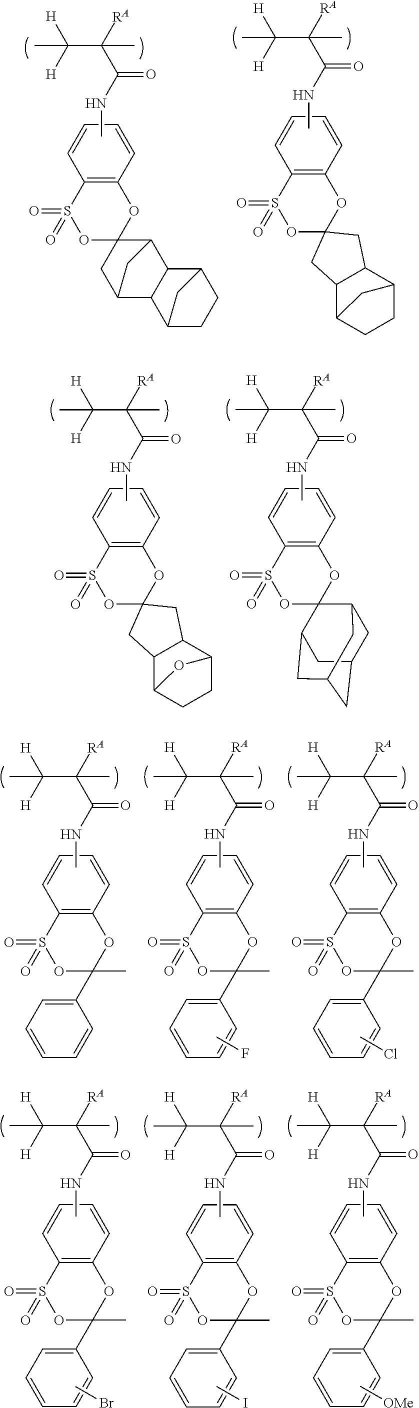

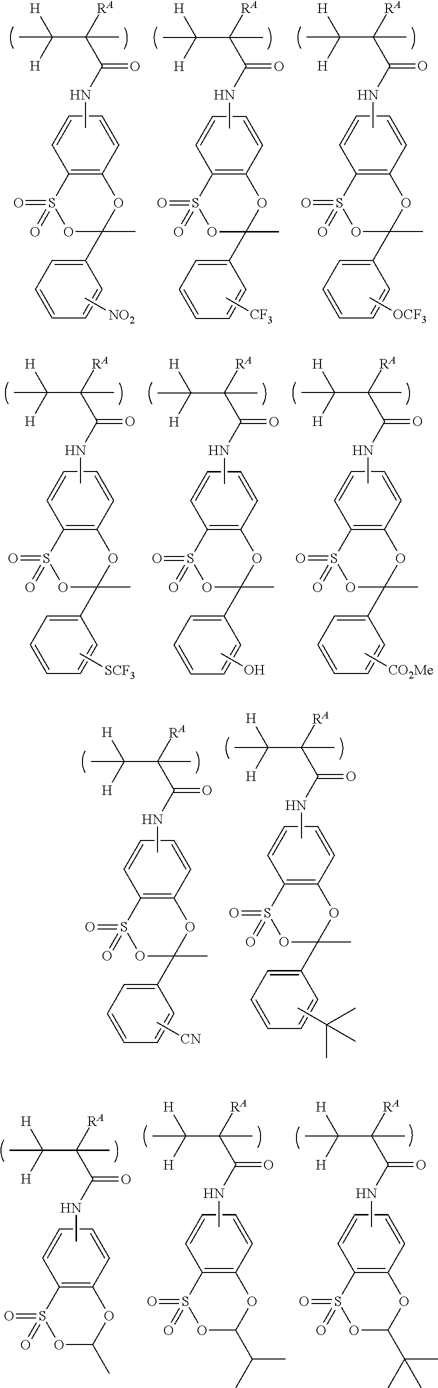

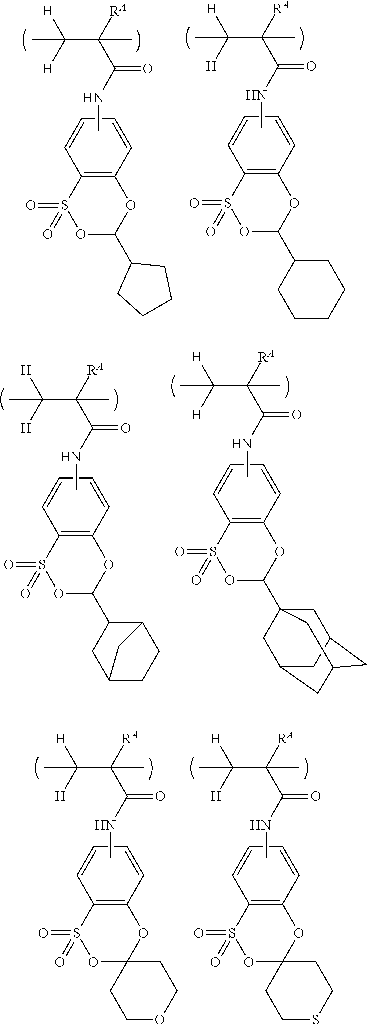

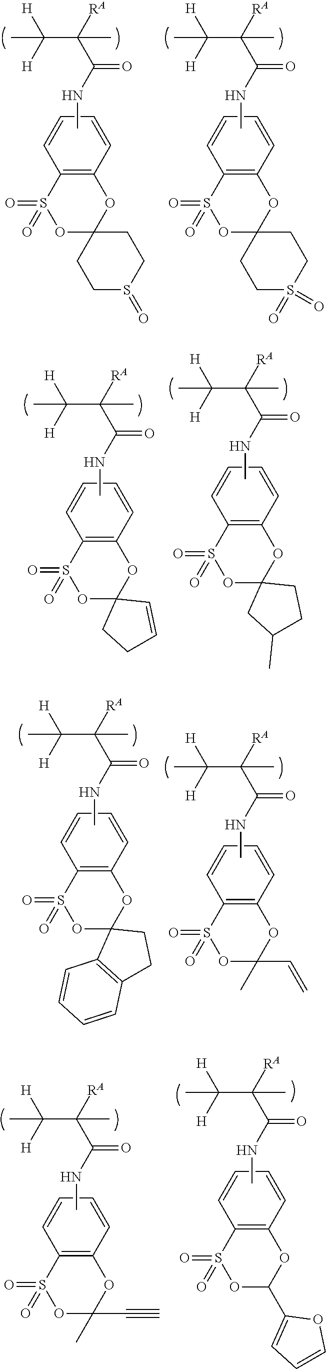

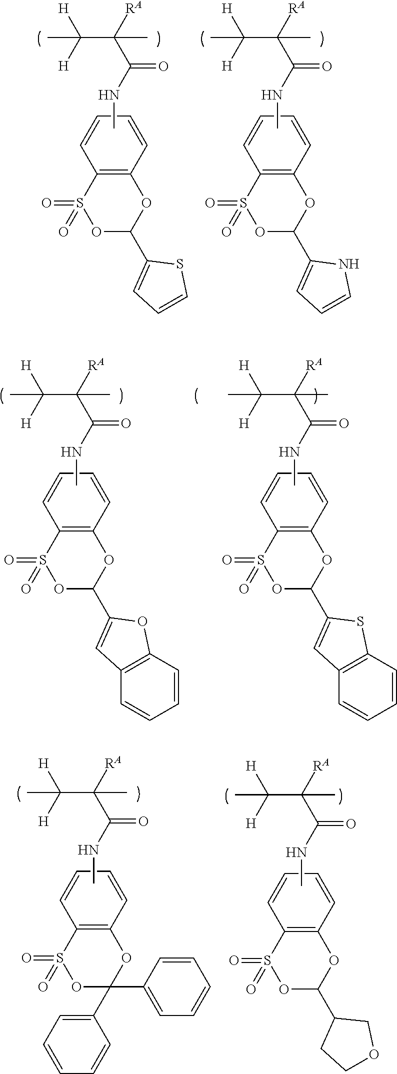

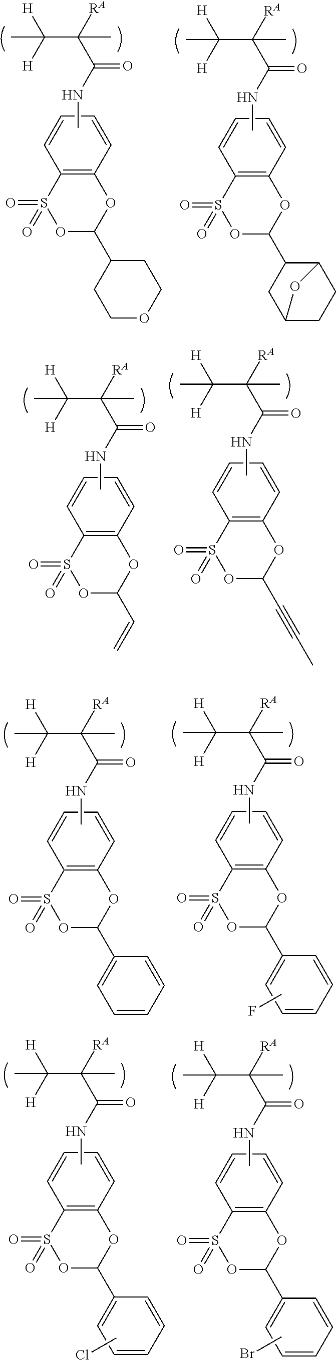

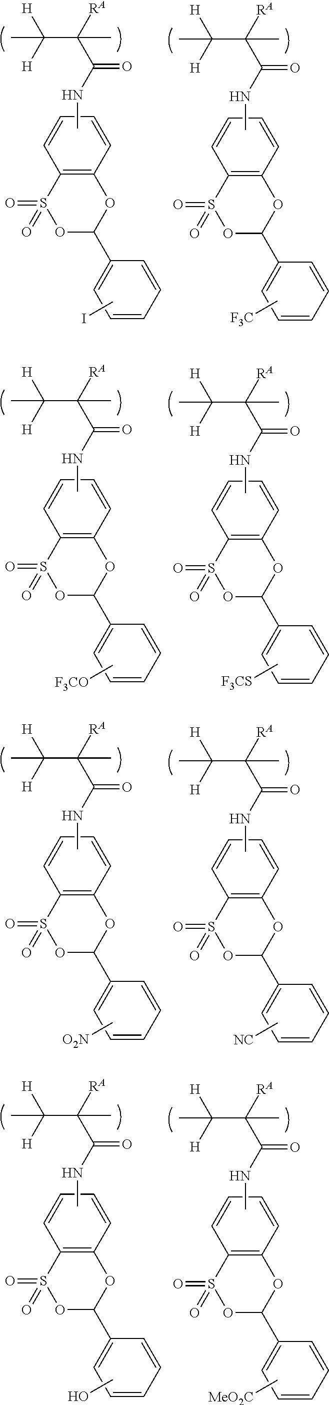

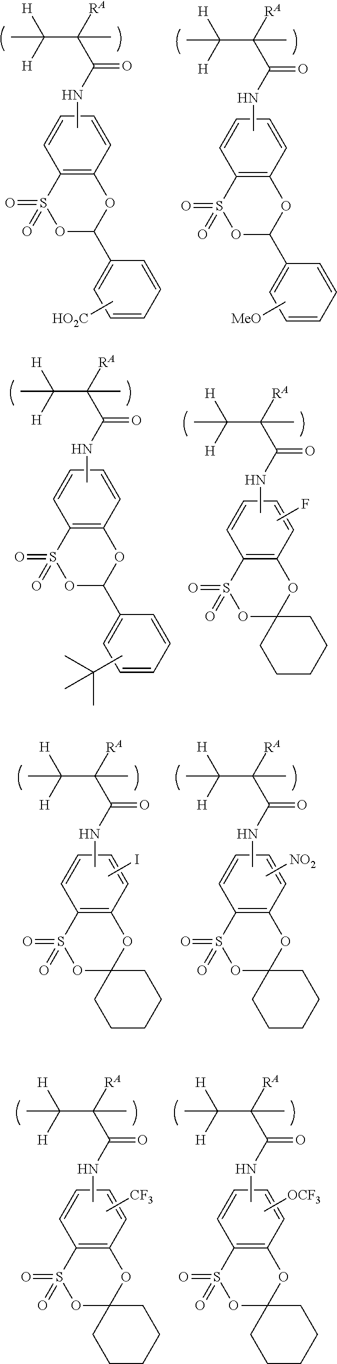

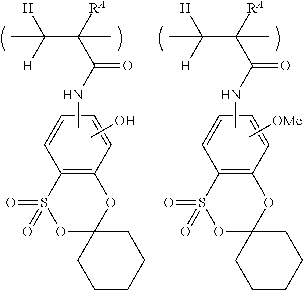

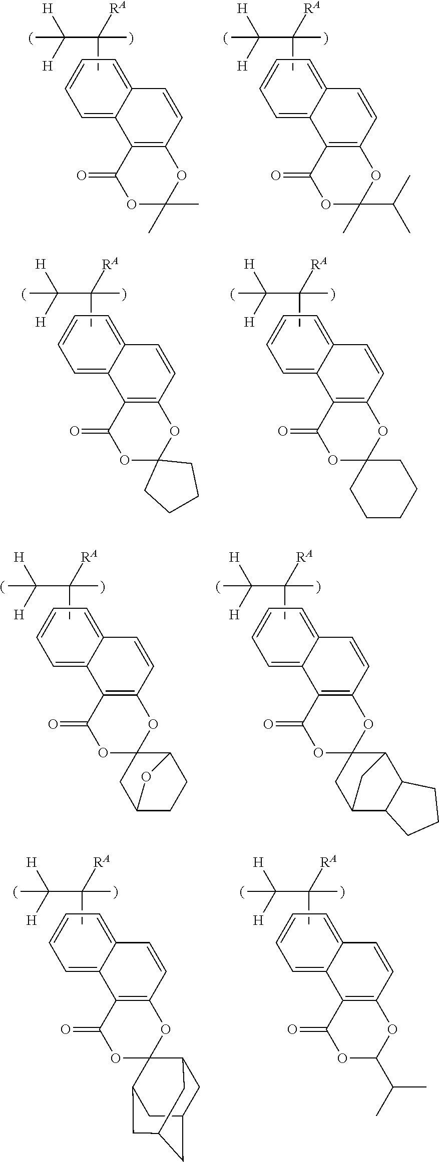

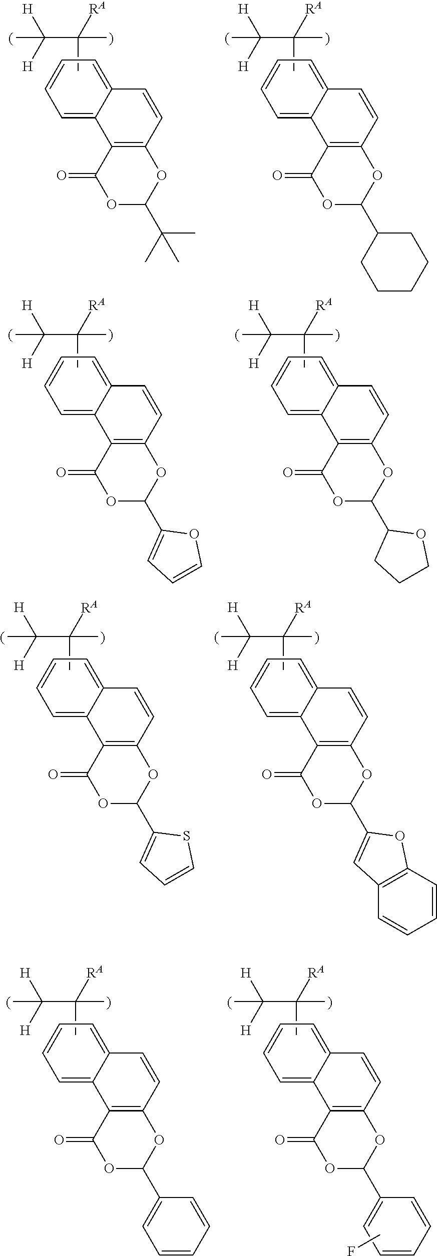

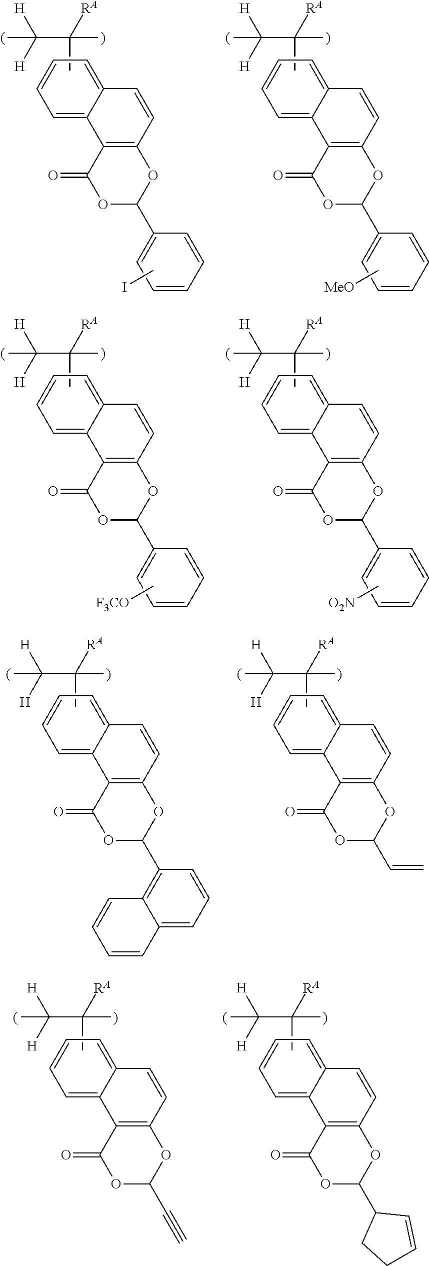

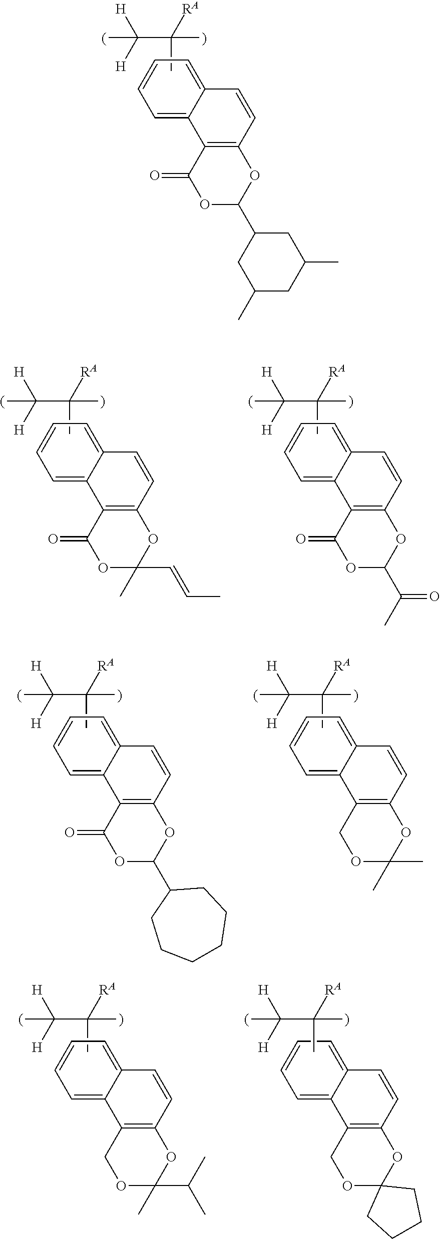

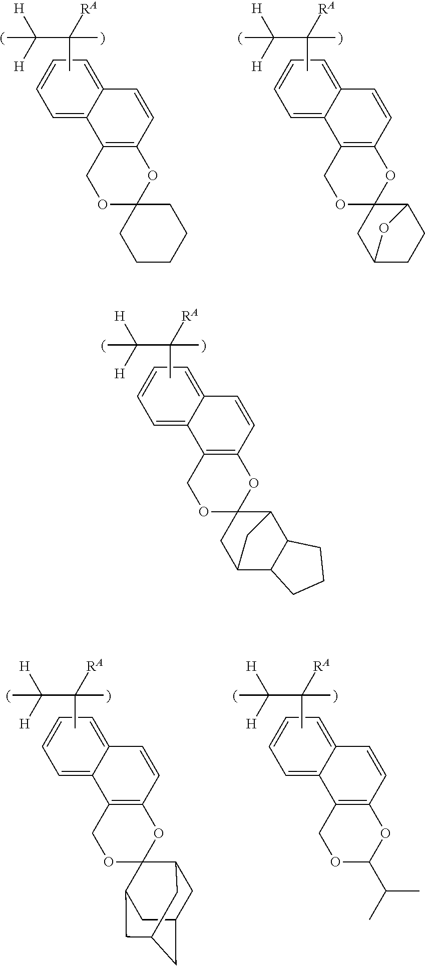

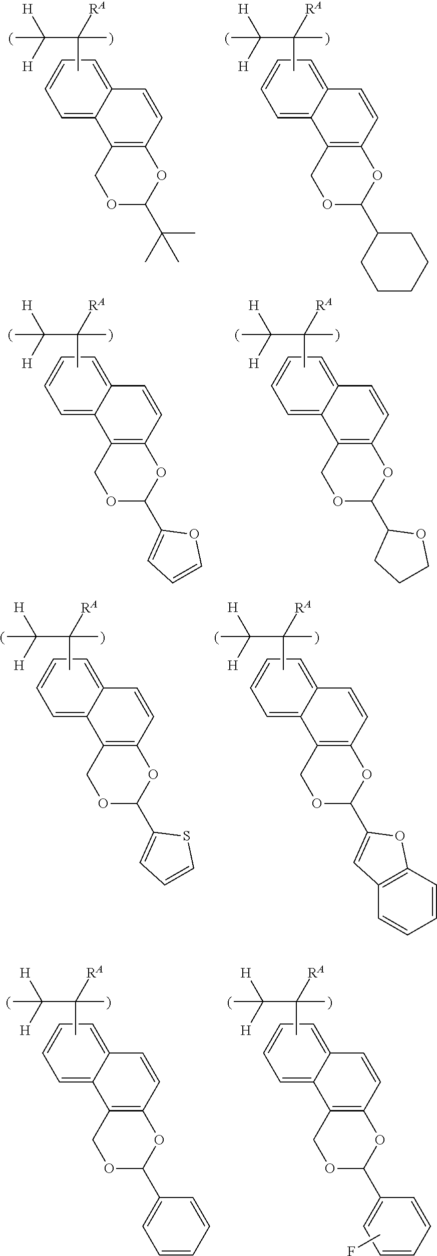

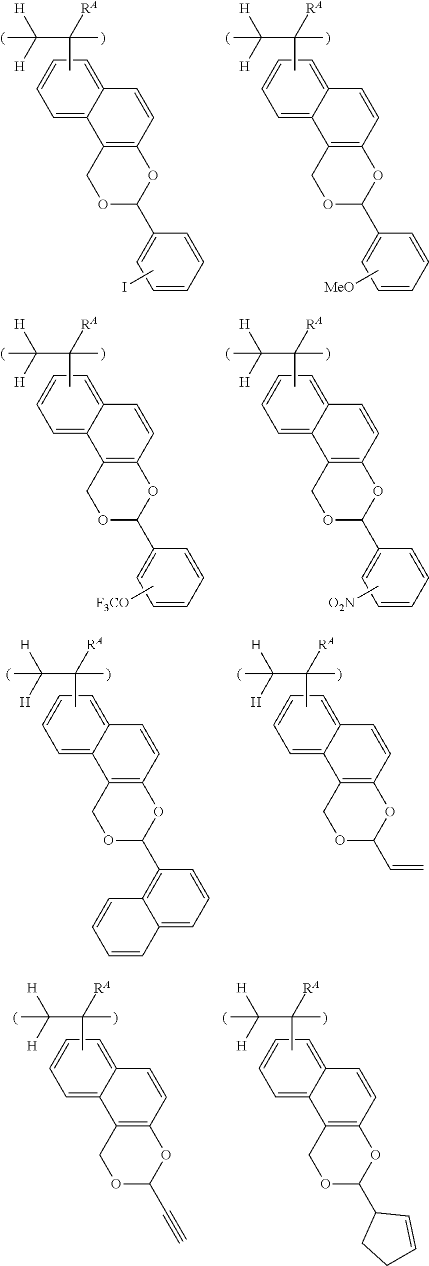

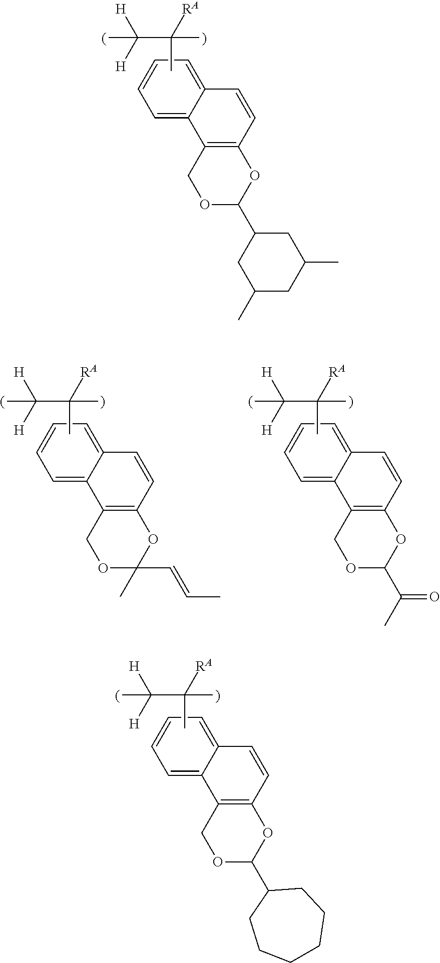

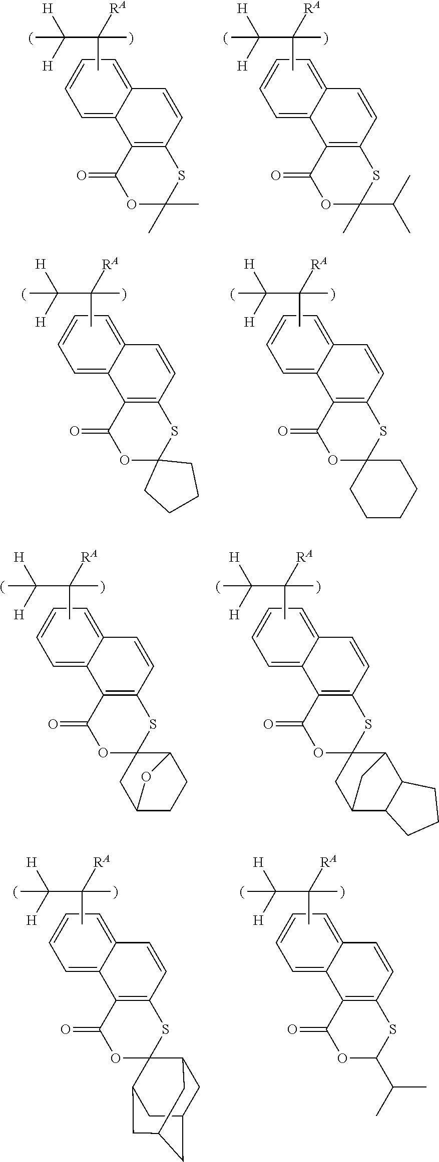

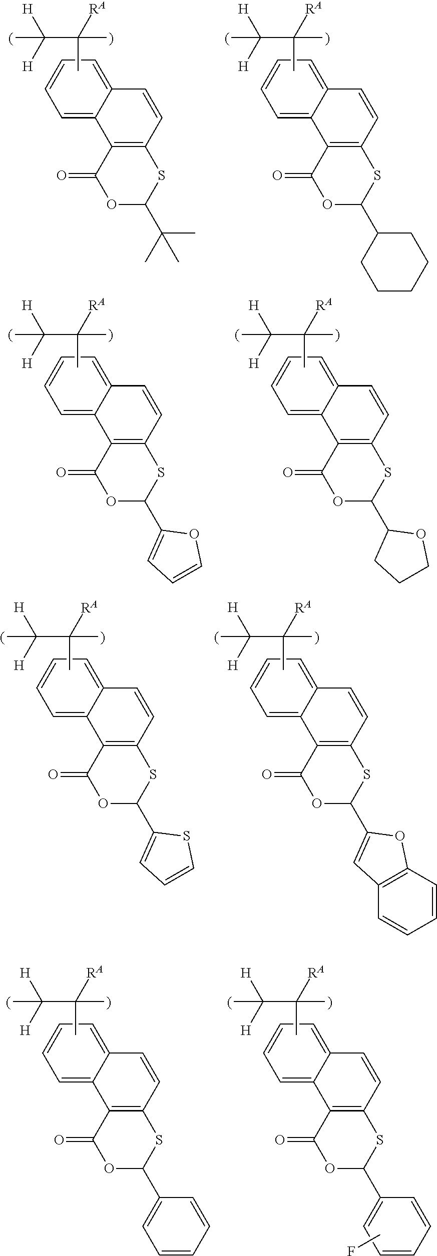

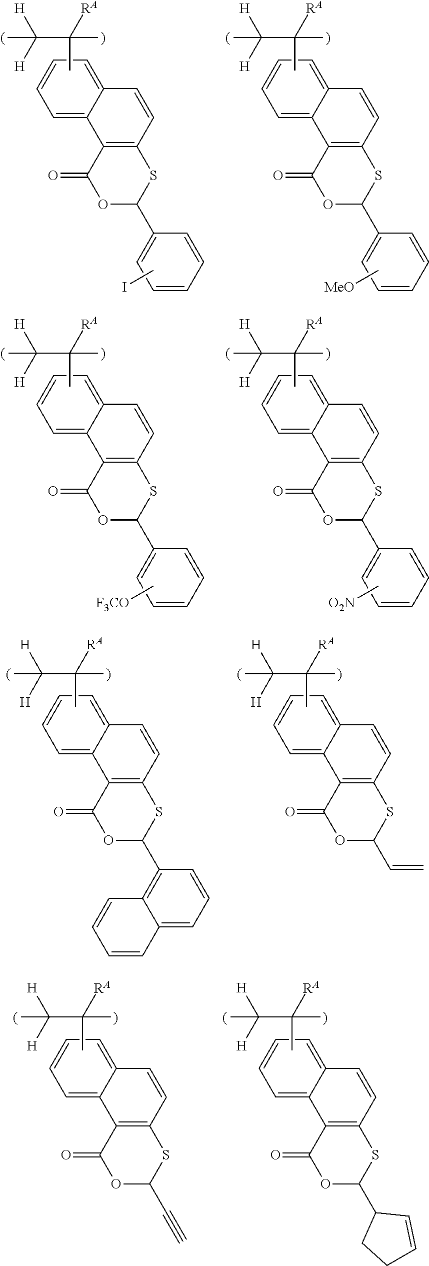

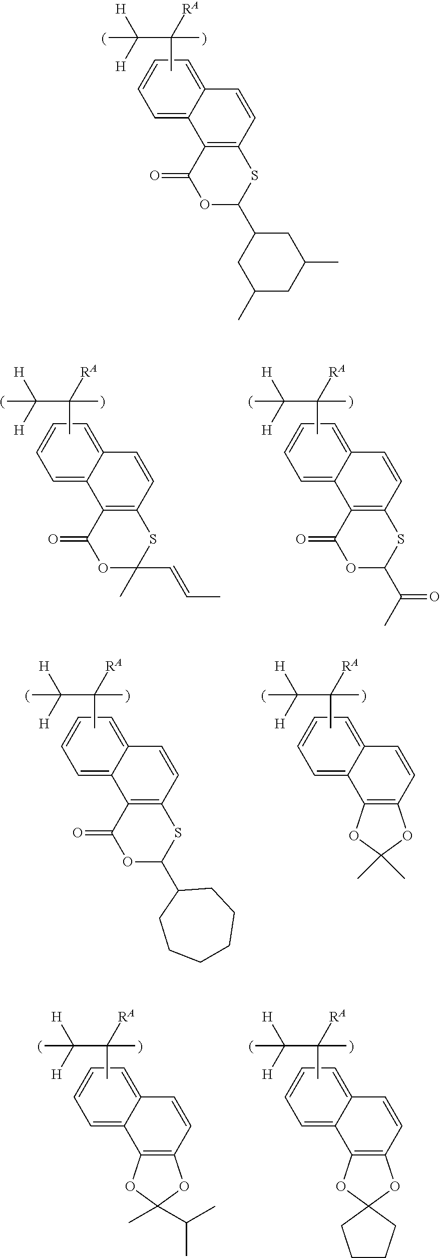

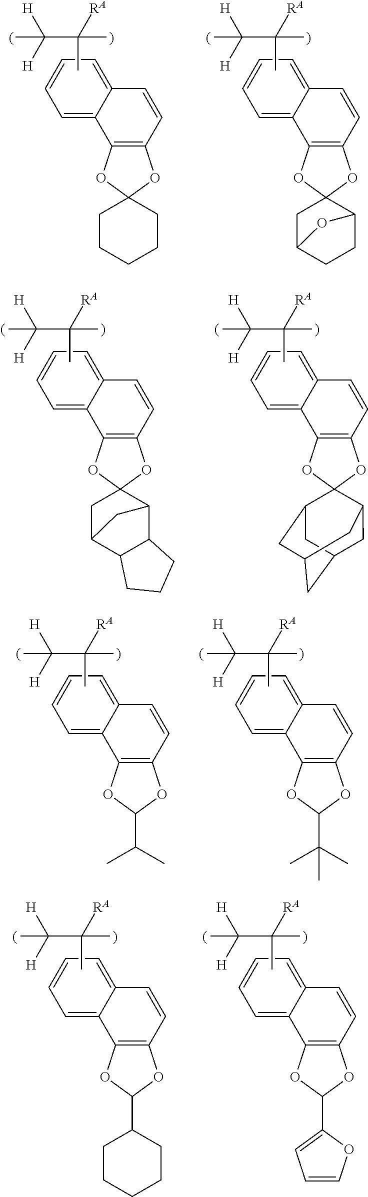

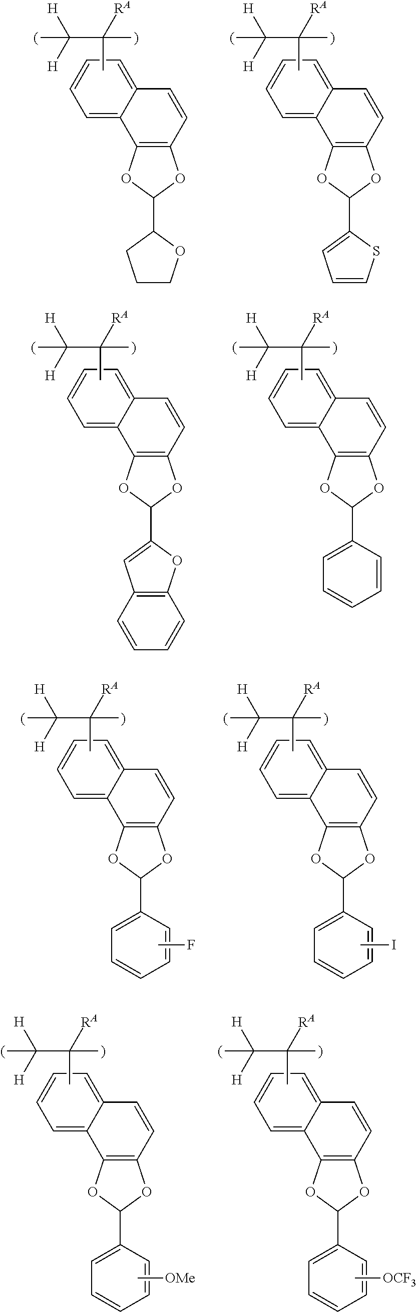

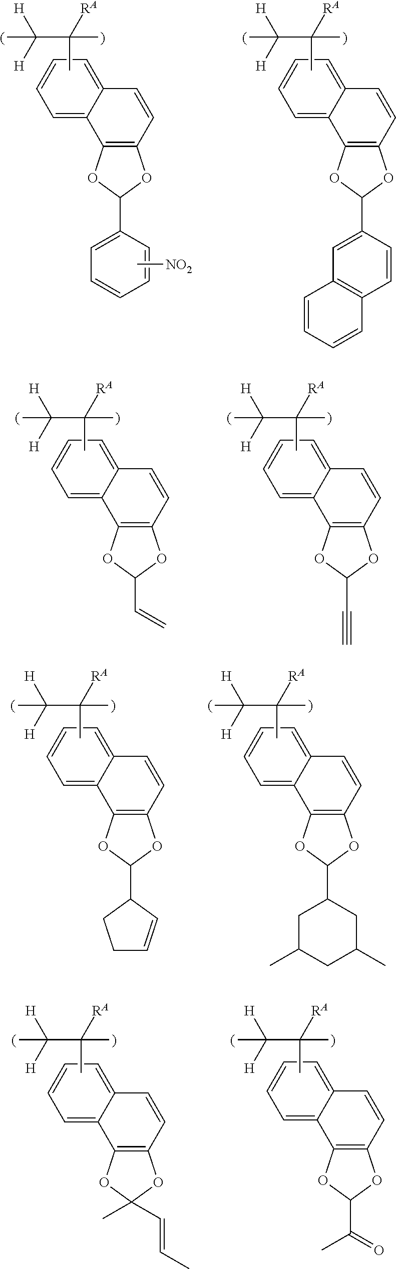

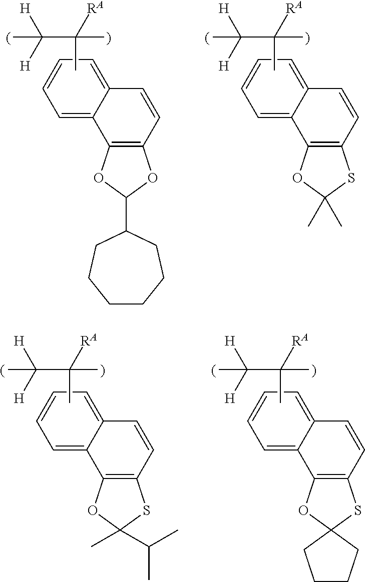

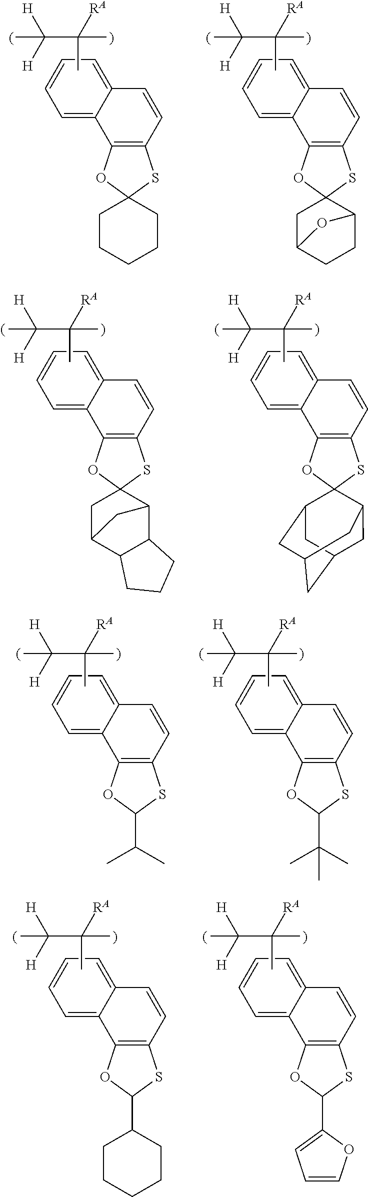

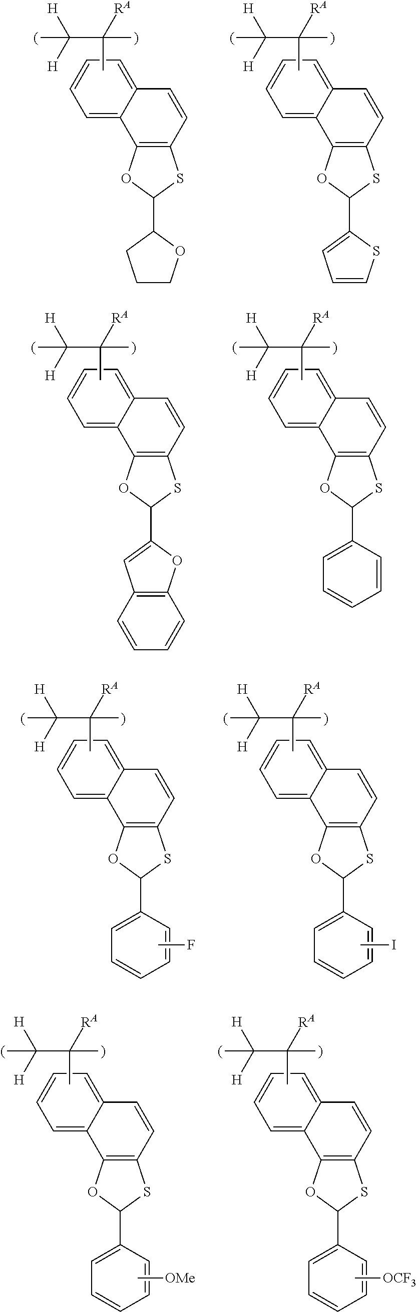

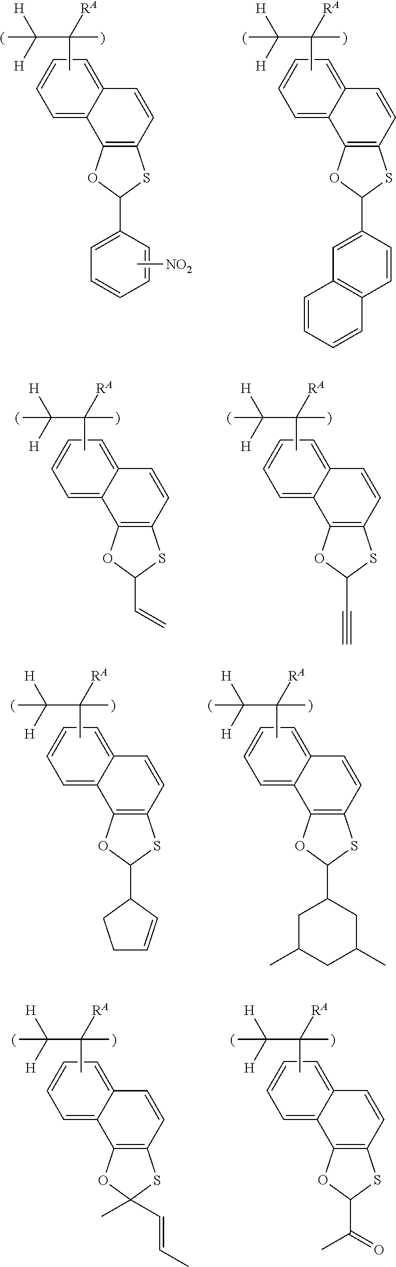

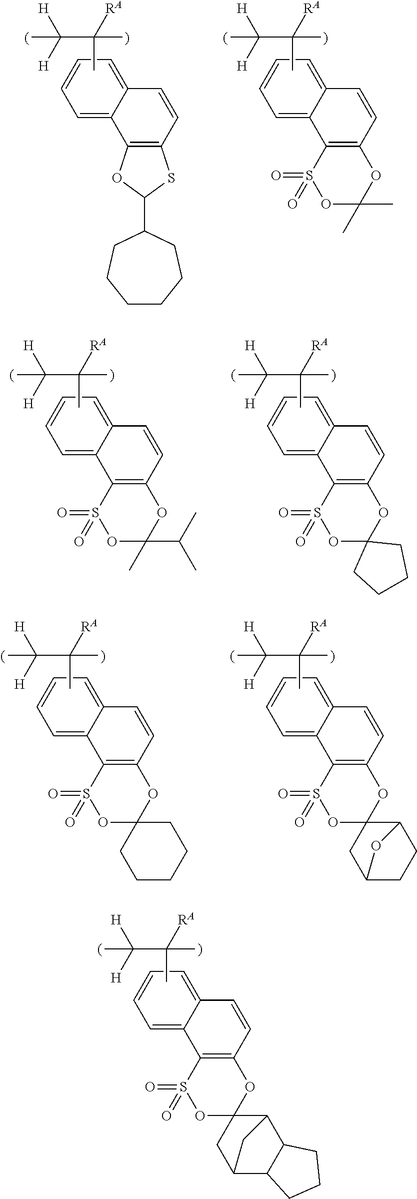

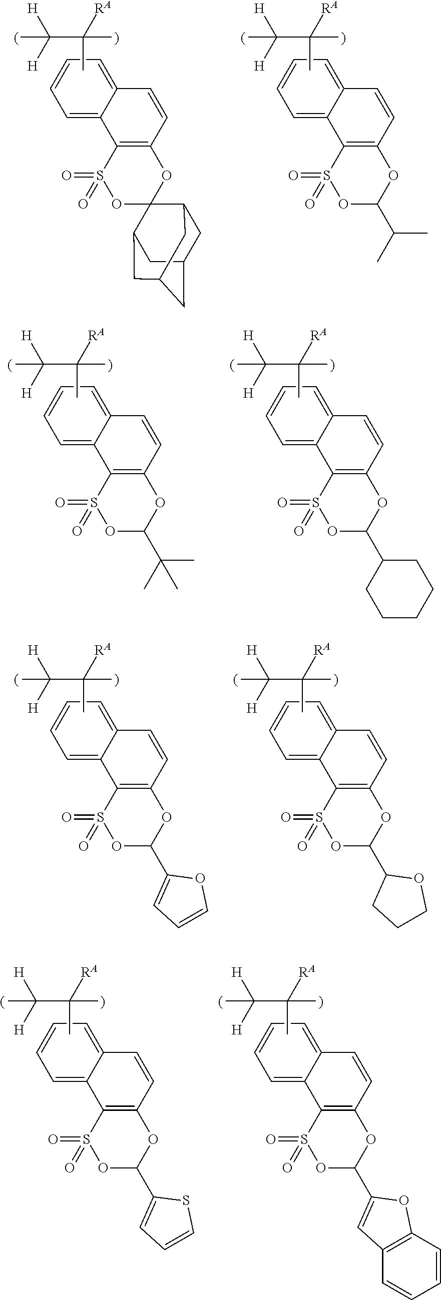

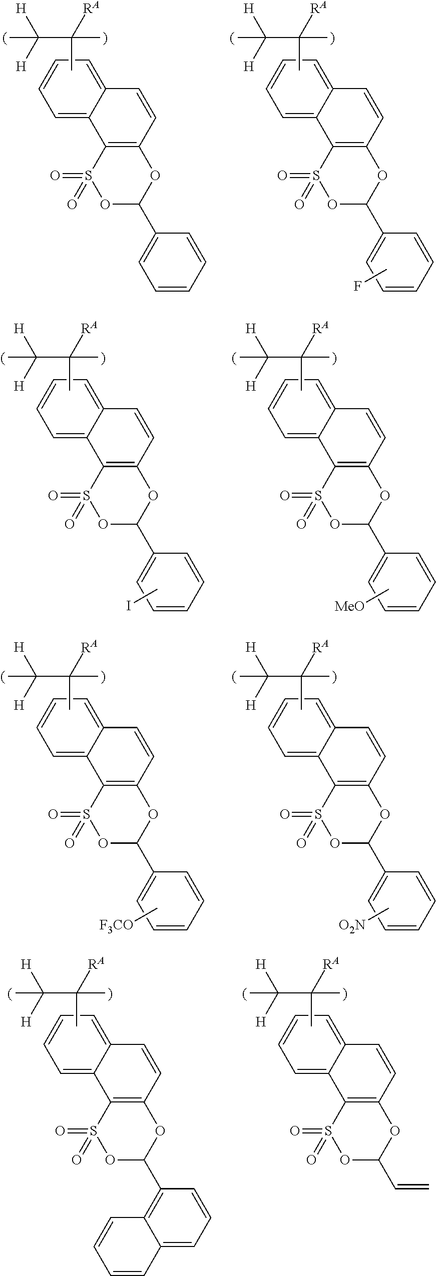

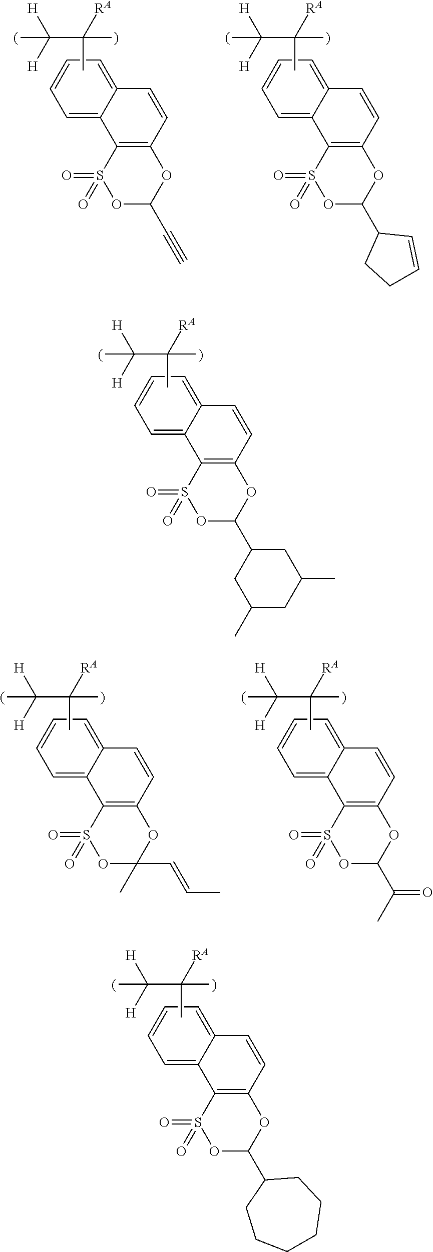

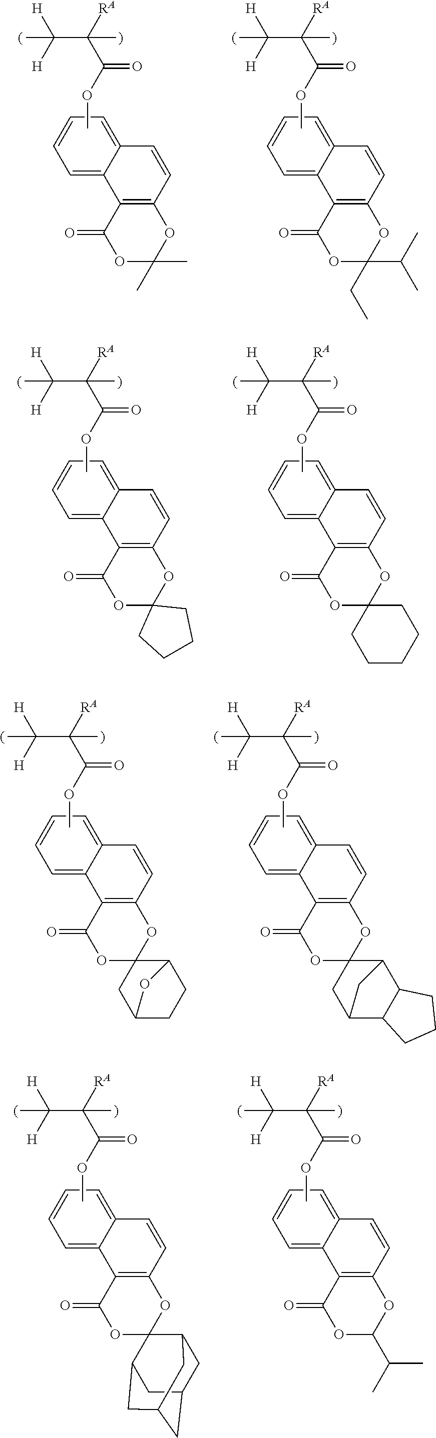

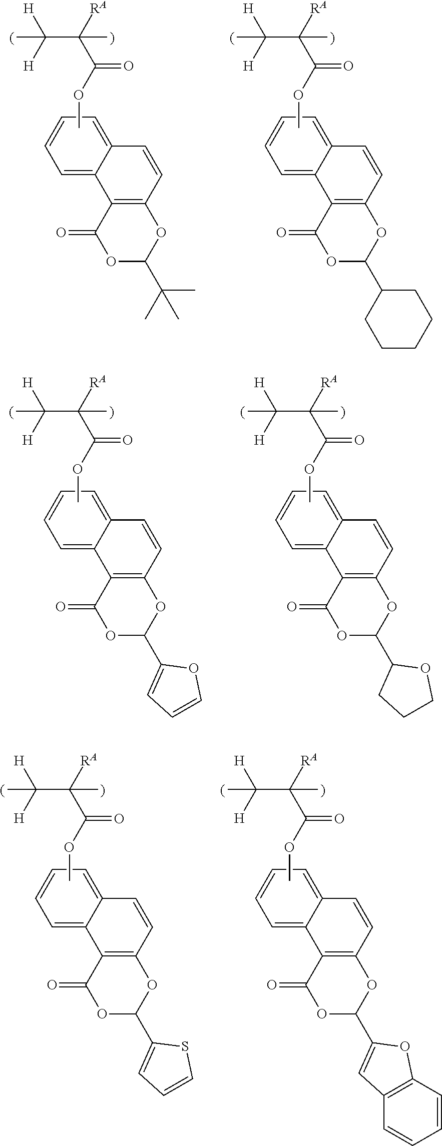

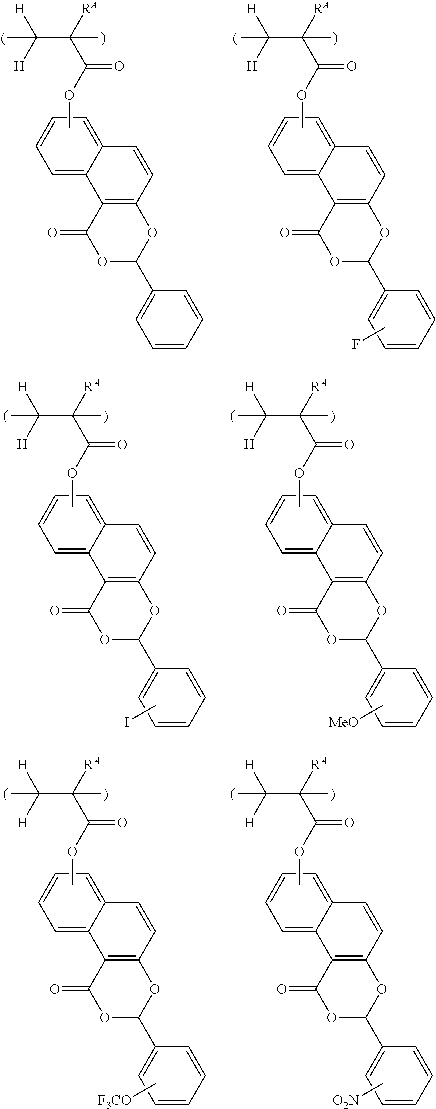

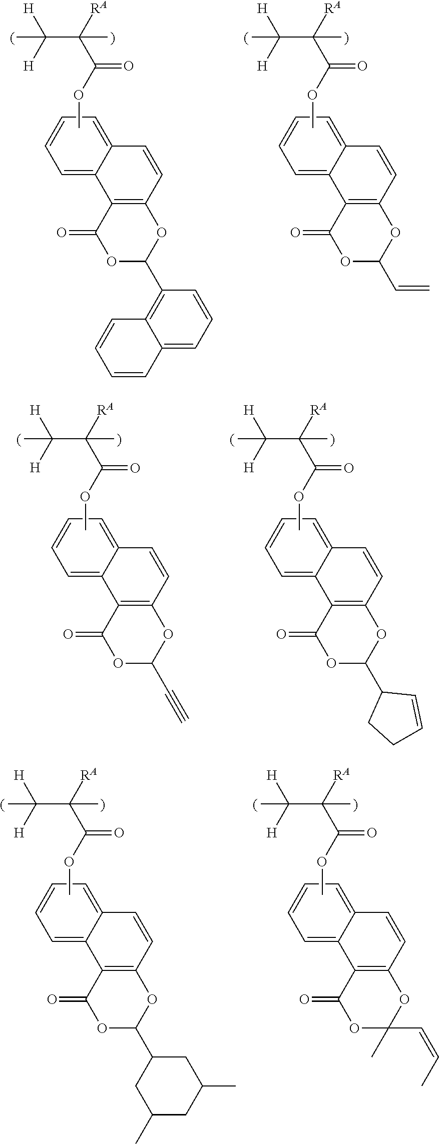

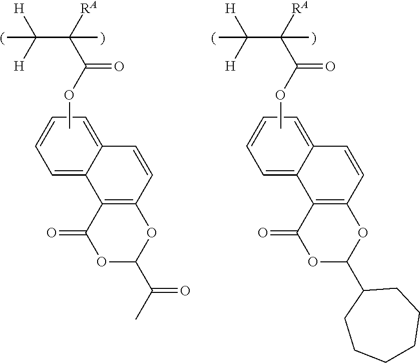

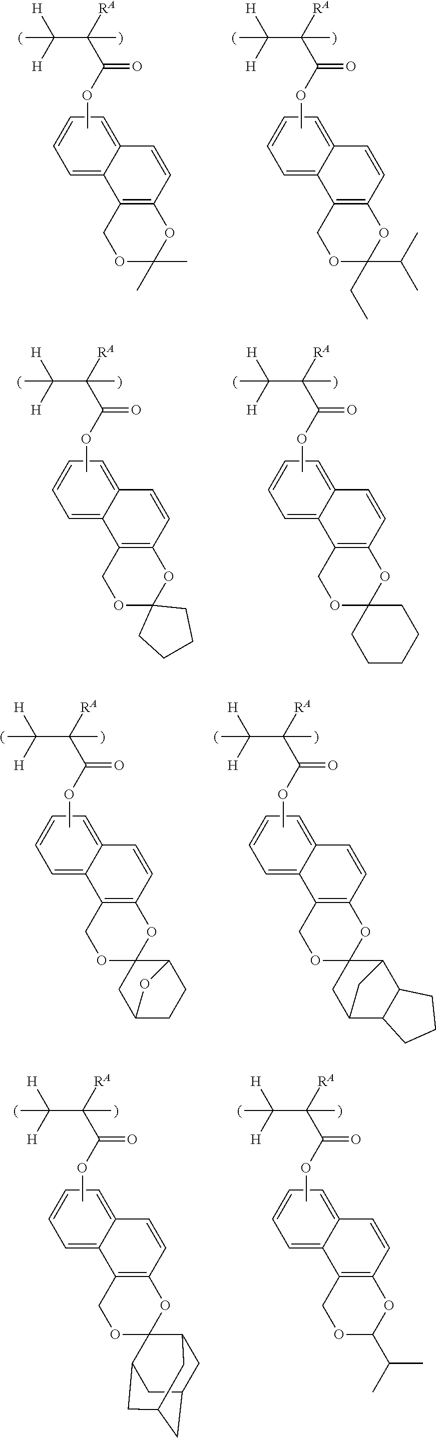

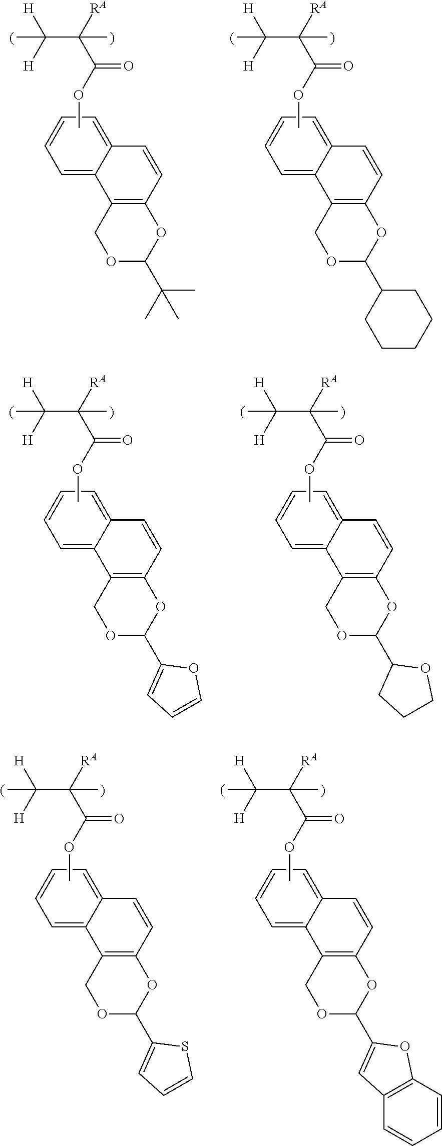

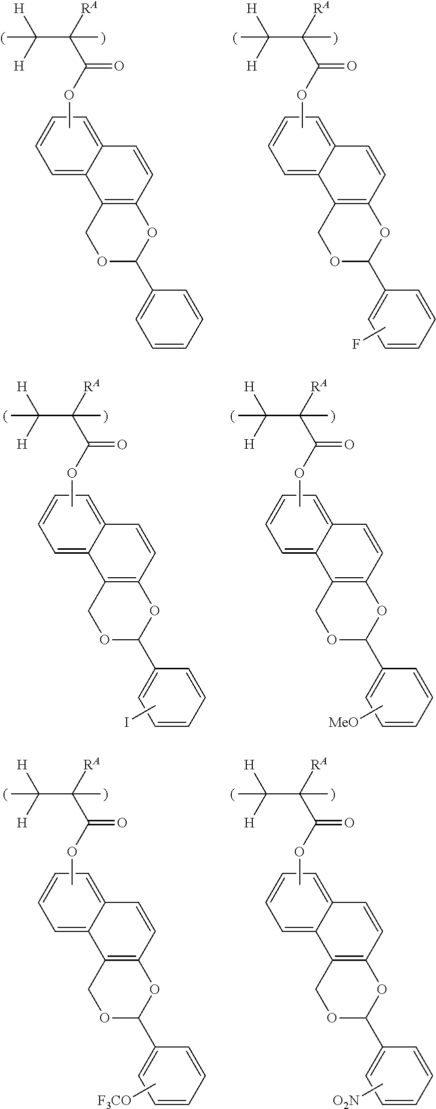

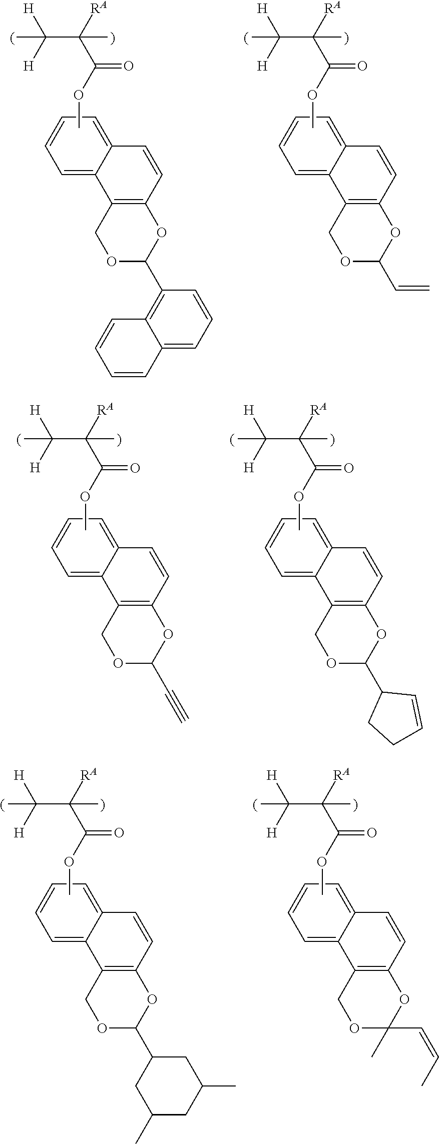

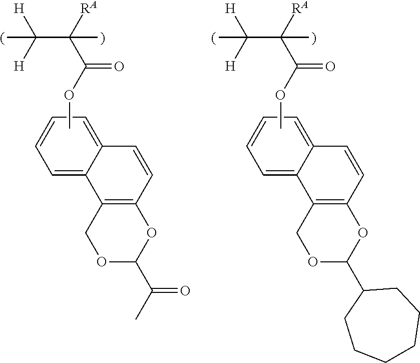

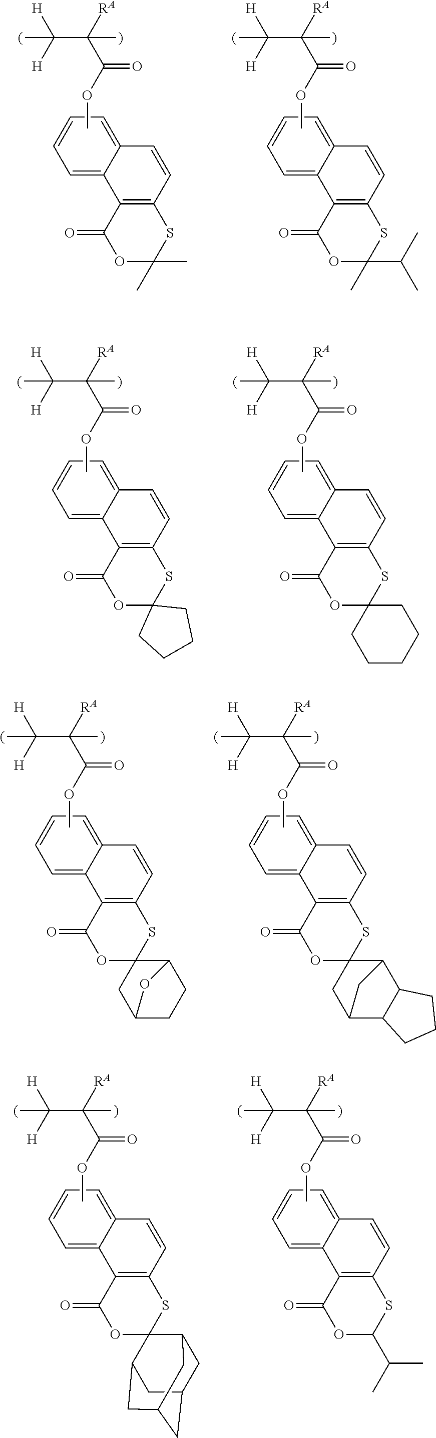

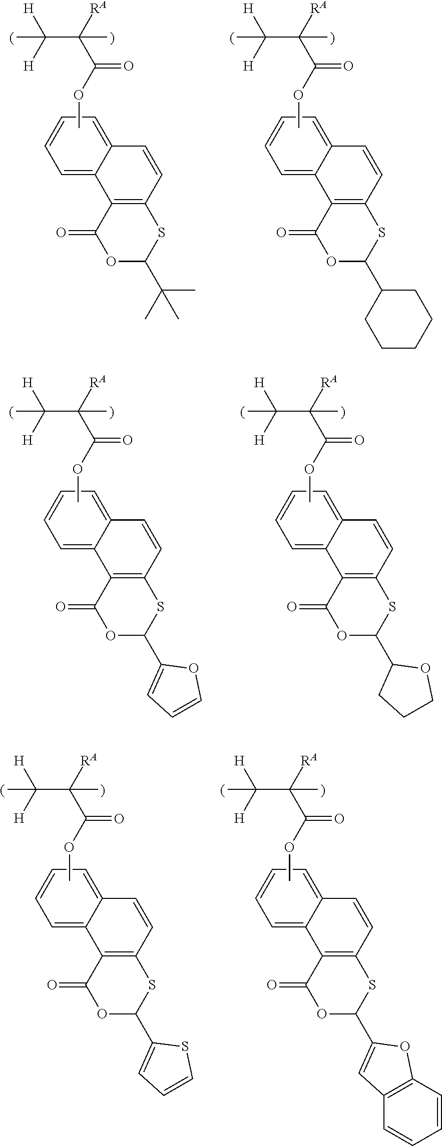

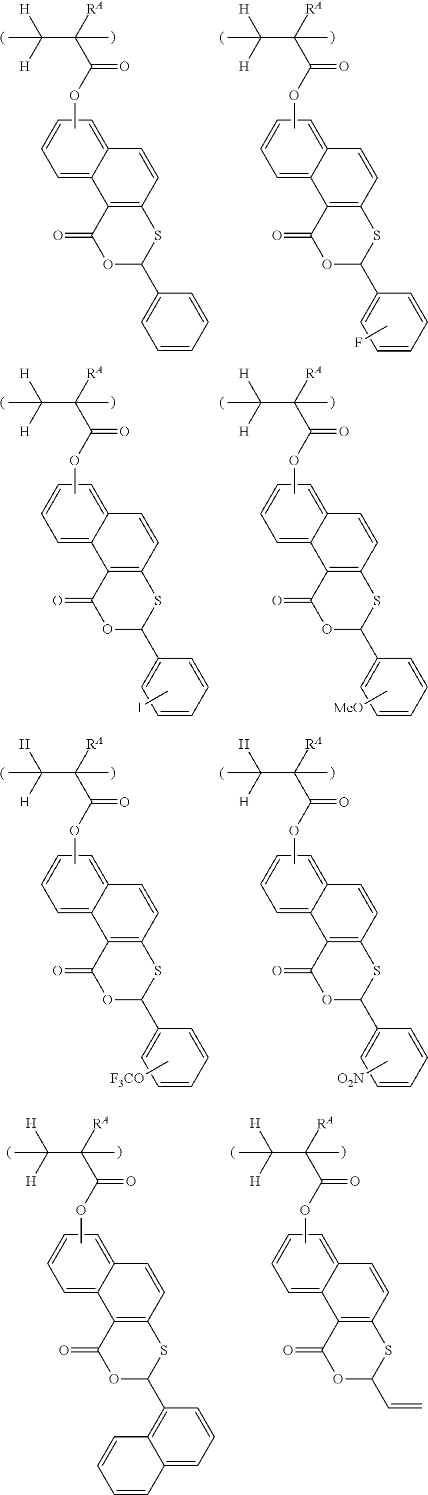

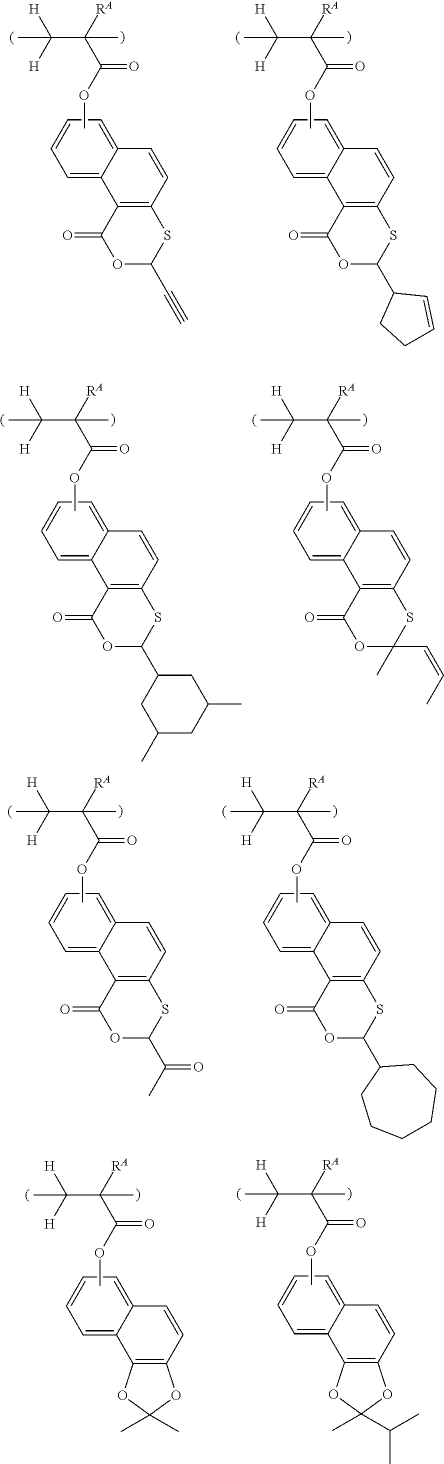

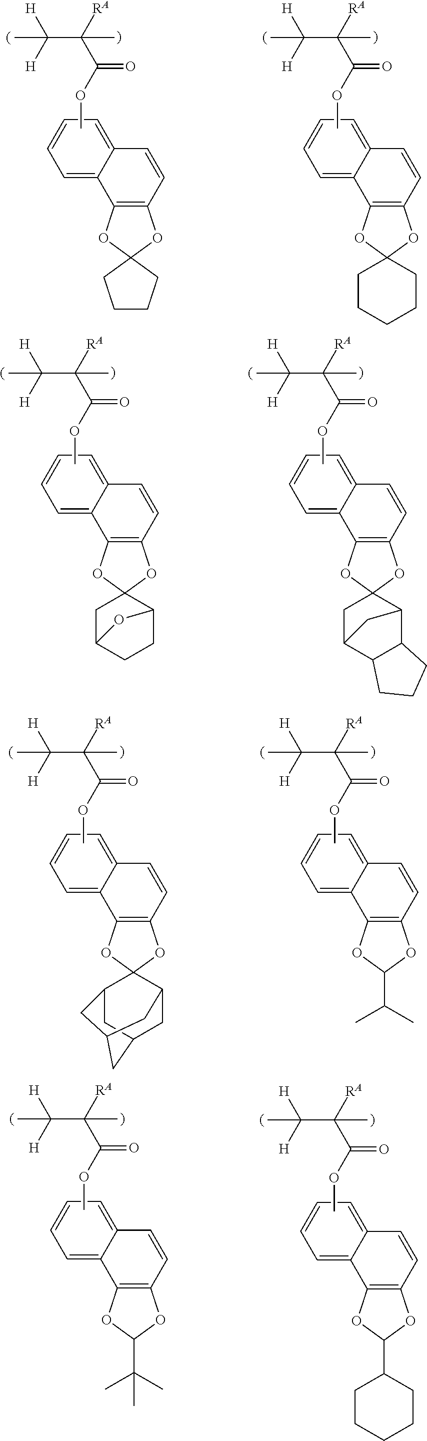

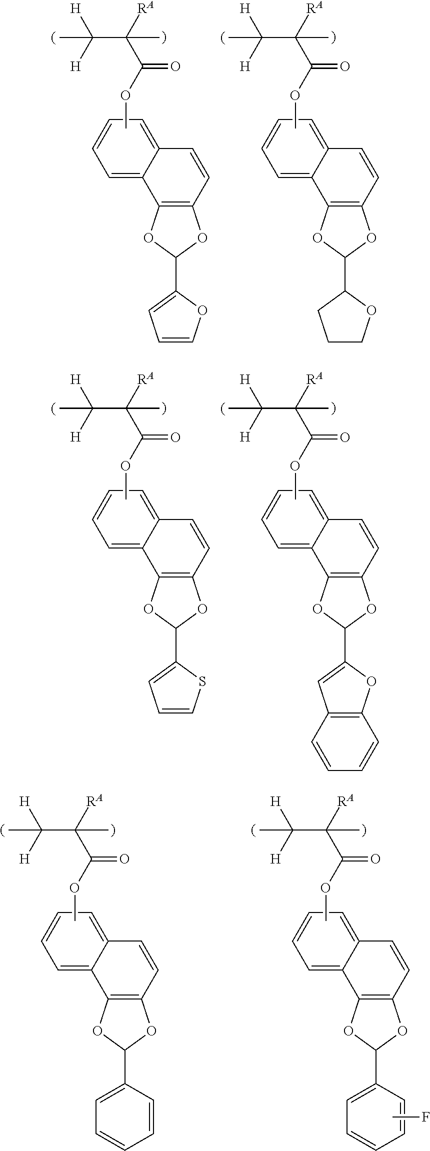

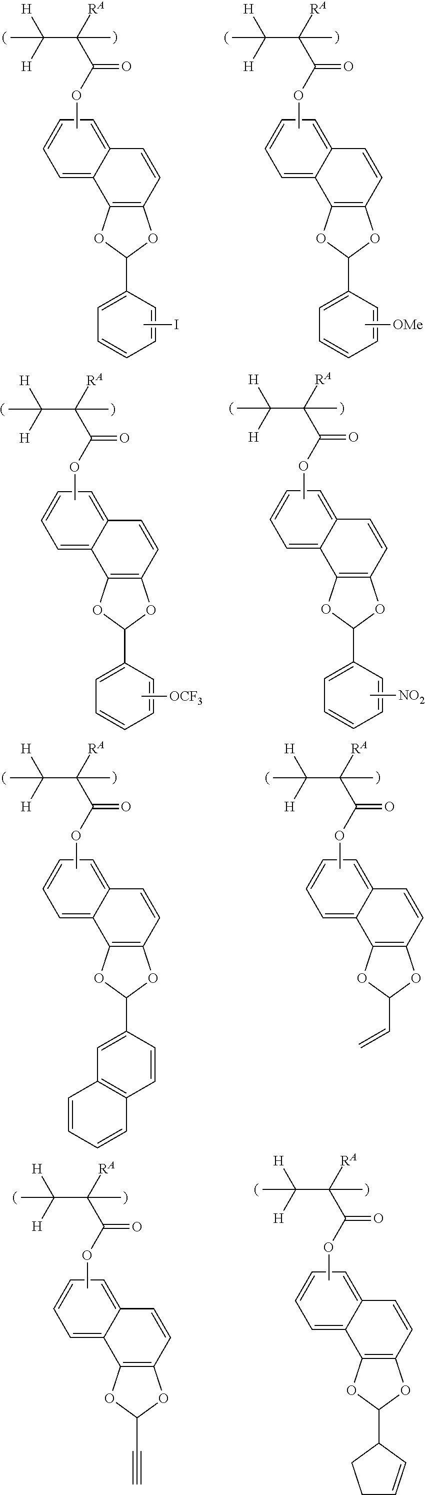

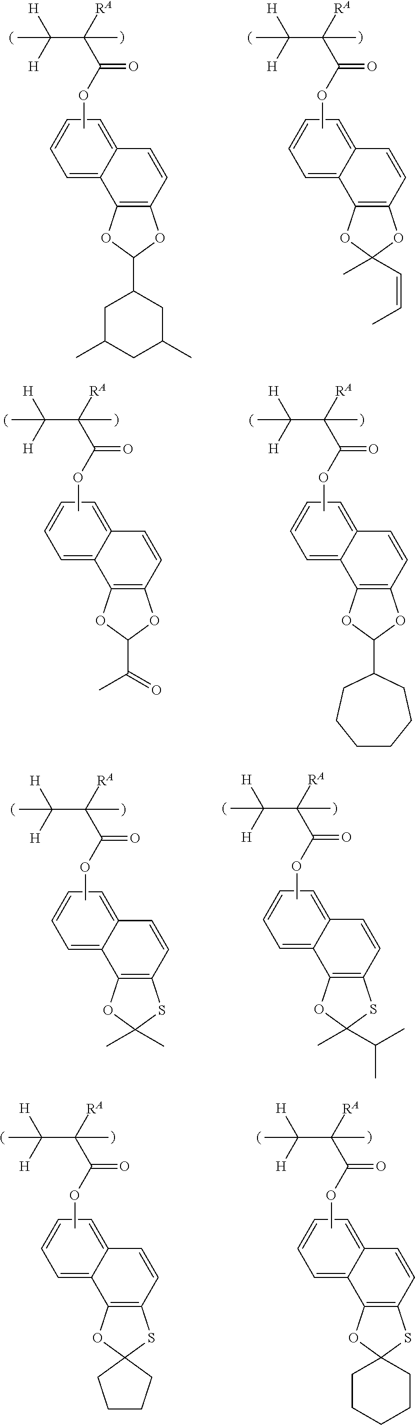

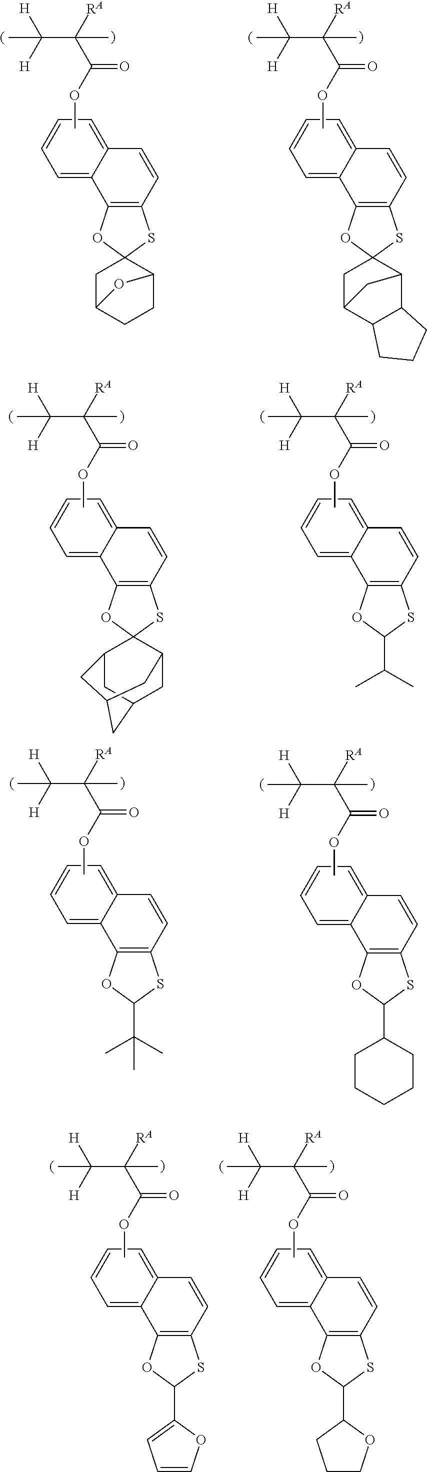

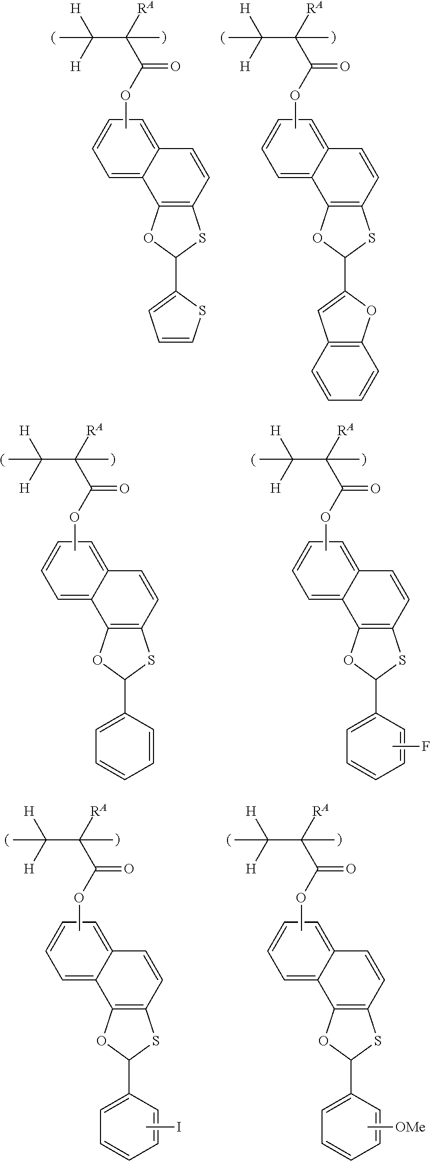

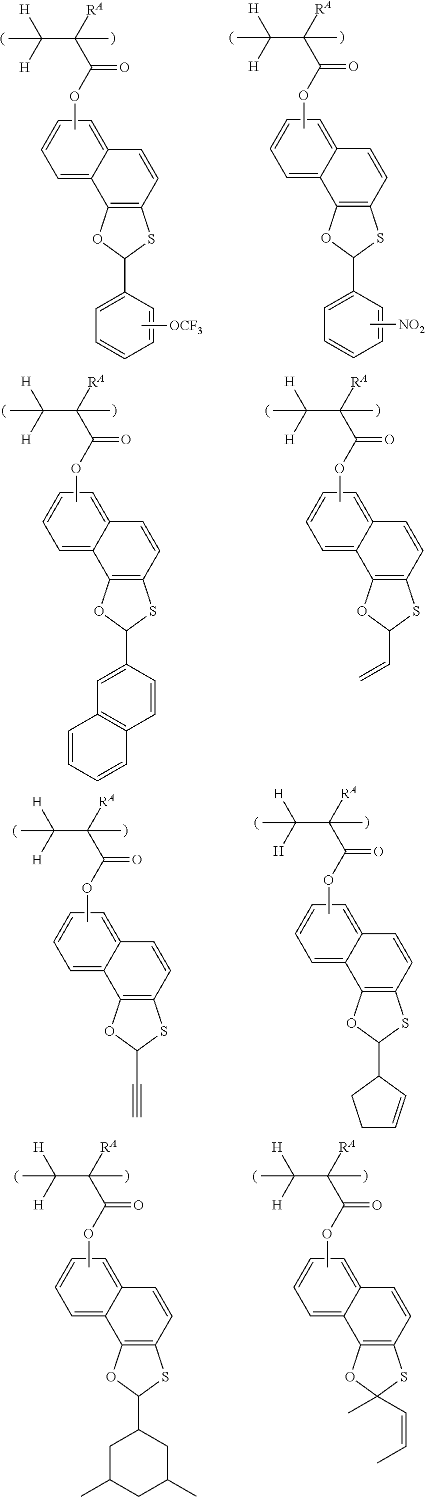

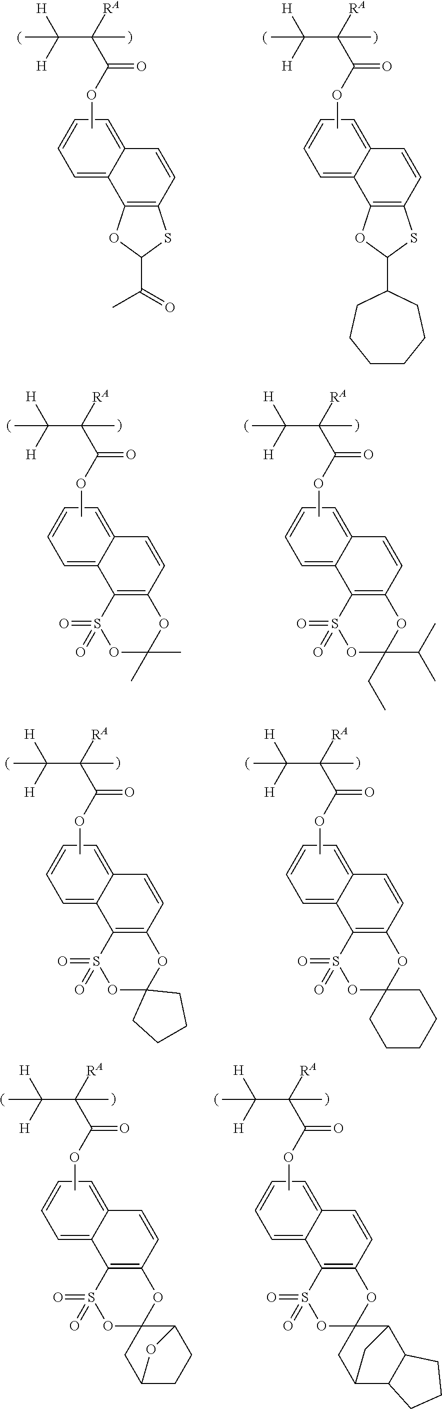

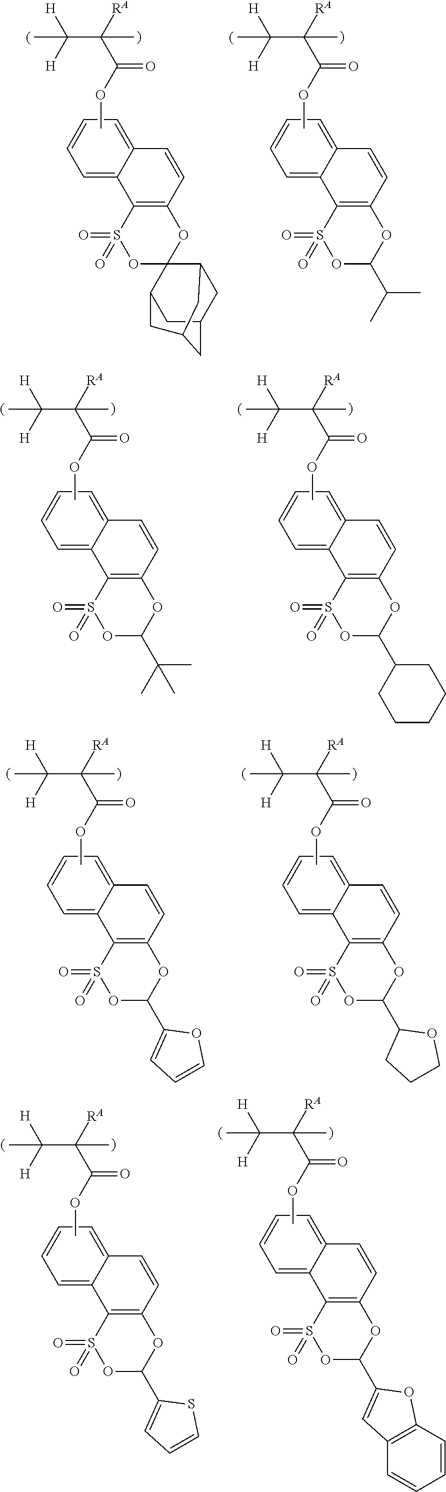

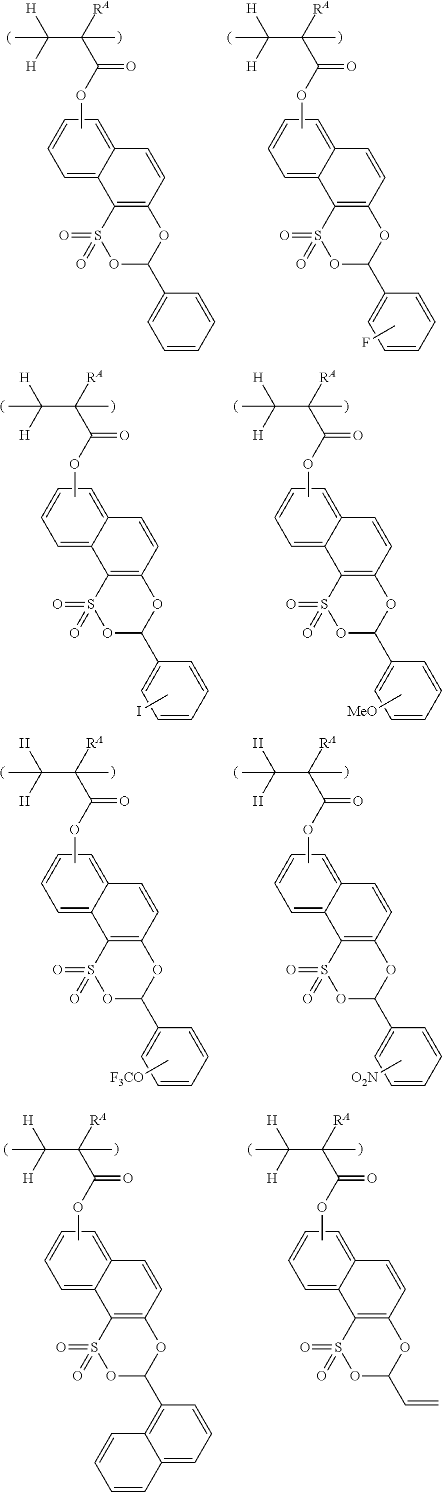

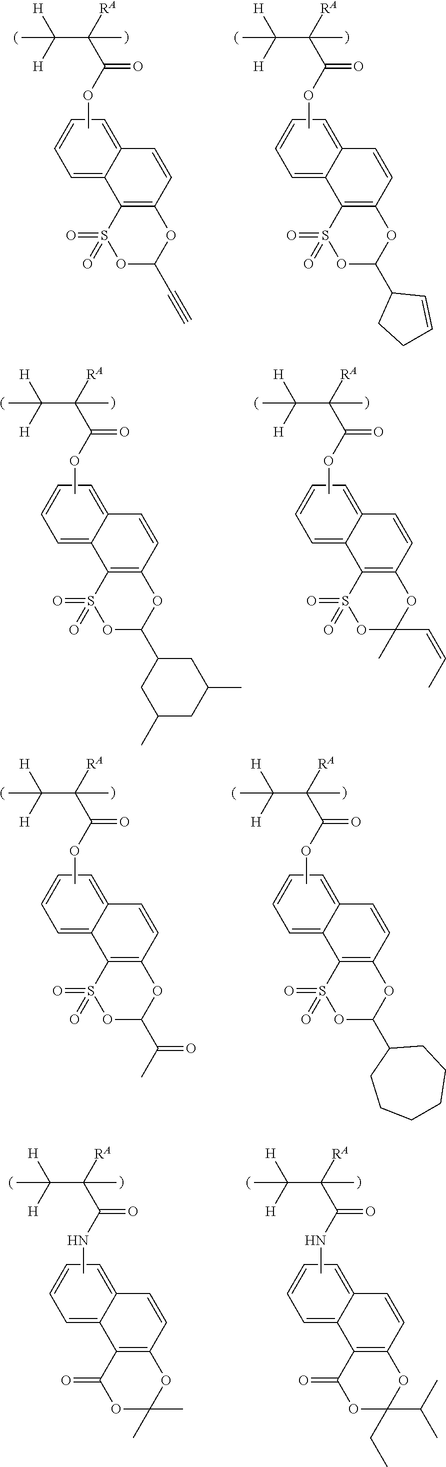

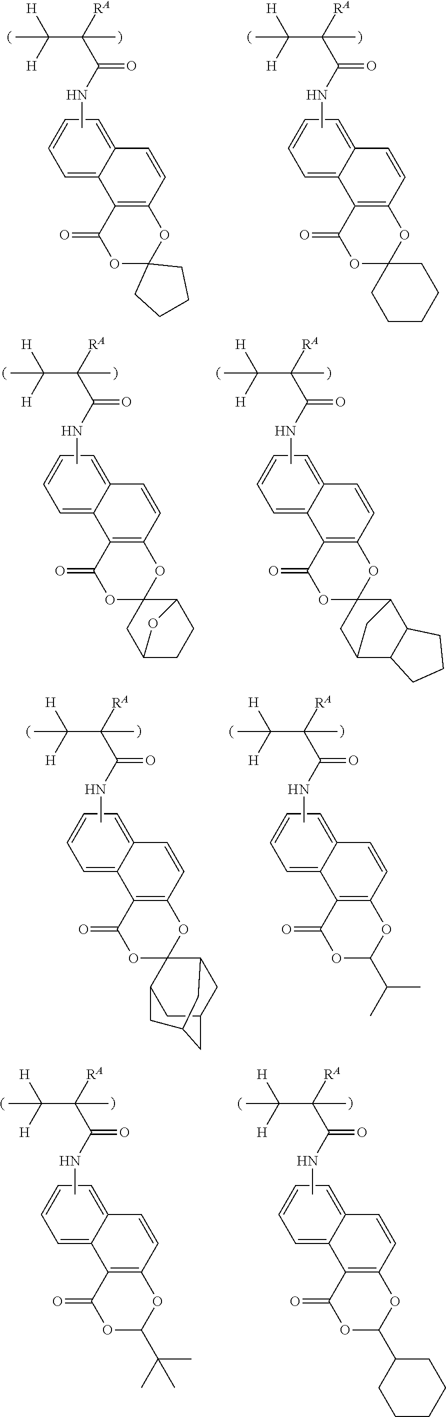

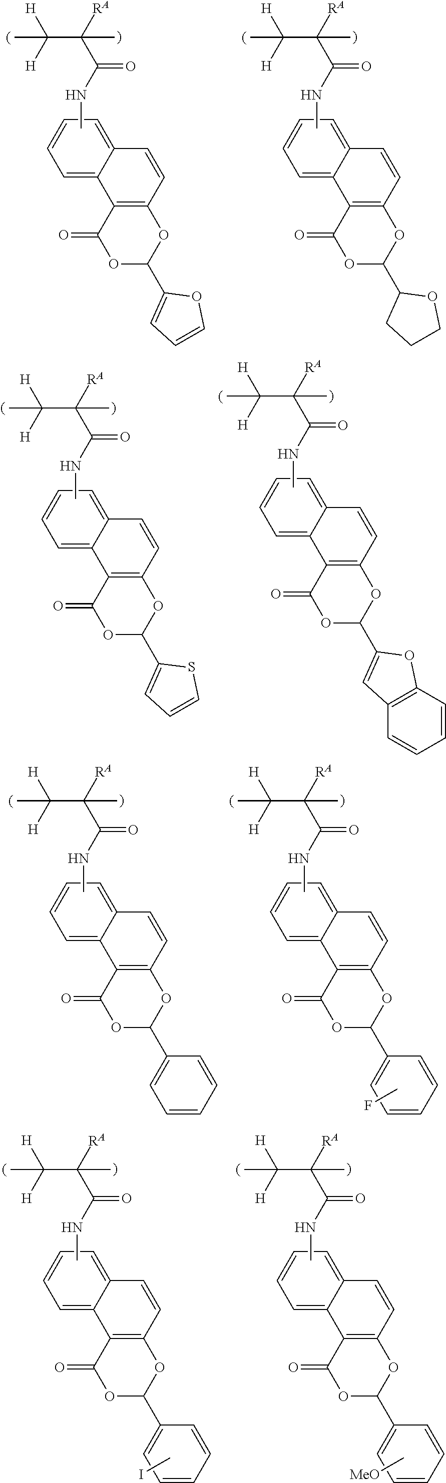

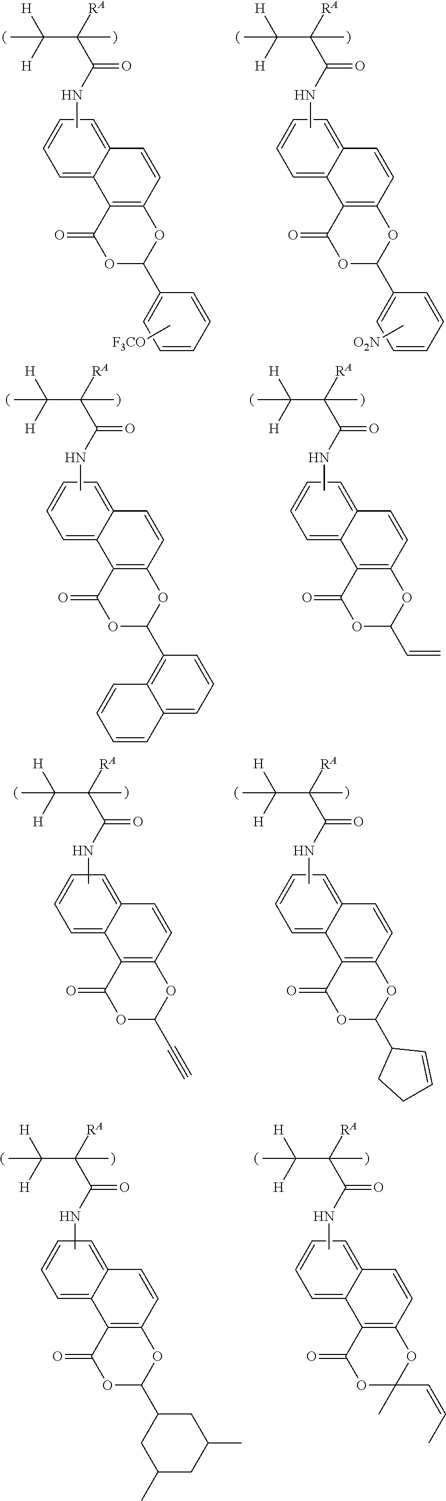

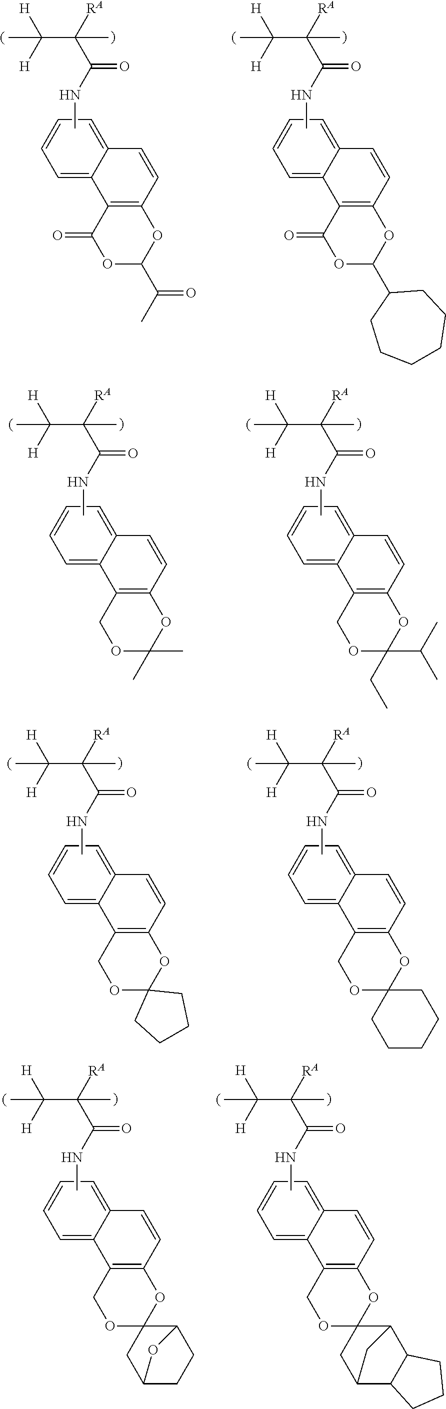

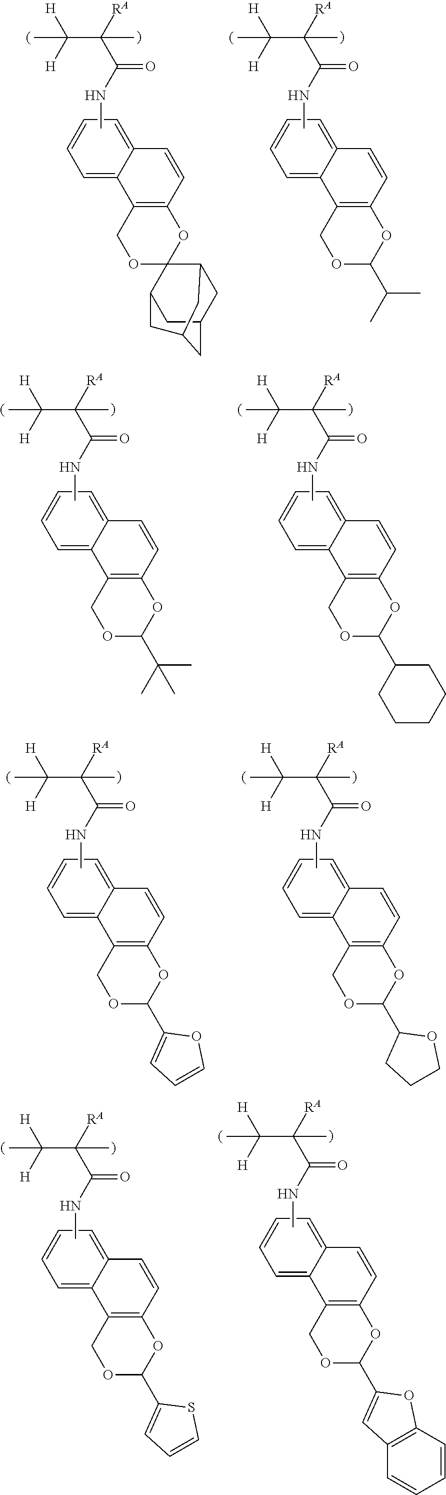

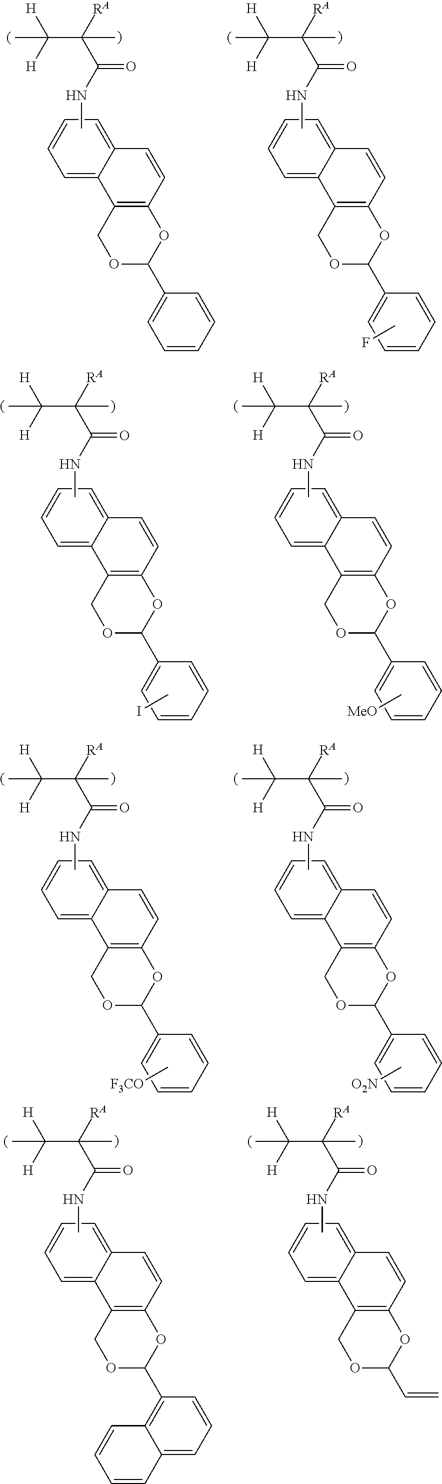

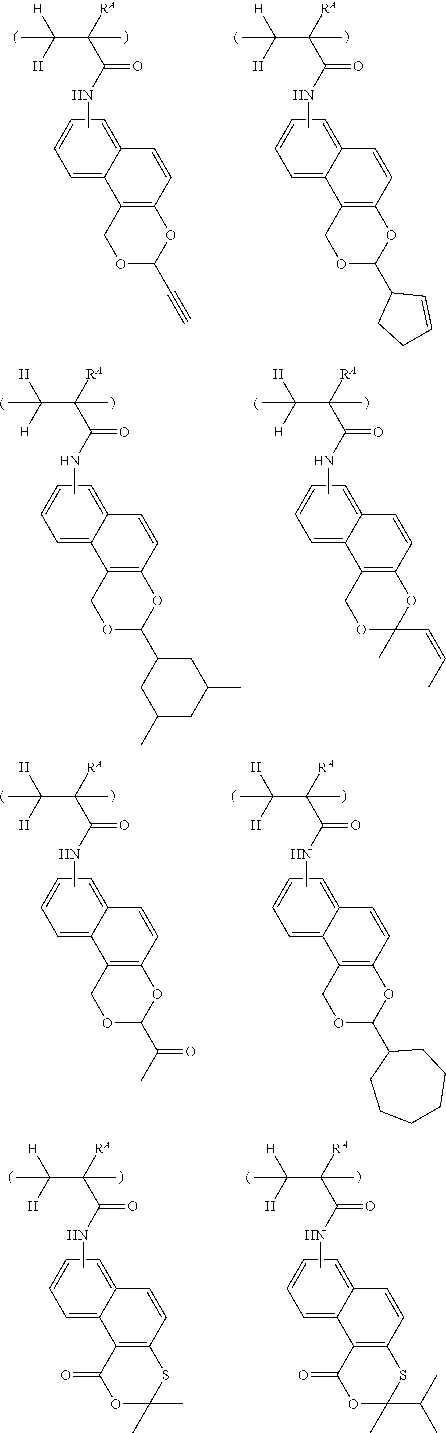

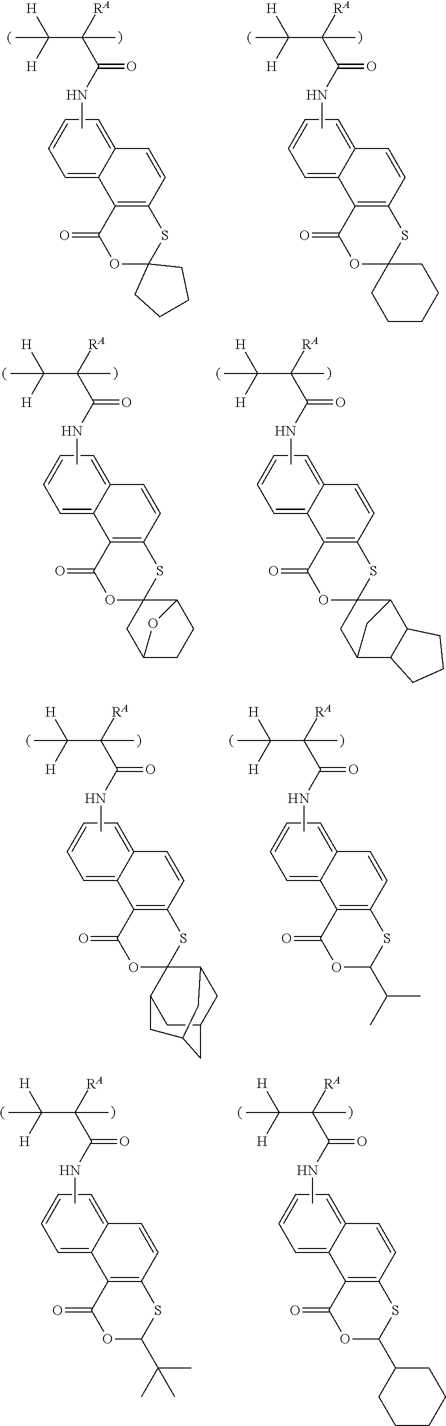

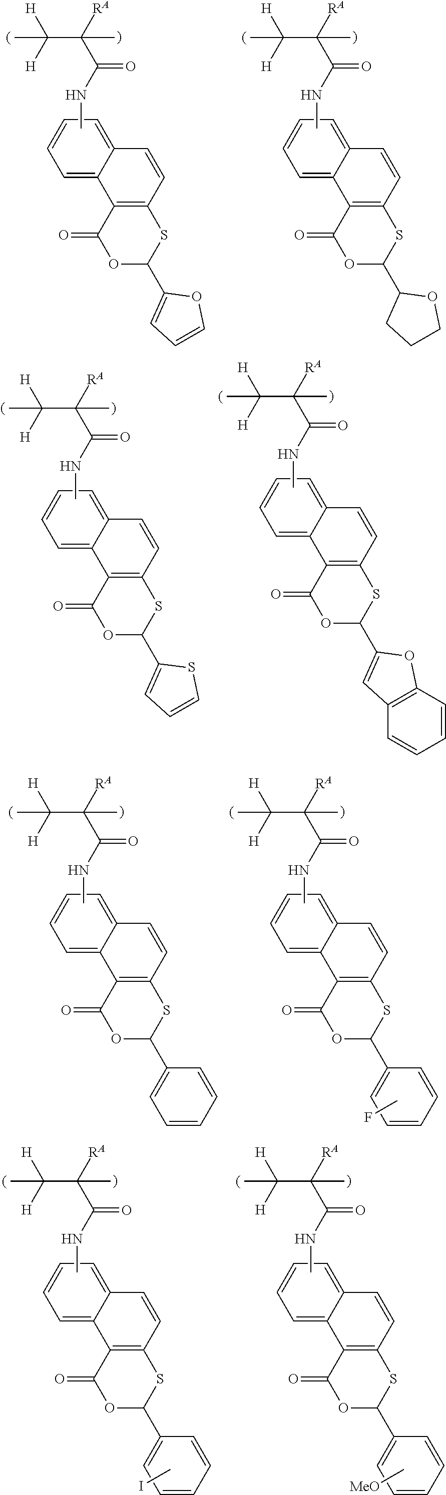

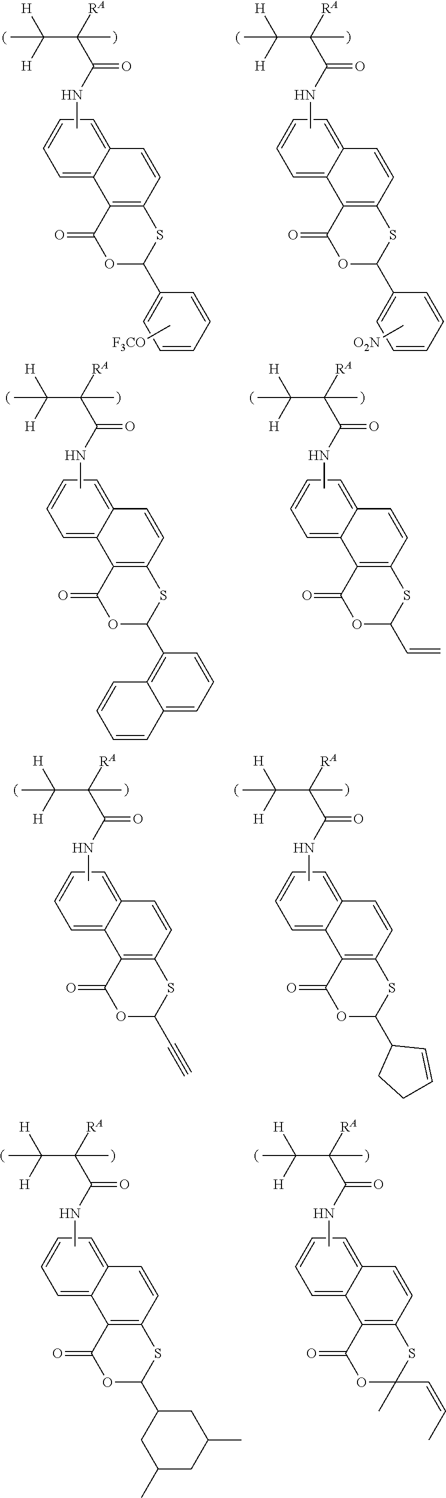

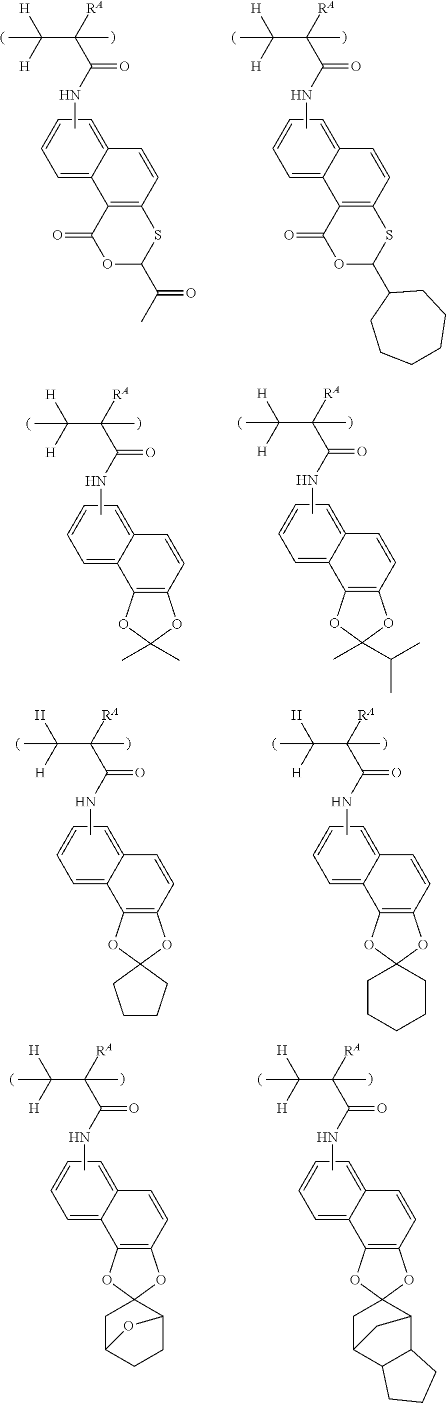

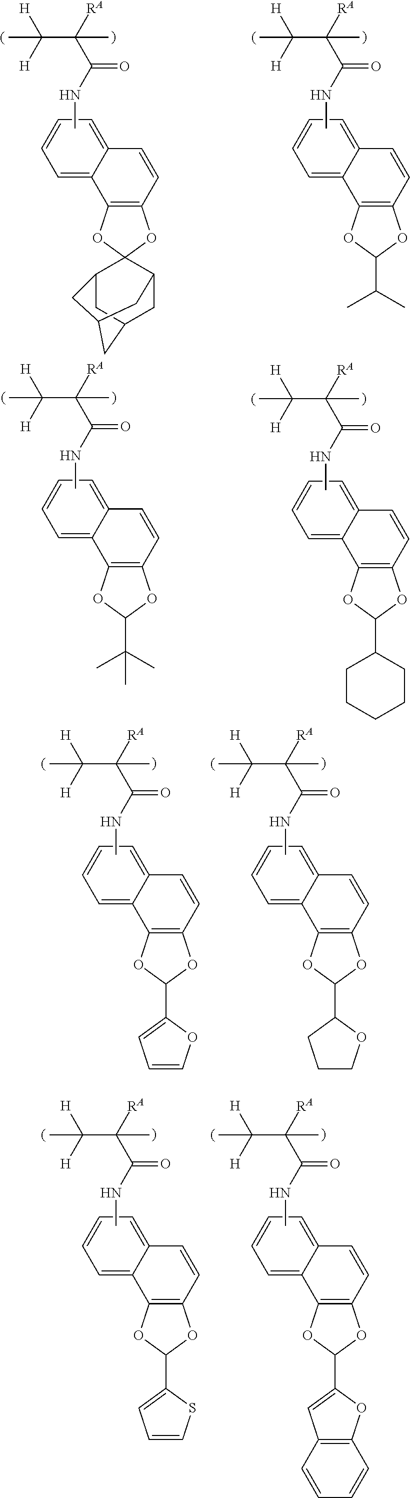

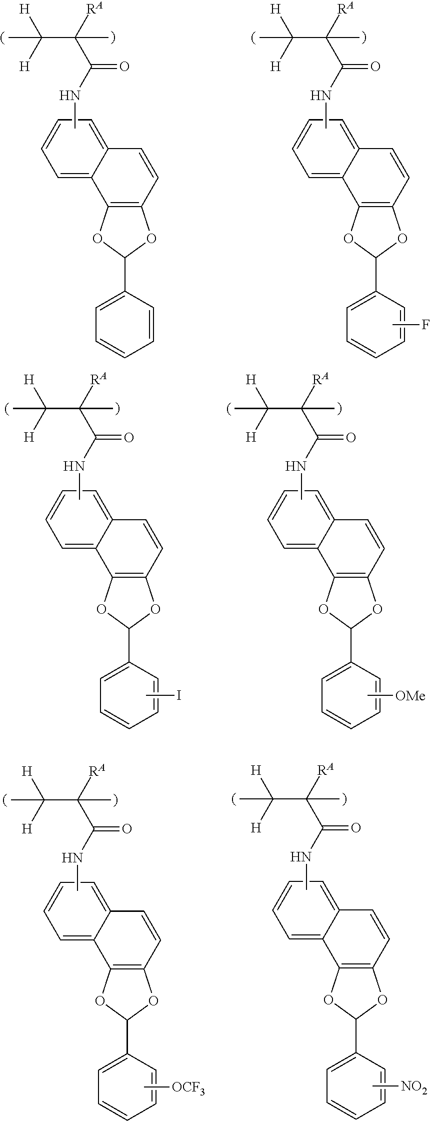

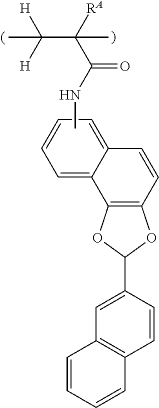

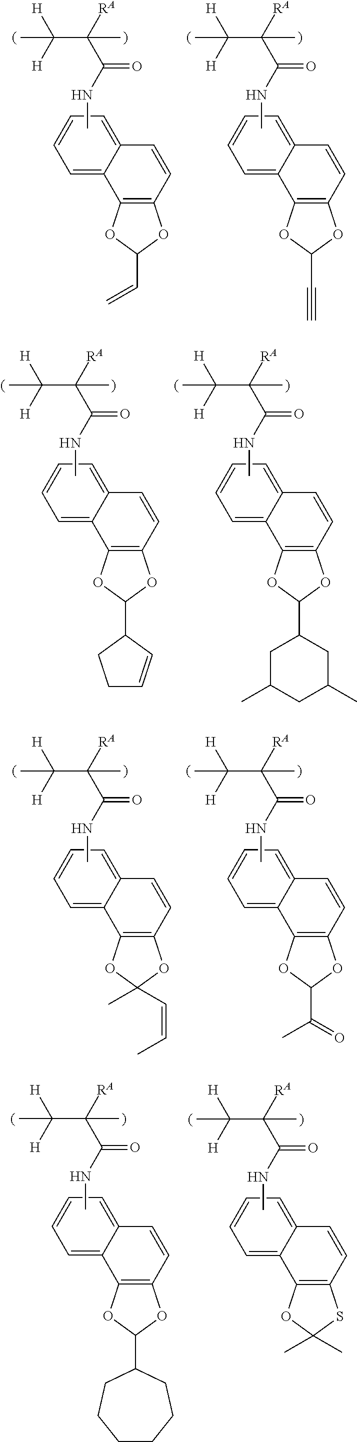

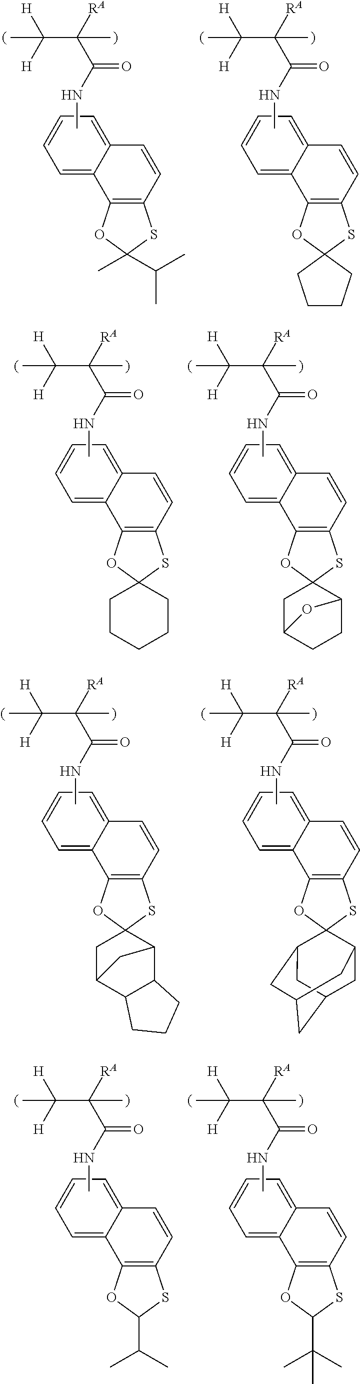

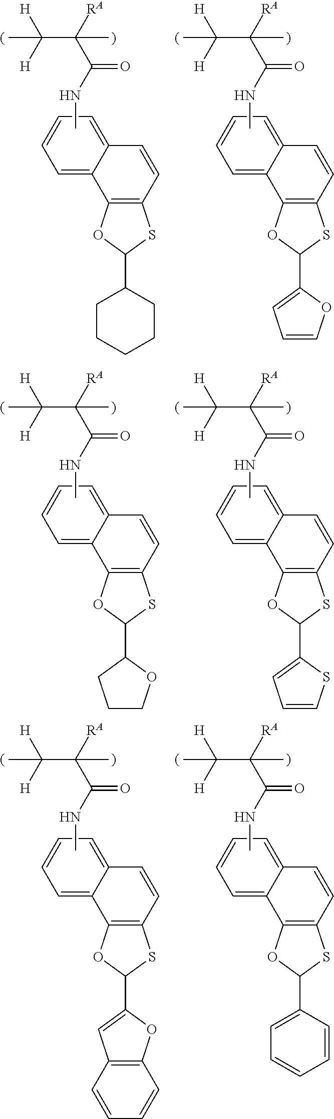

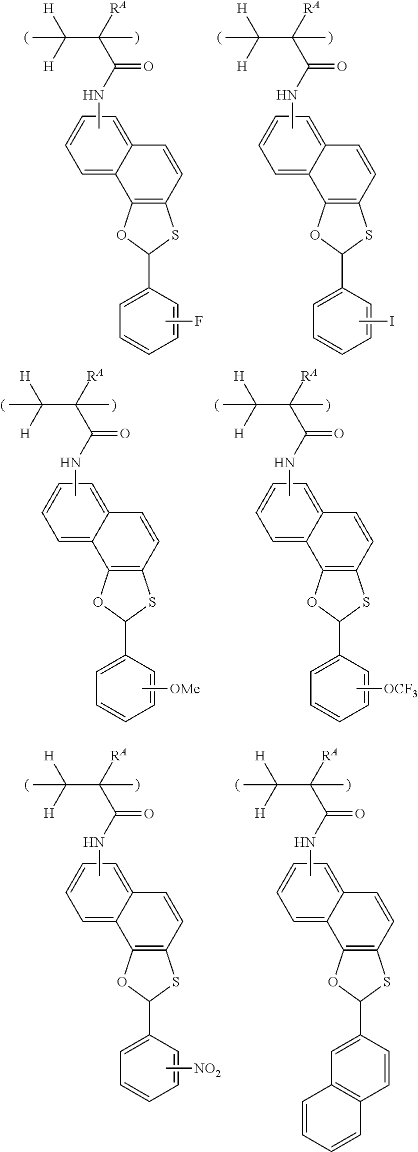

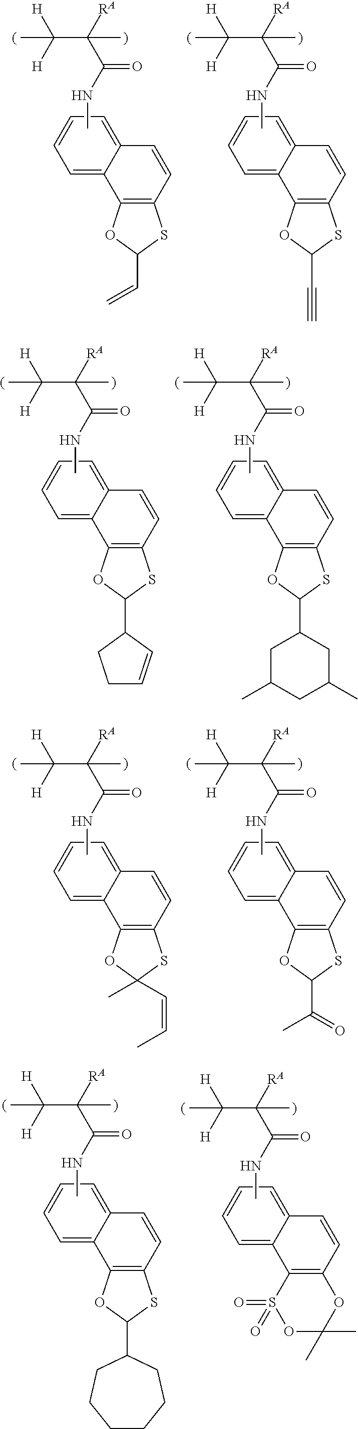

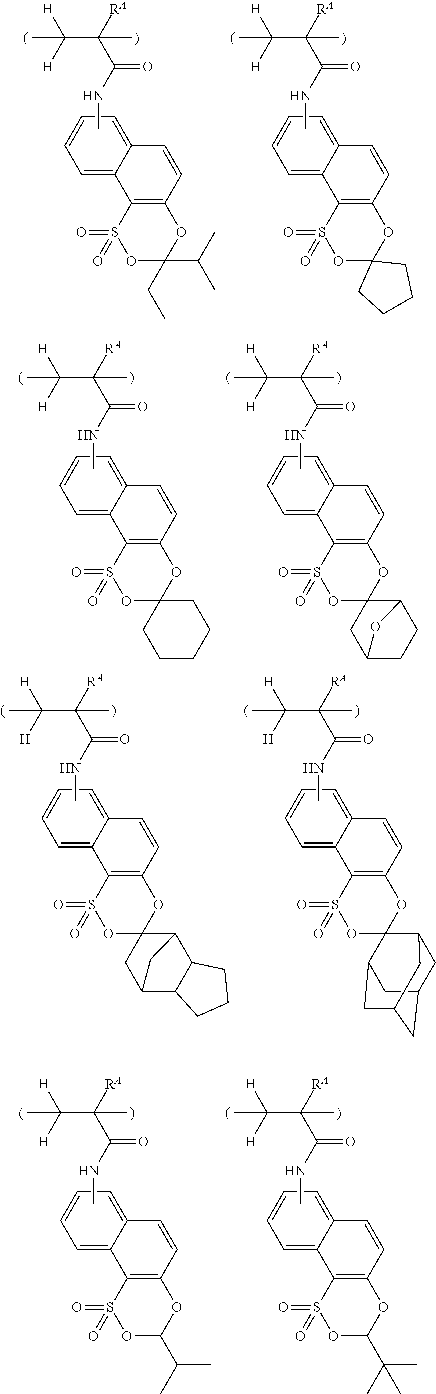

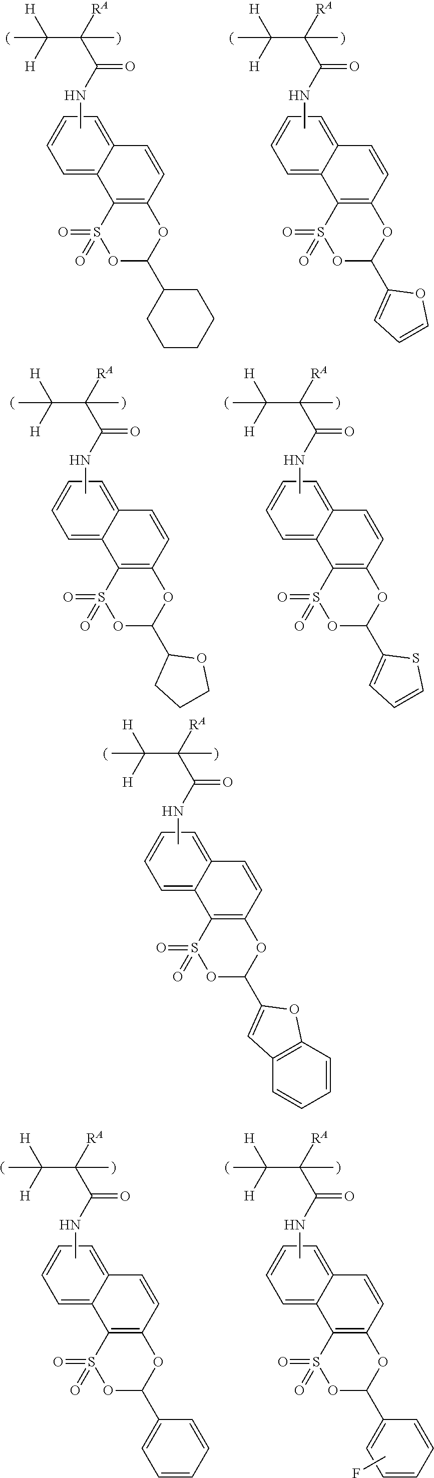

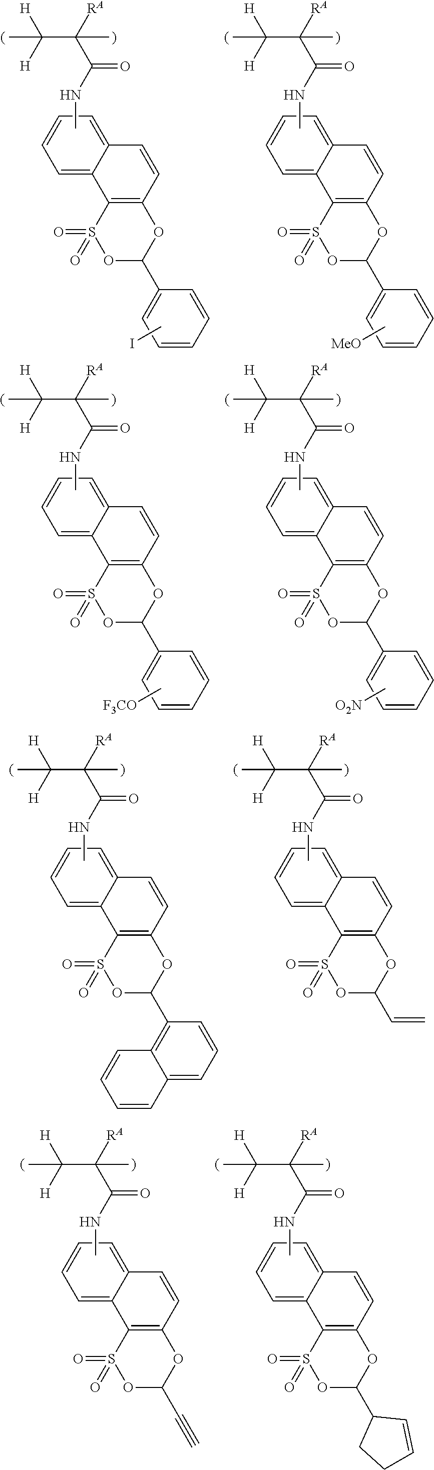

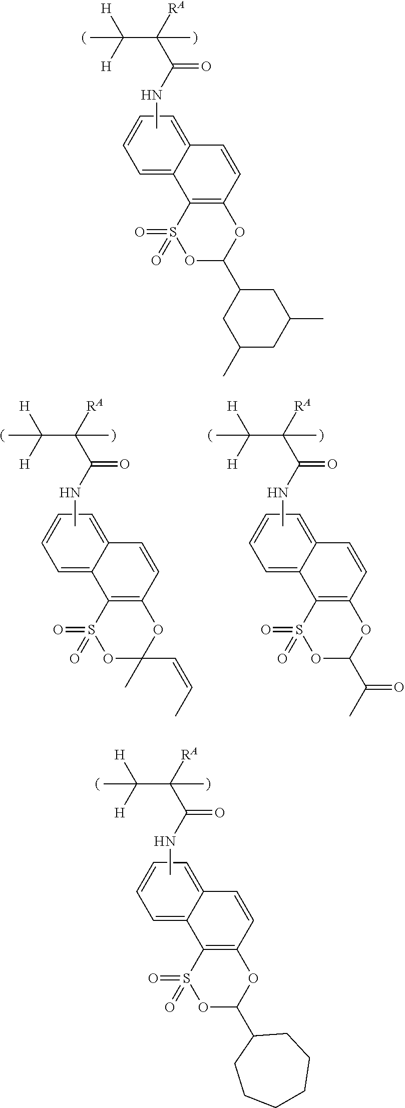

The base polymer preferably may comprise repeat units having the formula (b1), which are also referred to as repeat units (b1) or repeat units having the formula (b2), which are also referred to as repeat units (b2).

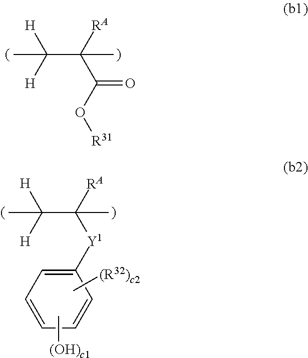

In the formulae (b1) and (b2), R^{A} is each independently hydrogen, fluorine, methyl or trifluoromethyl,
Y¹ is a single bond or *—C(═O)—O—, the asterisk (*) designates a point of attachment to the carbon atom in the backbone,
R³¹ is hydrogen or a C₁-C₂₀ group containing at least one structure selected from among hydroxy other than phenolic hydroxy, cyano, carbonyl, carboxy, ether bond, ester bond, sulfonate ester bond, carbonate bond, lactone ring, sultone ring and carboxylic anhydride (—C(═O)—O—C(═O)—),
R³² is halogen, carboxy, nitro, cyano, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R³² may be identical or different when c2 is 2, 3, or 4, and
c1 is 1, 2, 3, or 4 and c2 is 0, 1, 2, 3, or 4, meeting 1≤c1+c2≤5.

Examples of the repeat units (b1) include those shown below, but not limited thereto. In the formulae. R^{A} is as defined above.

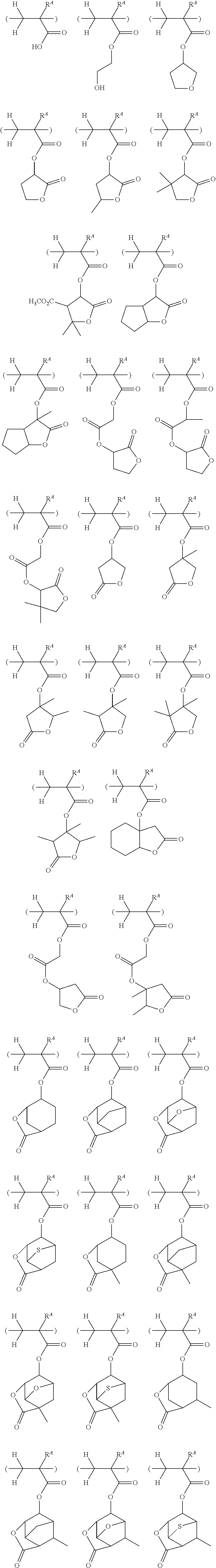

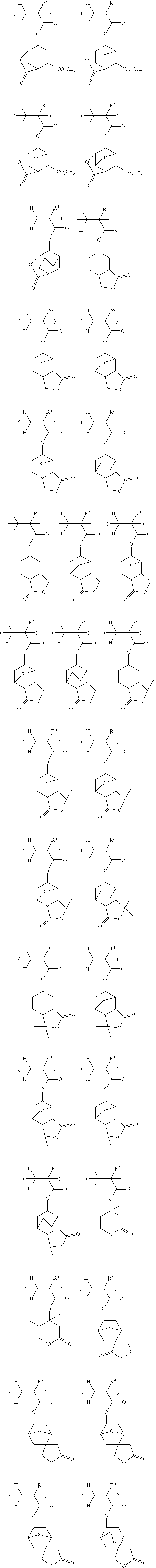

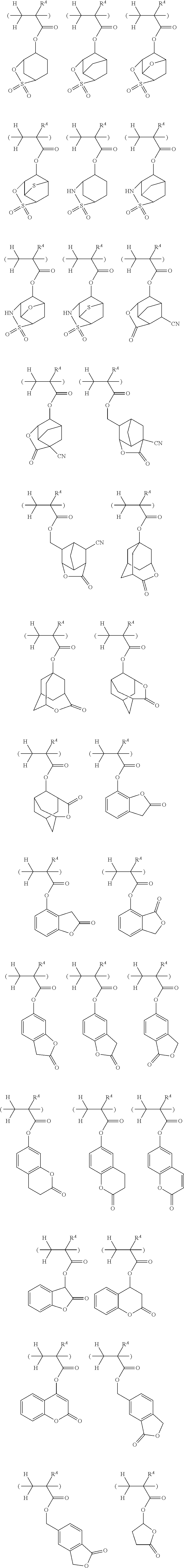

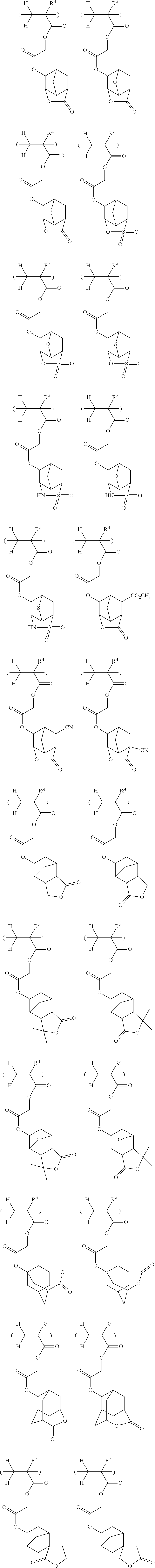

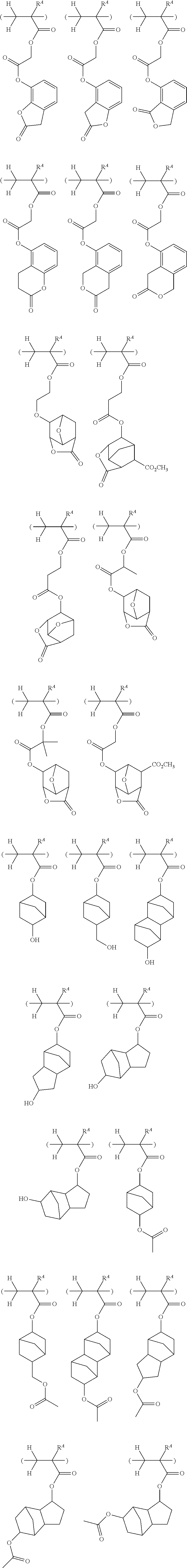

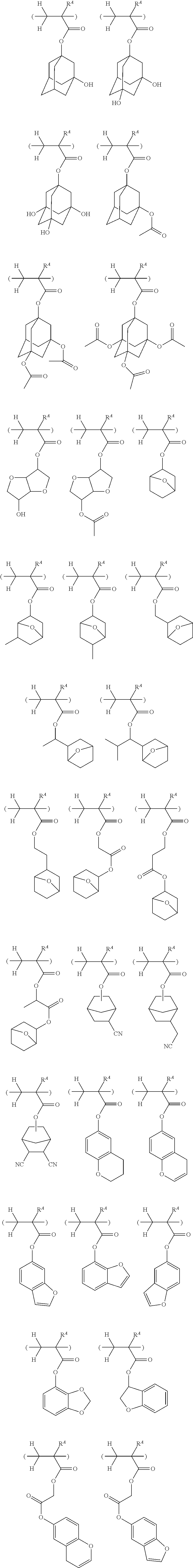

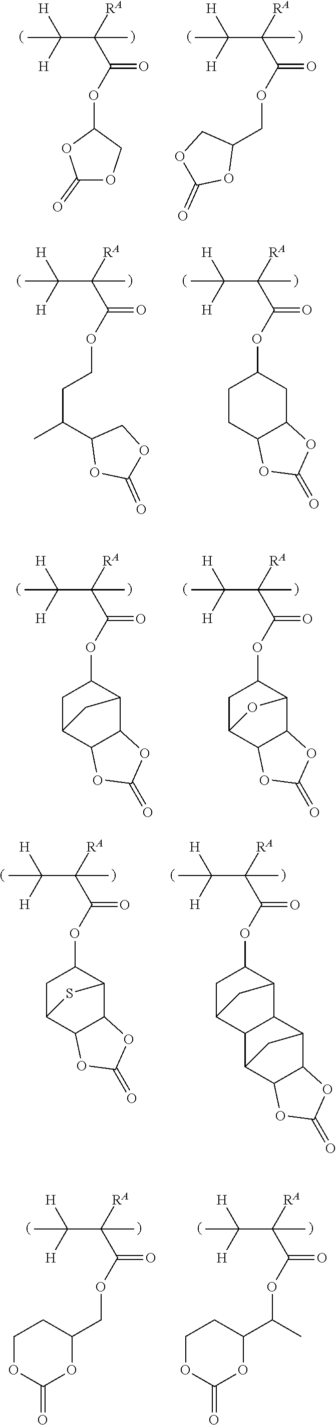

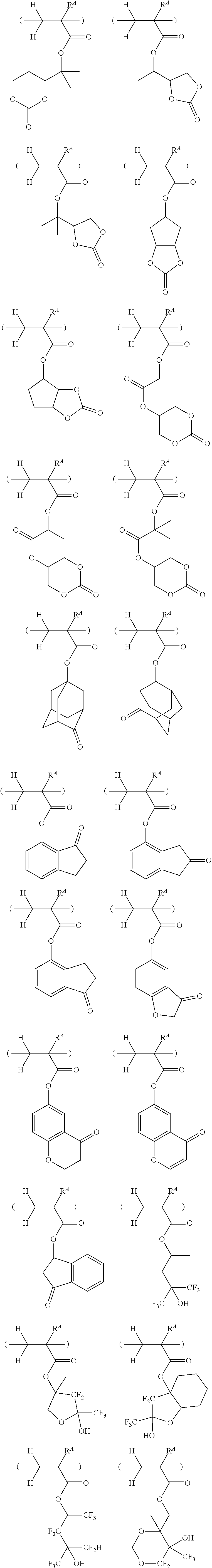

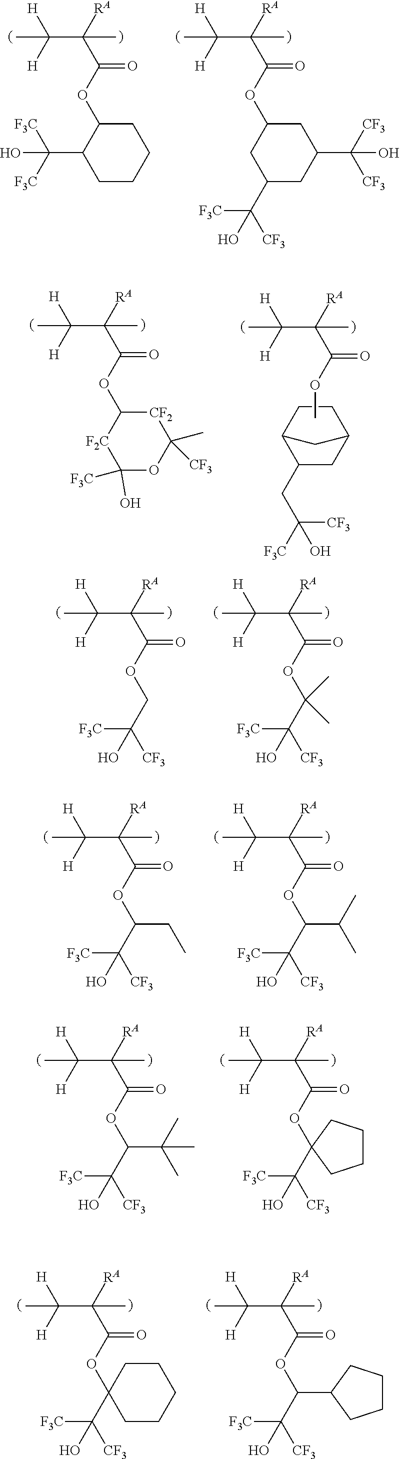

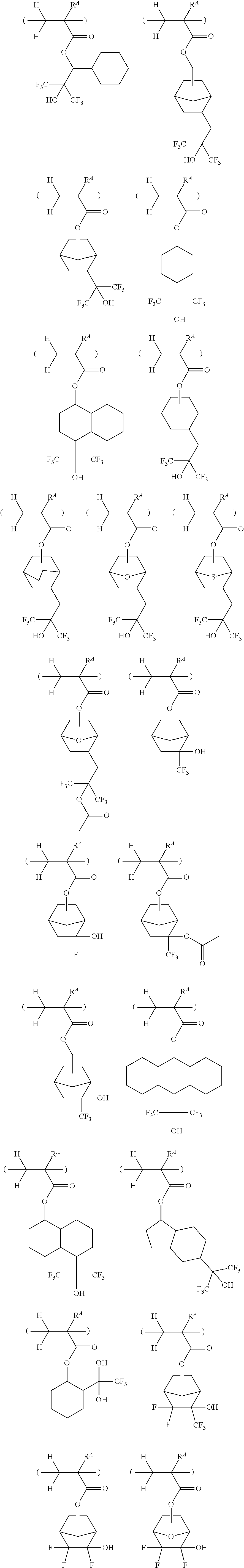

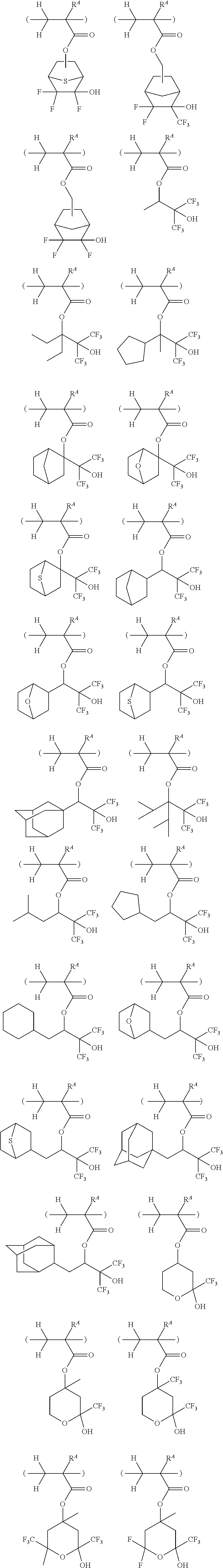

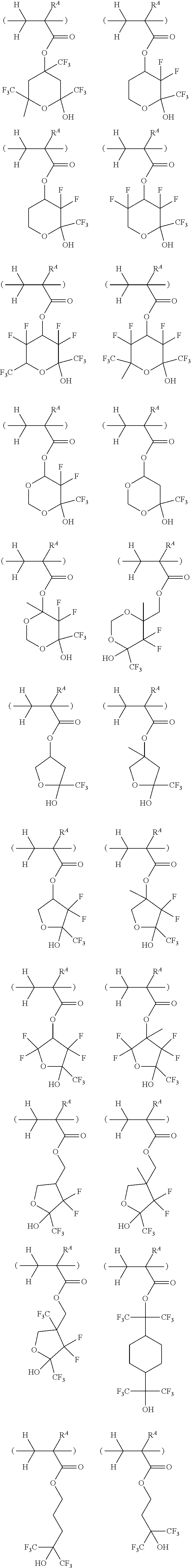

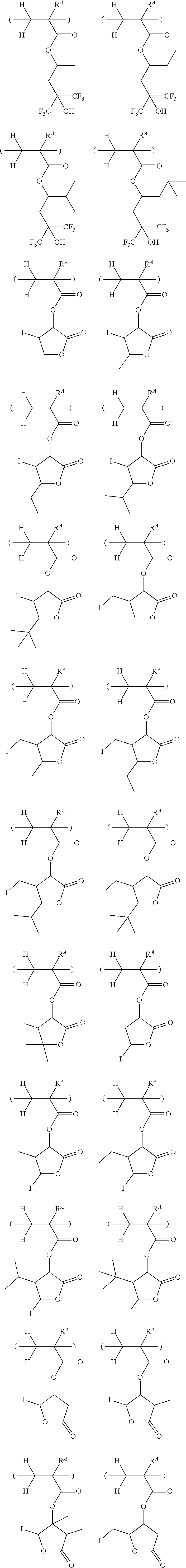

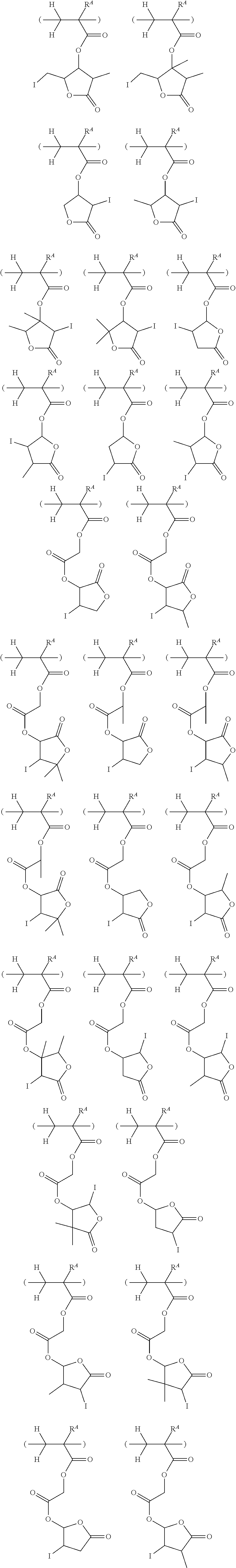

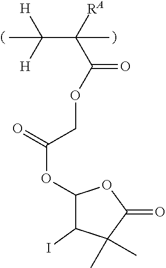

Examples of the repeat units (b2) include those shown below, but not limited thereto. In the formulae. R^{A} is as defined above.

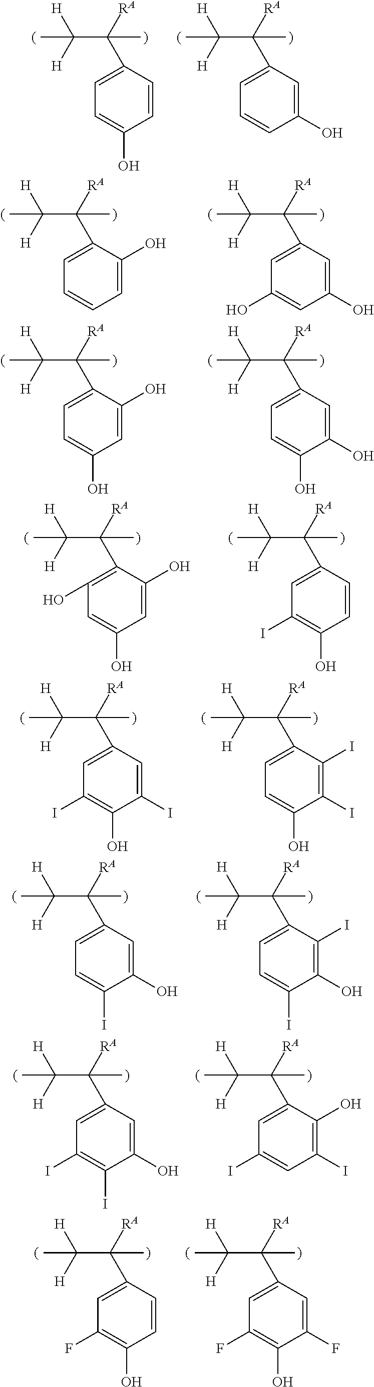

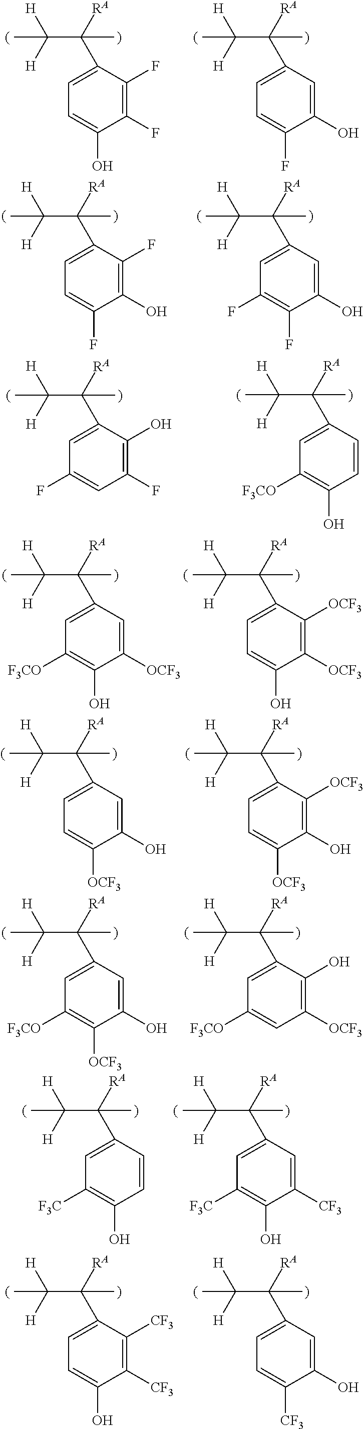

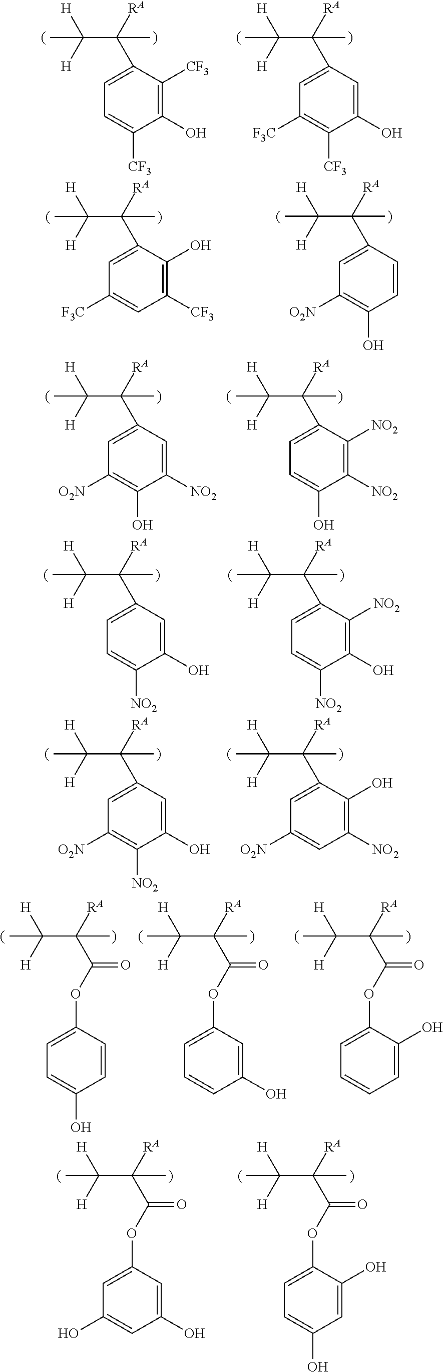

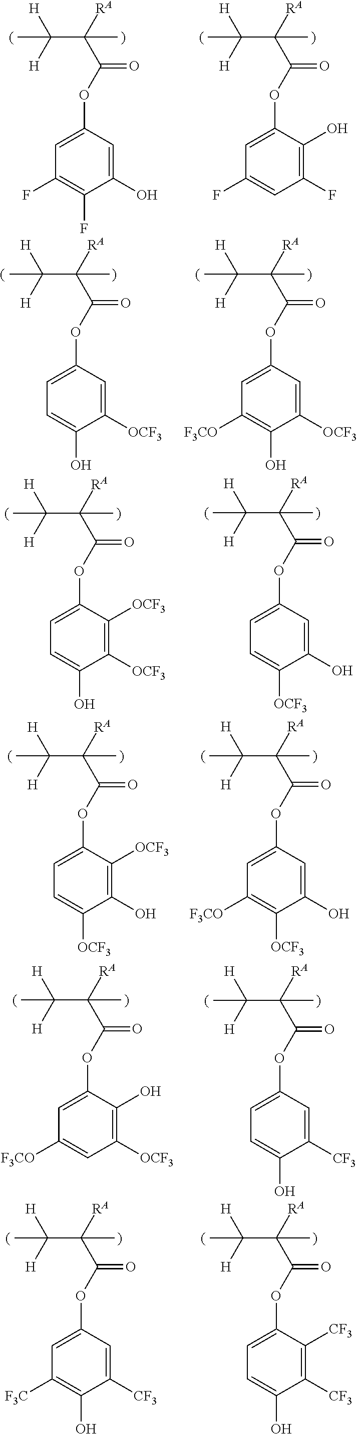

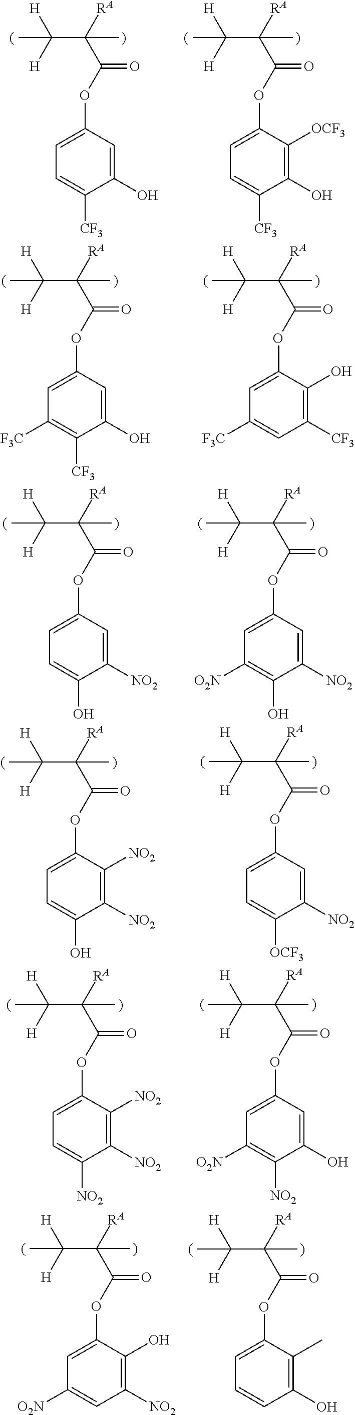

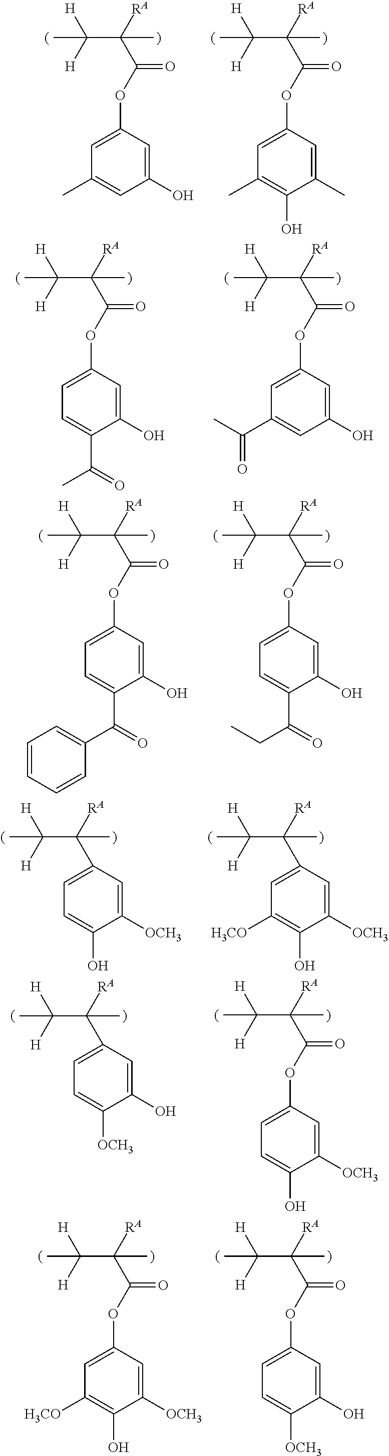

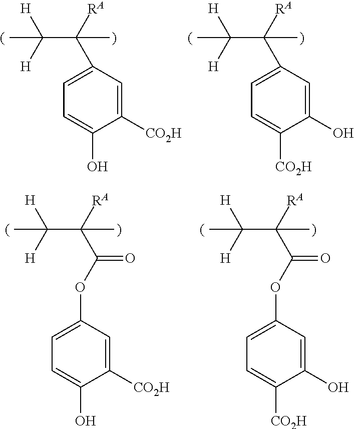

Of the repeat units (b1) or (b2), those units having a lactone ring as the polar group are particularly preferred in the ArF lithography and those units having a phenolic site are preferred in the KrF, EB and EUV lithography.

The base polymer may comprise repeat units of a structure having a hydroxy group protected with an acid labile group, which are also referred to as repeat units (c). The repeat unit (c) is not particularly limited as long as the unit includes one or more structures having a hydroxy group protected with a protective group such that the protective group is decomposed to generate the hydroxy group under the action of acid, but repeat units having the formula (c1) are preferred.

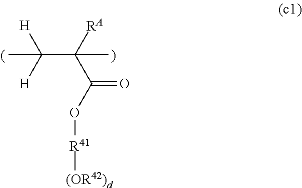

In the formula (c1), R^{A} is hydrogen, fluorine, methyl or trifluoromethyl. R⁴¹ is a C₁-C₃₀ (d+1)-valent hydrocarbon group which may contain a heteroatom. R⁴² is an acid labile group. d is 1, 2, 3, or 4.

In the formula (c1), the acid labile group represented by R⁴² is deprotected under the action of acid so that a hydroxy group is generated. The structure of R⁴² is not particularly limited, but an acetal structure, ketal structure, alkoxycarbonyl group and alkoxymethyl group having the formula (c2) are preferred, with the alkoxymethyl group having the formula (c2) being most preferred.

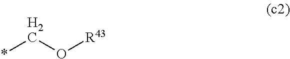

Herein the asterisk (*) denotes a valence bond, and R⁴³ is a C₁-C₁₅ hydrocarbyl group.

Examples of the acid labile group represented by R⁴², the alkoxymethyl group having the formula (c2), and the repeat units (c) are as exemplified for the repeat units (c) in JP-A 2020-111564 (US20200223796).

The base polymer may comprise repeat units (d) derived from indene, benzofuran, benzothiophene, acenaphthylene, chromone, coumarin, norbornadiene or a derivative thereof. Examples of the monomer from which repeat units (d) are derived include those shown below, but not limited thereto.

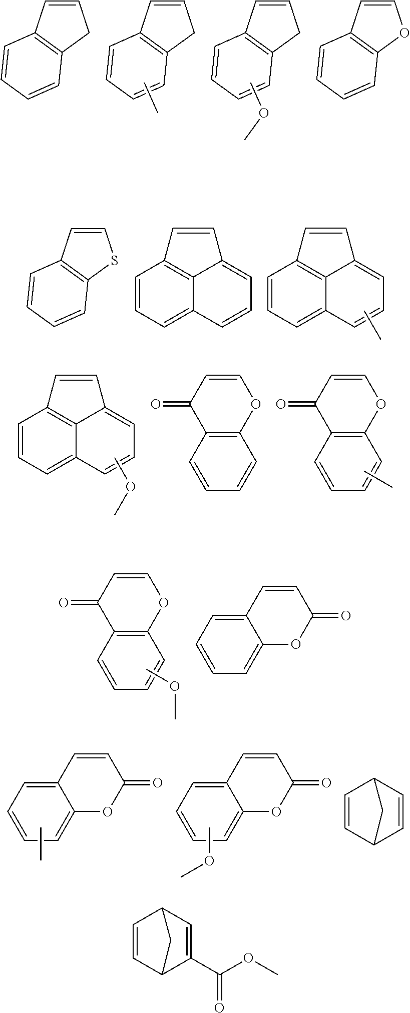

The base polymer may further comprise repeat units (e) derived from indane, vinylpyridine or vinylcarbazole.

In the polymer of the invention, repeat units (A), (a1), (a2), (a3), (b1), (b2), (c), (d), and (e) are incorporated in a ratio of preferably 0<A≤0.4, 0<a1≤0.8, 0≤a2≤0.8, 0≤a3≤0.6, 0<a1+a2+a3≤0.8, 0≤b1≤0.6, 0≤b2≤0.6, 0≤c≤0.5, 0≤d≤0.3, and 0≤e≤0.3; more preferably 0<A≤0.3, 0<a1≤0.7, 0≤a2≤0.7, 0≤a3≤0.5, 0<a1+a2+a3≤0.7, 0≤b1≤0.5, 0≤b2≤0.5, 0≤c≤0.3, 0≤d≤0.3, and 0≤e≤0.3. It is noted that A+a1+a2+a3+b1+b2+c+d+e≤1.0.

The polymer should preferably have a weight average molecular weight (Mw) in the range of 1,000 to 500,000, more preferably 3,000 to 100,000. A Mw in the range ensures that the resist film has sufficient etch resistance and eliminates the risk of resolution decline by a failure to provide a difference in dissolution rate before and after exposure. In the invention, Mw is measured by gel permeation chromatography (GPC) versus polystyrene standards using THF or N,N-dimethylformamide (DMF) solvent.

The influence of the molecular weight distribution (Mw/Mn) of the polymer becomes stronger as the pattern rule becomes finer. Therefore, the polymer should preferably have a narrow dispersity (Mw/Mn) of 1.0 to 2.0 in order to provide a resist composition suitable for micropatterning to a small feature size. A Mw/Mn in the range ensures that the contents of lower and higher molecular weight polymer fractions are low and eliminates a possibility that foreign matter is left on the pattern or the pattern profile is degraded.

The polymer may be synthesized, for example, by dissolving a monomer or monomers corresponding to the above-mentioned repeat units in an organic solvent, adding a radical polymerization initiator, and heating for polymerization.

Examples of the organic solvent which can be used in the polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of initiator added is preferably 0.01 to 25 mol % based on the total of monomers to be polymerized. The reaction temperature is preferably 50 to 150° C., more preferably 60 to 100° C. The reaction time is preferably 2 to 24 hours, more preferably 2 to 12 hours from the aspect of production efficiency.

The polymerization initiator may be fed to the reactor either by adding the initiator to the monomer solution and feeding the solution to the reactor, or by dissolving the initiator in a solvent to form an initiator solution and feeding the initiator solution and the monomer solution separately to the reactor. Because of a possibility that in the standby duration, the initiator generates a radical which triggers polymerization reaction to form an ultra high molecular-weight polymer, it is preferred from the aspect of quality control to prepare the monomer solution and the initiator solution separately and add them dropwise. The acid labile group that has been incorporated in the monomer may be kept as such, or polymerization may be followed by protection or partial protection. Any known chain transfer agent such as dodecyl mercaptan or 2-mercaptoethanol may be added for molecular weight control purpose. The amount of chain transfer agent added is preferably 0.01 to 20 mol % based on the total of monomers to be polymerized.

When a hydroxy-containing monomer is copolymerized, the hydroxy group may be substituted by an acetal moiety which is susceptible to deprotection with acid, typically ethoxyethoxy, prior to polymerization, and the polymerization may be followed by deprotection with weak acid and water. Alternatively, the hydroxy group may be substituted by an acetyl, formyl or pivaloyl moiety prior to polymerization, and the polymerization may be followed by alkaline hydrolysis.

When hydroxystyrene or hydroxyvinylnaphthalene is copolymerized, one method is dissolving hydroxystyrene or hydroxyvinylnaphthalene and other monomers in an organic solvent, adding a radical polymerization initiator thereto, and heating the solution for polymerization. In an alternative method, acetoxystyrene or acetoxyvinylnaphthalene is used instead, and after polymerization, the acetoxy group is deprotected by alkaline hydrolysis, for thereby converting the polymer product to polyhydroxystyrene or hydroxypolyvinylnaphthalene.

For alkaline hydrolysis, a base such as aqueous ammonia or triethylamine may be used. The reaction temperature is preferably-20 to 100° C., more preferably 0 to 60° C. The reaction time is preferably 0.2 to 100 hours, more preferably 0.5 to 20 hours.

The amounts of monomers in the monomer solution may be determined appropriate so as to provide the preferred fractions of repeat units.

It is now described how to use the polymer obtained by the above production method. The reaction solution resulting from polymerization reaction may be used as the final product. Alternatively, the polymer may be recovered in powder form through a purifying step such as re-precipitation step of adding the polymerization solution to a poor solvent and letting the polymer precipitate as powder, after which the polymer powder is used as the final product. It is preferred from the aspects of operation efficiency and consistent quality to handle a polymer solution which is obtained by dissolving the powder polymer resulting from the purifying step in a solvent, as the final product.

The solvents which can be used herein are described in JP-A 2008-111103, paragraphs [0144]-[0145] (U.S. Pat. No. 7,537,880). Exemplary solvents include ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; alcohols such as diacetone alcohol (DAA); high-boiling alcohols such as diethylene glycol, propylene glycol, glycerol, 1,4-butanediol, and 1,3-butanediol; and mixtures thereof.

The polymer solution preferably has a polymer concentration of 0.01 to 30 wt %, more preferably 0.1 to 20 wt %.

Prior to use, the reaction solution or polymer solution is preferably filtered through a filter. Filtration is effective for consistent quality because foreign matter and gel which can cause defects are removed.

Suitable materials of which the filter used for filtration is made include fluorocarbon, cellulose, nylon, polyester, and hydrocarbon base materials. Preferred for the filtration of a resist composition are filters made of fluorocarbons commonly known as Teflon®, hydrocarbons such as polyethylene and polypropylene, and nylon. While the pore size of the filter may be selected appropriate to comply with the desired cleanness, the filter preferably has a pore size of up to 100 nm, more preferably up to 20 nm. A single filter may be used or a plurality of filters may be used in combination. Although the filtering method may be single pass of the solution, preferably the filtering step is repeated by flowing the solution in a circulating manner. In the polymer production process, the filtering step may be carried out any times, in any order and in any stage. The reaction solution as polymerized or the polymer solution may be filtered, preferably both are filtered.

### Chemically Amplified Resist Composition

### (A) Base Polymer

The chemically amplified resist composition of the invention comprises a base polymer comprising the above-described polymer as component (A).

The polymer may be used alone or as a combination of two or more polymers which differ in compositional ratio, Mw and/or Mw/Mn. The base polymer (A) may also comprise a hydrogenated ring-opened metathesis polymer in addition to the polymer. For the ring-opening metathesis polymer, one described in JP-A 2003-066612 can be used.

### (B) Organic Solvent

The chemically amplified resist composition of the invention may comprise an organic solvent as component (B). The organic solvent (B) is not particularly limited as long as the components described above and below are soluble therein. Examples of the organic solvent include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; keto-alcohols such as DAA; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; and mixtures thereof.

Of the foregoing organic solvents, 1-ethoxy-2-propanol, PGMEA, cyclohexanone, GBL, ethyl lactate, DAA and mixtures thereof are preferred because the base polymer (A) is most soluble therein.

The amount of the organic solvent (B) in the chemically amplified resist composition of the invention is preferably 200 to 7,000 parts by weight, more preferably 400 to 5,000 parts by weight per 80 parts by weight of the base polymer (A). The organic solvent (B) may be used alone or in admixture of two or more.

### (C) Quencher

The chemically amplified resist composition of the invention may comprise a quencher as component (C). As used herein, the term “quencher” refers to a compound capable of trapping the acid generated by a photoacid generator in the chemically amplified resist composition to prevent the acid from diffusing to the unexposed region and to assist in forming the desired pattern.

Examples of the quencher (C) include an onium salt having the formula (1) or (2).

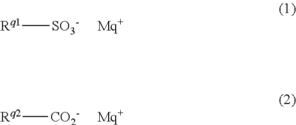

In the formula (1), R⁹¹ is hydrogen or a C₁-C₄₀ hydrocarbyl group which may contain a heteroatom, exclusive of the group wherein hydrogen bonded to the carbon atom at α-position relative to the sulfo group is substituted by fluorine or fluoroalkyl. In the formula (2), R⁴² is hydrogen or a C₁-C₄₀ hydrocarbyl group which may contain a heteroatom.

Examples of the C₁-C₄₀ hydrocarbyl group represented by R⁴¹ include C₁-C₄₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decyl, and adamantyl; C₆-C₄₀ aryl groups such as phenyl, naphthyl and anthracenyl. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

Examples of the hydrocarbyl group represented by R⁴² include substituents exemplified above for R⁹¹ as well as fluorinated saturated hydrocarbyl groups such as trifluoromethyl and trifluoroethyl, and fluorinated aryl groups such as pentafluorophenyl and 4-trifluoromethylphenyl.

Examples of the anion in the onium salt having the formula (1) include those shown below, but not limited thereto.

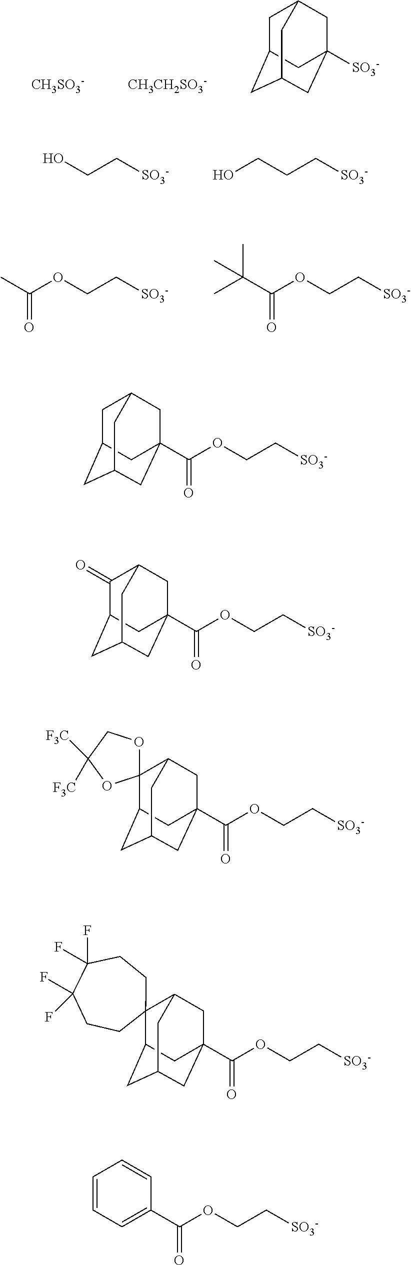

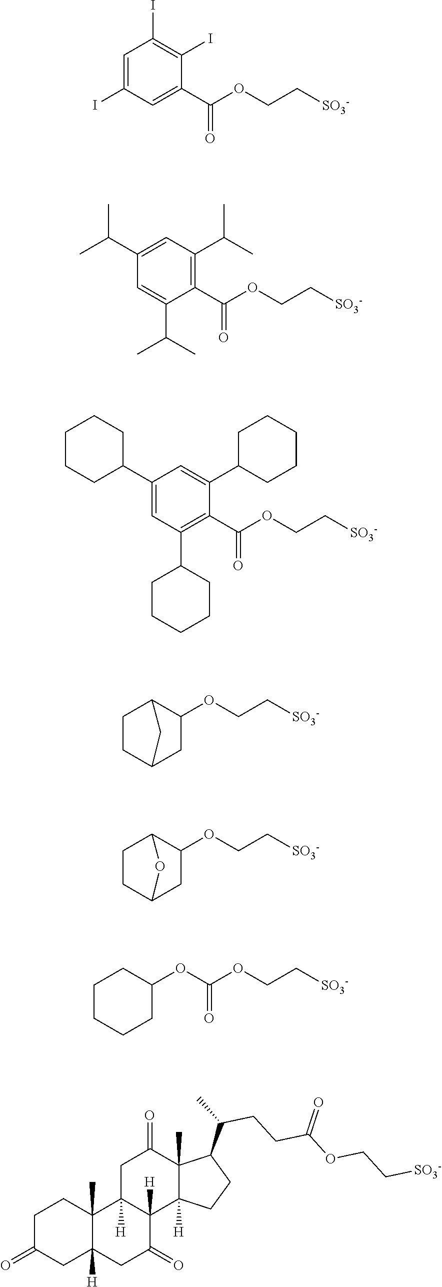

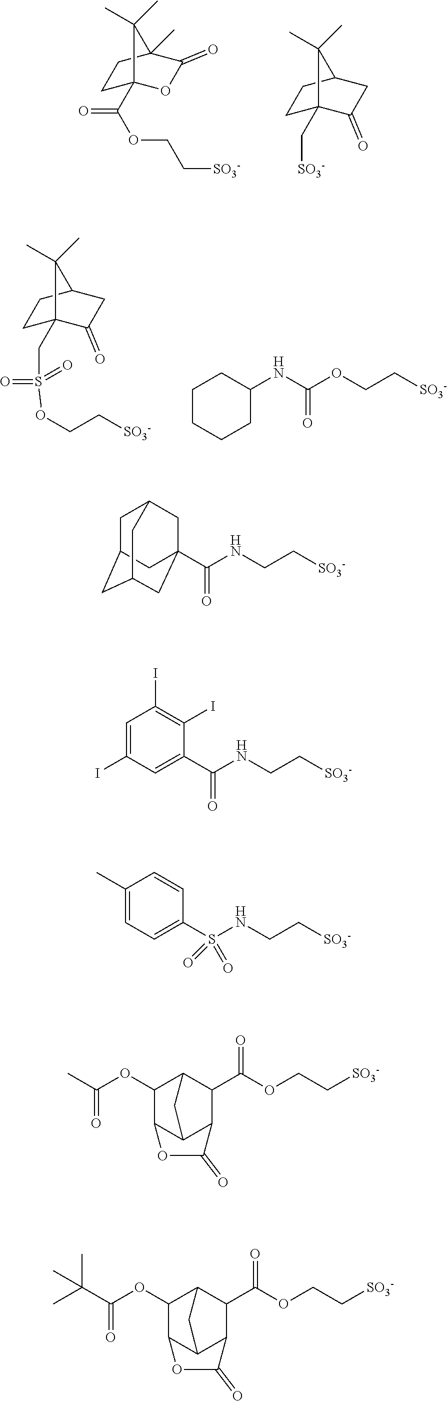

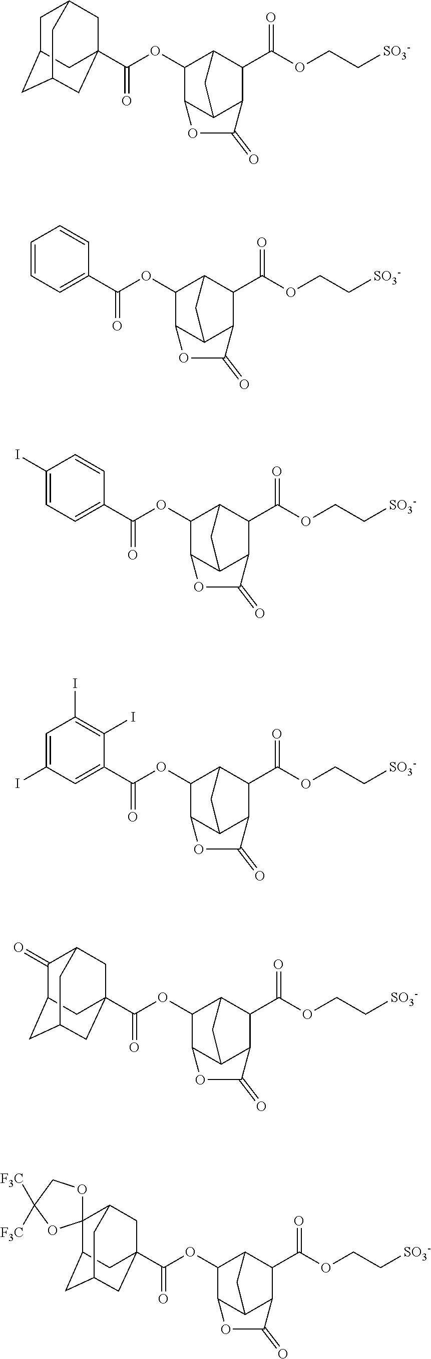

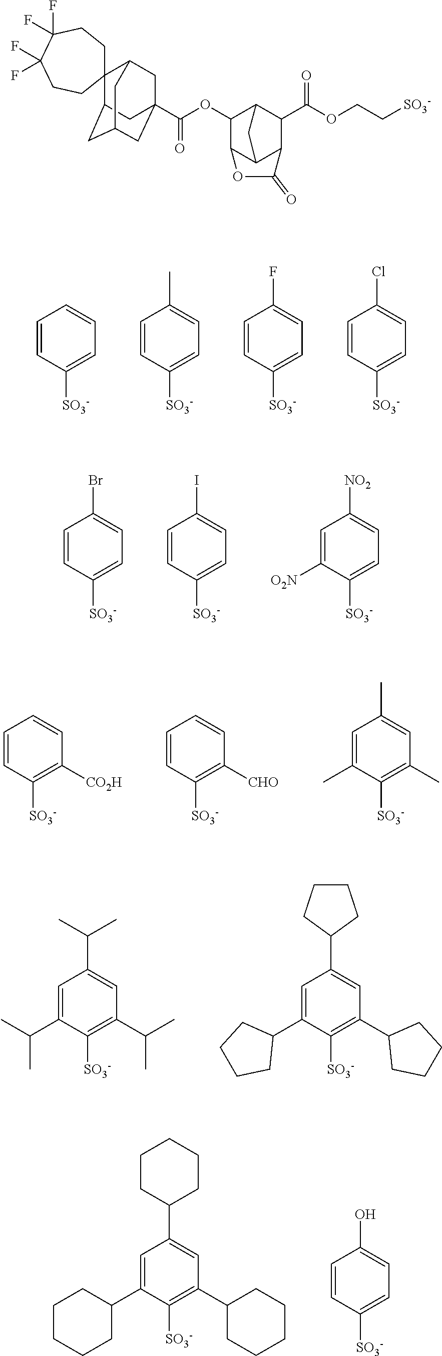

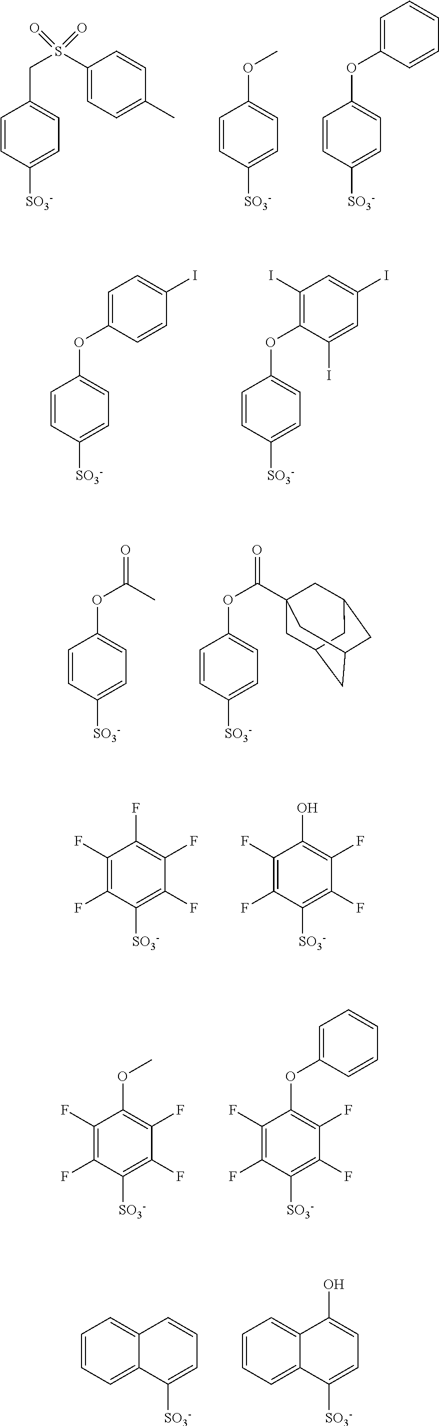

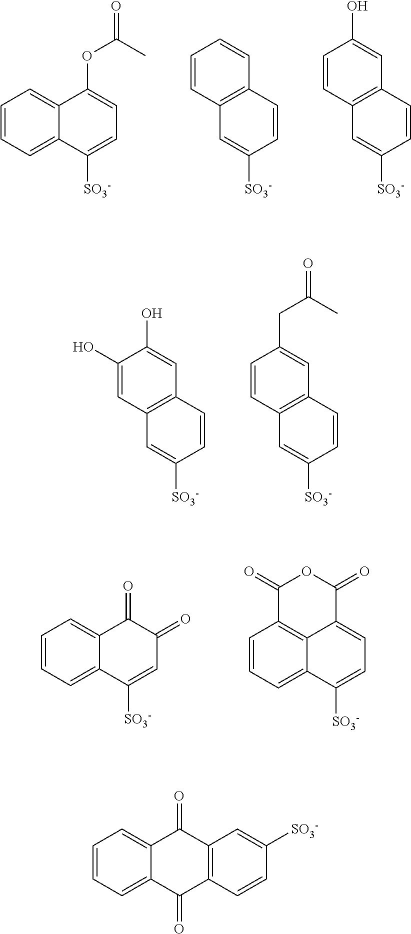

Examples of the anion in the onium salt having the formula (2) include those shown below, but not limited thereto.

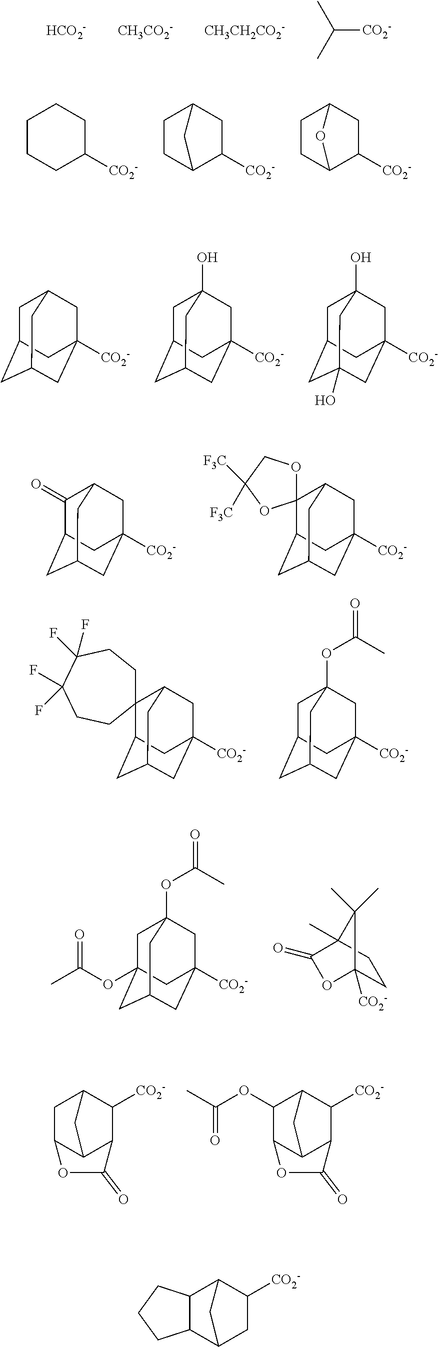

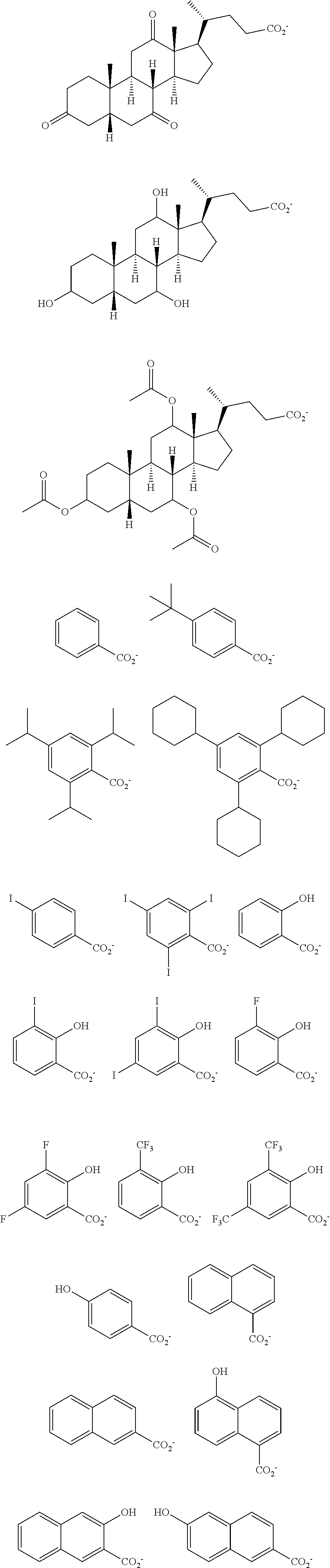

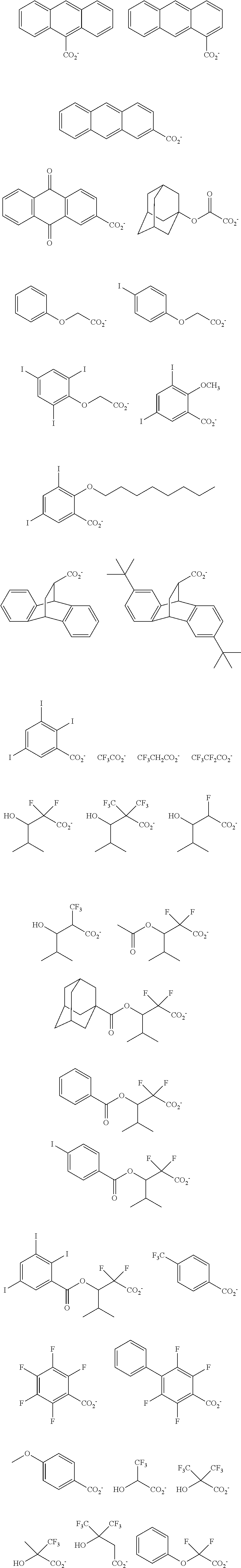

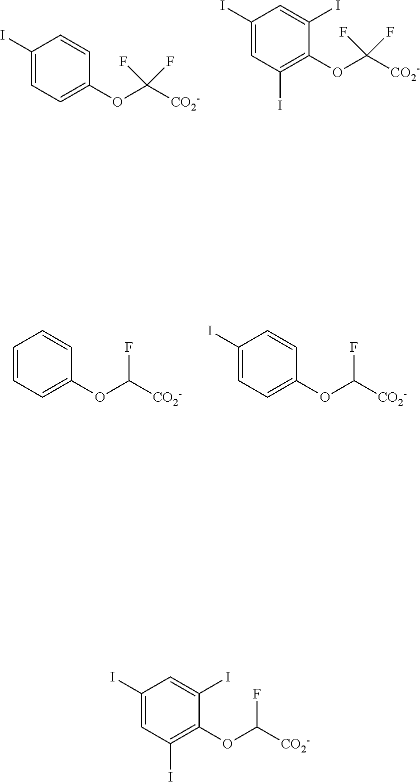

In the formulae (1) and (2), Mq⁺ is an onium cation. Examples of the onium cation include sulfonium, iodonium and ammonium cations. Examples of the sulfonium cation include those described as examples of the sulfonium cation in the formula (A), those described in JP-A 2024-003744, paragraphs to [0125], those described in WO 2024/128017, paragraphs to [0049], and those described in JP 7491173, paragraphs to [0046], but not limited thereto.

As the sulfonium cation, a sulfonium cation having the formula (sulfo-1) is also preferred.

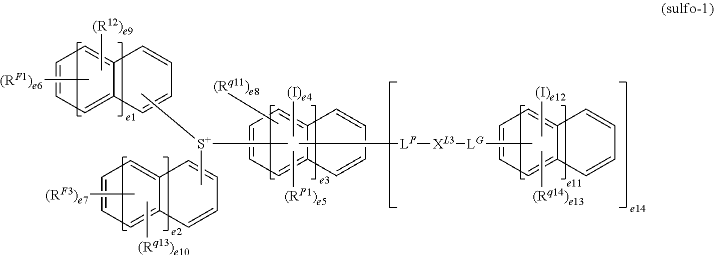

In the formula (sulfo-1), e1 is 0 or 1. The relevant structure is a benzene ring in case of e1=0, and a naphthalene ring in case of e1=1. The benzene ring corresponding to e1=0 is preferred from the aspect of solvent solubility. e2 is 0 or 1. The relevant structure is a benzene ring in case of e2=0, and a naphthalene ring in case of e2=1. The benzene ring corresponding to e2-0 is preferred from the aspect of solvent solubility. e3 is 0 or 1. The relevant structure is a benzene ring in case of e3-0, and a naphthalene ring in case of e3=1. The benzene ring corresponding to e3=0 is preferred from the aspect of solvent solubility.

In the formula (sulfo-1), e4 is 0, 1, 2, 3, or 4. A cation structure containing more iodine atoms is more absorptive to EUV, but loses solvent solubility so that it may precipitate in a resist composition, e4 is preferably 0, 1, 2, or 3, more preferably 0, 1, or 2.

In the formula (sulfo-1), e5 is 0, 1, 2, 3, or 4. e5 is preferably 0, 1, 2, or 3, more preferably 0, 1, or 2 from the aspect of availability of reactants. e6 is 0, 1, 2, 3, 4, 5, or 6. e6 is preferably 0, 1, 2, or 3, more preferably 0, 1, or 2 from the aspect of availability of reactants. e7 is 0, 1, 2, 3, 4, 5, or 6. e7 is preferably 0, 1, 2, or 3, more preferably 0, 1, or 2 from the aspect of availability of reactants.

In the formula (sulfo-1), e8 is 0, 1, or 2. e8 is preferably 0 or 1 from the aspect of availability of reactants. e9 is 0, 1, or 2. e9 is preferably 0 or 1 from the aspect of availability of reactants. e10 is 0, 1, or 2. e10 is preferably 0 or 1 from the aspect of availability of reactants.

In the formula (sulfo-1), e11 is 0 or 1. The relevant structure is a benzene ring in case of e11=0, and a naphthalene ring in case of e11=1. The benzene ring corresponding to e11=0 is preferred from the aspect of solvent solubility.

In the formula (sulfo-1), e12 is 0, 1, 2, 3, or 4. A cation structure containing more iodine atoms is more absorptive to EUV, but loses solvent solubility so that it may precipitate in a resist composition, e12 is preferably 0, 1, 2, or 3, more preferably 0, 1, or 2.

In the formula (sulfo-1), e13 is 0, 1, or 2. e13 is preferably 0 or 1 from the aspect of availability of reactants. e14 is 0, 1, or 2. From the aspect of synthesis, e14 is preferably 0 or 1.

It is noted that 0≤e6+e9≤4 in case of e1=0 and 0≤e6+e9≤6 in case of e1=1; 0≤e7+e10≤4 in case of e2-0 and 0≤e7+e10≤6 in case of e2=1; 1≤e4+e5+e8+e14≤4 in case of e3-0 and 1≤e4+e5+e8+e14≤6 in case of e3=1; 0≤e12+e13≤4 in case of e11=0 and 0≤e12+e13≤6 in case of e11=1; and e4+e12≥1.

In the formula (sulfo-1), R^{F1} to R^{F3} are each independently fluorine, a C₁-C₆ fluorinated saturated hydrocarbyl group, a C₁-C₆ fluorinated saturated hydrocarbyloxy group, or a C₁-C₆ fluorinated saturated hydrocarbylthio group. Among them, R^{F1} to R^{F3} are preferably a trifluoromethyl, trifluoromethoxy or trifluorothiomethoxy group. A plurality of RFI may be identical or different when e5 is 2, 3, or 4, a plurality of R^{F2} may be identical or different when e6 is 2, 3, 4, 5, or 6, and a plurality of R^{F3} may be identical or different when e7 is 2, 3, 4, 5, or 6.

In the formula (sulfo-1), R^{q11} to R^{q14} are halogen exclusive of iodine and fluorine, a nitro group, a cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. The hydrocarbyl group and hydrocarbyl moiety in the hydrocarbyloxy and hydrocarbylthio groups may be saturated or unsaturated and straight, branched or cyclic. Examples thereof include those exemplified above for the hydrocarbyl group represented by R¹ in the formula (A). In the hydrocarbyl group and hydrocarbyl moiety in the hydrocarbyloxy and hydrocarbylthio groups, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety.

When e8 is 2, two R^{q11} may be identical or different and two R^{q11} may bond together to form a ring with the carbon atoms to which they are attached; when e9 is 2, two R^{q12} may be identical or different and two R⁹¹² may bond together to form a ring with the carbon atoms to which they are attached; when e10 is 2, two R^{q13} may be identical or different and two R^{q13} may bond together to form a ring with the carbon atoms to which they are attached; and when e13 is 2, two R^{q14} may be identical or different and two R^{q14} may bond together to form a ring with the carbon atoms to which they are attached. Examples of the ring thus formed include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

The aromatic rings directly bonded to S⁺ in the sulfonium cation having the formula (sulfo-1) may bond together to form a ring with S⁺. Examples of the structure of the ring include structures having the formulae:

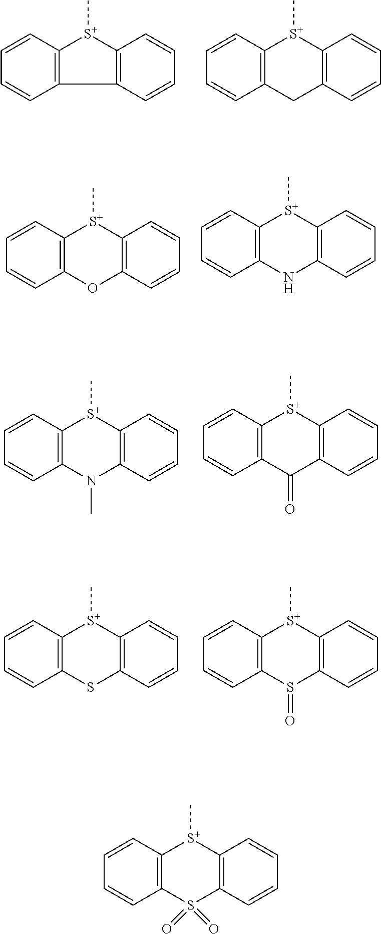

wherein the broken line is a valence bond.

In the formula (sulfo-1), L^{F} and L^{G} are each independently a single bond, ether bond, ester bond, amide bond, sulfonate ester bond, sulfonamide bond, carbonate bond or carbamate bond. Of these, L^{F} is preferably a single bond, ether bond, ester bond or sulfonate ester bond, more preferably an ester bond or sulfonate ester bond. L^{G} is preferably a single bond, ether bond or ester bond, more preferably a single bond.

In the formula (sulfo-1), X¹³ is a single bond or a C₁-C₄₀ hydrocarbylene group which may contain a heteroatom. The hydrocarbylene group may be straight, branched or cyclic, and examples thereof include alkanediyl, cyclic saturated hydrocarbylene and arylene groups. Examples of the heteroatom include oxygen, nitrogen and sulfur atoms. Examples of the C₁-C₄₀ hydrocarbylene group which may contain a heteroatom represented by X¹³ include XL-0 to XL-58 as exemplified for the C₁-C₄₀ hydrocarbylene groups which may contain a heteroatom represented by XLI and X^{L2} in the formula (Z). Of XL-0 to XL-58, X¹³ is preferably XL-0 to XL-22, XL-29 to XL-34, and XL-47 to XL-58.

As the sulfonium cation having the formula (sulfo-1), a sulfonium cation having the formula (sulfo-1-1) is preferred.

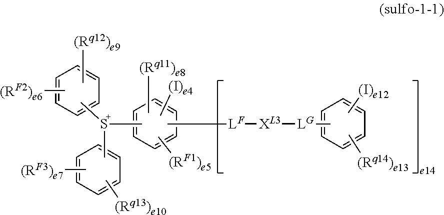

Herein c4 to e10, e12 to e14, R^{F1} to R^{F3}, R^{q11} to R^{q14}, L^{F}, L^{G} and X^{L3} are as defined above.

As the sulfonium cation having the formula (sulfo-1-1), a sulfonium cation having the formula (sulfo-1-2) is preferred.

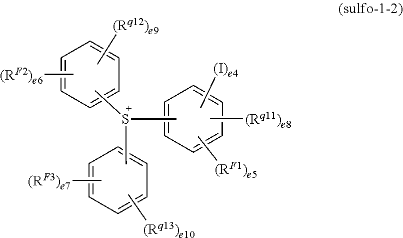

Herein e4 to e10, R^{F1} to R^{F3} and R^{q11} to R^{q13} are as defined above.

Examples of the sulfonium cation represented by the formula (sulfo-1) include those shown below, but not limited thereto. In the formulae. Me is methyl.

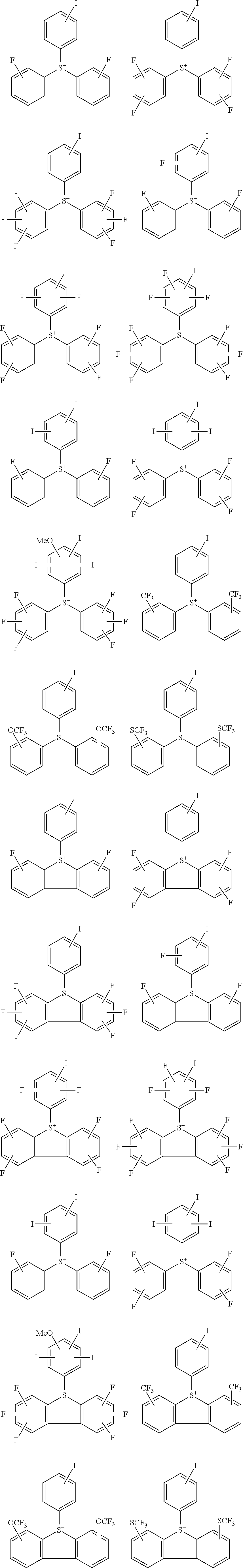

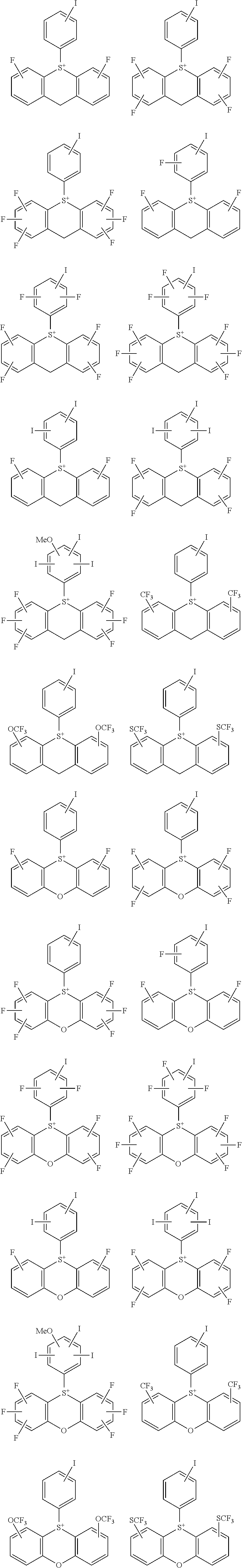

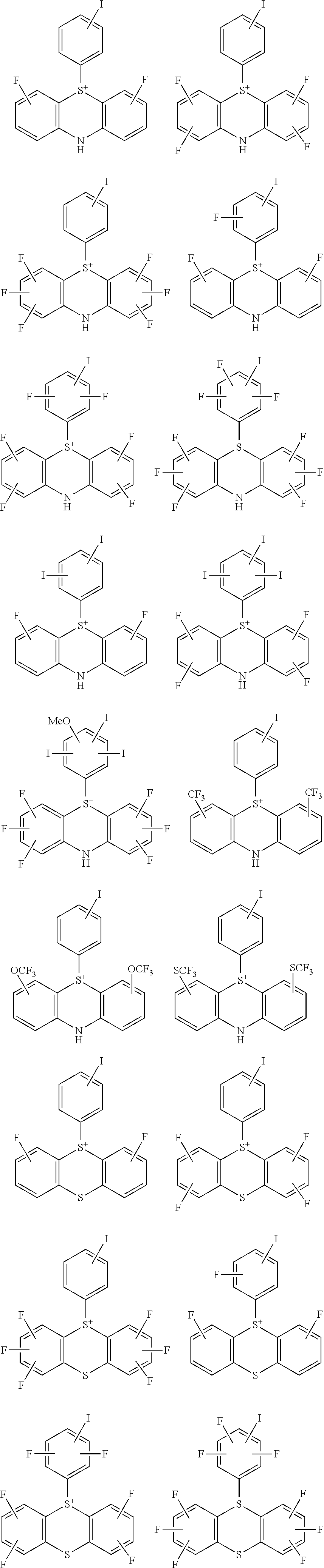

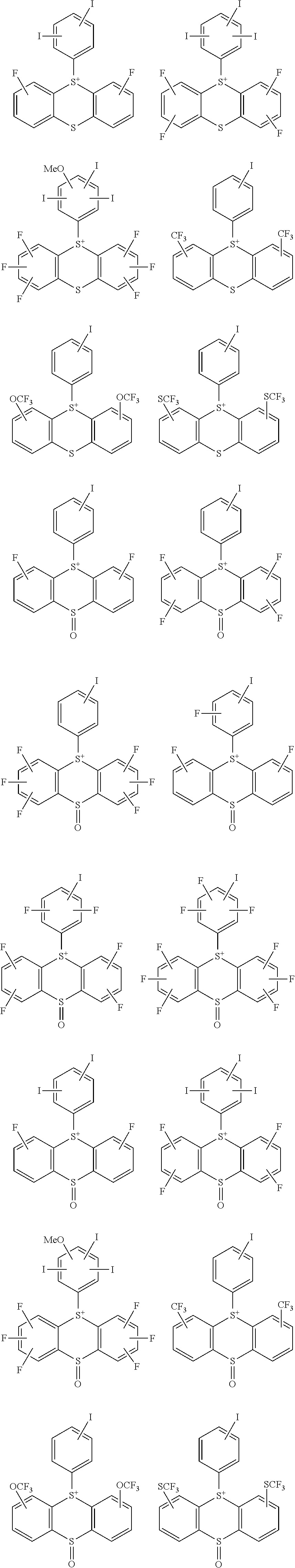

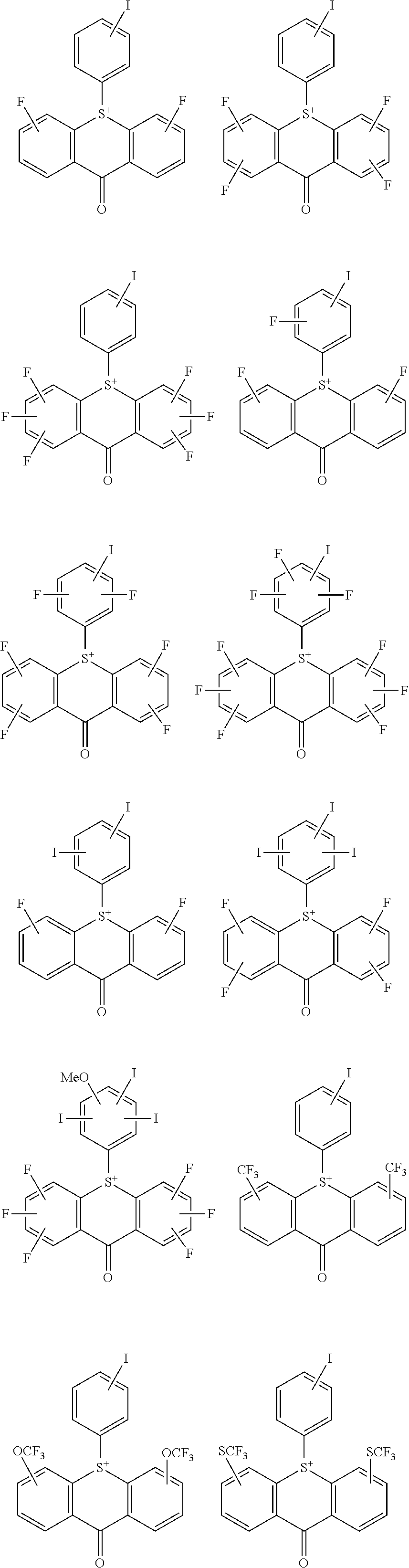

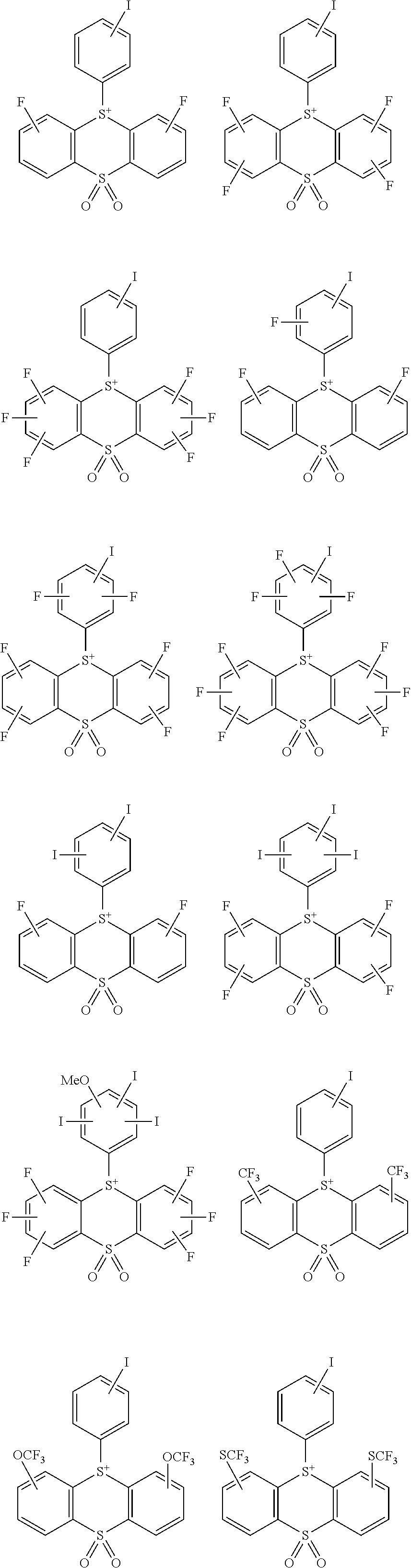

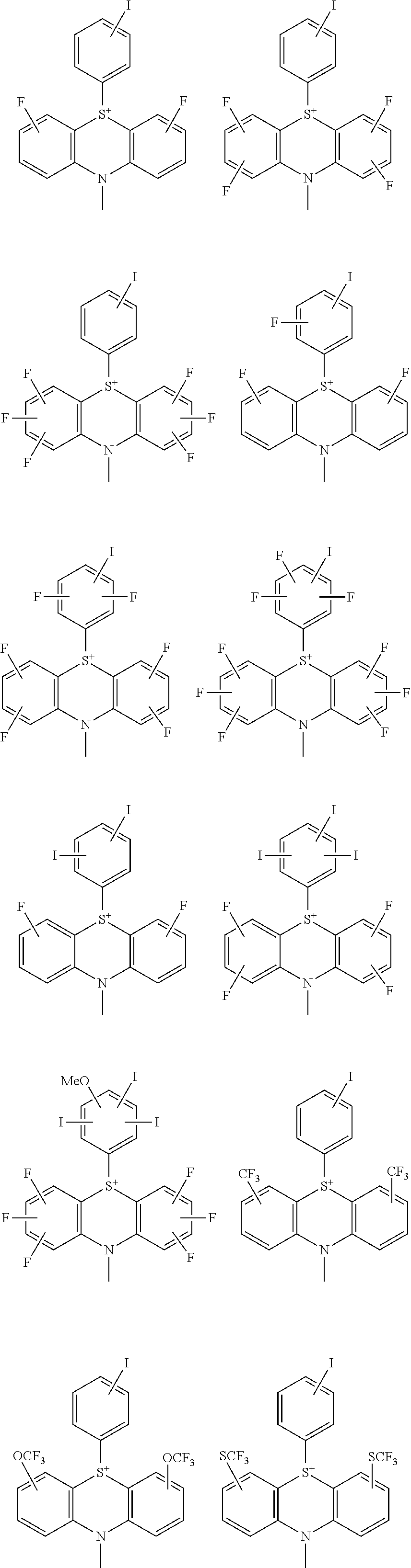

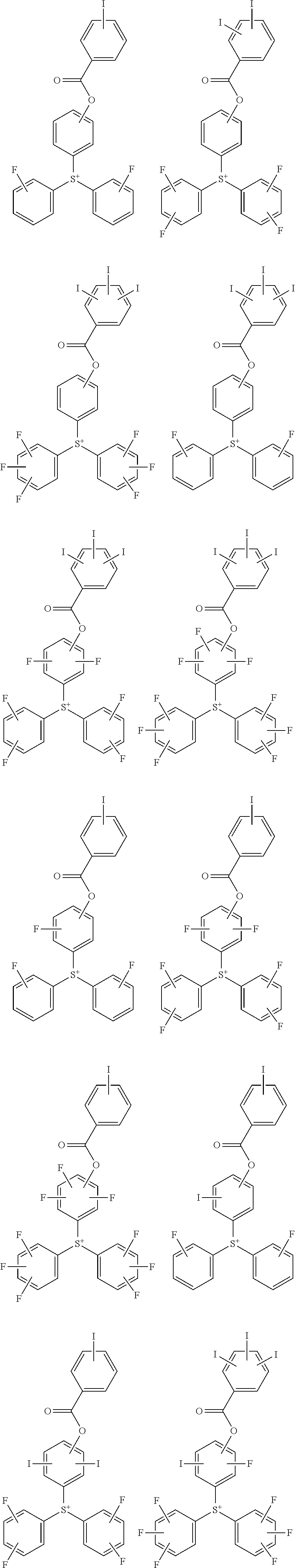

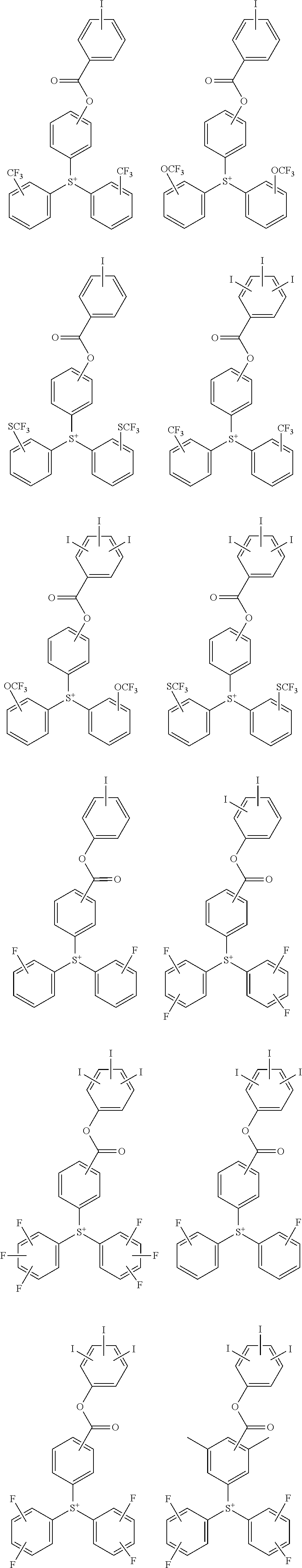

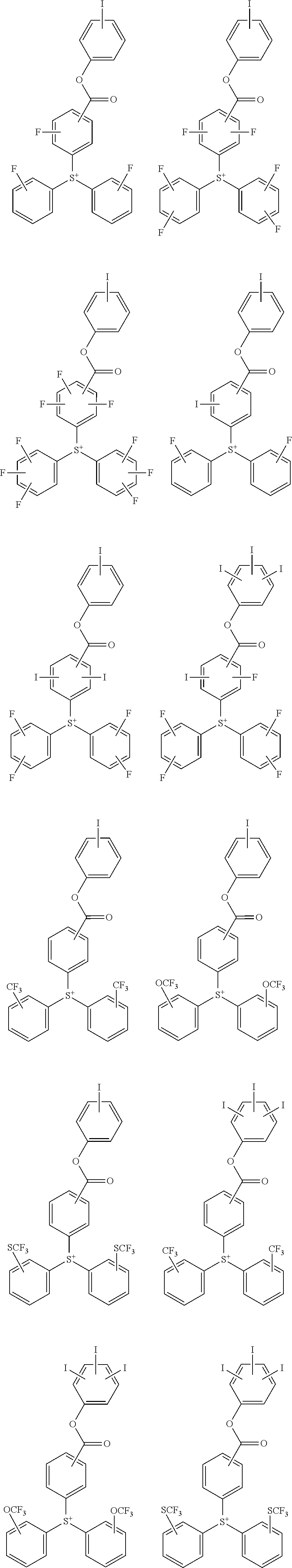

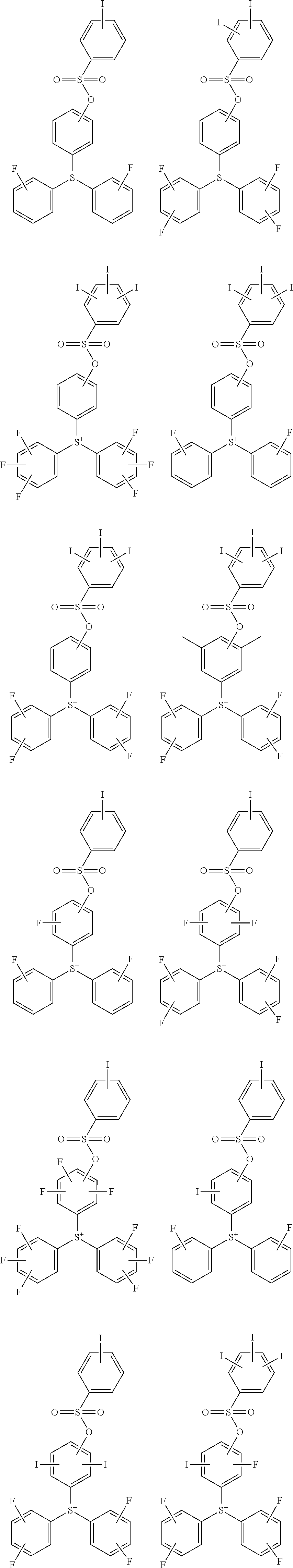

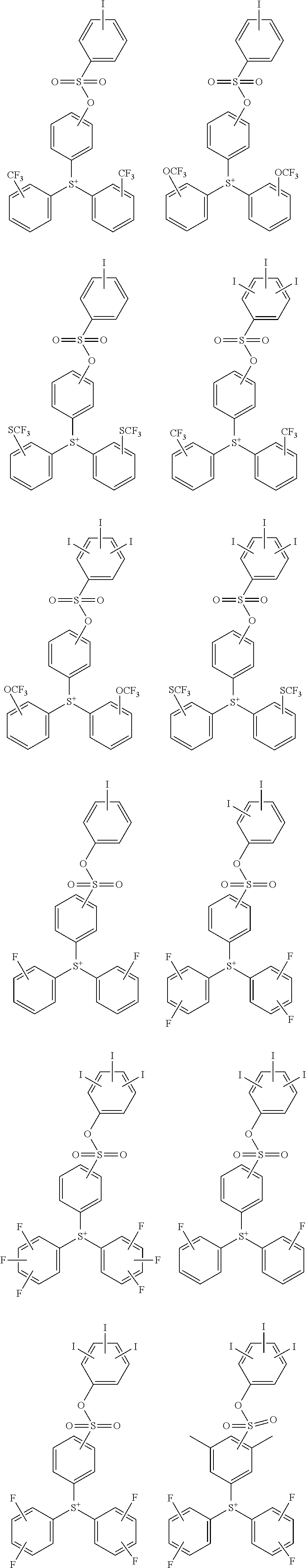

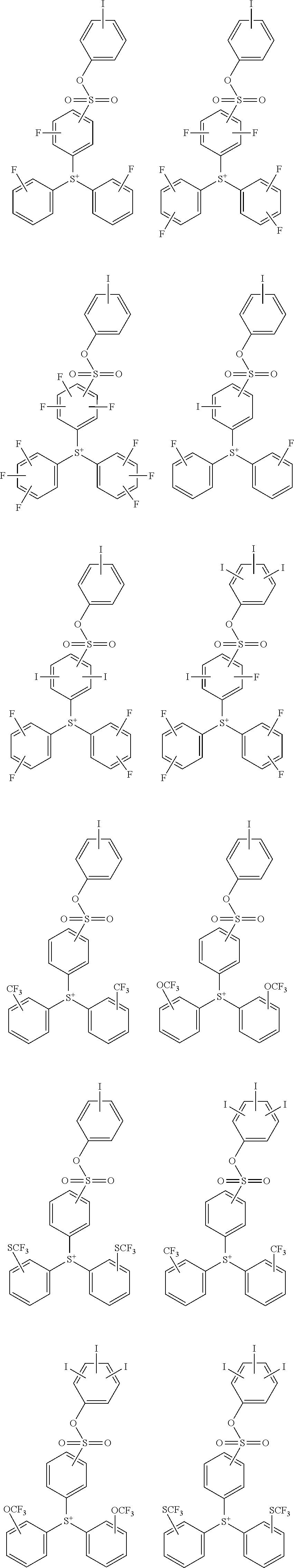

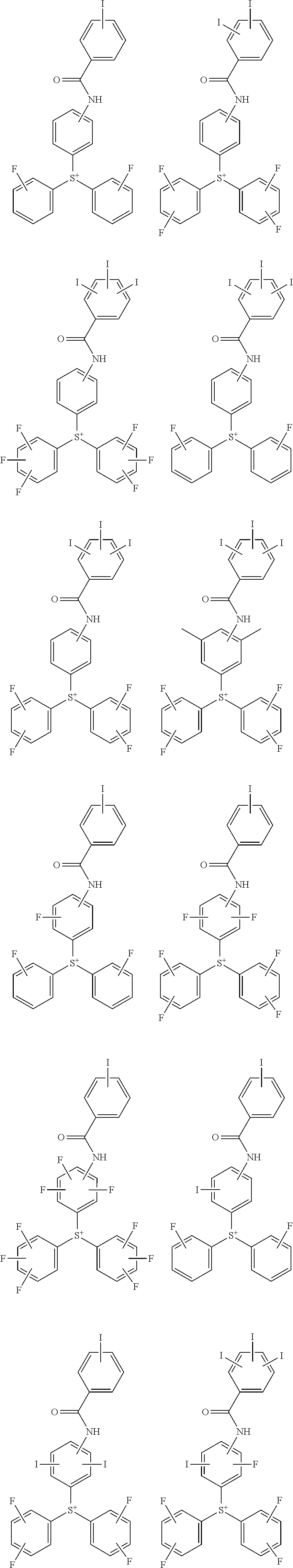

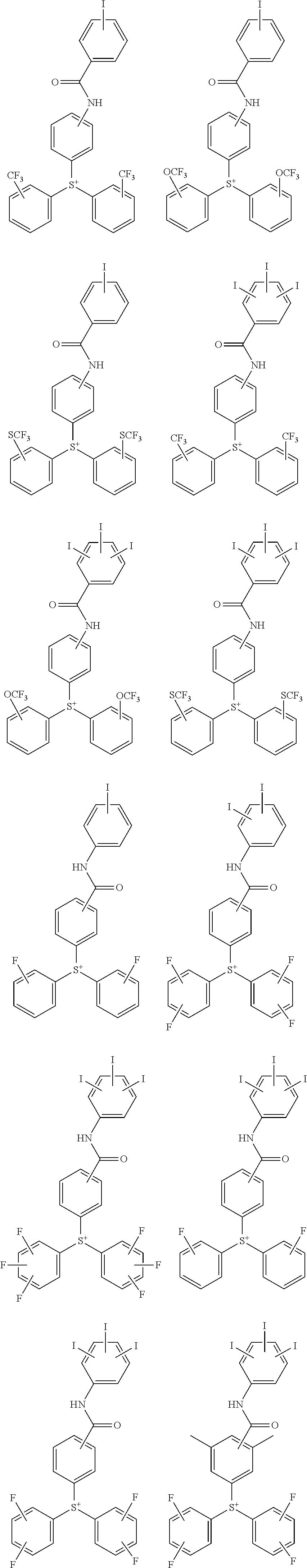

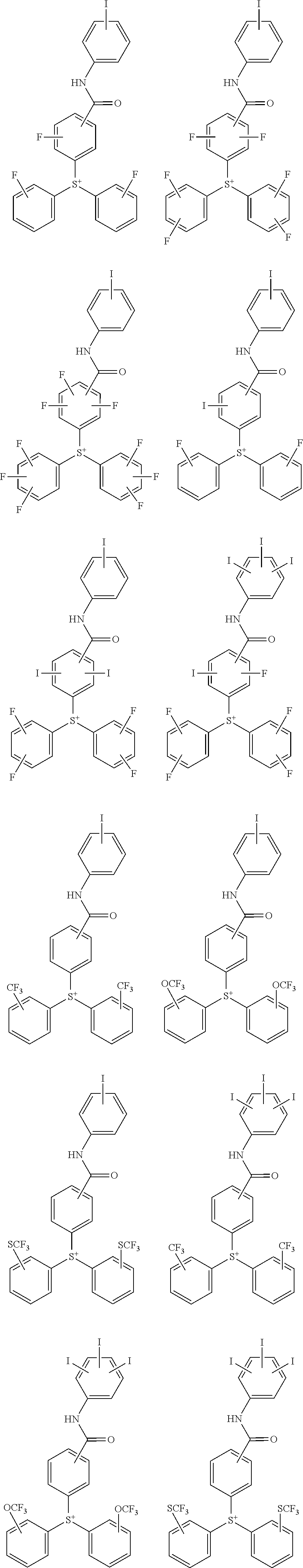

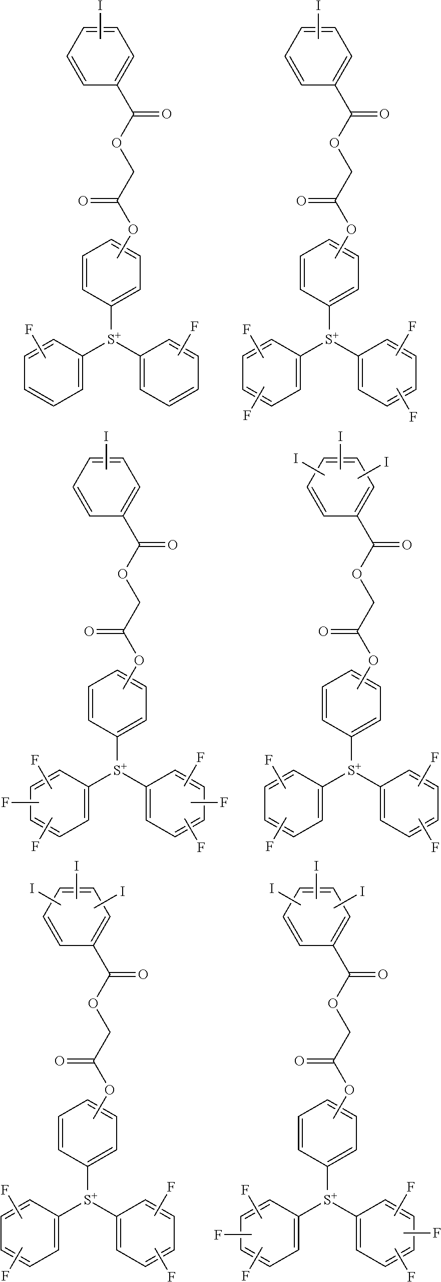

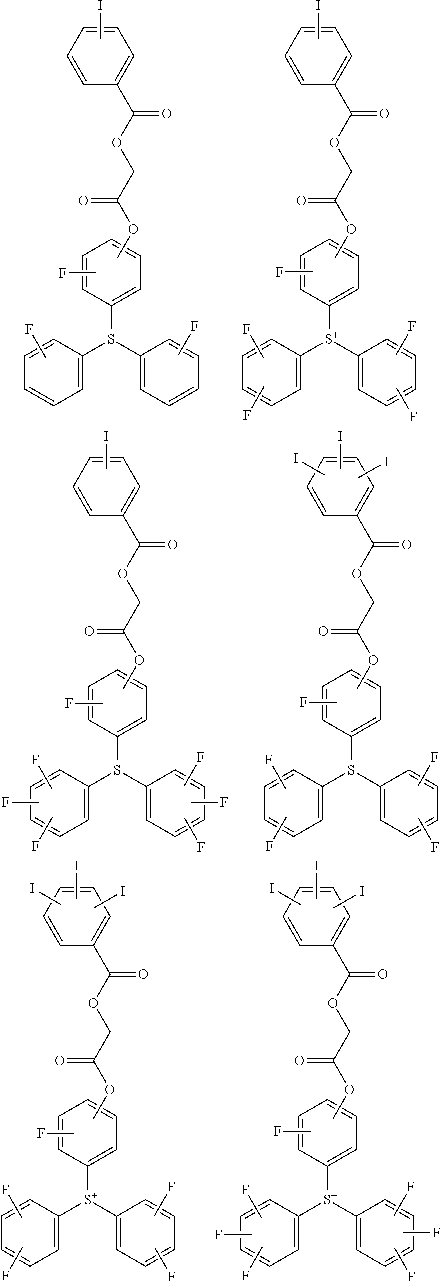

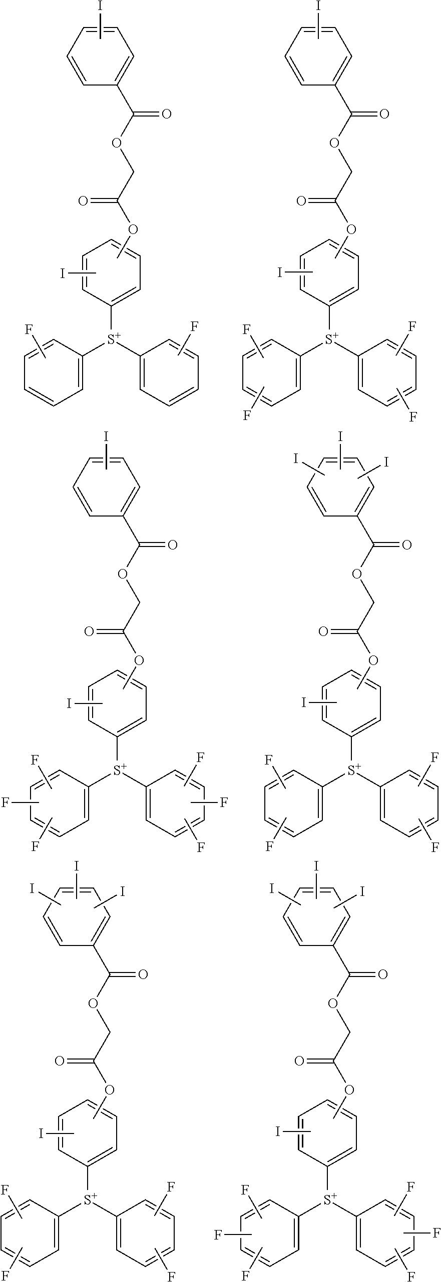

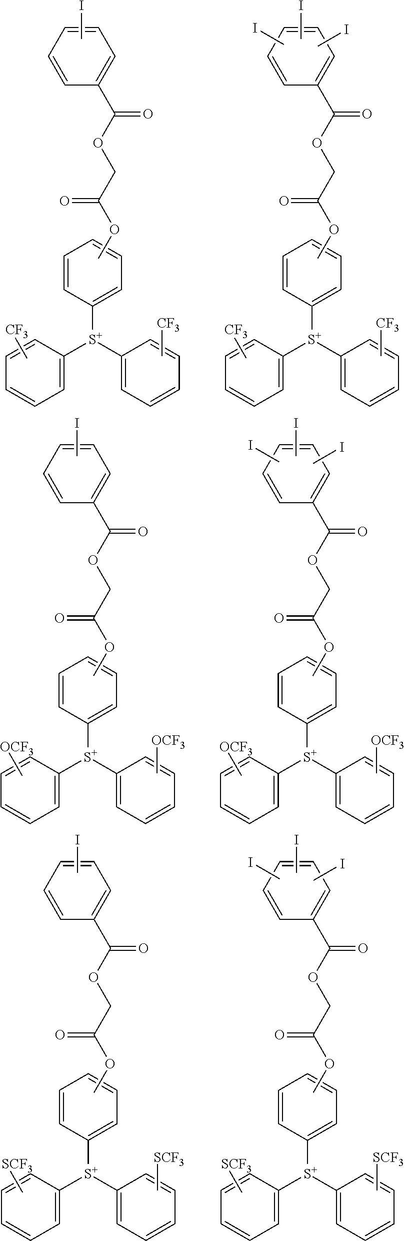

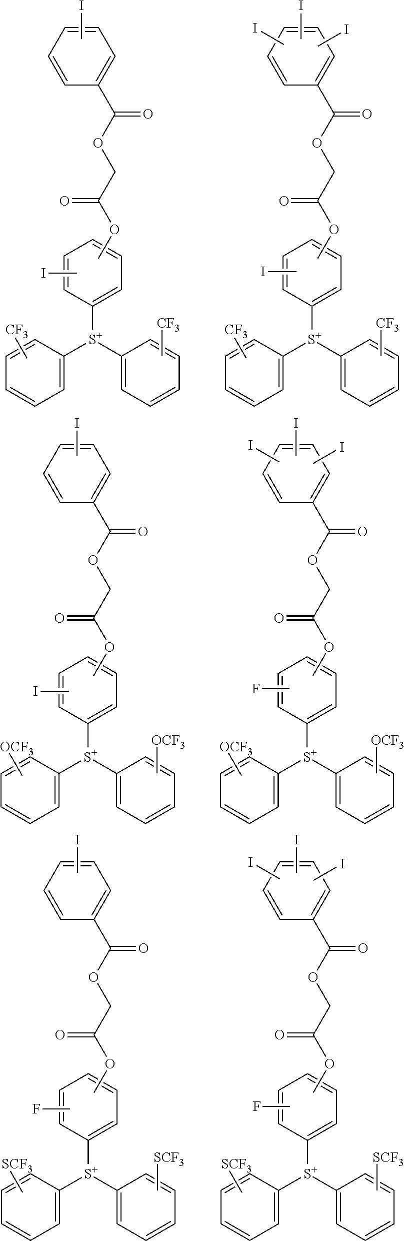

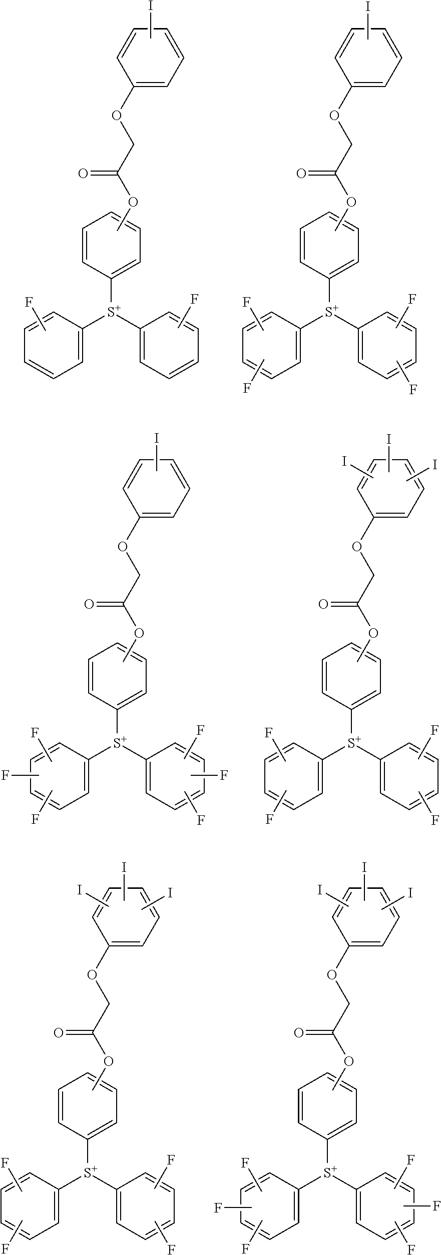

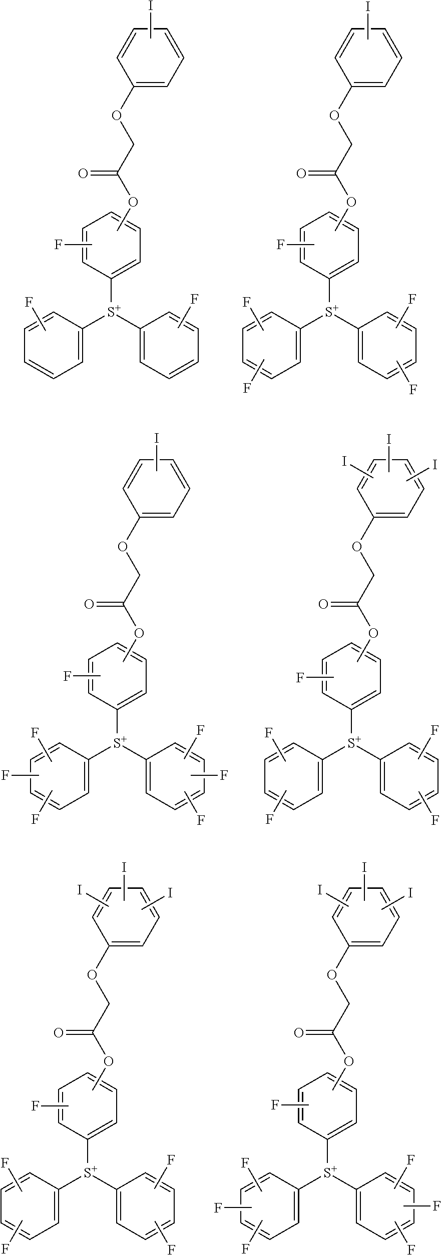

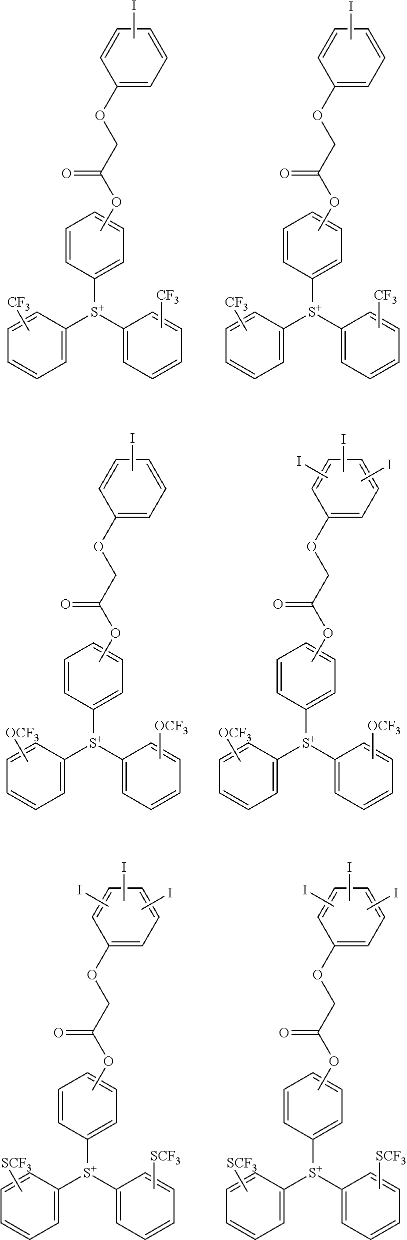

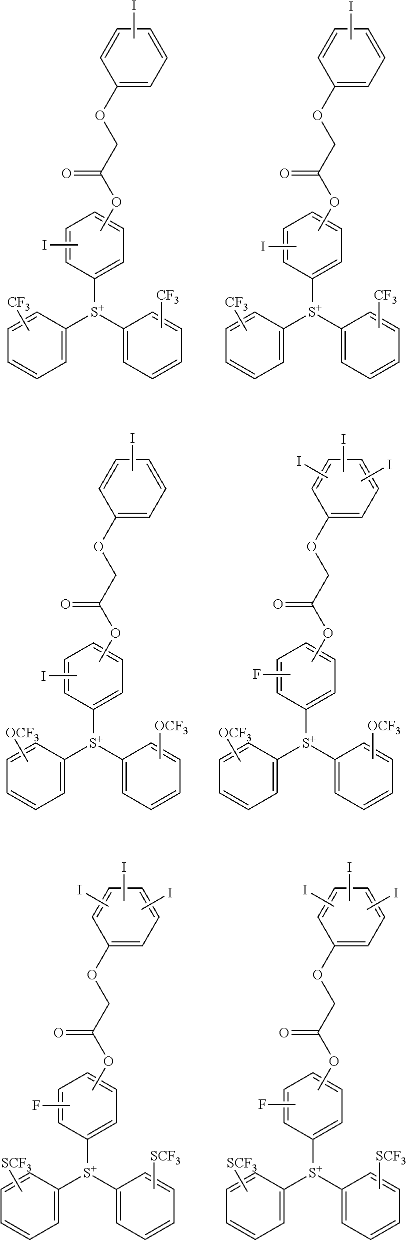

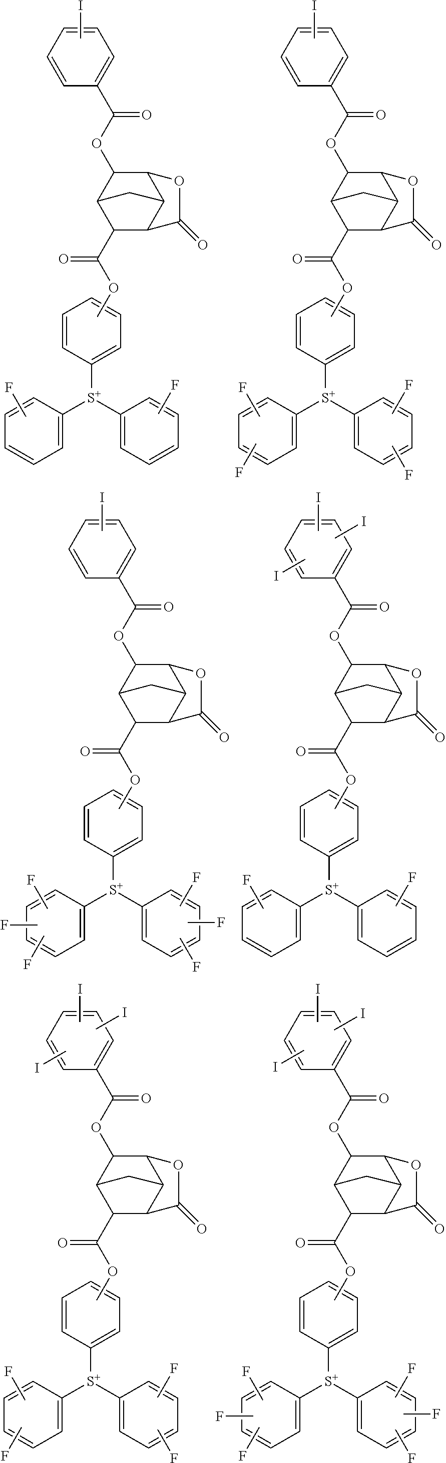

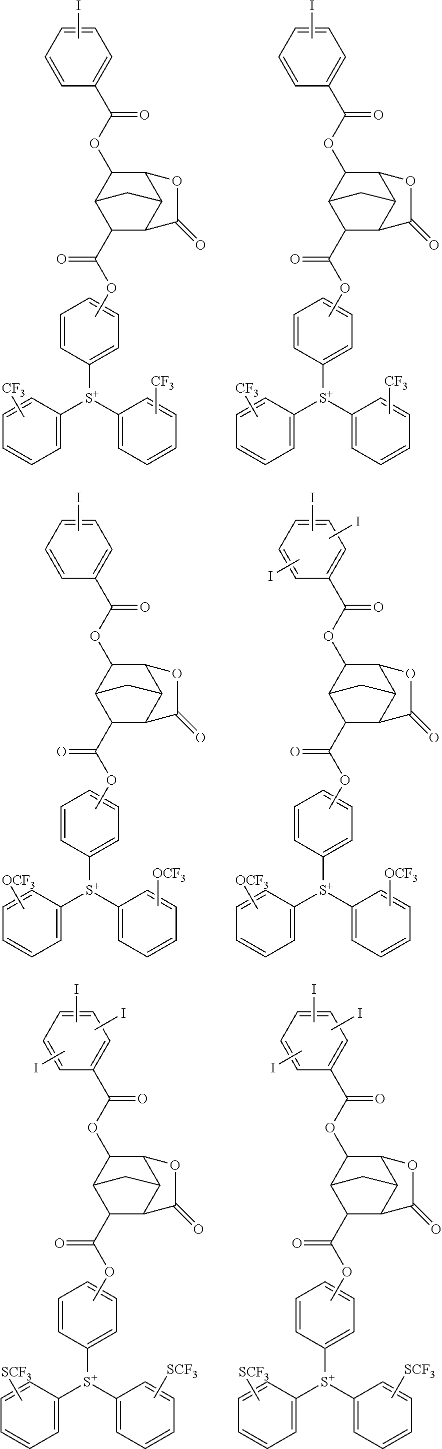

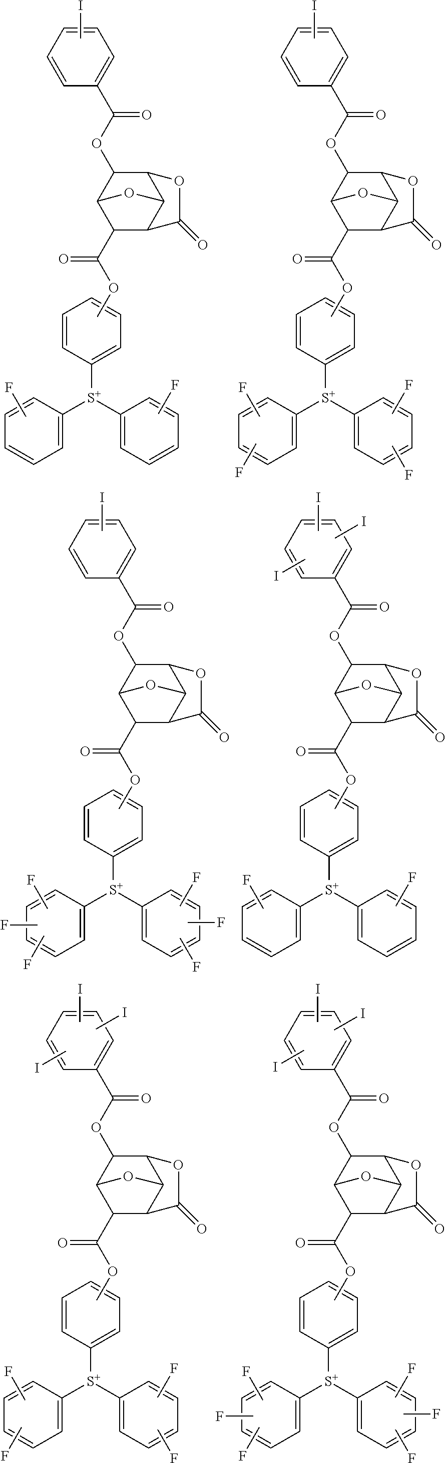

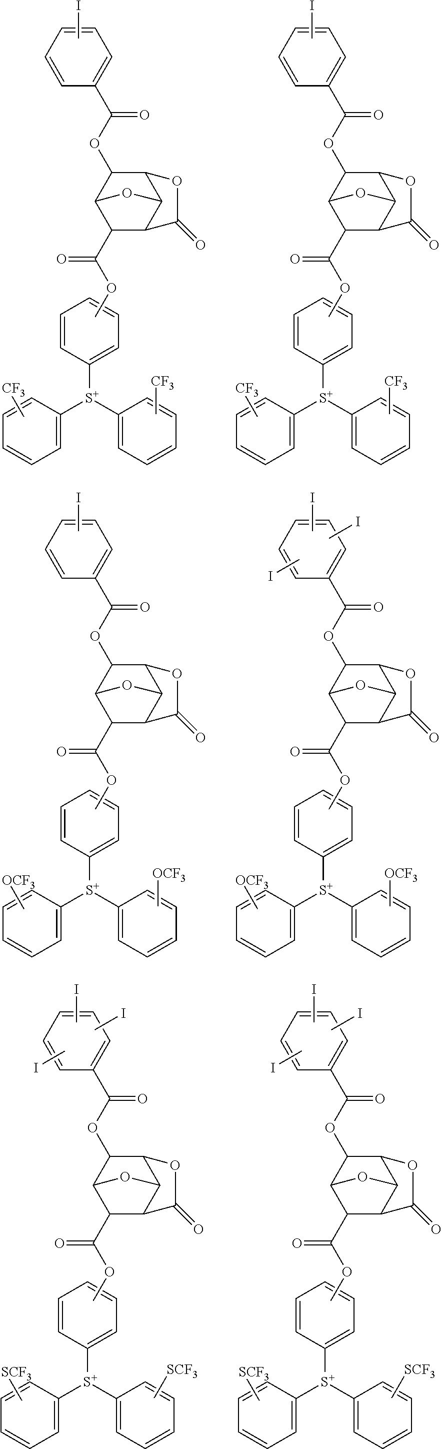

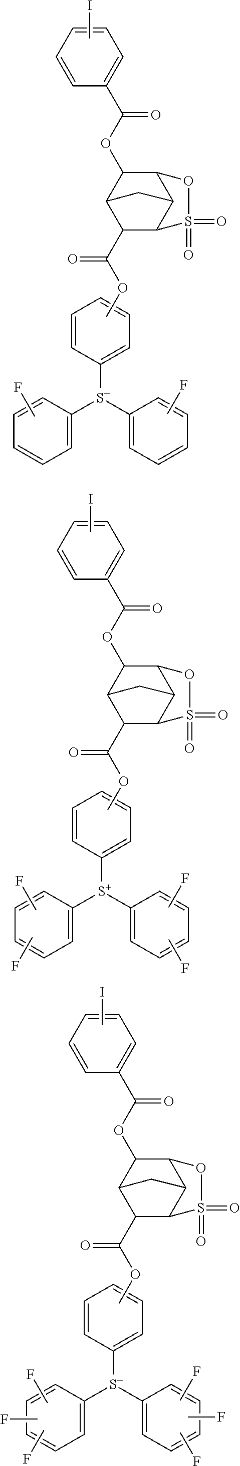

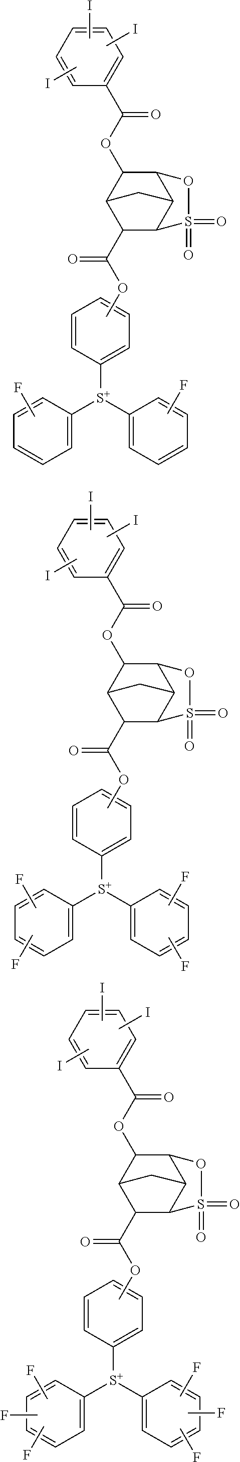

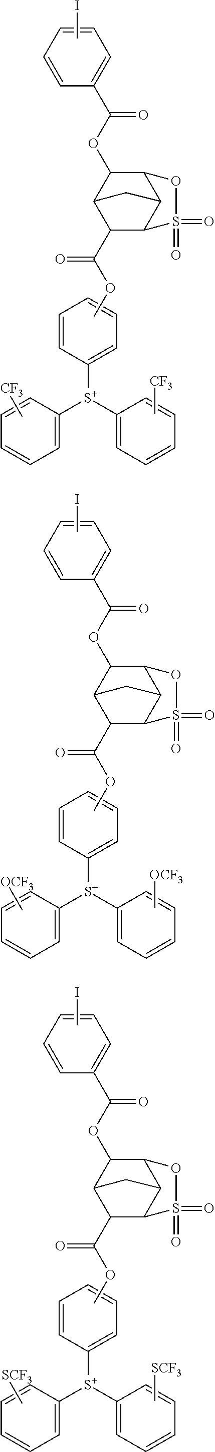

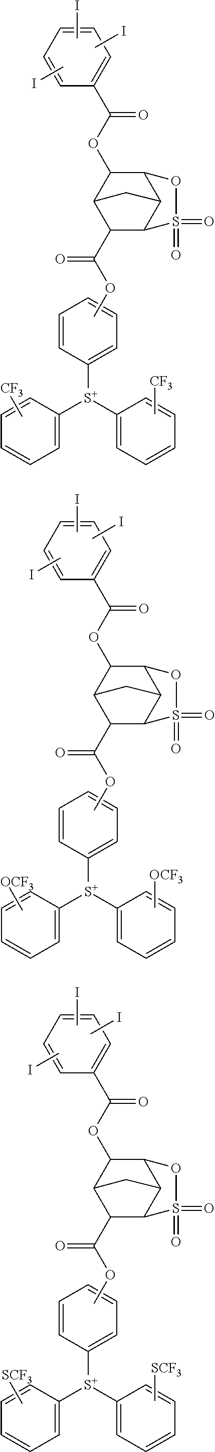

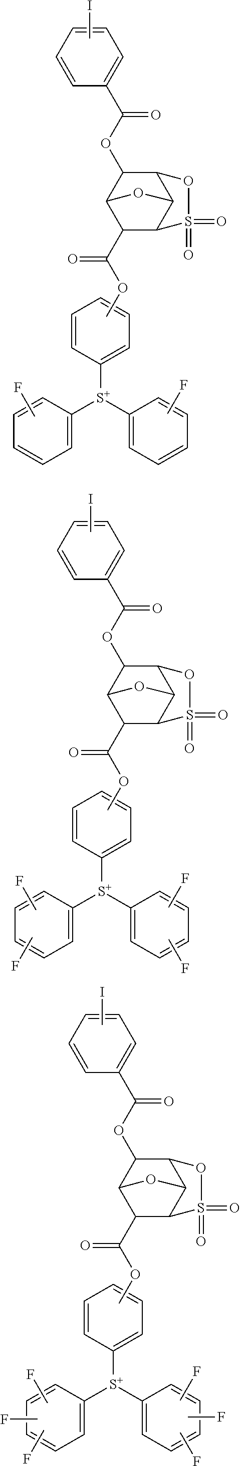

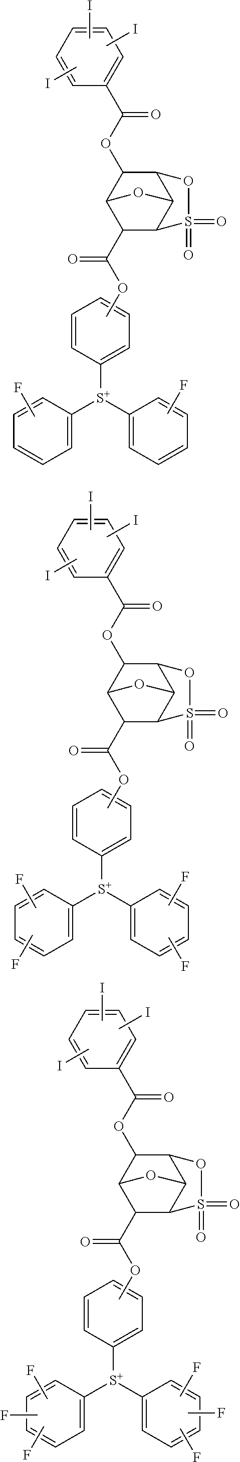

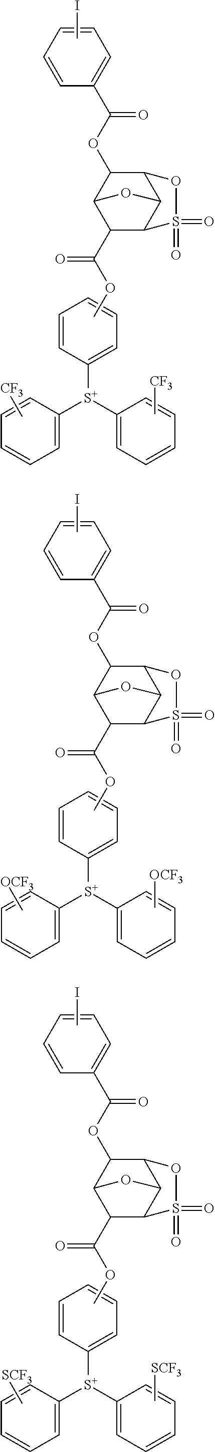

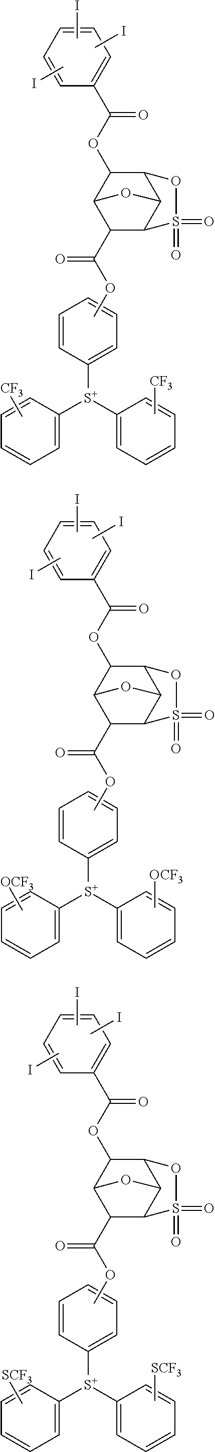

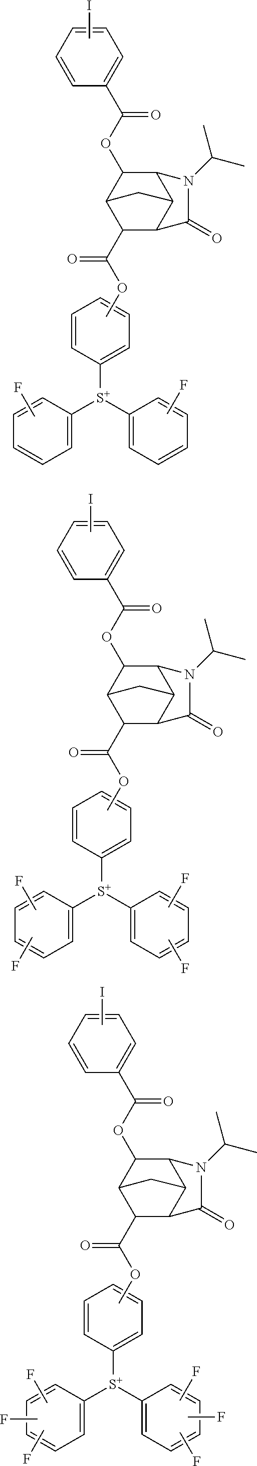

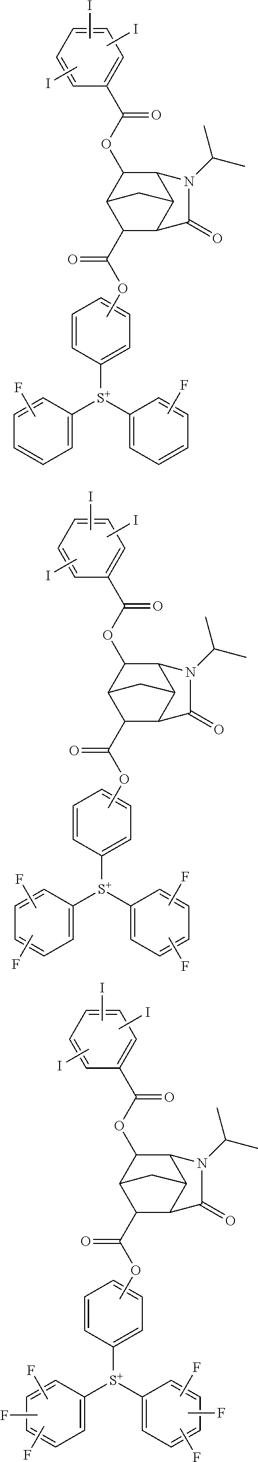

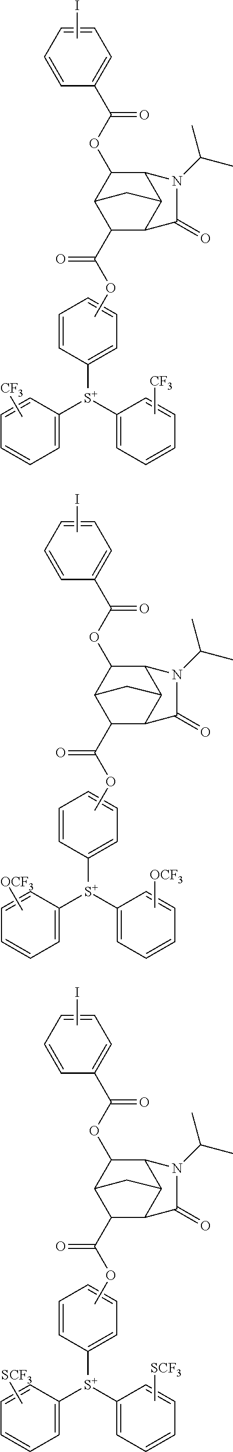

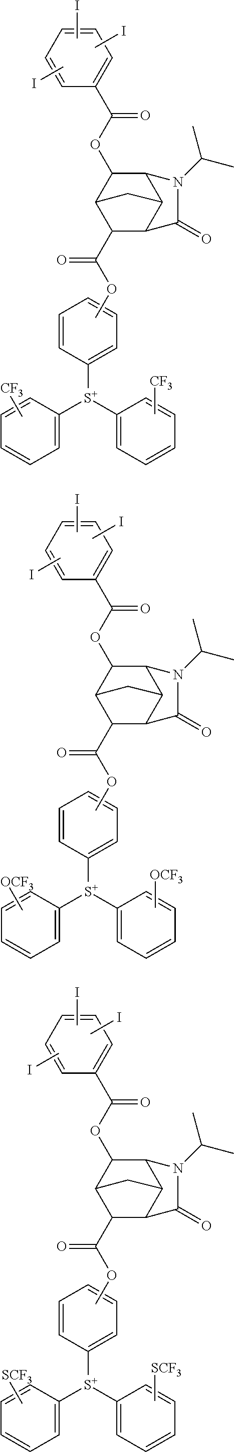

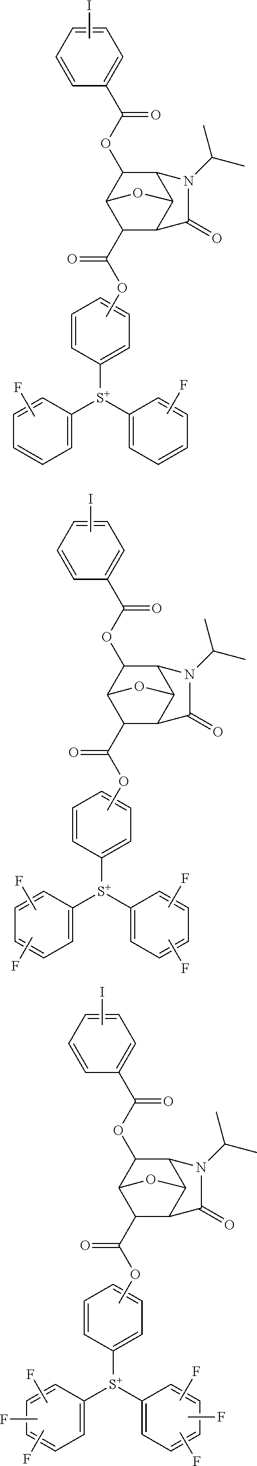

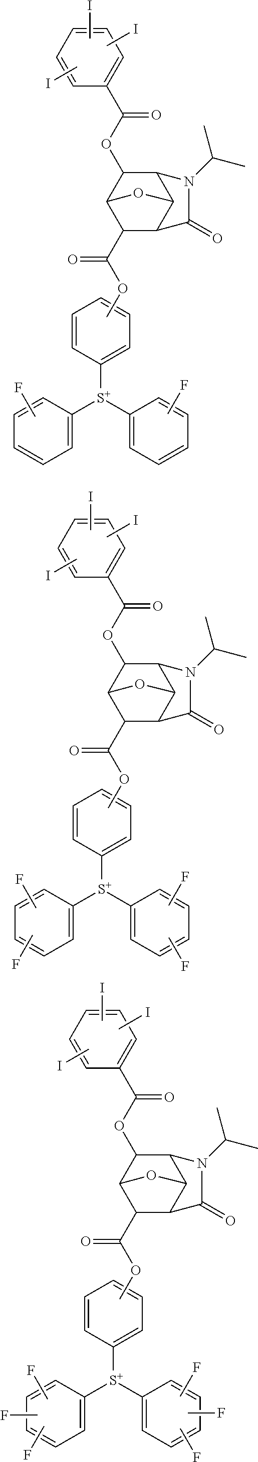

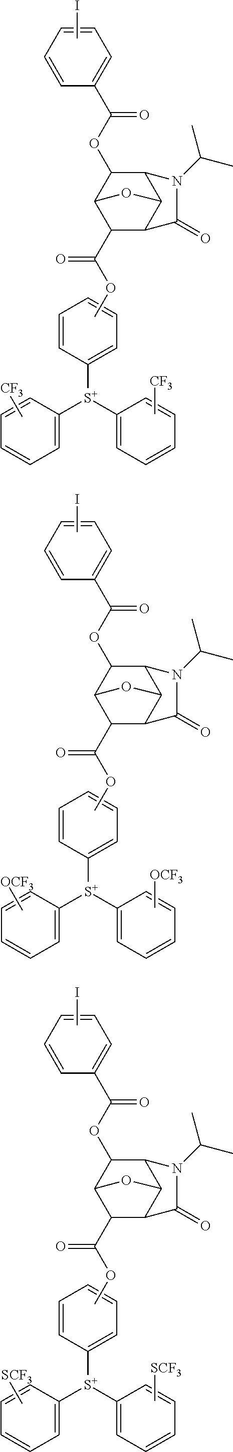

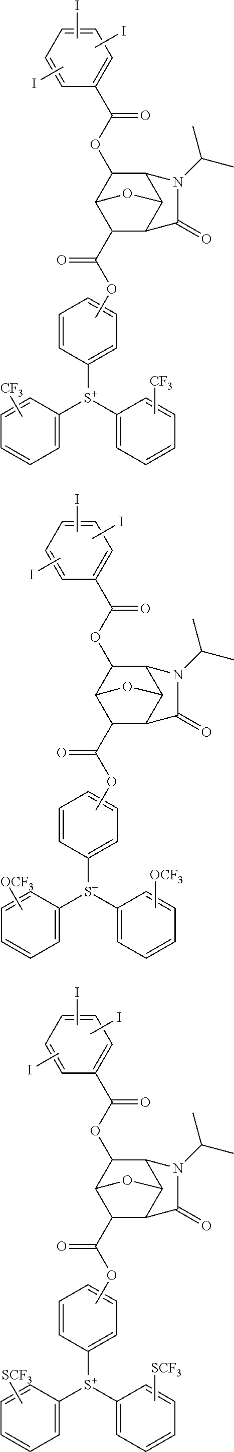

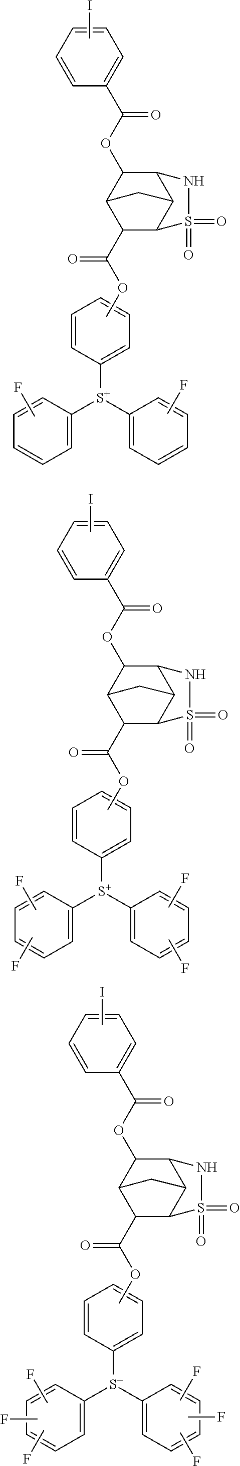

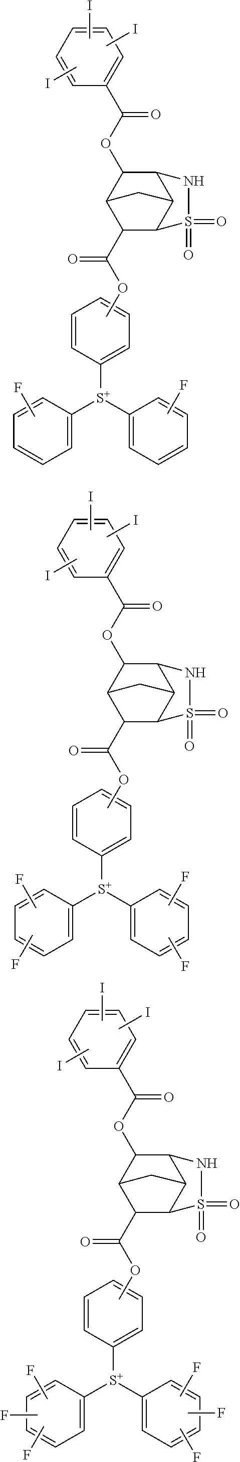

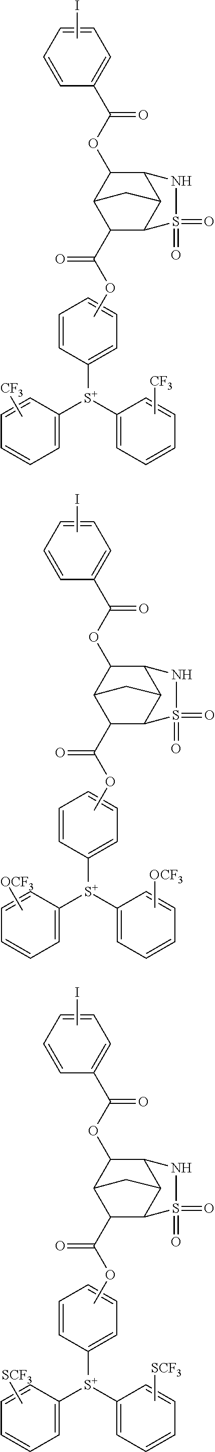

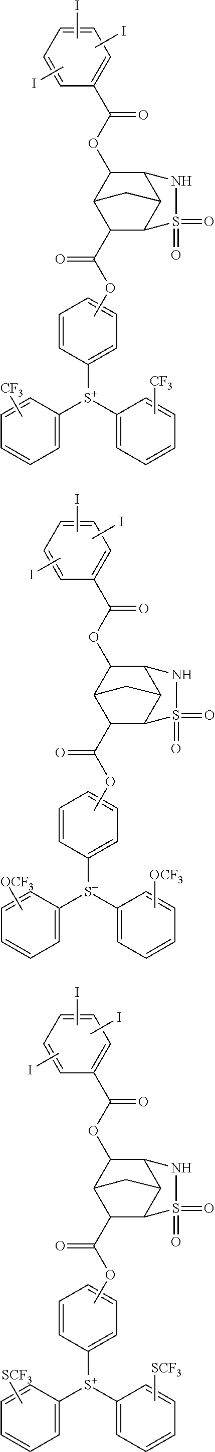

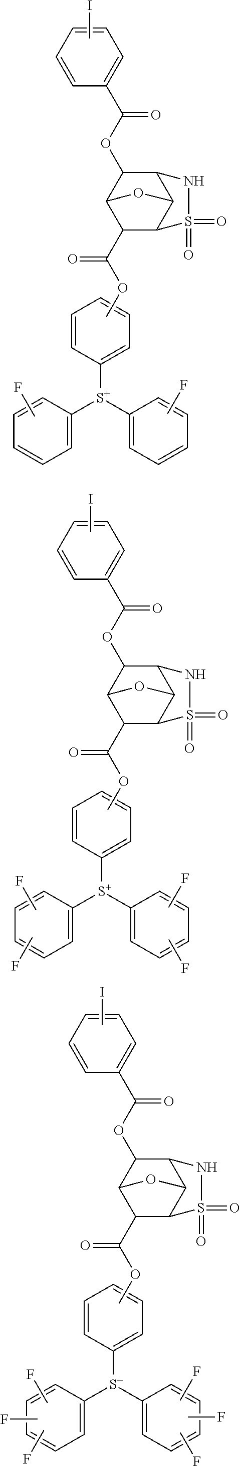

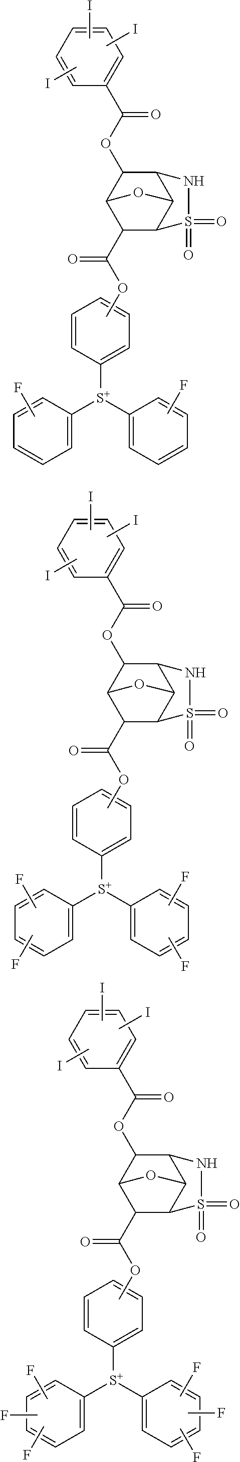

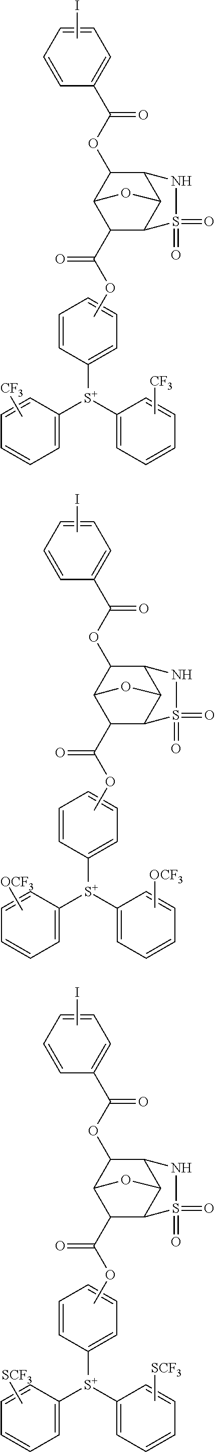

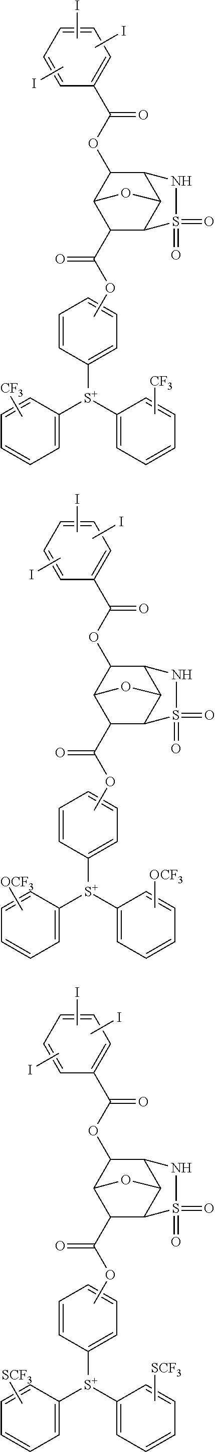

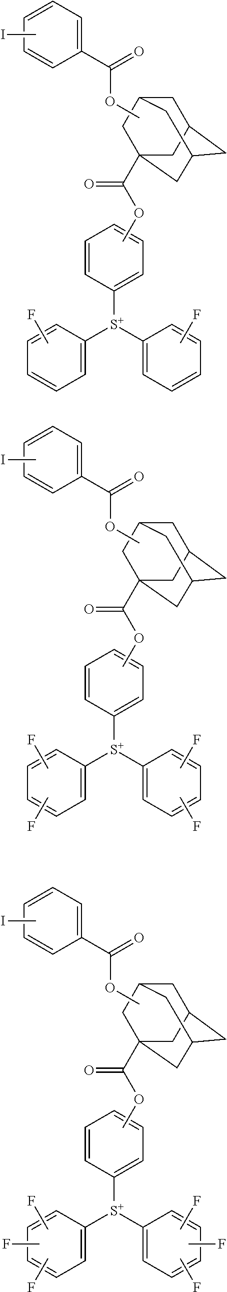

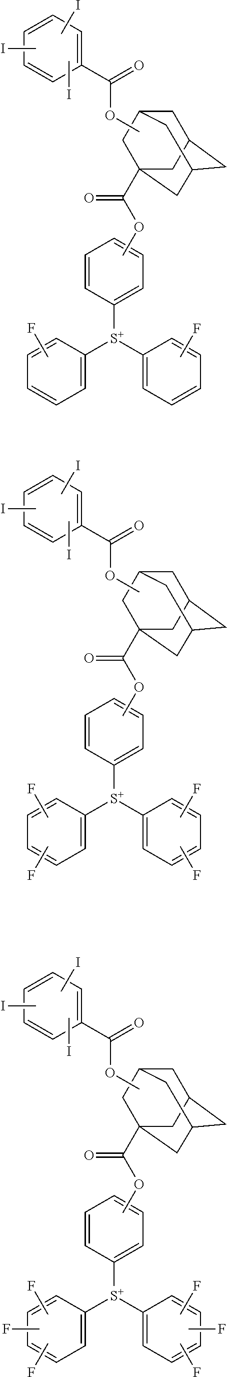

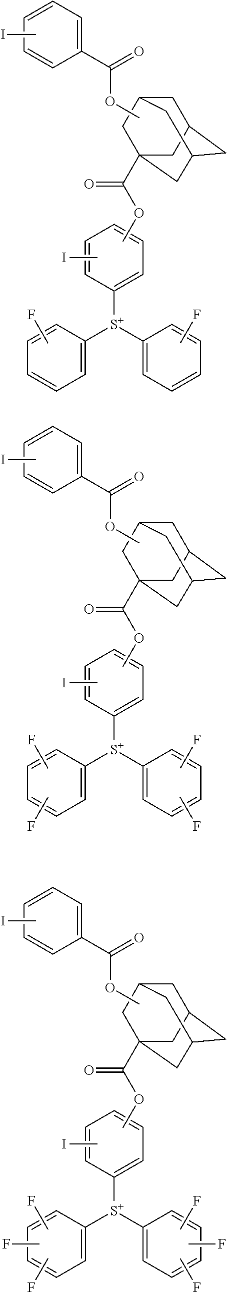

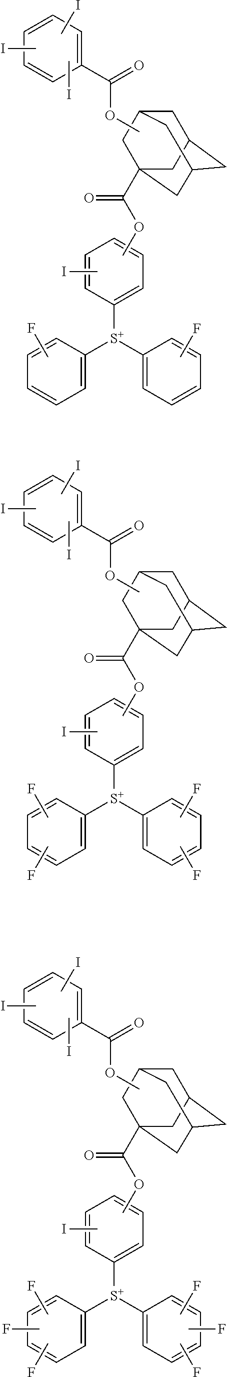

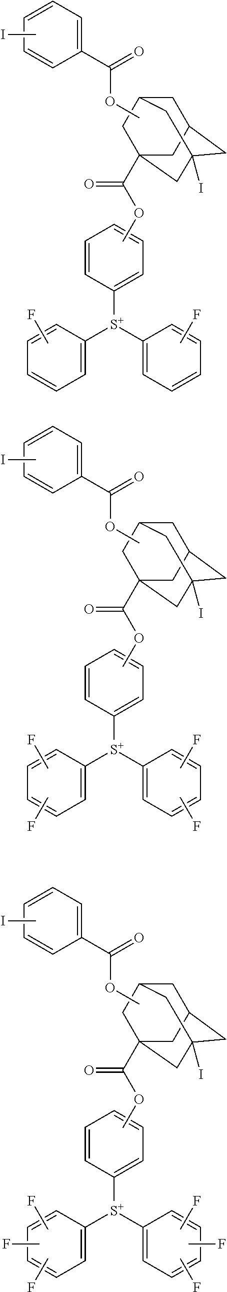

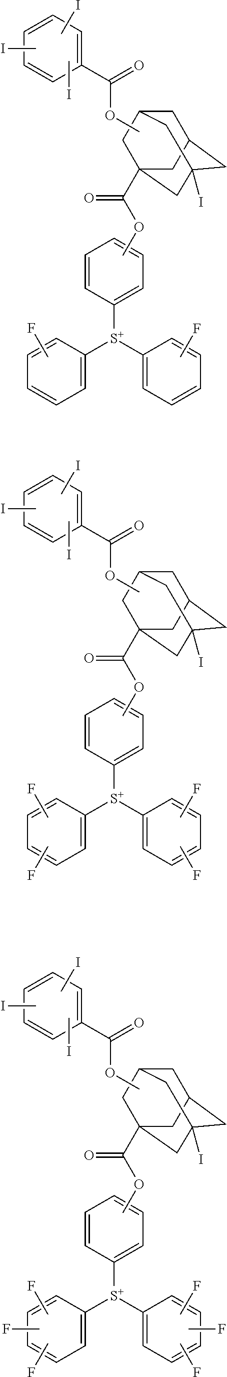

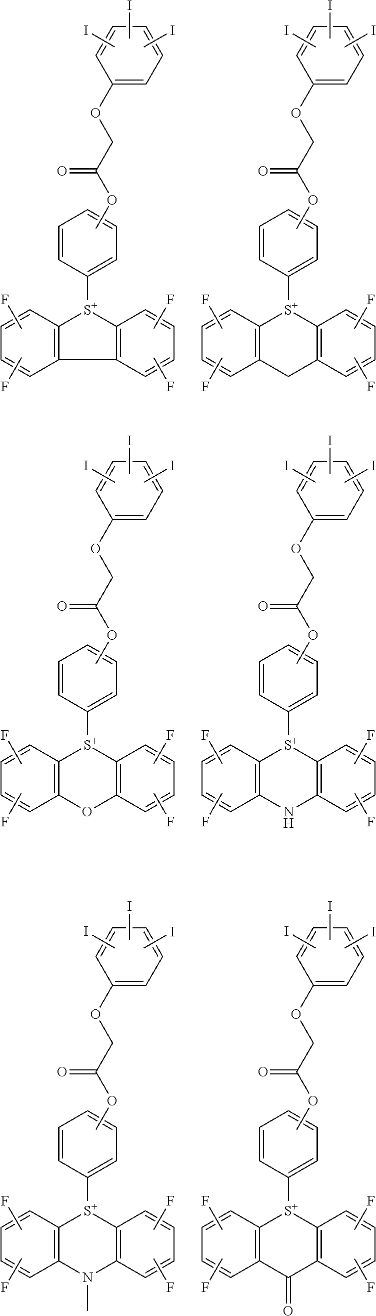

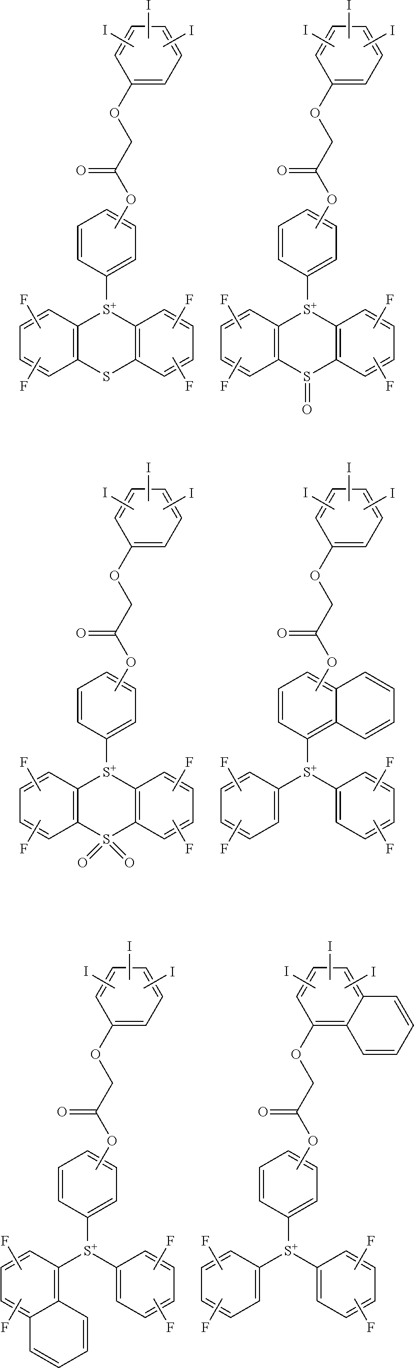

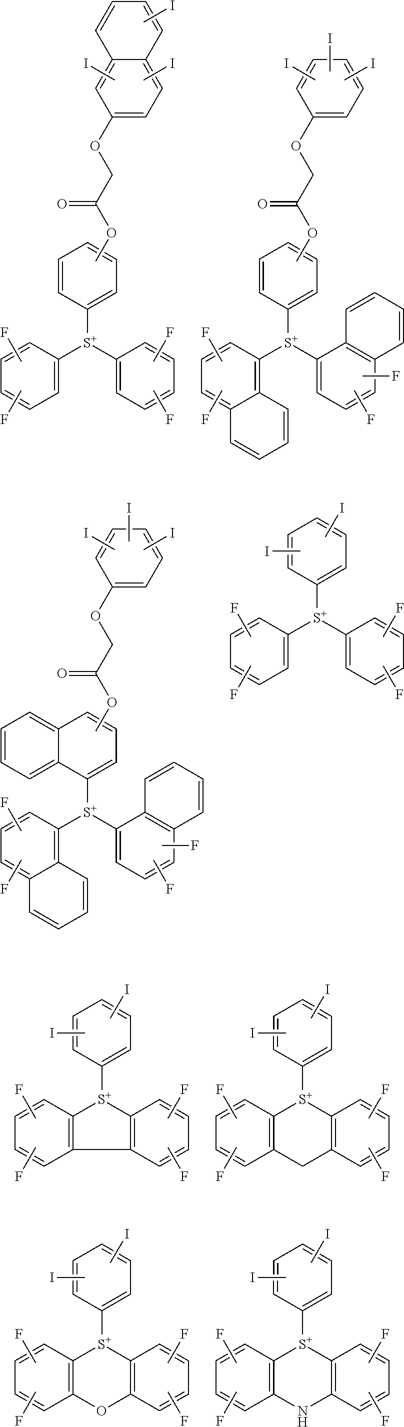

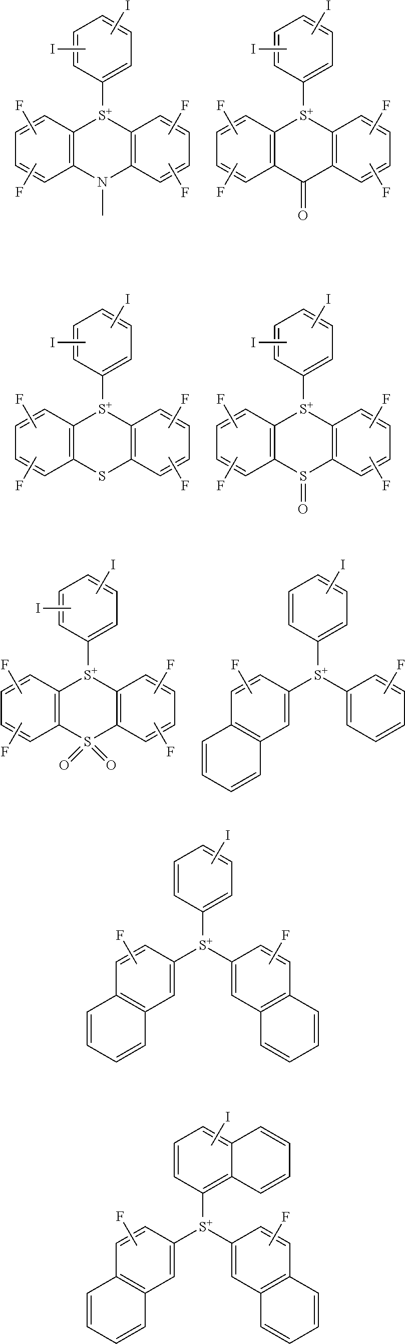

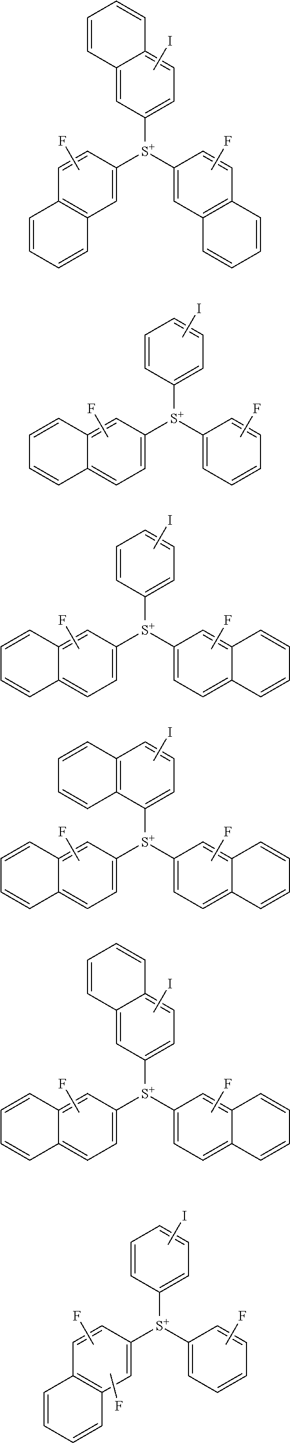

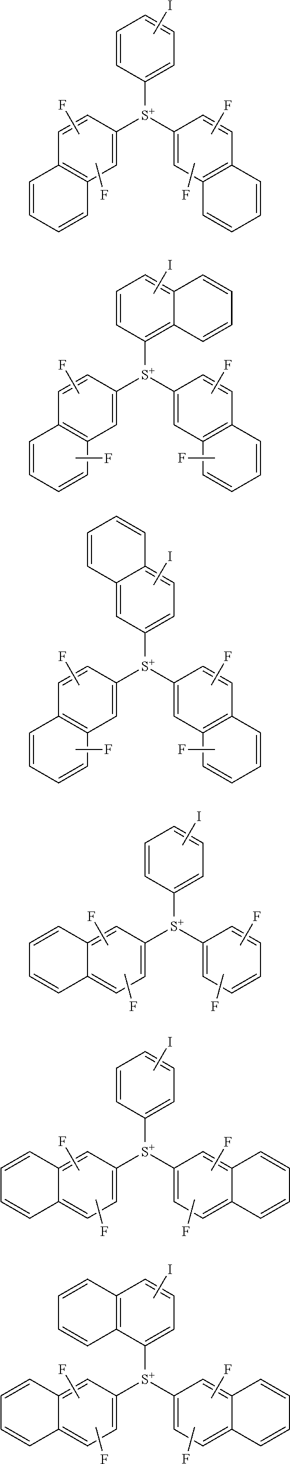

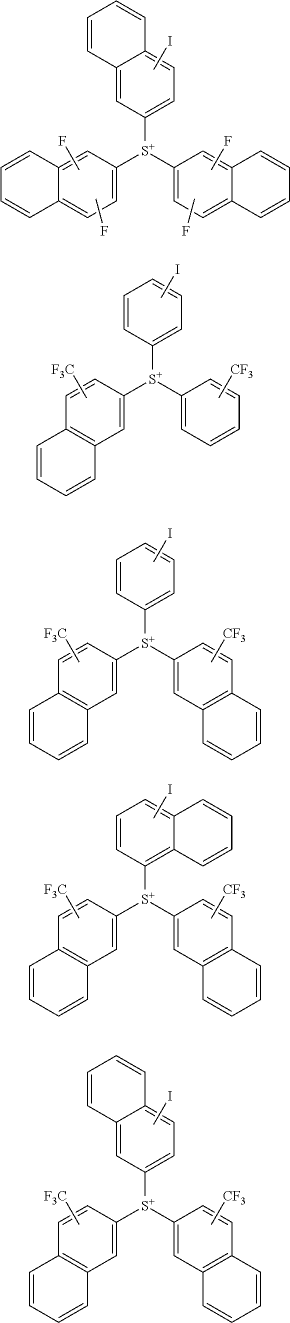

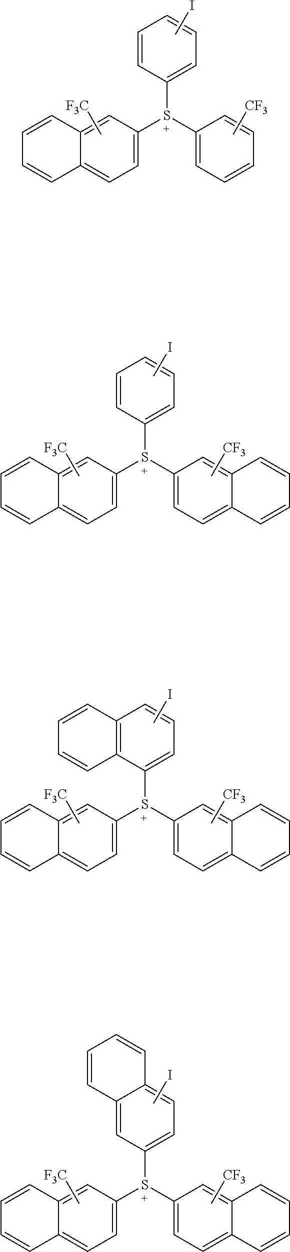

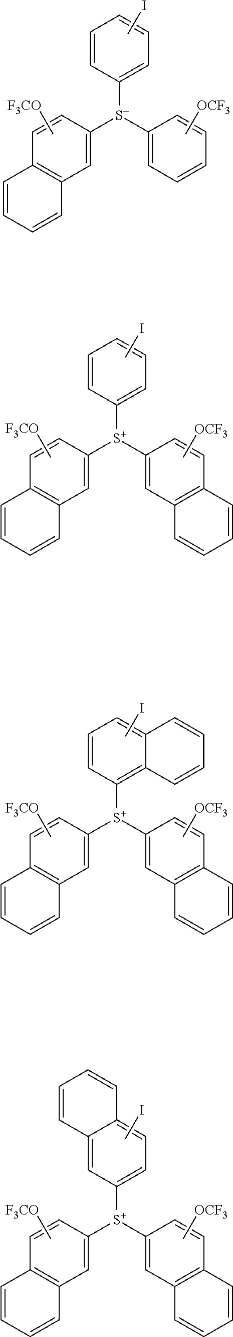

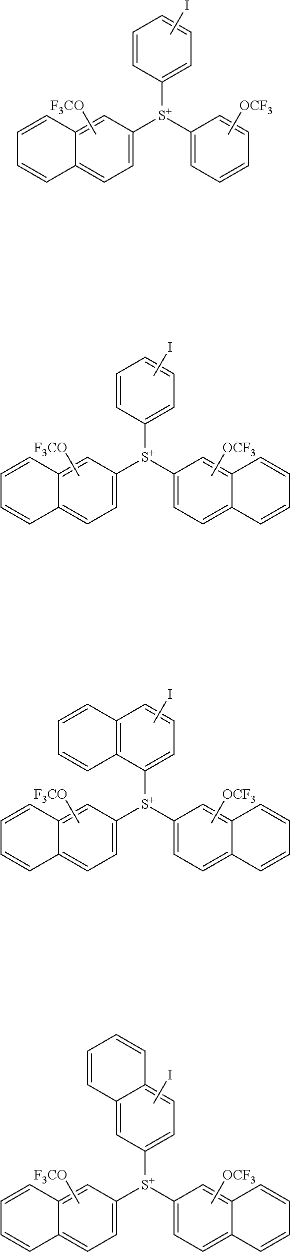

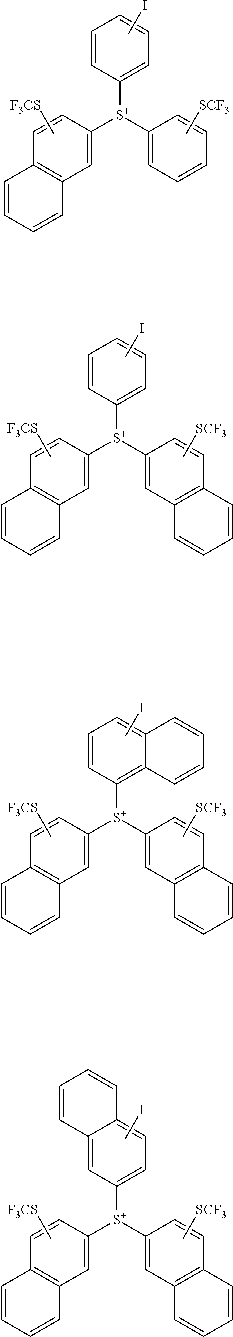

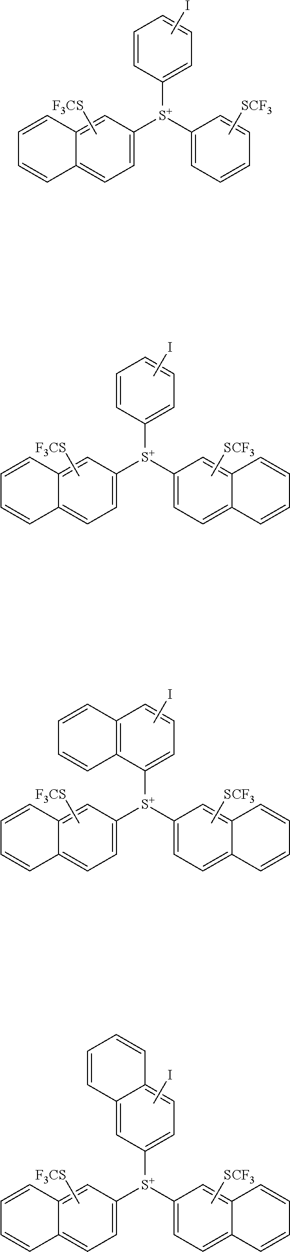

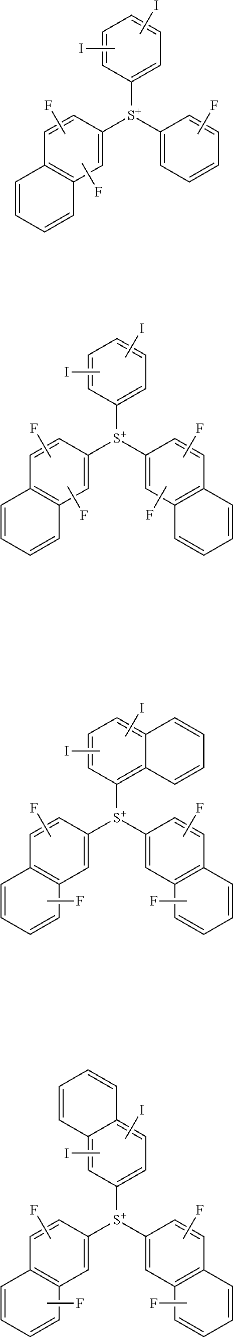

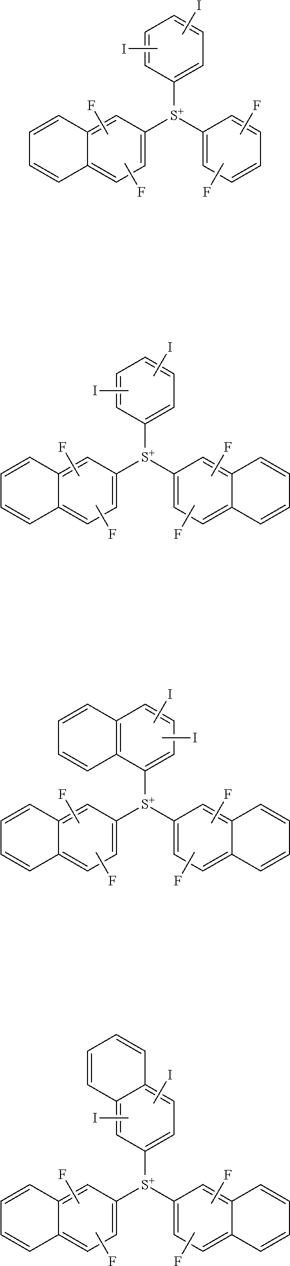

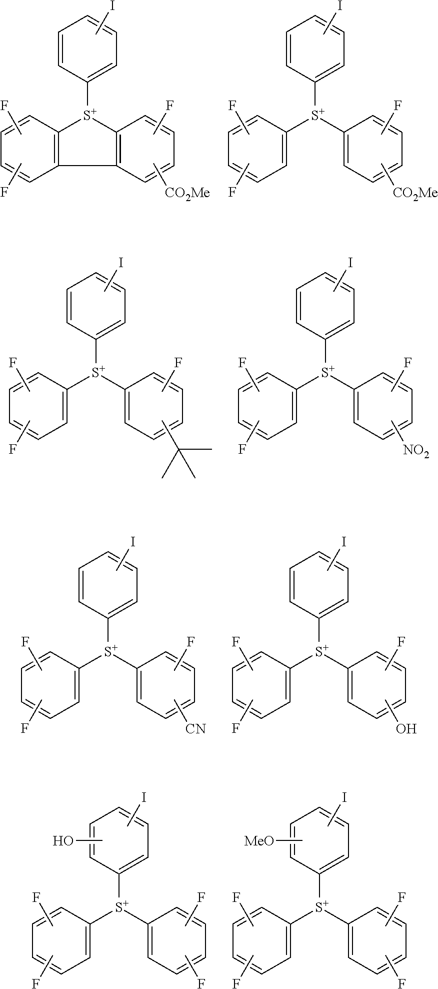

Examples of the iodonium cation include those described in JP-A 2024-000259, paragraph [0181], but not limited thereto.

Examples of the ammonium cation include ammonium cations having the formula (am-1).

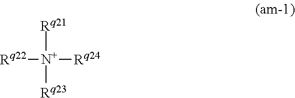

In the formula (am-1), R^{q21} to R^{q24} are each independently a C₁-C₄₀ hydrocarbyl group which may contain a heteroatom. R^{q21} and R^{q22} may bond together to form a ring with the nitrogen atoms to which they are attached. Examples of the hydrocarbyl group are as exemplified above for the hydrocarbyl group represented by R¹ in the formula (A).

Examples of the ammonium cations having the formula (am-1) include those shown below, but not limited thereto.

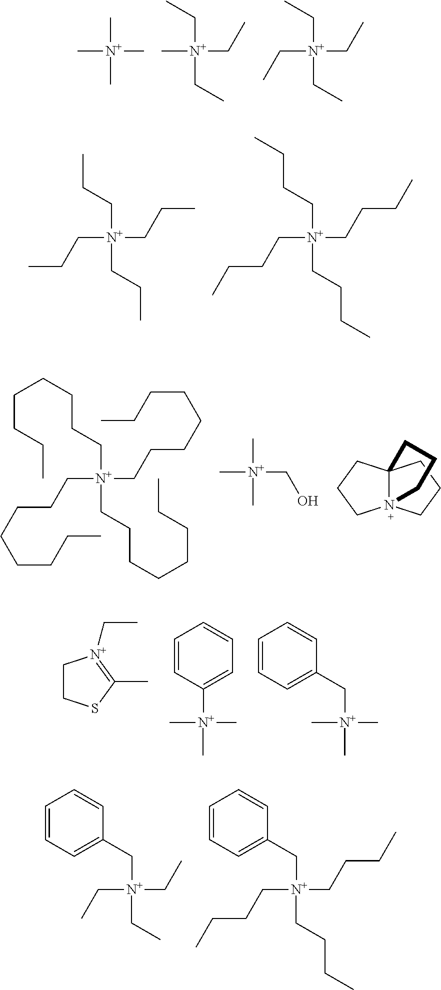

Examples of the onium salt having the formula (1) or (2) include arbitrary combinations of anions with cations, both as exemplified above. These onium salts may be readily prepared by ion exchange reaction using any well-known organic chemistry technique. For the ion exchange reaction, reference may be made to JP-A 2007-145797, for example.

The onium salt having the formula (1) or (2) functions as a quencher in the chemically amplified resist composition of the invention because the counter anion of the onium salt is a conjugated base of a weak acid. As used herein, the weak acid indicates an acidity insufficient to deprotect an acid labile group from an acid labile group-containing unit in the base polymer. The onium salt having the formula (1) or (2) functions as a quencher when used in combination with an onium salt type photoacid generator having a conjugate base of a strong acid (e.g., α-fluorinated sulfonic acid) as the counter anion. In a system using a mixture of an onium salt capable of generating a strong acid (e.g., α-fluorinated sulfonic acid) and an onium salt capable of generating a weak acid (e.g., non-fluorinated sulfonic acid or carboxylic acid), if the strong acid generated from the photoacid generator upon exposure to high-energy radiation collides with the unreacted onium salt having a weak acid anion, then a salt exchange occurs whereby the weak acid is released and an onium salt having a strong acid anion is formed. In this course, the strong acid is exchanged into the weak acid having a lower catalysis, incurring apparent deactivation of the acid for enabling to control acid diffusion.

Also, as the quencher (C), onium salts having sulfonium cation and phenoxide anion sites in a common molecule as described in JP 6848776, onium salts having sulfonium cation and carboxylate anion sites in a common molecule as described in JP 6583136 and JP-A 2020-200311, and onium salts having iodonium cation and carboxylate anion sites in a common molecule as described in JP 6274755 can also be used.

If a photoacid generator capable of generating a strong acid is an onium salt, an exchange from the strong acid generated upon exposure to high-energy radiation to a weak acid can take place as mentioned above, but it rarely happens that the weak acid generated upon exposure to high-energy radiation collides with the unreacted onium salt capable of generating a strong acid to induce a salt exchange. This is because of the phenomenon that an onium cation is more likely to form an ion pair with a stronger acid anion.

When the chemically amplified resist composition of the invention comprises the onium salt having the formula (1) or (2) as the quencher (C), the amount of the onium salt is preferably 0.1 to 20 parts by weight, more preferably 0.1 to 10 parts by weight per 80 parts by weight of the base polymer (A). As long as the amount of onium salt type quencher (C) is in the range, a satisfactory resolution is available without a substantial lowering of sensitivity. The onium salt having the formula (1) or (2) can be used alone or in combination of two or more.

The chemically amplified resist composition of the invention may also comprise a nitrogen-containing compound as the quencher (C). Examples of the nitrogen-containing compound of the component (C) include primary, secondary and tertiary amine compounds, specifically amine compounds having a hydroxy group, ether bond, ester bond, lactone ring, cyano group or sulfonate ester bond, as described in JP-A 2008-111103, paragraphs [0146]-[0164] (U.S. Pat. No. 7,537,880), and primary or secondary amine compounds protected with a carbamate group, as described in JP 3790649.

A sulfonic acid sulfonium salt having a nitrogen-containing substituent may also be used as the nitrogen-containing compound. This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and JP-A 2012-046501, for example.

When the chemically amplified resist composition of the invention comprises the nitrogen-containing compound as the quencher (C), the amount of the nitrogen-containing compound is preferably 0.001 to 12 parts by weight, more preferably 0.01 to 8 parts by weight per 80 parts by weight of the base polymer (A). The nitrogen-containing compound may be used alone or in combination of two or more.

### (D) Photoacid Generator

The chemically amplified resist composition of the invention may comprise a photoacid generator as component (D). The photoacid generator is not particularly limited as long as it is a compound that generates an acid upon exposure to high-energy radiation. Preferred examples of the photoacid generator include those having the formula (3) or (4).

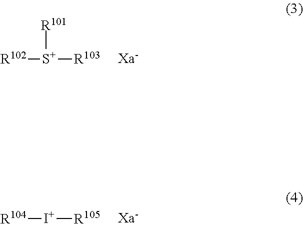

In the formula (3), R¹⁰¹ to R¹⁰⁵ are each independently halogen, or a C₁-C₂₀ hydrocarbyl group which may contain a heteroatom. Any two of R¹⁰¹, R 102 and R¹⁰³ may bond together to form a ring with the sulfur atoms to which they are attached.

Examples of the sulfonium cation in the salt having the formula (3) include those described as examples of the sulfonium cation in the formula (A), those described in JP-A 2024-003744, paragraphs to [0125], those described in WO 2024/128017, paragraphs to [0049], and those described in JP 7491173, paragraphs to [0046], and those described as examples of the sulfonium cation having the formula (sulfo-1), but not limited thereto. Examples of the iodonium cation in the salt having the formula (4) include those described in JP-A 2024-000259, paragraph [0181], but not limited thereto.

In the formulae (3) and (4), Xa⁻ is an anion of strong acid. Examples of the anion of strong acid include anions having any of the formulae (Xa-1) to (Xa-4).

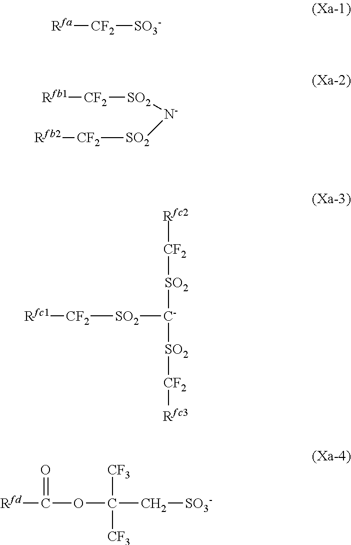

In the formula (Xa-1), R^{fa} is fluorine or a C₁-C₄₀ hydrocarbyl group which may contain a heteroatom. The hydrocarbyl group may be saturated or unsaturated and straight, branched or cyclic. Examples thereof include those exemplified above for the hydrocarbyl group represented by R^{fa1} in the formula (Xa-1-1).

As the anion having the formula (Xa-1), an anion having the formula (Xa-1-1) is preferred.

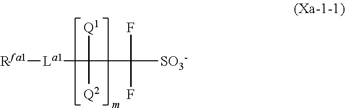

In the formula (Xa-1-1), Q¹ and Q² are each independently hydrogen, fluorine, or a C₁-C₆ fluorinated saturated hydrocarbyl group. It is preferred for solvent solubility that at least one of Q¹ and Q² be trifluoromethyl. m is 0, 1, 2, 3, or 4, most preferably 1. R^{fa1} is a C₁-C₃₅ hydrocarbyl group which may contain a heteroatom. As the heteroatom, oxygen, nitrogen, sulfur and halogen atoms are preferred, with oxygen being more preferred. Of the hydrocarbyl groups, those groups of 6 to 30 carbon atoms are preferred from the aspect of achieving a high resolution in forming small-size patterns.

In (Xa-1-1), the C₁-C₃₅ hydrocarbyl group represented by R^{fa1} may be saturated or unsaturated and straight, branched or cyclic. Examples thereof include C₁-C₃₅ alkyl groups such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl, and icosyl; C₃-C₃₅ cyclic saturated hydrocarbyl groups such as cyclopentyl, cyclohexyl, 1-adamantyl, 2-adamantyl, 1-adamantylmethyl, norbornyl, norbornylmethyl, tricyclodecyl, tetracyclododecyl, tetracyclododecylmethyl, and dicyclohexylmethyl; C₂-C₃₅ unsaturated aliphatic hydrocarbyl groups such as 2-propenyl and 3-cyclohexenyl; C₆-C₃₅ aryl groups such as phenyl, 1-naphthyl, 2-naphthyl, and 9-fluorenyl; and C₇-C₃₅ aralkyl groups such as benzyl and diphenylmethyl; and combinations thereof.

In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, nitro moiety, carbonyl moiety, ether bond, ester bond, sulfonate ester bond, carbonate bond, lactone ring, sultone ring, carboxylic anhydride (—C(═O)—O—C(═O)—) or haloalkyl moiety. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl and 3-oxocyclohexyl groups.

In the formula (Xa-1-1), L^{a1} is a single bond, ether bond, ester bond, sulfonate ester bond, carbonate bond or carbamate bond. From the aspect of synthesis, an ether bond or ester bond is preferred, with the ester bond being more preferred.

Examples of the anion having formula (Xa-1) include those shown below, but not limited thereto. In the formulae, Q¹ is as described above, and Ac is acetyl.

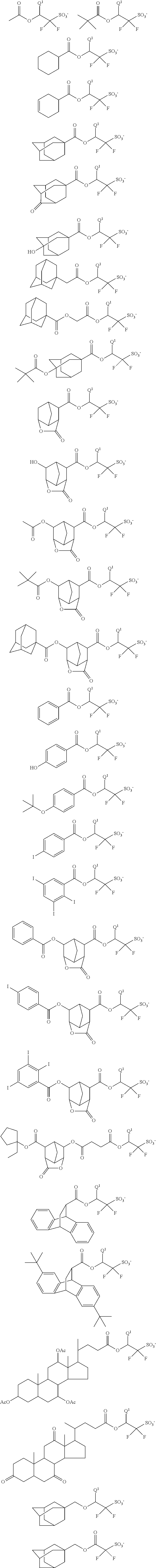

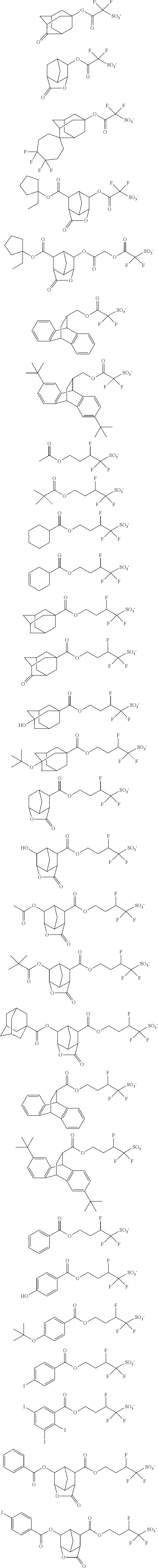

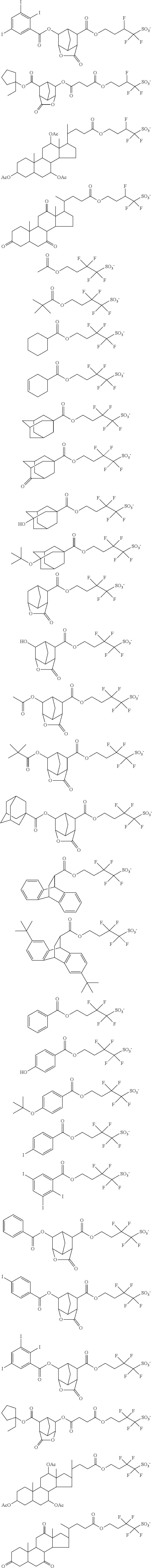

In the formula (Xa-2), R^{fb1} and R^{fb2} are each independently fluorine or a C₁-C₄₀ hydrocarbyl group which may contain a heteroatom. The hydrocarbyl group may be saturated or unsaturated and straight, branched or cyclic. Examples thereof include those exemplified above for the hydrocarbyl group represented by R^{fa1} in the formula (Xa-1-1). R^{fb1} and R^{fb2} are preferably fluorine or C₁-C₄ straight fluorinated alkyl groups. Also, R^{fb1} and R^{fb2} may bond together to form a ring with the linkage: —CF₂—SO₂—N⁻—SO₂—CF₂— to which they are attached. It is preferred that a combination of R^{fb1} and R^{fb2} be a fluorinated ethylene or fluorinated propylene group.

In the formula (Xa-3), R^{fc1}, R^{fc2} and R^{fc3} are each independently fluorine or a C₁-C₄₀ hydrocarbyl group which may contain a heteroatom. The hydrocarbyl group may be saturated or unsaturated and straight, branched or cyclic. Examples thereof include those exemplified above for the hydrocarbyl group represented by R^{fa1} in the formula (Xa-1-1). R^{fc1}, R^{fc2} and R^{fc3} are preferably fluorine or C₁-C₄ straight fluorinated alkyl groups. Also, R^{fc1} and R^{fc2} may bond together to form a ring with the linkage: —CF₂—SO₂—C⁻—SO₂—CF₂— to which they are attached. It is preferred that a combination of R^{fc1} and R^{fc2} be a fluorinated ethylene or fluorinated propylene group.

In the formula (Xa-4), R^{fd} is a C₁-C₄₀ hydrocarbyl group which may contain a heteroatom. The hydrocarbyl group may be saturated or unsaturated and straight, branched or cyclic. Examples thereof include those exemplified above for the hydrocarbyl group represented by R^{fa1} in the formula (Xa-1-1).

Examples of the anion having the formula (Xa-4) include those shown below, but not limited thereto.

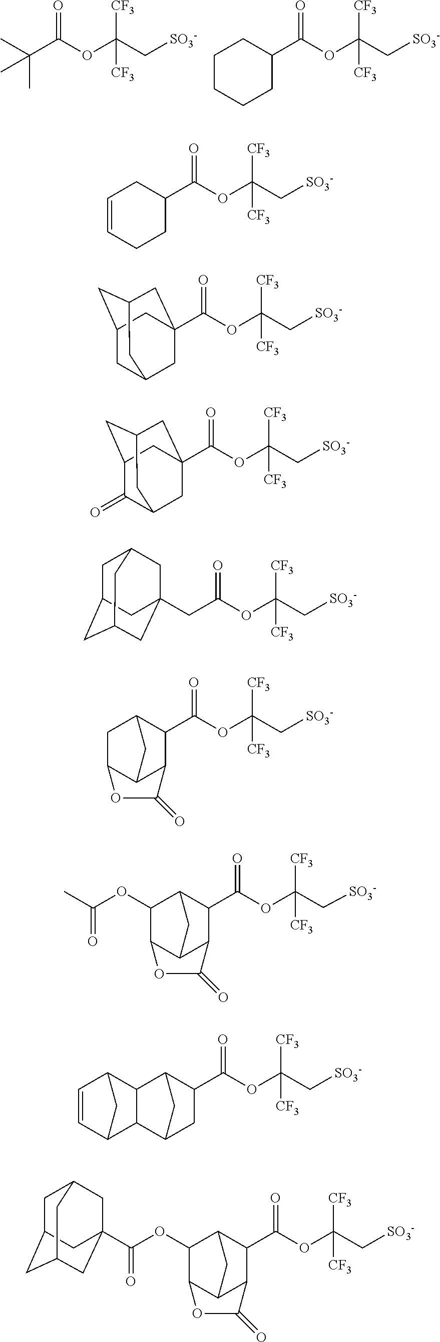

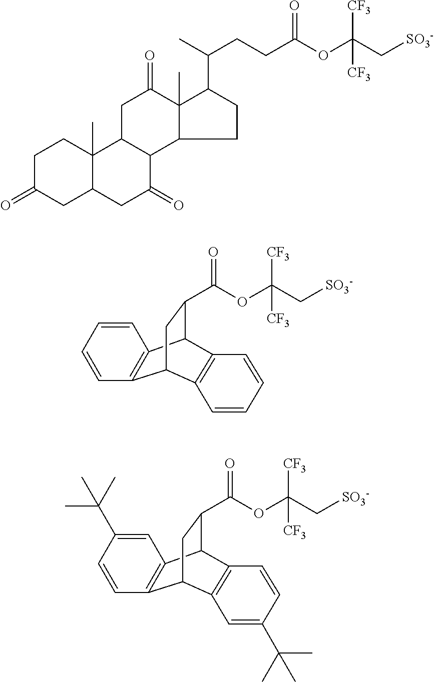

Examples of the non-nucleophilic counter ion further include anions having an aromatic ring substituted with iodine or bromine. Examples of the anions include anions having the formula (Xa-5).

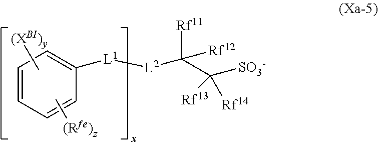

In the formula (Xa-5), x is 1, 2 or 3. y is 1, 2, 3, 4, or 5.

z is 0, 1, 2, or 3. It is noted that 1≤y+z≤5. y is preferably 1, 2, or 3, more preferably 2 or 3. z is preferably 0, 1, or 2.

In the formula (Xa-5), X^{B1} is iodine or bromine. A plurality of X^{B1} may be identical or different when x and/or y is 2 or more.

In the formula (Xa-5), L¹ is a single bond, ether bond, ester bond, or a C₁-C₆ saturated hydrocarbylene group which may contain an ether bond or ester bond. The saturated hydrocarbylene group may be straight, branched or cyclic.

In the formula (Xa-5), L² is a single bond or a C₁-C₂₀ divalent linking group when x=1, or a C₁-C₂₀ (x+1)-valent linking group when x=2 or 3. The linking group may contain an oxygen, sulfur or nitrogen atom.

In the formula (Xa-5), R^{fe} is hydroxy, carboxy, fluorine, chlorine, bromine, amino group, or a C₁-C₂₀ hydrocarbyl, C₁-C₂₀ hydrocarbyloxy, C₂-C₂₀ hydrocarbylcarbonyl, C₂-C₂₀ hydrocarbyloxycarbonyl, C₂-C₂₀ hydrocarbylcarbonyloxy, or C₁-C₂₀ hydrocarbylsulfonyloxy group, which may contain fluorine, chlorine, bromine, a hydroxy moiety, amino moiety or ether bond, or —N(R^{feA}) (R^{feB}), —N(R^{feC})—C(═O)—R^{feD} or —N(R^{feC})—C(═O)—O—R^{feD}. R^{feA} and R^{feB} are each independently hydrogen or a C₁-C₆ saturated hydrocarbyl group. R^{feC} is hydrogen, or a C₁-C₆ saturated hydrocarbyl group which may contain halogen, a hydroxy, C₁-C₆ saturated hydrocarbyloxy, C₂-C₆ saturated hydrocarbylcarbonyl or C₂-C₆ saturated hydrocarbylcarbonyloxy moiety. R^{feD} is a C₁-C₁₆ aliphatic hydrocarbyl group, C₆-C₁₂ aryl group or C₇-C₁₅ aralkyl group, which may contain halogen, a hydroxy, C₁-C₆ saturated hydrocarbyloxy, C₂-C₆ saturated hydrocarbylcarbonyl or C₂-C₆ saturated hydrocarbylcarbonyloxy moiety. The aliphatic hydrocarbyl group may be saturated or unsaturated and straight, branched or cyclic. The hydrocarbyl, hydrocarbyloxy, hydrocarbylcarbonyl, hydrocarbyloxycarbonyl, hydrocarbylcarbonyloxy, and hydrocarbylsulfonyloxy groups may be straight, branched or cyclic. A plurality of R^{fe} may be identical or different when x and/or z is 2 or more.

Of these, R^{fe} is preferably hydroxy, —N(R^{feC})—C(═O)—R^{feD}, —N(R^{feC})—C(═O)—O—R^{feD}, fluorine, chlorine, bromine, methyl or methoxy.

In the formula (Xa-5), Rf¹¹ to Rf¹⁴ are each independently hydrogen, fluorine or trifluoromethyl, and at least one of Rf¹¹ to Rf¹⁴ is fluorine or trifluoromethyl. Rf¹¹ and Rf¹², taken together, may form a carbonyl group. More preferably, both Rf¹³ and Rf¹⁴ are fluorine.

Examples of the anion having the formula (Xa-5) include those shown below, but not limited thereto. In the formulae. X^{B1} is as defined above.

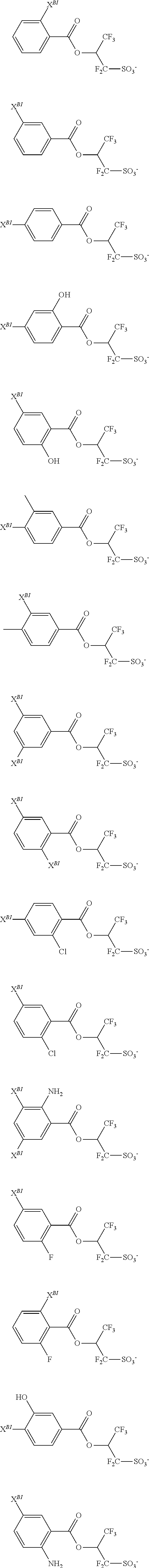

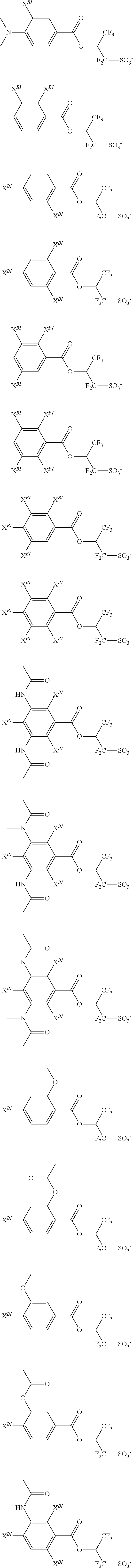

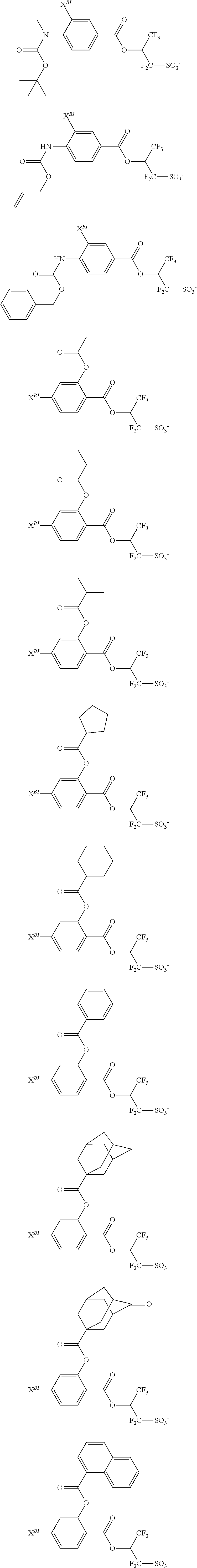

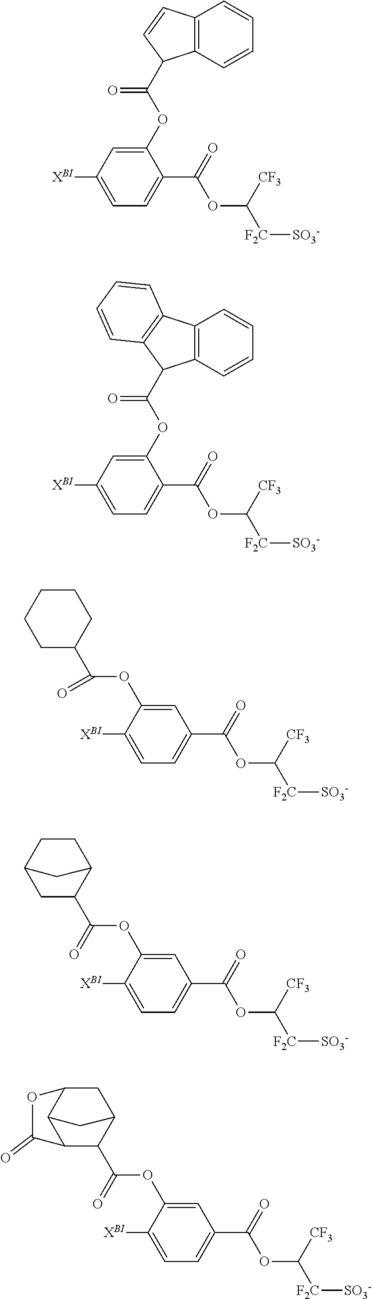

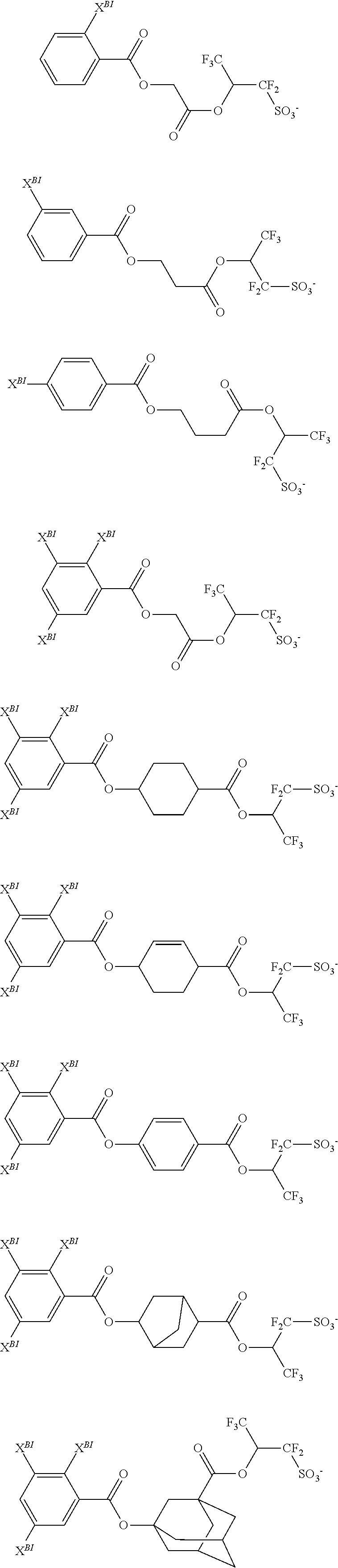

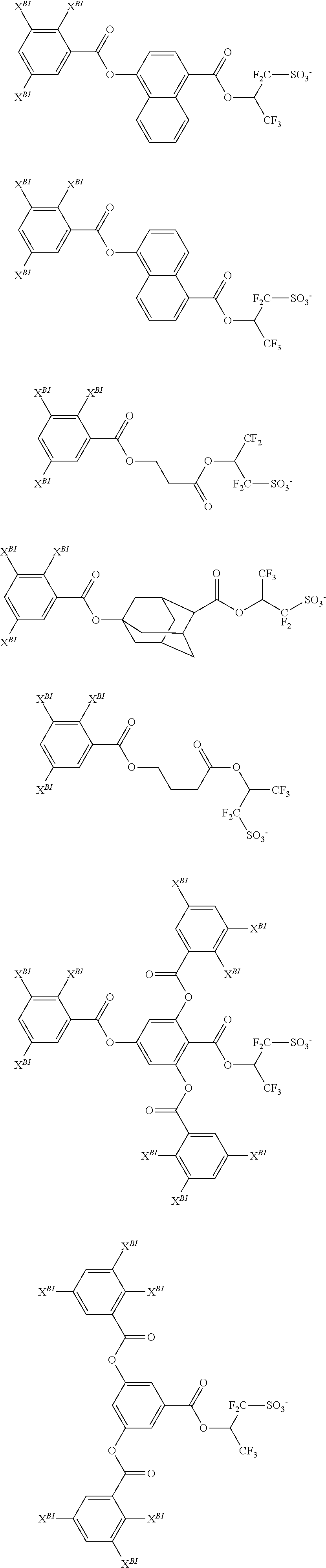

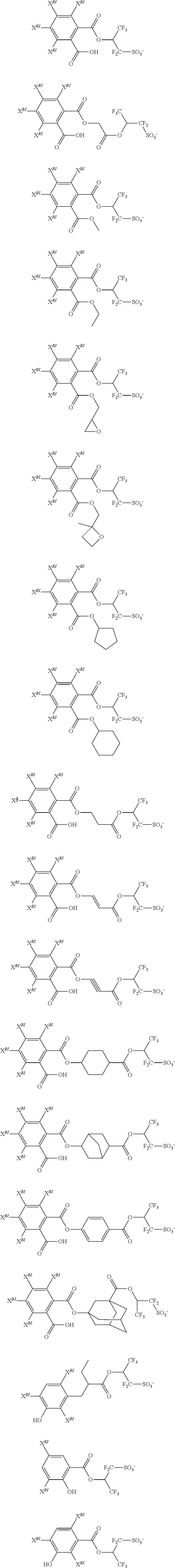

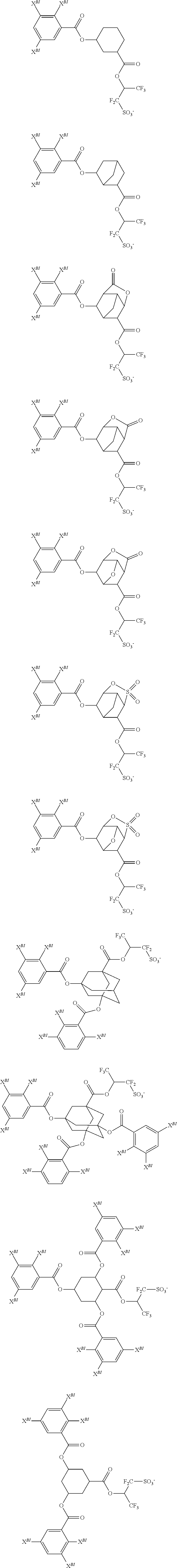

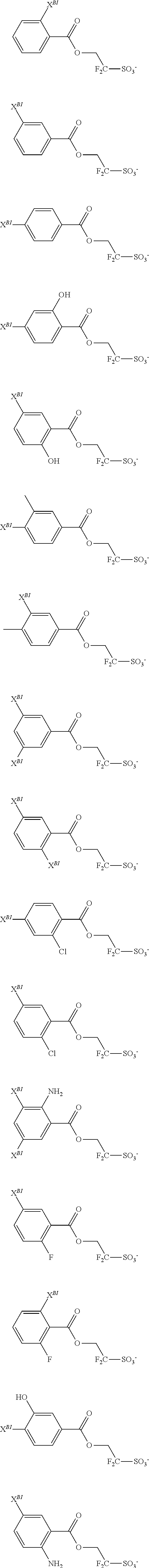

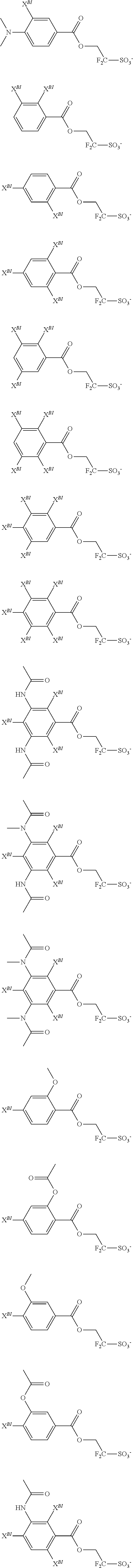

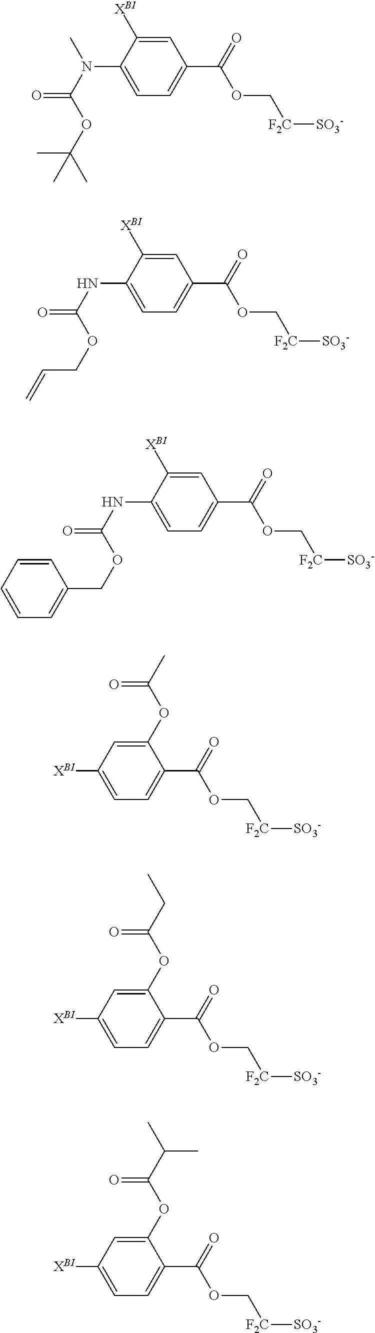

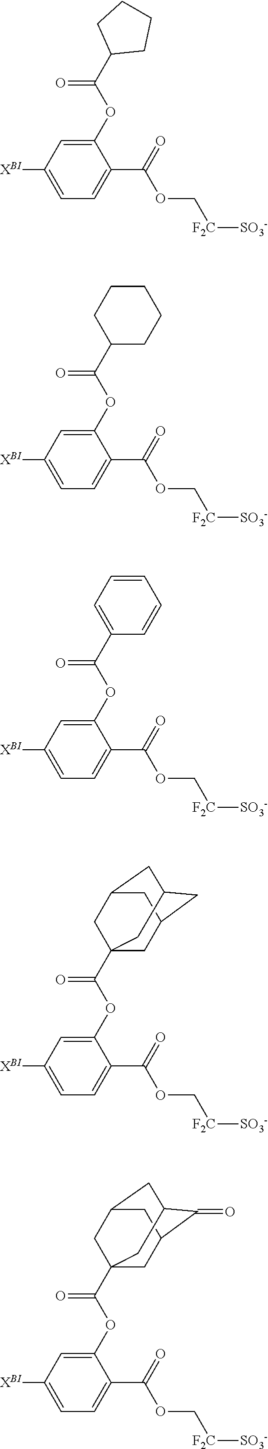

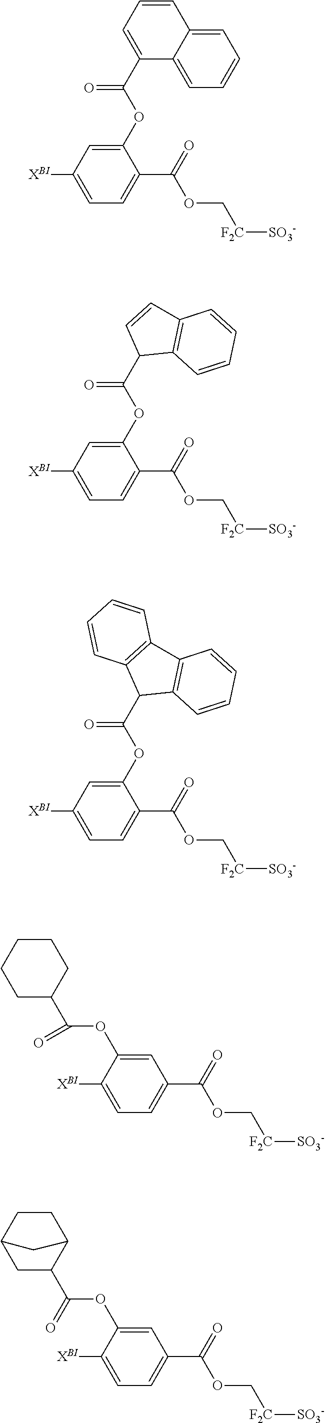

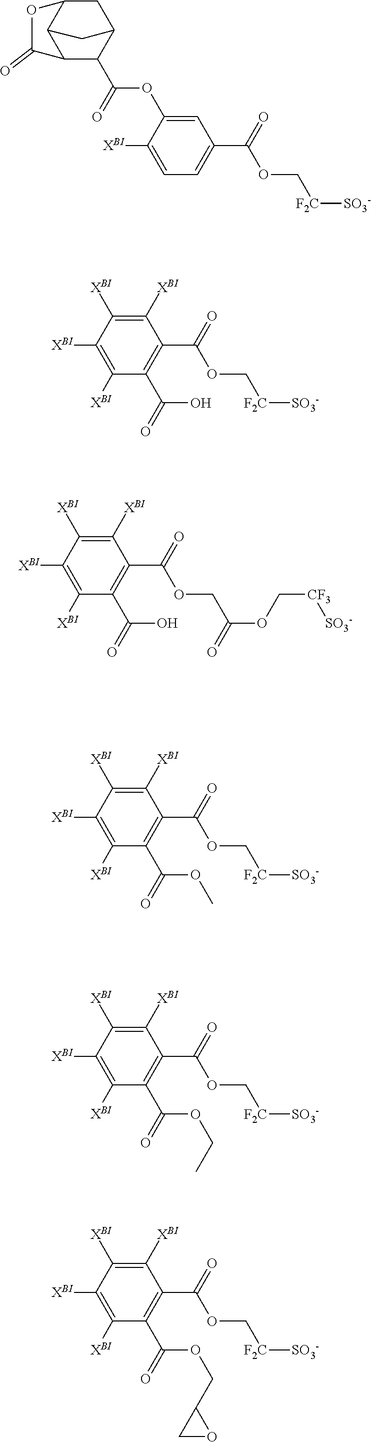

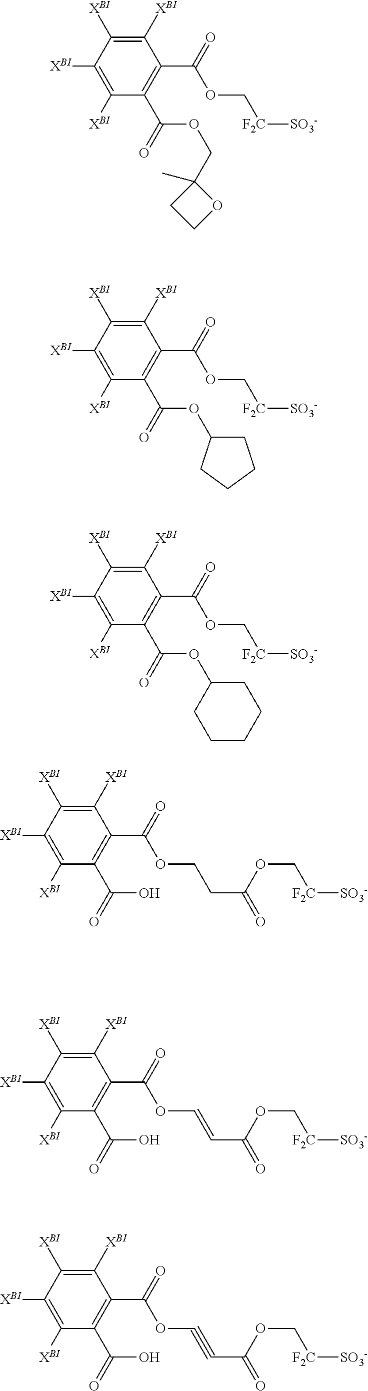

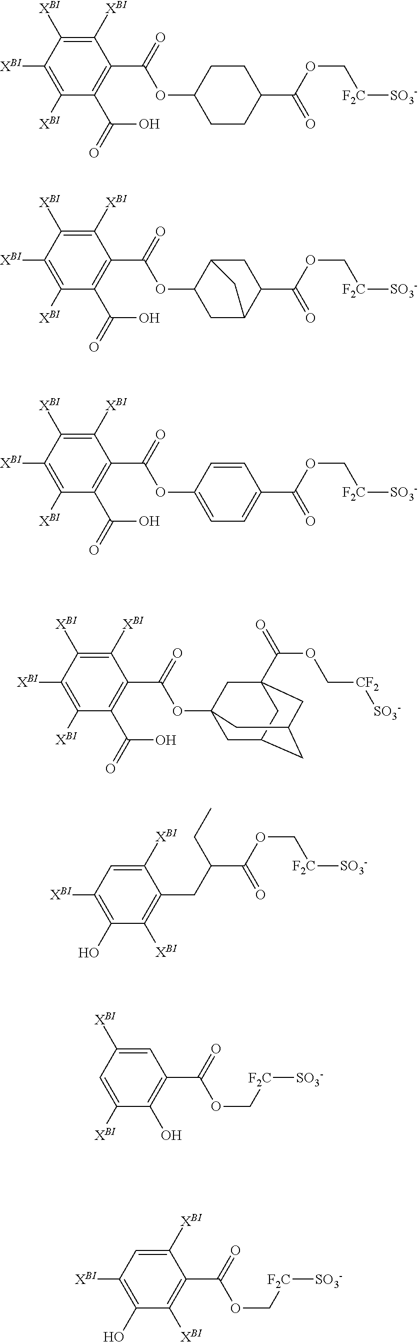

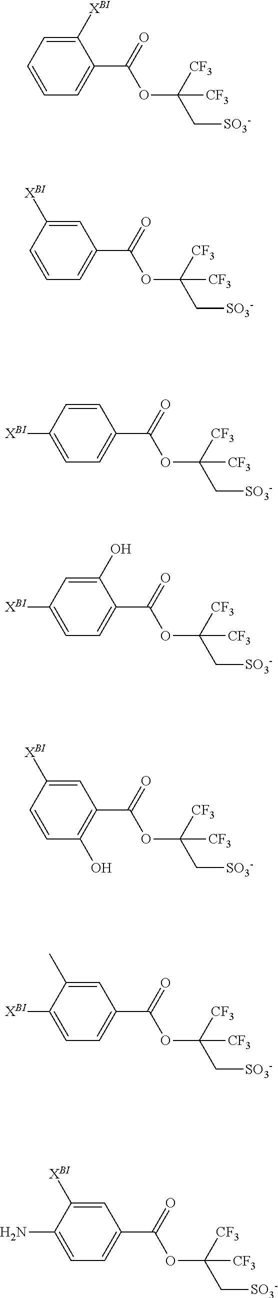

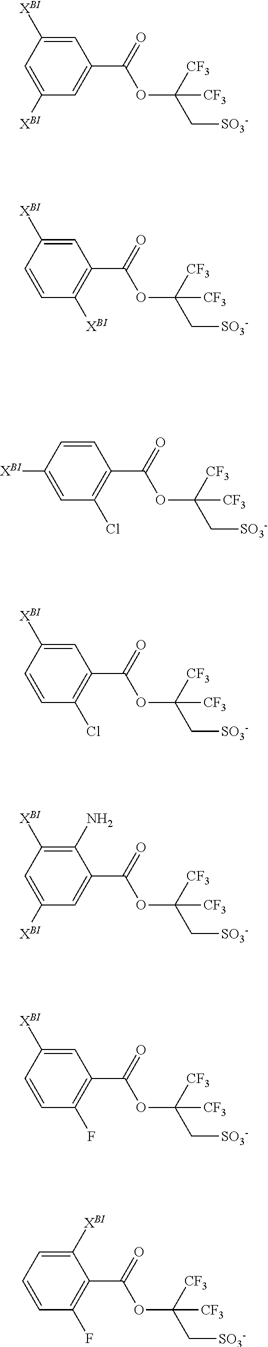

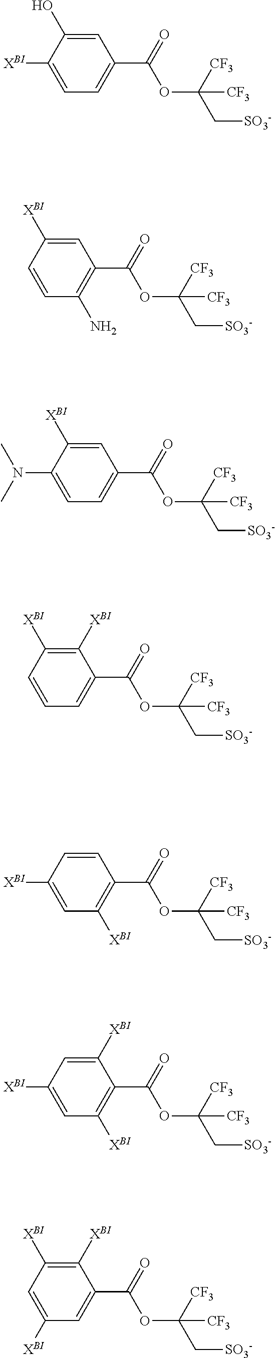

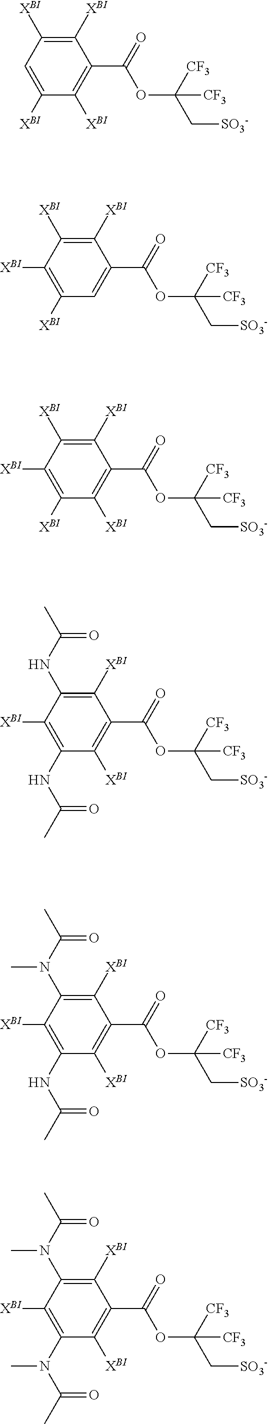

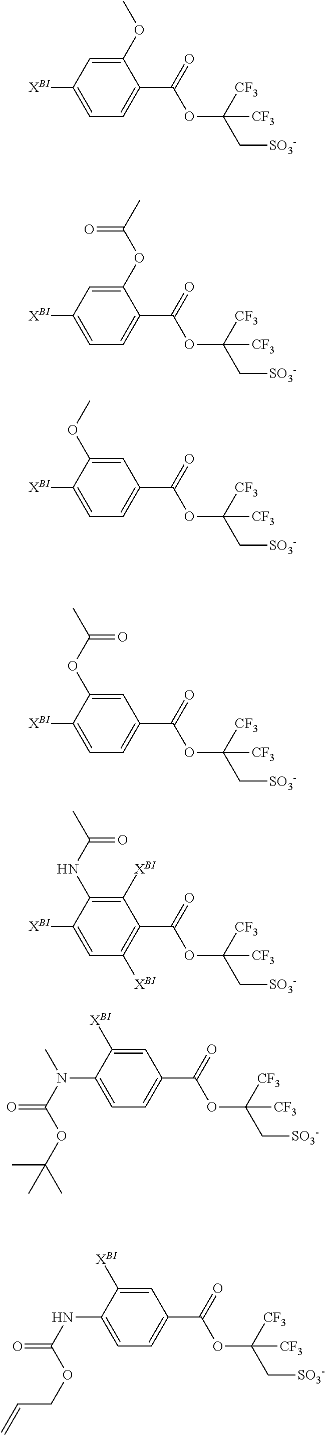

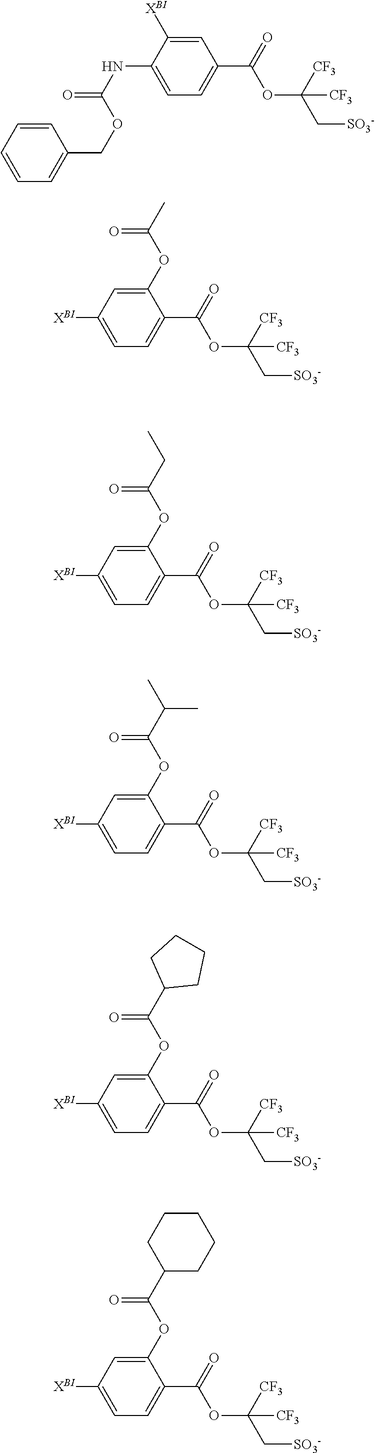

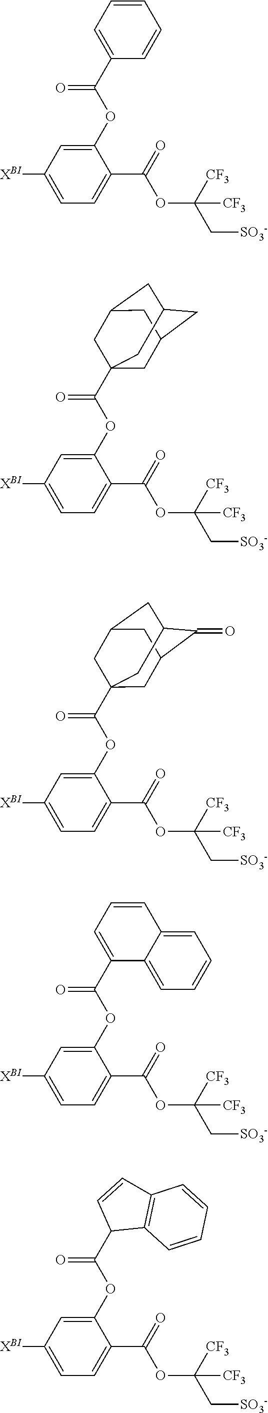

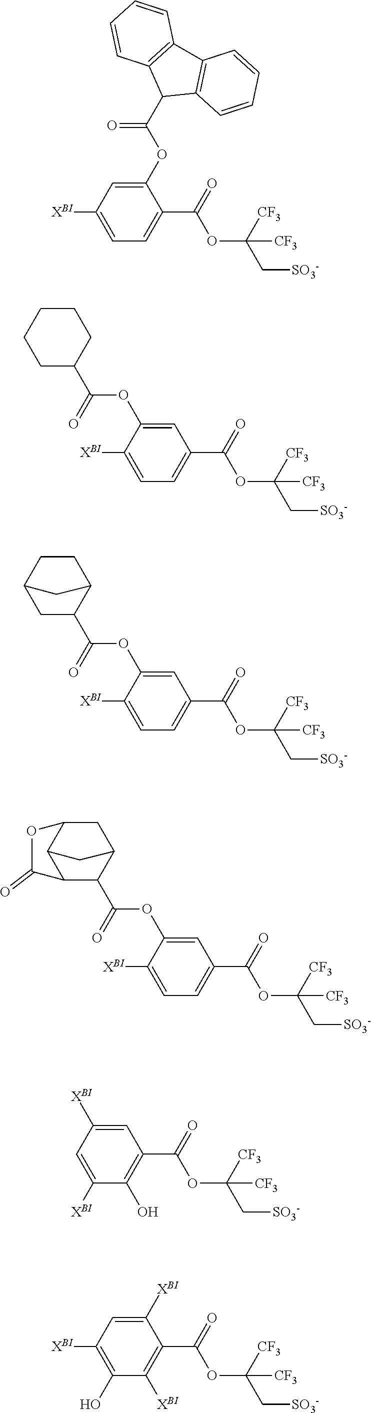

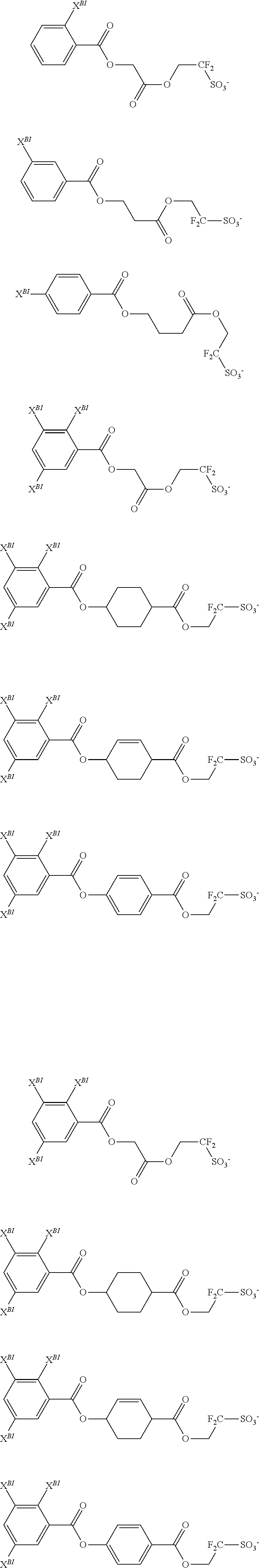

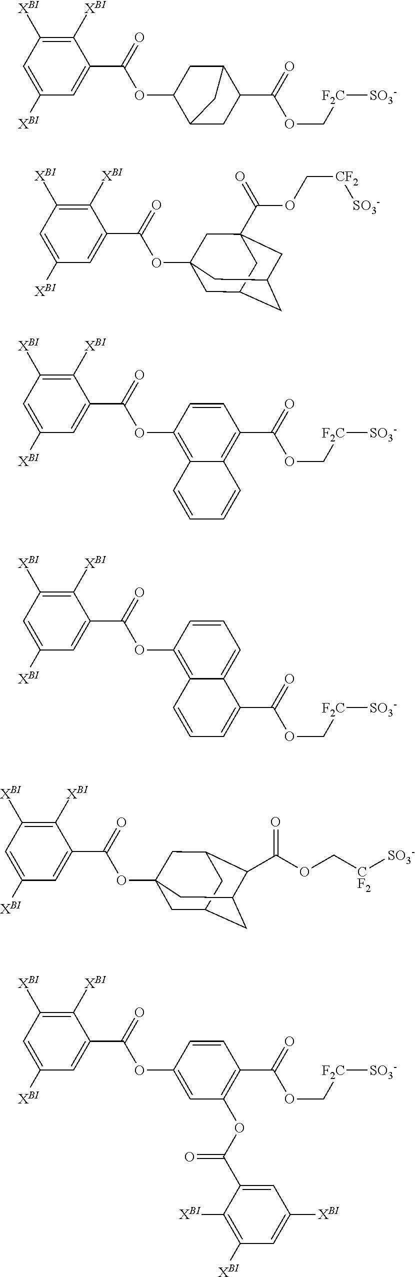

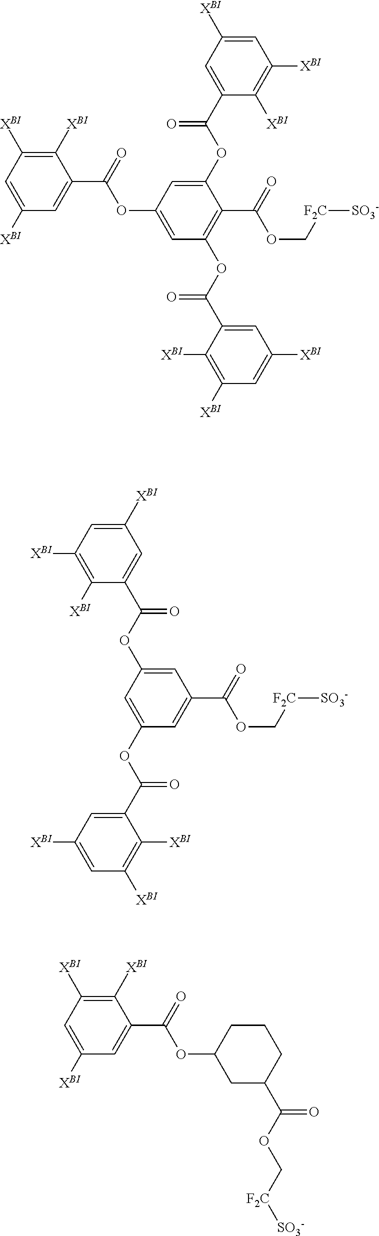

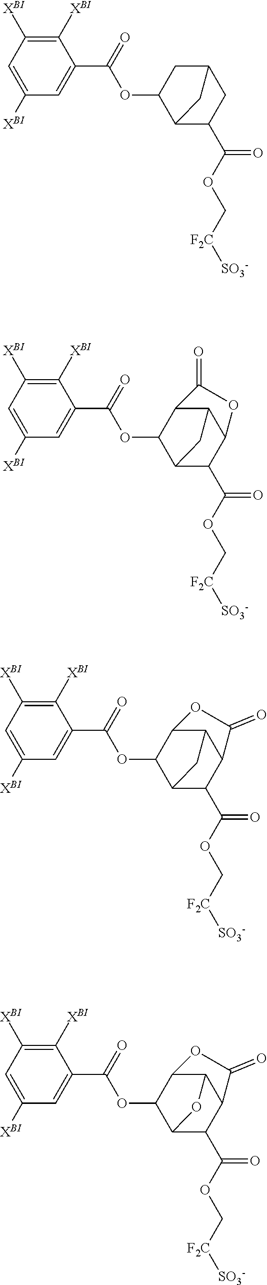

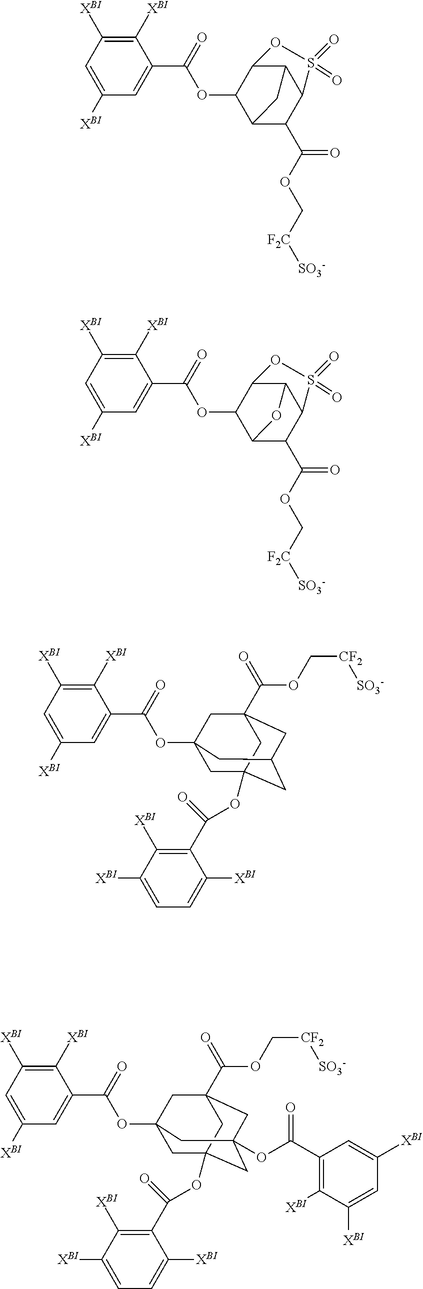

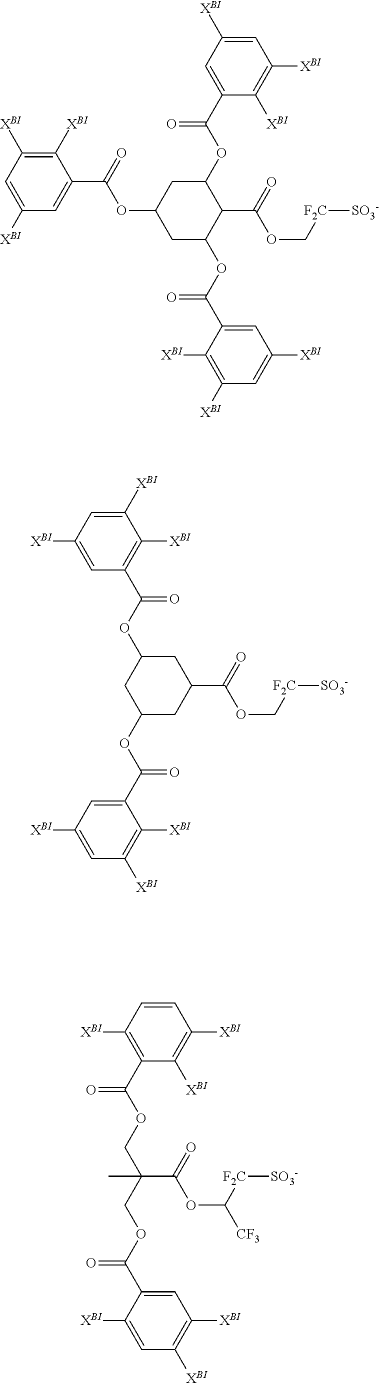

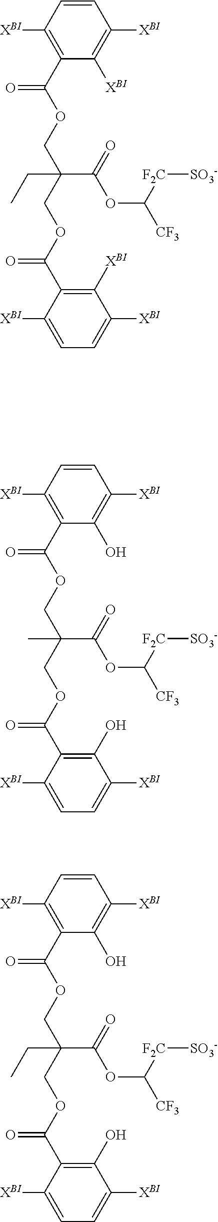

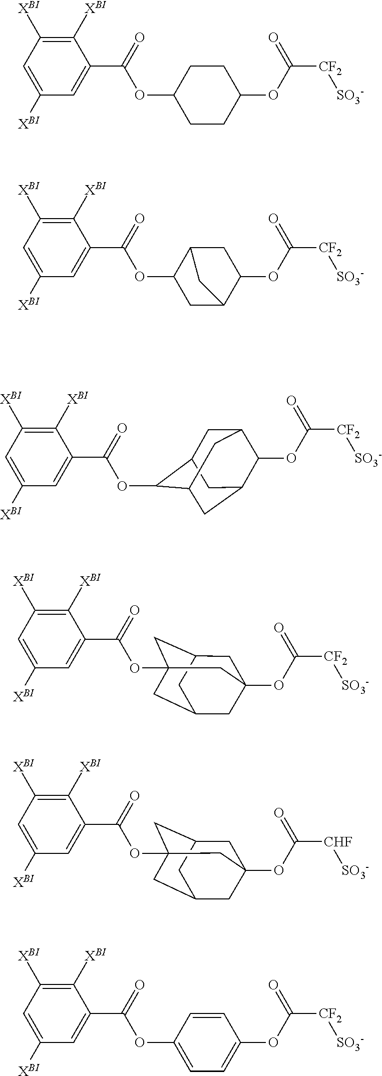

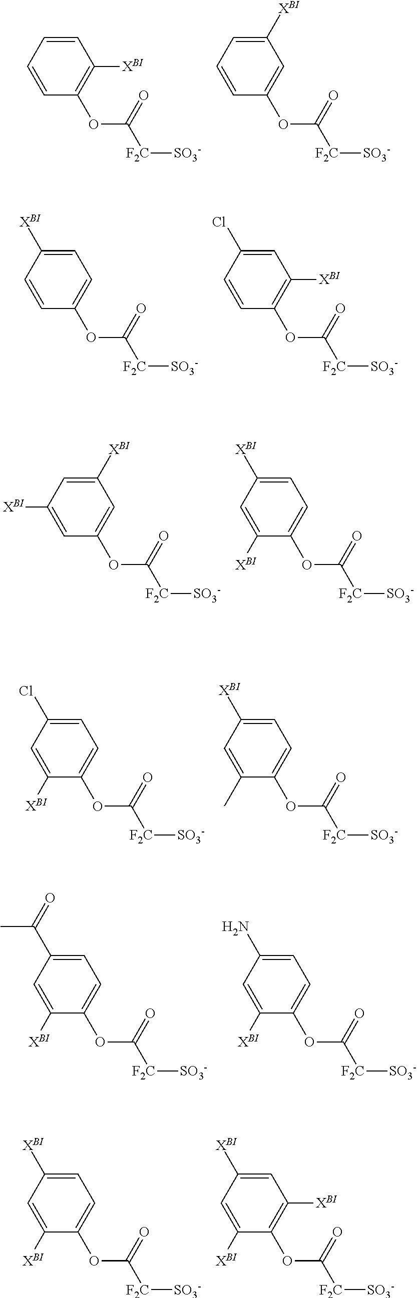

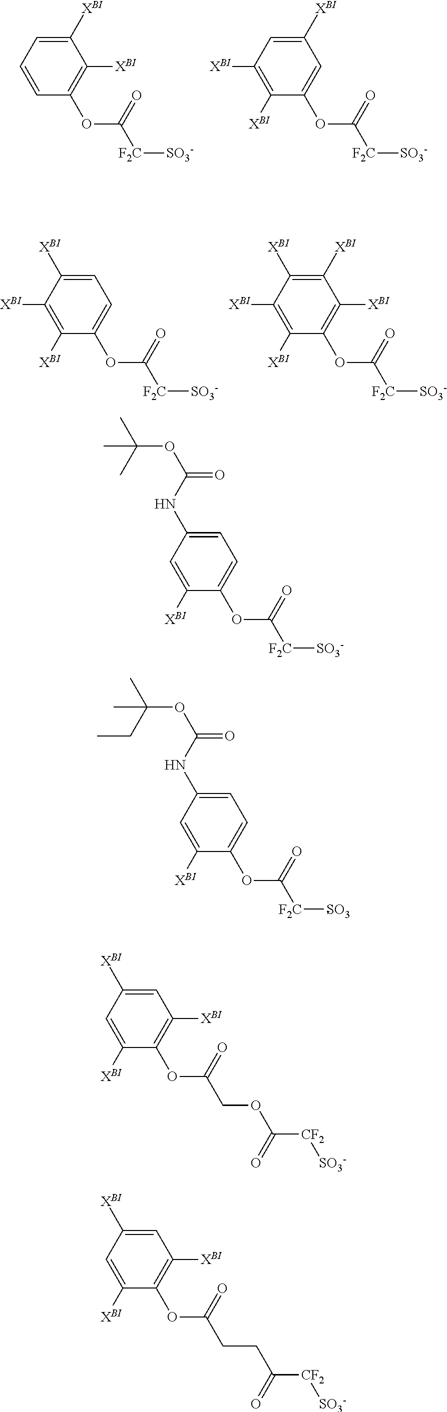

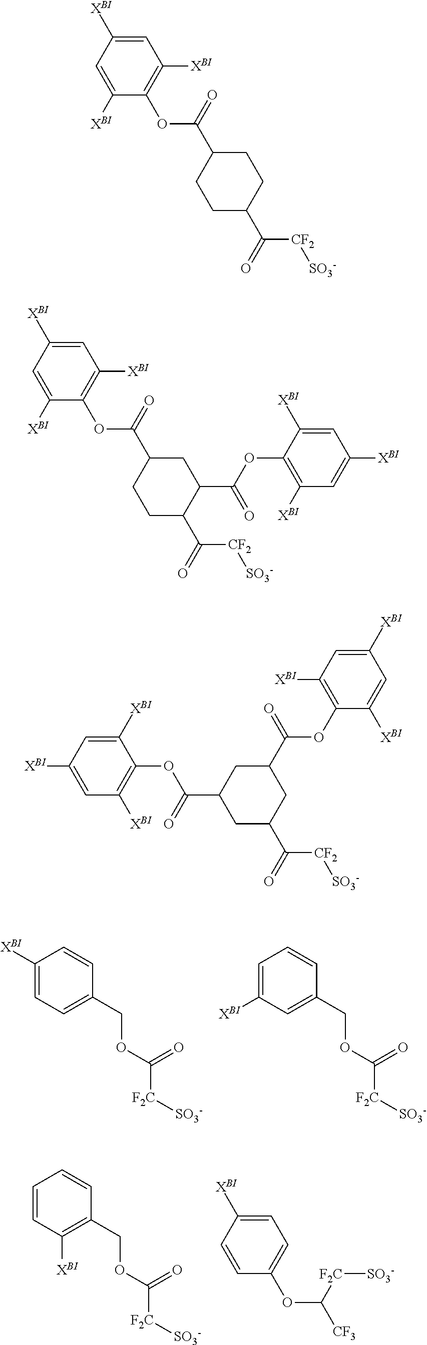

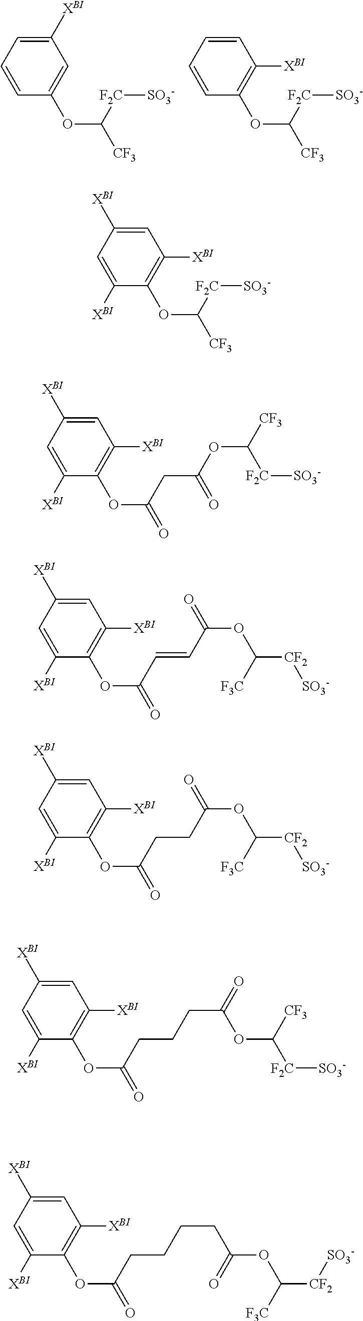

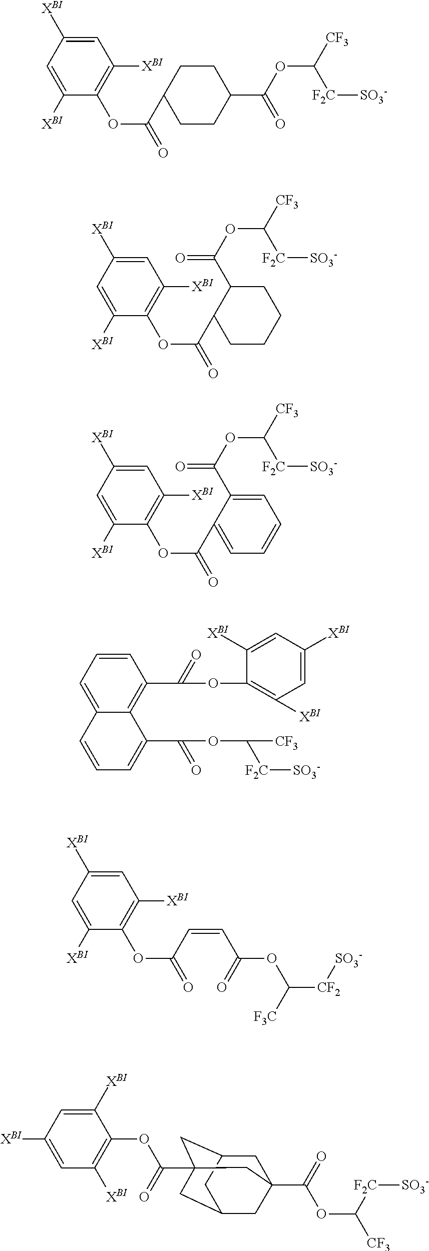

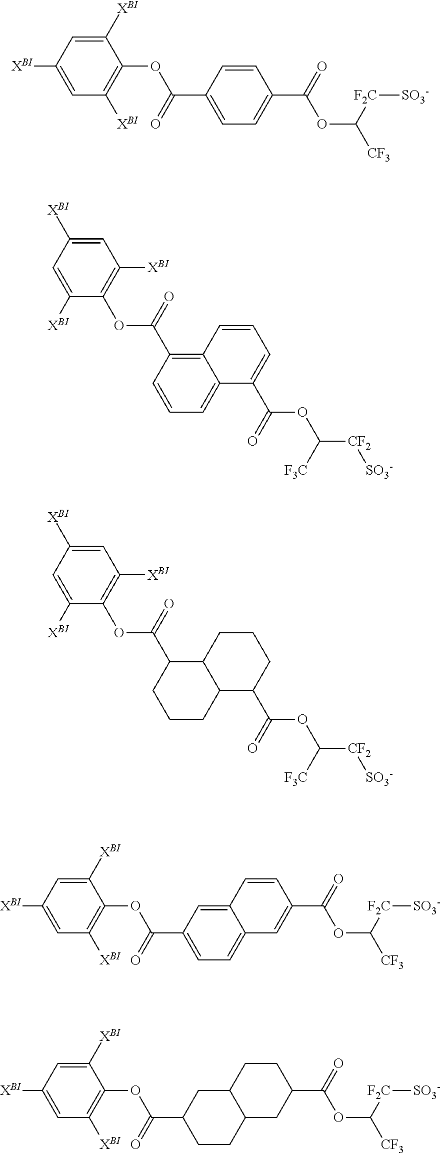

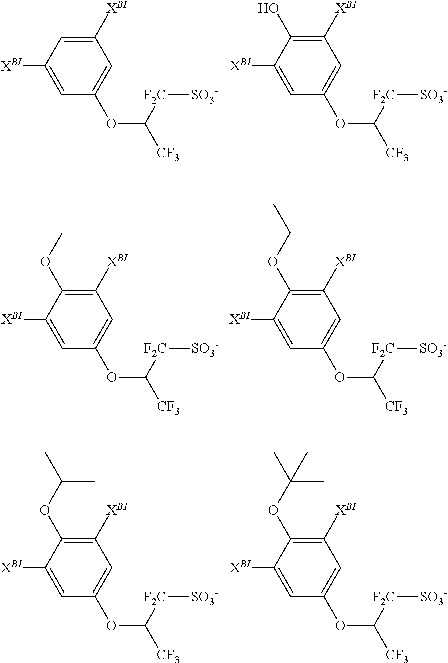

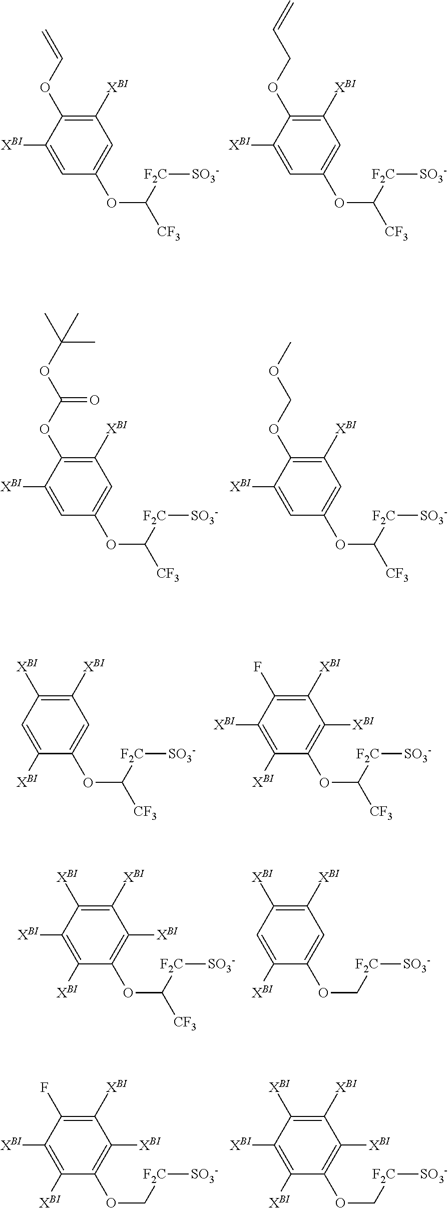

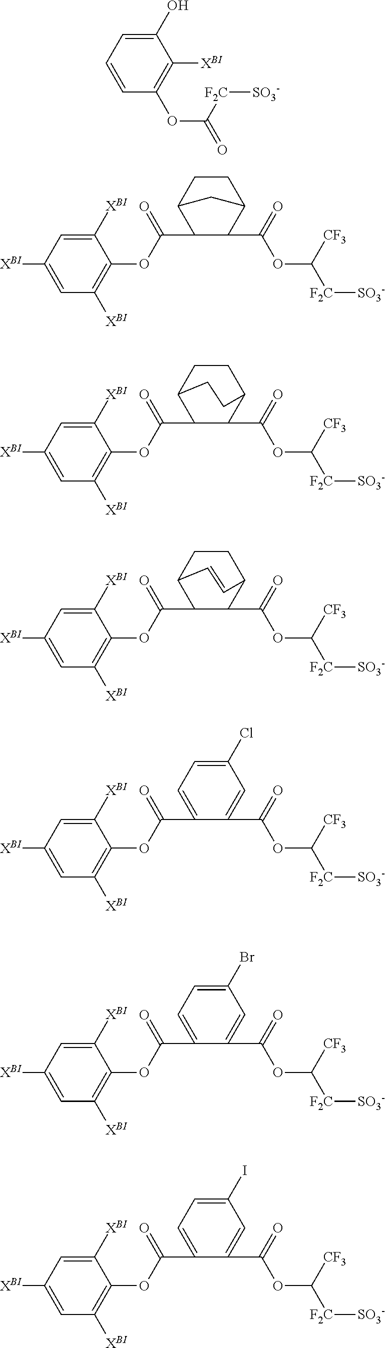

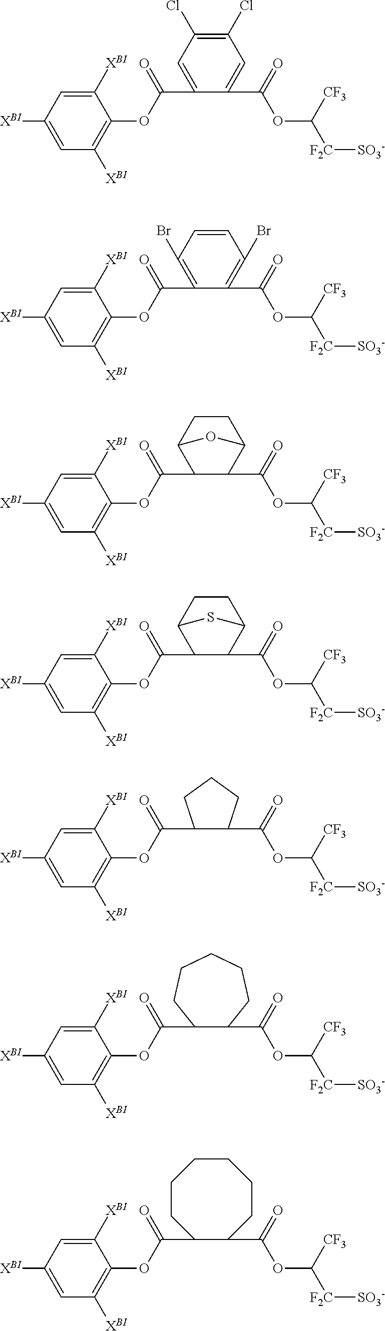

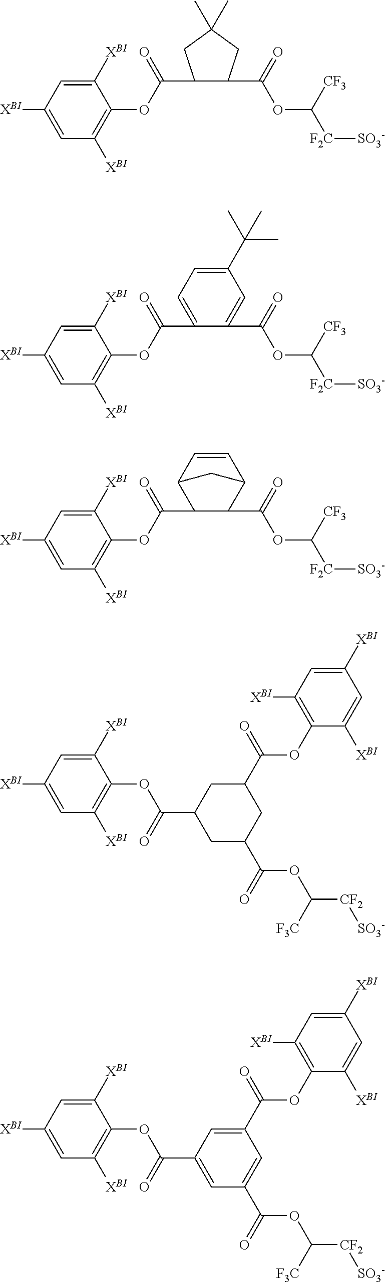

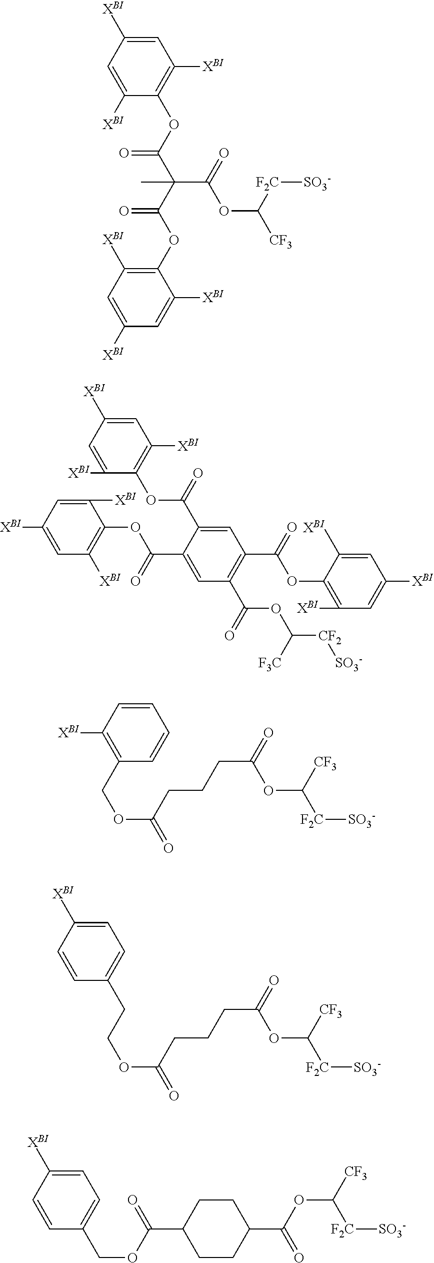

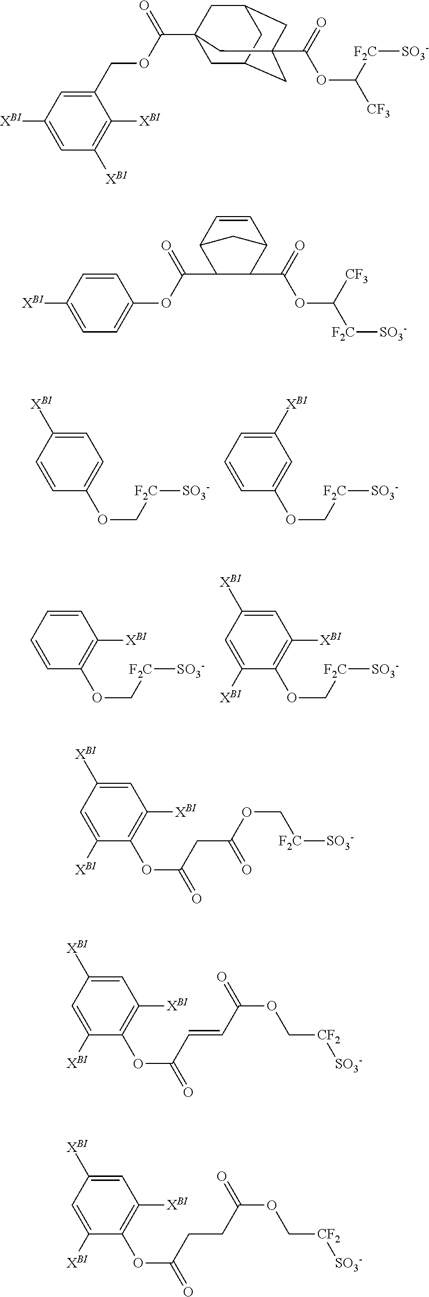

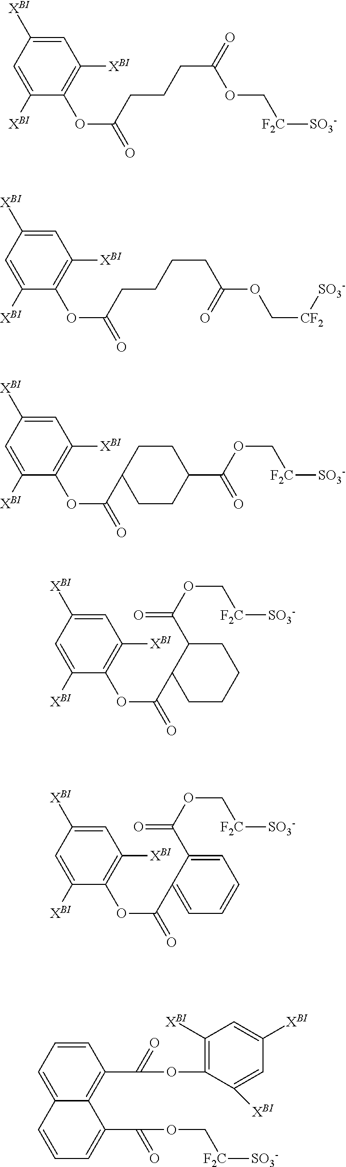

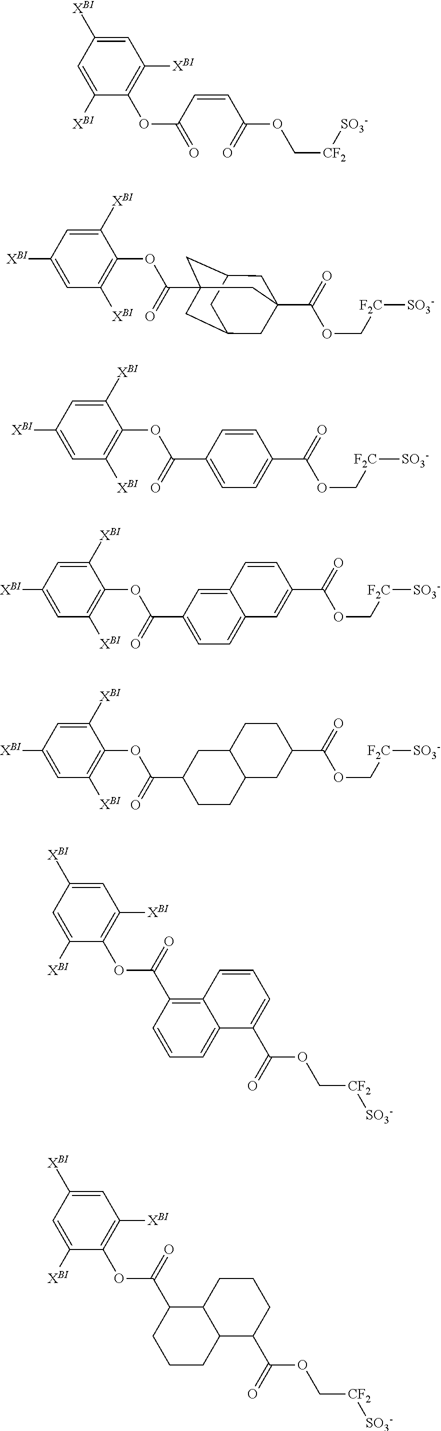

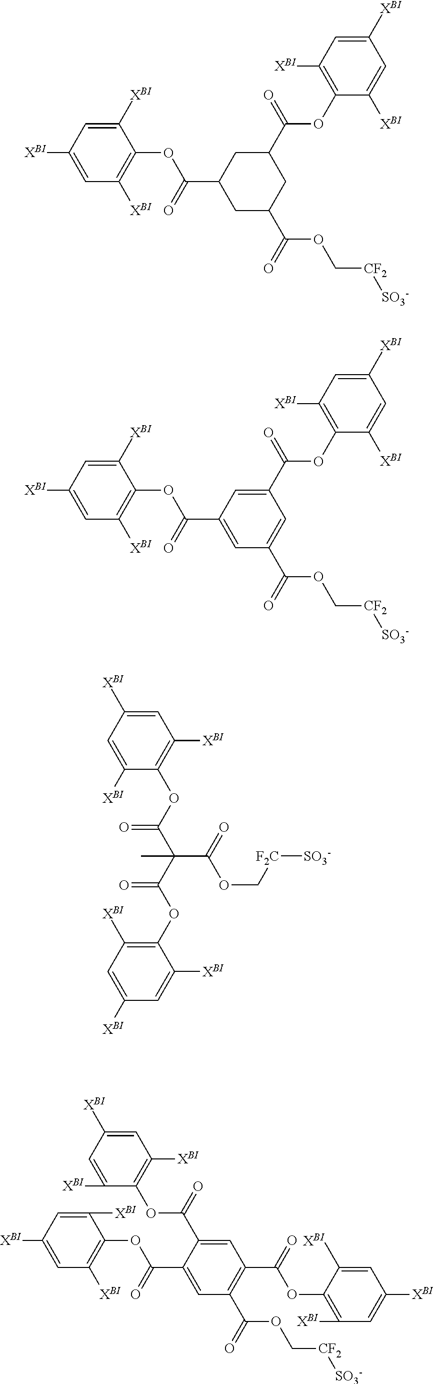

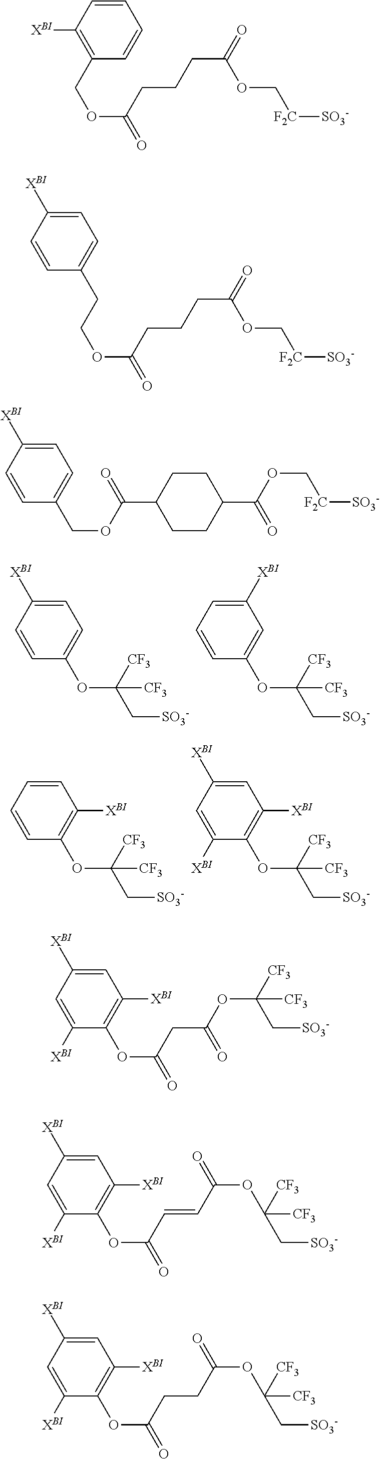

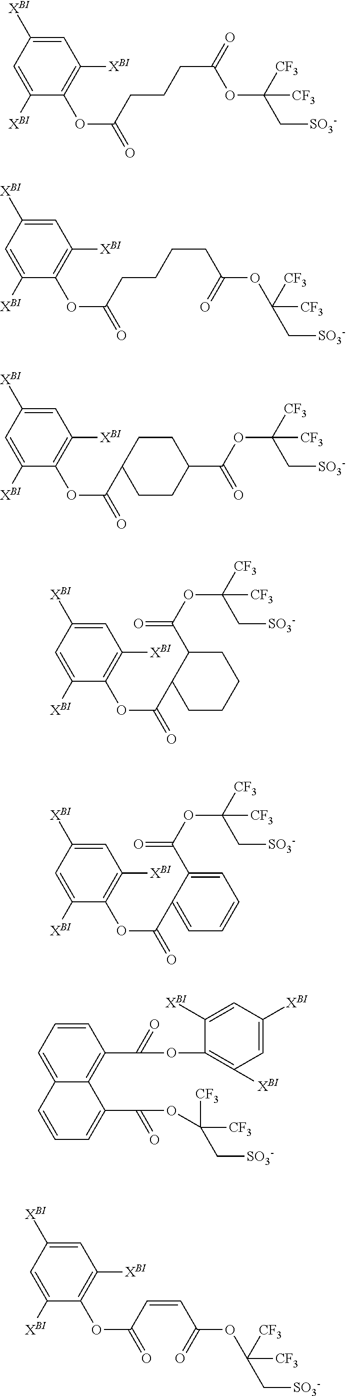

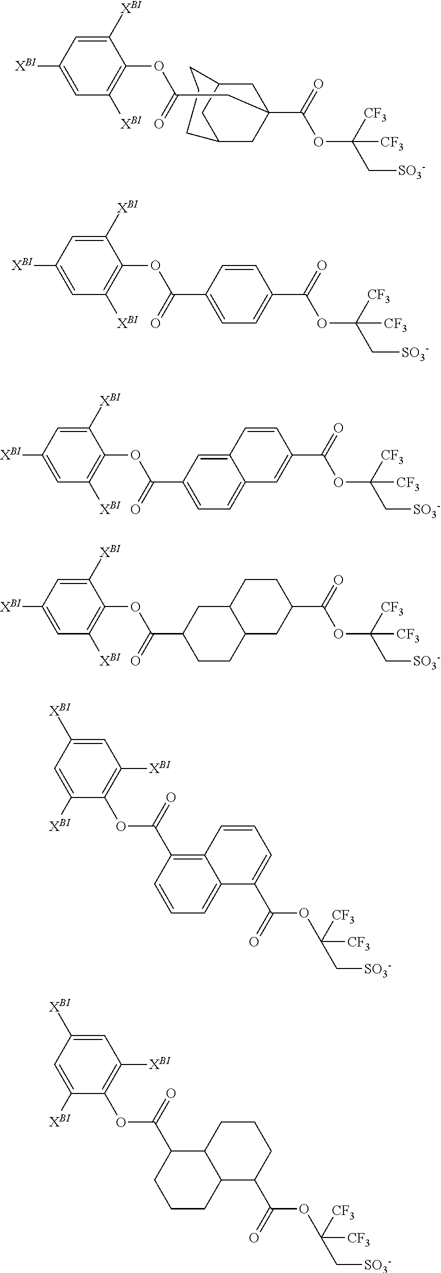

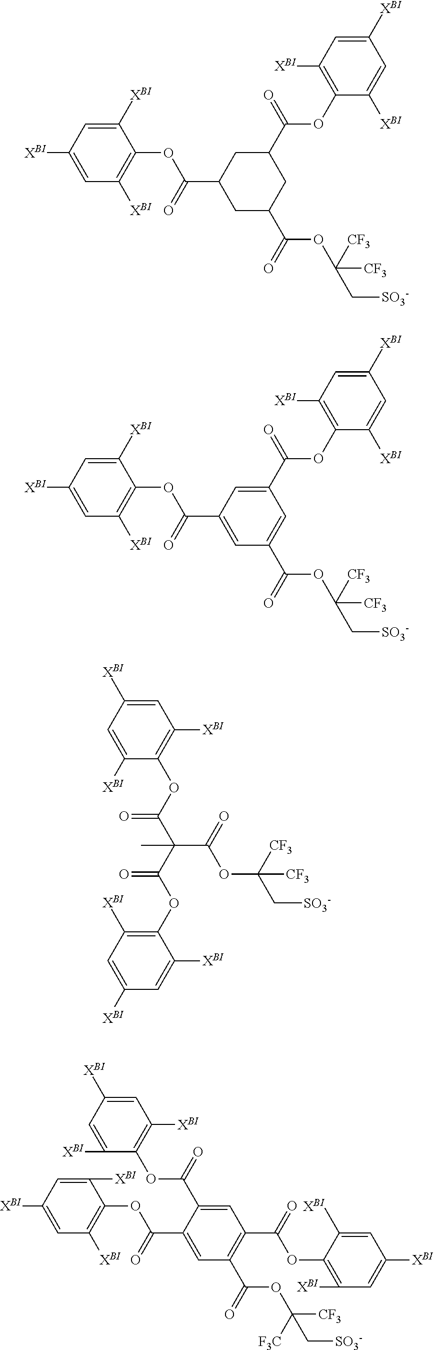

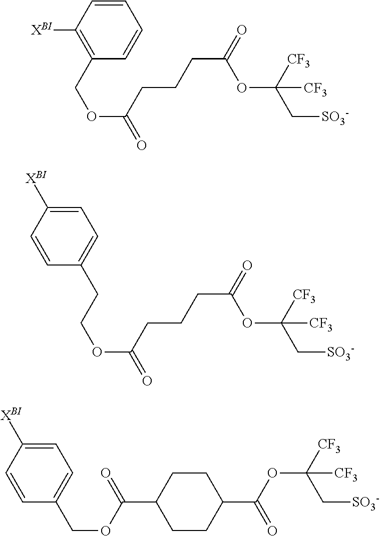

As the non-nucleophilic counter ion, fluorobenzenesulfonate anions having an iodized aromatic group bonded thereto as described in JP 6648726, anions having an acid-catalyzed decomposition mechanism as described in WO 2021/200056 and JP-A 2021-070692, anions having a cyclic ether group as described in JP-A 2018-180525 and JP-A 2021-035935, and anions as described in JP-A 2018-092159 can also be used.

Further, as the non-nucleophilic counter ion, bulky fluorine-free benzenesulfonate anions as described in JP-A 2006-276759, JP-A 2015-117200, JP-A 2016-065016, and JP-A 2019-202974, and fluorine-free benzenesulfonic acid or alkylsulfonate anions having an iodized aromatic group bonded thereto as described in JP 6645464 can also be used.

Furthermore, as the non-nucleophilic counter ion, bissulfonic acid anions as described in JP-A 2015-206932, sulfonamide or sulfonimide anions having sulfonic acid side and different side as described in WO 2020/158366, and anions having a sulfonic acid side and a carboxylic acid side as described in JP-A 2015-024989 can also be used.

Compounds having the formula (5) are also preferred as the photoacid generator (D).

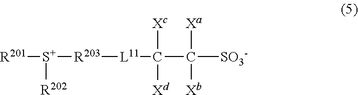

In the formula (5), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰² and R²⁰³ may bond together to form a ring with the sulfur atoms to which they are attached.

The C₁-C₃₀ hydrocarbyl groups represented by R²⁰¹ and R²⁰² may be saturated or unsaturated and straight, branched or cyclic. Examples thereof include C₁-C₃₀ alkyl groups such as methyl, ethyl, n-propyl, isopropyl, n-butyl, isobutyl, sec-butyl, tert-butyl, tert-pentyl, n-pentyl, n-hexyl, n-octyl, 2-ethylhexyl, n-nonyl, and n-decyl; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decyl, and adamantyl; C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl; and combinations thereof. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety.

The C₁-C₃₀ hydrocarbylene group represented by R²⁰³ may be saturated or unsaturated and straight, branched or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl; C₃-C₃₀ cyclic saturated hydrocarbylene groups such as cyclopentanediyl, cyclohexanediyl, norbornanediyl, and adamantanediyl; and arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene, and tert-butylnaphthylene. In the hydrocarbylene group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety. Of the heteroatoms, oxygen is preferred.

In the formula (5), L¹¹ is a single bond, ether bond or a C₁-C₂₀ hydrocarbylene group which may contain a heteroatom. The hydrocarbylene group may be saturated or unsaturated and straight, branched or cyclic. Examples thereof include those exemplified above for the hydrocarbylene group represented by R²⁰³.

In the formula (5), X^{a}, X^{b}, X^{c} and X^{d} are each independently hydrogen, fluorine or trifluoromethyl, at least one of X^{a}, X^{b}, X^{c} and X^{d} being fluorine or trifluoromethyl.

As the photoacid generator having the formula (5), a photoacid generator having the formula (5′) is preferred.

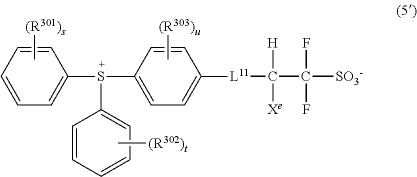

In the formula (5′), L¹¹ is as defined above. Xe is hydrogen or trifluoromethyl, preferably trifluoromethyl. R³⁰¹, R³⁰² and R³⁰³ are each independently hydrogen or a C₁-C₂₀ hydrocarbyl group which may contain a heteroatom. The hydrocarbyl group may be saturated or unsaturated and straight, branched or cyclic. Examples thereof include those exemplified above for the hydrocarbyl group represented by R^{fa1} in the formula (Xa-1-1). s and t are each independently 0, 1, 2, 3, 4, or 5, and u is 0, 1, 2, 3, or 4.

Examples of the photoacid generator having the formula (5) include those exemplified for the photoacid generator having the formula (2) in JP-A 2017-026980. Of the photoacid generators, those having an anion having the formula (Xa-1-1) or (Xa-4) are especially preferred because of reduced acid diffusion and high solubility in solvents. Also those having the formula (5′) are especially preferred because of extremely reduced acid diffusion.

When the chemically amplified resist composition of the invention comprises the photoacid generator (D), the amount thereof is preferably 0.1 to 40 parts by weight, more preferably 0.5 to 20 parts by weight per 80 parts by weight of the base polymer (A). As long as the amount of the photoacid generator (D) is in the range, good resolution is achievable and the risk of foreign matter being formed after development or during stripping of resist film is avoided. The photoacid generator (D) may be used alone or in combination of two or more.

### (E) Surfactant

The chemically amplified resist composition of the invention may further comprise a surfactant as component (E). The surfactant (E) is preferably a surfactant which is insoluble or substantially insoluble in water and soluble in alkaline developer, or a surfactant which is insoluble or substantially insoluble in water and alkaline developer. For the surfactant, reference may be made to those described in JP-A 2010-215608 and JP-A 2011-016746.

Of the surfactants described in the patent documents cited herein, preferred surfactants which are insoluble or substantially insoluble in water and alkaline developer are surfactants FC-4430 (3M), Surflon® S-381 (AGC Seimi Chemical Co., Ltd.), Olfine® E1004 (Nissin Chemical Co., Ltd.), and KH-20 and KH-30 (AGC Seimi Chemical Co., Ltd.). Oxetane ring-opened polymers having the formula (surf-1) are also preferred.

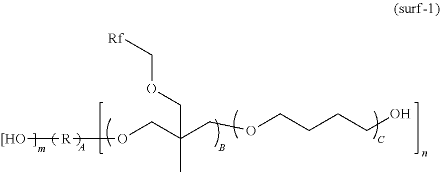

It is provided herein that R, Rf, A, B, C, m, and n are applied to only the formula (surf-1), independent of their descriptions other than for the surfactant. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

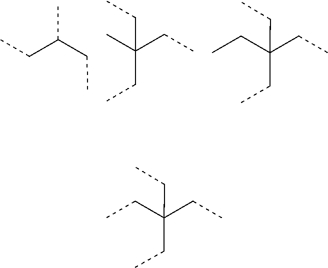

Herein the broken line is a valence bond. These formulae are partial structures derived from glycerol, trimethylol ethane, trimethylol propane, and pentaerythritol, respectively.

Among them, 1,4-butylene and 2,2-dimethyl-1,3-propylene are preferred.

Rf is trifluoromethyl or pentafluoroethyl, preferably trifluoromethyl. m is an integer of 0 to 3, n is an integer of 1 to 4, and the sum of m and n, which represents the valence of R, is an integer of 2 to 4. A is 1. B is an integer of 2 to 25, preferably an integer of 4 to 20. C is an integer of 0 to 10, preferably an integer of 0 or 1. Note that the formula (surf-1) does not prescribe the arrangement of respective constituent units while they may be arranged either blockwise or randomly. For the production of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the resist surface for achieving a function of minimizing water penetration or leaching. The surfactant is also effective for preventing water-soluble components from being leached out of the resist film for minimizing any damage to the exposure tool. The surfactant becomes solubilized during aqueous alkaline development following exposure or post exposure bake (PEB), and thus forms few or no foreign matter which become defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Examples of the polymeric surfactant include those containing repeat units of at least one type selected from the formulae (6A) to (6E).

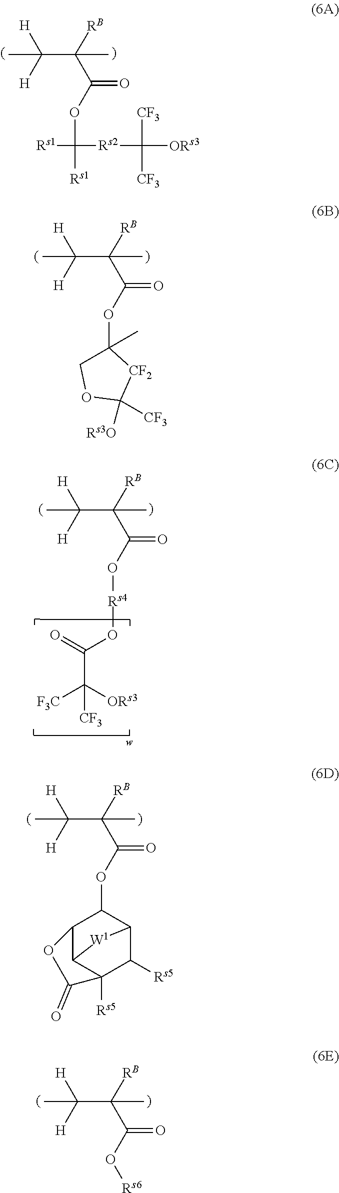

In the formulae (6A) to (6E), R^{B} is hydrogen, fluorine, methyl or trifluoromethyl. W¹ is —CH₂—, —CH₂CH₂—, —O—, or two separate —H. R^{s1} is each independently hydrogen or a C₁-C₁₀ hydrocarbyl group. R^{s2} is a single bond or a C₁-C₅ straight or branched hydrocarbylene group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, an ether bond or carbonyl moiety may intervene in a carbon-carbon bond. R^{s4} is a C₁-C₂₀ (w+1)-valent hydrocarbon or fluorinated hydrocarbon group, and w is 1, 2, or 3. R^{s5} is each independently hydrogen or a group represented by —C(═O)—O—R^{sa} wherein R^{sa} is a C₁-C₂₀ fluorinated hydrocarbyl group. R^{s6} is a C₁-C₁₅ hydrocarbyl or fluorinated hydrocarbyl group in which an ether bond or carbonyl moiety may intervene in a carbon-carbon bond.

The C₁-C₁₀ hydrocarbyl group represented by R^{s1} is preferably saturated and may be straight, branched or cyclic. Examples thereof include C₁-C₁₀ alkyl groups such as methyl, ethyl, n-propyl, isopropyl, n-butyl, isobutyl, sec-butyl, tert-butyl, n-pentyl, n-hexyl, n-heptyl, n-octyl, n-nonyl, and n-decyl, and C₃-C₁₀ cyclic saturated hydrocarbyl groups such as cyclopropyl, cyclobutyl, cyclopentyl, cyclohexyl, adamantyl, and norbornyl. Of these, C₁-C₆ hydrocarbyl groups are preferred.

The hydrocarbylene group represented by R^{s2} is preferably saturated and may be straight, branched or cyclic. Examples thereof include methylene, ethylene, propylene, butylene, and pentylene.

The hydrocarbyl group represented by R^{s3} or R^{s6} may be saturated or unsaturated and straight, branched or cyclic. Examples thereof include saturated hydrocarbyl groups, and aliphatic unsaturated hydrocarbyl groups such as alkenyl and alkynyl groups, with the saturated hydrocarbyl groups being preferred. Examples of the saturated hydrocarbyl groups include those exemplified for the hydrocarbyl group represented by R^{s1} as well as undecyl, dodecyl, tridecyl, tetradecyl, and pentadecyl. Examples of the fluorinated hydrocarbyl group represented by R^{s3} or R^{s6} include the foregoing hydrocarbyl groups in which some or all carbon-bonded hydrogen atoms are substituted by fluorine atoms. In these groups, an ether bond or carbonyl moiety may intervene in a carbon-carbon bond as mentioned above.

Examples of the acid labile group represented by R^{s3} include groups of the above formulae (AL-3) to (AL-5), trialkylsilyl groups in which each alkyl moiety has 1 to 6 carbon atoms, and C₄-C₂₀ oxoalkyl groups.

The (w+1)-valent hydrocarbon or fluorinated hydrocarbon group represented by R^{s4} may be straight, branched or cyclic, and examples thereof include the foregoing hydrocarbyl or fluorinated hydrocarbyl groups from which “w” number of hydrogen atoms are eliminated.

The fluorinated hydrocarbyl group represented by R^{s4} is preferably saturated and may be straight, branched or cyclic. Examples thereof include the foregoing hydrocarbyl groups in which some or all of the hydrogen atoms are substituted by fluorine atoms. Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the repeat units having any of the formulae (6A) to (6E) include those shown below, but not limited thereto. In the formulae, R^{B} is as defined above.

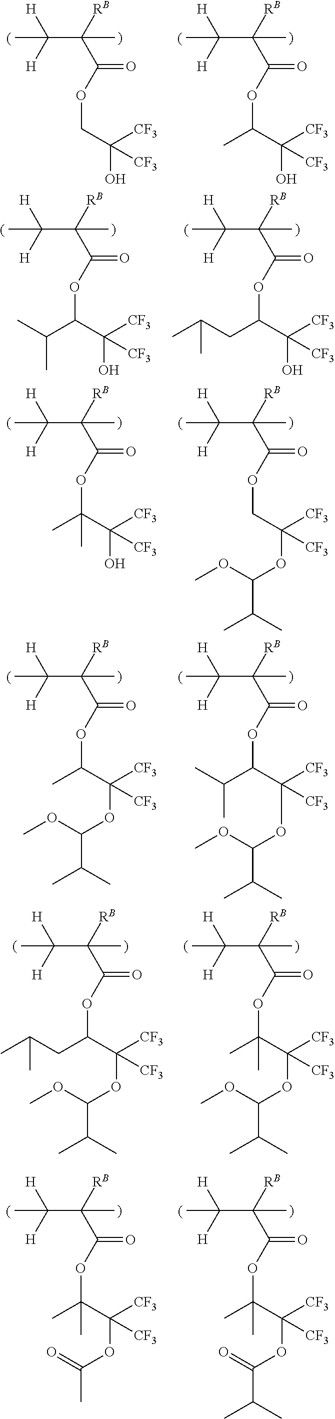

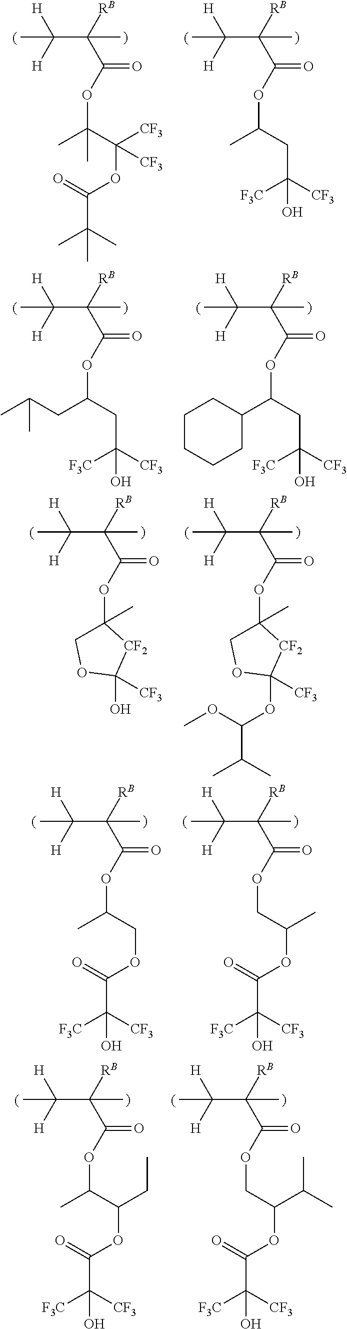

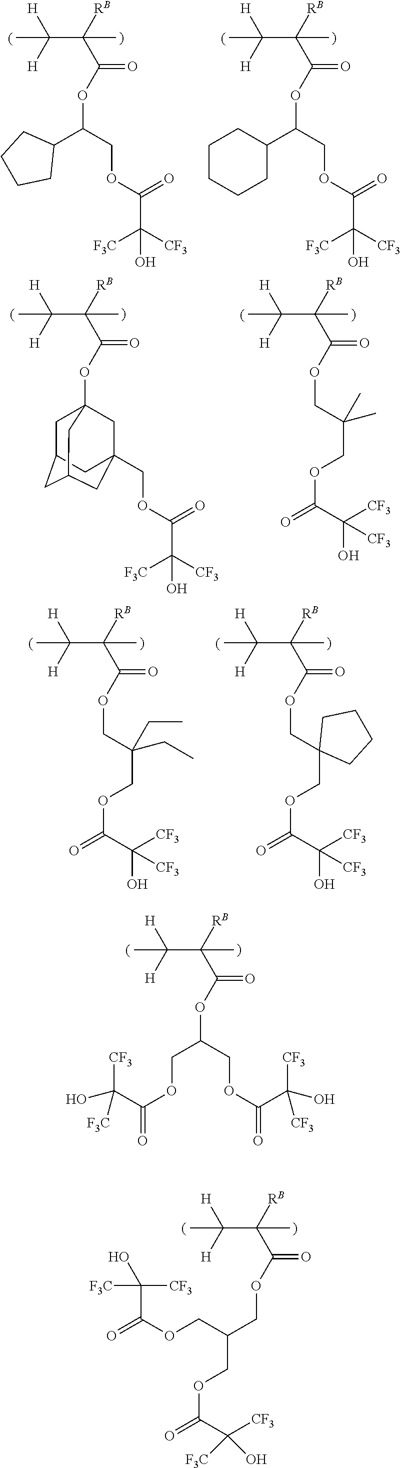

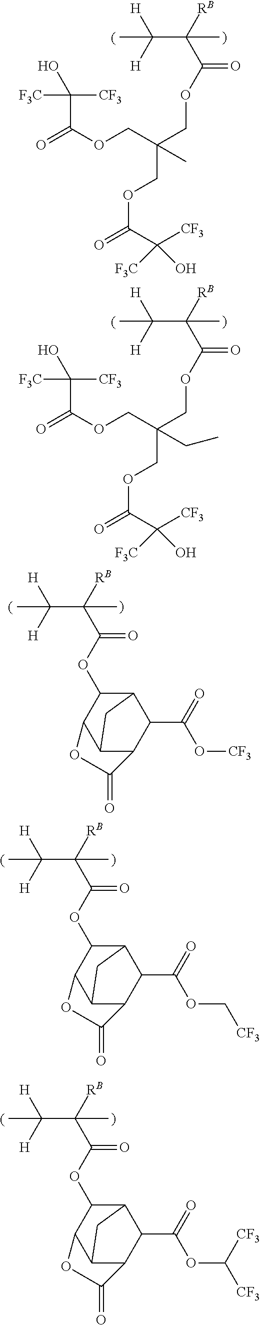

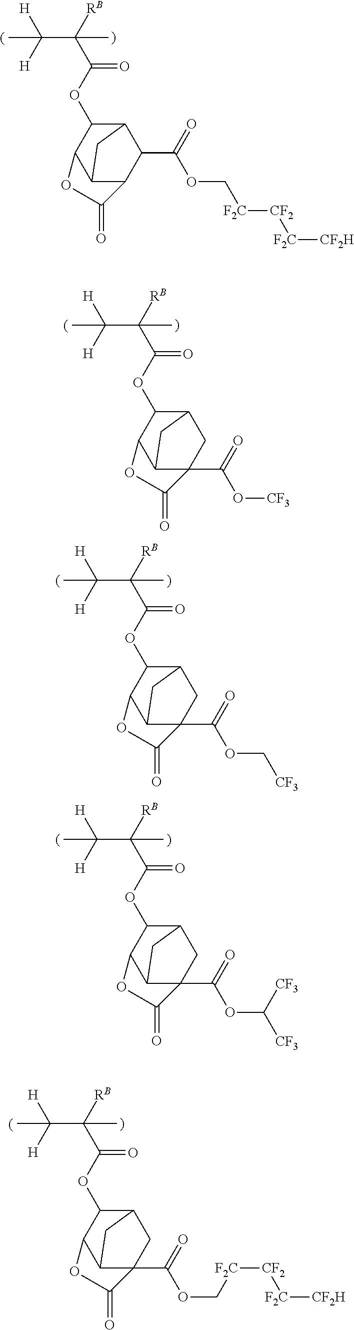

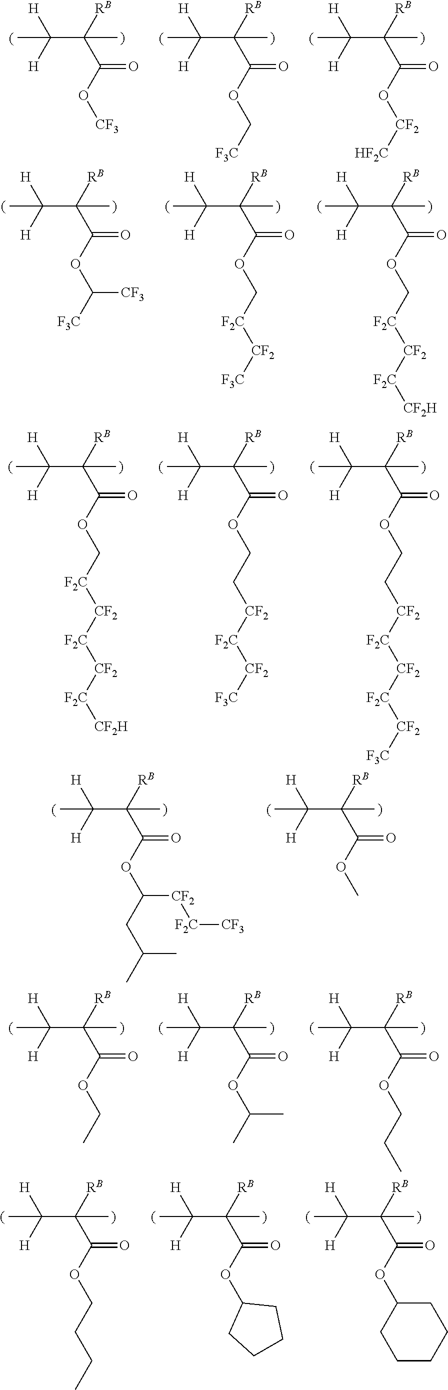

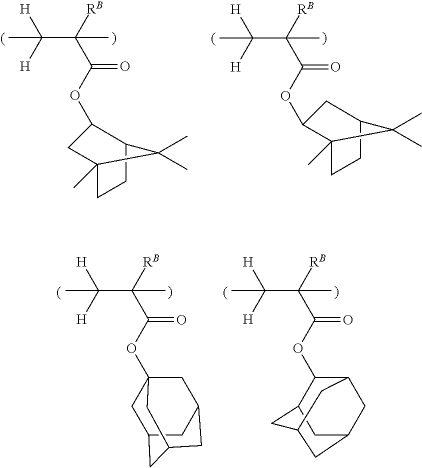

The polymeric surfactant may further contain repeat units other than the repeat units having the formulae (6A) to (6E). Examples of the other repeat units include those derived from methacrylic acid and α-trifluoromethylacrylic acid derivatives. In the polymeric surfactant, the content of the repeat units having the formulae (6A) to (6E) is preferably at least 20 mol %, more preferably at least 60 mol %, most preferably 100 mol % of the overall repeat units.

The polymeric surfactant has a Mw of preferably 1,000 to 500,000, more preferably 3,000 to 100,000, and a Mw/Mn of preferably 1.0 to 2.0, more preferably 1.0 to 1.6.

The polymeric surfactant may be synthesized, for example, by dissolving an unsaturated bond-containing monomer or monomers providing repeat units having the formulae (6A) to (6E) and optionally other repeat units in an organic solvent, adding a radical initiator, and heating for polymerization. Examples of the organic solvents used in the polymerization include toluene, benzene, THF, diethyl ether, and dioxane. Examples of the polymerization initiator include AIBN, 2,2′-azobis(2,4-dimethylvaleronitrile), dimethyl 2,2-azobis(2-methylpropionate), benzoyl peroxide, and lauroyl peroxide. Preferably, the reaction temperature is 50 to 100° C. and the reaction time is 4 to 24 hours. The acid labile group that has been incorporated in the monomer may be kept as such, or polymerization may be followed by protection or partial protection.

During the synthesis of polymeric surfactant, any known chain transfer agent such as dodecyl mercaptan or 2-mercaptoethanol may be added for molecular weight control purpose. The amount of chain transfer agent added is preferably 0.01 to 10 mol % based on the total moles of monomers to be polymerized.

When the chemically amplified resist composition of the invention comprises the surfactant (E), the amount thereof is preferably 0.1 to 50 parts by weight, more preferably 0.5 to 10 parts by weight per 80 parts by weight of the base polymer (A). At least 0.1 parts by weight of the surfactant (E) is effective in improving the receding contact angle with water of the resist film at its surface. Up to 50 parts by weight of the surfactant (E) is effective in forming a resist film having a low rate of dissolution in a developer and capable of maintaining the height of a small-size pattern formed therein. The surfactant (E) may be used alone or in combination of two or more.

### (F) Other Components

The chemically amplified resist composition of the invention may further comprise (F) other components, for example, a compound which is decomposed with an acid to generate another acid (i.e., acid amplifier compound), an organic acid derivative, a fluorinated alcohol, and a compound having a Mw of up to 3,000 which changes its solubility in developer under the action of an acid (i.e., dissolution inhibitor). For the acid amplifier compound, reference may be made to compounds described in JP-A 2009-269953 and JP-A 2010-215608. When the chemically amplified resist composition of the invention comprises the acid amplifier compound, the amount thereof is preferably 0 to 5 parts by weight, more preferably 0 to 3 parts by weight per 80 parts by weight of the base polymer (A). An extra amount of the acid amplifier compound can make the acid diffusion control difficult and cause degradations to resolution and pattern profile. For the organic acid derivative, fluorinated alcohol, and dissolution inhibitor, reference should be made to compounds described in JP-A 2009-269953 and JP-A 2010-215608.

### Pattern Forming Process

A pattern forming process of the invention comprises the steps of applying the chemically amplified resist composition defined above onto a substrate to form a resist film thereon, exposing the resist film to high-energy radiation, and developing the exposed resist film in a developer.

The substrate used herein may be a substrate for integrated circuitry fabrication, e.g., Si, SiO₂, SiN, SION, TIN, WSi, BPSG, SOG, organic antireflective film, etc. or a substrate for mask circuitry fabrication, e.g., Cr, CrO, CrON, MoSi₂, SiO₂, etc.

The chemically amplified resist composition is applied onto a substrate by a suitable coating technique such as spin coating. The coating is prebaked on a hot plate preferably at a temperature of 60 to 150° C. for 1 to 10 minutes, more preferably at 80 to 140° C. for 1 to 5 minutes. The resulting resist film preferably has a thickness of 0.05 to 2 μm.

Examples of the high-energy radiation used for exposure of the resist film include KrF excimer laser, ArF excimer laser, EB, and EUV of wavelength 3 to 15 nm. On use of KrF excimer laser, ArF excimer laser or EUV, the resist film is exposed through a mask having a desired pattern, in a dose of preferably 1 to 200 mJ/cm², more preferably 10 to 100 mJ/cm². On use of EB, a pattern may be written directly or through a mask having the desired pattern, in a dose of preferably 1 to 300 μC/cm², more preferably 10 to 200 μC/cm².

The exposure may be performed by conventional lithography whereas the immersion lithography of holding a liquid having a refractive index of at least 1.0 between the resist film and the projection lens may be employed. In this case, a protective film which is insoluble in water may be also used.

While the water-insoluble protective film serves to prevent any components from being leached out of the resist film and to improve water sliding on the film surface, it is generally divided into two types. The first type is an organic solvent-strippable protective film which must be stripped, prior to aqueous alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

After the exposure, the resist film may be baked (PEB), for example, on a hotplate preferably at 60 to 150° C. for 1 to 5 minutes, more preferably at 80 to 140° C. for 1 to 3 minutes.

The resist film is then developed with a developer in the form of an aqueous alkaline solution, for example, preferably 0.1 to 5 wt %, more preferably 2 to 3 wt % aqueous solution of tetramethylammonium hydroxide (TMAH) for preferably 0.1 to 3 minutes, more preferably 0.5 to 2 minutes by conventional techniques such as dip, puddle and spray techniques. In this way, the exposed region of the resist film is dissolved away, and a desired pattern is formed on the substrate.

Any desired step may be added to the pattern forming process. For example, after the resist film is formed, a step of rinsing with pure water may be introduced to extract the acid generator or the like from the film surface or wash away particles. After exposure, a step of rinsing may be introduced to remove any water remaining on the film after exposure.

Also, a double patterning process may be used for pattern formation. The double patterning process includes a trench process of processing an underlay to a 1:3 trench pattern by a first step of exposure and etching, shifting the position, and forming a 1:3 trench pattern by a second step of exposure, for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the pattern forming process, negative tone development may also be used. That is, an organic solvent may be used instead of the aqueous alkaline solution as the developer for developing and dissolving away the unexposed region of the resist film.

The organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, and 2-phenylethyl acetate. These organic solvent may be used alone or in admixture of two or more.

### EXAMPLES

Synthesis Examples, Examples and Comparative Examples are given below by way of illustration and not by way of limitation. Analysis is made by time-of-flight mass spectrometry using the following instrument.

### MALDI TOF-MS: S3000 by JEOL Ltd.

### [1] Synthesis of Sulfonium Salt Monomer

### Example 1-1

### Synthesis of Monomer PAG-1

(1) Synthesis of Intermediate in-1

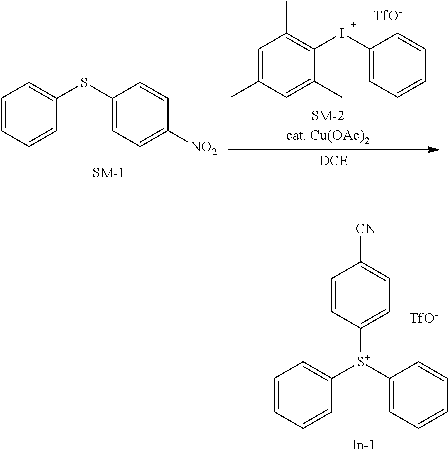

Under nitrogen atmosphere, 34.7 g of SM-1, 85.0 g of SM-2, and 8.24 g of copper acetate were dissolved in 300 g of dichloroethane. Thereafter, the reaction system was heated to 100° C. and aged for 15 hours. After aging, the reaction system was cooled, and 150 g of water was added to stop the reaction. Thereafter, the organic layer was separated, washed with water, and then concentrated under reduced pressure to distill off the solvent. The residue was recrystallized from diisopropyl ether, obtaining intermediate In-1 as yellow crystals (amount 42.5 g, yield: 62%).

### (2) Synthesis of Monomer PAG-1

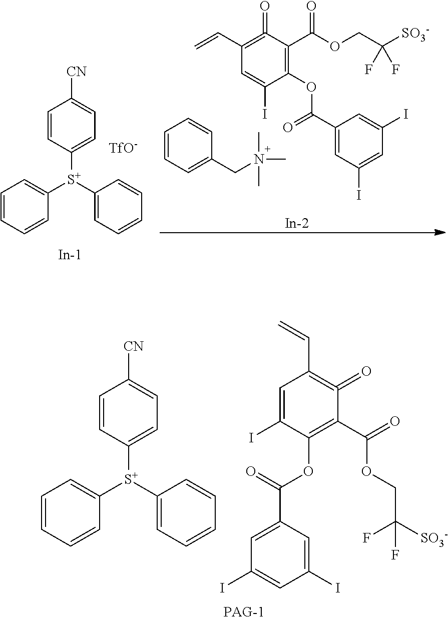

Under nitrogen atmosphere, 37.7 g of intermediate In-1, 70.4 g of intermediate In-2, 300 g of methylene chloride and 150 g of water were charged, and the mixture was stirred at room temperature for 30 minutes. The organic layer was separated, washed with water, and then concentrated under reduced pressure. The residue was washed with diisopropyl ether and concentrated, obtaining monomer PAG-1 as an oily product (amount 79.0 g, yield 96%).

The TOF-MS results for PAG-1 are shown below.

### MALDI TOF-MS:

positive M⁺ 308 (corresponding to C₁₈H₁₄NO₂S⁺)
negative M⁻ 789 (corresponding to C₁₈H₁₀F₂I₃O_{7S}⁻)

### Examples 1-2 to 1-9

### Synthesis of Monomers PAG-2 to PAG-9

The following sulfonium salt monomers PAG-2 to PAG-9 were synthesized by corresponding raw materials and various organic synthesis reactions.

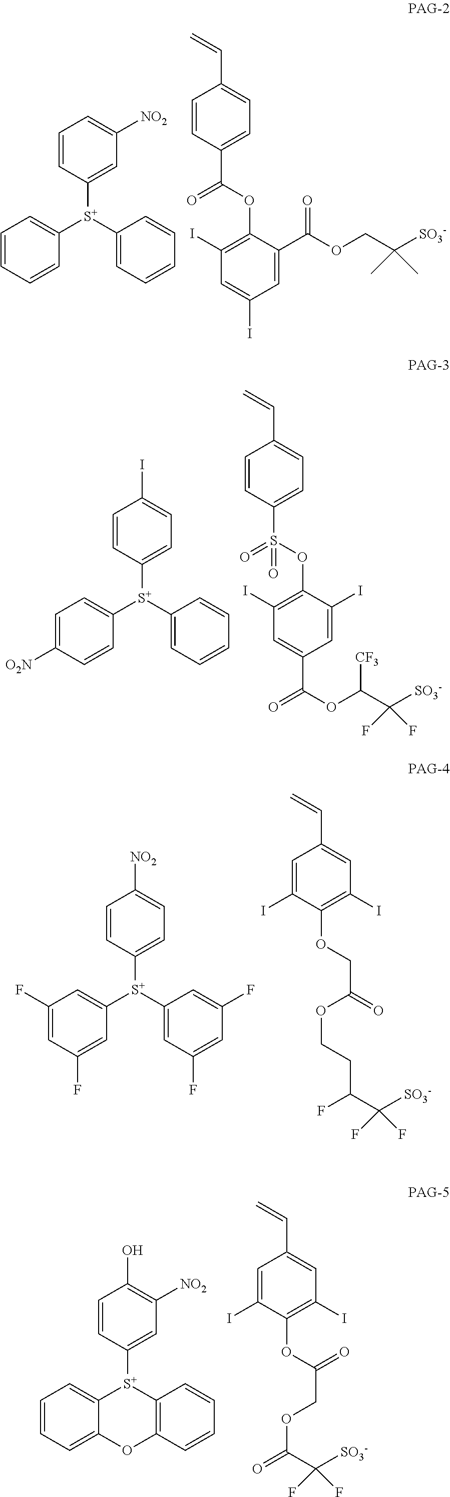

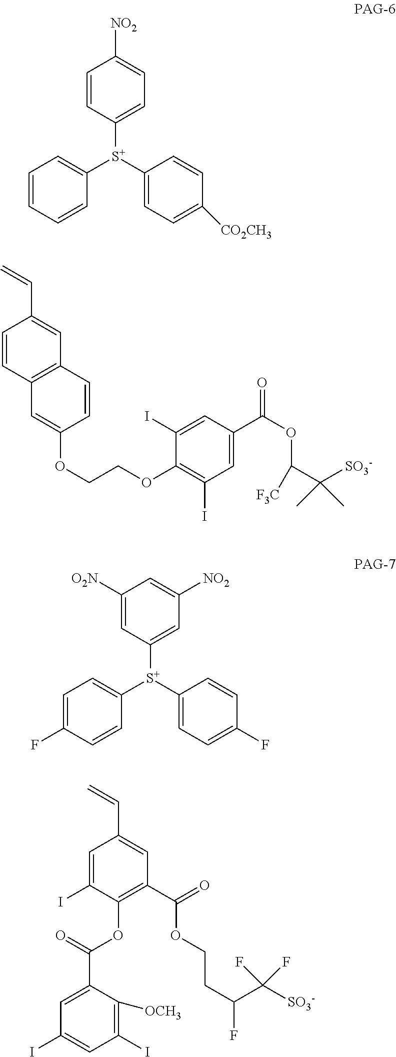

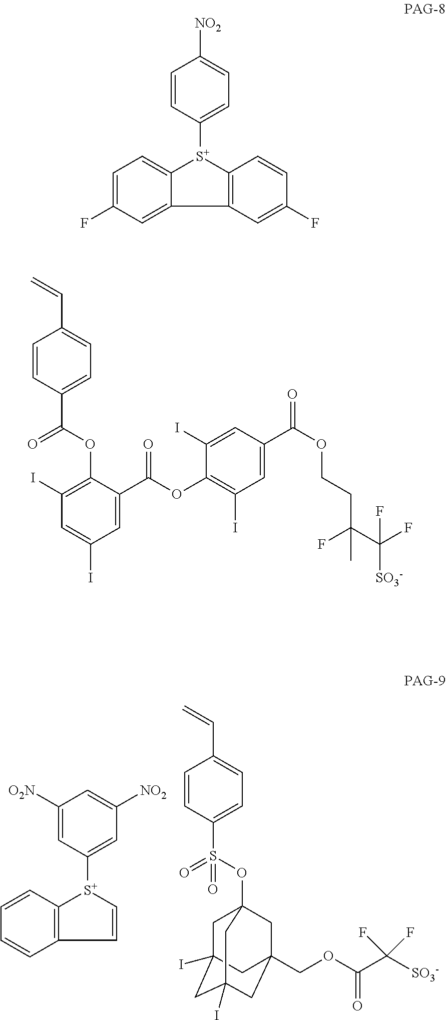

### Comparative Examples 1-1 to 1-6

### Synthesis of Comparative Monomers PAG-A to PAG-F

Comparative sulfonium salt monomers PAG-A to PAG-F shown below were synthesized by corresponding raw materials and various organic synthesis reactions.

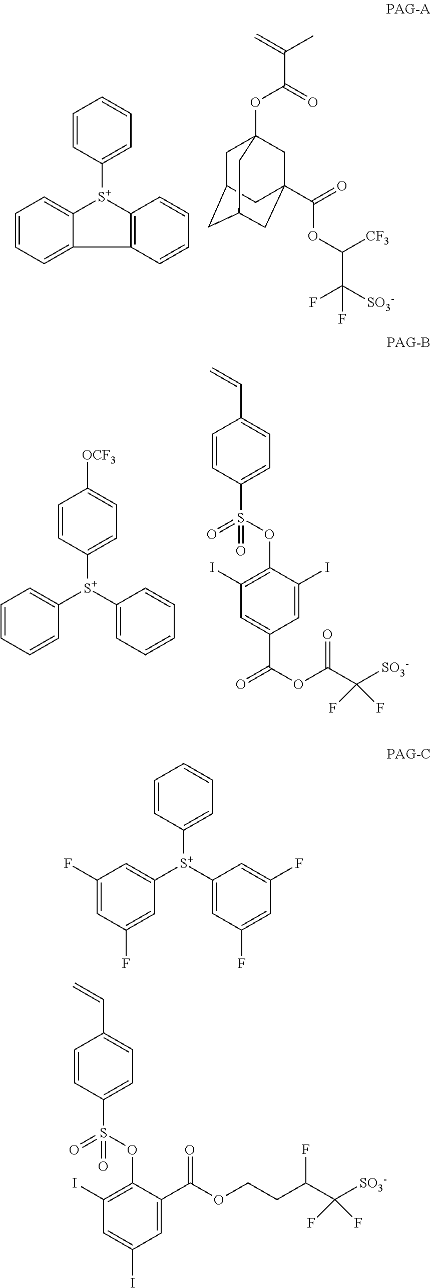

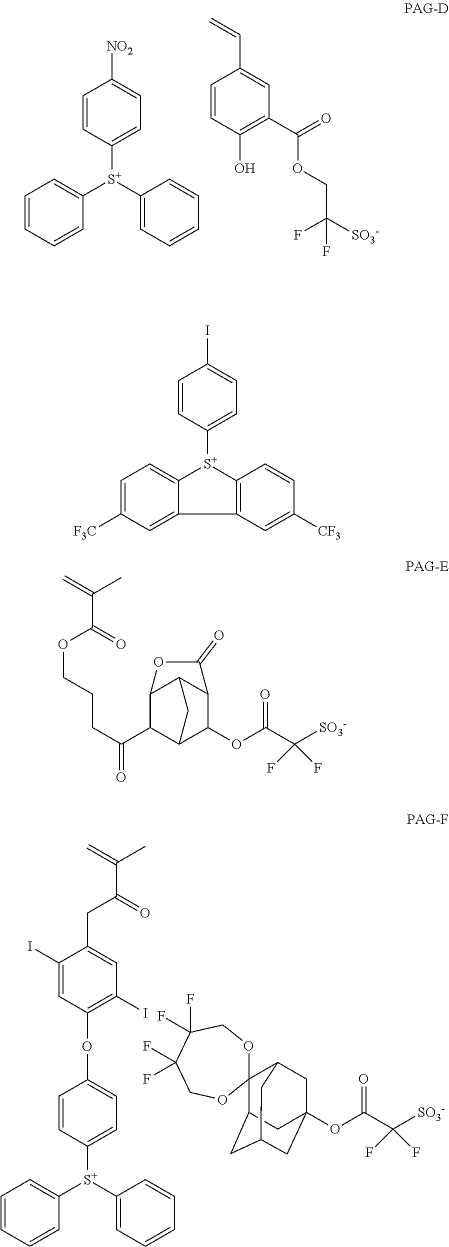

### [2] Synthesis of Base Polymers

Among the monomers used for synthesis of base polymers, monomers other than PAG-1 to PAG-9 and PAG-A to PAG-F are as follows.

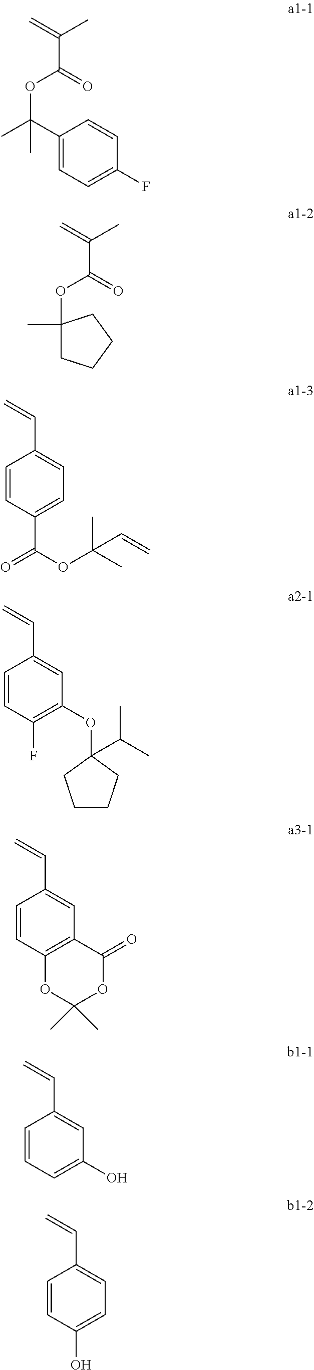

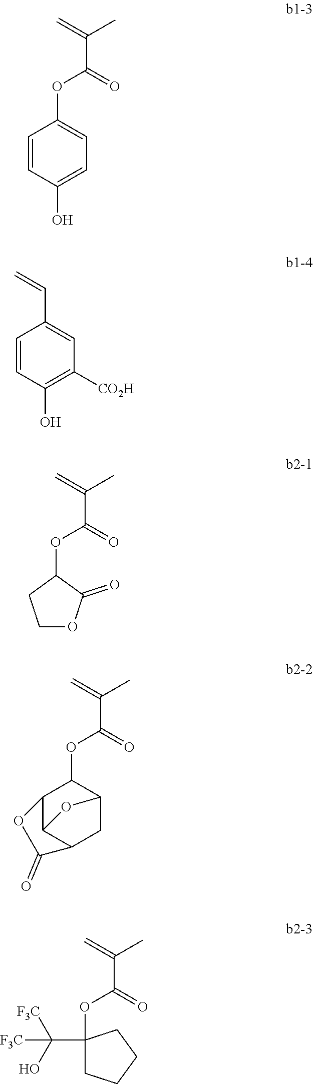

### Example 2-1

### Synthesis of Polymer P-1

In a flask under nitrogen atmosphere, 19.0 g of monomer a1-1, 5.6 g of monomer b1-1, 25.5 g of monomer PAG-1, 1.78 g of V-601 (manufactured by Wako Pure Chemical Industries, Ltd.) and 70 g of MEK were placed to prepare a monomer-polymerization initiator solution. In another flask under nitrogen atmosphere, 23 g of MEK was placed and heated to 80° C. with stirring, and then the monomer-polymerization initiator solution was added dropwise thereto over 4 hours. After completion of the dropwise addition, stirring was continued for 2 hours while maintaining the temperature of the polymerization solution at 80° C., and then the polymerization solution was cooled to room temperature. The obtained polymerization solution was added dropwise to 1,000 g of vigorously stirred hexane, and the precipitated polymer was separated by filtration. Further, the obtained polymer was washed with 300 g of hexane twice, and then vacuum-dried at 50° C. for 20 hours to obtain white powdery polymer P-1 (amount 49.1 g, yield 98%). The polymer P-1 has a Mw of 9,500 and a Mw/Mn of 1.62. Mw is measured by GPC versus polystyrene standards using a DMF solvent.

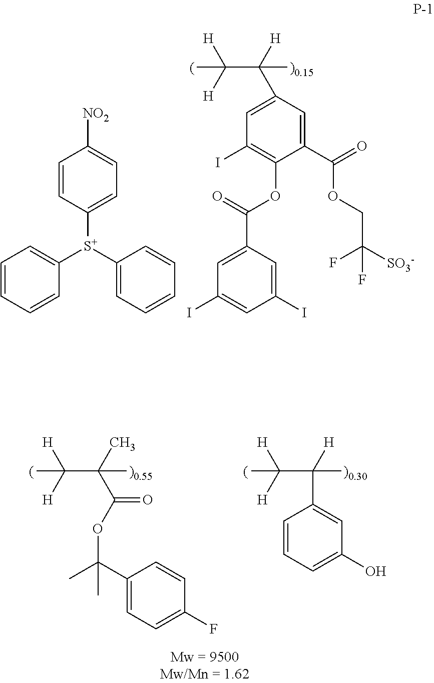

### Examples 2-2 to 2-26, Comparative Examples 2-1 to 2-18

### Synthesis of Polymers P-2 to P-26 and CP-1 to CP-18

Polymers shown in Tables 1 and 2 were produced in the same manner as in Example 2-1 except that the type and blending ratio of each monomer were changed.

TABLE 1

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| P-1 | PAG-1 | 15 | a1-1 | 55 | b1-1 | 30 | — | — | — | — | 9,500 | 1.62 |
| P-2 | PAG-2 | 15 | a1-1 | 55 | b1-1 | 30 | — | — | — | — | 9,400 | 1.62 |
| P-3 | PAG-3 | 15 | a1-1 | 55 | b1-1 | 30 | — | — | — | — | 9,700 | 1.63 |
| P-4 | PAG-4 | 15 | a1-1 | 55 | b1-1 | 30 | — | — | — | — | 9,300 | 1.63 |
| P-5 | PAG-5 | 15 | a1-1 | 55 | b1-1 | 30 | — | — | — | — | 9,300 | 1.61 |
| P-6 | PAG-6 | 15 | a1-1 | 55 | b1-1 | 30 | — | — | — | — | 9,400 | 1.62 |
| P-7 | PAG-7 | 15 | a1-1 | 55 | b1-1 | 30 | — | — | — | — | 9,300 | 1.61 |
| P-8 | PAG-8 | 15 | a1-1 | 55 | b1-1 | 30 | — | — | — | — | 9,200 | 1.61 |
| P-9 | PAG-9 | 15 | a1-1 | 55 | b1-1 | 30 | — | — | — | — | 9,400 | 1.62 |
| P-10 | PAG-1 | 15 | a1-2 | 55 | b1-1 | 30 | — | — | — | — | 9,200 | 1.60 |
| P-11 | PAG-1 | 15 | a1-3 | 55 | b1-1 | 30 | — | — | — | — | 9,000 | 1.62 |
| P-12 | PAG-1 | 15 | a2-1 | 55 | b1-1 | 30 | — | — | — | — | 9,500 | 1.62 |
| P-13 | PAG-1 | 15 | a3-1 | 45 | b1-1 | 40 | — | — | — | — | 8,800 | 1.64 |
| P-14 | PAG-2 | 15 | a1-1 | 55 | b1-2 | 30 | — | — | — | — | 9,100 | 1.61 |
| P-15 | PAG-2 | 15 | a1-1 | 55 | b1-3 | 30 | — | — | — | — | 9,100 | 1.62 |
| P-16 | PAG-2 | 15 | a1-1 | 55 | b1-4 | 30 | — | — | — | — | 9,200 | 1.63 |
| P-17 | PAG-1 | 15 | a1-1 | 30 | a2-1 | 20 | b1-1 | 35 | — | — | 9,300 | 1.62 |
| P-18 | PAG-3 | 15 | a1-1 | 35 | a3-1 | 15 | b1-2 | 35 | — | — | 9,500 | 1.62 |
| P-19 | PAG-4 | 15 | a1-2 | 30 | a2-1 | 15 | b1-3 | 40 | — | — | 9,200 | 1.62 |
| P-20 | PAG-6 | 10 | a1-1 | 35 | a2-1 | 15 | b1-1 | 30 | b2-1 | 10 | 9,100 | 1.62 |
| P-21 | PAG-7 | 15 | a1-2 | 35 | a3-1 | 15 | b1-2 | 25 | b2-2 | 10 | 9,200 | 1.62 |
| P-22 | PAG-9 | 15 | a1-1 | 50 | b1-1 | 30 | b2-3 | 5 | — | — | 8,900 | 1.63 |
| P-23 | PAG-1 | 5 | a1-1 | 55 | b1-2 | 40 | — | — | — | — | 9,500 | 1.62 |
| P-24 | PAG-2 | 5 | a1-1 | 30 | a1-3 | 25 | b1-2 | 40 | — | — | 9,300 | 1.61 |
| P-25 | PAG-3 | 5 | a1-2 | 30 | a2-1 | 20 | b1-4 | 35 | b2-1 | 10 | 9,200 | 1.61 |
| P-26 | PAG-7 | 5 | a1-1 | 35 | a3-1 | 15 | b1-1 | 30 | b2-2 | 15 | 9,200 | 1.62 |

TABLE 2

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| CP-1 | PAG-A | 15 | a1-1 | 55 | b1-1 | 30 | — | — | — | — | 9,500 | 1.62 |
| CP-2 | PAG-B | 15 | a1-1 | 55 | b1-1 | 30 | — | — | — | — | 9,100 | 1.61 |
| CP-3 | PAG-C | 15 | a1-1 | 55 | b1-1 | 30 | — | — | — | — | 9,300 | 1.63 |
| CP-4 | PAG-D | 15 | a1-1 | 55 | b1-1 | 30 | — | — | — | — | 9,100 | 1.62 |
| CP-5 | PAG-E | 15 | a1-1 | 55 | b1-1 | 30 | — | — | — | — | 9,000 | 1.65 |
| CP-6 | PAG-F | 15 | a1-1 | 55 | b1-1 | 30 | — | — | — | — | 9,500 | 1.62 |
| CP-7 | PAG-B | 15 | a1-2 | 55 | b1-1 | 30 | — | — | — | — | 9,600 | 1.61 |
| CP-8 | PAG-C | 15 | a3-1 | 45 | b1-1 | 40 | — | — | — | — | 9,700 | 1.63 |
| CP-9 | PAG-D | 15 | a1-1 | 55 | b1-3 | 30 | — | — | — | — | 9,500 | 1.63 |
| CP-10 | PAG-E | 15 | al- | 55 | b1-4 | 30 | — | — | — | — | 9,400 | 1.62 |
| CP-11 | PAG-B | 15 | a1-1 | 35 | a3-1 | 15 | b1-2 | 35 | — | — | 9,700 | 1.64 |
| CP-12 | PAG-D | 10 | a1-1 | 35 | a2-1 | 15 | b1-1 | 30 | b2-1 | 10 | 9,500 | 1.62 |
| CP-13 | PAG-C | 15 | a1-2 | 35 | a3-1 | 15 | b1-2 | 25 | b2-2 | 10 | 9,300 | 1.61 |
| CP-14 | PAG-F | 15 | a1-1 | 50 | b1-1 | 30 | b2-3 | 5 | — | — | 9,100 | 1.60 |
| CP-15 | PAG-A | 5 | a1-1 | 55 | b1-2 | 40 | — | — | — | — | 9,300 | 1.62 |
| CP-16 | PAG-D | 5 | a1-2 | 30 | a2-1 | 20 | b1-4 | 35 | b2-1 | 10 | 9,400 | 1.63 |
| CP-17 | a1-1 | 60 | b1-1 | 40 | — | — | — | — | — | — | 5,700 | 1.55 |
| CP-18 | a1-1 | 50 | b1-2 | 30 | b2-1 | 20 | — | — | — | — | 6,100 | 1.54 |

### [3] Preparation of Chemically Amplified Resist Composition

Examples 3-1 to 3-26, Comparative Examples 3-1 to 3-18 Chemically amplified resist compositions (R-1 to R-26, CR-1 to CR-18) in solution form were prepared by dissolving the base polymer (P-1 to P-26) containing the sulfonium salt monomer of the invention (PAG-1 to PAG-9), base polymer (CP-1 to CP-18) containing the comparative sulfonium salt monomer (PAG-A to PAG-F), photoacid generator (PAG-X and PAG-Y), and quencher (Q-1 to Q-4) in a solvent containing 0.01 wt % of surfactant A (Omnova Solutions, Inc.) in accordance with the formulation shown in Tables 3 and 4, and filtering through a Teflon® filter with a pore size of 0.2 μm.

TABLE 3

| | | | | | Photoacid | | | |
|---|---|---|---|---|---|---|---|---|
| | | Resist | Base polymer | Quencher | generator | Solvent 1 | Solvent 2 | Solvent 3 |
| | | composition | (pbw) | (pbw) | (pbw) | (pbw) | (pbw) | (pbw) |
| Example | 3-1 | R-1 | P-1 (80) | Q-1 (8.0) | — | PGMEA (2250) | EL (2800) | DAA (550) |
| | 3-2 | R-2 | P-2 (80) | Q-1 (8.2) | — | PGMEA (2250) | EL (2800) | DAA (550) |
| | 3-3 | R-3 | P-3 (80) | Q-1 (8.0) | — | PGMEA (2250) | EL (2800) | DAA (550) |
| | 3-4 | R-4 | P-4 (80) | Q-1 (7.8) | — | PGMEA (2250) | EL (2800) | DAA (550) |
| | 3-5 | R-5 | P-5 (80) | Q-1 (8.0) | — | PGMEA (2250) | EL (2800) | DAA (550) |
| | 3-6 | R-6 | P-6 (80) | Q-1 (8.0) | — | PGMEA (2250) | EL (2800) | DAA (550) |
| | 3-7 | R-7 | P-7 (80) | Q-1 (8.2) | — | PGMEA (2250) | EL (2800) | DAA (550) |
| | 3-8 | R-8 | P-8 (80) | Q-1 (8.0) | — | PGMEA (2250) | EL (2800) | DAA (550) |
| | 3-9 | R-9 | P-9 (80) | Q-1 (7.6) | — | PGMEA (2250) | EL (2800) | DAA (550) |
| | 3-10 | R-10 | P-10 (80) | Q-2 (8.2) | — | PGMEA (2250) | EL (2800) | DAA (550) |
| | 3-11 | R-11 | P-11 (80) | Q-3 (8.0) | — | PGMEA (2250) | EL (2800) | DAA (550) |
| | 3-12 | R-12 | P-12 (80) | Q-1 (8.0) | — | PGMEA (2250) | EL (2800) | DAA (550) |
| | 3-13 | R-13 | P-13 (80) | Q-1 (8.2) | — | PGMEA (2250) | EL (2800) | DAA (550) |
| | 3-14 | R-14 | P-14 (80) | Q-1 (8.0) | — | PGMEA (2250) | EL (2800) | DAA (550) |
| | 3-15 | R-15 | P-15 (80) | Q-3 (7.8) | — | PGMEA (2250) | EL (2800) | DAA (550) |
| | 3-16 | R-16 | P-16 (80) | Q-2 (8.0) | — | PGMEA (2250) | EL (2800) | DAA (550) |
| | 3-17 | R-17 | P-17 (80) | Q-1 (8.0) | — | PGMEA (2250) | EL (2800) | DAA (550) |
| | 3-18 | R-18 | P-18 (80) | Q-1 (7.8) | — | PGMEA (2250) | EL (2800) | DAA (550) |
| | 3-19 | R-19 | P-19 (80) | Q-2 (8.0) | — | PGMEA (2250) | EL (2800) | DAA (550) |
| | 3-20 | R-20 | P-20 (80) | Q-3 (7.8) | PAG-Y (15) | PGMEA (2250) | EL (2800) | DAA (550) |
| | 3-21 | R-21 | P-21 (80) | Q-1 (4.0) | — | PGMEA (2250) | EL (2800) | DAA (550) |
| | | | | Q-4 (3.8) | | | | |
| | 3-22 | R-22 | P-22 (80) | Q-1 (8.2) | — | PGMEA (2250) | EL (2800) | DAA (550) |
| | 3-23 | R-23 | P-23 (80) | Q-3 (7.6) | — | PGMEA (2250) | EL (2800) | DAA (550) |
| | 3-24 | R-24 | P-24 (80) | Q-1 (8.0) | PAG-X (10) | PGMEA (2250) | EL (2800) | DAA (550) |
| | 3-25 | R-25 | P-25 (80) | Q-2 (8.2) | PAG-X (10) | PGMEA (2250) | EL (2800) | DAA (550) |
| | 3-26 | R-26 | P-26 (80) | Q-3 (8.0) | PAG-Y (15) | PGMEA (2250) | EL (2800) | DAA (550) |

TABLE 4

| | | | | | Photoacid | | | |
|---|---|---|---|---|---|---|---|---|
| | | Resist | Base polymer | Quencher | generator | Solvent 1 | Solvent 2 | Solvent 3 |
| | | composition | (pbw) | (pbw) | (pbw) | (pbw) | (pbw) | (pbw) |
| Comparative | 3-1 | CR-1 | CP-1 (80) | Q-1 (8.0) | — | PGMEA (2250) | EL (2800) | DAA (550) |
| Example | 3-2 | CR-2 | CP-2 (80) | Q-1 (8.2) | — | PGMEA (2250) | EL (2800) | DAA (550) |
| | 3-3 | CR-3 | CP-3 (80) | Q-1 (8.0) | — | PGMEA (2250) | EL (2800) | DAA (550) |
| | 3-4 | CR-4 | CP-4 (80) | Q-1 (7.8) | — | PGMEA (2250) | EL (2800) | DAA (550) |
| | 3-5 | CR-5 | CP-5 (80) | Q-1 (8.0) | — | PGMEA (2250) | EL (2800) | DAA (550) |
| | 3-6 | CR-6 | CP-6 (80) | Q-1 (8.2) | — | PGMEA (2250) | EL (2800) | DAA (550) |
| | 3-7 | CR-7 | CP-7 (80) | Q-2 (8.2) | — | PGMEA (2250) | EL (2800) | DAA (550) |
| | 3-8 | CR-8 | CP-8 (80) | Q-1 (8.2) | — | PGMEA (2250) | EL (2800) | DAA (550) |
| | 3-9 | CR-9 | CP-9 (80) | Q-3 (7.8) | — | PGMEA (2250) | EL (2800) | DAA (550) |
| | 3-10 | CR-10 | CP-10 (80) | Q-2 (8.0) | — | PGMEA (2250) | EL (2800) | DAA (550) |
| | 3-11 | CR-11 | CP-11 (80) | Q-1 (8.0) | — | PGMEA (2250) | EL (2800) | DAA (550) |
| | 3-12 | CR-12 | CP-12 (80) | Q-3 (7.8) | PAG-Y (15) | PGMEA (2250) | EL (2800) | DAA (550) |
| | 3-13 | CR-13 | CP-13 (80) | Q-1 (4.0) | — | PGMEA (2250) | EL (2800) | DAA (550) |
| | | | | Q-4 (3.8) | | | | |
| | 3-14 | CR-14 | CP-14 (80) | Q-1 (8.2) | — | PGMEA (2250) | EL (2800) | DAA (550) |
| | 3-15 | CR-15 | CP-15 (80) | Q-3 (7.6) | — | PGMEA (2250) | EL (2800) | DAA (550) |
| | 3-16 | CR-16 | CP-16 (80) | Q-2 (8.2) | PAG-X (10) | PGMEA (2250) | EL (2800) | DAA (550) |
| | 3-17 | CR-17 | CP-17 (80) | Q-1 (8.0) | PAG-X (24) | PGMEA (2250) | EL (2800) | DAA (550) |
| | 3-18 | CR-18 | CP-18 (80) | Q-1 (8.0) | PAG-Y (24) | PGMEA (2250) | EL (2800) | DAA (550) |

The solvents, photoacid generators PAG-X and PAG-Y, quenchers Q-1 to Q-4, and surfactant A in Tables 3 and 4 are identified below.

### Solvents:

PGMEA (propylene glycol monomethyl ether acetate)
EL (ethyl lactate)
DAA (diacetone alcohol)

### Photoacid Generators: PAG-X and PAG-Y

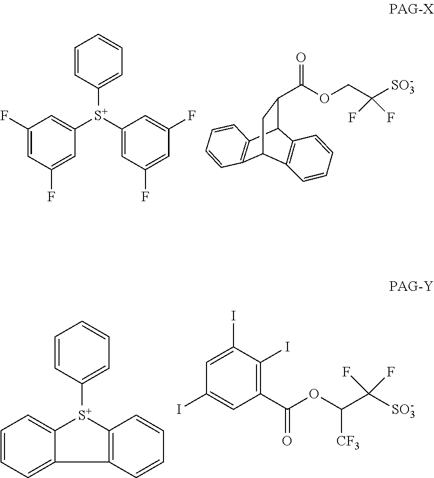

### Quenchers: Q-1 to Q-4

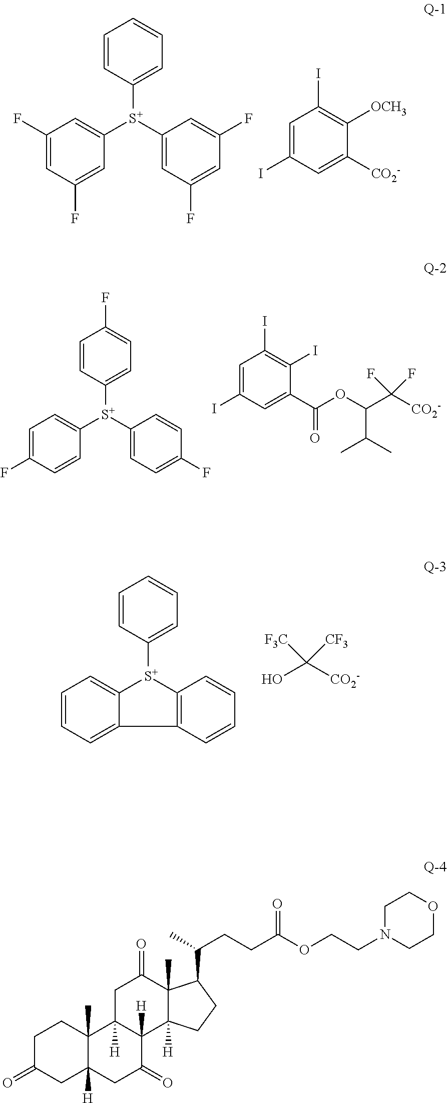

### Surfactants A:

3-methyl-3-(2,2,2-trifluoroethoxymethyl)-oxetane/tetrahydrofuran/
2,2-dimethyl-1,3-propane diol copolymer (manufactured by Omnova Solutions, Inc.)

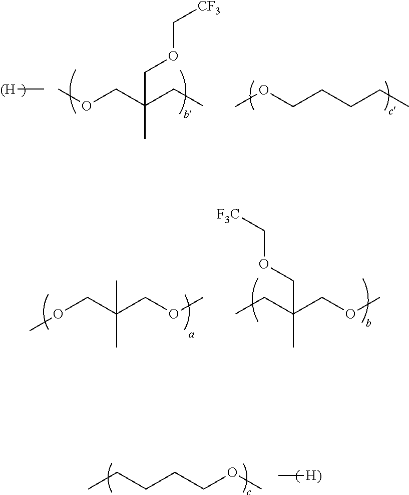

a: (b+b′): (c+c′)=1:4-7:0.01-1 (molar ratio)
Mw=1,500

### [4] EUV Lithography Test (1)

### Examples 4-1 to 4-26, Comparative Examples 4-1 to 4-18

Each of the chemically amplified resist compositions (R-1 to R-26, CR-1 to CR-18) shown in Tables 3 and 4 was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3400 (ASML, NA 0.33, σ 0.9/0.6, dipole illumination), the resist film was exposed to EUV through a mask bearing an LS pattern having a size of 18 nm and a pitch of 36 nm (on-wafer size) while varying the dose and focus (dose pitch: 1 mJ/cm², focus pitch: 0.020 μm). The resist film was baked (PEB) at the temperature shown in Tables 5 and 6 for 60 seconds and puddle developed in a 2.38 wt % TMAH aqueous solution for 30 seconds, rinsed with a rinse fluid containing surfactant, and spin dried to form a positive pattern.

The LS pattern as developed was observed under CD-SEM (CG6300, Hitachi High-Technologies Corp.) whereupon sensitivity, EL, LWR, depth of focus (DOF) and collapse limit were evaluated by the following methods. In addition, development defects of the obtained LS pattern were evaluated. The results are shown in Tables 5 and 6.

### [Evaluation of Sensitivity]

The optimum dose Eop (mJ/cm²) which provided an LS pattern with a line width of 18 nm and a pitch of 36 nm was determined as an index of sensitivity. A smaller value indicates a higher sensitivity.

### [Evaluation of EL]

The exposure dose which provided an LS pattern with a space width of 18 nm+10% (16.2 to 19.8 nm) was determined. EL (%) is calculated from the exposure doses according to the following equation:

$$EL ( \% ) = ( \vert {E}_{1} - {E}_{2} \vert / Eop ) \times 100$$

wherein E₁ is an optimum exposure dose which provides an LS pattern with a line width of 16.2 nm and a pitch of 36 nm, E₂ is an optimum exposure dose which provides an LS pattern with a line width of 19.8 nm and a pitch of 36 nm, and Eop is an optimum exposure dose which provides an LS pattern with a line width of 18 nm and a pitch of 36 nm. A greater value indicates better performance.

### [Evaluation of LWR]

For the LS pattern formed by exposure at the optimum dose Eop, the line width was measured at 10 longitudinally spaced apart points, from which a 3-fold value (3σ) of the standard deviation (o) was determined and reported as LWR. A smaller value of 3σ indicates a pattern having small roughness and uniform line width.

### [Evaluation of DOF]

As an index of depth of focus, a range of focus which provided an LS pattern with a size of 18 nm+10% (i.e., 16.2 to 19.8 nm) was determined. A greater value indicates a wider depth of focus.

### [Evaluation of Line Pattern Collapse Limit]

For the LS pattern formed by exposure at the dose corresponding to the optimum focus, the line width was measured at 10 longitudinally spaced apart points. The minimum line width above which lines could be resolved without collapse was determined and reported as collapse limit. A smaller value indicates better collapse limit.

### [Evaluation of Development Defect]

Using a defect inspection apparatus KLA2360 (trade name) manufactured by KLA-Tencor Corporation, the LS pattern with a line width of 18 nm and a pitch of 36 nm formed with the optimum dose, the pixel size in the defect inspection apparatus was set at 0.16 μm and the threshold value at 20. Defects extracted from differences generated by superimposition between a comparative image and the pixel unit were detected, and the number of defects per unit area (defects/cm²) was calculated. Thereafter, development defects were classified and extracted from all defects by defect review, and the number of development defects per unit area (defects/cm²) was calculated. The evaluation marks A, B, C and D were given when the value was less than 0.5, 0.5 or more and less than 1.0, 1.0 or more and less than 5.0, and 5.0 or more, respectively. The smaller the value, the more favorable the performance exhibited.

TABLE 5

| | | | | Optimum | | | | | |
|---|---|---|---|---|---|---|---|---|---|
| | | | PEB | exposure | | | | Collapse | |
| | | Resist | temp. | dose | EL | LWR | DOF | limit | Development |
| | | composition | (° C.) | (mJ/cm²) | (%) | (nm) | (nm) | (nm) | defects |
| Example | 4-1 | R-1 | 95 | 32 | 18 | 2.2 | 120 | 10.9 | A |
| | 4-2 | R-2 | 100 | 32 | 19 | 2.3 | 110 | 10.7 | A |
| | 4-3 | R-3 | 100 | 32 | 17 | 2.3 | 120 | 10.8 | A |
| | 4-4 | R-4 | 95 | 32 | 18 | 2.4 | 110 | 11.2 | A |
| | 4-5 | R-5 | 105 | 34 | 17 | 2.4 | 100 | 11.1 | A |
| | 4-6 | R-6 | 100 | 33 | 17 | 2.4 | 120 | 11.3 | A |
| | 4-7 | R-7 | 95 | 32 | 18 | 2.3 | 110 | 11.3 | A |
| | 4-8 | R-8 | 95 | 33 | 19 | 2.4 | 100 | 11.0 | A |
| | 4-9 | R-9 | 100 | 33 | 17 | 2.3 | 110 | 11.1 | A |
| | 4-10 | R-10 | 100 | 33 | 18 | 2.3 | 120 | 11.2 | A |
| | 4-11 | R-11 | 100 | 33 | 19 | 2.2 | 120 | 11.2 | A |
| | 4-12 | R-12 | 95 | 32 | 17 | 2.3 | 110 | 10.7 | A |
| | 4-13 | R-13 | 105 | 33 | 18 | 2.2 | 120 | 10.6 | A |
| | 4-14 | R-14 | 100 | 32 | 17 | 2.3 | 100 | 10.8 | A |
| | 4-15 | R-15 | 95 | 34 | 17 | 2.3 | 110 | 10.9 | A |
| | 4-16 | R-16 | 95 | 32 | 18 | 2.4 | 120 | 11.3 | A |
| | 4-17 | R-17 | 100 | 33 | 17 | 2.2 | 110 | 11.1 | A |
| | 4-18 | R-18 | 95 | 34 | 18 | 2.4 | 110 | 11.4 | A |
| | 4-19 | R-19 | 95 | 33 | 18 | 2.4 | 120 | 11.1 | A |
| | 4-20 | R-20 | 100 | 33 | 17 | 2.3 | 110 | 10.8 | A |
| | 4-21 | R-21 | 100 | 34 | 19 | 2.2 | 110 | 10.9 | A |
| | 4-22 | R-22 | 100 | 33 | 17 | 2.3 | 110 | 11.4 | A |
| | 4-23 | R-23 | 95 | 32 | 18 | 2.4 | 110 | 11.2 | A |
| | 4-24 | R-24 | 95 | 33 | 18 | 2.5 | 120 | 11.2 | A |
| | 4-25 | R-25 | 100 | 33 | 17 | 2.4 | 110 | 11.1 | A |
| | 4-26 | R-26 | 100 | 32 | 19 | 2.3 | 110 | 10.9 | A |

TABLE 6

| | | | | Optimum | | | | | |
|---|---|---|---|---|---|---|---|---|---|
| | | | PEB | exposure | | | | Collapse | |
| | | Resist | temp. | dose | EL | LWR | DOF | limit | Development |
| | | composition | (° C.) | (mJ/cm²) | (%) | (nm) | (nm) | (nm) | defects |
| Comparative | 4-1 | CR-1 | 95 | 40 | 13 | 3.1 | 90 | 12.7 | B |
| Example | 4-2 | CR-2 | 100 | 38 | 14 | 2.8 | 80 | 12.2 | C |
| | 4-3 | CR-3 | 100 | 37 | 14 | 2.7 | 80 | 13.3 | C |
| | 4-4 | CR-4 | 100 | 36 | 15 | 2.8 | 90 | 13.1 | B |
| | 4-5 | CR-5 | 95 | 37 | 13 | 2.8 | 80 | 12.4 | C |
| | 4-6 | CR-6 | 100 | 37 | 11 | 3.5 | 60 | 12.2 | B |
| | 4-7 | CR-7 | 100 | 36 | 14 | 2.7 | 90 | 12.3 | C |
| | 4-8 | CR-8 | 100 | 37 | 13 | 2.6 | 80 | 12.5 | C |
| | 4-9 | CR-9 | 95 | 38 | 15 | 2.7 | 90 | 13.6 | B |
| | 4-10 | CR-10 | 100 | 37 | 14 | 2.8 | 80 | 12.2 | B |
| | 4-11 | CR-11 | 100 | 37 | 14 | 2.7 | 80 | 12.3 | B |
| | 4-12 | CR-12 | 100 | 36 | 14 | 2.8 | 80 | 13.2 | B |
| | 4-13 | CR-13 | 95 | 36 | 13 | 2.9 | 90 | 12.3 | B |
| | 4-14 | CR-14 | 105 | 37 | 11 | 3.5 | 50 | 12.8 | C |
| | 4-15 | CR-15 | 100 | 40 | 13 | 3.1 | 70 | 12.4 | C |
| | 4-16 | CR-16 | 95 | 39 | 15 | 2.8 | 80 | 13.3 | B |
| | 4-17 | CR-17 | 95 | 41 | 10 | 3.4 | 60 | 12.4 | B |
| | 4-18 | CR-18 | 100 | 40 | 10 | 3.5 | 60 | 12.2 | B |

It is demonstrated in Tables 5 and 6 that chemically amplified resist compositions comprising the polymer composed of the sulfonium salt monomer of the invention exhibit a high sensitivity and improved values of EL, LWR and DOF. Small values of collapse limit attest that in forming a small-size pattern, the pattern is resistant to collapse. Furthermore, it is demonstrated that development defects were also suppressed. The chemically amplified resist compositions of the invention are suitable as materials for EUV lithography.

### [5] EUV Lithography Test (2)

### Examples 5-1 to 5-26, Comparative Examples 5-1 to 5-18

Each of the chemically amplified resist compositions (R-1 to R-26, CR-1 to CR-18) shown in Tables 3 and 4 was spin coated on a silicon substrate having a 20-nm coating of silicon-containing spin-on hard mask SHB-A940 (Shin-Etsu Chemical Co., Ltd., silicon content 43 wt %) and prebaked on a hotplate at 105° C. for 60 seconds to form a resist film of 50 nm thick. Using an EUV scanner NXE3400 (ASML, NA 0.33, @ 0.9/0.6, quadrupole illumination), the resist film was exposed to EUV through a mask bearing a hole pattern having a pitch of 46 nm+20% bias (on-wafer size). The resist film was baked (PEB) on a hotplate at the temperature shown in Tables 7 and 8 for 60 seconds and developed in a 2.38 wt % TMAH aqueous solution for 30 seconds to form a hole pattern having a size of 23 nm.

The pattern as developed was observed under CD-SEM (CG6300, Hitachi High-Technologies Corp.). The dose (mJ/cm²) at which a pattern with a hole size of 23 nm was printed was determined as an index of sensitivity. The size of 50 holes was measured, from which a 3-fold value (3σ) of the standard deviation (σ) was determined as CDU. The results are shown in Tables 7 and 8.

TABLE 7

| | | | PEB | Optimum | |
|---|---|---|---|---|---|
| | | Resist | temp. | exposure dose | CDU |
| | | composition | (° C.) | (mJ/cm²) | (nm) |
| Example | 5-1 | R-1 | 95 | 22 | 2.2 |
| | 5-2 | R-2 | 95 | 22 | 2.3 |
| | 5-3 | R-3 | 90 | 23 | 2.4 |
| | 5-4 | R-4 | 90 | 22 | 2.3 |
| | 5-5 | R-5 | 90 | 23 | 2.4 |
| | 5-6 | R-6 | 95 | 24 | 2.3 |
| | 5-7 | R-7 | 95 | 23 | 2.4 |
| | 5-8 | R-8 | 90 | 25 | 2.3 |
| | 5-9 | R-9 | 95 | 22 | 2.4 |
| | 5-10 | R-10 | 95 | 23 | 2.3 |
| | 5-11 | R-11 | 95 | 24 | 2.4 |
| | 5-12 | R-12 | 90 | 23 | 2.4 |
| | 5-13 | R-13 | 90 | 22 | 2.3 |
| | 5-14 | R-14 | 90 | 23 | 2.3 |
| | 5-15 | R-15 | 90 | 23 | 2.3 |
| | 5-16 | R-16 | 85 | 22 | 2.4 |
| | 5-17 | R-17 | 95 | 24 | 2.2 |
| | 5-18 | R-18 | 95 | 23 | 2.2 |
| | 5-19 | R-19 | 90 | 22 | 2.3 |
| | 5-20 | R-20 | 95 | 23 | 2.5 |
| | 5-21 | R-21 | 95 | 24 | 2.3 |
| | 5-22 | R-22 | 95 | 23 | 2.4 |
| | 5-23 | R-23 | 95 | 22 | 2.3 |
| | 5-24 | R-24 | 90 | 23 | 2.3 |
| | 5-25 | R-25 | 95 | 22 | 2.4 |
| | 5-26 | R-26 | 95 | 23 | 2.2 |

TABLE 8

| | | | PEB | Optimum | |
|---|---|---|---|---|---|
| | | Resist | temp. | exposure dose | CDU |
| | | composition | (° C.) | (mJ/cm²) | (nm) |
| Comparative | 5-1 | CR-1 | 95 | 31 | 3.1 |
| Example | 5-2 | CR-2 | 95 | 28 | 2.8 |
| | 5-3 | CR-3 | 95 | 27 | 2.7 |
| | 5-4 | CR-4 | 90 | 27 | 2.8 |
| | 5-5 | CR-5 | 90 | 28 | 2.8 |
| | 5-6 | CR-6 | 95 | 25 | 3.4 |
| | 5-7 | CR-7 | 90 | 28 | 2.8 |
| | 5-8 | CR-8 | 90 | 27 | 2.7 |
| | 5-9 | CR-9 | 90 | 26 | 2.8 |
| | 5-10 | CR-10 | 95 | 28 | 2.8 |
| | 5-11 | CR-11 | 95 | 28 | 2.7 |
| | 5-12 | CR-12 | 95 | 29 | 2.9 |
| | 5-13 | CR-13 | 85 | 27 | 2.8 |
| | 5-14 | CR-14 | 95 | 25 | 3.4 |
| | 5-15 | CR-15 | 95 | 31 | 3.2 |
| | 5-16 | CR-16 | 90 | 29 | 2.9 |
| | 5-17 | CR-17 | 95 | 32 | 3.3 |
| | 5-18 | CR-18 | 95 | 32 | 3.2 |

It is demonstrated in Tables 7 and 8 that chemically amplified resist compositions comprising the polymer composed of the sulfonium salt monomer of the invention exhibit a high sensitivity and satisfactory CDU.

### [6] Evaluation of Dry Etching Resistance

### Examples 6-1 to 6-26, Comparative Examples 6-1 to 6-18

Each of polymer solutions obtained by dissolving 2 g of each of the polymers (polymers P-1 to P-26, comparative polymers CP-1 to CP-18) shown in Tables 1 and 2 in g of cyclohexanone and filtering the solution through a 0.2 μm-sized filter was deposited on a silicon substrate by spin coating to form a film having a thickness of 300 nm. Dry etching resistance was evaluated under the following conditions.

Etching Test with CHF₃/CF₄ Based Gas:

A difference in a thickness of the polymer film before and after etching was determined using a dry etching apparatus TE-8500P manufactured by Tokyo Electron, Ltd.

Etching conditions are as follows.

| | | |
|---|---|---|
| Chamber pressure | 40 | Pa |
| RF Power | 1000 | W |
| Gap | 9 | mm |
| CHF₃ Gas flow rate | 30 | mL/min |
| CF₄ Gas flow rate | 30 | mL/min |
| Ar Gas flow rate | 100 | mL/min |
| Time | 60 | sec |

In this evaluation, a film having a small film thickness difference, that is, a film having a small reduction amount indicates that the etching resistance is high.

The results for dry etching resistance are shown in Tables 9 and 10.

TABLE 9

| | | | CHF₃/CF₄-based gas |
|---|---|---|---|
| | | Polymer | etching rate (nm/min) |
| Example | 6-1 | P-1 | 96 |
| | 6-2 | P-2 | 97 |
| | 6-3 | P-3 | 96 |
| | 6-4 | P-4 | 95 |
| | 6-5 | P-5 | 96 |
| | 6-6 | P-6 | 96 |
| | 6-7 | P-7 | 97 |
| | 6-8 | P-8 | 95 |
| | 6-9 | P-9 | 96 |
| | 6-10 | P-10 | 97 |
| | 6-11 | P-11 | 96 |
| | 6-12 | P-12 | 97 |
| | 6-13 | P-13 | 96 |
| | 6-14 | P-14 | 98 |
| | 6-15 | P-15 | 97 |
| | 6-16 | P-16 | 95 |
| | 6-17 | P-17 | 98 |
| | 6-18 | P-18 | 97 |
| | 6-19 | P-19 | 96 |
| | 6-20 | P-20 | 98 |
| | 6-21 | P-21 | 96 |
| | 6-22 | P-22 | 97 |
| | 6-23 | P-23 | 96 |
| | 6-24 | P-24 | 98 |
| | 6-25 | P-25 | 95 |
| | 6-26 | P-26 | 97 |

TABLE 10

| | | | CHF₃/CF₄-based gas |
|---|---|---|---|
| | | Polymer | etching rate (nm/min) |
| Comparative | 6-1 | CP-1 | 114 |
| Example | 6-2 | CP-2 | 98 |
| | 6-3 | CP-3 | 97 |
| | 6-4 | CP-4 | 98 |
| | 6-5 | CP-5 | 109 |
| | 6-6 | CP-6 | 110 |
| | 6-7 | CP-7 | 98 |
| | 6-8 | CP-8 | 97 |
| | 6-9 | CP-9 | 99 |
| | 6-10 | CP-10 | 105 |
| | 6-11 | CP-11 | 98 |
| | 6-12 | CP-12 | 99 |
| | 6-13 | CP-13 | 97 |
| | 6-14 | CP-14 | 110 |
| | 6-15 | CP-15 | 108 |
| | 6-16 | CP-16 | 99 |
| | 6-17 | CP-17 | 109 |
| | 6-18 | CP-18 | 108 |

It is demonstrated in Tables 9 and 10 that the polymers of the invention have excellent dry etching resistance to a CHF₃/CF₄ gas.

Japanese Patent Application No. 2024-131999 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. A sulfonium salt monomer having the formula (A): 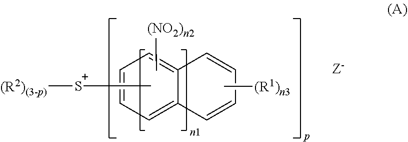
wherein p is 1, 2, or 3, n1 is 0 or 1, n2 is 1 or 2, n3 is 0, 1, 2, or 3, meeting 1≤n2+n3≤5 in case of n1=0 and 1≤n2+n3≤7 in case of n1=1,
R¹ is halogen, cyano, hydroxy, carboxy, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom, and when n3 is 2 or 3, a plurality of R¹ may be identical or different and two R¹ may bond together to form a ring with the carbon atoms to which they are attached,
R² is halogen or a C₁-C₃₀ hydrocarbyl group which may contain a heteroatom, when p is 1, two R² may be identical or different, and any two of three substituents bonded to S+ may bond together to form a ring with the sulfur atoms to which they are attached, and
Z⁻ is a fluoroalkanesulfonate anion having an aromatic vinyl structure and an iodine atom.

2. The sulfonium salt monomer of claim 1 having the formula (A1): 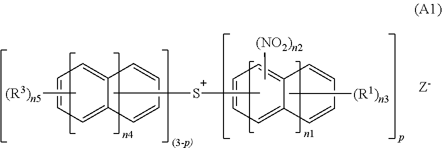
wherein p, n1 to n3, R¹, and Z⁻ are as defined above,
n4 is 0 or 1, n5 is 0, 1, 2, 3, 4, or 5, and
R³ is halogen, cyano, hydroxy, carboxy, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom, and when n5 is 2, 3, 4, or 5, a plurality of R³ may be identical or different and two R³ may bond together to form a ring with the carbon atoms to which they are attached.

3. The sulfonium salt monomer of claim 1 wherein Z is an anion having the formula (Z): 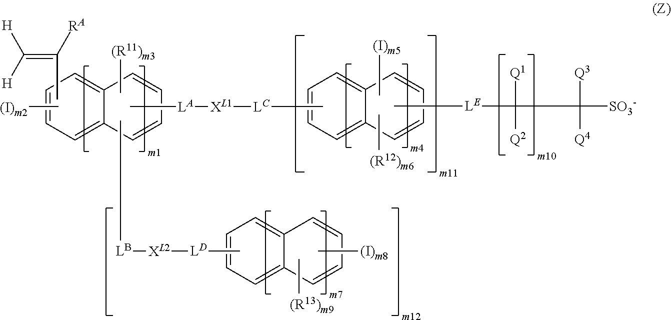
wherein m1 is 0 or 1, m2 is 0, 1, 2, 3, or 4, m3 is 0, 1, 2, or 3, m4 is 0 or 1, m5 is 0, 1, 2, 3, or 4, m6 is 0, 1, 2, or 3, m7 is 0 or 1, m8 is 1, 2, 3, or 4, m9 is 0, 1, 2, or 3, m10 is 0, 1, 2, 3, or 4, m11 is 0 or 1, m12 is 0 or 1, meeting 0≤m2+m3+m12≤4 in case of m1=0 and 0≤m2+m3+m12≤6 in case of m1=1, 0≤m5+m6≤4 in case of m4-0 and 0≤m5+m6≤6 in case of m4=1, 0≤m8+m9≤5 in case of m7-0 and 0≤m8+m9≤7 in case of m7=1, and also meeting 1≤m2+m5+m8≤4,
R^{A} is hydrogen, fluorine, methyl or trifluoromethyl,
R¹¹, R¹², and R¹³ are each independently halogen exclusive of iodine, nitro, cyano, hydroxy, carboxy, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom, when m3 is 2 or 3, a plurality of R¹¹ may be identical or different and two R¹¹ may bond together to form a ring with the carbon atoms to which they are attached, when m6 is 2 or 3, a plurality of R¹² may be identical or different and two R¹² may bond together to form a ring with the carbon atoms to which they are attached, and when m9 is 2 or 3, a plurality of R¹³ may be identical or different and two R¹³ may bond together to form a ring with the carbon atoms to which they are attached,
L^{A}, L^{B}, L^{C}, L^{D}, and L^{E} are each independently a single bond, ether bond, ester bond, sulfonate ester bond, amide bond, sulfonamide bond, carbonate bond or carbamate bond,
X^{L1} and X^{L2} are each independently a single bond or a C₁-C₄₀ hydrocarbylene group which may contain a heteroatom,
Q¹ and Q² are each independently hydrogen, fluorine, or a C₁-C₆ fluorinated saturated hydrocarbyl group, and
Q³ and Q⁴ are each independently fluorine or a C₁-C₆ fluorinated saturated hydrocarbyl group,
excluding that m11 and m12 are 0 at the same time, and excluding that L^{A}, L^{B}, L^{C}, L^{D} X^{L1} and X^{L2} are a single bond at the same time.

4. A monomer photoacid generator comprising the sulfonium salt monomer of claim 1.

5. A polymer comprising repeat units derived from the monomer photoacid generator of claim 4.

6. The polymer of claim 5, further comprising at least one selected from repeat units having the formula (a1), repeat units represented by the formula (a2), and repeat units represented by the formula (a3): 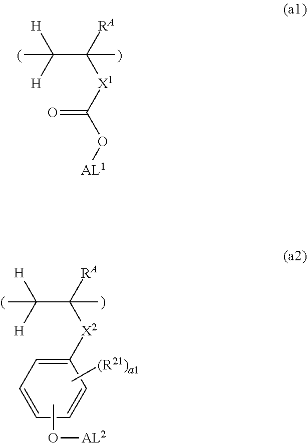
wherein R^{A} is each independently hydrogen, fluorine, methyl or trifluoromethyl,
X¹ is a single bond, phenylene group, naphthylene group, or *—C(═O)—O—X¹¹—, the phenylene or naphthylene group may be substituted by a hydroxy moiety, nitro moiety, cyano moiety, optionally fluorinated C₁-C₁₀ saturated hydrocarbyl moiety, optionally fluorinated C₁-C₁₀ saturated hydrocarbyloxy moiety, or halogen, X¹¹ is a C₁-C₁₀ saturated hydrocarbylene group which may contain a hydroxy moiety, ether bond, ester bond or lactone ring, or phenylene or naphthylene group,
X² is a single bond or *—C(═O)—O—,
the asterisk (*) designates a point of attachment to the carbon atom in the backbone,
R²¹ is halogen, cyano, hydroxy, nitro, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R²¹ may be identical or different when a1 is 2, 3, or 4,
AL¹ and AL² are each independently an acid labile group, and
a1 is 0, 1, 2, 3, or 4; 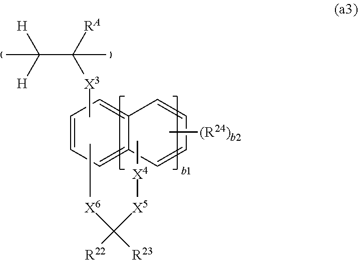
wherein b1 is 0 or 1, b2 is 0, 1, 2, or 3 in case of b1=0, and is 0, 1, 2, 3, 4, or 5 in case of b1=1,
R^{A} is hydrogen, fluorine, methyl or trifluoromethyl,
X³ is a single bond, *—C(═O)—O—, or *—C(═O)—NH—, * designates a point of attachment to the carbon atom in the backbone,
X⁴ is a single bond, a C₁-C₄ aliphatic hydrocarbylene group, carbonyl group, sulfonyl group, or a group obtained by combining the foregoing,
X⁵ and X⁶ are each independently oxygen or sulfur, X⁴ and X⁶ are bonded to vicinal carbon atoms on the aromatic ring,
R²² and R²³ are each independently hydrogen or a C₁-C₂₀ hydrocarbyl group which may contain a heteroatom, R²² and R²³ may bond together to form a ring with the carbon atoms to which they are attached, and
R²⁴ is halogen, hydroxy, cyano, nitro, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{24A}) (R^{24B}), R^{24A} and R^{24B} are each independently hydrogen or a C₁-C₆ hydrocarbyl group, and when b2 is 2 or more, a plurality of R²⁴ may be identical or different and a plurality of R²⁴ may bond together to form a ring with the carbon atoms on the aromatic ring to which they are attached.

7. The polymer of claim 5, further comprising at least one selected from repeat units having the formula (b1) and repeat units having the formula (b2): 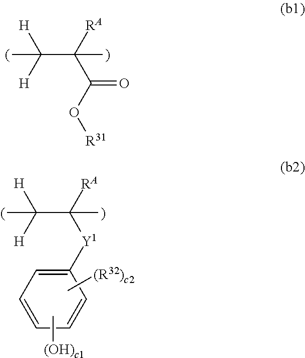
wherein R^{A} is each independently hydrogen, fluorine, methyl or trifluoromethyl,
Y¹ is a single bond or *—C(═O)—O—, the asterisk (*) designates a point of attachment to the carbon atom in the backbone,
R³¹ is hydrogen or a C₁-C₂₀ group containing at least one structure selected from among hydroxy other than phenolic hydroxy, cyano, carbonyl, carboxy, ether bond, ester bond, sulfonate ester bond, carbonate bond, lactone ring, sultone ring and carboxylic anhydride (—C(═O)—O—C(═O)—),
R³² is halogen, carboxy, nitro, cyano, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R³² may be identical or different when c2 is 2, 3, or 4, and
c1 is 1, 2, 3, or 4 and c2 is 0, 1, 2, 3, or 4, meeting 1≤c1+c2≤5.

8. A chemically amplified resist composition comprising (A) a base polymer comprising the polymer of claim 5.

9. The chemically amplified resist composition of claim 8, further comprising (B) an organic solvent.

10. The chemically amplified resist composition of claim 8, further comprising (C) a quencher.

11. The chemically amplified resist composition of claim 8, further comprising (D) a photoacid generator.

12. The chemically amplified resist composition of claim 8, further comprising (E) a surfactant.

13. A pattern forming process comprising the steps of applying the chemically amplified resist composition of claim 8 onto a substrate to form a resist film thereon, exposing the resist film to high-energy radiation, and developing the exposed resist film in a developer.

14. The pattern forming process of claim 13 wherein the high-energy radiation is KrF excimer laser, ArF excimer laser, electron beam, or extreme ultraviolet of wavelength 3 to 15 nm.
